(19) United States
(12) Patent Application Publication
(10) Pub. No.: US 2004/0088746 A1
(43) Pub. Date: May 6, 2004

(54) Apoptosis-inducing dna sequences

(76) Inventors: Stefan Grimm, Munchen (DE); Nicole Schonfeld, Munchen (DE); Erik Braziulis, Martinsried (DE); Ursula Cramer, Munchen (DE); Andreas Gewies, Munchen (DE); Frank Voss, Munchen (DE); Thomas Mund, Munchen (DE); Timur Albayrak, Munchen (DE); Hendrik Gille, Munchen (DE); Matthias Klein, Martinsried (DE); Manuel Bauer, Munchen (DE)

Correspondence Address: Arent Fox Kintner, Plotkin & Kahn, Suite 600, 1050 Connecticut Avenue NW, Washington, DC, 20036-5339, US

(21) Appl. No.: 10/332,859

(22) PCT Filed: Jul. 13, 2001
(86) PCT No.: PCT/EP01/08170

(30) Foreign Application Priority Data
Jul. 14, 2000 (DE) 10034303.1

(51) Int. Cl. A01K 67/027; A61K 48/00

(52) U.S. Cl. 800/18; 435/320.1; 514/44

(57) ABSTRACT

The invention relates to novel apoptosis-associated and in particular apoptosis-inducing nucleic acid sequences, to polypeptides encoded thereby and to the use thereof for providing diagnostic and therapeutic agents. The invention also relates to cell systems and transgenic animals and the use thereof for the genetic and/or pharmacological investigation of apoptosis-associated disorders.

### DESCRIPTION

The invention relates to novel apoptosis-associated and in particular apoptosis-inducing nucleic acid sequences, to polypeptides encoded thereby and to the use thereof for providing diagnostic and therapeutic agents. The invention also relates to transgenic cell systems and transgenic animals and the use thereof for the genetic and/or pharmacological investigation of apoptosis-associated disorders.

Apoptosis is the genetically encoded suicide program which is induced in eukaryotic cells under particular physiological or pathological conditions. The induction of apoptosis must be regulated with extraordinary precision, because a hyperactivity can lead to degenerative disorders. On the other hand, a reduced induction of apoptosis can contribute to tumor progression.

Various low molecular weight inducers of apoptosis have already been described. An important class are tumor cytostatics. However, in most cases it is not known how said cytostatics or other substances can induce apoptosis.

The identification of apoptosis-inducing genes or other dominant genes having a non-selectable activity is problematic, since stable recombinant expression of such genes in a target cell is possible only with great difficulty, if at all. It is therefore necessary to use specific screening methods for identifying such genes. For this purpose, various in-vitro methods have already been developed (King et al., Science 277 (1997), 973-974 and Lustig et al., Meth. Enzymol. 283 (1997), 83-99). Other research groups have generated transgenic mice which contain multiple transgenes whose functions are determined by studying the phenotype (Simonet et al., Cell 89 (1997), 309-319 and Smith et al., Nat. Genet. 16 (1997), 28-36). A disadvantage of the in-vitro methods is that the results obtained do not readily correlate with cell-biological effects regulated in a complex way. On the other hand, studies on transgenic animals are very complicated and laborious.

Grimm and Leder (J. Exp. Med. 185 (1997), 1137-1142) describe a method for identifying and isolating dominant apoptosis-inducing nucleic acid sequences. Here, small plasmid pools corresponding to 20 clones from normalized cDNA expression libraries are transiently introduced into the human kidney cell line 293. The apoptosis-inducing activity of a nucleic acid sequence is determined manually by microscopic inspection of morphological features characteristic for apoptosis. With the aid of this method, it was possible to identify the apoptosis-inducing gene of adenine-nucleotide translocase-1 (ANT-1). The ANT-1 gene is assumed to be the cause of the degenerative cardiac disorder dilated cardiomyopathy (DCM) (PCT/EP00/08812).

The method described uses plasmid pools of 20 clones for transfection into the kidney cell line 293. However, the transfection of plasmid pools is able to achieve only a low sensitivity for the screening method, since those expression plasmids which contain apoptosis-inducing genes are in each case present at a low ratio to the expression plasmids not carrying apoptosis-inducing genes. In order to avoid this problem, instead of plasmid pools the screening method employed here uses, for transfection, aliquots which statistically contained just one clone. In addition, a particularly effective way of purifying plasmid DNA was used (Neudecker and Grimm, Biotechniques 28 (2000), 107-109). This method makes it possible to reduce lipopolysaccharides by a factor of 900 compared to previously published methods and thus makes it possible, for the first time, to purify plasmid DNA for high throughput in the 96-well format. Due to using specially purified DNA for transfection combined with using individual clones instead of plasmid pools, it was possible to carry out a particularly effective and sensitive screening method which made it possible to determine a large number of apoptosis-inducing sequences.

The present invention relates to novel apoptosis-associated and in particular apoptosis-inducing nucleic acids comprising:

(a) the nucleic acids of clones 1-146, shown in table 1, in table 2 or in SEQ ID NO: 1-225, nucleic acids complementary thereto or fragments thereof,

(b) nucleic acids corresponding to the sequences as claimed in (a) within the framework of the degeneracy of the genetic code and

(c) nucleic acids hybridizing with the sequences as claimed in (a) or/and (b) under stringent conditions.

The nucleic acids of the invention are apoptosis-associated nucleic acids, i.e. nucleic acids associated with the appearance of apoptotic processes in a cell, in particular in a mammalian cell. The nucleic acids are preferably apoptosis-inducing nucleic acids, i.e. nucleic acids which can cause or/and promote apoptotic processes. The nucleic acids of the invention are particularly preferably dominant apoptosis-inducing nucleic acids which are capable of, when expressed in a cell, inducing apoptosis and of causing the features characteristic for apoptosis, such as, for example, DNA fragmentation, unusual morphological features, etc. The nucleic acids may be present in double-strand or single-strand form, for example as DNA or RNA. The isolated nucleic acids can exhibit their cellular effect by expression, in particular by overexpression in cells. As a consequence, they are inducible and their use as therapeutic agent is specified.

Besides the nucleic acids shown in table 1, the sequence listings or fragments thereof having a length of preferably at least 15, particularly preferably at least 20 and most preferably at least 25 nucleotides, the present invention also includes variants of said sequences. Besides the nucleic acids which correspond to the sequences according to (a) within the framework of the degeneracy of the genetic code and which code for a polypeptide with an identical amino acid sequence, nucleic acids which hybridize with the sequences according to (a) or/and (b) under stringent conditions are also included. Hybridization under stringent conditions means within the scope of the present application that a hybridization signal is still found after prehybridization and hybridization under suitable conditions and washing in 1×SSC and 0.1% SDS at 55° C., preferably at 62° C. and particularly preferably at 68° C. and, in particular, in 0.2×SSC and 0.1% SDS at 55° C., preferably at 62° C. and particularly preferably at 68° C. (see also Sambrook et al., Molecular Cloning. A Laboratory Manual (1989), Cold Spring Harbor Laboratory Press, 1.101-1.104).

The apoptosis-associated nucleic acids of the invention preferably code for an apoptosis-associated polypeptide or a functional fragment thereof. The nucleic acids may come from any organism, with eukaryotic organisms such as nematodes, e.g. C. elegans, arthropods such as Drosophila, cordata and vertebrates, e.g. mammals, are preferred. Particular preference is given to sequences of mammals, for example of mice or humans, and said sequences can where appropriate also be altered by known molecular-biological techniques such as, for example, site-specific mutagenesis, PCR, restriction cleavage and ligation.

The nucleic acids of the invention are preferably present operatively linked to an expression control sequence so that they can be transcribed and, where appropriate, translated in a suitable host cell. Expression control sequences usually include a promoter and, where appropriate, regulatory sequences such as operators or enhancers. Furthermore, translation initiation sequences may also be present. Suitable expression control sequences for prokaryotic or eukaryotic host cells are known to the skilled worker (see, for example, Sambrook et al., supra).

The invention further relates to a recombinant vector containing a nucleic acid of the invention, preferably operatively linked to an expression control sequence. Furthermore, the recombinant vector may also contain conventional elements such as an origin of replication and a selection, marker gene. Examples of suitable recombinant vectors, for example, plasmids, cosmids, phages, viruses, etc., are known to the skilled worker (see, for example, Sambrook et al., supra).

The invention still further relates to recombinant cells which may have been transformed or transfected with a nucleic acid of the invention or a vector of the invention. Transformation or transfection may be carried out according to known methods, for example by calcium phosphate coprecipitation, lipofection, electroporation, particle bombardment or viral infection. The cell of the invention may contain the recombinant nucleic acid in extrachromosomal or chromosomally integrated form.

The invention still further relates to apoptosis-associated polypeptides encoded by a nucleic acid of the invention. Apoptosis-associated polypeptides may be obtained by expressing the apoptosis-associated nucleic acids of the invention, by chemical synthesis or by combinations of the two methods.

The invention still further relates to a pharmaceutical composition containing a nucleic acid of the invention, a vector of the invention or a polypeptide of the invention, where appropriate together with pharmaceutically conventional carriers and excipients. The nucleic acids, vectors, cells and polypeptides described above may be used for producing a diagnostic or therapeutic agent, in particular an agent for the diagnosis, therapy or prevention of apoptosis-associated disorders. Apoptosis-associated disorders may be distinguished on the one hand by abnormally reduced apoptosis and thus by hyperproliferation, for example tumor disorders, autoimmune diseases and viral infections (Thompson, Science 267 (1995), 1456-1462). On the other hand, apoptosis-associated disorders may also be distinguished by an abnormal increase in apoptosis and thus by degenerative symptoms, such as, for example, Alzheimer's disease, Huntington's disease, Parkinson's disease, reperfusion damage, stroke, and liver damage due to alcohol (Thompson (1995), supra).

The diagnostic application includes qualitative or/and quantitative detection of the apoptosis-associated nucleic acid, for example in the form of a transcript, or of the polypeptide encoded thereby in a sample, in particular a sample removed from a diseased organism, for example a patient. Detection may be carried out in the conventional manner, for example by nucleic acid hybridization or nucleic acid amplification reactions such as, for example, PCR or by protein detection via antibodies. Numerous techniques for these purposes are known to the skilled worker. Detection may also be carried out by using the isolated genes on a DNA chip. As a result, it is possible to study a plurality of genes, for example all genes, simultaneously in one experiment.

The therapeutic or preventive application includes administration of an active substance to a diseased organism in a sufficient dosage in order to alleviate or cure the apoptosis-associated disorder or to prevent the onset of an apoptosis-associated disease. In one embodiment of the invention, an apoptosis-associated nucleic acid is administered on a vector for gene therapy, for example an adenovirus, a retrovirus, an adeno-associated virus, etc., in order to effect increased expression of the apoptosis-associated nucleic acid in a diseased target cell. As an alternative it is also possible to administer an antisense nucleic acid, for example on a vector for gene therapy or else directly, if a reduction in apoptosis-associated nucleic acid expression is planned. In a further embodiment of the invention it is possible to administer apoptosis-associated poly-peptides or modulators of the activity of such apoptosis-associated polypeptides, for example activators or inhibitors. The active substances are administered according to known methods such as those, for example, described for gene therapy (Anderson, Nature 392 (1998), 25-30) or protein therapy (Schwarze et al., Science 285 (1999), 1569-1572).

Finally, it is also possible to use the nucleic acids, vectors, cells and polypeptides of the invention for identifying novel active substances for the therapy or prevention of apoptosis-associated disorders. A possibility here is the use in known cellular or molecular screening assays, where appropriate in a high-throughput format. The invention relates of course also to the active substances or substances derived therefrom which are identified by applying such screening methods. The active substances identified by the screening are capable of activating or inhibiting signal pathways induced by expression of the nucleic acids.

The present invention further relates to transgenic nonhuman animals which (i) constitutively or inducibly overexpress the gene of a nucleic acid of the invention or the ANT-1 gene, (ii) contain the endogenous gene of a nucleic acid of the invention or the ANT-1 gene in an inactivated form, (iii) contain the endogenous gene of a nucleic acid of the invention or the ANT-1 gene, which has been replaced completely or partially by a mutated gene of a nucleic acid of the invention or by a mutated ANT-1 gene, (iv) overexpress or underexpress conditionally and tissue-specifically the gene of a nucleic acid of the invention or the ANT-1 gene, or (v) have a conditional and tissue-specific knockout of the gene of a nucleic acid of the invention or of the ANT-1 gene.

Preferably, the transgenic animal may also contain an exogenous gene of a nucleic acid of the invention or an exogenous ANT-1 gene under the control of a promoter allowing overexpression. Alternatively, the endogenous gene of a nucleic acid of the invention or the endogenous ANT-1 gene may be overexpressed by activating or/and replacing its own promoter. The endogenous promoter of the gene of a nucleic acid of the invention or of the ANT-1 gene has preferably a genetic modification which modifies expression of said gene. Said genetic modification of the endogenous promoter comprises both a mutation of individual bases and deletion and insertion mutations.

A first embodiment relates to a transgenic animal overexpressing constitutively or inducibly the gene of a nucleic acid of the invention or the ANT-1 gene. Where appropriate, the introduced gene of a nucleic acid of the invention or the introduced ANT-1 gene may have additional mutations.

A second embodiment relates to a transgenic animal which contains the endogenous gene of a nucleic acid of the invention or the endogenous ANT-1 gene in an inactivated form. In this connection, the gene of a nucleic acid of the invention or the ANT-1 gene is preferably inactivated by introducing a knockout mutation by means of homologous recombination or by introducing an antisense construct or an RNAi construct.

A third embodiment relates to a transgenic animal in which the endogenous gene of a nucleic acid of the invention or the endogenous ANT-1 gene has been replaced completely or partially by a mutated gene of a nucleic acid of the invention or by a mutated ANT-1 gene.

A fourth embodiment relates to a transgenic animal which overexpresses or underexpresses conditionally and tissue-specifically the gene of a nucleic acid of the invention or the ANT-1 gene.

In a fifth embodiment, the transgenic animal has a conditional and tissue-specific knockout of the gene of a nucleic acid of the invention or of the ANT-1 gene.

The transgenic animal is preferably a mammal such as, for example, a rodent, e.g. a mouse. Mice have numerous advantages compared with other animals. They can be kept easily and their physiology is regarded as a model system for that of humans. The skilled worker is sufficiently familiar with the preparation of genetically manipulated animals of this kind, which is carried out according to customary methods (Hogan, B., Beddington, R., Costantini, F. and Lacy, E. (1994), Manipulating the Mouse-Embryo; A Laboratory Manual, 2nd edition, Cold Spring Harbor Laboratory, Cold Spring Harbor, N.Y.).

Furthermore, the invention relates to the use of a transgenic animal of this kind for the genetic and/or pharmacological investigation of disorders connected with excessive or reduced expression or the lack of expression of a gene of a nucleic acid of the invention or of an ANT-1 gene.

The transgenic animals of the invention may serve as a model for the disorders linked to the gene of a nucleic acid of the invention or to the ANT-1 gene in humans or else in agricultural livestock. Thus it is possible, for example, to determine the effect of active substances or gene therapies on the course of the disease. The animals may likewise be useful for the diagnosis or early detection of a disease.

Thus, for example, an inventive transgenic animal containing the ANT-1 gene may serve as a model for the degenerative cardiac disorder dilated cardiomyopathy (DCM). This degenerative cardiac disorder is linked to excessive apoptosis in the cells of a patient's heart. A first indication of the apoptosis inducer ANT-1 playing an important part in the induction of apoptosis in DCM was the observation that, during the course of DCM, a shift in the expression pattern of ANT-1 isoforms in the heart of a patient occurs already at a very early stage, resulting in increased expression of ANT-1 mRNA and ANT-1 protein (PCT/EP00/08812). For this purpose, the ANT-1 gene can be expressed in transgenic mice under the control of the cardiac-specific α-myosin heavy chain promoter (Subramaniam, A. (1991), J. Biol. Chem. 266 (36), pages 24613-24620). This promoter has been well characterized and is switched on only at birth. The expression construct may be prepared, for example, by inserting the ANT-1 gene into the SalI restriction cleavage site of the third noncoding exon of the 5.5 kb promoter. The hearts of the inventive transgenic animals prepared in this way should have some of the DCM-specific cellular modifications such as fibrination, apoptosis and hypertrophy or dysfunctions such as left-ventricular pressure, end-diastolic pressure, contractility, left-ventricular ejection fraction and left-ventricular filling pressure.

As an alternative or in addition, it is also possible to use cell culture systems, in particular human cell culture systems for the applications described above for the transgenic animal.

Furthermore, the following example is intended to illustrate the invention in more detail.

The sequence listing contains the sequences SEQ ID No. 1-225 which comprise the T7 sequences, BGH sequences and internal primer sequences of the identified apoptosis-inducing genes of clones 1-124, listed in table 1. Table 2 shows the sequences of clones 1-124 and 125, 127, 133, 134, 140, 141, 144, 145 and 146.

### EXAMPLE

Isolation of Apoptosis-inducing Genes

1. General Remarks

Apoptosis-inducing genes were found by a genetic screening in the human cell line HEK 293T (Grimm and Leder (1997), supra), which is based on iterative transfection of small expression plasmid pools from a normalized gene library and subsequent microscopic determination of the programmed cell death by way of the phenotype of the apoptotic cells. The transfection of single clones from a positive plasmid pool then allows determination of the apoptosis-inducing gene.

Said screening was carried out in a 96-well format. Moreover, a particularly effective way of purifying the plasmid DNA was used (Neudecker and Grimm, Biotechniques 28 (2000), 107-109).

2. Experimental Protocols

2.1 Cell Culture and Transfections

Human HEK 293T cells were cultured in DMEM supplemented with 5% fetal calf serum (Sigma, Deisenhofen, Germany) in a humidified 5% CO₂ atmosphere. For transfections, the cells were introduced into 24-well plates and transfected with 2 μg of plasmid DNA according to the calcium phosphate coprecipitation method as described by Roussel et al. (Mol. Cell. Biol. 4 (1984), 1999-2009). For this, 25 μl of DNA solution were mixed with 25 μl of 2×HBS buffer pH 6.9 (274 mM NaCl, 10 mM KCl, 40 mM Hepes, 1.4 mM Na₂HPO₄) at 4° C. in a 96-well plate using a 12-channel pipetting machine (Eppendorf, Hamburg, Germany). After adding 20 μl of a 0.25 M CaCl₂ solution (4° C.) and mixing, 38 μl were applied to the cells, after incubation at room temperature for 25 min.

2.2 Generation of a Normalized Library and cDNA Screening

The normalization and construction of a kidney cDNA library were carried out as described by Grimm and Leder (J. Exp. Meth. 185 (1997), 1137-1142) and Sasaki et al. (Nucleic Acids Res. 22 (1994), 987-992).

mRNA from the kidney of 10-week-old CD1 mice was normalized by associating abundant mRNA species to antisense cDNA molecules covalently coupled to latex beads and subsequent removal by centrifugation. After two hybridization cycles, 200 ng (of 2 μg originally) of mRNA were obtained and used for preparing a cDNA library using a cDNA synthesis kit (Gibco BRL, Gaithersburg, Md.). After ligation of a BstXI adapter (Invitrogen, San Diego, Calif.) and cleavage with NotI, the cDNA molecules were inserted into a modified pcDNA3 vector (Invitrogen) under the control of the cytomegalovirus (CMV) promoter in which the neomycin resistance gene had been deleted. The DNA was introduced by electroporation into E. coli SURE cells (Stratagene Corp. La Jolla, Calif.) which were frozen immediately thereafter.

By plating out aliquots of the transformation mixture onto agar, it was found that the library contained about 2.5×10⁵ clones. Aliquots which statistically contained single clones were inoculated in 900 μl of LB medium in wells of 96-well blocks (Qiagen, Hilden, Germany) and cultured with shaking at 300 rpm for 30 h. The remaining DNA was used to transform bacteria for large-scale isolation of plasmids and to sequence the inserted DNA. On the basis of the DNA sequence, a sequence comparison with commercial sequence databases was carried out with the aid of the Blast computer program.

2.3 Determination of Apoptosis-inducing Nucleic Acids

The apoptosis-inducing activity of the nucleic acids transfected was determined by microscopic determination of the cell phenotype. The optical density of apoptotic cells increases, since the cytoplasm/nucleus volume ratio decreases and holes develop in the cytoplasma membrane due to degradation of the cytoskeleton.

2.4 Plasmid Isolation

96-well blocks with bacteria were centrifuged at 3 000 g for 5 min (Sigma centrifuges, Osterode am Harz, Germany). The supernatant was decanted and the blocks were inverted for 2 to 3 min. Then 170 μl of buffer P1 (50 mM Tris-HCl/10 mM EDTA pH 8.0) were added and the bacteria pellets were resuspended by complete vortexing for 10 to 20 min. After addition of 170 μl of buffer P2 (200 mM NaOH, 1% SDS), the block was sealed with foil, and the mixture was mixed by inverting and incubated at room temperature for 5 min. Lysis was stopped by adding 170 μl of 4° C. cold buffer P3 (3 M potassium acetate pH 5.5). Then 10 μl of RNaseA solution (1.7 mg/ml) were added, followed by incubation at room temperature and then at −20° C. for 5 min and another centrifugation at 6 000 rpm for 10 min. The supernatant was decanted into new blocks and 100 μl of buffer P4 (2.5% SDS in isopropanol) were added. The block was vortexed for 5 min followed by incubation initially at 4° C. for 15 min and then at −20° C. for 15 min.

After centrifugation at 6 000 rpm for 10 min, the supernatant was introduced into 96-well polyoxy-methylene microtiter blocks. 150 μl of a silicon oxide suspension were added followed by incubation at room temperature for 20 min. The plates were centrifuged at 6 000 rpm for 5 min. The supernatant was carefully decanted and 400 μl of acetone (−20° C.) were added. The plates were again vortexed (30 sec) and centrifuged at 6 000 rpm for 3 min. This acetone washing was repeated once. The plates were dried initially at room temperature for 5 min and then in a vacuum chamber for 5 min. The pellets were resuspended in 75 μl of water (60° C.) and centrifuged at 6 000 rpm and 4° C. for 10 min. The supernatant was stored in a 96-well microtiter plate at −20° C.

3. Results

Clone 1 is composed of the following sequences: T7 sequence, BGH sequence, internal primer left and internal primer right (sequences 1-4). The total sequence is listed under Contig (sequence 5). Clone 1 is homologous to the following ESTs (expressed sequence tags): uc81e12.y1 Sugano mouse kidney mkia Mus musculus cDNA clone (GenBank Acc. No AA986577.1|AA986577), 99% identity; mq28c01.r1 Barstead MPLRB1 Mus musculus cDNA clone (GenBank Acc. No AA137597.1|AA137597, 100% identity; vc94d05.r1 Barstead MPLRB1 Mus musculus cDNA clone (GenBank Acc. No AA388338.1|AA388338), 99% identity; vg82f08.r1 Barstead MPLRB1 Mus musculus cDNA clone (GenBank Acc. No AI098009.1|AI098009), 99% identity; uc81f01.x1 Sugano mouse kidney mkia Mus musculus cDNA clone (GenBank Acc. No AA986094.1|AA986094), 100% identity.

Clone 2 is composed of T7 sequence and BGH sequence. The total sequence is listed under Contig. Clone 2 is homologous to the following database entries: Homo sapiens cDNA FLJ20625 fis, clone KAT04008 (DNA Data Bank of Japan (DDBJ) Acc. No AK000632.1|AK000632), 91% identity; mf71g09.r1 Soares mouse embryo NbME13.5 14.5 Mus musculus cDNA clone (GenBank Acc. No W89554.1|W89554), 99% identity; G0103H12-3 Mouse E7.5 Embryonic Portion cDNA Library Mus musculus cDNA clone (GenBank Acc. No AW536394.1|AW536394), 99% identity.

Clone 3 is composed of T7 sequence and BGH sequence. The total sequence is listed under Contig. Clone 3 is homologous to EMBL Acc. No X78936|MMPHRPR: M. musculus mRNA for parathyroid hormone/parathyroid hormone related peptide receptor, 94% identity.

Clone 4 is composed of T7 sequence and BGH sequence. The total sequence is listed under Contig. Clone 4 is homologous to Mus musculus mRNA for calcium channel gamma 5 subunit (CACNG5 gene) (EMBL. Acc. No AJ272046.1|MMU272046), 97% identity.

Clone 5 is homologous to RefSeq Database Acc. No NM_{—}008218.1: Mus musculus hemoglobin alpha, adult chain 1 (Hba-a1), mRNA having a length of 564 bp and 97% identity, and GenBank Acc. No L75940.1|MUSALGL: Mus musculus alpha-globin mRNA, complete cds, 98% identity.

Clone 6 is composed of T7 sequence or R2 sequence and BGH sequence or 4SP6 sequence. The total sequence is listed under Contig. Clone 6 is homologous to the following database entries: Mus musculus domesticus mitochondrial carrier homolog 1 isoform a mRNA, complete cds; nuclear gene for mitochondrial (GenBank Acc. No AF176007.2|AF176007), 99% identity; Homo sapiens CGI-64 protein mRNA, complete cds: (GenBank Acc. No AF151822.1|AF151822), 90% identity; Homo sapiens presenilin-associated protein mRNA, complete cds (GenBank Acc. No AF189289.1|AF189289), 89% identity.

Clone 7 is homologous to GenBank Acc. No AF151893.1|AF151893: Homo sapiens CGI-135 protein mRNA, complete cds, 86% identity.

Clone 8 is homologous to RefSeq Acc. No NM_{—}012504.1: Rattus norvegicus ATPase, Na+K+transporting, alpha 1 polypeptide (Atp1a1), mRNA, 94% identity.

Clone 9 is homologous to the following database entries: Mus musculus solute carrier family 27 (fatty acid transporter), member 2 (Slc27a2), mRNA (RefSeq Acc. No NM_{—}011978.1), 99% identity; Mus musculus fatty acid transport protein 2 mRNA, complete cds (GenBank Acc. No AF072757.1|AF072757), 99% identity; Mus musculus mRNA for very-long-chain acyl-CoA synthetase (VLACS) (EMBL Acc. No AJ223958.1), 99% identity.

Clone 10 is composed of T7 sequence and BGH sequence. The total sequence is listed under Contig. Clone 10 is homologous to EMBL Acc. No AL080066.1|HSM800567: Homo sapiens mRNA; cDNA DKFZp564J142 (from clone DKFZp564J142), 89-95% identity, and to DDBJ Acc. No AK001993.1|AK001993: Homo sapiens cDNA FLJ11131 fis, clone PLACE1006325, highly similar to Homo sapiens mRNA; cDNA DKFZp564J142, 89-95% identity.

Clone 11 is homologous to the following database entries: RefSeq Acc, No NM_{—}009984.1: Mus musculus cathepsin L (Ctsl), mRNA, 96% identity; EMBL Acc. No X06086.1|MMMEPR: Mouse mRNA for major excreted protein (MEP), 96% identity; GenBank Acc. No J02583.1|MUSCPR: 96% identity.

Clone 12 is composed of T7 sequence and BGH sequence. The total sequence is listed under Contig. Clone 12 is homologous to the following database entries: EMBL Acc. No AL137721.1|HSM802233: Homo sapiens mRNA; cDNA DKFZp761H221, 89% identity; GenBank Acc. No L34839.1|HUMTUM: Homo sapiens over-expressed breast tumor protein mRNA, 89% identity; GenBank Acc. No AF216754.1|AF216754: Homo sapiens over-expressed breast tumor protein (OBTP) mRNA, complete cds, 89% identity; GenBank Acc. No U16813|HSU16813: Human Bak-3 pseudogene, complete cds, 88% identity. Clone 12 is homologous to the following ESTs: DDBJ Acc. No C88895|C88895: Mus musculus early blastocyst cDNA, clone 01B00051IK19, 97% identity, and GenBank Acc. No AA2687191|AA268719: Soares mouse NML Mus musculus cDNA clone 747619 5′, 97% identity.

Clone 13 is composed of T7 sequence and BGH sequence. The total sequence is listed under Contig. Clone 13 is homologous to GenBank Acc. No AF151807.1|AF151807: Homo sapiens CGI-49 protein mRNA, complete cds, 87-90% identity.

Clone 14 is homologous to GenBank Acc. No AF080469|AF080469: Mus musculus putative glycogen storage disease type 1b protein mRNA, complete cds, 94% identity.

Clone 15 shows identity with GenBank Acc. No U31241|CGU31241: Cricetulus griseus integral membrane protein CII-3 mRNA, nuclear gene encoding mitochondrial protein, complete cds, 85% identity, and with GenBank Acc. No S74803|S74803: CII-3 succinate ubiquinone oxidoreductase complex II membrane-intrinsic subunit [cattle, heart, mRNA, 83% identity.

Clone 16 is composed of T7 sequence or 4SP6 sequence and BGH sequence. The total sequence is listed under Contig. Clone 16 is homologous to GenBank Acc. No AF116911.1|AF116911: Mus musculus thymic dendritic cell-derived factor 1 mRNA, complete cds, 96% identity and to RefSeq Acc. No NM_{—}004872.1: Homo sapiens mouse tropomyosin homolog (HSPC001) mRNA, 89-92% identity.

Clone 17 shows identity with GenBank Acc. No AF056031|AF056031: Rattus norvegicus kynurenine 3-hydroxylase mRNA, complete cds, 90% identity, and with EMBL Acc. No Y13153|HSKYNU3MO: Homo sapiens mRNA for kynurenine 3-monooxygenase, 82% identity.

Clone 18 is composed of T7 sequence and BGH sequence. The total sequence is listed under Contig. Clone 18 is homologous to GenBank Acc. No AW109849.1|AW109849: MT2475 mouse liver, dioxin treated Mus musculus cDNA clone MT2475 3′, 94% identity; GenBank Acc. No AA277327.1|AA277327: va81e12.r1 Soares mouse NML Mus musculus cDNA clone IMAGE:737806 5′ similar to WP:C02F5.3 0. CE00039 GTP-BINDING PROTEIN, 93% identity; GenBank Acc. No AI646762.1|AI646762: ub65e0.1.×1 Soares_mammary_gland_NMLMG Mus musculus cDNA clone IMAGE:1382616 3′ similar to WP:C41D11.5 CE08662 ENDONUCLEASE, 94% identity.

Clone 19 is homologous to DDBJ Acc. No AB005451|AB005451: Mus musculus mRNA for RST, complete cds, 92% identity.

Clone 20 is homologous to GenBank Acc. No AA1090181|AA109018; mp37f03.r1 Barstead MPLRB1 Mus musculus cDNA clone 571421 5′ similar to SW:ACY2_HUMAN P45381 ASPARTOCYLASE, 96% identity.

Clone 21 is homologous to the following database entries: GenBank Acc. No M12673|RATGNPAS: RATGNPAS Rat guanine nucleotide-binding protein G-s, alpha subunit mRNA complete cds, 87% identity, and to EMBL Acc. No Y00703|MMGTPAMU: Mouse uncoupled S49 cells mRNA for stimulatory GTP-binding protein alpha subunit, 87% identity; GenBank Acc. No M17525|RATBPGTPD: RATBPGTPD Rat GTP-binding protein (G-alpha-8) mRNA, complete cds, 87% identity; GenBank Acc. No AF116268|AF116268: Mus musculus G-protein XLAS (Xlas) mRNA, alternatively spliced, complete cds, 94% identity.

Clone 22 is homologous to the following database entries: GenBank Acc. No AF061026|AF061026: Mus musculus leucine zipper-EF-hand containing transmembrane protein 1 (Letm1) mRNA, complete cds, 93-95% identity.

Clone 23 is homologous to the following database entries: GenBank Acc. No AA086895|AA086895: mk19c02.r1 Soares mouse p3NMF19.5 Mus musculus cDNA clone 493346 5′ similar to WP:F17C11.8 CE05655 and to GenBank Acc. No AA881548|AA881548: vx20b03.r1 Soares 2NbMT Mus musculus cDNA clone 1264973 5′, 88% identity, and to GenBank Acc. No AA073042|AA073042: mm78b04.r1 Stratagene mouse embryonic carcinomaRA (#937318) Mus musculus cDNA clone 534511 5′ similar to WP:F17C11.8, 89% identity.

Clone 24 is homologous to GenBank Acc. No U22465|MMU22465: Mus musculus Na/Pi-cotransporter (NaPi-7) mRNA, complete cds, 93% identity, and to GenBank Acc. No L33878|MUSSPT: Mus musculus renal sodium phosphate (Na+/Pi) transporter mRNA complete cds, 93% identity.

Clone 25 is homologous to GenBank Acc. No AF049882: Rattus norvegicus metastasis suppressor homolog (KAI1) mRNA, complete cds and to DDBJ Acc. No D14883|MUSC33R2IA: Mouse mRNA for C33/R2/IA4, complete cds, 94% identity. It was shown for Clone 25 that C33-induced apoptosis is mediated by the induction of free radicals of oxygen which lead to proapoptotic activation of mitochondria. These free radicals of oxygen are not generated by the mitochondrial respiratory chain, since cells having a genetic defect in the respiratory chain still respond to C33 expression with apoptosis.

The proapoptotic activity of C33 is not dependent on substrate or cell-cell interaction, since C33 causes even cells in suspension to become apoptotic. The rapid kinetics of the induction of apoptosis by C33 also differs from the proapoptotic effects of C33 stated previously. Furthermore, it has been shown that an extracellular loop in C33 which seems to be responsible for substrate interaction is not needed for the induction of apoptosis.

Clone 26 is homologous to GenBank Acc. No U50987|BTU50987: Bos taurus succinate-ubiquinone reductase membrane anchor subunit precursor QPs3 mRNA, complete cds, 86% identity and to DDBJ AB006202: Homo sapiens mRNA for cytochrome b small subunit of complex II, complete cds, 82% identity.

Clone 27 is homologous to GenBank Acc. No M31775|MUSCYTB558: Mouse cytochrome beta-558 mRNA, 3′ end, 88% identity.

Clone 28 is homologous to GenBank Acc. No U76253|MMU76253: Mus musculus E25B protein mRNA, complete cds, 88% identity.

Clone 29 is homologous to EMBL Acc. No Z98200.8|HS111B22: Human DNA sequence from clone RP1-111B22 on chromosome 6q16-21 Contains a novel pseudogene, a pseudogene similar to ribosomal protein L3, ESTs, STSs, GSSs and CpG Islands, complete sequence [Homo sapiens], 85% identity, and to GenBank Acc. No AW489000.1|AW489000: UI-M-BH3-asd-a-10-0-UI.s1 NIH_BMAP_M_S4 Mus musculus cDNA clone UI-M-BH3-asd-a-10-0-UI 3′, 85% identity, and to GenBank Acc. No AW488401.1|AW488401: UI-M-BH3-art-b-02-0-UI.s1 NIH_BMAP_M_S4 Mus musculus cDNA clone UI-M-BH3-art-b-02-0-UI 3′, 98% identity.

Clone 30 is homologous to the following database entries: GenBank Acc. No AI115883|AI115883: ue96a12.y1 Sugano mouse embryo mewa Mus musculus cDNA clone 1498942 5′, mRNA sequence [Mus musculus], 97% identity; GenBank Acc. No AA052396|AA052396: mb67c07.r1 Soares mouse p3NMF19.5 Mus musculus cDNA clone 334476 5′, 96% identity; GenBank Acc. No AA777720|AA777720: zj06a08.s1 Soares fetal liver spleen 1NFLS S1 Homo sapiens cDNA clone 449462 3′, 91% identity; GenBank Acc. No AA960116|AA960116: ub54e08.s1 Soares mouse mammary gland NMLMG Mus musculus cDNA clone 1381574 3′, 91% identity.

Clone 31 is homologous to the following database entries: GenBank Acc. No AI315450|AI315450: uj46g05.y1 Sugano mouse liver mlia Mus musculus cDNA clone IMAGE:1923032 5′, mRNA sequence [Mus musculus], 89% identity; GenBank Acc. No AI019678|AI019678: ua92d11.r1 Soares mouse mammary gland NbMMG Mus musculus cDNA clone 1364949 5′, 89% identity.

Clone 32 is homologous to GenBank Acc. No U07159|MMU07159: Mus musculus medium-chain acyl-CoA dehydrogenase mRNA, complete cds, 94% identity.

Clone 33 is homologous to GenBank Acc. No M76131|MUSEF2: Mouse elongation factor 2 (ef-2) mRNA, 3′ end, 89% identity.

Clone 34 is homologous to GenBank Acc. No AF072757|AF072757: Mus musculus fatty acid transport protein 2 mRNA, complete cds, 96% identity.

Clone 35 is homologous to EMBL Acc. No X76453|RNHREV107: R.norvegicus (Sprague Dawley) H-rev107 mRNA, 90% identity, and to EMBL Acc. No X92814|HSHREV107: H.sapiens mRNA for rat HREV107-like protein, 85% identity.

Clone 36 is homologous to GenBank Acc. No AW107362.1|AW107362: um15a0.4.X1 Sugano mouse kidney mkia Mus musculus cDNA clone IMAGE:2192334 3′ similar to SW:ACY2_HUMAN P45381 ASPARTOACYLASE, 93% identity.

Clone 37 is homologous to the following database entries: GenBank Acc. No AI315920|AI315920: uj27f11.y1 Sugano mouse kidney mkia Mus musculus cDNA clone IMAGE:1921197 5′ similar to TR:Q14521 Q14521 GIANT LARVAE HOMOLOGUE; mRNA sequence [Mus musculus], 87% identity; GenBank Acc. No AI315072|AI315072: uj23e11.x1 Sugano mouse kidney mkia Mus musculus cDNA clone IMAGE:1920812 3′, mRNA sequence [Mus musculus], 94% identity; GenBank Acc. No AF109905|MMHC213L3: Mus musculus major histocompatibility locus class II regions Hsc70t gene, partial cds; smRNP, G7A, NG23, MutS homolog, CLCP, NG24, NG25, and NG26 genes, complete cds; and unknown genes, 82-96% identity; GenBank Acc. No AF110520|MMHC425018: Mus musculus major histocompatibility complex region NG27, NG28, RPS28, NADH oxidoreductase, NG29, KIFC1, Fas-binding protein, BING1, tapasin, RalGDS-like, KE2, BING4, beta 1,3-galactosyl transferase, and RPS18 genes, 82% identity.

Clone 38 is homologous to GenBank Acc. No S45663|S45663: SC2=synaptic glycoprotein [rats, brain, mRNA, 1178nt], 91% identity.

Clone 39 is homologous to GenBank Acc. No AA763399|AA763399: vw53h02.r1 Soares mouse mammary gland NMLMG Mus musculus cDNA clone 1247571 5′ similar to WP:F32D8.4 CE05783 LACTATE DEHYDROGENASE, 96% identity.

Clone 40 is homologous to GenBank Acc. No U00677|U00677: Mus musculus syntrophin-1 gene, complete cds, 92% identity.

Clone 41 is homologous to GenBank Acc. No AC003043|AC003043: Homo sapiens chromosome 17, clone HRPC1067M6, complete sequence [Homo sapiens], 87% identity, and to GenBank Acc. No AA109006|AA109006: ml63d04.r1 Stratagene mouse testis (#937308) Mus musculus cDNA, 95% identity, and to GenBank Acc. No AA985996|AA985996: Sugano mouse liver mlia Mus musculus cDNA clone 1431282 3′ similar to TR:O14589 O14589 SIMILARITY TO Q09461, 93% identity.

Clone 42 is homologous to GenBank Acc. No U30838|MMU30838: Mus musculus voltage dependent anion channel 2 mRNA, nuclear gene encoding mitochondrial protein, complete cds, 94% identity, and to GenBank Acc. No L08666|HUMPORIN: Homo sapiens porin (por) mRNA, complete cds and truncated cds, 94% identity.

Clone 43 is homologous to the following database entries: GenBank Acc. No M27071|MUSDIS2M1A: Mus musculus protein phosphatase type 1 (dis2m1) mRNA, complete cds, 92% identity; GenBank Acc. No U53456|MMU53456: Mus musculus protein phosphatase 1cgamma (PP1cgamma) mRNA, complete cds, 98-100% identity; EMBL Acc. No X56438|DMPP1A1: D.melanogaster PP1-alpha 96A gene for protein phosphatase 1, 80% identity; GenBank Acc. No M27067|EMEBIMG: Aspergillus nidulans phosphoprotein phosphatase 1 mRNA, complete cds, 80% identity; GenBank Acc. No M60215|MZEZMPP1: Z.mays protein phosphatase-1 (ZmPP1) mRNA, complete cds, 83% identity; GenBank Acc. No U00063|CELF56C9: Caenorhabditis elegans cosmid F56C9, 81-86% identity.

Clone 44 is homologous to GenBank Acc. No U52842|MMU52842: Mus musculus kidney-specific transport protein mRNA, complete cds, 99% identity, and to DDBJ Acc. No AB004559|AB004559: Rattus norvegicus mRNA for multispecific organic anion transporter, complete cds, 93% identity.

Clone 45 is homologous to the following database entries: GenBank Acc. No AF080252|AF080252: Mus musculus serine/threonine protein kinase 51PK(S) mRNA, complete cds, 94% identity; GenBank Acc. No AF080253|AF080253: Mus musculus serine/threonine protein kinase 51PK(L) mRNA, complete cds, 94% identity; DDBJ Acc. No AB000449|AB000449: Homo sapiens mRNA for VRK1, complete cds, 83% identity.

Clone 46 is homologous to the following database entries: GenBank Acc. No J048061MUSOSP: Mus musculus osteopontin mRNA, complete cds, 95% identity; EMBL Acc. No X13986|MMPONTIN: Mouse mRNA for minopontin, 96% identity; EMBL Acc. No X16151|MMETA1: Mouse mRNA for early T-lymphocyte activation 1 protein (ETa-1), 95% identity; GenBank Acc. No S78177|S78177:Eta-1/Op(Eta-1b)=early T-lymphcyte activator-1 [mice, C3H/HeJ, spleen, mRNA, 1087 nt], 98% identity.

Clone 47 is homologous to DDBJ Acc. No AB006451|AB006451: Rattus norvegicus mRNA for Tim23, complete cds, 94% identity, and to GenBank Acc. No AF030162|AF030162: Homo sapiens inner mitochondrial membrane translocase Tim23 (TIM23) mRNA, nuclear gene encoding mictochondrial protein complete cds, 89% identity.

Clone 48 is homologous to DDBJ Acc. No D38549|HUMHA1025A: Human mRNA for KIAA0068 gene, partial cds, 86% identity, and to GenBank Acc. No AF072697|AF072697: Mus musculus SHYC (Shyc) mRNA, complete cds, 96% identity.

Clone 49 is homologous to GenBank Acc. No U07971|RNU07971: Rattus norvegicus Sprague-Dawley L-arginine:glycine amidinotransferase mRNA, partial cds, 85% identity.

Clone 50 is homologous to DDBJ Acc. No AU035342|AU035342: Sugano mouse brain mncb Mus musculus cDNA clone MNCb-0343, mRNA sequence [Mus musculus], 87% identity, and to GenBank Acc. No AI286459|AI286459: ui77d03.y1 Sugano mouse liver mlia Mus musculus cDNA clone IMAGE:1888421 5′ similar to WP:T06D8.9 CE02330. mRNA sequence [Mus musculus], 87% identity.

Clone 51 is homologous to GenBank Acc. No M22998|MUSGLUTRN: MUSGLUTRN Mouse facilitated glucose transport protein mRNA, complete cds, 93% identity, and to GenBank Acc. No S77924|S77924: Glut-1=glucose transporter isoform 1 [mice, embryo, mRNA Partial, 321 nt], 93% identity.

Clone 52 is homologous to EMBL Acc. No AJ010953|HSA010953: Homo sapiens mRNA for putative Ca2+−transporting ATPase, partial, 79% identity.

Clone 53 is homologous to Acc. No U94593: Mus musculus uncoupling protein homolog (UCPH) mRNA.

Clone 54 is homologous to Acc. No M27315.1: Rattus norvegicus mitochondrial cytochrome c oxidase subunits I, II and III, and ATPase subunit 6 genes, complete cds.

Clone 55 is composed of T7 sequence and BGH sequence. The total sequence is listed under total sequence. Clone 12 is homologous to the following database entries: Acc. No AL080317.11: Human DNA sequence from clone RP5-1112D6 on chromosome 6q21-22.2. Contains the gene for a PUTATIVE novel protein similar to bacterial NARK (nitrite extrusion protein, nitrite facilitator), the 3′ end of the REV3L gene for REV3 (yeast homolog)-like, catalytic subunit of DNA polymerase zeta (EC 2.7.7.7, POLZ), ESTs, STSs, GSSs and a putative CpG island, complete sequence.

Clone 56 is homologous to Acc. No AW106096.1: um23a10.y1 Sugano mouse embryo mewa Mus musculus cDNA clone IMAGE:2225370 5′, mRNA sequence.

Clone 57 is homologous to Acc. No NM_{—}000210.1 Homo sapiens integrin, alpha 6 (ITGA6) mRNA.

Clone 58 is homologous to Acc. No AW106096.1: um23a10.y1 Sugano mouse embryo mewa Mus musculus cDNA clone IMAGE:2225370 5′ mRNA sequence.

Clone 59 is homologous to Acc. No NM_{—}007748.1: Mus musculus cytochrome c oxidase, subunit VI a, polypeptide 1 (Cox6a1), mRNA.

Clone 60 is homologous to Acc. No AC005403.1: Mus musculus clone UWGC:ma53a068 from 14D1-D2 (T-Cell Receptor Alpha Locus), complete sequence.

Clone 61 is homologous to Acc. No L07095.1: Mus domesticus strain NZB/B1NJ mitochondrion genome, complete sequence.

Clone 62 is homologous to Acc. No NM_{—}009155.1: Mus musculus selenoprotein P, plasma, 1 (Sepp1), mRNA complete cds.

Clone 63 is homologous to Acc. No J03297.1: Mouse ERp99 mRNA encoding an endoplasmic reticulum transmembrane protein.

Clone 64 is homologous to Acc. No AF047431.1: Homo sapiens AAPT1-like protein mRNA, partial cds.

Clone 65 is homologous to EMBL Acc. No U79287 Human clone 23867 mRNA sequence 1396 bp.

Clone 66 is homologous to Acc. No C88489, AA073437, AI048028 and to ESTs with DDBJ Acc. No C88489; GenBank Acc. No AA073437; AI048028.

Clone 67 is homologous to Acc. No AK001441: Homo sapiens cDNA FLJ10579 fis, clone NT2RP2003446 2251 bp mRNA and to ESTs with Acc. No AI663355 and AU080732.

Clone 68 is homologous to Acc. No AF005038: Homo sapiens Secretory Carrier Membrane Protein (SCAMP2) mRNA.

Clone 69 is homologous to Acc. No U44731: Mus musculus putative purine nucleotide binding protein mRNA.

Clone 70 is homologous to Acc. No U16818: Mus musculus UDP glucuronosyltransferase (UGT1-06) mRNA.

Clone 71 is homologous to Acc. No NM_{—}004256: Homo sapiens organic cationic transporter-like 3 (ORCTL3) mRNA.

Clone 72 is homologous to Acc. No AK000559: Homo sapiens cDNA FLJ0552 fis, clone KAT11732.

Clone 73 is homologous to Acc. No X89968: Rattus norvegicus mRNA for alpha-soluble NSF attachment protein.

Clone 74 is homologous to Acc. No D85137: Mouse mRNA for PP1gamma (protein phosphatase1gamma).

Clone 75 is homologous to Acc. No NM₁₃ 001089: Homo sapiens ATP-binding cassette, sub-family A (ABC1), member 3 (ABCA3) mRNA.

Clone 76 is homologous to mouse ESTs with Acc. No AI315969, AI930239, AI666299 and AV141103.

Clone 77 is homologous to Acc. No AB025405: Mus musculus mRNA for sid2895p (mouse microsomal signal peptidase)

Clone 78 is homologous to Acc. No AK000427. The clone is partially homologous to: Homo sapiens cDNA FLJ20420 fis, clone KAT02462.

Clone 79 is homologous to Acc. No NM_{—}009127: Mus musculus stearoyl-Coenzyme A desaturase 1 (Scd1)

Clone 80 is homologous to Acc. No U21049: Human DD96 mRNA (a gene selectively upregulated in human carcinomas).

Clone 81 is homologous to Acc. No AF070626 and AB020980: Homo sapiens clone 24483 unknown mRNA (AF070626), and Homo sapiens mRNA for putative membrane protein (AB020980).

Clone 82 is partially homologous to Acc. No U92989: Homo sapiens clone DT1P1E11 mRNA, CAG repeat region.

Clone 83 is homologous to Acc. No AF035208: Mus musculus putative v-SNARE Vti1b mRNA (soluble NSF attachment protein receptor).

Clone 84 is homologous to Acc. No NM_{—}012504: Rattus norvegicus ATPase, Na+K+transporting, alpha 1 polypeptide (Atp1a1), mRNA.

Clone 85 is homologous to Acc. No NM_{—}007749: Mus musculus cytochrome c oxidase subunit VIIc (Cox7c), mRNA.

Clone 86 is homologous to Acc. No AF079565: Mus musculus ubiquitin-specific protease UBP41 (Ubp41) mRNA.

Clone 87 is homologous to Acc. No AJ006341: Mus musculus mRNA for peroxisomal integral membrane protein PMP34.

Clone 88 is homologous to Acc. No AF223950: Mus musculus TIM22 preprotein translocase (Tim22) mRNA, complete cds; nuclear gene for mitochondrial product.

Clone 89 is homologous to Acc. No NM_{—}011479: Mus musculus serine palmitoyltransferase, long chain base subunit 2 (Spt1c2), mRNA.

Clone 90 is homologous to Acc. No M23984: Rat mitochondrial proton/phosphate symporter mRNA, complete cds and to Acc. No X60036: H.sapiens mRNA for mitochondrial phoshhate carrier protein.

Clone 91 is homologous to Acc. No AF113127: Homo sapiens S1R protein (S1R) mRNA, complete cds (human cowpox virus homolog).

Clone 92 is composed of T7 sequence and BGH sequence. The total sequence is shown as an assembly of a plurality of EST sequences (EST assembly) in Contig. Clone 92 is homologous to UniGene Mm.86545.

Clone 93 is homologous to human genomic DNA (PAC 69E11 on chromosome 1q23-24).

Clone 94 is homologous to Acc. No NM_{—}011512: Mus musculus surfeit gene 4 (Surf4), mRNA.

Clone 95 is composed of T7 sequence and BGH sequence. The total sequence is shown as an assembly of a plurality of EST sequences (EST assembly) under total cDNA EST cluster. Clone 95 is homologous to UniGene: Mm.27841 (mouse) and Hs.100132 (human).

Clone 96 is homologous to UniGene Hs.133494, Mm.31778 and Rn.11778.

Clone 97 is homologous to Acc. No AF192558: DEFINITION Mus musculus domesticus mitochondrial carrier homolog 1 isoform b (Mtch1) mRNA, complete cds; nuclear gene for mitochondrial product and to Acc. No AF192558/AF192559: Homo sapiens mitochondrial carrier homolog 1 isoform b (MTCH1) mRNA, partial cds; nuclear gene for mitochondrial product.

Clone 98 is homologous to Acc. No NM_{—}016783: Mus musculus progesterone receptor membrane component (Pgrmc-pending), mRNA.

Clone 99 is homologous to Acc. No NM₁₃ 003002: Homo sapiens succinate dehydrogenase complex, subunit D, integral membrane protein (SDHD) mRNA.

Clone 100 is homologous to Acc. No U95822: Human putative transmembrane GTPase mRNA, partial cds.

Clone 101 is homologous to Acc. No X99963: M.musculus rhoB gene.

Clone 102 is homologous to Acc. No AF161525: Homo sapiens HSPC177 mRNA, complete cds. The gene contains a BH3 domain which is present in many Bcl-2-like genes. Genes containing such “BH3-only” domains are proapoptotic and have the evolutively conserved function of inducing apoptosis. The experiments showed that CGI-135 can interact with members of the Bcl-2 gene family. The BH3 domain is required for efficient induction of apoptosis by CGI-135.

Clone 103 shows no homology to any known sequences.

Clone 104 is homologous to Unigene Acc. No AW682500.30119 Mm.30119|Mm.30119: Mus musculus cDNA, 91% identity.

Clone 105 is homologous to EMBL Acc. No X98475.1|MMVASP: M.musculus VASP gene, 90% identity.

Clone 106 is homologous to EMBL Acc. No X03369.1|RNTUBB15: Rat mRNA for beta-tubulin T beta15, 90% identity.

Clone 107 is homologous to GenBank Acc. No BE226644.1|BE226644: ia23h11.y1 Mouse E10 5 12 5 Pancreas cDNA Library Mus musculus cDNA 5′ similar to SW:NUML_{—BOVIN Q}01321 NADH-UBIQUINONE OXIDOREDUCTASE MLRQ SUBUNIT, 87% identity.

Clone 108 is homologous to GenBank Acc. No AF157317.1|AF157317: Homo sapiens AD-015 protein mRNA, complete cds, 83% identity. Clone 109 is homologous to GenBank Acc. No M88136.11CRUSTSTA: Cricetulus griseus seryl tRNA synthetase mRNA, partial cds, 91% identity.

Clone 110 shows no homology to any known sequences.

Clone 111 is homologous to GenBank Acc. No AW106096.27359|Mm.27359: um23a10.y1 Mus musculus cDNA, 5′ end, 99% identity.

Clone 112 is homologous to GenBank Acc. No AI413025.1|AI413025 EST241326 Normalized rat brain, Bento Soares Rattus sp. cDNA cloneRBRDZ28 3′ end, 97% identity.

Clone 113 is homologous to EMBL Acc. No Human DNA sequence from clone RP3-402G11 on chromosome 22q13.31-13.33 “Contains the MAPK12 gene for mitogen activated protein kinase 12 (SAPK3), the MAPK11 gene for mitogen activated protein kinase 11 (PRKM11), gene KIAA0315, the gene for a novel protein”, 86% identity.

Clone 114 is homologous to DDBJ Acc. No AK000823.1|AK000823 Homo sapiens cDNA FLJ20816 fis, clone ADSE00693, 88% identity.

Clone 115 is homologous to GenBank Acc. No AF007152.1|AF007152 Homo sapiens clone 23649 and 23755 unknown mRNA, partial cds, 83% identity.

Clone 116 is homologous to EMBL Acc. No X66370.1|RNRPS9 R.norvegicus mRNA for ribosomal protein S9, 94% identity.

Clone 117 is homologous to GenBank Acc. No BE161116.1|BE161116 PM3-HT0424-170200-001-b11 HT0424 Homo sapiens cDNA, 100% identity.

Clone 118 is homologous to DDBJ Acc. No AK000559.1|AK000559 Homo sapiens cDNA FLJ20552 fis, clone KAT11732, 86% identity.

Clone 119 is homologous to RefSeq Acc. No NM_{—}003713.1|Homo sapiens phosphatidic acid phosphatase type 2b (PPAP2B) mRNA, and translated products, 87% identity.

Clone 120 is homologous to GenBank Acc. No M30773.1 (HUMCNR: Human calcineurin B mRNA, complete cds, 88% identity.

Clone 121 is homologous to RefSeq Acc. No NM_{—}013770.1 or GenBank Acc. No AF188712.1 (AF188712: Mus musculus solute carrier family 25 (mitochondrial carrier; adenine nucleotide translocator), member 10 (Slc25a10), mRNA and Mus musculus mitochondrial dicarboxylate carrier mRNA, complete cds; nuclear gene for mitochondrial product, 98% identity.

Clone 122 is homologous to RefSeq Acc. No NM_{—}008525.1: Mus musculus delta-aminolevulinate dehydratase (Lv), mRNA, 100% identity.

Clone 123 is homologous to GenBank Acc. No M59861.1 (RAT10HCO: Rattus norvegicus 10 formyltetrahydrofolate dehydrogenase mRNA, complete cds, 93% identity.

Clone 124 is homologous to the following database entries: EMBL Acc. No X00525.1 (MmRNA02: Mouse 28S ribosomal RNA, 84% identity; GenBank Acc. No AF061799.1 (AF061799: Hydrolagus colliei internal transcribed spacer 1, partial sequence; 5.8S ribosomal RNA gene and internal transcribed spacer 2, complete sequence; and 28S ribosomal RNA gene, partial sequence, 83% identity; GenBank Acc. No AF061800.1 (AF061800: Squalus acanthias internal transcribed spacer 2; and 28S ribosomal RNA gene, partial sequence, 83% identity.

Clone 125 is homologous to RefSeq Acc. No NM_{—}007469.1: Mus musculus apolipoprotein CI (Apoc1), mRNA and AK011358.1|AK011358 Mus musculus 10 days embryo cDNA, RIKEN full-length enriched library, clone: 2610009E08, full insert sequence, 100% identity.

Clone 127 is homologous to GenBank Acc. No AK007676: Mus musculus 10 day old male pancreas cDNA, RIKEN full-length enriched library, clone: 1810032P22, full insert sequence, 100% identity; to GenBank Acc. No BC002242: Mus musculus, similar to sorting nexin 5, clone MGC:7534, mRNA, complete cds, 100% identity, and to GenBank Acc. No AC008066: Homo sapiens BAC clone RP11-293D9 from 8, complete sequence.

Clone 133 is homologous to GenBank Acc. No AK015068.1|AK015068: Mus musculus adult male testis cDNA, RIKEN full-length enriched library, clone: 4930403006, full insert sequence, 90% identity, and to DDBJ Acc. No AK004504.1|AK004504: Mus musculus 18 days embryo cDNA, RIKEN full-length enriched library, clone: 1190006A08, full insert sequence.

Clone 134 is homologous to GenBank Acc. No BC002108.1|BC002108: Mus musculus, Similar to ribosomal protein S27a, clone MGC:6601, mRNA, complete cds, 98% identity.

Clone 140 is homologous to DDBJ Acc. No AK008346.1|AK008346 Mus musculus adult male small intestine cDNA, RIKEN full-length enriched library, clone: 2010107K03, full insert sequence, 99% identity, and to RefSeq Acc. No NM_{—}007512.1 | Mus musculus ATPase inhibitor (Atpi), mRNA, 99% identity.

Clone 141 shows no homology to any known sequences.

Clone 144 is homologous to GenBank Acc. No AF006482.1|AF006482 Mus musculus nucleoside triphosphatase (NTPase) mRNA, complete cds, 99% identity; to GenBank Acc. No AF084569.1|AF084569 Mesocricetus auratus cph proto-oncogene product (cph) mRNA, complete cds, 90% identity.

Clone 145 is homologous to DDBJ Acc. No AK002787.1|AK002787 Mus musculus adult male kidney cDNA, RIKEN full-length enriched library, clone: 0610038B12, full insert sequence, 100% identity; to DDBJ Acc. No AK010989.1|AK010989 Mus musculus 13 days embryo liver cDNA, RIKEN full-length enriched library, clone: 2510022D06, full insert sequence, 100% identity; to RefSeq Acc. No NM_{—}031101.1| Rattus norvegicus ribosomal protein L13 (Rpll3), mRNA, 93% identity.

Clone 146 is homologous to RefSeq Acc. No NM_{—}025278.1| Mus musculus guanine nucleotide binding protein (G protein), gamma 12 (Gng12), mRNA, 99% identity.

The apoptosis-inducing genes (clones 1-124) identified by the genetic screening are listed in table 1 (pages 1-125) which is included as figure:

The following definitions relate to the information in table 1:

| | |
|---|---|
| “T7 sequence”: | 5′ sequence of the clone |
| “BGH sequence”: | 3′ sequence of the clone |
| “Internal Primer”: | internal sequences of the clone, |
| | identified using primers obtained |
| | from the T7 sequence (left) or the |
| | BGH sequence (right) |
| “Identity”: | comparison with sequences from the |
| | BLAST computer program (Altschul et |
| | al. (1997), Nucleic Acids Res. 25: |
| | 3389-3402). Apart from completely |
| | identical sequences (100% identity) |
| | partly identical sequences |
| | (preferably ≧ 85% identity) which |
| | show allelic variants of the |
| | actually shown sequence or |
| | homologous sequences from other |
| | species, in particular from humans, |
| | are also indicated. |
| | Variants or homologous sequences or |
| | this kind are, of course, likewise |
| | included in the present invention. |
| “Contig” | complete sequence; combined from |
| | the “T7 sequence”, “BGH sequence”, |
| | and, where appropriate, from the |
| | “internal primer” sequences. |
| “Properties” | information on predicted properties |
| | of the resulting peptide |
| “Prediction” | an analysis using the “PSORT II” |
| | computer program (Nakai, K. and |
| | Kanehisa, M., Genomics 14: 897-911 |
| | (1992)) determines, on the basis of |
| | the sequence, possible properties |
| | and also cellular location of the |
| | resulting peptide with a predicted |
| | probability. For clone 1, for |
| | example, two transmembrane domains |
| | and location in the ER (endoplasmic |
| | reticulum) with a probability of |
| | 33%, in the vacuole with a |
| | probability of 22%, in the |
| | mitochondria with a probability of |
| | 11%, and in the Golgi apparatus with |
| | a probability of 11%, are |
| | determined. |
| “DNA ladder” | biochemical method of detecting |
| | apoptosis (Bitzer et al. (1999) J. |
| | Virol. 73: 702-708) |
| “PI-Facs” | propidium iodide Facs: method of |
| | detecting apoptosis (Bitzer et al. |
| | (1999) J. Virol. 73: 702-708) |
| “possible ORF” | information on a possible “open |
| | reading frame” (ORF) in the one- |
| | letter code |

The apoptosis-induced genes (clones 1-124, 125, 127, 133, 134, 140, 141, 144, 145, 146) identified by the genetic screening are listed in table 2 (pages 1-28) which is included as figure.

TABLE 1

| | | | | |
|---|---|---|---|---|
| Clone # | T7 sequence | AGCTCATGTTGGACAGTCGTGTGAGGAGCTATGGAGCACAGCAGTAATCGCCCAGAGGACTTCCCGCTTAACGTGT | | |
| 1 | | TCTCTGTCACTCCGTACACACCCAGTACCGCCGACATCCAGGTGTCCGACGACGACAAGGCAGGGGCCACTTTGCTT | | |
| | | TTCTCAGGCATCTTTCTAGGACTGGTGGGGATCACTTTCACTGTCATGGGCTGGATCAAATACCAAGGTGTCTCCCA | | |
| | | CTTTGAATGGACCCAGCTCCTCGGACCCATCCTTCTGTCGGTCGGAGTGACATTCATCCTGATCGCTGTGTGCAAAT | | |
| | | TCAAAATGCTATCCTGCCAGTTGTGCTCAGATAACGAGGAGAGGGTCCCGGACTCGGACCAGACTTCCGGAGGACA | | |
| | | GTCGTTCGTTTTCACTGGCATCAATCAGCCCATCACCTTCCACGGGGCCACCGTGGTGCAGTATATCCCTCCTCCTT | | |
| | | ACGGTTCTCAGGAGCCCCTGGGAATGAACGCCACCTACCTGCAACCCATGATGAATCCTTGCGGTCTCATACCTCCT | | |
| | | AGTGGAGCAGCGGCTGCCGCACCAAGTCCCCCTCAGTACTACACCATCTACCCTCAAGACAATGCTGCGTTCGTNGA | | |
| | | GAGTGAGGGCTTCTCTCCTTTCGTGGGCACTGGATATGACAGGCCCGACTCTGATGCTGACCAGCTAGAAGGGACG | | |
| | | GAGTTGGAAGAGGAGGACTGCGTATGTTCTCTTCTCCACCGNATGAGGAGAATACGCTCTACCTCGCTNGAGACTG | | |
| | | CAANCTAAGGGACGGCATTTAAGCCCCTGNGATGNGAACTTGNGNGNNAACCNTGGGTCTTTANAAGTAGGNGNAA | | |
| | | ACN | | |
| | BGH sequence | ATAGACATGTCTAGTTTTTTATTACTAGTTATCATCCAAGTGAAATGTCCCTGAGGCATAATATGAATCACAATAATA | | |
| | | AACCAAGATTGTTTTGCTGATATCCTTGAAAAACCTGGACTCTTCTGACAGAGAGTAAAAGCAATCCCACATATAAG | | |
| | | TGATCTTTGTAAAACTGTTTTCTTTCTAATTCTTTGGACACCTCCTAAAGTCTGCAGCTTTGGGCTGNGGTTGGTGA | | |
| | | CACCGTTCAGACTTAGGAAGCTGAAGGCTACCAGCTTTTCTTTGTTCAAATGAAGGTGAGAGCTACGCCCGGNGGC | | |
| | | GGGNTAGANNGAGAC | | |
| | Internal primer | TTCCCACGCNGNTGNGGGCTTCTCTCCTTTCGTGGGCACTGGATATGACAGGCCCGACTGTGATGCTGACCAGCTA | | |
| | left | GAAGGGACGGAGTTGGAAGAGGAGGACTGCGTATGTTTCTCTCCTCCACCGTATGAGGAGATATACGCTCTACCTC | | |
| | | GCTAGAGACTGCAATGCTAAGGGGACGGACATTTAAGCCCTGNGATGTGATACTTGGAGAGTTTATCGCTGAGTTC | | |
| | | TTCAGAAGTTAGGTGTGAAAGCAGCTNAGGAGATCTTACAGATGTCATTNAAGGNGGGAAAGAAGTGCCCNGAGAC | | |
| | | TGCTAAATTAAGCTGCCCTGGTTAAATTCCCCTCTGCTCTGGTTTTGAATTCTCTCAGCTAAGAAACCCTCTGCAGC | | |
| | | TGGAGAGTCGCTCTGAGATAGAGAGATTTNGGAGCCCACGCAGNGCCTNGGGCTNGATCTCTAGAGCCAGAAGAAA | | |
| | | ACA | | |
| | Internal primer | TCCCAGCCTNTTCTNTGNTCAAATGAAGGTNAGAGCTACGCCCGGGGGCGGAGGGNGGGGGAGACGGGATGACTG | | |
| | right | ATCATCTCCATGATTCATGACTACAAAGCGACACGGCAGNATGGCTCTGNGGAACAGATCAGAGGCATACCTGTGTT | | |
| | | CCATAAGCNACTCAGCTGTACCTGCGTTCTGTAAGCCACTCGGCTGAGCTAGTTCGNGCCTGGGAATCACAGCCTG | | |
| | | GGGGNGGGCAGAGGGAGCAGGCGCTNACCNATGTACATTACGCCTTGAACTTCANCCTTGCAAAGGAAAACNATCA | | |
| | | NGAGCAGGGGTCACTNTAACTCGGNGGACAGCACATGGNGACATTCATTATCCTCCTGNTACCTCGTCCACAGTAGG | | |
| | | ATCGGCCACCCAACACTATTCTAACATGGGGCACTGTTTCATCCNACTCTACTTATTCATTTATTTCTTAATGGATTT | | |
| | | TATTTCTTTAACAAACTCTTNAAGATCCAAAGGCTTCCAGNGAGACCANATAANAGTCNAAGTTGTCTAANATAAGTN | | |
| | | ACTCTGCTGCGGAAGGTTCAGNGCCGTGCAGGGAAATTTCTAGCTGAGCCTGCTCTCTTCCCTGCTTGCTTGCCAG | | |
| | | CCTCCCTTTNATCANTCTNAGAGCTGCCATGGCTGCCCGGATTTAAAACTAANACAATCTTTNAAAACTAAGATNTCC | | |
| | | TTTAAATGGATATTCCGGATGG | | |
| | Contig | AATTCGGATCCATGCCCAAAGGTCCTTCTCTAACTAACCGGAGCATGGTGTTCCCCACTC | | |
| | | TCACACTCTGTTTCCTCTCATGTTGGACAGTCGTGTGAGGAGCTATGGAGCAC | | |
| | | AGCAGTAATCGCCCAGAGGACTTCCCGCTTAAGGTGTTCTCTGTCACTCCGTACACACCC | | |
| | | AGTACCGCCGACATCCAGGTGTCCGACGACGACAAGGCAGGGGCCACTTTGCTTTTCTCA | | |
| | | GGCATCTTTCTAGGACTGGTGGGGATCACTTTGACTGTCATGGGCTGGATCAAATACCAA | | |
| | | GGTGTCTCCCACTTTGAATGGACCCAGCTCCTCGGACCCATCCTTCTGTCGGTCGGAGTG | | |
| | | ACATTCATCCTGATCGCTGTGTGCAAATTCAAAATGCTATCCTGCCAGTTGTGCTCAG | | |
| | | ATAACGAGGAGAGGGTCCCGGACTCGGACCAGACTTCCGGAGGACAGTCGTTCGTTTTCA | | |
| | | CTGGCATCAATCAGCCCATCACCTTCCACGGGGCCACCGTGGTGCAGTATATCCCTCCT | | |
| | | CCTTACGGTTCTCAGGAGCCCCTGGGAATGAACGCCACCTACCTGCAACCCATGAT | | |
| | | GAATCCTTGCGGTCTCATACCTCCTAGTGGAGCAGCGGCTGCGCACCAAGTCCCCCTCAG | | |
| | | TACTACACCATCTACCCTCAAGACAATGCTGCGTTCGTGGAGAGTGAGGGCTTCTCTCCT | | |
| | | TTCGTGGGCACTGGATATGACAGGCCCGACTCTGATGCTGACCAGCTAGAAGGGACGGAG | | |
| | | TTGGAAGAGGAGGACTGCGTATGTTTCTCTCCTCCACCGTATGAGGAGATATACGCTCTA | | |
| | | CCTCGCTAGAGACTGCAATGCTAAGGGGACGGACATTTAAGCCCTGTGATGTGATACTTG | | |
| | | GAGAGTTTATCGCTGTGTTCTTCAGAAGTTAGGTGTCAAAGCAGCTCAGGAGATCTTACA | | |
| | | GATGTCATTCAAGGTGGGAAAGAAGTGGCCCGAGACTGCTAAATTAAGCTGCCCTGGTTA | | |
| | | AATTCCCCTCTGCTCTGGTTTTGAATTCTCTCAGCTAAGAAACCCTCTGCAGCTGGAGAG | | |
| | | TCGCTCTGTGATAGAGTGATTTTGGAGCCCACGCAGTGCCTTGGGTTTGATCTCTAGAG | | |
| | | CCAGAAGAAAACAAAAACAAAAACAAAAACAAAACAAGACCTCTCTACATAAA | | |
| | | GTGCAGGAGGAAAATTCACCCATTTCCCCATCCCCCACCCGATATCCATTTGAAGGATA | | |
| | | TCTTAGTTTTGAAAGATTGTCTTAGTTTTAAATCCGGCAGCCATGGCAGCTCTCAGACTG | | |
| | | ATGAAAGGGAGGCTGGCAAGCAAGCAGGGAAGAGAGCAGGCTCAGGTAGAAATTTCCCT | | |
| | | GCACGGCGCTGAACCTTCCGCAGCAGAGTGACTTATCTTAGACAACTTGGGCTGTTAT | | |
| | | CTGGTCTCCCTGGAAGCCTTTGGATCTTGAAGAGTTTGTAAAAGAAATAAAATCCATTA | | |
| | | AGAAATAAATGAATAAGTAGAGTGGGATGAAACAGTGCCCCATGTTAGAATAGTGTTGGG | | |
| | | TGGCCGATCCTACTGTGGACGAGGTAACAGGAGGATAATGAATGTCACCATGTGCTGTCC | | |
| | | ACCGAGTTACAGTGACCCCTGCTCCTGATGGTTTTCCTTTGCAAGGCTGAAGTTCAAGGC | | |
| | | GTAATGTACATGGGTGAGCGCCTGCTCCCTCTGCCCACCCCCAGGCTGTGATTCCCAGGC | | |
| | | ACGAACTAGCTCAGCCGAGTGGCTTACAGAACGCAGGTACAGCTGAGTGGCTTATGGAAC | | |
| | | ACAGGTATGCCTCTAATCTGTTCCACAGAGCCATGCTGCCGTGTCGCTTTGTAGTCATGA | | |
| | | ATCATGGAGATGATCAGTCATCCCGTCTCCCCCACCCCCCGCCGCCGGGCGTAGCTCT | | |
| | | CACCTTCATTTGAACAAAGAAAAGCTGGTAGCCTTCAGCTTCCTAAGTCTGAAGGGTGTC | | |
| | | ACCAACCACAGCCCAAAGCTGCAGACTTTAGGAGGTGTCCAAAGAATTAGAAAGAAAACA | | |
| | | GTTTTACAAAGATCAAAGGCCACTCAAGGTAAAGGTGGCTGCCCCCAAGAGAGATACAGG | | |
| | | AATTGTCAGGTCTTGAAGGTTTTGGTACTGTGCTTATATGTGGGATTGCTTTTACTCTCT | | |
| | | GTCAGAAGAGTCCAGGTTTTTCAAGGATATCAGCAAAACAATCTTGGTTTATTATTGTGA | | |
| | | TTCATATTATGCCTCAGGGACATTTCACTTGGATGATAACTAGTAATAAAAAACTAGACA | | |
| | | TGTCTAAAAAAAAAAAAA | | |
| | Identity | various ESTs which, however, do not completely cover the contig | | |
| | | >gb\|AA986577.1\|AA986577 uc81e12.y1 Sugano mouse kidney mkia Mus musculus cDNA | | |
| | | clone IMAGE:1432078 5′. | | |
| | | Length = 553 | | |
| | | Score = 1082 bits (546), Expect = 0.0 | | |
| | | Identities = 551/553 (99%) | | |
| | | Strand = Plus/Plus | | |
| | | >gb\|AA137597.1\|AA137597 mq28c01.r1 Barstead MPLRB1 Mus musculus cDNA | | |
| | | clone IMAGE:580032 5′. | | |
| | | Length = 556 | | |
| | | Score = 1074 bits (542), Expect = 0.0 | | |
| | | Identities 542/542 (100%) | | |
| | | Strand = Plus/Plus | | |
| | | >gb\|AA388338.1\|AA388338 vc94d05.r1 Barstead MPLRB1 Mus musculus cDNA | | |
| | | clone IMAGE:790665 5′. | | |
| | | Length = 503 | | |
| | | Score = 545 bits (275), Expect = e−152 | | |
| | | Identities = 282/283 (99%), Gaps 1/283 (0%) | | |
| | | Strand = Plus/Plus | | |
| | | >gb\|A1098009.1\|A\|098009 vg82f08.r1 Barstead MPLRB1 Mus musculus cDNA | | |
| | | clone IMAGE:872487 5′. | | |
| | | Length = 467 | | |
| | | Score = 910 bits (459), Expect = 0.0 | | |
| | | Identities = 465/467 (99%) | | |
| | | Strand = Plus/Plus | | |
| | | >gb\|AA986094.1\|AA986094 uc81f01.x1 Sugano mouse kidney mkia Mus musculus cDNA | | |
| | | clone IMAGE:1432057 3′. | | |
| | | Length = 555 | | |
| | | Score = 745 bits (376), Expect = 0.0 | | |
| | | Identities = 3761376 (100%) | | |
| | | Strand = Plus/Minus | | |
| | Properties | Prediction: 2TM-Dom. (C- + N-Term.intracetl.) 33% ER. 22% CM, 22% vacuole, 11% mito, 11% Golgi | | |
| Clone # | T7 sequence | AGCTCGGCGCCGCCTGAGCGCCCGGCCCGACCCCGCCATGGGGTGCTGCTATAGCAGCGAAAACGAGGAGTCGGAC | | |
| 2 | | CAGGATCGGGAGGAGAGGAAGCTGCTGCTGGACCCCAGTAGCACCCCTACCAAAGCCCTCAATGGAGCCGAGCCCA | | |
| | | ACTACCATAGCCTACCTTCAGCTCGCACAGATGAGCAGGCCCTGCTTTCCTCCATCCTTGCCAAGACAGCTAGCAAC | | |
| | | ATCATTGATGTGTCTGCCGCAGACTCCCAGGGCATGGAACAGCATGAGTACATGGACCGGGCAAGGCAGTACAGTA | | |
| | | CCCGCTTGGCTGTGCTTAGCAGCAGTCTGACCCATTGGAAGAAGCTGCCACCGTTGCCATCTCTCACCAGCCAGCCC | | |
| | | CACCAAGTGCTGGCCAGTGAGCCTATCCCCTTCTCTGACTTGCAGCAGGTCTCCAGGATAGCTGCGTATGCCTATAG | | |
| | | TGCACTTTCTCAGATCCGCGTGGATGCGAAAGAAGAGCTGGTTGTACAGTTTGGGATCCCATGAAGAGAGGGGCCC | | |
| | | TAGGACAGCTCTTCCCTCGTCTTCACCCCGTCTCCACCCCACCTCTTCTGGCCCCCAGCCTCACTGNGGCTCTCTACA | | |
| | | GTACCTAACCTGCTACTAATCACGGAGAAGAATGTGGAGGGAAAGAACAAGGCTGGAGGCCGGAGCAAGTGAGGAC | | |
| | | TAAGC | | |
| | BGH sequence | CAAATGAATATACTTTCTTTATCGAGGGGTGACAAACAAAAACAAAAAGAGCAAACATGTAAAAACCCAGGGTGCTA | | |
| | | GAAATACAAACTCAATTCAGACTCAAGCTCGTCTAGACCCTGGTCATAATCCCCAGTGAGGTGCCTGTGAGCACCAA | | |
| | | GTCAGGGAAGGGGACAGGAGTGAATCGGAGGCCAAGAGAAAGAGGGCAGGAAGGGATCTCCTAGGTCTCCCGGNG | | |
| | | TCACCCCTACAGNGGTATGTCCATCTTCCCAATGACTGAAGATCTGCCAGGCCCTGTCCTCTTGGCCCCAACCTNACC | | |
| | | CTAACCAGAGCATGAAGGCCGATGGCAATCGGTCCTCCCTTCCCTTGCTTAGTCCTCACTTGCTCCGGCCTCCAGCG | | |
| | | TTGTTCTTTCCCTCCACATTCTTCTCCGTGATTAGTAGCAGGTTAGGTACTGTAGAGAGCCACAGTGAGGCTGGGG | | |
| | | GCCAGAANAGGTGGGGTGGAGACGGGGTGAAGACGAGGGAAGAGCTGTCCTAGGGCCCCTCTCTTCATGGGATCC | | |
| | | CAAACTGTACAACCAGCTCTTCTTTCGCATCCACGCGGATCTGAGAAAGTGCACTATAGGCATACGCAGCTATCCTG | | |
| | | GAGACCTGCTGCA | | |
| | Contig | AGCTCGGCGCCGCCTGAGCGCCCGGCCCGACCCCGCCATGGGGTGCTGCTATAGCAGCGAAAACGAGGACTCGGAG | | |
| | | CAGGATCGGGAGGAGAGGAAGCTGCTGCTGGACCCCAGTAGCACCCCTACCAAAGCCCTCAATGGAGCCGAGCCCA | | |
| | | ACTACCATAGCCTACCTTCAGGTCGCACAGATGAGCAGGCCCTGCTTTCCTCCATCCTTGCCAAGACAGCTAGCAAC | | |
| | | ATCATTGATGTGTCTGCCGCAGACTCCCAGGGCATGGAACAGCATGAGTACATGGACCGGGCAAGGCAGTACAGTA | | |
| | | CCCGCTTGGCTGTGCTTAGCAGCAGTCTGACCCATTGGAAGAAGCTGCCACCGTTGCCATCTCTCACCAGCCAGCCC | | |
| | | CACCAAGTGCTGGCCAGTGAGCCTATCCCCTTCTCTGACTTGCAGCAGGTCTCCAGGATAGCTGCGTATGCCTATAG | | |
| | | TGCACTTTCTCAGATCCGCGTGGATGCGAAAGAAGAGCTGGTTGTACAGTTTGGGATCCCATGAAGAGAGGGGCCC | | |
| | | TAGGACAGCTCTTCCCTCGTCTTCACCCCGTCTCCACCCCACCTCTTCTGGCCCCCAGCCTCACTGTGGCTCTCTACA | | |
| | | GTACCTAACCTGCTACTAATCACGGAGAAGAATGTGGAGGGAAAGAACAAGGCTGGAGGCCGGAGCAAGTGAGGAC | | |
| | | TAAGCAAGGGAAGGGAGGACCGATTGCCATCGGGCTTCATGCTCTGGTTAGGGTNAGGTTGGGGCCAAGAGGACA | | |
| | | GGGCCTGGCAGATCTTCAGTCATTGGGAAGATGGAGATACCNCTGTAGGGGTGACNCCGGGAGACCTAGGAGATC | | |
| | | CCTTCCTGCCCTCTTTCTCTTGGCCTCCGATTCACTCCTGTCCCCTTCCCTGACTTGGTGCTCACAGGCACCTCACTG | | |
| | | GGGATTATGACCAGGGTCTAGACGAGCTTGAGTCTGAATTGAGTTTGTATTTCTAGCACCCTGGGTTTTTACATGT | | |
| | | TTGCTCTTTTTGTTTTTGTTTGTCACCCCTCGATAAAGAAAGTATATTCATTTG | | |
| | Identity | >dbj\|AK00632.1\|AK000632 Homo sapiens cDNA FLJ20625 fis, clone KAT04008 | | |
| | | Length = 1112 | | |
| | | Score = 813 bits (410), Expect = 0.0 | | |
| | | Identities = 596/650 (91%), Gaps = 6/650 (0%) | | |
| | | >gb\|W89554.1\|W89554 mf71g09.r1 Soares mouse embryo NbME13.5 14.5 | | |
| | | Mus musculus cDNA clone IMAGE:419776 5′. | | |
| | | Length = 471 | | |
| | | Score = 781 bits (394), Expect = 0.0 | | |
| | | Identities. = 399/401 (99%) | | |
| | | Strand = Plus/Plus | | |
| | | >gb\|AW536394.1\|AW536394 G0103H12-3 Mouse E7.5 Embryonic Portion cDNA Library | | |
| | | Mus musculus cDNA clone G0103H12 3′. | | |
| | | Length = 624 | | |
| | | Score = 1088 bits (549), Expect = 0.0 | | |
| | | Identities = 558/562 (99%) | | |
| | | Strand = Plus/Minus | | |
| | Properties | DNA ladder | | |
| Clone # | T7 sequence | AGTCTGGCTTCACGCTNCANNAGTGNCGAGCGCCTCACGGAGGAAGAGTTGCATATCATCGCGCAGGTGCCGCCTC | | |
| 3 | | CACCCGCCGCTGCCGCCGTTGGCTACGCTGGCTGCCGTGTGGCTGTGACCTTCTTCCTCTACTTCCTGGCTACCAAC | | |
| | | TACTACTGGATTCTGGTGGAGGGACTGTACTTACACAGCCTCATCTTCATGGCCrTTTTCTCAGAGAAGAAGTATCT | | |
| | | GTGGGGCTTCACCATCTTTGGCTGGGGTCTGCCGGCTGTCTTCGTGGCTGTGTGGGTCGGTGTCAGAGCAAGCTT | | |
| | | GGCCAACACTGGGTGCTGGGACCTGAGCTCTGGGCACAAGAAGTGGATCATCCAGGTGCCCATCCTGGCATCTGTT | | |
| | | GTGCTCAACTTCATCCTCTTTATCAACATCATGCGGGTGCTTGCCACTAAGCTTNGGGAGACAAATGNGGGCCGGNG | | |
| | | TGACACCAGGCANTAGTACCGGAAGCTGCTNACGTCACNTTGGTGCCNGCGCCACTCTTTTTGTCACTACAGCCGTC | | |
| | | TTNATGGCCTTNCCGTACACCCGAGGTCTTNAGGGACACTTTGNCANATCCAGATCACTATNAGANTGCTTCTTTAA | | |
| | | ACTTCCTTTTCANGGGATTTTTTTT | | |
| | BGH sequence | CCCTTCTTTTTTTTTTTTTTTTTTCTNTTTTCCTTTNGTNTTCCNGGNCCAGTCCTGAANAGACAGCCAGCCAAANTT | | |
| | | TGGNTTNTNGGNCCATNTGTCCATNTGCCCATGCCAGCAGTCCAGCCCNTTGANGCCCAGTCANATGACTGNTTCCC | | |
| | | ATTNTTCCTGCAANAANGGAGGNGGCCGGCGCAAACCCANAGGCCTNNTAATCCANACCCGAGCAGGAGCCATTAAGG | | |
| | | AAGCCGTCGTCCTTGGGAACTGNCANAGNAACTGGNANGGTTTCGTTNTAAANGGNTGGAGCNCCCGGNTTGGCGN | | |
| | | GGCCAGGCAGNTGGNAGNGGCCANTGGNGGNGGNAGGAAGNAGGCGGNAGNTAAGGGGAAGGNTGAGTCCTGNAC | | |
| | | GG | | |
| | Identity | emb\|X78936\|MMPHRPR M. musculus mRNA for parathyroid hormone/parathyroid | | |
| | | hormone related peptide receptor | | |
| | | Length = 1984 | | |
| | | Score = 878 bits (443), Expect = 0.0 | | |
| | | Identities = 557/591 (94%), Positives = 557/591 (94%), Gaps = 7/591 (1%) | | |
| | Properties | DNA ladder | | |
| Clone # | T7 sequence | AGGCGCTGCCTACCAGAGCGCAGCATGACGGCCATCGGCGCGCAGGCCCACAAGCTGTTGGGCCTTAAGAGGCCCC | | |
| 4 | | ACCGGTCTTTCTTTGAGTCCTTCATCCGGACACTCATCATCGTGTGCACTGCCCTGGCTGTGGTCCTTTCTTCAGTC | | |
| | | TCCATCTGCGATGGCCACTGGCTCCTAGTGGAGGATCATCTCTTTGGGCTGTGGTACTTCTGCACCATCGGCAACC | | |
| | | ACAGTGAACCACACTGTCTGAGAGACCTGAGCCAGGCCCATATGCCCGGGCTGGCTGTAGGCATGGGCCTAGCACG | | |
| | | CAGTGTGGCCGCCATGGCAGTGGTGGCTGCCATCTTCGGCTTGGAGATGCTCATTGTGTCCCAGGTGTGTGAAGA | | |
| | | TGTCCGCTCACGGCGCAAGTGGGCCATCGGTTCCTACCTCCTNCTGGTTGCCTTTATCCTCTCCTCTGGGGGCCTCC | | |
| | | TCACCTTCATCATCCTGCTCAAGAATCAGATCAACCTTCTGGGCTTCACCCTGATGTTCTGGTGTGAATTCACTGNC | | |
| | | TNCTTCCTCTTCTTNCTNAATGCCGCCAGCGGGCTTTACATNAAAAGCTTACTTAGCCCTTGGACCCTCAGCAGGGA | | |
| | | CCCTGGCTTNCANAAAGNGAGGTTATGATGGGACTTTTTTTATTNTAGGGAANCTNTTGNCTGGNGATTTGCNCCCC | | |
| | | CCCCCNCCCCAATGNNCCTTTGACACNTTCCCTCTTTATGGGTAATTTNAAGTTTTTT | | |
| | BGH sequence | ACTTTCAAATTGAGATTTTAATAGCATGACTAACCTATCCAGCTCACTGTGCCGTCGTACAGAGGCACCCTTCTGCC | | |
| | | TTTGCCCTGGAGCTCAGCTGAAGAGACTTCCAGGGCATTGCTAGAGCTAAGTGCCTAAGAGGCAGTGTCAAGGTCA | | |
| | | TTGGTTGTCCTATTCAATCTNAGCCAGAGGTCCTATATNAGAGAAGTCCCATCATAACCTCGCTTTCTGTAAGCCAG | | |
| | | GGTCCCTGCTGGAGGGTCCCAGGGCTGAGTGAGGCTGTTGATGTGAAGGCCGCTGGCGGCATTGAGGAAGAAGAG | | |
| | | GAAGGAGGCAGTGAATTNACACCAGAACATNAGGGTGAAGCCCAGGAGGTTGATCTGATTCTTGAGCAGGATGATN | | |
| | | AANGTNAGGAGGCCCCCAGAGGAGAGGATAAAGGCAACCAGGAGGAGGTAGGAACCGATGGCCCACTTGCGCCGT | | |
| | | GAGCGGACATCTTTACACACCTGGGACACAATGAGCATCTTCAAGCCGAAGATGGCAGCCACCACTGCCATGGCGG | | |
| | | CCACACTGCGTGCTAGGCCCATGCCTACAGCCAGCCCGGGCATATGGGCCTGGCTCAGGTCTCTCAGACAGTGTGG | | |
| | | TCACTG | | |
| | Identity | >emb\|AJ272046.1\|MMU272046 Mus musculus mRNA for calcium channel gamma 5 | | |
| | | subunit (CACNG5 gene) | | |
| | | Length = 636 | | |
| | | Score = 1094 bits (552), Expect = 0.0 | | |
| | | Identities = 605/623 (97%), Gaps = 2/623 (0%) | | |
| | | Strand = Plus/Plus | | |
| Clone # | T7 sequence | AGCAGACTCAGGAAGAAACCATGGTGCTCTCTGGGGAAGACAAAAGCAACATCAAGGCTGCCTGGGGGAAGATTGG | | |
| 5 | | TGGCCATGGTGCTGAATATGGAGCTGAAGCCCTGGAAAGGATGTTTGCTAGCTTCCCCACCACCAAGACCTACTTCC | | |
| | | CTCACTTTGATGTAAGCCACGGCTCTGCCCAGGTCAAGGGTCACGGCAAGAAGGTCGCCGATGCTCTGGCCAATGC | | |
| | | TGCAGGCCACCTCGATGACCTGCCCGGTGCCCTGTCTGCTCTGAGCGACCTGCATGCCCACAAGCTGCGTGTGGAT | | |
| | | CCCGTCAACTTCAAGCTCCTGAGCCACTGCCTGCTGGTGACCTTGGCTAGCCACCACCCTGCCGATTTCACCCCCGC | | |
| | | GGTGCATGCCTCTCTGGACAAATTCCTTGCCTCTGTGAGCACCGTGCTGACCTCCAAGTACCGTAAGCTGCCTTNTG | | |
| | | CGGGGCTTGCCTTCTGGCCATGCCCTTCTTCTCTCCTTGCACCTGTACCTCTTTGGTTTTGAATAAAGCCTGAGTAG | | |
| | | GAAGAAAAAAAAAAAAAAANAGGGCGGNCGTTNGAGCATGCATCTAGAGGGGCCCTATTNTATAGTGNNACCTAAAA | | |
| | | ATGCTTAGAGNTTTGNTGNTCAGCCCT | | |
| | Identity | >ref\|NM_008218.1\| Mus musculus hemoglobin alpha, adult chain 1 (Hba-a1), | | |
| | | mRNA | | |
| | | Length = 564 | | |
| | | Score = 969 bits (489), Expect = 0.0 | | |
| | | Identities = 529/540 (97%), Gaps = 2/540 (0%) | | |
| | | Strand = Plus/Plus | | |
| | | >gb\|L75940.1\|MUSALGL Mus musculus alpha-globin mRNA, complete cds | | |
| | | Length = 513 | | |
| | | Score = 910 bits (459), Expect = 0.0 | | |
| | | Identities = 486/494 (98%), Gaps = 1/494 (0%) | | |
| | | Strand = Plus/Plus | | |
| Clone # | T7 sequence | GGTACCGAGCTCGGATCCACTAGTAACGGCCGCCAGTGTGCTGGAAAGGGGCTTCGGACCCGGAAGTGGCGCCTT | | |
| 6 | | GGGCTCCCGGCGGCGCCGCGGGGATGGCGGGAGCCGGAGCTGGTGCAGGAGCTCGGGGCGGCGCGCCGGCCGGA | | |
| | | GTCGAGGCCCGCGCTCGGGACCCGCCACCCGCGCACCGCGCGCACCCTCGCCATCCTCGGCCCGCGGCTCAGCCGT | | |
| | | CGGCGCGCAGGATGGACGGCGGCCCGGGCGCCCCGGGCTCCGGGGACAACGCCCCGACCACCGAGGCGCTGTTCG | | |
| | | TGGCGCTGGGCGCGGGCGTGACGGCTCTCAGTCACCCGCTGCTCTACGTGAAGCTGCTGATCCAGGTGGGTCATG | | |
| | | AGCCGATGCCCCCCACCCTTGCGACCAATGTGCTGGGGAGGAAGGTCCTCTACCTGCCGAGCTTCTTCACCTATGC | | |
| | | CAAGTACATTGTGCAGGTGGATGGGAAGATAGGGCTCTTCCGGGGCCTGAGCCCCCGCCTTATGTCGAACGCCTTG | | |
| | | TCCACTGTGACCCGCGGCAGCATGAAGAAGGTTTTCCCTCCAGATGAGATGGAGCAGGTTTCCAACAAGGACGACA | | |
| | | TGAAGACCTCACTCAAGAAAGTTGTGAAGGAGACATCGTATGAGATGATGATGCAGTGTGTATCGCGAATGCTGGC | | |
| | | CCATCCCTTACACGTGATCTCGATGCGATGCATGGTGCAGTTTGTGGGACGGGAGGCCAAGTACAGTGTGTGCTGA | | |
| | | GTTCTATTGGGAGATCTTCAAGGAAGAGGGCTGCTGGGATTCTTCGTTGGCTTAATCCCTCA | | |
| | R2 sequence | GACTTGAACTCAGGGGAATTCGGaGGGAGTCCAGCCCGCAGCCCACAGTTGTTCACTGcCATGAGATCTCCAACGA | | |
| | | GCAGGAAGGGGTAGGTCAGCATGCTCACTGCAATCCCCATCACAAACTTGGTGTAGCTCCGGATGGCCAGGGCCTG | | |
| | | GCTAAAGCTGTCGTCCACCAAGTAGGCATTGATGAAGTGGGCCAGCAGGTTACAGCCCCACAAGAAAACCACATCGC | | |
| | | CCAGGAGGTGAGGGATTAAGCCAACGAAGAATCCCAGCAGCCCCTCTTCCTTGAAGATCTTCCCAATAGAACTCAGC | | |
| | | ACACCACTGTACTTGGCCTCCCGTCCCACAAACTGCACCATGCATCGCATCGAGATCACGTGTAAGGGATGGGCCAG | | |
| | | CATTCGCGATACACACTGCATCATCATCTCATACGATGTCTCCTTCACAACTTTCTTGAGTGAGGTCTTCATGTCGTC | | |
| | | CTTGTTTGGAAACCTGCTCCATCTCATCTGGAGGGAAAACCTTCTTCATGCTGCCGCGGGTCACAGTGGACAAGGC | | |
| | | GTTGGACATAAGGCGGGGGCTCAGGCCCCGGAAGAGCCCTATCTTCCCATCCACCTGCACAATGTACTTGGCATAg | | |
| | | GTtGtAGAAGCTCGGCAGGTA | | |
| | 4SP6 sequence | TAGATGCATGCTCGAGCGGCCGCCCTTTTTTTTTTTTTTTTTTCCAAATCACCACCAATACATTTATTCGAGGAGAT | | |
| | | GGGTCTATCTTACCACGAGGGGAGGACTAGATGTCGGCCTATGTAACCTGTGCGTATTCGCAGCCAGCAGAGTGAC | | |
| | | TGAACCCTCACACCTGGCGTCACCAGCACAGACAAGCAGATGAGGGGATGGTCTGAGGAGAACATGATTTCCTATT | | |
| | | CAGGAGAAGGCACCACCCTTGTATAAGAAAATTAGTGTTGGGAACATAGCGCCAGCCTCCCATGGCCCAGGTGTGA | | |
| | | TGGCGCCCAATTTACAAAGCAGGAAGTGGGGGGCGGGGGTGCTTCTGGCTGACTGGCAGGATGAGCTGGGCTAGA | | |
| | | GGTGCAGGGAAGCCTTGCCACTGAGTGACGTTTGCCTCTGCAGCCTGCCTCTGCCTGAGTACAAGATGGACTCCAG | | |
| | | TACCTCTAGGCAGGAAGGGGATGCCACCCCAACACTGCTCCCCCAGGCTTGCCCAGGTCCCAGGTGAGCCACCTCCA | | |
| | | CCAGCCCACATCTGGACAGACACATGGCAAATATGGACATGAAGCCAGGCTGGGCTGGAGTCACCGTTGGGCATCT | | |
| | | GAGATGATGGGTCCTCACCAGGGGTAGGCTAAGACCCCATAAATGTTTGGTCGGGCAGCTTAGGTTAGTCCAGGGC | | |
| | | AAAGCATGACCCCGATGACACCCGGCGGAAAAGCAGGCTGGAGCCGCGGAAGAGCTGGCCCTGCACACTCAGGTAC | | |
| | | TTCCAGCAGTGGATCCAGGACTTGAACACAGGGGAATACGGAGGAGTCCAGCCCGCAGCCCACAGTTGTTCACTGC | | |
| | | CATGAGATCTCCAACGAGCAGGAAGGGGTAGGTCAGCATGCTCACTGCAATCC | | |
| | Contig | GGTACCGAGCTCGGATCCACTAGTAACGGCCGCCAGTGTGCTGGAAAGGGGCTTCGGACCCGGA | | |
| | | AGTGGCGCCTTGGGCTCCCGGCGGCGCCGCGGGGATGGCGGGAGCCGGAGCTGGTGCAGGAGCT | | |
| | | CGGGGCGGCGCGCCGGCCGGAGTCGAGGCCCGCGCTCGGGACCCGCCACCCGCGCACCGCGCGC | | |
| | | ACCCTCGCCATCCTCGGCCCGCGGCTCAGCCGTCGGCGCGCAGGATGGACGGCGGCCCGGGCGC | | |
| | | CCCGGGCTCCGGGGACAACGCCCCGACCACCGAGGCGCTGTTCGTGGCGCTGGGCGGGGGCGTG | | |
| | | ACGGCTCTCAGTCACCCGCTGCTCTACGTGAAGCTGCTGATCCAGGTGGGTCATGAGCCGATGC | | |
| | | CCCCCACCCTTGGGACCAATGTGCTGGGGAGGAAGGTCCTCTACCTGCCGAGCTTCTTCACCTA | | |
| | | TGCCAAGTACATTGTGCAGGTGGATGGGAAGATAGGGCTCTTCCGGGGCCTGAGCCCCCGCCTT | | |
| | | ATGTCCAACGCCTTGTCCACTGTGACCCGCGGCAGCATGAAGAAGGTTTTCCCTCCAGATGAGA | | |
| | | TGGAGCAGGTTTCCAACAAGGACGACATGAAGACCTCACTCAAGAAAGTTGTGAAGGAGACATC | | |
| | | GTATGAGATGATGATGCAGTGTGTATCGCGAATGCTGGCCCATCCCTTACACGTGATCTCGATG | | |
| | | CGATGCATGGTGCAGTTTGTGGGACGGGAGGCCAAGTACAGTGGTGTGCTGAGTTCTATTGGGA | | |
| | | AGATCTTCAAGGAAGAGGGGCTGCTGGGATTCTTCGTTGGCTTAATCCCTCACCTCCTGGGCGA | | |
| | | TGTGGTTTTGTTGTGGGGCTGTAACCTGCTGGCCCACTTCATCAATGCCTACTTGGTGGACGAC | | |
| | | AGCTTTAGCCAGGCCCTGGCCATCCGGAGCTACACCAAGTTTGTGATGGGGATTGCAGTGAGCA | | |
| | | TGCTGACCTACCCCTTCCTGCTCGTTGGAGATCTCATGGCAGTGAACAACTGTGGGCTGCGGGC | | |
| | | TGGACTCCCTCCGTATTCCCCTGTGTTCAAGTCCTGGATCCACTGCTGGAAGTACCTGAGTGTG | | |
| | | CAGGGCCAGCTCTTCCGCGGCTCCAGCCTGCTTTTCCGCCGGGTGTCATCGGGGTCATGCTTTG | | |
| | | CCCTGGAGTAACCTAAGCTGCCCGACCAAACATTTATGGGGTCTTAGCCTACCCCTGGTGAGGA | | |
| | | CCCATCATCTCAGATGCCCAAGGGTGACTCCAGCCCAGCCTGGCTTCATGTCCATATTTGCCAT | | |
| | | GTGTCTGTCCAGATGTGGGCTGGTGGAGGTGGGTCACCTGGGACCTGGGGAAGCCTGGGGGAGC | | |
| | | AGTGTTGGGGTGGCATCCCCTTCCTGCCTAGAGGTACTGGAGTCCATCTTGTACTGAGGCAGAG | | |
| | | GCAGGCTGCAGAGGCAAACGTCACTCAGTGGCAAGGCTTCCCTGCACCTCTAGGCCAGCTCATC | | |
| | | CTGCCAGTCAGCCAGAAGCACCCCCGCCCCCCACTTCCTGCTTTGTAAATTGGGCGCCATCACA | | |
| | | CCTGGGCCATGGGAGGCTGGCGCTATGTTCCCAACACTAATTTTCTTATACAAGGGTGGTGCCT | | |
| | | TCTCCTGAATAGGAAATCATGTTCTCCTCAGACCATCCCCTCATCTGCTTGTCTGTGCTGGTGA | | |
| | | CGCCAGGTGTGAGGGTTCAGTCACTGTGCTGGGTGCGAATACGCACAGGTTACATAGGCCGACA | | |
| | | TCTAGTCCTCCCCTCGTGGTAAGATAGACCCATCTCCTCGAATAAATGTATTGGTGGTGATTTG | | |
| | | GAAAAAAAAAAAAAAAAAAGGGCGGCCGCTCGAGCATGCATCTA | | |
| | Identity | >gb\|AF176007.2\|AF176007 Mus musculus domesticus mitochondrial carrier | | |
| | | homolog 1 isoform a mRNA, complete cds; nuclear gene for mitochondrial product | | |
| | | Length = 1877 | | |
| | | Score = 3382 bits (1706), Expect = 0.0 | | |
| | | Identities = 1733/1746 (99%) | | |
| | | Strand = Plus/Plus | | |
| | | >gb\|AF151822.1\|AF151822 Homo sapiens CGI-64 protein mRNA, | | |
| | | complete cds | | |
| | | Length = 1734 | | |
| | | Score = 1166 bits (588), Expect = 0.0 | | |
| | | Identities = 879/972 (90%), Gaps = 3/972 (0%) | | |
| | | Strand Plus/Plus | | |
| | | >gb\|AF189289.1\|AF189289 Homo sapiens presenilin-associated protein mRNA, complete cds | | |
| | | Length = 1918 | | |
| | | Score = 948 bits (478), Expect = 0.0 | | |
| | | Identities = 727/810 (89%) | | |
| | | Strand = Plus/Plus | | |
| Clone # | T7 sequence | AGCTCCGCCCCTGCTACTGGACCATGGAGACTGTGGCCCAGTAGAGACCTTAGTGTGAGGCTTTCAGGGGCGGCG | | |
| 7 | | GCCATGGAGGCCGTGCTGAACGAGCTGGTGTCTGTGGAGGATCTGAAGAATTTTGAAAGGAAATTTCAGTCTGAGC | | |
| | | AGGCAGCTGGTTCTGTGTCCAAGAGCACGCAATTTGAATATGCCTGGTGCCTGGTTCGAAGCAAATACAATGAGGA | | |
| | | CATCCGCAGAGGCATCGTGCTGCTGGAGGAGCTGTTGCCCAAAGGGAGCAAAGAGGAACAGCGGGACTATGTCTTC | | |
| | | TACCTGGCCGTGGGCAACTACCGGCTCAAGGAATATGAAAAGGCTCTAAAGTATGTGCGAGGGCTGTTGCAGACTG | | |
| | | AGCCCCAGAACAACCAGGCCAAGGAGCTGGAACGCCTGATTGATAAGGCCATGAAGAAAGATGGACTGGTAGGCAT | | |
| | | GGCCATCGNTGGTGGCATGGCCCTGGGCGTGGCAGGCCTGGCTGGACTCATTGGACTGGCTGTCTTCCAAGTNCA | | |
| | | AATCCTGAAGGCAGNCTNACCTGCTCTNTTGCCCGGGACGCCTAGGAGCCTGGGGGACACTGGAAGAGGGGCCTGT | | |
| | | CCATACTACCATCGCCTTCCTTTTTTCTGCACCCCTGTAGTCTACCTTTACAGCTTCATGACCCCCAGCCTTTTAANN | | |
| | | CCTNCACCTGGTNGTTTAACCCTNTCATTCCTTTGCAATGAGTGNNAAATAAAAATTGGCCCC | | |
| | BGH sequence | CCCGAGCCACGGCCCAATTTTATTTACACTCATTGCAAAGNATGACAGGGTTAAACGACAGGTGCAGGGGCTAANAG | | |
| | | GCTGGGGGTCATGGAGACTGTAGAGGTAGACTACAGGGGTGCAGGANAAAAGGGAAGGCGATGGTGAGGATGGA | | |
| | | CAGGCCCCTNTTCCAGNGTCCCCCAGGCTCCTAGGCGTCCCGGGGCAGAGAGCAGGTGAGGCTGCCTTCAGGATTT | | |
| | | GGACTTGGAGACAGCCAGTCCAATGAGTCCAGCCAGGCCTGCCACGCCCAGGGCCATGCCACCAACGATGGCCATG | | |
| | | CCTACCAGTCCATCTTTCTTCATGGCCTTATCAATCAGGCGTTCCAGCTCCTTGGCCTGGTTGTTCTGGGGCTNAGT | | |
| | | NTGNAACAGCGCTCGCACATACTTTAGAGCCTTTTCATATTCCTTGAGGCGGNAGTTGCCCACGGCCAGGTAGAAGA | | |
| | | CATAGTCCCGCTGTTCCTCTTTGCTCCCTTTGGGCAACAGNTCCTCCAGCAGCACGATGCCTCTGCGGATGTCCTCA | | |
| | | TTGAATTTGCTTCGAACCAGGCACCAGGCATATTCAAATTGCGTGCTCTTGGACACAGAACCAGNTGCCTGCTNAGA | | |
| | | CTGAAATTTCCTTTCAAAATTNTTCAGATCCTTCACAGACACCAGCTCGTTCAGCACGGNCTCCATGGCCGCCGCCCN | | |
| | | TGAAAGCCTTACACTAAGGNCTNTACTGGGCCACAAGTTTCCATGGTCCAGAGCANGGGCCGGAGCTTNNAN | | |
| | Identity | >gb\|AF151893.1\|AF151893 Homo sapiens CGI-135 protein mRNA, complete cds | | |
| | | Length = 735 | | |
| | | Score = 434 bits (219), Expect = e−119 | | |
| | | Identities = 402/466 (86%) | | |
| | | Strand = Plus/Minus | | |
| | Properties | PI-FACS: Apoptosis | | |
| Clone # | T7 sequence | AGATTGACTTGGGCACTGACATGGTTCCTGCCATCTCCCTGGCCTACGAGCAAGCTGAGAGCGACATCATGAAGAG | | |
| 8 | | GCAGCCCAGAAACCCCAAAACGGACAAACTTGTGAACGAGCGTCTGATCAGCATGGCCTATGGACAGATCGGTATG | | |
| | | ATCCAGGCCCTGGGAGGCTTCTTCACTTACTTTGTGATTCTGGCTGAGAACGGTTTCCTGCCCTTTCACCTGTTGG | | |
| | | GCATCCGAGAGACCTGGGATGACCGCTGGGTCAACGATGTGGAGGACAGCTACGGGCAGCAGTGGACCTACGAGC | | |
| | | AGAGGAAGATCGTGGAGTTCACCTGCCATACAGCGTTCTTTGTCAGTATTGTGGTAGTGCAGTGGGCCGACTTGGT | | |
| | | CATCTGCAAGACCAGAAGGAATTCTGTGTTCCAGCAGGGAATGAAGAACAAGATCTTGATATTTGGCCTCTTTGAAG | | |
| | | AGACAGCCCTTGCTGCTTTCTTATCCTACTGCCCCGGGATGGGGGCAGNCCTTAGGATGTATCCCTCAAACCTACAT | | |
| | | GGTGGGTCTGTGCCTTTCCCTACTTCCCTTCTTAATCTTTTGTGTATGACGAGGGTGCGGGAAGCTTAATTATTAA | | |
| | | GGCGGGNGCCCCTTGGCNGGNTTGGGTTGGGAGGAANGGAGGACCCTACTTACTTAGGCCCCACTTGCCCCTTGG | | |
| | | NACGCCCGGGGGGGAAACAATTTTGTGNCCAACAACACCTTGNAACCCCAACCCCCTTACCCCCCCTTTTTTTGGGG | | |
| | | GGACCTTTCAAAGGGTTTTTGGGAGGCCTTNGGGAAACTTTTTACCCCTTGGGNNGGGGAAAAGGCACCCCAAAAC | | |
| | | CNTTTGTTGGGGGGATTGCCAAAACCNTTCCTTGGAAATGGAAAANAATTGTTANCTTNTAACC | | |
| | BGH sequence | CCGTTATAATAGCCATCTTTATTTGTAAAAATCCAGATATAAAACGTAATCTTTCAGTCTTTCCAGGTTTTCCTTTTT | | |
| | | TACAAAAACAAAAAGGCACGTATAAACCTTGCCCGCTGTCGTCCCCGTACACGGNGTTTCTCAGGCAGCCCTCCCCC | | |
| | | CCGCCCCGCCCCCCGTTACAGCTACATGCTTCATTCCAGGACGTCTGCATCCCCACATGCTTNGGNGCTTTCCTACC | | |
| | | AGGGTAGAGTTCCGAGCTCCAAGACTTGAAGTACACAAAGAGGGGGTAGGGGNGGGNGCAGNGNGTGGCACAATG | | |
| | | TTCCACGGCGTGCAGGGCAGAGGGCTAGTAGTAGGTCTCCTTCTCCACCCAGCCGCAGGGCGCCGCCTGATAATGA | | |
| | | GCTTCCGCACCTNGNATACACAAAGATGAGAAGGGAGTAGGGGAAGGCACAGAACCACCATGTAGGTTTGAGGGGA | | |
| | | TACATNCTAAGGG | | |
| | Identity | >ref\|NM_012504.1\| Rattus norvegicus ATPase, Na + K + transporting, | | |
| | | alpha 1 polypeptide (Atp1a1), mRNA | | |
| | | Length = 3636 | | |
| | | Score = 874 bits (441), Expect = 0.0 | | |
| | | Identities = 528/556 (94%), Gaps = 1/556 (0%) | | |
| | | Strand = Plus/Plus | | |
| Clone # | T7 sequence | AGCCGGAGGCCGAGCCCAGTCGCCAGCTCCTGCTCTGCTCCTCTCCCGCCTGCCGCCGCGCTGCACGCCTCGAGCA | | |
| 9 | | CTCCCTCGGCCCCGGCGGGGACCGGGGACCCCGCAGCTACCGCCATGCTGCCAGTGCTCTACACCGGCCTGGCGGG | | |
| | | GCTGCTGCTGCTGCCTCTGCTGCTCACCTGCTGCTGCCCCTACCTCCTCCAAGATGTGCGGTACTTCCTGCGGCTG | | |
| | | GCCAACATGGCCCGGCGGGTGCGCAGCTACCGGCAGCGGCGACCCGTGCGTACCATCCTGCGGGCCTTCCTGGAAC | | |
| | | AAGCGCGCAAGACCCCACACAAGCCCTTCCTGCTGTTCCGAGACGAGACGCTCACCTACGCCCAGGTGGACCGGCG | | |
| | | CAGCAACCAAGTGGCGCGGGCGCTGCACGATCAACTGGGCCTACGACAGGGGGATNGCGTAGCCCTCTTCATGGGC | | |
| | | AATGAGCCGGCCTACGTGTGGATCTGGCTGGGACTGCTCAAACTGGGCTGTCCCATGGCGTGCCTCAACTACAACA | | |
| | | TTCGTGCCAAGTCTCTGCTGCACTGCTTTCAATGCTGCGGGGCGAAGGTGCTGCTGGCCTNCCCAGATCTACAAGA | | |
| | | AGCTGTGGAGGAG | | |
| | BGH sequence | AGTGTTAATATAGTTTATTATGTCTTTAAAAAAATAAGGCCCTCTCTCCAAGAAGCTTAGTTTGCAAGGACAAATGG | | |
| | | CAGGTGCACATTGAAAAATAATTGTTTCTAAATCTTTTTACTTGCAAAGGTTCAGGTGTAATTTAAAAAAAAAACAAA | | |
| | | CAAACTATCCTTTTAATGAATAATTTCCTAAAAATAAAATCGCACCTTATAGCCTTNAATCAAGTTAAAGTTGGATTC | | |
| | | TACGTATGAAGTGGCTCTGCGAGGTCTATCGAGTTTCTTTCTGGAAATGTCATGAGCTAAACCACCAGGGAATATTC | | |
| | | AGAGCTTNAGAGTTTTATCAATTATGGCATTATAAATGTTCTCAGTCATGGGCACAAATGTTTTCTCTGCATCATCC | | |
| | | ATGAAATACAAGGTATCTTTGATGACTGTGGGATTGAAGCCCTCTTCCATCAGGGTCACTTTGCGGNGTTTAAAAGT | | |
| | | CCCAGTGATCTCAATGGTATCTTGTATCCTCAGGAACCGAGGCCTCGCGTAACTGGGCAGGTACTCCGCGATGTGT | | |
| | | TGAAAGAGTTTCTTTCCATTGAACTCGTAGT | | |
| | Identity | >ref\|NM_011978.1\| Mus musculus solute carrier family 27 (fatty acid | | |
| | | transporter), member 2 (Slc27a2), mRNA | | |
| | | Length = 1872 | | |
| | | Score = 844 bits (426), Expect = 0.0 | | |
| | | Identities = 430/432 (99%) | | |
| | | Strand = Plus/Plus | | |
| | | >gb\|AF072757.1\|AF072757 Mus musculus fatty acid transport protein 2 mRNA, | | |
| | | complete cds | | |
| | | Length = 1872 | | |
| | | Score = 844 bits (426), Expect = 0.0 | | |
| | | Identities = 4301432 (99%) | | |
| | | Strand = Plus/Plus | | |
| | | >emb\|AJ223958.1\|MMAJ3958 Mus musculus mRNA for very-long-chain acyl-CoA | | |
| | | synthetase (VLACS) | | |
| | | Length = 2238 | | |
| | | Score = 844 bits (426), Expect = 0.0 | | |
| | | Identities = 430/432 (99%) | | |
| | | Strand = Plus/Plus | | |
| Clone # | T7 sequence | AGCGCGGGAGGCGCATGGCGGGCATGGCGCTGGCGCGAGCATGGAAGCAGATGTCCTGGTTCTACTACCAGTACC | | |
| 10 | | TGCTGGTCACTGCGCTCTACATGCTGGAGCCCTGGGAGCGAACCGTGTTCAATTCGATGCTGGTTTCCGNGGTGGG | | |
| | | GATGGCCCTGTACACTGGCTACGTCTTCATGCCCCAGCACATCATGGCTATTCTGCATTACTTTGAAATTGTACAGT | | |
| | | GACGAAGATGTGACCAGGATCCAGAGGTTGCTGGGGAAGATCTGCCTTGTGAAGTTGGAATGAGACCTCATCAGAT | | |
| | | GTAAGATGTGCTACGGATGTCCACGTGACCAACCTTATAAATACAAAGACTTTAAAAAAAACTTNATGAGTAGAACA | | |
| | | GGAAAAATCATCCTGGCTCATGTGTTGNGTTCTTTCTTTTTGATTTTAACAGAGGCTCTTATATAGTAGCTTTTATC | | |
| | | TATTTTAACATTGTAGTCATTTGTACTTTGATATCAGTATTTTCTTAACCTTTGTGACTGTTTCAATATTATCCAGTG | | |
| | | AAAGCTTTTCTTAATGTAACTTTGAGTACATCTCAATTGCCTTCTATTTTTAAAACCTAAGGTCATTAGTTGGGCTTT | | |
| | | ACTGGTCTTGCTATCATATGGCATATACATCTGCCTGGATATATTTCTACTCTTGACCA | | |
| | BGH sequence | ACATTTTTCTTTGAATTTAATGAGTTTACATNAAAAAAAAGTAGTCATTTTACATNTAAGGAATAAAAACCGTTTTAA | | |
| | | AAAAAATACAAAGAGTGAAAGGATTTTTAAGCAAGTTTACATTTCTTTTGGTTATGGTTCTGCACAATTCATCTCATT | | |
| | | GNGTCTTTATNACAACGTGCAAATGCATTTNACAACGCCTGTTACAACATNAAATTAACTNTTGAGCGTATACAGGG | | |
| | | TCAATACTGCCTNAGAGGATCTGATAAGCCTTCTATGAAAAGCTNCACAGTGTATNTNAGCATATGTCATACAAGCC | | |
| | | GGCCACCAATCACCAATNACAGGAATCATNAAAGTTGGTTGGAAATAAGTCCACATAAGAATTTAATATNTAAAAGG | | |
| | | NGAAATGTTCCTTGTATTAATGTTAGCAAGATCTTTACTTTTTCATTACTAAGAAACACTTTAATAGTTTTAGAGCAA | | |
| | | AAGCTGTTAAGAGTCTAGGGAGCTAAAACCGTACTCCTGAGTTCAAGCAAGCAGATAAATCTTTTGTAAGTAGTTCT | | |
| | | NAAAGTATCCTCCCTCCCGTCCCCAAATTCTGTATTGNTTCTTACAAAACTTTGGTCAAGAGTNGAAATATATCCAGG | | |
| | | CAGATGTATATGCCATATGATAGCAAGAACAGTA | | |
| | Contig | AGCGCGGGAGGCGCATGGCGGGCATGGCGCTGGCGCGAGCATGGAAGCAGATGTCCTGGTTCTACTACCAGTACC | | |
| | | TGCTGGTCACTGCGCTCTACATGCTGGAGCCCTGGGAGCGAACCGTGTTCAATTCGATGCTGGTTTCCGTGGTGGG | | |
| | | GATGGCCCTGTACACTGGCTACGTCTTCATGCCCCAGCACATCATGGCTATTCTGCATTACTTTGAAATTGTACAGT | | |
| | | GACGAAGATGTGACCAGGATCCAGAGGTTCCTGGGGAAGATCTGCCTTGTGAAGTTGGAATGAGACCTCATCAGAT | | |
| | | GTAAGATGTGCTACGGATGTCCACGTGACCAACCTTATAAATACAAAGACTTTAAAAAAAACTTCATGAGTAGAACA | | |
| | | GGAAAAATCATCCTGGCTCATGTGTTGWGTTCTTTCTTTTTGATTTTAACAGAGGCTCTTATATAGTAGCTTTTATC | | |
| | | TATTTTAACATTGTAGTCATTTGTACTTTGATATCAGTATTTTCTTAACCTTTGTGACTGTTTCAATATTATCCAGTG | | |
| | | AAAGCTTTTCTTAATGTAACTTTGAGTACATCTCAATTGCCTTCTATTTTTAAAACCTAAGGTCATTAGTTGGGCTTT | | |
| | | ACTGKTCTTGCTATCATATGGCATATACATCTGCCTGGATATATTTCTACTCTTGACCAAAGTTTTGTAAGAAAACAA | | |
| | | TACAGAA | | |
| | | TTTGGGGACGGGAGGGAGGATACTTTGAGAACTACTTACAAAAGATTTATCTGCTTGCTTGAACTCAGGAGTACGG | | |
| | | TTTTAGCTCCCTAGACTCTTAACAGCTTTTGCTCTAAAACTATTAAAGTGTTTCTTAGTAATGAAAAAGTAAAGATCT | | |
| | | TGCTAACATTAATACAAGGAACATTTCACCTTTTAGATATTAAATTCTTATGTGGACTTATTTCCAACCAACTTTNAT | | |
| | | GATTCCTGTGATTGGTGATTGGTGGCCGGCTTGTATGACATATGCTRAGATACACTGTGGAGCTTTTCATAGAAGG | | |
| | | GTTATCAGATCCTCTRAGGCAGTATTGACCCTGTATACGCTCAARAGTTAATTTRATGTTGTAACAGGCGTTGTRAA | | |
| | | ATGCATTTGCACGTTGTGATAAAGACNCAATGAGATGAATTGTGCAGAACCATAACCAAAAGAAATGTAAACTTGCT | | |
| | | TAAAAATCCTTTCACTCTTTGTATTTTTTTTAAAACGGTTTTTATTCCTTARATGTAAAATGACTACTTTTTTTTRAT | | |
| | | GTAAACTCATTAAATTCAAAGAAAAATGTAAAAAAAAAAANGCN | | |
| | Identity | >emb\|AL080066.1\|HSM800567 Homo sapiens mRNA; cDNA DKFZp564J142 (from clone | | |
| | | DKFZp564J1A2) | | |
| | | Length = 1308 | | |
| | | Score = 444 bits (223), Expect = e−122 | | |
| | | Identities = 296/319 (92%). Gaps = 2/319 (0%) | | |
| | | Strand Plus/Plus | | |
| | | Score = 315 bits (158), Expect = 3e−83 | | |
| | | Identities = 262/294 (89%), Gaps = 2/294 (0%) | | |
| | | Strand Plus/Plus | | |
| | | Score = 213 bits (107), Expect = 1e−52 | | |
| | | Identities = 177/197 (89%), Gaps = 5/197 (2%) | | |
| | | Strand Plus/Plus | | |
| | | Score = 62.3 bits (31), Expect = 5e−07 | | |
| | | Identities = 41/43 (95%), Gaps = 1/43 (2%) | | |
| | | Strand Plus/Plus | | |
| | | >dbj\|AK001993.1\|AK001993 Homo sapiens cDNA FLJ11131 fis, clone PLACE1006325, | | |
| | | highly similar to Homo sapiens mRNA; cDNA DKFZp564J142 | | |
| | | Length = 1274 | | |
| | | Score = 444 bits (223), Expect = e−122 | | |
| | | Identities = 296/319 (92%), Gaps = 2/319 (0%) | | |
| | | Strand = Plus/Plus | | |
| | | Score = 331 bits (166), Expect = 5e−88 | | |
| | | Identities = 264/294 (89%), Gaps = 2/294 (0%) | | |
| | | Strand Plus/Plus | | |
| | | Score = 213 bits (107), Expect = 1e−52 | | |
| | | Identities = 177/197 (890/a), Gaps = 5/197 (2%) | | |
| | | Strand = Plus/Plus | | |
| | | Score = 62.3 bits (31), Expect = 5e−07 | | |
| | | Identities = 41/43 (95%), Gaps = 1/43 (2%) | | |
| | | Strand = Plus/Plus | | |
| | | Score = 58.3 bits (29), Expect = 7e−06 | | |
| | | Identities = 30/31 (96%) | | |
| | | Strand Plus/Plus | | |
| | Description | DNA ladder | | |
| Clone # | T7 sequence | AGCGAGTTTGCAGACTTCTTGTGCGCAGCTAGCCGCCTNAGGTGTTNGAACCATGAATCTTTTACTCCTTTNGGCTG | | |
| 11 | | TCCTCTGCTTGGGAACAGCCTTAGCTACTCCAAAATTTGATNAAACCTTTAGTGCANAGTGGCACCAGTGGAAGTCN | | |
| | | ACGCACAGAAGACTGTATGGCACGAATGAGGAANAGTGGAGGANAGCGATATGGGAGAAGAACATGAGAATGATCC | | |
| | | AGCTACACAACGGGGAATACAGCAACGGGCAGNACGGCTTTTCCATGGAGATGAACGCCTTTGGTGACATGACCAA | | |
| | | TGAGGAATTCAGGCAGGNGGNGAATGGCTATCGCCACCAGAAGCACAAGAAGGGGAGGCTTTTTCAGGAACCGCTG | | |
| | | ATGCTTAAGATCCCCAAGTCTGNGGACTGGAGAGAAAAGGGTTGNGTGACTCCTGTGAAGAACCAGGGCCAGNGCG | | |
| | | GGTCTNGNNGGGCGTTTAGCGCATCGGGTTGCCTAGAAGGACAGATGTTCCTTAAGACCGGCAAACTGATCTNACT | | |
| | | GAGTNAACAGAACCTTGTGGACTGTTCTCACGCTCAAGGCAATCAGGGCTGTAACGGAGGCCTGATGGATTTTGCT | | |
| | | TTCCAGTACATTAAGGAAAATGGAGGTCTGNACTCGGAGGAGTCTTACCCCTATGAAGCAAAGGACNGGATCTTGT | | |
| | | NAATACAGAGNCGAGTTCGCTGTGGCTAATGACACAGGGTTCCGTGGATNTNCCTTAGCCAGGAGAAAGCCTCATG | | |
| | | AAGCTGTGGCTN | | |
| | BGH sequence | TTACACACACACTGAGCTAAAATTTATTTTTAAGAGGTAAAGAAAGTTTTATACAACCTTTATATAAAANAAATATTN | | |
| | | NAGACTNAGAATTAAGCACTAAGTTTTCAATATTATAAAGNTGTTTATAANAGGAGTTTTAAGTAGNGGTAACATTT | | |
| | | AACCCATGTAAAAATGGCAACAGAATTNAAATNATGANTTGGATCCTNAATGATTNAAGNACCANGGTTNGANTNAAT | | |
| | | AAGGGTTNGGTCAGTTTTAAGCTGAATTCGTTNGGACATAGAGNCCATAAGTCCTCATTANCGCTACCNATNAATTN | | |
| | | ANGACAGGATAGNTGGNCGCGGNGGCAAGTCCACAGAGGTNGNCCCGGTCTTNGGCTATTTNGATGTAGCCTTCCA | | |
| | | TACCCCATTCACTTCCCCAGCTGNTCTTGACAAGCCAATATTTATTCTTATTTGAATCTGNTCCTTNATAGCCATAGC | | |
| | | CNANCAACAGAACCCCATGGTCGAGGATCTTGCTGCTACAGTNGGGTTNATAGTAGATGCCTGAACTATAGAACTGG | | |
| | | AGAGACGGATGGCTTGCGTCCATAGCAACAGAAATAGGCCCCACAGTCGCACAGCCTTCATGAGGGCTTTCTGTTG | | |
| | | CTGAGGGATATCNACGAACCCTGTGTCATTAGCCACAGGGAACTNGGCTCTGTATTTACNAGAANCGGCCTTTGCTT | | |
| | | CANAGGGNGTAAGGACTTCTCCGNGTCCN | | |
| | Identity | >ref\|NM_009984.1\| Mus musculus cathepsin L (CtsI), mRNA | | |
| | | Length = 1374 | | |
| | | Score = 1277 bits (644), Expect = 0.0 | | |
| | | Identities = 701/726 (96%), Gaps = 1/726 (0%) | | |
| | | Strand Plus/Plus | | |
| | | >emb\|X06086.1\|MMMEPR Mouse mRNA for major excreted protein (MEP) | | |
| | | Length = 1374 | | |
| | | Score = 1277 bits (644), Expect = 0.0 | | |
| | | Identities = 701/726 (96%), Gaps = 1/726 (0%) | | |
| | | Strand = Plus/Plus | | |
| | | >gb\|J02583.1\|MUSCPR Mouse cysteine proteinase mRNA, complete cds | | |
| | | Length = 1276 | | |
| | | Score = 1277 bits (644), Expect = 0.0 | | |
| | | Identities = 702/726 (96%), Gaps = 2/726 (0%) | | |
| | | Strand = Plus/Plus | | |
| | Description | DNA ladder | | |
| Clone # | T7 sequence | AGATGGCTTCCAGCGGAGTGACTGNGAGCGCCGNCGGGTCGGCCAGCGAGGCCTNAGAGGTTCCAGACAACGTGG | | |
| 12 | | GAGACTGGCTCCGCGGCGTCTTCCGCTTCGCCACCGATCGAAACGACTTCCGGAGGAACTTGATCCTTAATTTGGG | | |
| | | ACTCTTTGCTGCGGGAGTCTGGCTGGCCAGGAACTTGAGTGACATTGATTTGATGGCCCCTNAGCCAGGGGTGTAG | | |
| | | CCAGAGAATGGAACTCCTGTGTATTCAGACTTTCCAAAGACAGCCTACTGTCTGNGACCACAAGATCCTACCTGAGT | | |
| | | GGCAGCTGAAGTTGACTCCCTCTCCTTGCCTGAACCC | | |
| | BGH sequence | ATGNGCAGGCTTTATTTGAAATCTTTTTCAAGAACCATTATTACTCTTNAGGACAAGGGCAAGGACCATCTTCTGCA | | |
| | | GAAAGTNGGGAACTGCACACAGAACCGTGCAGAGGCAACATNTTAGCCGACACTGGGGGANGGGGGGGCAGAGGG | | |
| | | GGGG | | |
| | Contig | TGRKATGGCTTCCAGCGGAGTGACKGTGAGCGCCGCCGGGTCGGCCAGCG | | |
| | | AGGCCTCAGAGGTTCCAGACAACGTGGGAGACTGGCTCCGCGGYGTCTTC | | |
| | | CGCTTCGCCACCGATCGAAACGACTTCCGGAGGAACTTGATCCTYAATTT | | |
| | | GGGACTCTTTGCTGCGGGAGTCTGGCTGGCCAGGAACTTGAGTGACATTG | | |
| | | ATTTGATGGCCCCTCAGCCAGGGGTGTAGCCAGAGAATGGAACTCCTGTG | | |
| | | TATTCAGACTTTCCAAAGACAGCCTACTGTCTGTGACCACAAGATCCTAC | | |
| | | CTGAGTGGCAGCTGAAGTTGACTCCCTCTCCTTGCCTGAACCCCCCCYCA | | |
| | | CTSTCTCCCCCATCCCCCAGTGTCGGCTRAGATGTTGCCTCTGCACGGTT | | |
| | | CTGTGTGCAGTTCCCAACTTTCTGCAGAAGATGGTCCTTGCCCTTGTCCT | | |
| | | GAAGAGTARTAATGGTTCTTGAAAAAGATTTCAAATAAAGCCTGCACATA | | |
| | | AAAAAAAAAGAGA | | |
| | Identity | >emb\|AL137721.1\|HSM802233 Homo sapiens mRNA; cDNA DKFZp761H221 | | |
| | | (from clone DKFZp761H221) | | |
| | | Length = 3316 | | |
| | | Score = 199 bits (99), Expect = 1e−48 | | |
| | | Identities = 148/165 (89%) | | |
| | | Strand = Plus/Plus | | |
| | | >gb\|L34839.1\|HUMTUM Homo sapiens over-expressed breast tumor protein mRNA | | |
| | | Length = 574 | | |
| | | Score = 199 bits (99), Expect = 1e−48 | | |
| | | Identities 148/165 (69%) | | |
| | | Strand = Plus/Plus | | |
| | | >gb\|AF216754.1\|AF216754 Homo sapiens over-expressed breast tumor protein | | |
| | | (OBTP) mRNA, complete cds | | |
| | | Length = 354 | | |
| | | Score = 195 bits (97), Expect = 2e−47 | | |
| | | Identities = 146/163 (89%) | | |
| | | Strand = Plus/Plus | | |
| | | gb\|U16813\|HSU16813 Human Bak-3 pseudogene, complete cds. | | |
| | | Length = 5408 | | |
| | | Score = 69.9 bits (35), Expect = 3e−10 | | |
| | | Identities = 56163 (88%) | | |
| | | Strand = Plus/Plus | | |
| | | completely identical with the following ESTs: | | |
| | | dbj\|C88895\|C88895 Mus musculus early blastocyst cDNA, clone 01B00051IK19 | | |
| | | Length = 470 | | |
| | | Score = 613 bits (309), Expect = e−174 | | |
| | | Identities = 330/339 (97%) | | |
| | | Strand = Plus/Plus | | |
| | | gb/AA268719\|AA268719 va99h10.r1 Soares mouse NML Mus musculus cDNA clone | | |
| | | 747619 5′ | | |
| | | Length = 439 | | |
| | | Score = 599 bits (302), Expect = e−169 | | |
| | | Identities = 328/337 (97%), Gaps = 1/337 (0%) | | |
| | | Strand = Plus/Plus | | |
| Clone # | T7 sequence | TACCGAGCCCGGATCCCTAGTAACGGCCGCCAGTGTGCTGGAAAGCTCGTCATGGCGACGGAGCAGAGGCCTTTCC | | |
| 13 | | ACCTGGTGGTGTTCGGCGCCTCTGGCTTCACCGGCCAGTTCGTGACGGAGGAGGTGGCCCGGGAGCAGATAGCCT | | |
| | | CGGAGCAGAGCTCCCGCCTGCCCTGGGCCGTGGCGGGTCGCTCCAAGGAGAAGCTGCAGCAAGTGCTGGAGAAGG | | |
| | | CTGCCCAGAAACTGGGAAGACCATCACTATCATCTGAAGTTGGAGTCATAATCTGTGATATCAGTAATCCAGCCTCA | | |
| | | CTTGATGAAATGGCTAAACAGGCAAAGCTTGTCCTCAACTGCGTAGGACCGTATCGATTTTATGGAGAACCTGTAGT | | |
| | | AAAAGCATGTATTGAAAATGGAACAAGTTGTATTGACATCTGTGGGGAACCTCAGTTTCTGGAACTAATGCATGCGA | | |
| | | AGTATCATGAGAAAGCTGCAgAGAAGGGGGTTTATATCATTGGAAGCAGTGGCTTTGACTCCATCCCAGCAGATCTA | | |
| | | GGAGTGCTATACACCAGGAACCAGATGAACGGTACTTTGACTGCTGTAGAAAGCTTCCTGACAATAAATACAGGACC | | |
| | | TGAGGGGTTGTGTATTCATGATGGAACCTGgAAGCCGGCAATTTATGGtTTTGGCGATAAgGGTAGtTTAAGAAAAC | | |
| | | TACGGAGTGTATCATGTCTGAAACCTGTCCCAATTGTTGGTACAAAGTTGAAAAGAAgGTGGCCAGTCAGCTATTGT | | |
| | | AGAGAGCTGAACTCGTATTCCATTCCTTTTTTGGGATCTGAtATATCTGGCtGGaGcAAAA | | |
| | BGH sequence | ANGNGNAATAANGNGANGGGNTAAAAGAGNAANNAANTANAGTGAGAGAGGGAATGNAGGNNAAATATACNCCTNAN | | |
| | | CCNTATATTGGAAANNNACCTNATATTNNAAACNNNNCTANNNTNATNNANATNANATACNNATNAATNANACNNAANN | | |
| | | ANNANNNTACTANCAAACAACNANNNNAATNNNNNAANNNAAANCTTTANNACANAATCNANNCNNANAAACNATTTCT | | |
| | | TAANCANCANATAANNAAANNNNCNAANANAANTNACANACAANANANCNAATTANNTCATNNANCACCTAANNNCNAA | | |
| | | ANNTNNNTNANTNCNNAATTNNCNNNNATANTTATCNNTACTANTTATCTNANCACTATC | | |
| | Contig | TACCGAGCCCGGATCCCTAGTAACGGCCGCCAGTGTGCTGGAAAGCTCGTCATGGCGACGGAGC | | |
| | | AGAGGCCTTTCCACCTGGTGGTGTTCGGCGCCTCTGGCTTCACCGGCCAGTTCGTGACGGAGGA | | |
| | | GGTGGCCCGGGAGCAGATAGCCTCGGAGCAGAGCTCCCGCCTGCCCTGGGCCGTGGCGGGTCGC | | |
| | | TCCAAGGAGAAGCTGCAGCAAGTGCTGGAGAAGGCTGCCCAGAAACTGGGAAGACCATCACTAT | | |
| | | CATCTGAAGTTGGAGTCATAATCTGTGATATCAGTAATCCAGCCTCACTTGATGAAATGGCTAA | | |
| | | ACAGGCAAAGCTTGTCCTCAACTGCGTAGGACCGTATCGATTTTATGGAGAACCTGTAGTAAAA | | |
| | | GCATGTATTGAAAATGGAACAAGTTGTATTGACATCTGTGGGGAACCTCAGTTTCTGGAACTAA | | |
| | | TGCATGCGAAGTATCATGAGAAAGCTGCAGAGAAGGGGGTTTATATCATTGGAAGCAGTGGCTT | | |
| | | TGACTCCATCCCAGCAGATCTAGGAGTGCTATACACCAGGAACCAGATGAACGGTACTTTGACT | | |
| | | GCTGTAGAAAGCTTCCTGACAATAAATACAGGACCTGAGGGGTTGTGTATTCATGATGGAACCT | | |
| | | GGAAGCCGGCAATTTATGGTTTTGGCGATAAGGGTAGTTTAAGAAAACTACGGAGTGTATCATG | | |
| | | TCTGAAACCTGTCCCAATTGTTGGTACAAAGTTGAAAAGAAGGTGGCCAGTCAGCTATTGTAGA | | |
| | | GAGCTGAACTCGTATTCCATTCCTTTTTTGGGATCTGATATATCTGTTGTGAAAAGGACTCAGC | | |
| | | GTTACTTACATGAAAATTTAGAGGACTCACCAGTTCAGTATGCTGCTTATGTGACGGTGGGAGG | | |
| | | CATCACCTCTGTGATTAAGCTGATGTTTGCAGGACTGTTCTTTTTATTCTTTGTGAAGTTTAGC | | |
| | | ATTGGAAGACAACTTCTCATAAAATTCCCATGGCTCTTTTCCTTTGGCTATTTTTCAAAACAAG | | |
| | | GTCCAACACAAAAACAGATGGATGAGACATCATTTACAATGACATTCTTTGGTCAAGGATACAG | | |
| | | CCATGGCACTTGTGTTGAAAAGAACAAACCAAATATCCGAATCTGCACTCAAGTGAAGGGACCA | | |
| | | GAGGCTGGCTACGTGGCTACTCCCATAGCCATGGTTCAGGCTGCCATGACTTTTCTGAGTGACG | | |
| | | CCTCTGACCTTCCAAAAGGGGGCGGTGTCTTTACACCTGGAGCAGCTTTCTCCAGAACAAAGTT | | |
| | | GATTGACAGACTCAACAAACATGGCATTGAATTTAAGTGTCATTAGCAGCTCCGAAGTCTAAAC | | |
| | | GTTTGAAGACTAACCGAATGATAAAATGCACAAACCGCGTCTGTATTTGGATATGTGAAATTCT | | |
| | | TCTATAAGCCTATCTGACTGTATGTGGACTGTCAAGTTATAAAATAT | | |
| | Identity | >gb\|AF151807.1\|AF151807 Homo sapiens CGI-49 protein mRNA, complete cds | | |
| | | Length = 2127 | | |
| | | Score = 1249 bits (630). Expect = 0.0 | | |
| | | identities = 1130/1293 (87%). Gaps = 4/1293 (0%) | | |
| | | Strand = Plus/Plus | | |
| | | Score = 54.0 bits (27), Expect = 2e−04 | | |
| | | identities = 39/43 (90%) | | |
| | | Strand = Plus/Plus | | |
| Clone # | T7 sequence | AAAGGAAAAACACAGCTNAGCAGATCCAGGCACTAAAGAGAGCTAGCTGCAAGCAGGAGCAGTCAAGAATCTGNGG | | |
| 14 | | TCAGAAGTACTGGAGNGGGCCAGCAGGGCCAGCTTTTTCTACCATGGCAGCCCAAGGCTACGGCTACTATCGCACT | | |
| | | GTCATATTNGCGGCCATGTTNGGAGGCTACAGCCTGTACTATTTCAACCGCAAAACCTTCTCCTTTGTCATGCCCTC | | |
| | | CTTGGNGGATGAGATCGCTCTGGACAAGGACGATTNGGGGCTNATNACAAGCAGCCAGTCGGCAGCCTACGCCATC | | |
| | | AGCAAGTTNGNGAGCGGNGNTCTGTCAGATCAGATGAGCGCCCGCTGGCTCTTCTCCTCTGGGCTGCTCCTGGTNG | | |
| | | GTCTGGTCAACGTAGTCTTCTTATGGNGCTCCACAGNGTCAGCGTTAGCTGCTCTTNGGTTNTCTTAATGGTCCTGG | | |
| | | CACAGG | | |
| | BGH sequence | NAGNATCAAANCTAGCTTNANNGATCTANACAAGNCGGNTNGCCCTCTATATTCTTCCCTTTNGNCCCAGGGNATTCA | | |
| | | NGANAGAGGAGGACTCCTCTCCTCCCTTAGGGACNGAGGNANNGGCAACAATTTNGCCCCCNGCCAAGAAGCCTNG | | |
| | | GGGNACAGCAGAAACCACAGGCCATTAATACTCACTAGGAGATCAGGACCTAGGAGAAGAAGAAGGGTATAGGAGA | | |
| | | CACTCTGAANTNAGGAGNNGCCNGNCNGCCAGAAGGNAGAAACAGAAGCNGAAGGAGTCTAGAACCAAACATCATCA | | |
| | | TTTTAAATAGAGCANGGGAAGGGAGNNGGGACCTNANTAGCCACNGGAAACTTGCAGCACCANGGAGGTTCAGAGA | | |
| | | GACAG | | |
| | Identity | gb\|AF080469\|AF080469 Mus musculus putative glycogen storage disease | | |
| | | type 1b protein mRNA, complete cds | | |
| | | Length = 1923 | | |
| | | Score = 739 bits (373), Expect = 0.0 | | |
| | | Identities = 436/460 (94%), Positives = 436/460 (94%), Gaps = 2/460 (0%) | | |
| Clone # | T7 sequence | ATGGCTGCGCTCTTGCTGAGTGCTGCTCCTTTGGGAACCACAGCTAAGGAGGAGATGGAGCGGTTCTGGAAGAAG | | |
| 15 | | AACACGAGTTCAAACCGTCCTCTGTCTCCCCATTTGACTATGTACAAATGGTCTGTTCCTATGGCACTGTCCGTTTGC | | |
| | | CACCGAGGCTCTGGAATAGCCTTGAGTGGAGGGGTCTCTCTTTTTGGCCTGTCGGCACTGGTGCTTCCTGGGAACT | | |
| | | TTGAGTCGTATTTGATGTTTGTGAAGTCCCTGTGTTTGGGGCCAACACTGATCTACTCGGCTAAGTTTGTGCTTGT | | |
| | | CTTCCCGCTCATGTACCACTCACTGAATGGGATCCGACACTTGCTATGGGACCTAGGAAAAGGCCTGGCAATACCCC | | |
| | | AGGTCTGGCTGTCTGGAGTGGCGGTCGTGGTTCTTGCTGTGTTGTCCTCTGGCGGGCTGGCCGCCCTGTGA | | |
| | Identity | gb\|U31241\|CGU31241 Cricetulus griseus integral membrane protein C11-3 | | |
| | | mRNA, nuclear gene encoding mitochondrial protein, complete cds | | |
| | | Length = 1176 | | |
| | | Score = 242 bits (122), Expect = 3e−62 | | |
| | | Identities = 227/265 (85%), Positives = 227/265 (85%) | | |
| | | gb\|S74803\|S74803\|CII-3 = succinate-ubiquinone oxidoreductase complex II | | |
| | | membrane-intrinsic subunit [cattle, heart, mRNA, 1289 | | |
| | | nt] | | |
| | | Length = 1289 | | |
| | | Score = 186 bits (94), Expect = 1e−45 | | |
| | | Identities = 214/257 (83%), Positives 214/257 (83%) | | |
| Clone # | T7 sequence | CGAGCTCGGATCCACTAGTAACGGCCGCCAGTGTGCTGGAAAGGTGACAGAGGGGAACAAGATGGCGGCGCCAAA | | |
| 16 | | GGGGAAGCTTTGGGTCCAGGCCCAACTGGGGCTCCCGCCGCTGCTGCTGTTGACTATGGCGCTGGCCGGAGGCTC | | |
| | | GGGGACTGCAGCGGCCGAAGCCTTTGACTCGGTCCTGGGAGACACAGCGTCCTGTCACCGGGCCTGTGAGCTGACC | | |
| | | TACCCCTTGCACACCTACCCGAAGGAAGAGGAGTTATACGCATGCCAGAGAGGCTGCAGGCTGTTTTCAATTTGCCA | | |
| | | GTTTGTGGATGATGGGCTTGATTTAAATCGGACCAAGCTGGAATGTGAATCTGCGTGCACAGAAGCATATTCCCAA | | |
| | | CCTGATGAGCAGTATGCTTGTCATCTTGGCTGCCAGGATCAGTTGCCATTTGCTGAACTGAGACAAGAACAACTCAT | | |
| | | GTCCCTGATGCCAAGAATGCATCTCCTCTTCCCTCTGACTCTGGTGAGGTCGTTCTGGAGTGACATGATGGACTCT | | |
| | | GCACAGAGCTTCATAACCTCTTCATGGACTTTTTATCTTCAAGCCGATGACGGAAAAATAGTTATATTCCAGTCTAA | | |
| | | GCCAGAAATTCAGTATGCACCGCAGTTGGAGCAGGAGCCTACAAACTTGAGAGAATCATCTTTAAGCAAAATGTCCT | | |
| | | ATCTGCAGATGAGAAACTCACAAGCACACAGGAACTACCTTGAAGAGGAAGAAAGCGATGGCTTTTTAAGATGTCTA | | |
| | | TCTCTTAACTCTGGATGGATTTTAACCACAACCCTTGTCCTCTCGGTGATGGTGTTGCTCTGGATCTGTTGTGCAGC | | |
| | | TGTTGCTACAGCTGTAGAACAGTATGTTCCCCCTGAGAAGCTGAGTAT | | |
| | BGH sequence | CAGAACATAATTATNGAAATAGATTTTAANGATTTCAATTNAATACAACTGAAAANGTAGAGNCATTAAATAACATTT | | |
| | | CTGCTATAATCCAGAGGACAGTTTGGAGGCCATTTNCGGGCAGAAGCATCACACCCTAAGGNTTCGGNTATTAAGTN | | |
| | | AGANGACTGACGGTGNGCANGNCAGGGGNGGAGCCACACNTGATCAGCTCATAGAANNTCGGTGAANAGAGGAAAN | | |
| | | CANANCACACCCAANTGCACTANCTAANTANTNACAGATATTAGNNTNAATCTCANNTACANCCCAATGNCCATCTTAA | | |
| | | ANTGACTAGAAANNNCCAGGTNAANCTTACANCNAAATANNGCCCTTCATNGANNTATGGTAACCTNCTATNTNGCAT | | |
| | | TTTATAGCNGTNTTCCTTAANGGCCTATNNTTCNANATGNCACNCATNTNTA | | |
| | 4SP6 sequence | TGCATGCTCGAGCGGCCGCCCTTTTTTTTTTTTTTTTTTTTCAGAACATAATTATTCAAATAGATTTTAATGATTTCA | | |
| | | ATTCAATACAACTGAAAATGTAGTGTCATTAAATAACATTTCTGCTATAATCCAGAGGACAGTTTGGAGGCCATTTC | | |
| | | CGGGCAGAAGCATCACACCCTAAGGTTTCGGTTATTAAGTGAGATGACTGACGGGGGGCATGGCAGGGGCGGGGC | | |
| | | CACAATGATCAGCTCATAGAATTTGGCGAAGAGAGGAAAAAAACCAAAACCAATGCAACTAACTAAGTAGCTACAGA | | |
| | | TATTAGTAAAATAAAAATACAACCCAATGTCCATCTTAAATGACTAGAAAAATACAGGTAAAGTCACAGCAAATAAAG | | |
| | | TCTTCACAGAGTTTGGTAACTTTATTTGCATTTTATAGTGATTTCTTAAGGCCTATGTCCAATGAAACCATCTTAAAA | | |
| | | AGCTCTATGAGGAATGGAAGTTTATGTGTCCACGACTCTTTTAAAAAGCTTAGATTTCTGAGTGAGCAAGGTTCACC | | |
| | | TTGGTGGGCAGGGGCCCTGCCTCCTCATGTTCTTCAGTCTGAGACCTAACAATCACAAGAGAAGGAGCTGGGTATC | | |
| | | TGCTCAGCTTTTGTTCATTCATAAATTCCAAGTCACCATAGATACTCAGCTTCTCAGGGGGAACATACTGTTCTACA | | |
| | | GCTGTAGCAACAGCTGCACAACAGATCCAGAGCAACACCATCACCGAGAGGACAAGGGTTGTGGTTAAAATCCATCC | | |
| | | AGAGTTAAGAGATAGACATCTTAAAAAGCCATCGCTTTCTTCCTCTTCAAGGTAGTTCCTGTGTGCTTGTGAGTTTC | | |
| | | TCATCTGCAGATAGGACATTTTGCTTAA | | |
| | | CGAGCTCGGATCCACTAGTAACGGCCGCCAGTGTGCTGGAAAGGTGACAGAGGGGAACAAGATG | | |
| | | GCGGCGCCAAAGGGGAAGCTTTGGGTCCAGGCCCAACTGGGGCTCCCGCCGCTGCTGCTGTTGA | | |
| | | CTATGGCGCTGGCCGGAGGCTCGGGGACTGCAGCGGCCGAAGCCTTTGACTCGGTCCTGGGAGA | | |
| | | CACAGCGTCCTGTCACCGGGCCTGTCAGCTGACCTACCCCTTGCACACCTACCCGAAGGAAGAG | | |
| | | GAGTTATACGCATGCCAGAGAGGCTGCAGGCTGTTTTCAATTTGCCAGTTTGTGGATGATGGGC | | |
| | | TTGATTTAAATCGGACCAAGCTGGAATGTGAATCTGCGTGCACAGAAGCATATTCCCAACCTGA | | |
| | | TGAGCAGTATGCTTGTCATCTTGGCTGCCAGGATCAGTTGCCATTTGCTGAACTGAGACAAGAA | | |
| | | CAACTCATGTCCCTGATGCCAAGAATGCATCTCCTCTTCCCTCTGACTCTGGTGAGGTCGTTCT | | |
| | | GGAGTGACATGATGGACTCTGCACAGAGCTTCATAACCTCTTCATGGACTTTTTATCTTCAAGC | | |
| | | CGATGACGGAAAAATAGTTATATTCCAGTCTAAGCCAGAAATTCAGTATGCACCGCAGTTGGAG | | |
| | | CAGGAGCCTACAAACTTGAGAGAATCATCTTTAAGCAAAATGTCCTATCTGCAGATGAGAAACT | | |
| | | CACAAGCACACAGGAACTACCTTGAAGAGGAAGAAAGCGATGGCTTTTTAAGATGTCTATCTCT | | |
| | | TAACTCTGGATGGATTTTAACCACAACCCTTGTCCTCTCGGTGATGGTGTTGCTCTGGATCTGT | | |
| | | TGTGCAGCTGTTGCTACAGCTGTAGAACAGTATGTTCCCCCTGAGAAGCTGAGTATCTATGGTG | | |
| | | ACTTGGAATTTATGAATGAACAAAAGCTGAGCAGATACCCAGCTCCTTCTCTTGTGATTGTTAG | | |
| | | GTCTCAGACTGAAGAACATGAGGAGGCAGGGCCCCTGCCCACCAAGGTGAACCTTGCTCACTCA | | |
| | | GAAATCTAAGCTTTTTAAAAGAGTCGTGGACACATAAACTTCCATTCCTCATAGAGCTTTTTAA | | |
| | | GATGGTTTCATTGGACATAGGCCTTAAGAAATCACTATAAAATGCAAATAAAGTTACCAAACTC | | |
| | | TGTGAAGACTTTATTTGCTGTGACTTTACCTGTATTTTTCTAGTCATTTAAGATGGACATTGGG | | |
| | | TTGTATTTTTATTTTACTAATATCTGTAGCTACTTAGTTAGTTGCATTGGTTTTGGTTTTTTTC | | |
| | | CTCTCTTCGCCAAATTCTATGAGCTGATGATTGTGGCCCCGCCCCTGCCATGCCCCCCGTCAGT | | |
| | | CATCTCAGTTAATAACCGAAACCTTAGGGTGTGATGCTTCTGCCCGGAAATGGCCTCCAAACTG | | |
| | | TCCTCTGGATTATAGCAGAAATGTTATTTAATGACACTACATTTTCAGTTGTATTGAATTGAAA | | |
| | | TCATTAAAATCTATTTGAATAATTATGTTCTGAAAAAAAAAAAAAAAAAAAAGGGCGGCCGCTC | | |
| | | GAGCATGCA | | |
| | Identity | >gb\|AF116911.1\|AF116911 MUS musculus thymic dendritic cell-derived | | |
| | | factor 1 mRNA, complete cds | | |
| | | Length = 1513 | | |
| | | Score = 2533 bits (1278), Expect = 0.0 | | |
| | | Identities = 1412/1459 (96%) | | |
| | | Strand = Plus/Plus | | |
| | | >ref\|NM004872.1\| Homo sapiens mouse tropomyosin homolog (HSPC001) mRNA | | |
| | | Length = 1138 | | |
| | | Score = 1134 bits (572), Expect = 0.0 | | |
| | | Identities = 888/992 (89%), Gaps = 1/992 (0%) | | |
| | | Strand = Plus/Plus | | |
| | | Score = 127 bits (64), Expect = 1e−26 | | |
| | | Identities = 92/100 (92%), Gaps = 1/100 (1%) | | |
| | | Strand = Plus/Plus | | |
| | Description | PI-FAGS: Apoptosis | | |
| Clone # | T7 sequence | AAAGGTACGAAGCTAGGGAAGATATTCGCGTGGCTAAATCTGCACGTGGAAGGAGCATTAACTTGGCCCTTTCTTA | | |
| 17 | | TAGAGGACGGCAGGCCTTGAAAGCCATTGGTCTGGAAGATCAGATCGTTTCCAAAGGTGTGCCCATGAAAGCCAGA | | |
| | | ATGATCCACTCTCTTTCTGGAAAGAAGTCTGCAATTCCCTATGGGAACAAGTCACAGTATATCCTTTCAATAAGCAG | | |
| | | AGAAAACTTAAACAAGGACCTGCTGACTGCCGAGGAGTCCTATGCGAATGCGAAGGTGCACTTNGGCCACAAGCTG | | |
| | | TCGAAANGCATTCCGGAGGAAGGGGTACTCACAGAGCTCGGACCTGACAAGGTTCCCCGAGATGTCACATGTGACC | | |
| | | TTG | | |
| | BGH sequence | ANTTTTTTTTTTTTTTTTTTTTTTTTTTTTTTTTTTTTTTTTTTTTTTTNAAAANGGNANAANGNTTTNGNAAANTTTN | | |
| | | GNNGGNNNGGGNAAANAAACCNAGGGNCNNANGNCGGNAAAAAAAGNNTTTTNGCNNAGGGNNNNANNNNTNACCNG | | |
| | | NAAANNAANGNNTTTNNTTTNCNTACCCCTNAAAAANAAANNGGNAAANCCCANGGNNGNTTTNGANANNANGGNAAA | | |
| | | NNCCAANTNGNTTAANNTTAANTTCCNNGGGTTNGGNTTTTNNAGGNAANNAANANGGGNNNTNTNAAAANNGNANNA | | |
| | | NGAAAANAANTTTAANGGGTCCNANNNACCCNNTTNGGAAAAAANNAAATNGNAANGGGNANTTGGGCNNTGGNCTT | | |
| | | NGNNGACNAAGTNAANTTTNGGNTNNGNCCGGGGGGNANNNANGGNNCCCCTTTTGNAAGNNTNAANGGGNNAAAAN | | |
| | | GGGGNCCCANNNTTTNGGAAAA | | |
| | Identity | gb\|AF056031\|AF056031 Rattus norvegicus kynurenine 3-hydroxylase mRNA, complete cds | | |
| | | Length = 1733 | | |
| | | Score = 468 bits (236), Expect = e−130 | | |
| | | Identities = 342/378 (90%), Positives = 342/378 (90%) | | |
| | | emb\|Y13153\|HSKYNU3MO Homo sapiens mRNA for kynurenine 3-monooxygenase | | |
| | | Length = 1999 | | |
| | | Score = 222 bits (112), Expect = 4e−56 | | |
| | | Identities = 308/374 (82%), Positives = 308/374 (82%) | | |
| | Description | DNA ladder | | |
| Clone # | T7 sequence | AAAGCGACTGCACAGNGAAGCCCTCTGTTACCTGTGTCGATCAAGACCTNAAACCCCAGAGGAACTTCGTCATCAAC | | |
| 18 | | ATGACTTGCAGGTTTNGCTGGCAGCTTCCTGAAACAGACTACGAGTGTTCAAATTCCACCACCTGCATGACCGNGGC | | |
| | | TNGCCCTCGGCAGCGCTATTTCGCCAACTGCACCGNGCGTGACCACATTCAGTGCCTGGGCAACCGGACTTTCCCTA | | |
| | | AGCTGCTGTACTGCAACTGGACAGGNGGCTACAAGNGGTCGACAGCCCTGGCTCTNAGCATCACCCTNGGGGGGNT | | |
| | | AGGAGCCGATCGCTTCTACCTGGGCCAGNGGCGAGAAGGCCTCGGCAAGCTCTTCAGCTTTGGCGGCCTGGGAATA | | |
| | | TGGACCCTAATCGATGTCTNGCTGATNGGAGTAGGCTATGNGGGACCAGCGGATGGCTCTTTGTACATTTAGCCGA | | |
| | | GGTTATGTGCTTCAGAGAGCAGNGTAGAGTCCTGNGTGTGGAGATGGATGCGGAGTGAGAG | | |
| | BGH sequence | CAGNGTTCGATTTCTTTATTTTACCTTCATCAAGGCAAGCCAAGTACAGATGCTGTACATTAAAAACATAAATCCCCC | | |
| | | TNTCACACCGCATCCATNTCCACACACAGGACTCTACACTGCTCTCTGAAGCACATAACCTCGGCTAAATGTACAAAG | | |
| | | AGCCATCCGCTGGTCCCACATAGCCAANTCCAATCAGCAAGACATCGATTAGGGTCCATATTCCCAGGCCGCCAAAG | | |
| | | CTGAAGAGCTTGCCGAGGCCTTCTCGCCACTGGCCCAGGTAGAAGCGATCGGNTCCAAACCCCCCANGGGTGATGC | | |
| | | TGAGAGCCAGGGCTGTCGACCACTTGNAGCCACCTGTCCAGTTGCAGNACAGCAGCTTAGGGAAAGTCCGGTTGCC | | |
| | | CAGGCAGTGAATGTGGNCACGCACGGTGCAAGTTGGCGAAATAGCGCTGCCGAGGGCAAGCCACGGCATGCAGGT | | |
| | | GGTGGAATTTGAACACTCGNAGTCNGTTTCAGGAAGCTGCAGCAAAAGTGCAGGCATGTTGATGACNAAGTCCTTG | | |
| | | GNGATTGAGGCTTGACGACACAGNTAACAGAGGGCTTACTTNGCAAGCCGNTTTCAGCACAATTGGCCGCNCGGAA | | |
| | | CTAGGGGAANCCGAGGTTTGGGACCCAAGCTTGGGGNCTCCCTTAAAGGGGNGGTCCGAAATNAAATTTCCGANAA | | |
| | | GGCCNGGNAANCCAAGNGGGGNNCTTNTAGGTTAGCCCCGAAGNGGCNTCTGCTTAANNTAGGACCTTTCCACCGG | | |
| | | AAACGCCTAACGGCCCAATTGGGGNNAAAAGGGNCCCGNGTTGTACCCACAATTTTGNAAAGNCCCNTCGATTGGG | | |
| | | GCCAAACAANCTCCCATGGCNTCAAANGGGGGAATGNAAACCCGGGGTAAANCGTTCCCCCCCATGGGNTGCNAACN | | |
| | | GNAACAGGNAAGGNGNNTAAACNNGCNNCCCCNCGAGCCCAAGGNTCTGGCAAAGNGNGGGCNAAACCATTCCCNAA | | |
| | | NGGGATNAAAAAACTNGTCCCCCAGGGGTCNAAACCCCT | | |
| | T7 contig | GCAACAATTCGAGCTGCTGTGACAGAGGGGAACAAGATGGCGGCGCCAAA | | |
| | | GGGGAAGCTTTGGGTCCAGGCCCAACTGGGGCTCCCGCCGCTGCTGCTGT | | |
| | | TGACTATGGCGCTGGCCGGAGGCTCGGGGACTGCAGCGGCCGAAGCCTTT | | |
| | | GACTCGGTGCTGGGAGACACAGCGTCCTGTCACCGGGCCTGTCAGCTGAC | | |
| | | CTACCCCTTGCACACCTACCCGAAGGAAGAGGAGTTATACGCA:TGCCAG | | |
| | | AGAGGCTGCAGGCTGTTTTCAATTTGCCAGTTTGTGGATGATGGGCTTGA | | |
| | | TTTAAATCGGACCAAGCTGGAATGTGAATCTGCGTGCACAGAAGCATATT | | |
| | | CCCAACCTGATGAGCAGTATGCTTGTCATCTTGGCTGCCAGGATCAGTTG | | |
| | | CCATTTGCTGAACTGAGACAAGAACAACTCATGTCCCTGATGCCAAGAAT | | |
| | | GCATCTCCTCTTCCCTCTGACTCTGGTGAGGTCGTTCTGGAGTGACATGA | | |
| | | TGGACTCTGCACAGAGCTTCATAACCTCTTCATGGACTTTTTATCTTCAA | | |
| | | GCCGATGACGGAAAAATAGTTATATTCCAGTCTAAGC | | |
| | possible ORF | MAAPKGKL WVQAQLGLPP LLLLTMALAG GSGTAAAEAF | | |
| | | DSVLGDTASC HRACQLTYPL HTYPKEEELY ACQRGCRLFS ICQFVDDGLD | | |
| | | LNRTKLECES ACTEAYSQPD EQYACHLGCQ DQLPFAELRQ EQLMSLMPRM | | |
| | | HLLFPLTLVR SFWSDMMDSA QSFITSSWTF YLQADDGKIV IFQSK | | |
| | Identity | >gb\|AW109849.1\|AW109849 MT2475 mouse liver, dioxin treated Mus musculus | | |
| | | cDNA clone MT2475 3′ | | |
| | | Length = 617 | | |
| | | Score = 833 bits (420), Expect = 0.0 | | |
| | | Identities = 489/517 (94%), Gaps = 2/517 (0%) | | |
| | | Strand = Plus/Minus | | |
| | | >gb\|AA277327.1\|AA277327 va81e12.r1 Soares mouse NML Mus musculus cDNA | | |
| | | clone IMAGE:737806 5′ similar to WP:C02F5.3 CE00039 GTP-BINDING PROTEIN; | | |
| | | Length = 553 | | |
| | | Score = 789 bits (398), Expect = 0.0 | | |
| | | Identities = 483/514 (93%), Gaps = 5/514 (0%) | | |
| | | Strand = Plus/Plus | | |
| | | >gb\|AI646762.1\|A1646762 ub65e01.x1 Soares_mammary_gland_NMLMG Mus musculus | | |
| | | cDNA clone IMAGE:1382616 3′ similar to WP:C41D11.5 CE08662 ENDONUCLEASE; | | |
| | | Length = 529 | | |
| | | Score = 741 bits (374), Expect = 0.0 | | |
| | | Identities = 425/449 (94%) | | |
| | | Strand = Plus/Minus | | |
| | Description | DNA ladder | | |
| Clone # | T7 sequence | AAAGCTGGCTGGAATCCTGGTAGGAGCCGCAGTGNGNGGCCATGCCTCAGATAGGTTNGGGCGCAGAAGGGTGCT | | |
| 19 | | GACCTGGAGCTATCTTCNGGNGTCCGNGTCCGGCACAGTAGCTGCCTTCATGCCCACCTTCCCCCTCTACTGNCTGN | | |
| | | TNCGTTTCCTGCTGGCCTCTGCAGNGGCAGGAGTTANGATGAACACAGCCA | | |
| | BGH sequence | AAAAATTATTTATTNGNGTGTATCACCGGGGGCGGGNATGTGTCCATGCGGAGCTCANAGGACAACTTTGTGAAGT | | |
| | | CTGTTGTCACATGAGTTTCAGAAATTTAGGCCCGGAGGCAGGNGTCTTTACCCGCCGTCGCCAGCCCTGCCCTTTCC | | |
| | | TCCTCCTCCTCCTCTTCCTCCTCCTCCTCCTCTA | | |
| | Identity | dbj\|AB005451\|AB005451 Mus musculus mRNA for RST, complete cds | | |
| | | Length = 1779 | | |
| | | Score = 305 bits (154), Expect = 2e−81 | | |
| | | Identities = 185/199 (92%), Positives = 185/199 (92%) | | |
| | | LOCUS AB005451 1779 bp mRNA ROD Dec. 17, 1997 | | |
| | | DEFINITION Mus musculus mRNA for RST, complete cds. | | |
| | | ACCESSION AB005451 | | |
| | | NID g2696708 | | |
| | | KEYWORDS RST; renal-specific transporter. | | |
| | | SOURCE Mus musculus 8-week-old male kidney cDNA to mRNA, clone:K14D2. | | |
| | Description | DNA ladder | | |
| Clone # | T7 sequence | AAAGGGGACGGAGACCTTCAGCGTGGAATCTATATCCAAGAATGGAATCTGTCTGGAGATGGGCCCACAGCCTCAG | | |
| 20 | | GGCGTGCTGCGGGCCGACCTGTTCTCCCGGATGCGAGCTCTGGNGGCATCCATTCTGGACTTCATCGAGCTCTTNA | | |
| | | ACCAAGGCATGGACTTACCCGCCTTTGAGATGGATATCTACAGGAACTTGGGCAGNGNGGACTTCCCACGCACTGC | | |
| | | GGNNGGNGACCTGGCTGGCACTGNGCACCCTCAACTGCAGGACCATGACTTTGAGCCACTGAGGCCTGGAGAACCC | | |
| | | ATCTTCAAGCTTTTCAGCGGAGAAGANGTACTGTATNAGGGGGACTCCATTGNGTACCCTGNNNTCATTNNTGAGG | | |
| | | CTGCCCTATT | | |
| | BGH sequence | GGGGCACAGTCTATTTTATATAATGAGATCAGTTGTCTGGAAAGGGATATGGNGTCCAGAGTGAGATAGGGGGACCC | | |
| | | TGGGCTTGGGACCCATTGTTCAGATAGGAAAACTGAGATCAGGCCAGGTATGATTNGGGTTAGGGAGTCTGGGNG | | |
| | | CAGCGGGGGGTCAACCTNAGCAGGGCAGGCACTGNGACCCTGATCTTCTCAGACTTCAGGAATGCCACGTGCTTCT | | |
| | | CATAATAGGCAGCCTNATTAATGAACACAGGGTACACAATGGAGTCCCCCTCATACAGTACGTCTTCTCCGCTGAAA | | |
| | | AGCTTGAAGATGGGTTCACCAGGCCTNAGNGGCTCAAAGTCATGGTCCTGCAGTTGGGGTGCACAGTGCCAGCCA | | |
| | | GGTCACCATCCGCAGNGCGTGGGAAGTCCACACTGCCCAAGTTCCTGTAGATATCCATCTCAAAGGCGGGTAAGTC | | |
| | | CATGCCTTGGTTGAAGAGCTCGATGAAGTCCAGAATGGATGCCACCAGAGCTCGCATCCGGGAGAACAGGTCGGCC | | |
| | | CGCAGCACGCCCTGAGGCTGTGGGCCCATCTCCAGACAGATTCCATTCTTGNATATAGATTCCANGCTGAAGGTCTN | | |
| | | CGTCCCCTTTCCAGCACACTGGCGGCCGATACTAGAGGATCCGAGCTNNGTACCAAGCTTGGGCTCCCTAAGGGAG | | |
| | | TCNNATANTTTNNATAGCCNGTAAGCAGTGGGTNTCTGNTA | | |
| | Identity | gb\|AA109018\|AA109018 mp37f03.r1 Barstead MPLRB1 Mus musculus cDNA | | |
| | | clone 571421 5′ | | |
| | | similar to SW:ACY2_HUMAN P45381 ASPARTOACYLASE; | | |
| | | Length = 535 | | |
| | | Score = 622 bits (314), Expect = e−177 | | |
| | | Identities = 340/352 (96%), Positives = 340/352 (96%) | | |
| Clone # | T7 sequence | AAAGGGCGGCGGCAGCAGCTCCCGGGGCTCNTGCTCTGCTCCGCCTCGGCCCCGGAGCGAGGGGCGGAGAGCCGC | | |
| 21 | | GCGCTNGCCTTAGTCCGAGCCGTCACCCTCCCCGCGNTCCCCGCTTTNCCGGCCCGCCCGAGGCCGNCCGCCCCGTC | | |
| | | CCCGCCGCNCCGNAGCCNGGCCGTGCCCCGCCGNCGNCATGNGCTGCCTCGGCAACAGTNAGANCCGAGGANCAGC | | |
| | | GNANCTGAGGAGAANGCGCAGCGCGAGGNCAACAAANAGATCTGAGAAGCAGCTGCAGAAGGACAAGCAGGTCTAC | | |
| | | CGGNTCACGCACCGNCTGCTGCTGCNGGNNGCTGGAGAGNCTGGCAAAAGCACNATTGTGAAGCANATGAGGATCC | | |
| | | TGCATGNTAATGNGTNTAANNGANAGGGCGGTAANAGGACCCCGCAGGCTGCAAGGAGCAACANTTGATGGTNANA | | |
| | | AGGCCACTANAGNGCNNGACNTTNNANNCNATCTGNANGANGCCATNNAAACCANTGTGGCCNACATNAGCANCCTT | | |
| | | GCTGCCCCCTGCNNAGNTGNCCAACCCTGCNANNNAGTTCANAGNTGNNCTACATTCTGNGCNTCATNAACNGTGCN | | |
| | | CNNACTTTTCACTTTCCACCTNNNTTNTATNAAGCCATNNCCAAGGCTTCTNT | | |
| | BGH sequence | GCTCATTTTTAATTTTTATTGATTTTTTAATGCTGCACAACACAATATTTATTTCATTTTGAATTTCATTTATTTCTTT | | |
| | | ATTTCTGTNGCTGCTTTTATTTTATTTACTGAAAGTGAGAGGGAACTTTNGNGGCCTTTTTTTTCTTTTTCTTCTGT | | |
| | | AGGCCGCCTTAAGCTTACTAAATTTGGAACATCTAAGCAAGCTGAAGGGAAGAGGGGTTTTTCAGAATCACTGGGG | | |
| | | GAAAAAGGAAAGGNNGCGGAGTTGATCATGCCCTATGGNGGGNGACCAACTGCTTGTACAATTACGTTTCACTCTT | | |
| | | AATTAATNGNGCTTAAGGCTGAATTAAATTTGGGNGNTCCCTTCTTAGAGCAGCTCTGNATTGGCGGAGATGCATG | | |
| | | CGCTGGATGATGTCACGGCAGTCGTTGAAGACACGGCGGATGTTCTCAGNGTCCACGGCGCAGGTAAAGTGAGGG | | |
| | | TAGCAGTNGNGGCGCCCATNTCCACTAGCAGTGCTGNTTTTCAGAAACTNATCCCTNATGAAGTNCTT | | |
| | Identity | emb\|Y00703\|MMGTPAMU Mouse uncoupled S49 cells mRNA for stimulatory | | |
| | | GTP-binding protein alpha subunit | | |
| | | Length = 1359 | | |
| | | Score = 276 bits (139), Expect = 5e−72 | | |
| | | Identities = 242/277 (87%), Gaps = 6/277 (2%) | | |
| | | Strand = Plus/Plus | | |
| | | gb\|M12673\|RATGNPAS Rat guanine nucleotide-binding protein G-s, alpha | | |
| | | subunit mRNA complete cds. | | |
| | | Length = 1708 | | |
| | | Score = 266 bits (134), Expect = 5e−69 | | |
| | | identities = 224/255 (87%). Gaps = 5/255 (1%) | | |
| | | Strand Plus/Plus | | |
| | | Score = 163 bits (82), Expect = 5e−38 | | |
| | | Identities = 89/92 (96%) | | |
| | | Strand = Plus/Plus | | |
| | | gb\|M17525\|RATBPGTPD Rat GTP-binding protein (G-alpha-8) mRNA, complete cds. | | |
| | | Length = 1738 | | |
| | | Score = 266 bits (134), Expect = 5e−69 | | |
| | | identities = 224/255 (87%), Gaps = 5/255 (1%) | | |
| | | Strand = Plus/Plus | | |
| | | 9b\|AF116268\|AF116268 Mus musculus G-protein XLAS (Xlas) mRNA, | | |
| | | alternatively spliced, complete cds | | |
| | | Length = 2655 | | |
| | | Score = 609 bits (307), Expect = e−172 | | |
| | | identities = 357/378 (94%), Gaps = 1/378 (0%) | | |
| | | Strand = Plus/Minus | | |
| Clone # | T7 sequence | AGCGGACGTTTGTGCCGGGACATGGCCGCTGCGGATGCCGGGTCCTTGAGCTGAGCGCTTGCTGCCGGAGCCAAC | | |
| 22 | | CTCTGCCGTCAACCGTCCGCGCGGGCTGGGCCCAGGCGCCGGGACGGCCAAGATCCGCCGAGGAAGCTGAGGCAG | | |
| | | CTATAGAACGCCGCCGCGGCGGGCGCATGGCGTCCATCTTGCTCAGGAGCTGCCGGGGCCGGGGGCCTGCCCGCC | | |
| | | TCGCGCCACCTCGGGCCGNCTCCCCGCGGGGTAGTCTGAGGGATCGAGCTTGTCTCAGCTGTACCAGGACCTGGG | | |
| | | GTTGACGAGCCGTGAGAGTGTTCTGTCTCGTTGCTGTACTCCAGCCCACCCTGTGTACCTCTGCTTCAAAGGTGAG | | |
| | | CCCCTNAGCTGTTGGACTCAGAGGCCTGAGTGCCAGGGCACCGCAGCAAGAACAACATGGACACCTGCCTCTGCAA | | |
| | | GGCTGGNGGTTACGGGACCTCAGTACCTTCCTGTGCGGGGCTGGCACTCATCATCTTCGCTAGGAGAGGACTCTGT | | |
| | | GATAGAGAAGTCCCTTAAGTCCTTAAAAGACAAGAATAANAAGCTGGANGAGGGCGGNCCCGTGTACAGCCCGGNC | | |
| | | GCGCAGGTGGTGGTGAGGAAATCCCTNGGGCAGAAGGTACTGGATGAGCTGAGGCACTACTACCATGGCTTTCCG | | |
| | | CCTGCTCTGGGATNGACACCAAAGANCGCTTGNCCGGATGCTCTGGCGCATNCTTNATGGGNCATACGCTTAGGCCG | | |
| | | NCCGGGAGCCNCAGGCAGNTTNTTCCNGAATTGTGCCGGACCTNTTNCCGCTAAGNGGCCCTTTCTTGGGGGNNCC | | |
| | | NGGNGGGGGCCCNTTAATGGNA | | |
| | BGH sequence | 1st sequencing: | | |
| | | CATTATGGAAGTTTCTGATTNATTGCAGACAAAATATTANATTTGCCANTAAGAATCACCTCAAAGCAATCAGTCTTG | | |
| | | NGNGTCATGCCCCTGNGGCTGGCACGGCATGATGAAGGCAGACCTGNGGGCCAGAAGCTGGCAGCTCTAATTCTTC | | |
| | | ACTTCTGCGGCCTCTTTCTCAGCCTTCTCCTTGGCCTTCTCTTTCTCTTCTATCTTTTCTTCTTTCTCTAGNGNGGCC | | |
| | | ACAATTTCAGCTACCTGGGNGGNAGAGATCTGAACATCTTCTTTGTTCACCAAGTCGATTACCTTGACAAGGTCATC | | |
| | | GATGTTGATATNGCCGNCCTTATTATCATCCAGGGCTGAGGTCAAACTGATGAGCTTGAGCTCTGGAATGNGCTNG | | |
| | | ATTTGCTTCATGGCGCTGATGAGCTCAGNGANGCTGATGACACTCTCCCCTGNAGGAGNGCTCTGGCTGGGGCCCA | | |
| | | GNTTGCCATCCTGATGAGAGGACTCCAGCTGTGTGATCAGGCCATGGATCTGCCCGATCATCTGCTGNNCTGNCTNT | | |
| | | GACAGCGTCTTGCTCGCTGCAGAATCTTCTATGNNCTTTCTCCTCACCCGNCTNTTNNANGGTCCTTCTTTGAACTCC | | |
| | | TGTANNNCCTCACNTGAGGCCTGGNCGCCCTCTTTTGAGCAGGCNTCCAGGCTTCCTCCTTTCTCCCTTGGCNCNGG | | |
| | | GGACNNTCCCTTNCGCNTCCTGCNNCTTT | | |
| | | 2nd sequencing | | |
| | | CATTATGGAAGTTTCTGATTNATTCCANACAAAATATTANATTTGCCACTAANAATCACGTNAAAGCAATCACTNTTG | | |
| | | NGNGTNATGCCCCTGNGGCTGGCACGGCATGATGAAGGCANACCTGNGGGCCAAAAGCTGGCAGNTNTAATTGTTC | | |
| | | ACTTCTGCGGCCTNTTTNTNAGCCTTCTCCTTGGCCTTCTCTTTCTCTTCNATCTTTTCTTGTTTCTCTAGNGNGGCC | | |
| | | ACAATTTCAGCTACCTGGGNGGNANANATCTGAACATNTTCTTTGTTCACCAAGTCGATTACCTTGACAAGGTCATC | | |
| | | GATGTTGANATTGCCGNCCTTATTATCATCCAGGGCTGAGGTCAAACTGATGAGCTTGNGCTNTGGAATGNGCTTG | | |
| | | ATTTGCTTNATGGCGCTGATGAGCTCAGTGATGCTGATGACACTNTCCCCTGTTGGAGNGCTCTGGCTGGGGCCCA | | |
| | | GCTTGCCATCCTGTTGAGNGGTCTCCANCTGTGTGATCAGGCCATCGATCTGCCCGANCATCTGCTGCACTCGCTTT | | |
| | | GACAGCCTCTTGCTCGCTGCAAATTCTTCTATGNACTTCTCCTCACCCGTCTTTGAAAGTTCCTTCTTGATCTCCTGT | | |
| | | AAGTCCTCACTGTAGNCCTGGACGNCCTCTTTGAGCAGNTCCAGCTCCTCCTTNTNCTTGGTCAGGGACTTCTTCTG | | |
| | | CTNCTGCAGCTTGGAACAGGCGTACTGAGGATGTNAANCTCCNCTTGGNANCTNTTTACCCTTTAGACGTTTCAAAC | | |
| | | ANGGGCCGAATCCGNNAGGANCTTTGNGGGCAGGAANANAAAAN | | |
| | Identity | gb\|AF061026\|AF061026 Mus musculus leucine zipper-EF-hand containing | | |
| | | transmembrane protein 1 (Letm1) mRNA, complete cds | | |
| | | Length = 3480 | | |
| | | Score = 549 bits (277), Expect = e−154 | | |
| | | Identities 336/360 (93%), Gaps = 1/360 (0%) | | |
| | | Strand = Plus/Minus | | |
| | | Score = 283 bits (143), Expect = 2e−74 | | |
| | | Identities = 157/164 (95%) | | |
| | | Strand = Plus/Minus | | |
| Clone # | T7 sequence | AAAGGGCGGCCATGGACCGCTTCGNGNGGACCAGNGGCCTCCTGGAGATCAACGAGACCCTGGTTATCCAGCAGCG | | |
| 23 | | CGGNGNGCGCGTCTACGACGGCGAGGAGAAGATAAAATTNGATGCCGGGACTCTTCTTCTTAGTACACACCGGCTG | | |
| | | ATTNGGAGAGACCAGAAGAATAATGAGNGCTGNATGGNNATTNCCCTGTCTTAGATNGNGNTCATCGAGGAGCAGG | | |
| | | CAGCTNNAATCGGGAAGANNNNCAAAATNGNGGNTCACCTGCACCCAGTTCCTTTTNACAAA | | |
| | BGH sequence | CNANCNCATTCTNGGNNAATTNGGGNTAATTTTTNANCTNAGNGTCNNGAGACCTTGNNAAAANGCAAGNTNATNGC | | |
| | | CATAAAGCATTTCAGGNNCAAAATTNTNAGTNCTGGGNCANAAANAAATTTGGANAAAACCGAANGCNTTCCANGGN | | |
| | | GCNGTNTCGGAAAAGGGGNCCNATTTTNTTTGNANGGNGCNCCNTTTNTTNACCCANANGGNCAGACNTTCCCNNAN | | |
| | | GCTNGGNAANNTTTTNGGANGTNAAGGNCCCNNTTTTNGAANCCGTTTCNAGGGCCGNGGCCNCNATTTCCTTTTCC | | |
| | | TNGGGNGTT | | |
| | Identity | gb\|AA086895\|AA086895 mk19c02.r1 Soares mouse p3NMF19.5 Mus musculus cDNA | | |
| | | clone 493346 5′ similar to WP:F17C11.8 CE05655 | | |
| | | Length = 480 | | |
| | | Score = 353 bits (178), Expect = 9e−96 | | |
| | | Identities 250/281 (88%), Positives = 250/281 (88%). Gaps = 1/281 (0%) | | |
| | | gb\|AA881548\|AA881548 vx20b03.r1 Soares 2NbMT Mus musculus cDNA clone | | |
| | | 1264973 5′ | | |
| | | Length = 454 | | |
| | | Score = 331 bits (167). Expect = 3e−89 | | |
| | | Identities = 226/253 (89%), Positives = 226/253 (89%) | | |
| | | gb\|AA073042\|AA073042 mm78b04.r1 Stratagene mouse embryonic carcinomaRA (#937318) | | |
| | | Mus musculus cDNA clone 534511 5′ similar to WP:F17C11.8 CE05655; | | |
| | | Length = 429 | | |
| | | Score = 321 bits (162), Expect = 3e−86 | | |
| | | Identities = 245/277 (88%), Positives = 245/277 (88%), Gaps = 5/277 (1%) | | |
| Clone # | T7 sequence | AAAGNGGGGATCCTGGAGACAGTTCTGGNGCAGAGTTCCAGTACCTCTACATCCATCATTGTCAGCATGGTCTCCTC | | |
| 24 | | CGGCTNGTNGGAGGNGAGCTCCGCCATTCCGATCATGATGGGCTCCAACATGGGAACCTCTGTCACCAACACCATTG | | |
| | | NGGCCCTGATGCAGGCAGGGGACAGGACTGACTTNAGGCGGGCTTTNGCAGGGGCGACCGNGCATGACTGTTTTA | | |
| | | ACTGGCTGTCNGTTCTGGNCCTACTGCCCCNGGAGGCTGCCACGGGCTACCTACACCATGTCACCGGGCTNGAGGN | | |
| | | NGCTTCCTTNAACATCCNAGGNGGCCGNGATGCCCCCGACCTTCTCAAAGTCATCACAGAGCCCTTNANAAGACTCA | | |
| | | TCATCCAGCTGGACAAGTGTGNGATNACCAGCATNGCCGAGGGGGATNAGTNCCTGAGGAATCACAGTCTCANTC | | |
| | Identity | gb\|U22465\|MMU22465 Mus musculus Na/Pi-cotransporter (NaPi-7) mRNA, complete cds | | |
| | | Length = 2423 | | |
| | | Score = 718 bits (362), Expect = 0.0 | | |
| | | Identities = 421/449 (93%), Positives = 421/449 (93%) | | |
| | | gb\|L33878\|MUSSPT Mus musculus renal sodium phosphate (Na+/Pi) transporter mRNA, | | |
| | | complete cds | | |
| | | Length = 2401 | | |
| | | Score = 718 bits (362), Expect = 0.0 | | |
| | | Identities = 421/449 (93%), Positives = 421/449 (93%) | | |
| | Description | DNA ladder | | |
| Clone # | T7 sequence | AGGGCGCCAGCTGAAGACGCGGGACTTAAAGNGCGTAGCCAGAACCCAGGCACCAGNGTGTCCATTGTCCAGAACT | | |
| 25 | | CATCTGAAAAACTGCCACAGGAATTGCTTCTCTGCTCCAGGCTGGTCACTGAACAGGTTGCTCCAGGACCTGCAGAA | | |
| | | TGGGGGCAGGCTGNGTCAAAGTCACCAAGTATTTCCTCTTCCTCTTCAACTNGCTGNTCTTTATCCTGGGNGCTGAG | | |
| | | ATCCTGGGCTTCGGNGAGNGGATTCTTGCAGACAAGAACAGCTTCATTTCCGTCCTACAAACCTCATCCAGCTCGCT | | |
| | | GCAGGNGGGGGCTTACGTCTTCATCGGAGNGGGCGCCATCACCATAGNGANGGGCTTCCTGGGCTGTATCGGAGC | | |
| | | TGTCAATGAGGNCCGCTGCTTGCTGGGTCTGTACTTNGTCTTCCTTCNGCTGATCCTNATCGCACAGGTGACCGTA | | |
| | | GGGGTCCTCTTCTACTTCAACGCTGACAAGCNGAAGAAGGAGATGGGGAACACAGNGATGGACATCATTCGCAACT | | |
| | | ACACTGCCAATGCCACCAGTAGCCGCGAGGAGGCCTGGGACTACGTGCAGGCGCAGGTCAAGNGCTGTGGCTGNG | | |
| | | TCAGCCACTACAACTGNACAGAGAACNAGGAGCTCATGGGCTTTACCNAGACCACTTACCCATGCTCCTGCGAGAAG | | |
| | | GATCAAGGNAGAGGACNACCAGCTCATTGTGAAGAAAGGATTCTTGCGAGGCTGATAACAGCACTGTNGC | | |
| | BGH sequence | CATTATAAACCCTCCTTTAATAATTGATTCCAGAGATGAGNGNATGGAACCCCTCCCCCACCCTGCAAGGNACAGCCT | | |
| | | CACCCACCCTTAGCGCAGAGGACAGGGGACAGCTGCCAAGAAACACCAGTCCAGATCCTCCTCTCATCCAGGGTCTN | | |
| | | GNGCCAGACCTGAGGGACCCACACCCCTAAGTNGTCAGGTCCCTCACCAAGAGGAGCGCACCAGAGGCTACCTGGG | | |
| | | CCAGTCCCCCAGGAGGCCCCTCAGTTCAGTTCCCTGCTGAACTGAGCTNGGGGGGGGGGAG | | |
| | Identity | dbj\|D14883\|MUSC33R2IA Mouse mRNA for C33/R2/IA4, complete cds | | |
| | | Length = 1657 | | |
| | | Score = 91.7 bits (46), Expect = 3e−17 | | |
| | | Identities = 52/55 (94%), Positives = 52/55 (94%) | | |
| | | gb\|AF049882\| Rattus norvegicus metastasis suppressor homolog (KAI1) mRNA, | | |
| | | complete cds | | |
| | | Length = 1740 | | |
| | | Score = 69.9 bits (35), Expect = 1e−10 | | |
| | | Identities = 56/65 (86%), Positives = 56/65 (86%) | | |
| | Comments: | Data so far shown: | | |
| | | C33-induced apoptosis is mediated by the induction of free radicals of oxygen | | |
| | | which lead to proapoptotic activation of mitochondria. These free radicals of | | |
| | | oxygen are not generated by the mitochondrial respiratory chain, since cells | | |
| | | having a genetic defect in the respiratory chain still react to C33 expression | | |
| | | with apoptosis. The proapoptotic activity of C33 is independent of substrate | | |
| | | or cell-cell interactions, since even cells in susppension are driven into | | |
| | | apoptosis by C33. The rapid kinetics of apoptosis induction by C33, too, | | |
| | | differs from the proapoptotic effects of C33 stated so far. Moreover, we have | | |
| | | shown that an extracellular loop in C33 which seems to be responsible for | | |
| | | substrate interaction is not needed for the induction of apoptosis. | | |
| Clone # | T7 sequence | AAAGGAGCCCTNGAAAGCGACATGGCGGNTCTCTTAAAGCTGGGCGNTCTCTGCAGNGGNCAAGGAGCTCGAGCTC | | |
| 26 | | TCCTACTCCGAAGCCGGGTGGTCAGACCCGCTTATGTGTCAGCATTTCTCCAGGACCAGCCTACCCAAGGACGGAG | | |
| | | TGGTACCCAGCACATTCACCTGTCACCAAGCCACCACTCTGGTTTCAAGGCTGCATCTCTCCACTGGANCAGTGAGA | | |
| | | GGGAAGNNNGTGCCC | | |
| | Identity | gb\|U50987\|BTU50987 Bos taurus succinate-ubiquinone reductase membrane | | |
| | | anchor subunit precursor QPs3 mRNA, complete cds | | |
| | | Length = 1317 | | |
| | | Score = 127 bits (64), Expect = 1e−27 | | |
| | | Identities = 120/139 (86%), Positives = 120/139 (86%) | | |
| | | dbj\|AB006202\|AB006202 Homo sapiens mRNA for cytochrome b small subunit | | |
| | | of complex II, complete cds | | |
| | | Length = 1313 | | |
| | | Score = 79.8 bits (40), Expect = 2e−13 | | |
| | | Identities = 114/139 (82%), Positives = 114/139 (82%) | | |
| Clone # | T7 sequence | AAAGGCAGATCGAGAGGGGCATGNGGGCCAACGAACAGGCGCTGGCGTCTGGCCNGAGNGAGNGAGTTCTCATNA | | |
| 27 | | CTGGGGGCATNGAGGCTACNGCTGGACGTTTCACACAGAGGTATTTCGGCGCCTACTCTATCGCTGCAGGNGNGCT | | |
| | | CATCNGTCTGCTGGAGTATCCCCGGNGAAAGAGGAAAAAGGGGACCANCAT | | |
| | Identity | gb\|M31775\|MUSCYTB558 Mouse cytochrome beta-558 mRNA, 3′ end. | | |
| | | Length = 677 | | |
| | | Score = 240 bits (121), Expect = 8e−62 | | |
| | | Identities = 176/199 (88%), Positives = 176/199 (88%), Gaps = 9/199 (4%) | | |
| Clone # | T7 sequence | AAAGCGCGCAGACCGCTCCTCCGCTGCAGAGTCGNTTNCCNGAGCTNGGNCGACAAGGCNGCCTTCGCAGNCGGGA | | |
| 28 | | NCCTGCCAGCCGNGACCCCAGCCTTCG | | |
| | BGH sequence | AAGATAANGGTTTTTAATTGAGTTATNGAGATGAAGAGACAGNGAAGCCCTGTTNGCTACTTACATGAAAAGAAGAT | | |
| | | TTTAAAAAACAATCACTGCACAAAATACAAAGGGGCAGGGNANGCNGAGGCATNGAATTCCTCCCCACGNTTTTTCT | | |
| | | NGACTTCTCAAGAACAAATTAAAGTCTCCACAGCAAATTNGNTCTCAAAANGCCGAANGGNGAAACAGTTACNGGCT | | |
| | | TCCCGCTTCNGAATACCTCTAATNGTTNCCCGGCGCTGCAGCCNGTAGGNCTCCTTGNCGTGACACAGTCGNNAGAT | | |
| | | GAAGAAGCCCAGGTNGTCCACGTTCTCGANGCNGACGCCGATCACCATGTGCTCANGGATACG | | |
| | Identity | gbj\|U76253\|MMU76253 Mus musculus E25B protein mRNA, complete cds | | |
| | | Length = 1790 | | |
| | | Score = 117 bits (59), Expect = 4e−25 | | |
| | | Identities = 88/99 (88%), Positives = 88/99 (88%), Gaps = 1/99 (1%) | | |
| | Description | DNA ladder | | |
| Clone # | T7 sequence | AAAGAGCGGCTGCTGTCGGAAGCACCGGGCGAGCTATCTGTTACAGTCCGGCCCGGGGATGGCTCGGGACGCGGA | | |
| 29 | | GCTGGCGCGCAGTAGCGGGTGGCCGNGGCGGNGGCTGCCGGCGCTGCTGCTGCTGCAGCTGCTGCGGTGGAGGT | | |
| | | GCGCCCTGTGCGCGCTCCCCTTCACCAGCAGTCGGCACCCAGGCTTTGCGGACCTGCTGTCGGAGCAGCAGCTGTT | | |
| | | GGAGGTGCAGGACTTGACCCTGTCTTTGCTGCAGGGCGGAGGTCTAGGGCCGCTGTCACTGCTACCTCCGGACCT | | |
| | | GCCGGATCTGGAGCCTGAGTGCCGGGAGCTGCTGATGGACTTCGCCAATAGCAGCGCCGAGCTGACCGCCTGTAT | | |
| | | GGNGCGCAGCGCTCGGCCCGTGCGCCTCTGCCAGACCTGCTACCCGCTCTTCCAACAGGTCGCAATCAAGATGGAC | | |
| | | AACATCAGCCGAAACATCGGGAATACCTCCGAGGGCCCGCGCTGAGGCGGAAGTCTCCTGACGGCAGACAGAATGC | | |
| | | AGATAGTTCTCATGGNCTCTGAGTTTTTCAACAGCACGTGGCAGGAGGCGAACTGCGCAAATTGCCTAACAAACAAT | | |
| | | GGTGAGGATTTGTCAAACAACACAGAGGACTTCCTCAGTCTGTTTAACAAGACTTTGGCCTGCTTTGAGCATAACCT | | |
| | | GCAGGGGCACACATACAGNCTCCTTCCACCAAAAAATTACTCCGAAGTGTGCAGAAACTTGTAAAGAGGCATATAAA | | |
| | | AACCTGAGCCTNCTGTACAGTCAAATGNANAC | | |
| | BGH sequence | CCTTGTTATTTCCTTTATTGNAAAGCATAAGGAAAAAACAGGTTTTCTTGNGCACACACATAACCCTATGNGCCTAAG | | |
| | | GATTCAGAANTATGTNCATTTTTTTAATATGACCACAAGATGAAATTNTTTGGCACATTTTCAAATATATTTCTAATG | | |
| | | CAACCTNTAGAGAGCCAGACCCTGATCAGGAACAGAGANGGCTGGCTTGTTAAAGGGCTCTCCAGCTTNTTAGCCAA | | |
| | | AAGCAGNGGTTTGTNCACACAGTACTGAAAGGNACCCGAGGAGTCGCTACTCACAGTTTAAATATGTCACTTCACTC | | |
| | | ANTTTGCNCATGTAAATAAGNTTTACATGTACTGATGAAGATGGNTTCCAATGACCCTNAACCATGNGCTTCAAATCA | | |
| | | AGACAGGAACAATGACAGCNCAATGAACCCCGGCACATNTAGGGGATCACAGCGNCGNCTGATTGTCACATACCCGG | | |
| | | GGTGACACACTCTGGGACTAAGACTAGCCTGCTNTCACACTCTGCANATGTGGNAAACATACAAAAAATACCCAAAC | | |
| | | ACTCCTGCCTTCCTGTAGGGCAAANACAGGTTTTNAAGG | | |
| | Identity | no complete scores | | |
| | | >emb\|Z98200.8\|HS111B22 Human DNA sequence from clone RP1-111B22 on | | |
| | | chromosome 6q16-21. Contains a novel pseudogene, a pseudogene similar to | | |
| | | ribosomal protein L3, ESTs, STSs, GSSs and CpG Islands, complete sequence | | |
| | | [Homo sapiens ] | | |
| | | Length = 153381 | | |
| | | Score = 260 bits (131), Expect = 7e−67 | | |
| | | Identities = 267/312 (85%), Gaps = 3/312 (0%) | | |
| | | Strand = Plus/Plus | | |
| | | >gb\|AW489000.1\|AW489000 UI-M-BH3-asd-a-10-0-UI.s1 NIH_BMAP_M_S4 Mus musculus | | |
| | | cDNA clone UI-M-BH3-asd-a-10-0-UI 3′ | | |
| | | Length = 462 | | |
| | | Score = 438 bits (221), Expect = e−120 | | |
| | | Identities = 235/239 (98%), Gaps = 1/239 (0%) | | |
| | | Strand = Plus/Plus | | |
| | | >gb\|AW488401.1\|AW488401 UI-M-BH3-art-b-02-0-UI.s1 NIH_BMAP_M_S4 Mus musculus | | |
| | | cDNA clone UI-M-bH3-art-b-02-0-UI 3′ | | |
| | | Length = 497 | | |
| | | Score = 438 bits (221), Expect = e−120 | | |
| | | Identities = 235/239 (98%), Gaps = 1/239 (0%) | | |
| | | Strand = Plus/Plus | | |
| Clone # | T7 sequence | AAAGGCGCGGAGCCTGCTGGTGCCATGGNGGCTGGNGGCTGGGTACACTATGTCTAGAGTGGGGGCCCTGGGGGG | | |
| 30 | | CTCCCGCGCCGGGTTGGGACTGTTACTTGGTACTGCCGCCGGCCTTGGATTCCTGNGCGTCCTTTACAGCCAGCGA | | |
| | | TGGAAACGGACCCAGCGCCATGGCCGGAGTCACAGTCTGCCCAACTCCCTGGACTATGCGCAGGCTTCAGAGCGNG | | |
| | | GACGCCAGGTGACACAGTTTCGGGCTATCCCAGGAGAAGCTGGAGATGCTGCCATACTGCCCAGCCTCTCACAGGA | | |
| | | AGGGCAGGAGAAGGNGCTGGACCGCCTGGACTTTGAGCTGACCAGTCTTATGGCGCTGCGGCGCGAGGNGGAGGA | | |
| | | GCTTCAGAGAAGCCTGCAAGGACTAGCTGNCGAGAT | | |
| | BGH sequence | GACGGACTGGACGCCGCCTCCACATCCAGGTCCAGAGAGTCCTTCCTCCCCATCCTCACGGTCTCGCAGCTCACTTC | | |
| | | GTCCTCAGCATCTCCACTCTCCTTGTCGGAGTCCCGCTCGTAATCAGACTCCGCGTNGGCTGNNGTATAGCCTCCCT | | |
| | | CGCTCTCGGCGTCTGNGAGNGCGGCCCCTGAGGAGGCGGNGAAGTAGACAGAGCTGGAGCCCGNGGAGTCACTCC | | |
| | | TCTCTCTGGCAAAAGGGAACCTGCGCCGCCGGGCCACTCTCTGGTTCTCTTCTATATGAGAGCGGACCTCCCCGACA | | |
| | | ATCTCCCCAGCTAGTCCTTGCAGGCTTCTCTGAAGCTCCTCCACCTCGCGCCGNAGCGCCATAAGACTGGTCAGCAC | | |
| | | AAAGTCCAGGCGGTCCAGCACCTTCTCCTGCCCTTCCTGNGAGAGGCTGGGCAGTATGGCAGCATCTCCAGCTTCA | | |
| | | CCTGGGATAGCCCGAAACTGTGTCACCTGGCGNCCACGCTCTGAAGCCTGCGCATAGTCCAGGGAGNTGGGCAGAC | | |
| | | TGTGACTCCGGCCATGGCGCTGGGNCCGATTCCATCGCTGGCTGTAAAGGACGCACAGGAATCCAAGGCCGGNGGC | | |
| | | AGTACCAAGTAACAGTCCNAACCCGGCGCGGGAGCCGCCCAGGGCCCCCAGTCTAGACATAGTGTACCCAGCCACCA | | |
| | | GCCACCATGGCAGCAGCAGGCTTCGCGCCTTTGCAGCACACTGGCGGCCGGTACTAGTGGANCCGAGCTTNGGNAC | | |
| | | CAAGCTTG | | |
| | Identity | gb\|AI115883\|AI115883 ue96a12.y1 Sugano mouse embryo mewa Mus musculus cDNA | | |
| | | clone 1498942 5′, mRNA sequence [Mus musculus] | | |
| | | Length = 540 | | |
| | | Score = 731 bets (369), Expect = 0.0 | | |
| | | Identities = 393/403 (97%), Positives = 393/403 (97%) | | |
| | | gb\|AA052396\|AA052396 mb67c07.r1 Soares mouse p3NMF19.5 Mus musculus cDNA | | |
| | | clone 334476 5′ | | |
| | | Length = 405 | | |
| | | Score = 660 bits (333), Expect = 0.0 | | |
| | | Identities = 380/394 (96%), Positives = 380/394 (96%), Gaps = 4/394 (1%) | | |
| | | gb\|AA777720\|AA777720 z\|06a08.s1 Soares fetal liver spleen 1NFLS S1 Homo | | |
| | | sapiens cDNA clone 449462 3′ | | |
| | | Length = 453 | | |
| | | Score = 589 bits (297), Expect = e−166 | | |
| | | Identities = 398/435 (91%), Positives = 398/435 (91%), Gaps = 4/435 (0%) | | |
| | | gb\|AA960116\|AA960116 ub54e08.s1 Soares mouse mammary gland NMLMG Mus musculus cDNA clone 1381574 3′ | | |
| | | Length = 416 | | |
| | | Score = 543 bits (274), Expect = e−153 | | |
| | | Identities = 354/385 (91%), Positives = 354/385 (91%), Gaps = 2/385 (0%) | | |
| | Description | DNA ladder, | | |
| Clone # | T7 sequence | AAAGGCTTGCCNTNCAGGCCATGCGGCTGGAGGTNTGCATCGAGGCGTTTCCCATGTTTCTGCTGAACCTTCTAGG | | |
| 31 | | CATGNGGAGCTGGGTATGCAAAAAGNGCTTTCCCTACTTCCTGAAGCGGTTCGCCATGATATACAATNGGAAGATG | | |
| | | GCGAGCCTAAAGCGGGAGCTCTTCAGCAATCTGCAGGAGTTCGCCGGCCCCTNGGGGAAGCTAACTCGCTGGAGG | | |
| | | NGGGCTGCGGCACCGGGGCCAACTTCAAGTTCTATCCCCCCGGGAGCAGGGTCACTNGTATCGACCCTAANCCCAA | | |
| | | CTTTGAGAAGNTCTTGTTCAAGAGCGTCGCANAGAACCGGCAGCTGCAGTNCGAGCGCTTNGAGGNGGCAGCCGNN | | |
| | | GAGGACATGCACCAGGTGACCGANGGCTTCTGAGGACCGTG | | |
| | BGH sequence | GGCAAATATAGAAACCATTTATCAAANGAATATAAANGTATTGATCAAGATTTAAAATATAACTTCTGCAAAATCATN | | |
| | | TTGAAAAATATACATTTGTTTAGATCCATACATACAAATGCAGCTGAAACCCTTGGGCCCACCCAGACTTGCTCTCTG | | |
| | | TATGAACACAANGATATCCANGGTTTTGTTTCAGGACCAGNGGAATTTTTCTTCTTCTTCAATACAGGGTTNATTNG | | |
| | | NGNAGCCCTGGCGGNCCTGGAACTCCATCTGTAGATCAGGCTGTACTNACAGAGATCCACCTACCTCTGCCACCTGA | | |
| | | GAGCTGGGATTGAGGCTNTGCCACCAACCACTCAGGACCAGNGATATTTGACCAGAAGAATCCCTCCCCCCCCGNAC | | |
| | | CCCGAGTCCTTGGNAACTACTCTCGGGCATTACTTTTAGGGNCCCCTACATACTGNCCCATTTCCCTAACTATAGN | | |
| | | GGCCTTCTACTGCCTACGGNAATNATATTCAGCAAAGTNTGTGCTAGATAAGATGGNAAATTAAACAGAGAATTCCC | | |
| | | ATCTGNNCTGNGCNGNNCTGNGATGACGCT | | |
| | Identity | gb\|AI315450\|AI315450 uj46g05.y1 Sugano mouse liver mlia Mus musculus cDNA clone IMAGE:1923032 5′, mRNA sequence [Mus musculus] | | |
| | | Length = 469 | | |
| | | Score = 511 bits (258), Expect = e−143 | | |
| | | Identities = 363/404 (89%), Positives = 363/404 (89%) | | |
| | | gb\|A1019878\|AI019678 ua92d11.r1 Soares mouse mammary gland NbMMG Mus musculus cDNA clone 1364949 5′ | | |
| | | Length = 431 | | |
| | | Score = 505 bits (255), Expect = e−141 | | |
| | | Identities = 363/405 (89%), Positives = 363/405 (89%) | | |
| Clone # | T7 sequence | AAAGAAGGAACTAAACATGGGCCAGCGATGCTCTGACACCAGAGGAATNGCTTTCGAAGTCAGAGNGCCTAAG | | |
| 32 | | GAAAATGTGTTAATCGGNGAAGGAGCANTTTTCAAGATCGCAANGGGTGCTTTNGATAGAACCAGACCTACAGTCG | | |
| | | CAGCTGGCGCTGNCGGGCTAGCCCAGAGAGCTCTGGACGAAGCCACGAAGTATGCCCTGGATAGGAAGACATTTGG | | |
| | | AAAGCTGCTAGNGGAGCACCAAGGAGTTTCATTTCTGCTCGCAGAAATGGCNATGAAGGTNGAACTCGCTAGGCTC | | |
| | | AGTTACCAGAGAGCAGCCTGGGAGGNNGACTCCGGTCGGCGGAACACTTACTATGCCTCGATTGCAAAGGCCTTTG | | |
| | | CTGGAGACATTGCCAATCAGCTAGCCACTNACGCCGNGCAGATTTTCGGNGGCTANGGATTNAACACAGAGTACCCT | | |
| | | GTGGAGAAGCTNANGNNGNGCGCCANGATCTATCANATCTATNANGGTC | | |
| | Identity | gb\|U07159\|MMU07159 Mus musculus medium-chain acyl-CoA dehydrogenase mRNA, complete cds. | | |
| | | Length = 1846 | | |
| | | Score = 799 bits (403), Expect = 0.0 | | |
| | | Identities= 461/488_(94%), Positives = 461/488 (94%) | | |
| Clone # | T7 sequence | AAAGCGTCGCCATCCGCCACCATGGNGAACTTNANAGTAGATCAGATCCGNGCCATCATGGACAAGAAAGCCAACAT | | |
| 33 | | CGATAAGGGNGAGGNGTCTGCCCGCCAGGAGCTCAAGGCACGTGCCCGCTACCTGGCCGAAAAGTNTGAGNGGGA | | |
| | | CGTTGCTGAAGCCCGNAAGATCNGGNGCTTAGGCCCTGNTGGCACTGGCCCCAACATTCTNACCGACATNACCAAGG | | |
| | | GNGTGCAGTACCNGAATGNGATCAAGGACAGCGNGGAGGCNGGCTTCCAGNGGNCTNCTNAGGNGGGCGCTNTCTN | | |
| | | NGAGNANANCATGCNNCATTNTGCNCCC | | |
| | Identity | gb\|M76131\|MUSEF2 Mouse elongation factor 2 (ef-2) mRNA, 3′ end. | | |
| | | Length = 1179 | | |
| | | Score = 309 bits (156), Expect = 2e−82 | | |
| | | Identities = 207/231 (89%) | | |
| | | Strand = Plus/Plus | | |
| Clone # | T7 sequence | AAAGGCCAGCTCCTGCTCTGCTCCTCTCCCGCCTGCCGCCGCGCTGCACGCCTCGAGCACTCCCTCGGCCCCGGCG | | |
| 34 | | GNGACCGGGGACCCCGCAGCTACCGCCATGCTGCCAGNGCTCTACACCGGCCTGGCGGGGCTGCTGCTGCTGCCTC | | |
| | | TGCTGCTCACCTGCTGCTGCCCCTACCTCCTCCAAGATGNGCGGNACTTCCTGNGGCTGGCCAACATGGCCCGGCG | | |
| | | GGNGCGCAGCTACCGGCAGCGGCGACCCGNGCGTACCATCCTGCGGGCCTTCCTGGAACAAGCGCGCAAGACCCCA | | |
| | | CACAAGCCCTTCCTGCTGTTCCGAGACGAGACGCTCACCTACGCCCAGGAGGACCGGCGNAGCAANCAAGNGGCGC | | |
| | | GGGCGCTGCACGATCAACTGGGCCTACNANAGGGGGATTGCGTAGCCCTCTTNATGGGCAATGAGCCGGCCTACGN | | |
| | | GNGGATCTGGCTGCNACTGCTNAAACTGGGCNGCCCATGNCGAGCCTTANCTACAACATTCGTNCNAAGTCTCTGCT | | |
| | | GTACTGCTTTCAATGCTNCGGNGCCNAANGNGCAGNTTNCCTCCCAGNNNTACATGAAGCTNC | | |
| | Identity | gb\|AF072757\|AF072757 Mus musculus fatty acid transport protein 2 mRNA, complete cds | | |
| | | Length = 1872 | | |
| | | Score = 579 bits (292), Expect = e−163 | | |
| | | Identities = 356/383 (92%), Gaps = 1/383 (0%) | | |
| | | Strand = Plus/Plus | | |
| | | Score = 58.0 bits (29), Expect = 2e−06 | | |
| | | Identities = 31/32 (96%) | | |
| | | Strand = Plus/Plus | | |
| Clone # | T7 sequence | AAAGCCTCCACTTGCCTACTTGGGGCGCGAGGAGGTTGGAGAGTTTTTTTCTGGGACCCAAGCAAAGGCATCCACG | | |
| 35 | | CTGCTGCTAAGCTGAAATTGAAGCTCACACATCCTGGAAAATGCTAGCACCCATACCAGAACCCAAGCCTGGAGACC | | |
| | | TGATTGAGATTTTCCGCCCTATGTACAGACACTGGGCCATCTATGTTGGNGATGGATACGTGATCCACCTGGCTCCT | | |
| | | CCAAGTGAAATCGCAGGAGCTGGGGCAGCCAGCATCATGTCTGCTTTGACTGACAAGGCCATAGTGAAGAAAGAAC | | |
| | | TGCTGTGCCATGTGGCCGGGAAGGACAAGTACCAGGTCAATAACAAACATGACGAGGAGTACACCCCACTGCCTCT | | |
| | | GAGCAAGATCATCCAGCGGGCTGAGAGACTGGNGGGGCAGGAGGTGCTCTACAGGCTGACCAGCGAGAACTGTGA | | |
| | | GCACTTTGTGAATGAACTACGCTATGGAGTTCCTCGGAGTGATCAGGTCAGAGATGCGGNCAAGGCGGTAGGCATC | | |
| | | GCTGGAGTGGGCTTGGCGGCCTTGGGCCTCGTTGGAGTCATGCTCTCCAGAAACAAGAAACAGAAGCAATGAGCTG | | |
| | | AATGACTGCCCAGTTTTTGGGCTCTTCTTTTGCTAGAGGGTTTGGAGTTTGATTTATAGATTCTATTGCTTTATAAT | | |
| | | TAGGGTTATTTTCACAACATACANTAAACCACAAGAAAGGAA | | |
| | BGH sequence | CTNGAGGNTAATAGTATGNNGTGAAAATANANNTAATTATAAAGCANTAGAANTTATAAATCAAACTCCAAANCCTNT | | |
| | | AGCAAAAGAAGAGCCCAAAAACNGNGNAGNCATTCAGCTNATTGNTTCTGTCTNANNGNNTCNGGAGAGCANGACTT | | |
| | | CANTGNGNNCCAAGGANGNCAAGCNCACACNAGNGNNGACTACTCGACTAGCCCGCATT | | |
| | Identity | emb\|X76453\|RNHREV107 R. norvegicus (Sprague Dawley) H-rev107 mRNA | | |
| | | Length = 966 | | |
| | | Score = 745 bits (376), Expect = 0.0 | | |
| | | Identities = 546/603 (90%), Gaps = 1/603 (0%) | | |
| | | Strand = Plus/Plus | | |
| | | emb\|X92814\|HSHREV107 H. sapiens mRNA for rat HREV107-like protein | | |
| | | Length = 1070 | | |
| | | Score = 323 bits (163), Expect = 3e−86 | | |
| | | Identities = 334/389 (85%), Gaps = 2/389 (0%). | | |
| | | Strand = Plus/Plus | | |
| | Description | PI-FACS: Apoptosis | | |
| Clone # | BGH sequence | GGGGCACAGTCTATTTTATATAANGAGATCAGTTGTCTGGAAAGGGATATGGTGTCCAGAGTGAGATAGGGGACCC | | |
| 36 | | TGGGCTNGGGACCCATTGTTCAGATAGGAAAACTGAGATCAGGGCAGGTATGATTNGGGTTAGGGAGNCNGNGNG | | |
| | | GAGCGGGGGGTNAACCTCAGCAGGGCAGGCACTGNGACCCTGATCTTCTCAGANTTCAGGAAAGCCACGTGCTTCT | | |
| | | CATAATAGGCAGCCTCATTAATGAACACAGGGTACACAATGGAGTCCCCCTCATACAGTACGTCTTCTCCGCTGAAA | | |
| | | AGCTTGAAGATGGGTTCANCAGGNCTNAGNGGCTCAAAGTCATGGNCGTGCAGNTGAGGGNGCACAGAGNCAGCCA | | |
| | | GGTCACCATCCNCAGTGCGTGGGAAGTCCACACTGCCCAAGTTCCTGTANATATCCATCTCAAAGGCGGGTAAGTCC | | |
| | | ATGCCTTGGTTGAAGAGCTCGATGAAGTCCNNAATGGATGCCACCAGAGCTNGCATCCGNNAGAACAGGNCGGCCC | | |
| | | GCAGCACGCCCCTGAGGCTGCGGGCCCATCTCCNGACAGATTCCATTCTTGGNNATAGATTCCCCCTTTCCAGCACA | | |
| | | CTGNCGGCCNGNNACTAGT | | |
| | Identity | >gb\|AW107362.1\|AW107362 um15a04.x1 Sugano mouse kidney mkia Mus musculus | | |
| | | cDNA clone IMAGE:2192334 3′ similar to SW:ACY2_HUMAN P45381 ASPARTOACYLASE; | | |
| | | Length = 649 | | |
| | | Score = 930 bits (469), Expect = 0.0 | | |
| | | Identities = 557/595 (93%). Gaps 1/595 (0%) | | |
| | | Strand = Plus/Plus | | |
| Clone # | T7 sequence | AAAGCTGCATNGNGGCGTTACCCATG1TTCNGCTNAACGTTCTAGGCATGNGGAGCTGGGTATGCAAAAAGNGCTT | | |
| 37 | | TCCCTACTTCCNGAAGCGGNTCGCCANGATATACAATNGGAAGATGGCGAGCCTAAAGCGGNAGCTCTTCAGCAATC | | |
| | | TGCAGGAGNNCGCCGGNCCCTCGGGGAAGCTAANTCAGCTGNNGGAG | | |
| | BGH sequence | NGGCAAATATAGAAACNATTTATCAAATGAATATAAANGTATTGATCAACATTTAAAATATAANTTCTGCAAAATCAT | | |
| | | CTTGAAAAATATACATTNGTTTAGATCCATACATACAAATGCAGCTGAAACCCTTGGGCCCACCCAGACTTGCTCTCT | | |
| | | GTATGAANACAATGATATCCATGGNTTNGTTTCAGGACCAGNGGAATTTTNCTTCTTCTTCANTACAGGGTTTATTT | | |
| | | GTGTAGCCCTGGNGGCCCTGNAACNCNATTTGTAGATCAGNCTGT | | |
| | Identity | gb\|AI315920\|AI315920 uj27f11.y1 Sugano mouse kidney mkia Mus musculus cDNA clone IMAGE:1921197 5′ similar to TR:Q14521 Q14521 GIANT LARVAE HOMOLOGUE; mRNA sequence [Mus musculus] | | |
| | | Length = 689 | | |
| | | Score = 190 bits (96), Expect = 5e−47 | | |
| | | Identities = 144/164 (87%) | | |
| | | Strand = Plus/Plus | | |
| | | gb\|AI315072\|AI315072 uj23e11.x1 Sugano mouse kidney mkia Mus musculus cDNA clone IMAGE:1920812 3′. mRNA sequence [Mus musculus] | | |
| | | Length = 538 | | |
| | | Score = 385 bits (194), Expect = e−105 | | |
| | | Identities = 220/232 (94%) | | |
| | | Strand = Plus/Plus | | |
| | | gb\|AF109905\|MMHC213L3 Mus musculus major histocompatibility locus class III regions Hsc70t gene, partial cds; smRNP, G7A, NG23, MutS homolog, CLCP, NG24, NG25, and NG26 genes, complete cds; and unknown genes | | |
| | | Length =135545 | | |
| | | Score = 44.1 bits (22), Expect = 0.014 | | |
| | | Identities = 47/57 (82%) | | |
| | | Strand = Plus/Minus | | |
| | | Score = 42.1 bits (21), Expect = 0.054 | | |
| | | Identities = 24/25 (96%) | | |
| | | Strand = Plus/Plus | | |
| | | Score = 40.1 bits (20), Expect = 0.21 | | |
| | | Identities = 31/35 (88%) | | |
| | | Strand = Plus/Minus | | |
| | | Score = 36.2 bits (18), Expect = 3.3 | | |
| | | Identities = 46/57 (80%) | | |
| | | Strand = Plus/Plus | | |
| | | Score = 36.2 bits (18), Expect = 3.3 | | |
| | | Identities = 46/57 (80%) | | |
| | | Strand = Plus/Plus | | |
| | | gb\|AF110520\|MMHC425O18 Mus musculus major histocompatibility complex region NG27, NG28, RPS28, NADH oxidoreductase, NG29. KIFC1, Fas-binding protein, BING1, tapasin, RalGdS-like, KE2, BING4, beta 1,3-galactosyl transferase, and RPS18 genes, | | |
| | | Length = 213245 | | |
| | | Score = 44.1 bits (22), Expect = 0.014 | | |
| | | Identities = 47/57 (82%) | | |
| | | Strand = Plus/Plus | | |
| | | Score = 36.2 bits (18), Expect = 3.3 | | |
| | | Identities = 46/57 (80%) | | |
| | | Strand = Plus/Plus | | |
| Clone # | T7 sequence | AAAGGTGGAGCTGGGTGGTGTTTGGTGGGGTACGGCGGCCACTCAGTTGCAGCAGAGCAGGTGCCATCCTGTGGA | | |
| 38 | | AGAACCATGAAGCACTACGAGGTGGAGATTCGGGATGCAAAGACGAGGGAGAAGCTGTGCTTCCTGGACAAGGTA | | |
| | | GAGCCTCAGGCCACCATTTCTGAAATCAAGACCCTTTTCACCAAGACACACCCGCAGTGGTATCCTGCCCGCCAGTC | | |
| | | CCTCCGCCTGGACCCCAAGGGGAAGTCCCTGAAAGATGAAGATGTCTTACAGAAGCTTCCTGTGGGCACCACAGCC | | |
| | | ACACTCTACTTCCGGGACCTCGGGGCCCAGATCAGCTGGGTGACGGTCTTCCTGACGGAGTATGCCGGGCCCCTTT | | |
| | | TCATCTACCTGCTCTTCTACTTCCGGGTACCCTTCATTTATGGCCGCAAATACGACTTTACGTCCAGTCGGCATACG | | |
| | | GTGGTGCACCTCGCCTGCATGTGCCACTCGTTCCACTACATCAAGCGCCTGCTGGAGACTCTCTTCGTGCACCGATT | | |
| | | CTCTCACGGAACCATGCCTTTGCGAAACATCTTCAAAAACTGCACCTACTATTGGGGCTTTGCTGCATGGATGGCTT | | |
| | | ATTACATCAACCACCCTCTCTACACACCCCCTACCTATGGAGTTCAGCAGGTTAAGCTGGCACTGGCCGTTTTTGTG | | |
| | | ATCTGCCAGCTTGGGAACTTCTNCATCCACATGGCTCTTCGGGACCTTCGGCCTGCTGGGTCNAAAACCAGGAAGAT | | |
| | | CCATACCCCACCAAGAACCCCTTCACTGGCTGGTCCTGTGG | | |
| | BGH sequence | GGGTAAGCAGGTTTTATTGTTGCTGCTGGAGAGCCATGGCCAGGCACACATNAGGACAGGGGCAGNGAGGGGTGG | | |
| | | AGAGTATTACTGGGCAGAGCCGTGAGGAGCTGCTCAGAGCAGGAAGGGAATAATGGGCATGCGCAGGGGCGGGTA | | |
| | | GTCGCGGAACTCCTTCAGGTAGCTGCGGNGTTTGCCCTTGGCCCAGATAGTCATCTGGGTGAAGCCCACCAGGGAG | | |
| | | AAGAGGGCCACTGGGACACACTGAGTCAAGATGGCAAAGCCAATCCAGGAGCCCACCTNATAAGTGTAGTTGGGAC | | |
| | | AGGACACCAACAGGAACAGCCAGGTGAAGGGGTTCTTGGNGGGGTATGGGATCTTCCTGGTTTTCGACCCAGGAG | | |
| | | GCCGAAGGTCCCGAAGAGCCATGTGGATGGAGAAGTTCCCAAGCTGGCAGATCACAAAAACGGCCAGNGCCAGCTT | | |
| | | AACCTGCTGAACTCCATAGGTAGGGGGNGTGTAGAG | | |
| | Identity | gb\|S45663\|S45663 SC2 = synaptic glycoprotein [rats, brain, mRNA, 1178 nt] | | |
| | | Length = 1178 | | |
| | | Score = 650 bits (328), Expect = 0.0 | | |
| | | Identities = 450/490 (91%), Gaps = 6/490 (1%) | | |
| Clone # | T7 sequence | AAAGCGCTGGAGCTACAGCCGTTACTGCCGCCGCCGCCGCCGCCGGCGCCGAGGCGTTNGATCGTTGGCAATGTCA | | |
| 39 | | GGCTTTGATAACTTAAACAGCGGTTTCTACCAGACGAGTTACAGCATCGACGAGCAATCTNAGCAGTCCTATGACTA | | |
| | | TGGAGGAAGNGGAGGACCCTACAGCAAGNAGTATGCTGGCTGAGACTACTCGCAGCAAGGCCGATTNGTCCCTCCA | | |
| | | GACATGATGCAGCCACAGNAGACATACACTGGGCAGATTTACCAGCCAACTCAGGCCTATCCTCCAACAACANCTNA | | |
| | | GCCATTCTATGGAGACAGCTTNGAGGAGGAGCCCCCTCTGTTAGAAGAGTTGGGTATCAATTTTGACCACATTNGG | | |
| | | CAAAAAACACTAACGGAGCTACACCCCCCTGAGGGCAGNTGACGGCAGCATCATGAATGATNACGGACTTGNGCAG | | |
| | | GNCCCAG | | |
| | Identity | gb\|AA763399\|AA763399 vw53h02.r1 Soares mouse mammary gland NMLMG Mus musculus cDNA clone 1247571 5′ similar to WP:F32D8.4 CE05783 LACTATE DEHYDROGENASE; | | |
| | | Length = 635 | | |
| | | Score = 668 bits (337), Expect = 0.0 | | |
| | | Identities = 370/383 (96%), Gaps = 1/383 (0%) | | |
| | | Strand = Plus/Plus | | |
| Clone # | T7 sequence | AAATGGCCCTATGATGCAGAACTCTCCTTTNTCCTGCGCACGGGTACACGGTACGGAGNGGACACTNACCTGTTCAG | | |
| 40 | | NGNGGAGTCGCCGCAAGAGCTGGCAGCCTGGACCCGACAGTNGGAGGATGGCTGNCATCGGGCTGCTGAAGGCGT | | |
| | | ACAAGAAGNGTCTACAGCCTGCACGNGGAACGGCCGNCCCTGCAGCCTGTCTGNGCACATCGACAAGGGCTTCACC | | |
| | | CTGNGGGCAGCTGAGCCTGGAGCAGCCCGAGCCATGCTGCTCCGACAGCCCTTCGAGAAACTTCAGATGTCATCAG | | |
| | | ATGATGGCACGAGTCTCCTTTTCCTGGACTTNGGGGGNGCTGAAGGAGAGATCGAGCTGGACCTGCACTCGAGTCC | | |
| | | CAAAACGATGGTCCTNNATNATCCACTCTTTCCTGTCCNCNAAGGNCACCCNCTNGNCGCNCANGGCCTAGANGCCT | | |
| | | GNCCCGNTGNACCAGNNCCTTGAAAGCAGGCAATCTGATNCATCCTCG | | |
| | BGH sequence | GNGCTNANANGNTGNTNATTATTNAANANGAANGANGGGANANAGGACNAGGANAAAACCCAAATGCCCCACGGNGN | | |
| | | TTAAGGGGAAGNGAACNNAAAGGNTNCTCTTCTTNCTCTTGCCACTGACCCANAANANTCCANNTGNANANATCTNAN | | |
| | | GGNGGNNAANGGGCCTCNNNNAGGCCTGGCANNTNTGCTGNNGGNTGGGGACGGGATCCNCANAGGGNNANGNCCC | | |
| | | ANGCTNGNAAAGGTTTCTGNGTACNCNAAAAATNTNTTTTAAAAAGGCNCNGGNGGANGNNANCNNTANGAANGCTN | | |
| | | NCCAACNCAANCCNNACTTGCTNNCCCAGGNCTGAGNTNTGNTTCCTNAGGCTGNNCTCTGAGGCCC | | |
| | Identity | gb\|U00677\|U00677 Mus musculus syntrophin-1 gene, complete cds | | |
| | | Length = 2109 | | |
| | | Score = 626 bits (316), Expect = e−178 | | |
| | | Identities = 393/426 (92%), Gaps = 1/426 (0%) | | |
| | | Strand = Plus/Plus | | |
| | Description | DNA ladder | | |
| Clone # | T7 sequence | AGGCTGTGCGAAGCGGGCACGCGCGGACCCTTNACCGGCCGCGGAGCCGCTATGGGCCCGCCAGGCCCCTGGCGT | | |
| 41 | | GGCGCGGCGCAAGGACCGGCTCCNAGCCAGGATTGAAATTCCAAGATGGATGATCAGGACCCTGGGGGCATTAGCC | | |
| | | CCCTTCAGCAAATGGTGGCCTCAGGAGCCGGGGCTGNGGACACCTCCCTCTTCATGACACCCCTGNATGNGGTGAA | | |
| | | GGTCCGCCTTCAGTCTCAGAGACCCTCGGCAACCAGCGAATTGACAACTCCCTCCAGATTCTGGAGTCTCTCCTACA | | |
| | | CCAAATCATCCTCCGCTCTACAGTCCCCAGGGAAGTGCCTCCTATACTGNAATGGAGTCCTGGAGCCCCTGTACCTG | | |
| | | TGCCCAAATGGTACCCGNTGTGCCACCTGNTTTCAGGACCCCACACGGTTCACTGGCACCTTGGATGCCTTNGNGAA | | |
| | | GATTGNGCGGCATGAGGGCAGTAGGACCCTGTNNANNGGCCTCCNAGCCACCCTGGNGATGACCGNGCCAGCTACT | | |
| | | GCTATCTACTTCACTGCTTACGACCAACTCAAGGCCTTCCTGTGTGGCAGTCCTTGACCTCTGACCTCTACGCACCC | | |
| | | ATGGTGGCTGNTGCCCTCGCCCGAATGGGCACCGTGACAGTTGCAGCCCCTTGNAGCTCGTGCGGACCAAGCTGCA | | |
| | | GGCTCAGCATGTGTCATACCGTGAGCTGGCTTNCTCTGTTAAGCTGCNGNGACTCAGGGTGCTGGCGCTCT | | |
| | BGH sequence | NNNCNTTTNNCNCACAGAAGACCATCATNTTTAGACAAGGAATGAATGGNGCCAAGATACTGCCTGGCCCAAGNTCC | | |
| | | TCGGNCCAAGCTATAACAAACATGNGGATNCAAAAAAGGGCGATCATCAGATTTGNTCTTGAATTCGGNAGGCGGNA | | |
| | | GAGGGGCAATCCTGGNAAGGGAAACAGCAGGAATCCAGGTCTGAGGACAGCCTGGGAGACNGGACTGGNAGGNAA | | |
| | | GGCACTTGNCTCAGTCTCGTGATCCCACCCGTGCAAGAGCCGNNNGCTCCCCGACTTTCAGCGGCCCAGAGGCTGCT | | |
| | | NCTGGTNGAGCCTCTGNAAGAAGCTTTTGCCAAACTCNNAAGTGCTGANCATGATGGCCAG | | |
| | Identity | gb\|AC003043\|AC003043 Homo sapiens chromosome 17, clone HRPC1067M6, complete sequence [Homo sapiens] | | |
| | | Length = 139488 | | |
| | | Score = 133 bits (67), Expect = 5e−29 | | |
| | | Identities 112/128 (87%) | | |
| | | Strand = Plus/Plus | | |
| | | gb\|AA109006\|AA109006 ml63d04.r1 Stratagene mouse testis (#937308) Mus musculus cDNA clone 516679 5′ similar to WP:C16C10.1 CE01489 CARRIER PROTEIN; | | |
| | | Length = 517 | | |
| | | Score = 860 bits (434), Expect = 0.0 | | |
| | | Identities = 493/516 (95%), Gaps = 3/516 (0%) | | |
| | | Strand = Plus | | |
| | | gb\|AA985996\|AA985996 uc73c10.x1 Sugano mouse liver mlia Mus musculus cDNA clone 1431282 3′ similar to TR:O14589 O14589 SIMILARITY TO Q09461 | | |
| | | Length = 711 | | |
| | | Score = 553 bits (279), Expect = e−156 | | |
| | | Identities = 328/352 (93%) | | |
| | | Strand = Plus/Plus | | |
| Clone # | T7 sequence | AAAGCTTCACTTCGGCCTCCAGCCGGGGAGNCTGGAGCGCAACTTCCAGATAGCGGAGNGGCCTCAGCTGCGAGCC | | |
| 42 | | GAGCGGAGGCGGCAGCGTTNCTCAGGACACCCGCAGATCACCTTTTCCCCGCGACTTCGCCATGGCTGAGNGGTGA | | |
| | | GTACCGGTATGCCCACGGCCGATGNGTATCCCTCCACCCTATGCTGACCTCGGCAAAGCTGCCAGAGACATTTTCAA | | |
| | | CAAAGGATTNGGCTTAGGGCTGNNGAAGCTGGATGNGAAGACGAAGTCATGCAGCGGNGTGGAATTTTCAACATCT | | |
| | | GGCTCATCTAATACAGACACTGGTAAAGTTAGCGGGACCTNGGAGACCAAGTACAAATGGNGAGAGTATGGTCTGA | | |
| | | CTTTCACAGAGAAGTGGAACACCGANAACACTCTGGGGACAGAGATTGCANTTTGAAGACCAGATTTGTCAAGGATA | | |
| | | GAAACTTGACTTTNGACACCCAGCTTTTCACCGAACACAGGNNNAAGGANANNGTGGTNAATAATCAAGTCCTGCTT | | |
| | | TACCAAGNAGGGNGNAGNTGTC | | |
| | BGH sequence | CGGTTTTCATAAACGTCTATTTCATCATTGGTGGGTAGCACATTTAACAGTTAAATAGATTTAAATAATGTATAGGA | | |
| | | GGCCGNACCACGGCAGCACTGATAACCATCCAACTAGGAACCAGCCAACAGTGACTGTCTAAATATTTAAAATACAG | | |
| | | CTCTNGTTCATCATCCTTTGATGTGATCACCTTCTGGGGGAAGGAAGGGGAGCCTGCTGGTCGCATGGAAATATA | | |
| | | TTAAGGCCAAATCTTCTGATATTCGTTCCCAGAGGTTTCTTTTAACTGGATTAAGCCTCCAATTCCAAGGCAAGCCC | | |
| | | AAGTTTGNGGCCTCCAGCATTAAAGCTCTTCCCGTCTACCAGAGCAGACAGTGTAAGCTTCACACCAGGCCTCAGAG | | |
| | | TCTGAGTATAGCCCACTCCAATTAAACTAGAGTTGTTGACCTTTGCAGAGATAGAAGCAGTAGGATCCAACTGGTAT | | |
| | | TTAGCTGCAATGCCAAAACGAGTGCAGCNGGNACCTGATGTCCAAGCGAGGNTTACTGANGTGTCAAAATCTTNACA | | |
| | | TACTTTCTGATNAATTTGATCCTCCAAATTCTGTCCCATTATTTACATTTGTGTGNAGCTGGAAGTCCCCAGTCCTGT | | |
| | | AGCCGACNGCANAGNTACTC | | |
| | Identity | gb\|U30838\|MMU30838 Mus musculus voltage dependent anion channel 2 mRNA, nuclear gene encoding mitochondrial protein, complete cds | | |
| | | Length = 1662 | | |
| | | Score = 763 bits (385), Expect = 0.0 | | |
| | | Identities = 466/494 (94%), Gaps = 3/494 (0%) | | |
| | | Strand = Plus/Plus | | |
| | | gb\|L08666\|HUMPORlN Homo sapiens porin (por) mRNA, complete cds and truncated cds. | | |
| | | Length = 1464 | | |
| | | Score = 280 bits (141), Expect = 3e−73 | | |
| | | Identities = 252/291 (86%), Gaps 1/291 (0%) | | |
| | | Strand = Plus/Plus | | |
| | Description | DNA ladder | | |
| Clone # | T7 sequence | AAAGGGAGGAGGAAGCCCGGAGCGGAGCGGGGCNTCTGGGGGGGGTGGACCCGCCGCGGCTGCTGCTGCCACCG | | |
| 43 | | CCGCCGCCGCCACCACCGCTCGTGGGGCTCGTGGCGTGAGGAAGGAGGACGAGTGAGACCCCGGGGCGAGCGGGC | | |
| | | GGCGGCGCCGCTGCTGCTGCTGCTGCTGCGGGAGGGTCGGCGGCGGGACGGCGATGGCGGATATCGACAAACTCA | | |
| | | ACATCGACAGCATCATCCAACGGCTGCTGGAAGTGAGAGGGTCCAAGCCAGGCAAGAATGTCCAGCTCCAGGAGAA | | |
| | | CGAGATCCGAGGACTCTGCCTGAAGTCTCGGGAGATCTTCCTCAGTCAGCCTATCCTTTTAGAACTTGAAGCACCAC | | |
| | | TCAAGATATGTGGNGACATCCAGGGGCAGTACTATGATTTGCTCCGTCTGTTTGAATACGGNGGCTTTCCTCCAGA | | |
| | | GAGCAACTATTTGTTTCTCGGGGACTATGTGGACAGGGGCAAGCAGTCCCTGGAGACAATCTGCCTCTTGCTGGCC | | |
| | | TACAAAATCAAGTATCCGGAGAACTTCTTTCTTCTCAGAGGGAACCACGAGTGCGCCAGCATCAATAGGATCTACGG | | |
| | | ATTTTATGATGAGTGTAAAAGAAGATACAACATTAAGCTGTGGAAAACGTTCACAGACTGTTTTAACTGCTTGCCGA | | |
| | | TAGCAGCCATCGTGNACNAGAAGATATTCTGCTGCATGGAGGGTTATCACCAGATCTTCAATCTATGGAGCAGAATC | | |
| | | GGCCGAAATATGAGACCAACTGATNTACCAGATCAAGGNCTTCTTTTG | | |
| | BGH sequence | AAGTTAACAAGCTTGCATTTAATAAGTCTGAAACCATTCTCAGCACATGGCATTGTACACGGGCATCTGTGCAAACA | | |
| | | GATTCATTTAACAGGTCGNAGTTTAAAAAAGTCATAGATACTGNGAGTTCTGTATAAACCGGNGGACGGNAAGTTAG | | |
| | | TTCCTTTNGATTTATAAGCCTCAATGTCACCGNAGAATAAAGAATGTAGCCAAAGAAAGCATTATCGGTCACTCGTA | | |
| | | TAGGACAGAGTTGTTTCTATAATTTGAAGCTTTCTGAATGGACGGNTTCAGGCCTGATCCAACTGTAAAAAGATCAC | | |
| | | TCAGTGAATAGACTATATGGGAACTGTACAAAGTGTCATTAACTTNCATCATTAATAGCTTACTCAGCACTATACCAC | | |
| | | TATTGCTAGTTAAAATAACCTGCTTCTGAGGCCGCACGGAGGGAGGCGGCCTGTGCACGCAGCCTCGATGCCCTGG | | |
| | | CCACCTCATCCCCAGGGCGTGCCATACAGTCCAACAGAAACTTTGGCTTTAGGAAGGAATCACAGACNTTGAAAAGA | | |
| | | ATGGCTTTAATCATTATTAAATGTGCAGNGGGAAGGAGTGTGCTTCAGATAGTCTGGGCAGGGCTGGCGGCAGGC | | |
| | | AGGTCACTCCTGCTGGACAGCTGCAGACACTAGTTTGTCATGACAAGACAATGAGGGAAAGCAGNC | | |
| | Identity | gb\|M27071\|MUSDIS2M1A Mus musculus protein phosphatase type 1 (dis2m1) mRNA, complete cds. | | |
| | | Length = 1438 | | |
| | | Score = 1156 bits (583), Expect = 0.0 | | |
| | | Identities = 618/629 (98%), Gaps = 2/629 (0%) | | |
| | | Strand = Plus/Plus | | |
| | | Score = 232 bits (117), Expect = 9e−59 | | |
| | | Identities = 142/153 (92%) | | |
| | | Strand = Pius/Plus | | |
| | | gb\|U53456\|MMU53456 Mus musculus protein phosphatase 1cgamma (PP1cgamma) mRNA, complete cds | | |
| | | Length = 2294 | | |
| | | Score = 1156 bits (583), Expect = 0.0 | | |
| | | Identities = 618/629 (98%), Gaps = 2/629 (0%) | | |
| | | Strand = Plus/Plus | | |
| | | Score = 222 bits (112), Expect = 8e−56 | | |
| | | Identities = 112/112 (100%) | | |
| | | Strand Plus/Plus | | |
| | | Score = 52.0 bits (26), Expect = 2e−04 | | |
| | | Identities = 26/26 (100%) | | |
| | | Strand = Pius/Plus | | |
| | | emb\|X56438\|DMPP1A1 D.melanogaster PP1-alpha 96A gene for protein phosphatase 1 | | |
| | | Length = 2230 | | |
| | | Score = 145 bits (73), Expect = 2e−32 | | |
| | | Identities = 238/294 (80%) | | |
| | | Strand = Plus/Plus | | |
| | | gb\|M27067\|EMEBIMG Aspergillus nidulans phosphoprotein phosphatase 1 mRNA, complete cds. | | |
| | | Length = 2248 | | |
| | | Score = 85.7 bits (43), Expect = 1e−14 | | |
| | | Identities = 166/207 (80%) | | |
| | | Strand Plus/Plus | | |
| | | gb\|M60215\|MZEZMPP1 Z.mays protein phosphatase-1 (ZmPP1) mRNA, complete cds. | | |
| | | Length = 1644 | | |
| | | Score = 83.8 bits (42), Expect = 5e−14 | | |
| | | Identities = 122/146 (83%), Gaps = 2/146 (1%) | | |
| | | Strand = Plus/Plus | | |
| | | gb\|U00063\|CELF56C9 Ceenorhabditis elegans cosmid F56C9 | | |
| | | Length = 35028 | | |
| | | Score = 58.0 bits (29), Expect = 3e−06 | | |
| | | Identities = 103/127 (81%), Gaps = 1/127(0%) | | |
| | | Strand = Plus/Plus | | |
| | | Score = 44.1 bits (22), Expect = 0.042 | | |
| | | Identities = 43/50 (86%) | | |
| | | Strand = Plus/Plus | | |
| Clone # | T7 sequence | AAAGAAGAAGAGGGGGCTAAGCTGAGTATAGAGGTGCTCCAGACCAGCCTGCAGAAGGAACTGACTCTAAACAAAG | | |
| 44 | | GCCAGGCCTCCGCCATGGAGCTGCTGCGCTGCCCCACGCTTCGGCGCCTCTTCCTCTGCCTCTCTATGCTGNGGTT | | |
| | | TGCCACTAGCTTTGCCTACTACGGGCTGGTCATGGACCTGCAGGGCTTTGGGGTCAGCATGTACCTTATCCAGGTG | | |
| | | ATTTTCGGCGCTGNGGACCTGCCTGCCAAGTTTGNGTGCTTCCTAGTCATCAATTCCATGGGCCGCCGGCCTGCAC | | |
| | | AGTTGGCCTCCCTGCTGCTGGCAGGCATCTGTATCCTAGTGAATGGCATAATAGGGAGGGGCCATACAATCATTCG | | |
| | | CACATCCCTGGCTGTACTAGGGAAAGGCTGTCTGGCTTCCTCTTTCAACTGCATCTTCCTGTACACCGGAGAGCTGT | | |
| | | ACCCCACAATGATTCGGCAGACGGGCCTGGGCATGGGCAGCACCATGGCCCGGGTGGGCAGCATAGTGAGCCCACT | | |
| | | GATAAGCATGACTGCCGAGTTCTACCCCTCCATACCTCTCTTCATCTTCGGCGCTGNCCCCGTGGCCGCCAGCGCTG | | |
| | | TCACTGCCCTGCTGCCAGAGACCTTGGGCCAGCCGCTGCCTGATACAGTGCAGGACCTGAAGAGCAGGAGCAGAGG | | |
| | | AAAGCAGAAGCAACAGCAGCTGGAACAGCANAAGCAGATGATACCACTCCAGGTCTCAACACAAGAGAAGAACNGAC | | |
| | | TCTGAAAATGGAGAGGCGTCAC | | |
| | BGH sequence | CAAGGTAGAAGAAATTTATTTAATTGTCTGGGATTCTTTGCAATGTCCTGGAGGNGGAAGGGACAGGAGCTGGAGG | | |
| | | AGTGACCACTGAGCTGGAAGATGGCTGAGGAAGAGCTCATTCTGCTTAAGAAGCTGCACACAGTTAGAGCTTTNGT | | |
| | | TCCTAGTAGGTCTATTGAAGTGACCTTTGGGGAGGCATTTCTCTGAATGGCAGGCTCCGCATTTAGATGGCCCAGT | | |
| | | CCCTCCACTCACTCCCCCTCCTCATAGATGGNGGGACCTGCAGAACCCCACTCCCTTTAGNGCTGAGNGACGCCCTC | | |
| | | TCCATTTTCAGAGTCCGTTCTTCTCTTGAGTTGAGACCTGGAGNGGNATCATCTGCTTGTGCTGNTCCAGCTGCTGA | | |
| | | NGCTTCTGCTTTCCTCTGCTCCTGCTCTTCAGGTCCTGCACTGTATCAGGCAGCGGCTGGCCCAAGGTCTCTGGCA | | |
| | | GCAGGGCAGTGACAGCGCTGGCGGCCACGGGGACAGCGCCGAAGATGAAGAGAGGTATGGAGGGGTANGAACTCG | | |
| | | GCAGTCATGCTTATCAGTGGGCTCACTATGCTGCCCACCCGGGCCATGGNGCTGCCCATGCCCAGGCCCGNCTGCC | | |
| | | GAATCATTGNG | | |
| | Identity | gb\|U52842\|MMU52842 Mus musculus kidney-specific transport protein mRNA, complete cds | | |
| | | Length = 2161 | | |
| | | Score = 1501 bits (757). Expect = 0.0 | | |
| | | Identities = 769/775 (99%) | | |
| | | Strand = Plus/Plus | | |
| | | dbj\|AB004559\|AB004559 Rattus norvegicus mRNA for multispecific organic anion transporter, complete cds | | |
| | | Length = 2221 | | |
| | | Score = 1124 bits (567). Expect = 0.0 | | |
| | | Identities = 706/754 (93%) | | |
| | | Strand = Plus/Plus | | |
| Clone # | T7 sequence | AAAGACGNTACCCTGGAGTTCACCAGCATCGACGCTCACAAAGGCGTGGCCCCATCAAGACGNGGNGATTNGGAAA | | |
| 45 | | TACTNGGTTATNGCATGATCCAGNGGCTCAGCGGCTGTCTTCCTNGGGAAGATAAGTTGAAAGATCCTAACTACGTT | | |
| | | AGGGATTCCAAAATTAGATACAGAGACAACTGTGGCAGCTTTGANGGAGAAATGCTTTCCTGAGAAAAATAAGCCAG | | |
| | | GTGAGATCGCTAAGTACATGGAGTCTGNGAAACTACTGGAATACACCGAAAAACCTCTCTATCAAAACCTACGTGAT | | |
| | | ATCCTTTTACAAGGACTAAAAGCTNTAGGAAGTNAAGACTNACGGCAAACTGGNTTTTAGNGCTGAGGANAACGGAA | | |
| | | GTGNGAANCNAAGACCAGCCTCAAAGNNGCTTTAANAAANAANCANAANAAAGCNCNGNCGAGCGCNTGTNCNAGTG | | |
| | | ACATNTGAGNGCTCTANTGCACNCANCGTT | | |
| | BGH sequence | ATGAACCTGAGATTTATTTTTTCTTGTCAAAGTAACGAGNCTCTTATATGGAAAGCGGCTGTATATCTCTGAAGGAG | | |
| | | CAGTTAGAGAGCTGCTTCTGAATTCACCCAGAATTGCTACTGACCCTGAGCCCACATTTCCTTTCAGCTCTCCAGTC | | |
| | | TCGGGTTNGGCCAGAGGAGAGTCCACTCATGTGAAACATTCACTCCCTGACATTCCCATTCTAGGGTCTNAAAGAAC | | |
| | | GGNGGAGGAGCTGGAAGCAAGGGCAAGAGGAGACTCCAGTAGGTTNGGGCTTCTACTATAAATTCTCTTCTGAGAG | | |
| | | CACAGNAGAGGGGNCTGCGTGTCTGCGNCACGGNGAGGCTCACGCTTTATCTGCTGGAACTTGCACTGNGTCCAGC | | |
| | | CATAAAAGCGACAGTNGAGCAGCCCCTGGGATGCTGCTGAGAGAGCCTGGAGCACAGAGAGGGCCATGTGCAGGAA | | |
| | | GGNCTCCTGAGGCTCTGTCAGCTTCATGATCAGCAGGGAGACAGCTGNACCCCAGCAGCACACAAAGGCCACTGNN | | |
| | | AAGAAACGCACTCGCTGGCCCAGAGTGTGAATCACTGCCCACTGCTNACTCTCCAGAAGGCCCGNCGACTTTACANA | | |
| | | CTTCTTATATNACATC | | |
| | Identity | gb\|AF080252\|AF080252 Mus musculus serine/threonine protein kinase 51PK(S) mRNA, complete cds | | |
| | | Length = 1651 | | |
| | | Score = 640 bits (323), Expect = 0.0 | | |
| | | Identities = 379/401 (94%), Gaps = 2/401 (0%) | | |
| | | Strand = Plus/Plus | | |
| | | gb\|AF080253\|AF080253 Mus musculus serine/threonine protein kinase 51PK(L) mRNA, comptete cds | | |
| | | Length = 1743 | | |
| | | Score = 640 bits (323), Expect = 0.0 | | |
| | | Identities = 379/401 (94%), Gaps = 2/401 (0%) | | |
| | | Strand = Plus/Plus | | |
| | | dbj\|AB000449\|AB000449 Homo sapiens mRNA for VRK1, complete cds | | |
| | | Length = 1662 | | |
| | | Score = 222 bits (112), Expect = 5e−56 | | |
| | | identities = 268/322 (83%), Gaps = 1/322 (0%) | | |
| | | Strand = Plus/Plus | | |
| | Description | DNA ladder, PI-FACS: Apoptosis | | |
| Clone # | T7 sequence | AAAGCTGCGGCAGGCATTCTCGGAGGAAANNAGCCAAGGACTAACTACGANCATGAGATNGGCAGTGATTNGCTTT | | |
| 46 | | NGCCTATTTGGCATTGCCTCCTCCCTCGCGGNGAAAGTGACTGATTCTGGCAGCTCAGAGGAGAAGAAGCTTTACA | | |
| | | GCCTGCACCCAGATCCTATAGCCACATGGCTGGNGCCTGACCCATCTCAGAAGCAGAATCTCCTTGCGCCACAGAAT | | |
| | | GCTGNGTCCTCTGAAGAAAAGGATGACTTTAAGCAAGAAACTCTTCCAAGCAATTCNAATGAAAGCCATGACCACAT | | |
| | | GGACGACGATGATGACGATGATGATGACGATGGAGACCATGCAGAGAACGAGGATTCTGNGGACTCGGATGAATCT | | |
| | | GACGAATCTCACCATTCCGATGAGTCTGNTGAGACCTTCACTGCTAGTACACAAGCAGACACTTTCACTCCAATCGT | | |
| | | CCCCTNCAGTNCGATGTCCCCCCGACGGCCTGAGGNTGATAGCTTNGGCTTATGGGNCTGANNGTCCCAAGNTCTA | | |
| | | GNNAGTTTTCCCAGGNTTTCCTGNATGANACCANGCNATCCCNTGGAATNCCCACCNTATTGNAGGNNAACCCTTAAC | | |
| | | CCNTTCTTT | | |
| | BGH sequence | ACATGTACATGATTTTGGAATAATTTAATACTTTAACCTCAAGATACAACTATATTCTAAGACCATTATTTTAAAGGA | | |
| | | ACGGATCCTTACAAGACCAAAATAACCCATATAGCACGAGGTTGGTTTAGCCTTTCTTCTTCTTTCAACAAACGTGCA | | |
| | | CNACATGTTTCAGTAGCAAGGCCGATGCCATGGATATGAGAGCTGNGATTTGCAGGGACCAACCACATNTAGAACC | | |
| | | GGGGAGGCCAATCAGACGGNGGGTNGGGNGGCATTCTACGTAAATCAGCAGGTGACATNACAACACGCTGGGTGCA | | |
| | | GCCTCGCAACTGTCCATTAGGTTTCCTTTTTCTTGATGATCAGAGGNCCTACGTTGNCGCCAGTCTCTTCGTTGNGC | | |
| | | TTTATGTGAGCCCCATCGCAGAAGGGGAACTTTTTAGACCTCCAGCATNGGCAGTACACGGCCTTATCCCCCAGATC | | |
| | | CTCCATGTCGAAGGCATGCACCACCTTCGGNTTGACTTTCTGGATCTGAAGATTCACCATAGCTTTGGAGCGATTCT | | |
| | | CTTTAGCGTAGAACTTCTTGNAAGCCAGGNAACCGAGAGCGGCTGTGCCAGCAGCAAAGGTGACGGGCCGCGACCA | | |
| | | CTCAACTTNCACAGCGGAGNTTGNNGCTGAGGCCCATG | | |
| | Identity | gb\|J04806\|MUSOSP Mus musculus osteopontin mRNA, complete cds. | | |
| | | Length = 1385 | | |
| | | Score = 769 bits (388). Expect = 0.0 | | |
| | | Identities = 438/458 (95%), Gaps = 3/458 (0%) | | |
| | | Strand = Plus/Plus | | |
| | | emb\|X13986\|MMPONTIN Mouse mRNA for minopontin | | |
| | | Length = 1328 | | |
| | | Score = 724 bets (365), Expect = 0.0 | | |
| | | Identities = 408/425 (96%), Gaps = 3/425 (0%) | | |
| | | Strand = Plus/Plus | | |
| | | emb\|X1G151\|MMETA1 Mouse mRNA for early T-lymphocyte activation 1 protein (ETa-1) | | |
| | | Length = 1535 | | |
| | | Score = 718 bits 362 , Expect = 0.0 | | |
| | | Identities = 408/426 (95%), Gaps = 3/426 (0%) | | |
| | | Strand Plus/Plus | | |
| | | gb\|S78177\|S78177 Eta-1/Op (Eta-1b) = early T-lymphocyte activator-1 [mice, C3H/HeJ, spleen, mRNA, 1087 nt] | | |
| | | Length = 1087 | | |
| | | Score = 682 bits (344), Expect = 0.0 | | |
| | | Identities = 356/362 (98%) | | |
| | | Strand = Plus/Plus | | |
| | Description | DNA ladder | | |
| Clone # | T7 sequence | AAAGGAGCAACGCGGGTCTTCCCGCTGTTGCTTNTCGCGGCCACGGCCGAGCATACGTCCCCGGCCTGAGGTGGNG | | |
| 47 | | GTGGCGAGCCCACCGCCGTTTGCTGCGACCTCATGGAAGGTGGCGGAGGAAGTAGCAACAAATCCACCAGCGGGTT | | |
| | | AGCTGGCTTCTTCGGAGCCGGAGGAGCGGGTTACTCGAACGCTGATTTGGCCGGCGTCCCGCTGACTGGTATGAA | | |
| | | CCCCCTGTCTCCTTATTTAAATGTGGATCCACGCTATCTCGTTCAGGATACTGATGAATTTATTTTGCCAACTGGAG | | |
| | | CTAATAAAACCCGAGGCAGATTTGAACTAGCTTTCTTTACCATTGGAGGATGTTGCATGACAGGGGCCGCATTCGG | | |
| | | GGCAATGAACGGTCTTCGTTTAGGATTGAAGGAAACCCAGAGCATGGCCTGGTCCAAACCAAGAAATGTACAGATT | | |
| | | TTGAATATGGTGACTAGGCAAGGAGCACTTTGGGCTANTACTCTAGGCTCCCTGGCTTTGCTCTATAGNGCTT | | |
| | BGH sequence | CTGTTTTCACCCTTTATTTGGAAAACAGGGCAACATTTAAGTTTTAGATTTTTTAAATGAATTAACATGGTAATAAAA | | |
| | | AGTATGGCCTGAATTNGGAGAGTAAGNGGTTTTCCAGT | | |
| | Identity | dbj\|AB006451\|AB006451 Rattus norvegicus mRNA for Tim23. complete cds | | |
| | | Length = 1102 | | |
| | | Score = 791 bits (399), Expect = 0.0 | | |
| | | Identities = 492/523 (94%), Gaps = 1/523 (0%) | | |
| | | Strand = Plus/Plus | | |
| | | gb\|AF030162\|AF030162 Homo sapiens inner mitochondrial membrane translocase Tim23 (TIM23) mRNA, nuclear gene encoding mitochondrial protein, complete cds | | |
| | | Length = 841 | | |
| | | Score = 484 bits (244), Expect = e−135 | | |
| | | Identities = 384/431 89% | | |
| | | Strand = Plus/Plus | | |
| Clone # | T7 sequence | AAAGGGCCTCCGCACTNCCAAGTCATTTGNCGGCTACNNGNCTATNAGNGCANAGCAGGGTGTCANGGACCNAGTT | | |
| 48 | | GCTNNAGGCNGNNAAGAGCCNGCTGCAAGGTNAATCCTACAGTACGTGNAAACTCTGATGGAAGNGATNCCCAAGAT | | |
| | | CTGNCGNCTTCCGCNACATGAGTACGGCTCACCTGGCATCCTGGAGATCTTCCACCNCCAGCTGAAGGACATTGAGG | | |
| | | AGAATGCACNAGCTGAAAACCGTATGCTTCCAGAACCTGCGGCAGGTGGGAAATGCTGCCCTCTTCNGCCNGCTTAT | | |
| | | ANGAGCANAGNCTGNCTTTAGAANAANTCTGTGACCNGTTGCATGCAGCTCNTTTCCAGAATNTCTTACCTNNGAAT | | |
| | | CCNTGNANAAGAGGGNNAGAGAGTNTGAATNCCNAAATNAACAGACTNCAATCCAANTATTGCCCCANTGTACCCTN | | |
| | | GCCCCAATGNTTGAAANGGCTGNGGACCCCNAGCAAANCGCAACTGCNCGANACGGNGNCTNGCTGNCCAANGNANC | | |
| | | GCCTCATGNNGCGGCATGNCCATGTTTGAAGTTCNCCTNACNCNCNCCNCANCTTCTGGCNTGATCCCCNATCNGGC | | |
| | | GCNGNCCCCCTCACCNAGCNNTTGGTGNNATNCNNGCNCNANAGANGCCTTGNGCTTCNCANCCTCTAGCTAACCCCC | | |
| | | NGCCCCTNNGTATNNCTTCACTNC | | |
| | BGH sequence | AATACAATTAGTTGGTATTATGCTCGTACAGGATGANCGACCCCANTNTCCCNTCNTANCTGCTGTAATATTCGGCA | | |
| | | TGAAAATACTTGTTAATACCGTAANGGCAACAACTAGTAACCGTATTCTCAGACTTCCCAATTCCAAAGGCATATACA | | |
| | | ATTTTAGTATAGAAAAATAAGTAAATTTTATAAAGTTAAGCTTTCAGATCAAAAGTAGGTtCAGACATAANGGAAAAT | | |
| | | AGCCCCTAAAAATTTCAATATAGTTTACATAAAGACAAACATGCCATCAGTTACTGGGATGCTGNGAGCTTAGCCCT | | |
| | | CAGCTACNGGCTAGGGACTGGAGGANGGGNGGCTGGAAGCAGCGTACATGCTCCACAGGNGNGCTCTCGCCATCAC | | |
| | | CAGATTTCAAGTACTTGTCCAGGATAGTGATGATTTCATCGTTGAGAATGTGGAACTTGCGGATCCTCTCCACCATC | | |
| | | TTCTTCAATGGCACATTTTTGATGATCTCATCTTTGCCATCATGNTTCTGAACTTTGAGANGATGATAGCAGAAATC | | |
| | | CAACACAGCAAAGCGCCGCTGNTGTCCAAGANGTACAATGATCATGCAGCCAGCCCAGAGGANGCCCATCTCCAAAA | | |
| | | NCACNGCTCCACTTGTAAAACTCGNGGGGCCCCCTACCGNNAAAT | | |
| | Identity | gbj\|AF072697\|AFG72697 Mus musculus SHYC (Shyc) mRNA, complete cds | | |
| | | Length = 4112 | | |
| | | Score = 983 bits (498), Expect = 0.0 | | |
| | | Identities = 551/571 (96%), Gaps = 2/571 (0%) | | |
| | | dbj\|038549\|HUMHA1025A Human mRNA for KIAA0068 gene, partial cds | | |
| | | Length = 4379 | | |
| | | Score = 289 bits (146). Expect = 3e−76 | | |
| | | Identities = 254/293 (86%) | | |
| Clone # | BGH sequence | AATCTGGAACACAAAAGTTTATTAGAGTAAAAATACATATATAATATCCAACATTAAAATTATCTCATGTCACATGTT | | |
| 49 | | TTACCTGGATTAGTTTTTCCAAAAAAATGCTTTAAAACTCTATGCCTTAAATAATTTTGCATATATGTAAACAAATCT | | |
| | | TAGATTACCGAAGATGCCATTATACCTGTTAGATATTGAACATNAACCTTTAGGAATGGGAACTACAAGTTTCACTC | | |
| | | TATTACACTAAGCGCTACTCTGAAGGAAGGAGGGAAGGACGGAAGGAAGGACAGAAGGAAGGATAACCCACTTGAG | | |
| | | ATGAGGTAAGAGTAAGAGTTTAGTACCAAATGTTGACACAAGAATGTTAAAGGCATTCATGGAAAGTCACTCCGCT | | |
| | Identity | gb\|U07971\|RNU07971 Rattus norvegicus Sprague-Dawley L-arginine:glycine amidinotransferase mRNA, partial cds. | | |
| | | Length = 2260 | | |
| | | Score = 198 bits (100), Expect = 5e−49 | | |
| | | Identities = 212/247 (85%), Gaps = 16/247 (6%) | | |
| | | Score = 85.7 bits (43), Expect = 6e−15 | | |
| | | Identities = 49/51 (96%) | | |
| | | Strand = Plus/Minus | | |
| Clone # | T7 sequence | AAAGGCTGCTGTCAGGNGGTCCCTTTTATGGATGGGCTCCTGNGGNCGCTGCGCAGNGGNTGNTCGACTTCCGNAG | | |
| 50 | | NGNCNCTCGGGCCANCGAGCGCCGTCTTTANCNAGCTGCCANGGCTGTGGCCGCTGTCGGCCGCCNGAGAGCCCTG | | |
| | | CGCTGTCCGCTGNAGCTCCTGCTGTNACTCCTGNTGNNAGCCGCCCTGCGCTGNGCNGTAACGACCCTGANAGAATA | | |
| | | CTCTTGCGGTATGTGAAAGCTCTTACCCTCTACTCCGACCGCTACACCACCTCCNNGGNGGCTGNACCCTATCCCAC | | |
| | | AGTTGAAGCGTGNAGGAGGCACCGANGNNTGANACGNCCTATACCCCAGGGNNNATACAGTGCNTNACTACAGGNT | | |
| | | GNNNANCCTACCTATGACAGTGCGAACTNTNAGACTCGCCTCT | | |
| | BGH sequence | CTNNATNGNCTTCCAGNACNGNANNTNGCNAGCCTCAAACCANNAANGNAANACNACGNTGNANAGNGANGNNCAGNA | | |
| | | GACNGAAGGANCTNATCANNNCGANNNATGGANGANNTAGNTANCACAANANGGNCNGNTNACGNNGGAGCTGNAGG | | |
| | | ANGACGGNANCGAGGCGGACAGATNAGNAGT | | |
| | Identity | dbj\|AU035342\|AU035342 AU035342 Sugano mouse brain mncb Mus musculus cDNA clone MNCb-0343, mRNA sequence [Mus musculus] | | |
| | | Length = 718 | | |
| | | Score = 345 bits (174), Expect = 3e−93 | | |
| | | Identities = 286/327 (87%), Gaps = 4/327 (1%) | | |
| | | Strand = Plus/Plus | | |
| | | gb\|AI286459\|AI286459 ui77d03.y1 Sugano mouse liver mija Mus musculus cDNA clone lMAGE:1888421 5′ similar to WP:T06D8.9 CE02330; mRNA sequence [Mus musculus] | | |
| | | Length = 456 | | |
| | | Score = 345 bits (174), Expect = 3e−93 | | |
| | | Identities = 286/327 (87%), Gaps = 4/327 (1%) | | |
| | | Strand Plus/Plus | | |
| Clone # | T7 sequence | AAAGCCTGTGTACGCCACCATCGGCTTTNGGTATCGNCAACACGGCCTTCANTGNGGAGTCGCTGNTTGTNGTAGA | | |
| 51 | | GCGAGCTGGACGACGGACCCTGCACCTCATTGGCCTGGCTGGCATGGCAGGCTGAGCTGNGCTCATGACCATCGCC | | |
| | | CTGGCCTTGCTGGAACGGCTGCCTTGGATGTCCTATGTGAGCATCGNGGCCATCTTTGGCTAAGNGGCCTTCTTTG | | |
| | | AAGTAGGCCCTGGTCCTATTCCATGGCTCATTGTGGCCGAGCTGNTCAGCCAGGGGCCCCGTCCTGCTGCTATNGC | | |
| | | NGAGGCTGNCTTCTCCAACTGGACCTCAAACTTCATTGTGGGCATGNGCTTCCAGTNTGNGGAGCAACTGNGCGGN | | |
| | | CCCTACGTGCTTCATCATCTTCACGGAGCTCCTCGNGCTCTTCTTCATCTTCANCTACTTCAAAGTCCCGAGCCAA | | |
| | | AGGCCTGAACCTTCNATGAGANCGCTTCCGNCTTCCGGCAGGC | | |
| | Identity | gb\|M22998\|MUSGLUTRN Mouse facilitated glucose transport protein mRNA, complete cds. | | |
| | | Length = 2473 | | |
| | | Score = 739 bits (373), Expect = 0.0 | | |
| | | Identities = 464/498 (93%), Gaps = 3/498 (0%) | | |
| | | Strand = Plus/Plus | | |
| | | gb\|S77924\|S77924 Glut-1 = glucose transporter isoform 1 [mice, embryo, mRNA Partial, 321 nt] | | |
| | | Length = 321 | | |
| | | Score = 460 bits (232), Expect = e−127 | | |
| | | Identities = 289/310 (93%), Gaps = 2/310 (0%) | | |
| | | Strand = Plus/Plus | | |
| Clone # | T7 sequence | AAAGCTTACCCAGCAGGGNGAGAGATTTNTATCAACAAGAGAAGGCACGGANGGGCTCAGCGGGACTCAGANTNT | | |
| 52 | | NGCCTNGGNGNCNGGTCCGGAACNGGGGCAGCTGACCTTCCTTGGCCNGGACGGAATCATNGACCCTCCTCGAACN | | |
| | | GGNGNGAAGGAAGCTGNCACAANANTCATNGCCTCAGGAGTGTCCATTAAAATGATCACTGGAGATTCTCAGGAGAC | | |
| | | NGCAANTGCCATCGCTAGACGNCNGGGATTGTACTCTAAGACTTNACAGTCCGAGNCTGGGGAAGAAGTNGATACA | | |
| | | ANGGAGGNGCAGCACCTTTCACAGATAGAGCCAAAGGNNGTAGTATTTTACAGAGCAAGCNCAAGACACAAGATGAA | | |
| | | AATTATTAAGTCTCTACAGAAGAACGGG | | |
| | BGH sequence | GCCTTTAAANGATTGAGTTNATTTGTGNGTTAGTAAGAAAGCCCTATAACCATAAATAGTNCAATATTTAAAAGTAAA | | |
| | | AAAAATATTTATATCCATCTAAGACAGACAGTGTATTNGTNCATTAGAATTCTTTAAGTGCAGAAGGTGGNTCAGGN | | |
| | | TTNGCCTTTNGNATTTTTTAATTCAGTACTTGCCCATATGTTGAAGTTCACTNAATTGNAAAAGCCAATTNAAATCAA | | |
| | | GGAATGTGTTATTNAAAGNTGCNTAATTTCATTNGGGACTACCAACAAAGTTNAAATCAAGNCTTTAACATTAANGG | | |
| | | TCATTTTTAGTNAANGGAAAGTTAAAAAGTCCTTAAGTCTTCATTATCTTCTTGAACTTGCCCTTCTAAATGATTCTN | | |
| | | AGACTGCANTNCCTAGTTTGCNAATTAAAAAAAAATGCNNTATGCATCATACTTNAAGGAAAGACGATGAANGCTNAA | | |
| | | CCAGCNTTCTTCGTGGACCTTCTCCCGGCTCCTTTCANCCTTCTTTATAACCCTCAGNCACANTGCACACTGACNAGG | | |
| | | TAAGGCCCNAAAGAANCANCAGATCCCNGNATNCTCAGGCTCTCCGNTTTTNAAAAACCTT | | |
| | Identity | emb\|AJ010953\|HSA010953 Homo sapiens mRNA for putative Ga2 + -transporting ATPase, partial | | |
| | | Length = 2175 | | |
| | | Score = 208 bits (105), Expect = 6e−52 | | |
| | | Identities = 311/390 (79%) | | |
| | | Strand = Plus/Plus | | |
| | | Convention for evaluating the phenotypical ‘cell death effect’: | | |
| | | − negative, −/ + and −/++ weakly positive, + positive, ++ strongly positive, +++ very strongly positive | | |
| Clone # | T7 sequence | GCTTGACCTTGGAGGCCTCAGCCTGAGACCTCAAAGCAGCCTCCAGAACTCCGGCAGAGTTCCTCTGTCTCGTCTTGC | | |
| 53 | | CGATTGAAGGTCCCCGTTTCTCCAATTTCTCTCCATCTTCTGGGAGGTAGCAGGAAATCAGAATCATGGTTGGTTTCA | | |
| | | AGGCCACAGATGTGCCCCCAACAGCCACTGTGAAGTTCCTGGGGGCTGGGACAGCTGCCTGCATTGCAGATCTCATC | | |
| | | ACTTTCCCTCTGGATACCGCCAAGGTCCGGCTGCAGATCCAAGGGGAGAGTCAAGGGCTAGTGCGCACCGCAGCCAG | | |
| | | CGCCCAGTACCGTGGCGTTCTGGGTACCATCCTAACCATGGTGCGCACTGAGGGTCCACGCAGCCTCTACAATGGGC | | |
| | | TGGACGCCGGCCTGCAGCGCCAGATGAGCTTTGCCTCCGTCCGCATTGGCCTCTACGACTCTGTCAAACAGTTCTACA | | |
| | | CCAAGGGCTCAGAGCATGCAGGCATCGNGAGCCGCTTCTGGCAGGTAGCACCACAGGTGCCTTGNCGTGGCTGTNGC | | |
| | | CCNAGCCTACAGATGTNNAAAGGCCCGTTTC | | |
| | BGH sequence | n.n. | | |
| | Serial clone | #6 | | |
| | number: | | | |
| | ACCESS No.: | U94593 | | |
| | | LOCUS MMU94593 1396 bp mRNA ROD Apr. 27, 1997 | | |
| | Definition: | Mus musculus uncoupling protein homolog (UCPH) mRNA | | |
| | Effect: | +++ after 24h | | |
| Clone # | T7 sequence | n.d. | | |
| 54 | BGH sequence: | GCCAGCCTAACACTTCTATGACAAACCGANGAAAATTATAAAAACTTTTCAAATATATTGCTCCTCAGACCCCCTATCC | | |
| | | ACACCATTAATTATTTTAACAGCCTGATTACTGCCACTAATATTAATAGCTAGCCAAAACCACCTAAAAAAAGATAATA | | |
| | | ACNGTACTACAAAAACTCTACATCTCAATACTAATCAGCTTACAAATTCTCCTAATCATAACCTTTTCAGCAACTGAACT | | |
| | | AATTATATTTTATATTTTATTTGAAGCAACCTTAATCCCAACACTTATTATTATTACCCGATGAGGGAACCAAACTGAA | | |
| | | CGCCTAAACGCAGGGATTTATTTCCTATTTTATACCCTAATCGGTTCTATTCCACTGCTAATTGCCCTCATCTTAATCC | | |
| | | AAAACCATGTAGGAACCCTAAACCTCATAATTTTATCATTCACAACACACACCTTAGACGCTTCATGATGTAACAACTT | | |
| | | ACTATGGTTGGCATGCATAATAGCATTTCTTATTANAATACCATTATATGGAGTTCACCTATGACTACCAAAAGCCCAT | | |
| | | GTTGAAGCTCCAATTGCTGGGTCAATAATTCTAGCAGCTATTCTTCTAAAATTAGGTAGTTACGGAATAATTCGCATC | | |
| | | TCC | | |
| | Serial clone | #7 | | |
| | number: | | | |
| | ACCESS No.: | M27315.1 GI:343181 | | |
| | Definition: | Rattus norvegicus mitochondrial cytochrome c oxidase subunits I,II | | |
| | | and III, and ATPase subunit 6 genes, complete cds, and Trp-, Ala-, | | |
| | | Asn-, Cys-, Tyr-, Ser-. Asp-, Lys-, Gly-, Arg-tRNA genes. | | |
| | LOCUS: | RATMTCYTOC 7632 bp DNA ROD May 29, 1996 | | |
| | Effect: | ++ | | |
| Clone # | T7 sequence | GGATTGGCGGGGTTCTCTTTGACCACATAAATCAATTCTTTCTTTTGGGAGCCTCAATGGTGGCAACTGCAGCGGGA | | |
| 55 | | CTTTATCTCATCCCGTTCTGCAAGACAGCAGTCTTATTGATCATCACCATGTCTGrCTTTGGAGCTTCCGTTGGTGTT | | |
| | | GTGGATACAGGTGCAAATGTTCTCATCTTGGATCTCTGGGGGGACAAAGGAGCCCCACAAATGCAGGCCTTGCACTT | | |
| | | CAGTTTCGCCTTGGGTGCCTTTCTGGCTCCCCTGCTGGCTAAGTTGGCCTGGGGTACAGCACCTGCTCAGAACCACA | | |
| | | ACATGAATCTGCTGNGGGCATATGCTTCCATTGGCACCTTTATTTTAGTAGTTTCTGTCTTTCTGNNNGGTCTGTTTT | | |
| | | GTAAGAAACATTCAAGGCAGAAAAAACCCAGAGCATCTGCTGAGGGAGCTCGAAGGGCTAAATATCACAGGGCCCTG | | |
| | | CTATGCCTGCTCTTCCTCTTCTTCTTCTTCTATGTGGGAGCCGAGATAACATACGGCTCTTACATATTCTCCTTCGCCA | | |
| | | CCACCCATGTTGGCATGGAAGAGAGCGAGGCAGCTGGCTTGAATTCCATCTTCTGGGGAACCTTTGCAGCCTGCAGG | | |
| | | GGCCTGGCCATCTTCTTTGCGACATTCTTACAGCCTGGAACCATGATCGTGCTGAGCAACATTGGCAGCCTGGNCTCA | | |
| | | TGTTTCTTTCTGGTACT | | |
| | BGH sequence: | AAATATCACA GGGCCNTGNT AGCCTGCTNT TCCCTCTTCT TCTTCTTCTA | | |
| | | NTGGAANCCG AAGNTACCAT NNCGNTTCTT ANCAGTTTCT CCTTTCGGCC | | |
| | | NCCNACCCAG TGGCATGGAA GAGAGCGAGG CAGCTGGCTT GAATTCCCAT | | |
| | | CTTCTGGGGA ACCTTTGCAG CCTGCAGGGG CCTGGCCATC TTCTTTGCGA | | |
| | | CATTTCTTAC AGCCTGGANC CATGATCGTG CTGAGCAACA TTGGCAGCCT | | |
| | | GGTCTCATGT TTCTTTCTGG TACTTTTTGA CAAGAGCCCT GTTTGTCTCT | | |
| | | GGATCGCGAC TTCTGTGTAT GGAGCCTCAA TGGCAGCCAC GTTTCCCAGC | | |
| | | GGCATCTCCT GGATTGAGCA GTACNCCNCC TTAACTGGGA AATCTGCAGC | | |
| | | ATTCTTTGTA ATTGGCTCTG CCCTGGGAGA TATGGCCATT CCAGCGGTGA | | |
| | | TCGGAATTCT TCAGGGACNC TACCCAGATC TGCCAGTAGT TCTGTACACA | | |
| | | TGTCTGGGCT CAGCCATATT CACAGCTATT TTATTTCCTG TGATGTATAA | | |
| | | ATTAGCTACC TTGCCCCTGA AGAGAGAGGA CCAGAAAGCT TTGCCCACTA | | |
| | | GTTCTAGACT GTGAGGAAGA GACTACATGA GAACTTAAAA AAAAAAAAAA | | |
| | | AGGGCGGCCG CTCGAGCATG CATCTAGAGG GCCCTATTCT ATAGTGTCAC | | |
| | | CTAAATGCTA GAGCNCNNCT GTCA | | |
| | Total sequence | GGATTGGCGGGGTTCTCTTTGACCACATAAATCAATTCTTTCTTTTGGGAGCCTCAATGGTGGCAACTGC | | |
| | combined (from T7 | AGCGGGACTTTATCTCATCCCGTTCTGCAAGACAGCAGTCTTATTGATCATCACCATGTCTGTCTTTGGA | | |
| | and BGH) | GCTTCCGTTGGTGTTGTGGATACAGGTGCAAATGTTCTCATCTTGGATCTCTGGGGGGACAAAGGAGCCC | | |
| | | CACAAATGCAGGCCTTGCACTTCAGTTTCGCCTTGGGTGCCTTTCTGGCTCCCCTGCTGGCTAAGTTGGC | | |
| | | CTGGGGTACAGCACCTGCTCAGAACCACACCGAGTCCGACCTTGACACTCTGATGCTGAACCGATCCTCC | | |
| | | AACGGCACCTCAGACTCCGTGTTTGCGGTACCCGATGACATGAATCTGCTGNGGGCATATGCTTCCATTG | | |
| | | GCACCTTTATTTTAGTAGTTTCTGTCTTTCTGNNNGGTGTGTTTTGTAAGAAACATTCAAGGCAGAAAAA | | |
| | | ACCCAGAGGATCTGCTGAGGGAGCTCGAAGGGCTAAATATCACAGGGCCCTGCTATGCCTGCTCTTCCTC | | |
| | | TTCTTCTTCTTCTATGTGGGAGCCGAGATAACATACGGCTCTTACATATTCTCCTTCGCCACCACCCATG | | |
| | | TTGGCATGGAAGAGAGCGAGGCAGCTGGCTTGAATTCCATCTTCTGGGGAACCTTTGCAGCCTGCAGGGG | | |
| | | CCTGGCCATCTTCTTTGCGACATTCTTACAGCCTGGAACCATGATCGTGCTGAGCAACATTGGCAGCCTG | | |
| | | GTCTCATGTTTCTTTCTGGTACTTTTTGACAAGAGCCCTCTTTGTCTCTGGATCGCGACTTCTGTGTATG | | |
| | | GAGCCTCAATGGCAGCGACGTTTCCCAGCGGCATCTCCTGGATTGAGCAGTACNCCNCCTTAACTGGGAA | | |
| | | ATCTGCAGCATTCTTTGTAATTGGCTCTGCCCTGGGAGATATGGCCATTCCAGCGGTGATCGGAATTCTT | | |
| | | CAGGGACNCTACCCAGATCTGCCAGTAGTTCTGTACACATGTCTGGGCTCAGCCATATTCACAGCTATTT | | |
| | | TATTTCCTGTGATGTATAAATTAGCTACCTTGCCCCTGAAGAGAGAGGACCAGAAAGCTTTGCCCACTAG | | |
| | | TTCTAGACTGTGAGGAAGAGACTACATGAGAACTTAAAAAAAAAAAAAAAGGGCGGGCGCTCGAGCATGC | | |
| | | ATCTAGAGGGCCCTATTCTATAGTGTCACCTAAATGCTAGAGCNCNNCTGTCA | | |
| | Serial clone | #13 | | |
| | number: | | | |
| | ACCESS No.: | AL080317.11 GI:5830430 | | |
| | Definition: | Human DNA sequence from clone RP5-1112D6 on chromosome 6q21-22.2. | | |
| | | Contains the gene for a PUTATIVE novel protein similar to bacterial | | |
| | | NARK (nitrite extrusion protein, nitrite facilitator), the 3′ end | | |
| | | of the REV3L gene for REV3 (yeast homolog)-like, catalytic subunit | | |
| | | of DNA polymerase zeta (EC 2.7.7.7, POLZ), ESTs, STSs, GSSs and a | | |
| | | putative CpG island, complete sequence. | | |
| | LOCUS | HSJ1112D6 135305 bp DNA PRI Feb. 21, 2000 | | |
| | Effect: | | | |
| Clone # | T7 sequence | TGCAATGGCGGACGTGTCTGAGAGGACGCTGCAGGTGTCCGTGCTAGTGGCTTTCGCCTCTGGAGTGGTCCTGGGC | | |
| 56 | | TGGCAAGCGgATCGgcCGTGGAGGCGTTACCTAGACTGGAGGAAGCGGAGGCTGCAGGTCGGGCTGGCAACGACTC | | |
| | | AGAAAAAGCTGGACCTGGCCTGAGCACGCGCTGCAGCCCGAGTCCGCCGGGTTCTCACTCCCTAAGCCCAACGCAGC | | |
| | | CCGGATCGTGGGAGCCGCGCGACCCAGGAGTCGTCCTTGCACGGCTTGCAAGAACATGGCTTGCTTCAGAAAGAAAA | | |
| | | TAGTTTTGTCTTCTGTAACAACTTACTTTCAGCTTGTCGAAGATGAAAATAAAAAGCACTGGAGAGAAATAATTTCTT | | |
| | | GCACTTTATGAATCTATTTTTAAAATAAAAAAATTAAACATCTTTGAATCTTTTTCCTCCTCACAAAAGAAAGCAGTAT | | |
| | | TTTTGCCTACCATTCAgTTTGCTGCAGTAAGAGATTTGGAGCCTGAAAGCAGAGACTTTCTGATG | | |
| | BGH sequence: | TGAGGCTGCA GGCCAAGCTG GCAACGNCTC AGAAAAAGCT GGACCTGGCC | | |
| | | TGAGCACGCG GCTGCAGCCC GAGTCCGCCG GGTTCTCACT CCCTAAGCCC | | |
| | | AACGCAGCCC GGATCGTGGG AGCCGCGCGA CCCAGGAGTC GTCCTTGCAC | | |
| | | GGCTTGCAAG AACATGGCTT GCTTCAGAAA GAAAATAGTT TTGTCTTCTC | | |
| | | TAACAACTTA CTTTCAGCTT GTCGAAGATG AAAATAAAAA GCNCTGGAGA | | |
| | | GAAATAATTT CTTGCNCTTT ATGAATCTAT TTTTAAAATA AAAAAATTAA | | |
| | | ACATCTTTGA ATCTTTTTCC TCCTCACAAA AGAAAGCAGT ATTTTTGCCT | | |
| | | ACCATTCAGT TTGCTGCAGT AAGAGATTTG GAGCCTGAAA GCAGAGACTT | | |
| | | TNTGATGGAA TCTCNCCTTG GTACAGCCTG GAGGCAGATN TGATCAACGG | | |
| | | ACCATTATGA GTCATTTTTC TAGACATATT CAGAAAACCT AGGAGCTGTG | | |
| | | TCAAATGCCT GAATTAAGCA TTACAAATGC AAGATATTTG CNCTTTGAAG | | |
| | | AATGTAGAGA GTAAAAAAAC TAAAATTAAA AAAAATAATG CATGTGATAT | | |
| | | AACGGAATAT ATATGTGAAA GAGAAAAAAA AAAAAAAAAA AAAAAA | | |
| | Serial clone | #20 (EST) | | |
| | number: | | | |
| | ACCESS No.: | AW106096.1 GI:6076832 | | |
| | Definition: | um23a10.y1 Sugano mouse embryo mewa Mus musculus cDNA clone | | |
| | | IMAGE:2225370 5′, mRNA sequence | | |
| | LOCUS: | AW106096 539 bp mRNA | EST Oct. 20, 1999 | |
| | Effect: | ++ | | |
| Clone # | T7 sequence | GATAGTTTTGAACTTCCGATTCCCCTGCCTCCCCACCCCAGGGCTGTGATTGCAGGTGTGCGCTTGGGACTGACCCC | | |
| 57 | | AGGCTTTGTCGGTGCTAGGGCAGCACTCTGCGACTGAATTAGGTCCCAGCCACTTCTCTGTCTTTTAAAAGAACAAAA | | |
| | | CATTGCTAAATGTGCCATTGTTGCTTTGAGTTTTAATTCTTTTTTTTTTCTTTCTTTCATAAAACATTACAGTCTTAAG | | |
| | | ATATTAAAGACTTTTATTCTGGTTCTATTTCTGTCTTTTCACTCAAAACTGGTTTTACAAATGATGCCTTGTTTACAGA | | |
| | | AAGCTCTCTACCACAGGGCCTAGTCATGTGTAAAGTCTCAGTTTCTCTCTGGAGTATCTTGGAGCCTAGCACACTGGC | | |
| | | TTTAAAGGACACAGCTAAGAAGCTGATATCTTGACAGTGTTTGTAGACCTTTGTTATAAAAATGAATGTCCTGGAAAG | | |
| | | GGTTGGGAGGGAGTTCAACAACAAAGAAACAAGAATGTCATGTTTAAATTTAATAGTTGTCTAAAATGTCATCTCAAG | | |
| | | TCAAGTCACTGGTCTGTTTGCATTTGATAGGTTTTTATACTAACTAGCATTATAAGATTATTTCATAATTAGAAAATAC | | |
| | | CTGTGGATATTTGTATAAAAGTGTGAAATAAATTTTTTACAAAAGTGCTCATCGCTTGTTAACACAGCATCATGTATG | | |
| | | TGAAAGCAAACTCTAAGATTATAAATGACAACCTGAGTTGCCTTTCTTTGTATTTCATCAAGCCNAAGTAAAGCTTTCA | | |
| | | NTATTTAAA | | |
| | BGH sequence: | n.d. | | |
| | Serial clone | #21 | | |
| | number: | | | |
| | | EST with low homology to human integrin | | |
| | ACCESS No.: | NM_000210.1 GI:4557674 | | |
| | Definition: | Homo sapiens integrin, alpha 6 (ITGA6) mRNA. | | |
| | LOCUS: | NM_000210 5611 bp mRNA PRI May 15, 2000 | | |
| | Effect: | + | | |
| Clone # | T7 sequence | GCAANGGCGGACGTGTCTGAGAGGACGCTGCAGGAGTCCGTGCTAGNGGCTTTCGCCTCTGGAGTGGTCCTGGGGT | | |
| 58 | | GGCAAGCGAATCGGCTGCGGAGGCGTTACCTAGACTGGAGGAAGCGGAGGCTGCAGGACAAGCTGGTAACNACTCA | | |
| | | GAAAAAGCTGGACCTGGCCNGAGCACGCGCTGCAGCCCGAGTCCGNCGGNNTCTCACTCCCTAAGCCCAACGCAGCC | | |
| | | CGGATCGTGGGAGCCGCGCGACCCANGAGTCGNCCTTGCACGGCTTGCAAGAACATGGCTNGCTTCAGAAAGAAANT | | |
| | | AGTTTTGTCTTCTCTAACANCTAACTTTCNNCTTGTCCAANATGAANATACGNAGCNCTGGANAGANNTAATTTCTTGC | | |
| | | NCTTTANNAATCTATNNTTAAANTACANANATTNANCATCTTNGANTCTTTNTNCTNCTCACAAAAGANAGCAGCATTTT | | |
| | | TGCCTACCATTCAGTTNGCTGCANTAAGAGATNTGGAGCCNGAAAGCAGAGACTTTCTGATGGAATCTCACCTTGGAC | | |
| | | AGCCTGGAGGCAGATCTGATCAACGGACCATTNTGAGTCATTATTCTAGACNTATTCAGAAAACCTAGGAGCTGNGTN | | |
| | | AAATGCCTGANTTAAGCCTTACNAATGCNAGATNTTTGCACTNTGAANAATGTANNGNGTAAANAAACTACTATTAGTC | | |
| | | CAAATAATGCATGNGATNTAACGGAATATATATGTGAAAGAGAAAAAAANNNANNNAAAAAAAAAGGGCGGCCCGCTC | | |
| | | GAGCATGCATCTAGAGGGGCTATTCTATAGTGGCNCCTCAATGCTANAGCTTCGCTGAC | | |
| | BGH sequence: | n.d. | | |
| | Serial clone | #23 | | |
| | number: | | | |
| | | Mouse EST clone | | |
| | ACCESS No.: | AW106096.1 GI:6076832 | | |
| | Definition: | um23a10.y1 Sugano mouse embryo mewa Mus musculus cDNA clone | | |
| | | IMAGE:2225370 5′. mRNA sequence. | | |
| | LOCUS: | AW106096 539 bp mRNA EST Oct. 20, 1999 | | |
| | Effect: | + | | |
| Clone # | T7 sequence | AGGGCGGCCGCTCGAGCATGCATCTAGAGGGCCCTATTCTATAGNGGCACCTAAATGCTTAGAGCTCGCTTGATCAA | | |
| 59 | | GCCTCGACTGNGCCTTTTAAGTTTGCCAGCCATCTGGTGNTTNCCCCTCCCCCGGGCCTTTCCTTGACCGTTGGAAGG | | |
| | | NGCCACTTCCCAATTGNNCCTTTCCTAAAAAAAATGNGGGAAAATGGCAATCGCAATTGNCTTGGGTAAGGNGGCAAT | | |
| | | TCTAATTCTTGGGGGGGAT | | |
| | BGH sequence: | n.d. | | |
| | Serial clone | #25 | | |
| | number: | | | |
| | ACCESS No.: | NM_007748.1 GI:6680987 | | |
| | Definition: | Mus musculus cytochrome c oxidase, subunit VI a, potypeptide 1 (Cox6a1), mRNA. | | |
| | LOCUS: | NM_007748 531 bp mRNA ROD Jan. 4, 2000 | | |
| | Effect: | ++ | | |
| Clone # | T7 sequence | GCATCCCTCCATGGCCTCTGCATTGGCTTCTGCTTTCTGACCTGCTTGAGTTCCAGTCCTGACTTCCTTGGNGATGAA | | |
| 60 | | CAGCAGTATGGAAGTGTAAGCCGAATAAACCCTTTCCTCTCCAACTTGTTTCTTGGTCATGATGNNTGTGCAGGAATA | | |
| | | GAAACCCTGACTAAGACAAATTGGTACCAGCAGAGTGGGGTATTCCTGTGACAACCTGACCATGTTTTGGGGAGGAC | | |
| | | TGTGGATGGACTTTGGAACTTTGGGCTTAAAGATCCATCCGNTGNTAAGAGCTCTGTCAGATGTTGTGTAGGAGCTT | | |
| | | GGAAGATAATGTTGAGAACACTGNAGAANATGGAGGNCTGGTNTGNGAAATTTNAGAGGGAAAANTAAAGACTCTTT | | |
| | | CAGGGCCATTGCTGTTTTGAATGTGAAGATTCTGTAGNNCTGGATAGCTGGGGCTNAANAATCANCTGTGNTTAACA | | |
| | | NGATCCAGAACTACCAANGCANAAACTTTGCATTACTGGGACTATTGATGCNTNNNTAGCTGNAGCTAACAAATTANC | | |
| | | GGTGATTAANAAAGAGACCAGCNTCATTNAGGGTGACATCTTTCTGGGNANGTGTTTGNTGAAAGCACANNGATNCNT | | |
| | | GTGNTTNCAGAGATGGCCAAANGCTGTACCNCCTGCTTGCAANNANGGACTTGGGNATAANTGTAANNGGGTCNACCC | | |
| | | NCNTNGNTACTGGGGTTTTTTAANGGCCNTTTAACNGGGNTCNACGGCCACAGNCANCCTTCAGGGCCTNNGGCANCC | | |
| | | TGGTTAAAAANGCCCCATTNNGAAANGGNCCAAATTGNCTTTNAAAGGGTACNAGGCCTNCAAACTTGCCCAAATTGT | | |
| | | ANAGGGCCNCCCGGG | | |
| | BGH sequence: | GACCTGGCTT TNGCNCGCNN TNCANCCCTA GTCCCCCGGG TTCTCACTCC | | |
| | | CTAAGCCCAT CGCAGNCCGG NTNGTGGANC CGCGCGTCCC AGGNTTCGTC | | |
| | | CTTNCNCGGC CTNCAAGAAC ATGGCTTGCT TCAGAAAGAA AATAGTTTTG | | |
| | | TCTTCTCTAA NAACTTACNT TCAGCTTGTC GAAGATGAAA ATAAAAAGCC | | |
| | | CTGGAGAGGA ATAATTTCTT GCNCTTTATG AATCTATTTT TAAAATAAAA | | |
| | | AAATTTACCN NCTTTNAATC TTTTTCCTCC TCNCAAAAGN AACCAGTATT | | |
| | | TTTGCCTNCC ATTCANTTTG CNNCANTAAG ANNTTTGGAG CCTGAAACCN | | |
| | | NAGNCTTTNT NANGGANTNT CNCCTTGGTT CAGCCTGNAG GCAAATCTGA | | |
| | | TCAACGGACC TTTATGAGTC ATTTTTCCTA GACATATTCA GAAAACCTAG | | |
| | | GAGCTGTGTC AAATGCCTGA ATTAAGCATT ACAAATGCAA GATNTTTGCN | | |
| | | CTCTTGAAGA ATGTAGAGAG TAAAAGAACT ANAATTAAAA ANAATAANGC | | |
| | | NTGTGATATA ACGGAATATA TATNTNAAAA ANAA | | |
| | Serial clone | #26 | | |
| | number: | | | |
| | ACCESS No.: | AC005403.1 GI:3402732 | | |
| | Definition: | Mus musculus clone UWGC:ma53a068 from 14D1-D2 (T-Cell Receptor Alpha Locus), complete sequence. | | |
| | LOCUS: | AC005403 40638 bp DNA ROD Aug. 7, 1998 | | |
| | Effect: | ++ | | |
| Clone # | T7 sequence | GCCGCTGNAGGANGGGACCCAATNCTCTATTANCATCTGNNCTGATTCTTNGGGCACCCNGAANTTTATATNCNTATC | | |
| 61 | | CTCCNAGGATNTGGAATTATTACACATGTCGTACTTACTACTNCGGANNAANANAACCTTNCNGCTATANANNANTNTG | | |
| | | TATGAGCANTANTGTCNATNGNCTNTCTAGGCCTTTATNGNNTGAGCCCACCACATATTTACAGTANGNTTAGNTGTA | | |
| | | NACACACGANCNTACTTTACATCANCCCGNTNTNATNATCNGCANTTNCTACCGGNNTCAAANTNTTTANCTGACTNGT | | |
| | | AACCCTACACGGAGGTNNNNTTAACTCGATCTNCCAGCTATACNANTGAGCCTTTANGCTTTATNTTCTTATTTACNGT | | |
| | | NGGNGGTCTAANNGNAANTTGCNTNANCCNANNTNATCCCCTTGANCATNNCNCATTCACNGAACCANNCTANNCAGTA | | |
| | | TCCNCCATTTNCCNCTNTTGNNCCTATCANNTGGGGAGTCAGNTGCNCTGCTATTCNTATCCAGNNACNNTGTNTTCAT | | |
| | | CTNGAANNTCCCNNTTNNTNTNTTCANNGCCTTNCCACCCCTTNTTATAGGACCNCCNTCNNACNCNCNCAAGNCNTCNA | | |
| | | CTTTTCNCTTNNCTCANTATCTNCCNTNANGGCNTGTCTCANCNCCNTTNNTCCNTTTCTCTTTCCC | | |
| | BGH sequence: | n.d. | | |
| | Serial clone | #39 | | |
| | number: | | | |
| | ACCESS No.: | L07095.1 GI:3150274 | | |
| | Definition: | Mus domesticus strain NZB/B1NJ mitochondrion genome, complete sequence. | | |
| | LOCUS: | LOCUS MUSMTHYPA 16303 bp DNA circular ROD May 22, 1998 | | |
| | Effect: | ++ | | |
| Clone # | T7 sequence | GCAAACATGGNCAGGAGCATCTTGGCAGCNTTAAGCCTTCANAGAATTATCAACCANGGNCATNAGAGNCGACTCTGN | | |
| 62 | | CTCNTTCAGGCTTGCACCACCACCACAGGCNTAGGGGCCAGCACAGGCAGGGTCACTTANAGAGCTGAGACACCACA | | |
| | | GCAAGNGAAGGCTNGCACCTTTCACTTGCCCAGAGGANGCTCTGACNAAGGGGGTGCATCAACCANCTCCNGTGTAA | | |
| | | GCNGNCTAAGGAGTCCGAGGCAGCCCCCANCAGCTGCTGCTGTCACTGCCGCCACCTCATATTTGANAAGTCANGGTC | | |
| | | TGCANTATGCTTGACAGTNTGCGNAAAACCTCCCATCCTTATGTANCTGACAGGNGCTTTTNCGCGNANTNACAAAAG | | |
| | | CCACCTTGAACCCTGTCANTNCTAGGTCACCTTCNAGCTTGCCTNGACNNAAANTCNNGTCCNTTGAAACCCCCNNTGG | | |
| | | CANGCCCAAACCCCANNNTGAAGCTTNAGATNCTCAGGACNNCNNAANTTANANNNTGNCCATTTCNNACCTTANNNTT | | |
| | | AATTTNAANNCTAAGGNCATTACTCTTNTTCCCCCCAAACNTNTAACNCCTTANANAGNACCNCCNNCTCTTTCCAATTN | | |
| | | NTTNTNNCCAAATNGTNTNTNNACCAGGTCCCCCAANTCTCAANNTNTAAATTTCTNNCTGNCAAACNCTTANANANTAN | | |
| | | GNTTTCCCTNNCCNAACAANTTNTGNNCANACTNTNTNCGGNGNCCC | | |
| | BGH sequence: | n.d. | | |
| | Serial clone | #48 | | |
| | number: | | | |
| | ACCESS No.: | Selenoprotein P | | |
| | Definition: | NM_009155.1 GI:7110716 | | |
| | LOCUS: | Mus musculus selenoprotein P, plasma, 1 (Sepp1), mRNA complete cds. Sepp1 | 2075 bp mRNA ROD Jan 4, 2000 | |
| | Effect: | + | | |
| Clone # | T7 sequence | GTTTTGTTTGAAACGGCAACACTTCGGTCANGATATCTTCTACCAGACACCAAGGCGTATGGAGATAGAATANAAAGA | | |
| 63 | | ATGCTTCGCCTCAGNTTAAACATTGACCCTGAAGCACAGGNGGAGGAAGAACCNGAAGAAGAGCCTGAAGACACCTCA | | |
| | | GAAGACGCANAAGACTCAGAGCAGGATGAGGNAGAAGAGATGGATGCAGGNACAGAANAAGAAGAGNGAGGAAACAN | | |
| | | ATAAGGAATCTACNGANAAGGATGAATTGTAANTTATACTCTCGCTATGAATCCCGNGTGGAGAGGNAATGNGAAGTT | | |
| | | NTGAAGTCATTTCTTTTGAGAGACTTGNTTTGNATGCTTCCCCNNGCCTCCTTCTCCCCTGCNCTGTNAAATGNTTGG | | |
| | | GATTNTGGGTCACAGGAAGAAGTGNNTTTTTTANCTGNANTNTTTTTNNCATTCCTCCTGAATGTANATTNNGTNCTA | | |
| | | TTTAACTGACTATTGGCGTCNNAATCTTGTGNTGTGTNTNAACCCTCCCCANNCATCCCCANCTCCCCNACNTNCCCTC | | |
| | | CNCCCCTCCNCCCTCTCTCTCNNCTCCCCTCCNNGNNCNCNCCCCNCNCATCTTCNTNNACNNGNGNCTNCCCNCCNNNT | | |
| | | NTNCTNCCCNNTGCNCTCTCTANANNNGGGGNCCCCCTNAATTCCNTATTANCNTGNCCCCCCN | | |
| | BGH sequence: | n.d. | | |
| | Serial clone | #51 | | |
| | number: | | | |
| | | ER transmembrane protein | | |
| | ACCESS No.: | J03297.1 GI:193094 | | |
| | Definition: | Mouse ERp99 mRNA encoding an endoplasmic reticulum transmembrane protein. | | |
| | LOCUS: | MUSERPX 2759 bp mRNA ROD Jun. 12, 1993 | | |
| | Comment: | | | |
| | Effect: | + | | |
| Clone # | T7 sequence | GGAAAGGACGCGGNCCCCCGGGCTGCGCCGGGTCGCGGCGGNCCCCGCAGGCAGCCATGGCGGCGGGCGCCGGGG | | |
| 64 | | CCAGGCCGGcgCCGCGCTGGGTAAAGGCGCTAGGTGAGCCGCTCAGTGCCGCGCnnCTGCGGCGGCTGGAGGAGCA | | |
| | | CCGCTACACCGCGGTGGGAGAGTCGCTGTTCGAGCCGCCGCTGCAGCTTTACTGGACCTGGCTGCTCCAATGGATCC | | |
| | | CGCTCTGGATGGCCCCCAACACCATCACCCTCATCGGCCTCGCCATCAACCTGGTCACCACACTAGTGCTCATCTTCTA | | |
| | | CTGCCCTACAGTCACGGAGGAGGCACCATACTGGACATACCTTTTATGTGCCCTGGGACTCTTTATCTACCAGTCACT | | |
| | | GGATGCCATTGATGGGAAACAAGCCAGAAGGACAAACTCTTGCTCTCCCTTAGGGGAACTATTTGATCATGGTTGTG | | |
| | | ACTCTCTTTCCACAGTATTTATGGCCATCGGCGCTTCCATTGCTGTTCGCCTAGGAACACATCCTGACTGGTTGTTTT | | |
| | | TCTGCTCTTTCGTTGGGA | | |
| | BGH sequence: | CTTTTTGTTTNGGTGCCaCTATAgAATAGGGCCCTCTAGATGCATGCTCGAGCGGCCGCCCTTTTTTTTTTTTTTTTT | | |
| | | TTTTTTTTTTTTTTTTTTTTTTTTTTTTTTTTNGGTTAATTGGCTGAAAATTTTATAACATTTAACTAAAAATTAAACAT | | |
| | | GNTTCATATCTCCTTTCATGAAACAGCAGCAGCAAGAGATGGCAAATGTTCGAAAGNCTNTTCAATCCATGTTATTNT | | |
| | | GATGAGTNTTTGAANAAAGAACTTGAACCTGTTCCGGNGCTTGTTGACATGAAGTNTTGAANATGTTTAAATGAAGGN | | |
| | | GTNTTGAAATTTGCAGGCCCAAAGAAGTAAAGTNTNTCATCATATCAAATGAANAAATGACCATTGNTNTCCNCAGAAC | | |
| | | AACATATTCATNTATAAAATTATTAAAGNATTGGTNTAAAAANAAAANACCTGGCCCAANAAANACAGNGTNTTGAANA | | |
| | | NATAGTTCACTTTTCGTCATGGGAGCTATCACCAATTTTTGNGCAACTTTAGCAAAGACNCNTCCAAACNTTAAAGTNT | | |
| | | AAAGGCANGGNTGTTTT | | |
| | Serial clone | #9/3 | | |
| | number: | | | |
| | ACCESS No.: | AF047431.1 GI:5514630 | | |
| | Definition: | HomoHomo sapiens AAPT1-like protein mRNA, partial cds. | | |
| | LOCUS: | AF047431 1122 bp mRNA PRI Jul. 17, 1999 | | |
| | Effect: | ++ | | |
| | | Convention for evaluating the phenotypical ‘cell death effect’: | | |
| | | − negative, −/+ and −/++ weakly positive, + positive, ++ strongly positive, +++ very strongly positive | | |
| Clone # | T7 sequence | TGCTGGAAAGGGGAGATCCTGAGAACAGACCAGTGGCCTCGGAGGCTCTTCATGCAGCTCATTCCGCAGCAGTTGCT | | |
| 65 | | GACCACCCTCGTGCCACTGTTCCGGAATTCACGCCTGGTACAGTTCCACTTCACTAAGGACATGGAGACTCTGAAGAG | | |
| | | CCTTTGCCGGATCATGGACAATGGCTTCGCGGGCTGCGTGCACTTCTCCTACAAGGCATCGTGTGAGGTGCGTGTGC | | |
| | | TCATGCTCCTGTACTCCTCGGAGAAGAAGATCTTCATCGGCCTCATCCCGCACGACCAGAGCAACTTTGTCAACGGCA | | |
| | | TCCGTCGCGTCATCGCCAACCAGCAGCAGGTCCTGCAGCGAAGCCTGGAGCAGGAGCAGCAGCAGCGAGGGATGGG | | |
| | | TGGCTAGAGGATGCCTGGGCTGGGCGGGCCACAGTCCCAGACGAGGCCCCAGTGGAGACTGGTCA | | |
| | BGH sequence: | GCCNGCTTCTCNGCATTTAGGTGACACTATAGAATAGGGCCCTCTAGATGCATGCTGGAGCGGCCGCCCTTTTTTTTT | | |
| | | TTTTTTTTTTTTTTTGGGTTAAAAGGCTTTATTAGGGAAACATACAGGGGCAAGGACCATCCTTGGGAGACCTNAGG | | |
| | | ACGCTGTCCTCCAGGTTGCTGGGCAGGTACAGTCGCCAGGAGCCCCTGCTNAGAAGCAGCTGACCAGTCTCCACTGG | | |
| | | GGCCTCGTCTGGGACTGTGGCGCGCCCAGCCCAGGCATCCTCTAGCCACCCATCCCTCGCTGCTGCTGCTCCTGCTCC | | |
| | | AGGCTTCGCTGCAGGACCTGCTGCTGGTTGGCGATGACGCGACGGATGCCGTTGACAAAGTTGCTCTGGTCGNGCG | | |
| | | GGATGAGGCCGATGAAGATCTTCTTCTCCGAGGAGTACAGGAGCATGAGCACACGCACCTTACACGATGCCTTGTAG | | |
| | | GANAAGTGCACGCAGCCCGCGAAGCCNTTGTCCATGATCCGGCAAAGGCTNTTNAGAGTGTCCATGTCCTTAGTGAA | | |
| | | GTGGAACTGTACCAGGCGTGAATTNCGGAACAGNGGCACGAGGGTGGTCAGCAACTGCTGCGGAATGAGCTGCATG | | |
| | | AAGAGCCTTCGAGGCCACTGGTCTGTTCTCAGGATCTCCCCTTTCCAGCACACTGGCGGCCGGTAGTAGTGGATNCG | | |
| | | AGCTCGGTACCAAGCTTGGGTCTTC | | |
| | Serial clone | UI-3 | | |
| | number: | | | |
| | ACCESS No.: | U79287 | | |
| | Definition: | Human clone 23867 mRNA sequence 1396 bp | | |
| | LOCUS: | HSU79287 | | |
| | Effect: | ++(fast), strong laddering | | |
| Clone # | T7 sequence | ATCGNCCCCCTTGGTNNCCGGCTCGGACCCTNTCAACGCCCGCCAGTGTGCTGGAAAGCAGCGAGCGGCTGCTTCGT | | |
| 66 | | GGAGCAGAGAGGTGCATCACCAGGTTCCCGATGAACCCAGAGAACCCTCCACCGTATNCGNGCCCCGGGCCAACAGC | | |
| | | CCCATACCCACCTTATCCACAACAGCCAATGGGGCCAATGGGGCCTATGGGAGCCCCACCTCCTCAGGGGTACCCCTA | | |
| | | CCCACCACCTCAGGGGTACCCCTATCAAGGATACCCACAGTACGGCTGGCAGGGTGGACCTCAGGAGCCTCCTAAGA | | |
| | | CCACAGTGTATGTGGTGGAAGACCAAACGNAGAGACGACCTGCGCCCATCCACCTGCCTNANAGCCTGCTNGACTGCT | | |
| | | CTGNGTNGCTGCTGCCTCTGGGACATGCTNACCTGNTCANCTGATGAGCCCAGCTCTTCCGNTTGNCCGCTCTGNGCC | | |
| | | ANCTCCNATNCNTNCNCCTGNNCCCATCTCTCCTGCTTNCTCTNCAGNTGCCTANCCTCCTCCTCTCCACNCTCNTTTA | | |
| | BGH sequence: | CCTTTTTTTTTTTTTTTTTTTTCCAGATTTTGGACAGATTTATTGAAACATAAAGGGTATGAGCAGAGAGATCTAGTAGTGTGTCACATATTGC | | |
| | | CATTACCTTGAGTGTATAATTTAAACATTATAAATATATATTTCATAACTAAGCCTTTGGCCAAAAAAGTAAATTATTTAGCACATTTTTTAAA | | |
| | | GATCAGTAAGAAATGAGTTTTGAACATTAAAAAGATCAAGTCACTGAACTAAATAGCAGTAACCCTCACTYAATCTAAAACTCCATAGGACAGAA | | |
| | | AGTAGAGGTGTCTGCGCAGAGCTAGCCACTTTATAGCAATCAGAAGAGATGGGGCCAGGCACACTTATCGGAGGTGGCACAGAGCGGCCAAGCG | | |
| | | GAAGAGCTGGGCTCATCAGGTGATCAGGTGAGCATGTCCCAGAGGCAGCAGCAACACAGAGCAGTCCAGCAGGCTGTGAGGCAGGTGGATGGGC | | |
| | | CCAGGTCGTCTCTTCTTTGGTCTTCCACCACATACACTGTGGTCTTAGGAGGCTCCTGAGGTCCACCCTGCCAGCCGTACTGTGGGTATCCTTG | | |
| | | ATAGGGGTNCCCCTGAGGTNGNGGNAGGGGGTNC | | |
| | Serial clone | UI-5 | | |
| | number: | | | |
| | ACCESS No.: | C88489, AA073437, A1048028 | | |
| | Definition: | ESTs dbj/C88489, gb/AA073437, gb/A1048028 | | |
| | LOCUS: | | | |
| | Effect: | + (slow), weak laddering | | |
| Clone # | T7 sequence | CTGGAAAGGNGCGGAGCCTGCTGCTGCCATGGAGGCTGGNGGCTGGGTACACTNTGTCTAGACTGGGGGCCCTGGG | | |
| 67 | | CGGCTNNNGNGCCGGGTNGGGACNGTTACTTGGTACTGCCGCCGGCCTTGGATTCCTGTGCGTCCTTTACAGCCAGC | | |
| | | GATGGANACGGACCCAGCGCCNTGGCCGGAGTCACAGTCTGCCNAACTCCCTGGACTATGCGCAGGCTTCAGAGCGT | | |
| | | GGACGNCAGNNGACACAGNTTCGGGCTATCCCAGGTGAAGCTGNAGATGCTGCCATACTGCCCAGCCTCTCACAGGA | | |
| | | AGGGTANGANAAGGTGCTGGACCGCCTGGACTTTGCGCTGACCAGTCTTATGGCGCTGCNGCGCGAGGNGGAGGAG | | |
| | | CTTNAGAGAAGCCTGCAAGGACTAGCTGGNGAGATTGTCGNTGAGGACCGCTCTCATATATAAGAGAACCANANAGA | | |
| | | GGNCCNGGCGGCGCACGNTCCCTTTATGCCAGAGAGAGGAGNGACTCCACGGGCTNCAGCTCTGTCTACTTCACCGN | | |
| | | CTCCTCAGGGGCCNGCACTCACAGACGCCNANAGNNAGGGAGGCTATNCNNCAGCCCAACCGCGGAGTNTGATTACNA | | |
| | | GCNGNACTNCNACAAGGAANAGTGGANATGCNTGANGACCAAANNGAAGCTNN | | |
| | BGH sequence: | TTTGAAAGCCCGTTCTAGCATTTAGGTNCACTATAGAATTNTGACCTCTATATGCATGCTCGAGCGGCCGCCGACGGA | | |
| | | CTGGACGCCGCCTNCACATCCAGGTCCAGAGAGTCCTTCCTCCCCATCCTCACGGTCTCGCAGCTCACTTCGTCCTCA | | |
| | | GCATCTCCACTCTCCTTGTCGGAGTCCCGCTCGTAATCAGACTCCGCGTTGGCTGTTGTATAGCCTCCCTCGCTCTCG | | |
| | | GCGTCTGTGAGTGCGGCCCCTGAGGAGGCGGTGAAGTAGACAGAGCTGGAGCCCGTGGAGTCACTCCTCTCTCTGG | | |
| | | CAAAAGGGAACCTGCGCCGCCGGGCCACTCTCTGGTTCTCTTCTATATGAGAGCGGACCTCCCCGACAATCTCCCCAG | | |
| | | CTAGTCCTTGCAGGCTTCTCTGAAGCTCCTCCACCTCGCGCCGCAGCGCCATAAGACTGGTCAGCACAAAGTCCAGGC | | |
| | | GGTCCAGCACCTTCTCCTGCCCTTCCTGTGAGAGGCTGGGCAGTATGGCAGCATCTCCAGCTTCACCTGGGATAGCC | | |
| | | CGAAACTGTGTCACCTGGCGTCCACGCTCTGAAGCCTGCGCATAGTCCAGGGAGTTGGGCAGACTGTGACTCCGGCC | | |
| | | ATGGCGCTGGGTCCGTTTCCATCGCTGGCTGTAAAGGACGCACAGGAATCCAAGGCCGGCGGCAGTACCAAGTAA | | |
| | Serial clone | UI-6 | | |
| | number: | | | |
| | ACCESS No.: | AK001441 | | |
| | Definition: | homologous to Homo sapiens cDNA FLJ 10579 fis, clone NT2RP2003446 2251 bp mRNA Feb. 22, 2000 and ESTs, e.g. A1663355 and AU080732 | | |
| | Effect: | +++(fast), strong laddering | | |
| Clone # | T7 sequence | GTGTGCTGGAAAGCGCTGGGTCTGAGTGACCAAAGGCAGTAGCNCTCGCGGAGATCACCCGCTGNCCCTNGATCACC | | |
| 68 | | ATGTCGGCCTTCGACACTAACCCCTTCGCGGACCCAGTGGACGTAAACCCCTTCCAGGATCCCTCTGTGACCCAGCTG | | |
| | | ACCAATGCTCCTCAGAGTGGCCTGGCTGAGTTCAATCCCTTCTCAGAGACAAATGCAGCGACAACAGTTCCTGCCACA | | |
| | | CAAGCTCCTGGGCCCTCCCAGCCAGCAGTTCTCCAGCCCTCAGTGGAACCAGCACAGCCAACGCCCCAGGCTGTTGCA | | |
| | | GCTGCGGCCCAGGCAGGCTTGCTTCGACAGCAGGAAGAACTAGACAGGAAAGCTGCCGAGCTGGAACGCAAGGAGC | | |
| | | GAGAGTTGCAGAACACTGCAGCGAATTTGCATGTGCGAGACAACAACTGGCCGCCACTCCCCTCATGGNGCCCTGTG | | |
| | | AAACCCTGCTTCTATCAGGACTTCTCCACGGAGATCCCTGCTGACTACCAGCGGATTTGCAAGATGCTTTACTATCTC | | |
| | | TGGATGNTGCNTTCAGCGCTCTGTTTCTAAACCTNCTTGCGTNCCTGGCCTNTNT | | |
| | BGH sequence: | TTTTTTTTTTTTTTTTTNAAGTCCTTGTCGCTTCCTCTCCTTCCCTCTTGTAGAGGAGAAAGCTGACTACTGAGGGAG | | |
| | | GAGCAGGGACAAGACAAATCCGTGCCAGGTCTGTGCTGGTGTAGTCACTGCATAAGGCACCCGAGGAAAGTGCAGCT | | |
| | | CAGAAGACAGTTGACAGAGAGGCCAGGGCTCAGGGAAGGAGAGTCGGGAAGCCTAATTCCCCTGAAAGGCTCCTCG | | |
| | | GGCAGCAGATGAGGCAGCCCCGCGAAAGGTTCTGCTGCTGAAGATACCCTGGGAGAACTCTTCCTGGGGCTGCTGGA | | |
| | | AGCTGGCTCCTGTTCGGCGGTAGAAGGCGTGTACCCGCTGCAGGAGGAAGAGTGAAAGCCCAGCACAGAGGGTAAA | | |
| | | GAAGCCAGCCACTACCATCATGATGATGGTCACAGCTAAGGGCCCATTTTTCATCGTAGATAGGGCTGCAAGCCAACC | | |
| | | ACTGGTCCCCAAGTTAGGCAAGCCAATCAACTGGATGAAGTAGATCCCTATTTGACAAAAAAATACAAAAAAGAACAC | | |
| | | GAAGAAGCTGAAAGAGTTGTCAGACCTGAAAGGCTTGTAGATGGGTCGGTCCAAC | | |
| | Serial clone | UI-8 | | |
| | number: | | | |
| | ACCESS No.: | AF005038 | | |
| | Definition: | Homo sapiens Secretory Carrier Membrane Protein (SCAMP2) mRNA | | |
| | LOCUS: | | | |
| | Effect: | −/+ | | |
| Clone # | T7 sequence | GTGTGCTGGAAAGGCAGCATCTATTCGTCACTGCCAGGCTGAACTGGAGAGGCTTTCAGAGTCCCTGAGGAAGAGCA | | |
| 69 | | TCTCCTGTGGAGCTTTCTCTGTTCCTGGGGGTCACAGCCTCTACTTAGAAGCCAGGAAGAAGATTGAGCTGGGCTAC | | |
| | | CAGCAAGTGCCGAGGAAGGGAGTGAAGGCAAAAGAGGTTCTCAAGAGTTTCCTACAGTCACAGGCTATTATGGAGGA | | |
| | | CTCTATCTTGCAGTCAGACAAAGCTCTTACAGATGGAGAGAGAGCCATAGCAGCTGAGCGGACAAAGAAGGAAGTGG | | |
| | | CTGAGAAGGAACTAGAGCTGCTGAGGCAGAGACAGAAGGAGCAAGAGCAAGTGATGGAGGCTCAGGAGAGAAGCTT | | |
| | | CCGAGAAAACATTGCTAAACTTCAGGAGAAGATGGAGAGCGAAAAGGAGATGCTGCTGAGGGAGCAGGAGAAGATG | | |
| | | CTGGAGCACAAGCTGAAGGTCCAAGAAGAACTGCTTATTGAAGGATTCAGAGAGAAATCTGATATGTTAAAGAATGA | | |
| | | AATAAGTCACCTGAGAGAAGAGATGGAAAGAACAAGAAGGAAACCCTCACTGTTTGGTCAAATCCTTGACACCCGTTG | | |
| | | GCAATGCGTTCATTATGATTTTACCAGGAGCTGGTAAACTATTTGGTGTGGGGCTGAAATTCCTCGGCTCACTAAGT | | |
| | | AGTTAGTCTAAAGAGTTTCTGGGAAGGAATTTATAAAATCCAGCTTCTGGTATTTTGATACCAAACTTTTTGCTGGGA | | |
| | | TTTTTAAGTNCACATTCTAGTTTAATAACCACNGTTACCAAGAAAGTAAGACTTTCATT | | |
| | BGH sequence: | TTTTTTTTTTTTTTTTTTTTTTTTTTTTTTTTTTTTTTATTTTAATCTGCATGCCTTTTATTATCAATCAAACATTTTCC | | |
| | | TCATACATTCTNTTATGAAAACCCACTTGNGCGTTTTAACAAGAGCTCTTTTTAACAATTTATCTGTAGTGTACAACTG | | |
| | | GGATACATATGCCAATGNGTCTTCATTTGGCCATGTTTCTGNTTGATTTACTTACATCTTTTCTAGAAATAATTTCACT | | |
| | | ATATTCTGACTGGCCTCCCTCTTTATTATTTAGATAACATCACTGAAATTTGACAGGATTTATGGACGCCAGACCCTTA | | |
| | | TATTCTTCAATCATTGGTTTGNATCTTTAACCTTAAATATAATAGGNAATGAACTGACACAGAATTAGACTCCGAGGAA | | |
| | | GCAGTATCTGACAATTTAGCTATTAATGGAAGTTCTTACTTTCTTTGTAACCGNGGTTATTAAACTAGAATGNGTACT | | |
| | | TAAAAATCACAGCAAAAAGTTTGGTATCAAAATAACAGAAGCCTGGATTTTATAAATTCCTTCCCAGAAACTCTTTAGA | | |
| | | CTAACTACTTAGTGAGCCGAGGAATTTCAGCCCCACACC | | |
| | Serial clone | UI-10 | | |
| | number: | | | |
| | ACCESS No.: | U44731 | | |
| | Definition: | Mus musculus putative purine nucleotide binding protein mRNA, | | |
| | LOCUS: | MMU44731 | | |
| | Effect: | −/+ | | |
| Clone # | T7 sequence | TCGCACCANCCTTGGACCGAGCTCGGATTCCCTAGTAACGGCCGCCAGTGTGCTGGAAAGAACGCCTCTGGGGAGCA | | |
| 70 | | TGGCATCGTGGTTTTCTCTTTGGGATCCATGGTCTCAGAGATTCCGGAGAAGAAAGCCATGGAAATTGCTGAGGCTT | | |
| | | TGGGCAGAATTCCTCAGACGGTCCTGTGGCGCTACACCGGAACTAGACCATCGAATCTTGCAAAGAACACAATTCTTG | | |
| | | TCAAATGGCTACCCCAAAATGATCTGCTTGGTCATCCAAAGACTCGGGCATTCATCACACACTCTGGCTCCCATGGTA | | |
| | | TTTATGAAGGAATATGCAATGGAGTTCCGATGGTGATGATGCCCCTATTTGGCGATCAGATGGACAATGCCAAGCGC | | |
| | | ATGGAAACTCGGGGAGCTGGGGTGACCCTGAATGTCCTTGAAATGACTGCTGATGATTTGGAAAATGCCCTTAAAAC | | |
| | | TGTCATCAACAACAAGAGCTACAAGGAGAACATCATGCGCCTCTCCAGCCTTCACAAGGACCGTCCTATAGAGCCTCT | | |
| | | GGACCTGGCTGTGTTCTGGGTGGAATACGTGATGAGGCACAAGGGGGCACCACACCTGCGCCCGGGCGCCCATGAC | | |
| | | CTCACCTGGTATCAGTACCACTCCTTGGATGTGATTGGCTTCCTCCTGGCCATTGTGTTGACAGTGGTCTTCATTGNC | | |
| | | TTTAAATGTTGTGCCTATGGCTGCCGGAAATGCTTTGGGGGAAAGGGGCNAGTGAAGAAATCACACAAATCCAAGAC | | |
| | | CCATTGAGAAGTGGGGGGAAGTGAANGAGAAGTATTAGTTCATTATCTGACAGTTGAACTTTGGNAACAAGTGTTNG | | |
| | | ANCCATAATGGTTTGTTAGGGGAA | | |
| | BGH sequence: | TTTTTTTTTTAGTGATACAATTTATATGAATTTATTGATAAGTTCTTGGTTTGGGAACACAATAGAAGATGTACTTGC | | |
| | | CTTAGAACATACTTTGGTTTTCATCAAATTCCGGCACAAGCAACAATTATTATCTCAAACACAGGGCCATCAGTGTCAC | | |
| | | AGCCTCATCTCTTCTCCCAAAGCTCCAAAGGTCTTGCTACCCATAATGCATGAACACAATGAAGAATGACTAACACTGG | | |
| | | AGAAGGCCACGGCAGTCTGAGAATCCAGGGGAGAAAACCATCAGCTGGCGGGAGCAGGGGAAACTGTGGGGCTGTC | | |
| | | ACTGAAAGCAGAGTGCCAGACTTTCTGTAAATTACCAACAGATGGCAAGAGTTGTCTAGACTGAAGATACTGGAAAAG | | |
| | | CACATCACATATACTAGAACCTTCCCTGATGGGTGTGGCCAGCAATTAGATTGTTATTATTATTATTTTTAAATGTGC | | |
| | | TGAATGTATAATGTATGGTGAATTATTTCCCTAACAAAAACAATATGGATCTAACACTTGTTTCCAAGTTTCAACTGAT | | |
| | | CAGATAATGAACTAATACTTCTCCTTCACTTCCCCCCACTT | | |
| | Serial clone | UI-12 | | |
| | number: | | | |
| | ACCESS No.: | U16818 | | |
| | Definition: | Mus musculus UDP glucuronosyltransferase (UGTI-06) mRNA | | |
| | LOCUS: | MMU16818 | | |
| | Effect: | −/+ | | |
| Clone # | T7 sequence | GCCAGTGTGCTGGAAAGAGAAGCAGACATGGCTCAATTTGCACAGGTCATGGCTGAAGTGGGCGACTTTGGTCGCTT | | |
| 71 | | TCAGGTGCGGTTGACCATCCTGATGGGCATTCCCAATTTCCTGGCTGCATTCTTCATATTTGGCCAAGTCTTCATGGT | | |
| | | CCTTGATGAGGCTCACCACTGTTCAGTGTCCTGGGTTAAGAACCACACTTTCAACCTAAGTGCCGCTGAGCAGCTGGC | | |
| | | TATAAGCATCCCCAACGACACAGCGGGCAGACCCGAGTCCTGCCTCATGTTCCGGCCACCTCCTGACAGTGCCAGCCT | | |
| | | GGAAGACATCCTGAGCCACCGCTTCAATGAGACACAGGCCTGTGACTCAGGCTGGGACTATCCTGAGAACCGGCCTC | | |
| | | AGTCCCTAAAGAAAGAGTTTGACCTGGTGTGTGATCGAAAGAACCTGAAGAAGACCTCCCAGTCGGTGTTCATGGCT | | |
| | | GGTCTCCTTGTTGGGGCCCTGGTCTTTGGGCCTGTCTGTGACTGGATTGGCCGCAGACCCTCCCTCCTGATGCAGGT | | |
| | | GCTTCTGTCAGGCATCACAAGCATGGCCACAGCCTTCGTGTCCAGCTTTGAGCTCTACTTGGCCCTACGCTTTGTCTT | | |
| | | GGCTACTGCCAATGCTGGATTTTTACTAAGTACCAATGTCCTGATTTCANAGTGGGTGGGGCCATCTTGGAGAACAC | | |
| | | AAGCCGTGGNCTTTGGCCCANAGCAACGTTGCCCTTGGGCAGATGGTGTTAGCAGGACTGGCCTATGGTGTCCGAAA | | |
| | | CTTGGAGACTTCTTCAGATACANGGACCCGACCCGCTTACTGNTCTTCTTCTATTTCT | | |
| | BGH sequence: | TTTTTTTTTTNTNGATATTAGNTANGTTTTATTATTTNTTATCTNTATGAGGAAGGGGTATCCCAGACAGGGAGACTG | | |
| | | NTGAGGNNACAATCCTAGAGAGANTGTCNGAGAAGACTGANAGCGACCCTGACTCANCCGTGGTACTGGGGTGCAGT | | |
| | | TNGTGTATGANAAGTGTGGAANAGGGAAGGGTACTGTCCCTGATACTCCCTCTGGGCTCTGCAAAAGGGAGTAGGGA | | |
| | | GCATGAACACGGCTGATCTGATCCTGTGNGCTANAGATGCCATGTGCGCTGCCTCTGCTCTTGATAGGCTGGNCCTC | | |
| | | ANCAGCCACAGTCANAANTNCGANCTCTTCACAACANCNACNCTCANACTNNAAGATCTTCCCAAAGCCTNCANGTCTT | | |
| | | GNCTNNGNNNTGCNGATNNGAGGGACCCTGCAAGGAGCCCCTGNTCNAGGANCTGGAGGNTGTNCTNCAGGGTCNGG | | |
| | | CCCTGGGTCTCAGGAAGTAGAGCGNACAGTANGCCTGCTNCAATGGNGAGGCTGCNAAAGATGACNATGGGAATCGN | | |
| | | CTGGTGGACTGTTCTAGTAGCATCACAANGGGTGTGATGATGCCACCGACCCTGTAGAAGATGCTNACCAACCCCAT | | |
| | | GCCTGTTTGCCTGATNATGGATGGTNGANGAGCTNAGCTAGTGNACACATANGAGATGGTGAANGCCGCAACCGAGN | | |
| | | CAANATTTCNCNATCATAGGCAGNANANAGGCNCACCGNGGGANGAACTCCTGGGATGNAAAATATAATGCCGNCCAT | | |
| | | GANCCCGNCTGGGACNGCANTCNACAGNTTGTTTTCNNA | | |
| | Serial clone | UI-14 | | |
| | number: | | | |
| | ACCESS No.: | NM_004256 | | |
| | Definition: | Homo sapiens organic cationic transporter-like 3 (ORCTL3) mRNA. | | |
| | LOCUS: | | | |
| | Effect: | −/++ | | |
| Clone # | T7 sequence | GGAAAGGGAGCAGCAAACGGCCGGCGGCAGGCGCCGCGGGGGGGGCGGGCGGCGCGGAGGCGGCGGTGGCCATG | | |
| 72 | | GCCGAGGCGTCGCCGCAGCCCGGACGGTACTTCTGCCACTGCTGCTCGGTAGAGATCGTGCCGCGCCTGCCGGATTA | | |
| | | CATCTGCCCAAGGTGCGAGTCTGGCTTCATTGAGGAGCTTCCAGAAGAGACCAGGAACACAGAGAATGGCTCAGCCC | | |
| | | CCTCCACAGCCCCCACCGACCAGAACCGGCAGCCATTTGAGAATGTGGACCAGCACCTGTTCACGCTGCCACAGGGAT | | |
| | | ACAGCCAGTTTGCCTTTGGCATCTTCGACGATAGCTTTGAGATTCCCACGTTCCCTCCTGGAGCCCAGGCGGATGATG | | |
| | | GCAGGGACCCTGAGAGCCGACGGGAGAGAGAGCACCAGTCTCGGCATCGGTACGGGGCCCGGCAGCCCCGTGCCCG | | |
| | | CCTCACTGCCCGCCGGGCCACTGGCCGGCATGAAGGTGTCCCTACGCTGGAAGGGATCATCCAGCAGCTCGTGAATG | | |
| | | GCATCATCTCTCCGGCTGCTGTGCCCAGCCTGGGCCTTGGTCCCTGGGGCGTCCTGCACTCGAACCCAATGGACTAC | | |
| | | GCCTGGGGGGCCAACGGCCTGGACACCATCATCACGCCAGCTCCTTAATCNGTTTGAGAACACCCGGNGCCCCCACTT | | |
| | | GCAGACAANGAGAAGAATTCAGGCTNTTCCCCNCGGTGCCAGTCACA | | |
| | BGH sequence: | TTTTTTTTTTTCTGCTTTTTTGAGTAGGNGTATTTTAAATAGCTTTCCAGATAACACACATTTCCCTTAAAAAAAAAA | | |
| | | ATTGTCTGCTGGAGCAGTTTGTTCCTGGGTCTGCTGGTCACCCTTGNGGTCCCGAGGCCGGCGCTGGTTGGGNGCT | | |
| | | GGGCCCGGCCTCCCAGGGCGTGTGGCGGGCTGGGCCTGGCGACAGTTCTGCTGANTCTCCACAGACCATGCATGGC | | |
| | | CGNACGTGGCTCAAACGTCCATTTATTTCAAAGCAGTAATAATTTAAAATCATAAAAACCCTTCTGCCGTTGAACATTT | | |
| | | GGAGGGTGAGGTTAANACGGACTGAAGGTGCCTGCCTGCTGGGGACATGGGACCCACGTGCACACCTCCAAACAGG | | |
| | | AGCGGCCATGGCCAAGTCCAAGCCTGCCCGNAGCCTGGGGCCCGCCAGGNACTGCCGGCCGCCACGGGCAGCACCTC | | |
| | | TAGTCCACAGCCATGGCGGGCGGGCAGGCGGNCGGTGCTGGGGCACCTGGGGTGGGTGGGATGACCAAACCCCGG | | |
| | | GGGGGATGGNCAGGTGGGACTCAGGAGTTTGCTTGNGGGCGTTCTCATTGCTGGGCNAAACTGGAGGAGGATGAAN | | |
| | | GACCAANGACTAANGAGGAAACCTACACCGGGCAAAGCCTTGGGGGGAATGGNCGGCTTGGGTNCTGTCCNAGTGAG | | |
| | | GCTNTTTACGGCAAANCCGGGCAAGCNTGGCATGCTGNNTTCAGCCANGGGCACGAATCCNCNTGTCGTGGNAAAAG | | |
| | | GTGGGNTGCAAGGGCNAANTTNGCCGGNANACTTTTTACCCAGGGGGAATAAANNCTTNTTTTAACNNACTGGGNCAA | | |
| | | CTTAAAGCCCTTGGAGCCCCNCGNTGGTTCCTNCTGGGGNCTTNGGGACCCNTNNNGGAANAAACCTTGAAANTTCTC | | |
| | | CCTTGNNNTGCANCTTGGGGGGCCCCNGGGTTTTCNAANCGGAATGNGGGACTTTCNATNAAANAAGGGGNNCCAGC | | |
| | | CNTTGCCCCCCAGGCGGAACCCANTNGGNTTCAAGTCCGGAACCCCCNNGGGCCAAGGCCCANNTGNCCAAAAACCG | | |
| | | GGAAAAAAGCNTTNCAAAATNTNGGAGAACCTTCCCGCNGGCCCCTTTTTNCCCAAGGNCCCNGGCAANGNGCACCGN | | |
| | | TNTTNGGCCCTCCCAACCAANNGGTTTTTCTCGNGTNAGGCTTNNTAAANNCGGCTNCCGGNAANGGGAAANAAANNN | | |
| | | TAAACCNCGCNANGTTTTTNTTNNC | | |
| | Serial clone | UI-15 | | |
| | number: | | | |
| | ACCESS No.: | AK000559 | | |
| | Definition: | Homo sapiens cDNA FLJ20552 fis, clone KAT11732. | | |
| | Effect: | + (moderately fast), no laddering (? ) | | |
| Clone # | T7 sequence | AGTGTGCTGGAAAGCGGGCCTGAGCCGAGCAGCTGCGCGACGTCATGGACAACTCCGGGAAGCAGGCTGAGGCTAT | | |
| 73 | | GGCGCTGCTGGCTGAAGCGGAGCGCAAGGTGAAGAACTCGCAGTCCTTCTTCTCCGGCCTCTTTGGAGGCTCATCCA | | |
| | | AAATAGAGGAAGCATGCGAGATCTATGCCAGAGCGGCGAACATGTTCAAGATGGCCAAGAACTGGAGCGCTGCTGGG | | |
| | | AACGCTTTCTGCCAGGCTGCCCAACTACACCTACAGCTCCAAAGCAAGCACGATGCAGGCACCTGCTTTGNGGACGCT | | |
| | | GGCAATGCTTTNAAGAAAGCTGACCCCCAAGAGGCCATTAACTGTCTGATGAGAGCAATTGAGATCTATACAGACATG | | |
| | | GGCAGATTNACAATCGCAGCCAAGCACCANATCTCCATGGCTGAGATCTATGAGACAGAACTGGTGGATGTAGAGAA | | |
| | | GGCCATCGNCCACTATGAGCAATCTGCAGACTACTACAAAGGANAAGAGTCCAACAGCTNANCCAACAAGTGTCTGCT | | |
| | | NAAGGNGGCTNCTACNCCNNACAGCTGGAGCAGNCCCANAAGGCTATCNA | | |
| | BGH sequence: | TTTTTTTTTTTTTTTTTCATGCAAAAGTGGCAGGTTTNATTGTCCTTTTTGGGCCAGCTGNAGCTTNAGGTCGATAGA | | |
| | | CCTGGATGCATGGAGAGAAGCAGGTGCGCAGGGCAGGGCCAAGGCACCGCATGAGCTCCAGGGCTGGGTCTAACCT | | |
| | | GAGAGGTTGGCATTGNAGGAACAAGGGAAGGCTCCAGGGGCAGAGGTTACCCCCAGCCAGGGAGAGNAAGTNTCAG | | |
| | | AGCTACCNGNGTNCTTATCTCTGCTAGGGGACAAGGTAGGAGATATGGAAACAGNCTTAGTACNCTGCTGNTNAGGA | | |
| | | GATGNTNCCANNCCTTGANCTNCGGTACNTNAAGCACACAGANT | | |
| | Serial clone | UI-18 | | |
| | number: | | | |
| | ACCESS No.: | X89968 | | |
| | Definition: | Rattus norvegicus mRNA for alpha-soluble NSF attachment protein | | |
| | LOCUS: | RNSNAPGEN | | |
| | Comment: | Effect: ++, large phenotype, no laddering | | |
| Clone # | T7 sequence | AAAGTTGTTCTTCTCGTGGTTCCCAGTGGCGAGAGGAGGAGGAAGCCCNGAGCGGAGCGGGGCGGCTGGGGGGGG | | |
| 74 | | TGGACCCGCCGCGGCTGCTGCTGCCACCGGCGGCGCCGCCACCACCGCTCGTGGGGCTCGTGGCGTGAGGAAGGAG | | |
| | | GACGAGTGAGACCCCGGGGCGAGCGGGCGGCGGCGGCGCTGCTGCTGCTGCTGCTGGGGGAGGGNCGGCGGCGGG | | |
| | | ACGGCGATGGCGGATATCGACAAACTCAACATCGACAGCATCATCCAACGGCTGCTGGAAGTGAGAGGGTCCAAGCC | | |
| | | AGGCAAGAATGTCCAGCTCCAGGAGAAGGAGATGCGAGGACTCTGCCTGAAGTCTCGGGAGATCTTCCTCAGTCAGC | | |
| | | CTATCCTTTTAGAACTTGAAGCACCACTCAAGATATGTGGNGACATCCACGGGCAGTACTATGATTTGCTCCGTCTGT | | |
| | | TTGAATACGGTGGCTTTCCTCCAGAGAGCAACTATTTGNTTCTCGGGGACTATGTGGACAGGGGCAAGCAGTCCCTG | | |
| | | GAGACAATCTGNCTCTTGCTGGCTACAAAATCAAGTATCCGGAGAACTTCTTTCTTCTCAGAGGGAACCACGAGTGCG | | |
| | | CCAGCATCAATAGGATCTACCGATTTTATGATGAGTGTAAAAGAAGATNCACCATTAAGCTTGTGGNAAACGTTCACA | | |
| | | GACTGGTTTAACTGCTTGCCGATAGCAAGCCATCNTGGACNAAGAAGANAATCTGCTGTCATGGAGGTTATCACCNGA | | |
| | | ACTTCAATCTATGGAGCNGAATTCCGCGGAAATATTAGACCANTTGANGTACCNGAACAAGGGCCTTCTTTGGGAACC | | |
| | | NTTGGGGGCCTGANCCCCATAAAGAGGCNTTAAGCTTGGGTGGAAAATGCCCGAGGAGNGGCCTTCCAATTGGTGCC | | |
| | | AAAT | | |
| | BGH sequence: | TTTTTTTTTTAACAAGCTTGCATTTAATAAGTCTGAAACCATTCTCAGCACATGGCATTGTACACGGGCATCTGTGCA | | |
| | | AACAGATTCATTTAACAGGTCGTAGTTTAAAAAAGTCATAGATAGTGTGAGTTCTGTATAAACCGGTGGACGGCAAGT | | |
| | | TAGTTCCTTTTGATTTATAAGCCTCAATGTCACCGGAGAATAAAGAATGTAGCCAAAGAAAGCATTATCGGTCACTCG | | |
| | | TATAGGACAGTGTTGTTTCTATAATTTGAAGCTTTCTGAATGGACGGGTTCAGGCCTGATCCAAGCCGTGGTGGNGT | | |
| | | GACAGGTCTCGTGGCGNTGGGCTTCTTTTTCTCTGCAGGCTTTAAAATCTGGAAGGAACACATGAGGGTCTCATCCA | | |
| | | CACTGATCATGGCGCCTGCATTGTCAAACTCGCCACAGTAGTNGGGTGCAGAAAACAGAGTGACTAACTGCCTCTTTG | | |
| | | CAAAAAACTCATAGCCATCTTCAACCACCTGATGGGCTCTACATATAAGATCCAAATCATGCTTATGGAGAAATTTTGC | | |
| | | AACCACTTCTGCACCAAATGTGAAAGGACACTTCTTCTGNCAATTTTCACCCCAGNCTAAGACATCTTTTAATCGGGGT | | |
| | | CAGGACCNCCAAAGGATCCCAAAGGAAGAACTTTGATCTGGTACATCAAGTTGGGCCTCANAAATTCGNCCGGAANCT | | |
| | | GGNTTCCATAGAATTGAAGAACTGGGGGAANAAACCTTCCATGACAGGCNGAAAAATCTTNTTNGTCCCCCCAANGGC | | |
| | | TTGNTAATTGGGCAAGGCNGGTTNAAAACAGTCNTTGTGAACCGTTTTTCCNGAGCTTNAAAGGGTGGGAAACTTCTT | | |
| | | TTTCCCCTTCATCATTAAAAAAACCCNAGGAAACCTAATGGAAGCTTGGGCGCCAAATCGGGGGTNCCCTTTTTGGGA | | |
| | | AAAAAAGGAAGNTTTTTCCGGGAAACCTTTGGATTTTTTAGGGNCCCCCCAAGNNGGGCAAAATTGGNTTNCCNGGG | | |
| | | NNCT | | |
| | Serial clone | UI-20 | | |
| | number: | | | |
| | ACCESS No.: | D85137 | | |
| | Definition: | Mouse mRNA for PP1gamma (protein phosphatase1gamma) | | |
| | LOCUS: | MUSPIM1K | | |
| | Effect: | −/+ | | |
| Clone # | T7 sequence | GCCGCCAGTGTGCTGGAAAGNGAGACTCGGCGATACTGCACTTCCTCAGAGCTTGCTGCCCACTACTGCAAGAAGTA | | |
| 75 | | CAACATCCAGTACCAGGAGAGCTTCTATGCCTGGAGCACCCCAGGCGTGGGCAAGTTNGTGACTTCCATGGCTGCCTGCCT | | |
| | | CAGGGGGCATCTATCTCACCCTGCTGTTCCTCATTGAGACCAACCTGCTGNGGCGACTGAGAACCTTCATCTGTGCCT | | |
| | | TCCGGAGGAGGNGGACTCTGGCAGAACTGCAGAACCGGACATCAGAGCTGCCCGAGGACCANGATGTAGCTGANGA | | |
| | | GAGGAGCCGAATCCTGGTCCCTAGCTTGGACTCCATGCTCGACACACCACTGATTATCAACGAGCTCTCCAAGGNGTA | | |
| | | TGACCAGCGAGCACCGCTCCTTGCCGNGGACAGGATCTCCCTTGCGGTCCAGAAAGGGGAGTGCTTCGGCCTGTTGG | | |
| | | GTTTCAATGGAGCTGGAAAAACCACAACATTCAAAATGCTGACTGGGGAGGAGACCATCACCTCAGGGACGCCTTT | | |
| | | GTTGGTGGTTACAGCATCAGTTCTGACATTGGGAAGGTGCGGCAGCGGATGGGCTACTGCCCCCAGTTTGATGCACT | | |
| | | GCTTGATCACATGACTGGCAGGGAGATGCTGGTTATGTATGCACGGCTCCGAGGCATCCCANAGCGGCTCATATCAATG | | |
| | | CCTGTGTGGAGAATACTCTGCGGGGTCTGCTGCTGGAACCCGCACGCCAACAAACTAGTCAAGACTTACAGTGGTGG | | |
| | | TAACAAACGCAGCTAAGCACTGGCATTGCCTCATTGGAAAGCCTGCNGTTATCTTCTGGA | | |
| | BGH sequence: | TTTTTTTTCATGTCCCCTTATCCTTTATTTTTNATANAATCACAATATGTTTAAGNGTATCCTCAANANATGACACTACT | | |
| | | CAGCCCTGNANNGGTCCCTGACAATCCTGNGGCCAGNGACTTNTTGAAGGCACANAGCTAGGAACAGAGGATGNGCT | | |
| | | TCAAGTCTGGCCACAGCTCTCTCTTGNGCTGCCGATGCTCTGNGGGATGACATCTGGCTCAGGATCAGATCAGCCGG | | |
| | | GTCCACTGTCCATACTGGAAGGAACATTGACATCCAAGGATGCCTNTGCAGTTGCATTGGGAGCCAACGGTCTCAGC | | |
| | | ACCTAGTCATAGCCAGTATCTTCCCTTCATGAAGGNATTTGCAACTTTGCTGCTCATTCCTGGCAGCCTGGAAGTCCT | | |
| | | CATTGCTGCTNAACCCTGTNGGCTTTCTTCCCCCCCCCCCNAGGGGAGCTTAGNGTCATATGTAAACATAGAGAGGTC | | |
| | | CCTGGGGNCTCTCTGGATAANCACTGGAAAGAAGCTCAATCCGCCAGGCTAGCTGCAGAACCAGCTGGGACAGTTCC | | |
| | | CCATCCAAAGACTAAGGCCTGTCTGTGTGCACAGCAGGATGGGGNCTGGGACTGNGGTCCAGANACCTGACTTCAGG | | |
| | | AGTGGAAACCTGCATGCTGAGTCCTGCCTGCAGGCACTGACAGACACACATGGTCCACTTTGNGCATATCACCTTGAN | | |
| | | TGGTTTTCATGNCCATCATAGAAAAGAGTGATTAAAAATGANNGAAAGGAGAAAACTGGGAGGGAAAGGACCCACGG | | |
| | | GNTAAGCNCGAAATNGCCCCTTNCACGCCCNGGGG | | |
| | Serial clone | UI-25 | | |
| | number: | | | |
| | ACCESS No.: | NM_001089 | | |
| | Definition: | Homo sapiens ATP-binding cassette, sub-family A (ABC1), member 3 (ABCA3) mRNA | | |
| | LOCUS: | | | |
| | Effect: | + | | |
| Clone # | T7 sequence | CGNCACCCCCTTGGTACCGAGCTCGGATCCCTAGTAACGGCCGCCAGTGTGCTGGAAAGGATCATCCGTCTCGCCTC | | |
| 76 | | CAGTTCTTCACCGGCTGAGCTGGGTACGGACATCTCAGTGACCACGAGCTAACATAGCACGTGAAACTGACCTCAGG | | |
| | | AAGAACGAAGCCTGGAGACCCGGAGTGCGACAGCGACCTCCGGACTGAAGTGGAAGAAGCGTAGCGAGGCCTTCGT | | |
| | | GTCCTCGCTGTGGGGGGCGGGGCTTGTTGTGCAGGCGGGGCTCACGTGGTACTTTGGTACTTTGGATGACTGAAGG | | |
| | | GAAGCTTGGTACCCGCCTGGTCACTACCCGGAAGTCCAGCCGGAGTAGCACTACCCTGGAGTCCACCCGGCTGTGAA | | |
| | | GCGGNGGTGCTCTTAGGGGTGCTTGACGCCCTCATTGTTAGAAACTGATGGGTACTGTTGGCCGATTCCTAGACCCG | | |
| | | CTTTTGACCTACGTGATAAATGTGTTTCCGGAACTCTGCCAAAAAGTGTTCTGGAGGTTTTTGTTTGTTTCGGAGCCA | | |
| | | AGGCCTCCCTCGTAGCTCTTAATGGCCTGAAACACTCTTTGTAGCCTAGACTGGCATTGAAGTCAAGTTAGCCCTGCC | | |
| | | TTTGCCTCCCTGGGCTCTGGGATTACCCTGCCTTTCTTGATTATTTCTATTTGAACAAATATGAACAAACTATGTGGC | | |
| | | CACCACAAA | | |
| | BGH sequence: | TTTTTTTTTTTTGTGGNGGCCACATAGTTTGTTCATATTTGTTCAAATAGAAATAATCAAGAAAGGCAGGGTAATCCC | | |
| | | AGAGCCCANGGAGGCAAAGGCAGGGCTAACTTGACTTCAATGCCAGTCTAGGCTACAAAGAGTGTTTNAGGCCATTA | | |
| | | AGAGCTACGAGGGAGGCCTTGGCTCCGAAACAAACAAAAACCTTCAGAACACTTTTTGGCAGAGTTCCGGAAACACAT | | |
| | | TTATCACGTAGGTCAAAAGCGGGTCTAGGAATCGGCCAACAGTACCCATCAGTTTCTAACAATGAGGGCGTNAAGCAC | | |
| | | CCCTAAGAGCACCACCGCTTCACAGCCGGGTGGACTCCAGGGTAGTGCTACTCCGGCTGGACTTCCGGGTAGTGACC | | |
| | | AGGCGGGTACCAAGCTTCCCTTCAGTCATCCAAAGTACCAAAGTACCACGTGAGCCCCGCCTGCACAACAAGCCCCGC | | |
| | | CCCCCACAGCGAGGACACGAAGGCCTCGCTACGCTTCTTCCACTTCAGTCCGGAGGTCGCTGTCGCACTCCGGGTCT | | |
| | | CCAGGCTTCGTTCTTCCTGAGGTCAGTTTCACGTGCTATGTTAGCTCGNGGNCACTGAGAT | | |
| | Serial clone | UI-26 | | |
| | number: | | | |
| | ACCESS No.: | ESTs AI315969, AI930239, AI666299, AV141103 | | |
| | Definition: | mouse ESTs | | |
| | LOCUS: | | | |
| | Effect: | −+ | | |
| Clone # | T7 sequence | TTCACCCCGCTTGGTACCGAGCTCGGATCCCTAGTAACGGCCGCCAGTGTGCTGGAAAGATGGAGCCTGCGTTTCAC | | |
| 77 | | AGAGGGGATCTCCTTTTCCTCACGAACCGAGTTGAAGATCCTATACGCGTGGGGGAGATCGTTGTTTTCAGGATAGA | | |
| | | AGGAAGAGAGATTCCTATAGTGCACCGAGTCCTGAAGATCCATGAAAAGCAAGATGGGCATATCAAGTTTTTAACCAA | | |
| | | AGGAGATAATAATGCTGTTGATGACCGAGGTCTCTATAAACAAGGACAACACTGGCTGGAGAAGAAAGATGTTGTGG | | |
| | | GGAGAGCAAGAGGGTTTGTTCCTTACATTGGAATTGTGACGATCCTCATGAATGACTATCCTAAATTTAAGTATGCAG | | |
| | | TACTGTTTCTGCTCGGTTTATTTGTGCTGGTCCATCGTGAGTAAGAAGTCGGACTCCCTGTTCCTAGGAAGCTGCTG | | |
| | | TGCTTGTTGTTACTGAATGTTGGAGTAGATCCTGATCTGTGATTGCGGATTTTCGGAGGACACACACGTTGGCACTT | | |
| | | CTTGGTAGCCCTGGTTTGCATTGCTTTGTGTTTCCACACCAGAGGCTGTGTGGGCGGGTGCATGTGCACCGTGGAG | | |
| | | TGCACACAAGGGGACTGTCAATCACAGGGTTTCATATGTTGTCATTGTCACTCTTTCACATTTTTGTCATCAGTGAAT | | |
| | | TTTTTATATTAAAAGGTTGAGCCAAAGCCCCCAGTGTTTGTATTTTGAAGCCNAGCTTCACTTTAAAAGTGCCTACAG | | |
| | | AGTTCTGTAAATGAAAACACAGCTCTGCATGAGTTCAAACCTGNCGGTCCTTCTTACAGTAGGAATGGCNCATANTGA | | |
| | | GGCGGGCATAAGTCTTACTTT | | |
| | BGH sequence: | TTTTTTTTTTTCAATGNGCAAAGTCTTTTATTTAAAATTTTGAAAAGTTAANACTTATGACCGCCTCAATATGNGCCAT | | |
| | | TCCTACTGTAAGGAGGAACGACAGGTTTGAACTCATGCANAGCTGNGTTTTCATTTACAGAACTNTGTAGGCNCTTTT | | |
| | | AAAGNGAAGCTTGGNTTCAAAATACAAACACTGGGGGCTTTGGCTCAACCTTTTAATATAAAAAATTCACTGATGTNC | | |
| | | AAAAATGTGAAANAGTGACAATGACAACATATGAAACCCTGNGATTGACAGTCCCCTTGNGNGCACTCCACGGNGCAC | | |
| | | ATGCACCCGCCCACACAGCCTNTGGNGNGGAAACACAAAGCAATGCAAACCAGGGNTACCAANAAGTGCCAACGTGTG | | |
| | | NGTCCTCCGAAAATCCGCAATCACAGATCAGGATCTACTCCAACATTCAGTAACAACAGCACAGCAGCTTCCTAGGAA | | |
| | | CAGGGAGTCCGACTTNTTACTCACGATGGACCAGCNCAAATAAACCGAGCAGAAACAGTACTGCATACTTAAATTTAG | | |
| | | GATAGTCATTCATGAGGATCGTCNCAATTCCAATGTAAGGAACAAACCCTCTTGCTNTCCCCACAACATCTTTCTTCTC | | |
| | | CAGCCAGTGTTGTCCTTGTTTATAGAGACCTCGGTCATCAACAGCATTATTATCTCCTTTGGTTAAAAACTTGATATG | | |
| | | CCCATCTTGCTTTTCATGGGATCTTTANGGACTCGGNGCACTATAGGAANCTCTCTTCCTTNTATCTGAAAACAACGA | | |
| | | TNTTCCCCACCCCGTATAGGGATCTTTCAC | | |
| | Serial clone | UI-27 | | |
| | number: | | | |
| | ACCESS No.: | AB025405 | | |
| | Definition: | Mus musculus mRNA for sid2895p (Mouse microsomal signal peptidase) | | |
| | LOCUS: | | | |
| | Effect: | + (moderately fast) | | |
| Clone # | T7 sequence | CACCCNCTTGGTACCGAGCTCGGATCCCTAGTAACGGCGGCCAGTGTGCTGGAAAGNTCCTTGCTTCTTTTTTGGGG | | |
| 78 | | TNGCGGCCTTGCTCCTGTCCTGCNAGAGAANCNTANAGTGNTTTNACGCNNAAGTGNNCGCGTNGGTTCNGANGTNNT | | |
| | | CNTAGGNCCATGCTCCAGACTCTNNNGTGACNGAAGCNCNANCCNTGGGCNNTACNNGTNNCNCCNNNNGGNTNATCT | | |
| | | TNCAGGCNNNCNATAANGAGAACATNACCGTGGANAANGNNATCCGNCTTTNAGAAAATGTNATCNACCGGATGAAGG | | |
| | | AGGTCCTCTCCATCTGGCTCTAAGGNTNNGCNATATTCNAGCGTNTATGNTGCTTNAGNTTCTGATGAAGATTNTAAA | | |
| | | AGAAGAGTAGCTGNCGAGCTGGCNTTGGAGCAAGCNAAAAAGGAGNCACTGCACCAGANACNCCTTANGCAAGCAAG | | |
| | | GGGACCTATAAAAGAGAAAGGGCTGCATCCAATGAGCAGCTGACTAGAGCTGTCCTTTNGGAAAGAATATCTAGCNA | | |
| | | ANAGGAGCNCATGAANGCNAANCNTCTGGCTCNNCNNCTGGAAGAGANAGATNGAGNGATGCCGGAANCAGNNTGNT | | |
| | | GGAGANNNGAGGCTNAAGTTACCCTGANAACATGCAAGCTCCTTCAACGNTNATTCCACAGGTGGAACATTTTTCTTT | | |
| | | TCCTAGNAC | | |
| | BGH sequence: | TTTTTTTTTTTTTTTTTTNTTNTATTNTTTTTCTAGNCTAAATGGTACCTTTATTAGGTGCCAGGNANGGAANTNNCTA | | |
| | | TAATNANNTANNTGNATNANCAANCCNNNGTNGNCTTTNNATTGNCAAAAAAGCCNNANANTACNNGTAATANAAACCN | | |
| | | TTANTCNANNANANNANGCGCCTNTTTNCTNCNNTGGGGCTTNCTTGAAANAACATTATGGNNNGCCCCGAATNATNTN | | |
| | | NTGTCNAANGAAAATANCATANCCNGGNTTNNCAGGNNNNNTAGGAANACACTGNNGGCCNAACCCATAGCTNTNTTG | | |
| | | NTGGNTGTNCATCNNAAANACGNTTAANGATNCTNCTNCCAGCTTTGTCAAAGNGGAATGTCCCTNAANGCTGGAAGG | | |
| | | AGCCTNAAGGGANNGGANNGGNNAANATGCCAGGANGCTNNAAANNNNGTANATGAAAGGNGNNGGGGNCNGNCTTN | | |
| | | CNNTNTNATNCANTTGGGGGANTGTTAACAGGTTAACAGTGNCGTTTGACTCTGNCANNNGNTTTAAAGNANGGGTTT | | |
| | | TAANCNGNTGNTNATGGCACGTTCTGAATTANTGGNGTCCATNTTCCAGNGCCTGTNTG | | |
| | Serial clone | UI-28 | | |
| | number: | | | |
| | ACCESS No.: | AK000427 | | |
| | Definition: | only partially homologous to: Homo sapiens eDNA FLJ20420 fis. clone KAT02462. | | |
| | LOCUS: | | | |
| | Effect: | −/++ | | |
| Clone # | T7 sequence | AAAGTTCTTCTCTCACGTGGGTTGGCTGCTTGTGCGCAAACACCCGGCTGTCAAAGAGAAGGGCGGAAAACTGGACA | | |
| 79 | | TGTCTGACCTGAAAGCCGAGAAGCTGGTGATGTTCCAGAGGAGGTACTACAAGCCCGGCCTCCTGCTGATGTGCTTC | | |
| | | ATCCTGCCACGCTGGTGCCCTGGGACTGCTGGGGCGAGACTTTTGTAAACAGCCTGTTCGTTAGCACCTTCTTGCGA | | |
| | | TACACTCTGGNGCTCAAACGCCACCTGGCTGGTGAACAGTGCCGCGCATCTCTATGGATATCGCCCCTACGACAAGAA | | |
| | | CATTCANTCCCGGGAGAAATATCCTGGGTTCCCTGGGTGCCGNGGGCGANNGGCTTNCACAACTACCACCACACCTT | | |
| | | CCTTCGACTACTCTGCCANNGAGTACCGCTGGCACATCAACTTNACCACGTTCTTCATCGACTGCATGGCTGCCTGGC | | |
| | | CTGCTTACGACCGNAAGAAAGTTTCTAAAGCTACTGTNTTACCNNGATTANGAGAACTGNAGACNGGAGTCACANGAG | | |
| | | TAGCTGAGCTTTGGGCTTTTGAGATCCTGTTTTAACGGTTTCTGNCAGAGANTTAANATTCTGTGANTAACTAACACT | | |
| | | GGATATTGNTNAATANGGGGGTAANGATGCTTTAACCCCAATCNNGGNCCGNATTCTTTATAAAANGAGAAANNCTTT | | |
| | | TTNATACCCCNTTGAGGGGGNAAAANAATTTNNTTTNNCCTNGGGNTAANCCNTG | | |
| | BGH sequence: | TTTTTTTTTTTTTTTTTTTTTTTTTTTTCCTNTNTGNANAAAAGGTTCAAATTNATNGTNATNATTAAAAGCNGTNGGG | | |
| | | CAAAATNGNANANGCATTNANNANGGGGGCTNAAAAANANANAAGGNNTNGGGGCANGGTAAATCCTAGNTAAANAAA | | |
| | | ANATTNATTACCTCAAAANGGGATCAAAGNTTTNTCATTTNANAANAANACNGNNCCGGAANAGGTAAAAGGCATNATT | | |
| | | AACNCCCCGANAGCAANATCCAGNNGTTAGTNAATCANCANAANATTAAATNTTNGCCAAAAAACGTTNGAAANAGGAA | | |
| | | NTNAAAANCCCAANGNTAAGNTANTNTNGGGANTCCCGTCTCCAGTTNTNTAAANCCNGGNTAAAANAGNANCCTTAAA | | |
| | | AANTTTTTTCNGGNCGAAAGCCAGGCCCAGGGCAGCCATGCAGTCGATGAAAAACGNGGGGAAGTNGANGNGCCANN | | |
| | | GGNACTNANTGGCAGAGNAGTCNAAGGGGAAGGGGNGGGGGAAGTTGGGGAAGCCNTCGCCCACGGNACCCAGGGA | | |
| | | AACCAGGATATTNTCCCGGNATTGAANGTTCTTGTCGAAGGGGCGANATCCATANAGATGCNCGGCACTGTTCNCCAC | | |
| | | CCAGGGGGCGTTGAGCNCCAGAGGGTATCGCAAGAAGGGGCTANCGAACAGGCTGTTTACAAAAGTTTCGCCCCANN | | |
| | | AGTACCAGGGCCCANCGTGGGCAGGATGAAGCACATTANCAGGAGGCCGGCTTGGAAAACCTCCTTTGGAAAATACC | | |
| | | AGTTTTCGGGTTNNAGGC | | |
| | Serial clone | UI-29 | | |
| | number: | | | |
| | ACCESS No.: | NM_009127 | | |
| | Definition: | Mus musculus stearoyl-Coenzyme A desaturase 1 (Scd1) | | |
| | LOCUS: | | | |
| | Effect: | + (slow) | | |
| Clone # | T7 sequence | AAAGCAGCTGATCTCATACAAACACCCCAGACCTCCAGCAGCTCCAGGAGTCAAAAACACCAGCAGTGGACCGAGGTC | | |
| 80 | | CTACAGCTGCCTGCAGCCATGTTGGCCTTCAGTCTGCTCGTGCTGGGTCTGCTCGCGGAAGTGGCACCTGCCAGCTG | | |
| | | TCAACAAGGTCTAGGAAACCTTCAGCCCTGGATGCAAGGCCTCATTGCTGTCGCTGTGNTCTTGGTCCTTGTTGCAAT | | |
| | | CGTCTTCGCCGTCAACCACTTCTGGTGCCAGGAGGAGCCGGAGCCTGGGAGCACAGTGATGATCATTGGAAACAAGG | | |
| | | CAGATGGGGTCCTGGTGGGAATGGATGGCAGATACTCCTCAATGGCATCTGGTTTTAGGTCCAGCGAGCACAAGAAT | | |
| | | GCCTACGAGAATGTTCTGGAGGAAGAGGGCAGGGTCCGCAGCACACCCATGTGACAAGCTTCTCTGTGGCCCTAGTC | | |
| | | CCCAGGCTACAGGGAACATAGAGTTGATCCCCACCATCTNACCCAGCCACTGCTCTACAGGAATCTACTGAAACAAGC | | |
| | | TGACTTCCTCACTCTCTAGAATCACAGNCATCTTAGGCCAGGACTNAGACCAAGCCCAGCACACCAATGTNTGTGACC | | |
| | | CCTGGCTNNTCTNGGCTTNTACCCAGTCTGAGGAGCCTTTNNNAAGGNGGTTAACTNCTTCCCTTC | | |
| | BGH sequence: | TTTTTTTTTTTTTTTTTTCCANAAGTATAAAAAGTCTTNATTTCACAGAAATAAGAGCCANTTCCATAGTTGCAGGAGC | | |
| | | AAGGGNAGGGGAGGAGCTGAGCCACCCTTCACAAGGCTCCTCAGAACTGGGTAGAAGACCCAGGAAAGCCCAGGGTC | | |
| | | ACAGCANATGGNGNGCTGGGCTTGGCTGAGTCCTGGCCTGAGATGACTGTGATTCTAGAGAGGTGAGGGAGTCAGC | | |
| | | TTGTTTCAGTAGATTCCTGTAGAGCAGNGGCTGGNTGANANGGTGGGGANNAACNNTANGNNCCC | | |
| | Serial clone | UI-30 | | |
| | number: | | | |
| | ACCESS No.: | U21049 | | |
| | Definition: | Human DD96 mRNA (a gene selectively upregulated in human carcinomas) | | |
| | LOCUS: | | | |
| | Effect: | + (slow) | | |
| Clone # | T7 sequence | AAAGCGCTCCTCGAGGGTCGGTCCCGCCACGTCTCTTCCCGGTCTCTCTGGCTCTGGAGTGCTTTCCCCCCGCGGAG | | |
| 81 | | GGTGGGAGCGCGGGGACAGACGGGCGAGATGAGCACCATGTTTGCAGACACGCTGCTCATCGTCTTTATCTCCGTG | | |
| | | TGCACCGCGCTGCTCGCCGAGGGCATAACCTGGGTCCTGGTTTACAGGACAGACAAATACAAGAGATTGAAGGCAGA | | |
| | | AGTGGAAAAACAAAGTAAAAAATTAGAGAAGAAGAAGGAAACTATAACAGAGTCAGCTGGTCGACAACAGAAGGAAGAA | | |
| | | GATAGAGAGACAAGAAGAGAAATTAAAGAACAACAATAGAGACCTGTCAATGGTACGGATGAAATCCATGTTCCGCAT | | |
| | | TGGCTTTTGTTTTACTGCCTTAATGGGAATGTTTAATTCCATATTTGATGGTAGAGTGGTGGCAAAGCTCCCCTTCAC | | |
| | | TCCTCTGTCTTACATCCAAGGACTATCTCATCGGAACCTGCTGGGAGATGACACCACGGACTGCTCCTTCATCTTCCT | | |
| | | GTATATCCTCTGTACCATGTCAATTCGACAAAACATCCAGAAGATTCTTGGCCTCGCCCCTTCACGAGCTGCCACCAA | | |
| | | GCAGGCTGGTGGATTTCTTGGCCCACCACCTTCGTCTGGGAAGTTTTNCTTGAAGGAAAGCAGAATTCTGAATTTCC | | |
| | | TGNCATACTTTTTAGACATTCACATCAGACTTACCGAGCACCTGGCCACAATCTAGGTNGGGGTAATCTCACTATGGA | | |
| | | TATGAACCAATGAGAACCCTGNTTACTAAAGGGAAAATGCTATGGNCACCGGATGGCTTCNTTNAGTAATAAGTGGCC | | |
| | | CCNNTNTGGGTACCATTTGGAAGGNTTAATGTAACCCCCAACCATCAANCTTTCTTGCTTNNCTTG | | |
| | BGH sequence: | TTTTTTTTTTTTTTTTTTTTTTGGTAAAAGAACATGACAAAACTTTATTTTAGCTTTTTGGGCCAATGCTTATCCTTAT | | |
| | | GTTCAGATTACAGAAAATACTCTTTTTAATATGCAAAAAAATCAAACATTTCTCTAACTGAAACATCTAAAAATTTGGC | | |
| | | TCATTGTCGAACACAAAGTGACATATTTGGCTTTTTAAAAAACACAACAACTAATTACAGTGAAGTTGTTACATAAACT | | |
| | | ATTTTTCTCCTAATGTTCATGGACAGTCTGCTTCTGTGTATATGGCACATACACTGAAATACTCAAGTGAGACAAGAA | | |
| | | AGATGATGTTTGNGGTTACATAAGCCATCCAAATGTAACCAGTGAGTGCACTTATTACTAAAGAAGCCATCCGTGACC | | |
| | | ATAGCACTATCCCATTAGTTAAGCAGTGTTCTCATTTGATCATATCCATAGCTGAGAAATAACACAAACCTAGAATGTG | | |
| | | GCCAGGTGCTCTGTAAGTCTGATGTGAATGTCTAAAAAGTATGACAGGAAATTCAGAATTCTGCTTTCCTTCAGGAAA | | |
| | | ACTTCCCAGACGGAGGTGGTGGGCCAAGAAATCCACCAGCCTGCTTGGTGGCAGCTCGTGAAGGGGCGAGGCCAAG | | |
| | | AATCTTCTGGATGTTNTGTCGAATTGACATGGTACAGAGGATATACAGGAAGATGAAGGAGCAGTCCGTGGNGCATC | | |
| | | TCCCCAGCANGGTTCCCGATGAGAAAGTCCNTGGATGTAAAGAACAGANGANTGAAAGGGGGAGCNTTTGCCCCCAC | | |
| | | TTTTACCCTTCAAATAATGGGAATTAAACCANTTCCCAATTANGGCGGTAAAAACCAAAAGGCCCAATTGGGGAAACA | | |
| | | TGGGAATTTCATCCCGNANCCATTTGGNCAGGGCCTNCTAAATGGGGGGNCCTTTNAAANTCCCCTTTCTTGGGCCCT | | |
| | | TNTAAAACTTNCNTTCTNTTGGGGGGGCCNACCCAA | | |
| | Serial clone | UI-31 | | |
| | number: | | | |
| | ACCESS No.: | AF070626′ and AB020980 (are in principle identical) | | |
| | Definition: | Homo sapiens clone 24483 unknown mRNA (AF070626), or | | |
| | | Homo sapiens mRNA for putative membrane protein (AB020980) | | |
| | LOCUS: | | | |
| | Effect: | + (slow) | | |
| Clone # | T7 sequence | TTTCGCCCCCCNTTTGGTACCGAGCTNGGATCCTNTAGTAACGGCCGCCAGTGTGCTGGAAAGGCGACATGGGGAGG | | |
| 82 | | CGCGGAGGCGACACCGGAAGTGGCTGTGGTCCGGGTCGGCCCGAGGGGGACTCAGCGCCCGCAGCAACCACCCGCG | | |
| | | CGGCGGCGGTCAGAGCTNANACCCGTGCGGGTGGGCGTGGCGGGCGCCGGGACACAGCTCCGACTGTTCGCTGTCG | | |
| | | GCGCGGGGCGGCGCCGCGTCGCTCGCCTTNACCAGCTGCCATGAGCGAGCGCCTCCGCCCCAGAAAAAGGAGAAGAA | | |
| | | ATGGCAGTGATGATGACAACCACCCTCCTCCCCAGACCAAAAGGAGCAGTAGGAACCCCATCTTCCAGGACTCCTGGG | | |
| | | ACACAGAGTCTTCAAGCAGCGACAGTGGTGGGAGCAGCAGCAGCAGCAGCATNAACAGCCCAGACAGGGCCAGNGGG | | |
| | | CCAGAGAGCAGCCTGAGCCACACCATCCCCGGATCCTGCCCCAGCACCCCCCAGCCGATGCCTGAGCAGTCTGCACTA | | |
| | | TGCCAAGGCCCTTACTTCCACATNAACCAGACCCTGAAGGAGGCTCACTTTCATAGCCTACAGCACCGAGGCCGGCCG | | |
| | | CCGACATGATGCTCCTCCGGCAGNTTCTTGCCTTCTGTGAAGGGACAGCACTGTGCAGATTGNATATT | | |
| | BGH sequence: | CGAACGCNAGTTCTAGCATTTAGGTGACACTATAGAATAGGGCCCTCTAGATGCATGCTCGAGCGGCCGCCCTTTTTT | | |
| | | TTTTTTTTTTTCTGTGTGCAACTTTTAAAAACAAATCATTAAGTTGAAATATCCAATCTGCACAGNGCTGTCCCTTCAC | | |
| | | AGAAGGCAAGAAACTGCCGGAGGAGCATCATGTCGGCGGCCGGCCTCGGNGCTGTAGGCTATGAAAGTGAGCCTCC | | |
| | | TTCAGGGTCTGGTTGATGTGGAAGTAAGGGCCTTGGCATAGNGCAGACTGCTCAGGCATCGGCTGGGGGGNGCTGG | | |
| | | GGCAGGATCCGGGGATGGNGNGGCTCAGGCTGCTCTCTGGCCCACTGGCCCTGTCTGGGCTGNNGATGCTGCTGCT | | |
| | | GCTGCTGCTCCCACCACTGTCGCTGCTTGAAGACTCTGTGTCCCAGGAGTCCTGGAAGATGGGGTTCCTACTGCTCC | | |
| | | TTTTGGTCTGGGGAGAGGGNGGGNTGNCATCATCACTGCCATTNCTTCTCCTTTNTCTGGGGCGGAGGCGCTCGCTC | | |
| | | ATGGNAGNTGNNGAAGGCNANCGANNCGNNNCCGCCCNNNCCNNCAGNNAACAGNCNNNGCTGNGNCCGNN | | |
| | Serial clone | UI-33 | | |
| | number: | | | |
| | ACCESS No.: | U92989 | | |
| | Definition: | Homo sapiens clone DT1P1E11 mRNA, CAG repeat region (partial homology) | | |
| | LOCUS: | HSU92989 | | |
| | Effect: | + (slow), laddering | | |
| Clone # | T7 sequence | ANCGACCCCCCCNTTNGACCGAGCTCGGATCCTCTAGTAACGGCCGCCAGTGTGCTGGAAAGGCTGATTGCTGAGGT | | |
| 83 | | GGGAGTGGGCCCANCGCCCGGNGGCCGCAGCTCACGCGCAACCTGCGCCATGGCCGCCTCCGCCGCCTCCTCCGAGC | | |
| | | ATTTCGAGAAGCTGCACGAGATCTTCCGCGGCCTCCTTGAAGACTTACAAGGGGTGCCGGAGCGGCTGCTGGGGACC | | |
| | | GCGGGGACAGAAGAGAAGAAGAAGCTGGTCAGAGATTTTGATGAAAAGCAACAGGAAGCAAATGAAACGTTGGCAGA | | |
| | | GATGGAGGAAGAACTACGATATGCACCCCTGACTTTCCGTAACGCCATGATGTCTAAGCTGCGAAACTACGGGAAGGA | | |
| | | CCTTGCTAAACTCCACCGTGAGGTGAGAAGTACACCTCTGACAGCCGCACCTGGAGGCCGAGGAGACCTGAAGTATG | | |
| | | GCACGTATGCCTTGGAGAACGAGCATTTGAATCGACTACAGTCTCAAAGAGCATTACTCCTACAAGGCACTGAAAGCC | | |
| | | TGAACCGGGCTACCCAAAGCATTGAGCGTTCTCATCGGATTGCCACAGAAACTGATCAAATTGGTACAGAAATCATAG | | |
| | | AAGAGTTGTNGGAGCAACGAGACCAGTTGNAACGTACTAAGAGCAGACTGGTAAATACAAATGAA | | |
| | BGH sequence: | TTTTTTTTTTTTTTTTTTTTTAAGTTTGAAGGAGTCATAAACAACATTTATTACCTTAGTATATCATACTGGTCTTGTT | | |
| | | GCTGTTCCTTCACATTTTCGAGGTTTTCCATTGCCCTCCCTCCCATAGTCTGACAAGACAATACATTCTTGAACTGTTA | | |
| | | GCCCACAGCAGCAACACATATCCCTATGTAAAACCATTCATTCAGAGTAAGATGCTGGTCCACAAGGCTTTCCCTACA | | |
| | | GAAGTTCAATGGTGTCGAAAGAATTTGTAATACACCAGACCGACCAGGATGGCTAGCTCCAGCAAGATGATGACGGA | | |
| | | GAGCAGCAACTTGTTGGTTATCACTTTTCTGGACATTGAGCGAAGAATCTTCCGGCTTTTGCTCAAATTTTCATTTGT | | |
| | | ATTTACCAGTCTGCTCTTAGTACGTTCCAACTGGTCTCGTTGCTCCCCCAACTCTTCTATGATTTCTGTACCAATTTGA | | |
| | | TCAGTTTCTGTGGCAATCCGATGAGAACGCTCAATGCTTTGGGTAGCCCGGTTCAGGCTTTCAGTGCCTTGTAGGAG | | |
| | | TAATGCTCTTTGAGACTGTAGTCGATTCAAATGCTCGT | | |
| | Serial clone | UI-54 | | |
| | number: | | | |
| | ACCESS No.: | AF035208 | | |
| | Definition: | Mus musculus putative v-SNARE Vti1b mRNA (soluble NSF attachment protein receptor) | | |
| | LOCUS: | | | |
| | Effect: | + (slow), no laddering (?) | | |
| Clone # | T7 sequence | TNTGACGCCGTTCTAGCATTNAGGTGACACTATAGAATAGGGCCCTCTAGATGGATGCTCGAGCGGCCGCCCTTTTTT | | |
| 84 | | TTTTTTTTTTTTTTCCGTTATAATAGCCATCTTTATTTGTAAAAATCCAGATATAAAACGTAATCTTTCAGTCTTTCCA | | |
| | | GGTTTTCCTTTTTTACAAAAACAAAAAGGCACGTATAAACCTTGCCCGCTGTCGTCCCCGTACACGGNGTTTCTCAGG | | |
| | | CAGCCCTCCCCCCCGCCCCGCCCCCCGTTACAGCTACATGCTTCATTCCAGGACGTCTGCATCCCCACATGCTTTGGN | | |
| | | GCTTTCCTACCAGGGTAGAGTTCCGAGCTCCAAGACTTGAAGTACACAAAGAGGGGGTAGGGGTGGGTGCAGNGTG | | |
| | | TGGCACAATGTTCCACGGCGTGCAGGGCAGNGGGCTAGTAGTAGGTCTCCTTCTCCACCCAGCCGCCAGGGCGCCGC | | |
| | | CTGATAATGAGCTTCCGCACCTCGTCATACACAAAGATGAGAAGGGAGTAGGGGAAGGCACAGAACCACCATGTAGG | | |
| | | TTTGAGGGGATACATCCTAAAGGCTGCCCCCATCCCGGGGCAGTAGGATAAGAAAGCAGCAAGGGCTGTCTCTTCAA | | |
| | | AGAGGCCAAATATCAAGATCTTGGTCTTCATTCCCTGCTGGA | | |
| | BGH sequence: | TTCCANCCCCCCTTGGACCGAGCTCGGATCCTCTAGTAACGTCCGCCAGTGTGCTGGAAAGGGGTTGCCATGGGGAT | | |
| | | TGTTGGCTCTGATGTGTCCAAGCAAGCTGCTGACATGATCCTTCTGGATGACAACTTTGCCTCCATTGTGACTGGAG | | |
| | | TAGAGGAAGGTCGTCTGATTTTTGATAACTTGAAGAAATCCATCGCTTACACCCTAACCAGTAACATTCCGGAAATCA | | |
| | | CCCCCTTCTTGATATTTATTATTGCAAACATTCCACTGCCCCTGGGGACTGTGACCATCCTCTGCATTGACTTGGGCA | | |
| | | CTGACATGGTTCCTGCCATCTCCCTGGCCTACGAGCAAGCTGAGAGCGACATCATGAAGAGGCAGCCCAGAAACCCCA | | |
| | | AAACGGACAAACTTGTGAACGAGCGTCTGATCAGCATGGCCTATGGACAGATCGGTATGATCCAGGCCCTGGGAGGC | | |
| | | TTCTTCACTTACTTTGNGATTCTGGCTGAGAACGGTTTCCTGCCCTTTCACCTGTTGGGCATCCGAGAGACCTGGGAT | | |
| | | GACCGCTGGGTCAACGATGTGGAGGACAGCTACGGGCAGCAGTGGACCTACGAGCAGAGGAAGATCGTGGAGTTCA | | |
| | | CCTGCCATACAGCGTTCTTTGTCAGTATTGTGGTAGNGCAGTGGGCCGACTTGGTCATCTGC | | |
| | Serial clone | UI-60 = UI-56 | | |
| | number: | | | |
| | ACCESS No.: | NM_012504 | | |
| | Definition: | Rattus norvegicus ATPase. Na + K + transporting, alpha 1 polypeptide (Atp1a1), mRNA. | | |
| | LOCUS: | | | |
| | Effect: | + (slow) | | |
| Clone # | T7 sequence | TTCGNCCCNCTTGGTACCGAGCTCGGATCCTTAGTAACGGCCGCCAGTGTGCTGGAAAGGTCTGCGGCCCTCGCAGA | | |
| 85 | | ACTTCCAGCAGCGACATGTTGGGCCAGAGTATCCGGAGGTTCACGACCTCCGTGGTCCGTCGCAGCCACTATGAGGA | | |
| | | GGGTCCGGGGAAGAATTTGCCATTTTCAGTGGAAAACAAGTGGCGGTTGCTGGCTATGATGACCGTGTACTTTGGAT | | |
| | | CTGGGTTTGCCGCACCTTTCTTTATAGTAAGAGACCAGCTACTTAAAAAATAAGGATATTTAATTCATCCCTTTAACAG | | |
| | | AATGAAGAAAGTTTAAGAGGTGATCTGAAAATTGGATTAAACTCTTGAACTCTTATACTAGAAAAAATTGTAAATAAA | | |
| | | CTAATGACATAAAGATTCAAAAAAAAAAAAAAAAAAAAAGGGCGGCCGCTCGAGCATGCATCTAGAGGGCCCTATTCT | | |
| | | ATAGTGTCACCTAAATGCTAGAGCTCGCTGATCAGCCTCGACTGTGCCTTCTAGTTGCCAGCCATCTGTTGTTTGCCC | | |
| | | CTCCCCCGTGCCTTCCTTGACCCTGGAAGGTGCCACTCCCACTGTCCTTTCCTAATAAAATGAGGAAATTGCATCGCA | | |
| | | TTGTCTGAGTAGGTGTCATTCTATTCTGGGGGGAGAGNAGANGCAGGACAGCAAGGGNGAC | | |
| | BGH sequence: | TTTTTTTTTTTTTTTTTTTTGAATCTTTATGTCATTAGTTTATTTACAATTTTTTCTAGTATAAGAGTTCAAGAGTTTA | | |
| | | ATCCAATTTTCAGATCACCTCTTAAACTTTCTTCATTCTGTTAAAGGGATGAATTAAATATCCTTATTTTTTAAGTAGC | | |
| | | TGGTGTCTTACTATAAAGAAAGGTGCGGCAAACCCAGATCCAAAGTACACGGTCATCATAGCCAGCAACCGCCACTTG | | |
| | | TTTTCCACTGAAAATGGCAAATTCTTCCCCGGACCCTCCTCATAGNGGCTGCGACGGACCACGGAGGTCGTGAACCTC | | |
| | | CGGATACTCTGGCCCAACATGTCGCTGCTGGAAGTTCTGCGAGGGCCGCAGACCTTTCCAGCACACTGGCGGCCGTT | | |
| | | ACTAGTGGATCCGAGCTCGGTACCAAGCTTGGGTCTCCCTATAGNGAGTCGTATTAATTTCGATAAGCCAGTAAGCA | | |
| | | GNGGGTTCTCTAGTTAGCCAGAGAGCTCTGCTTATATAGACCTCCCACCGTACACGCCTACCGCCCATTTGCGTCAAT | | |
| | | GGGGCGGAGTTGTTACGACATTTTGGAAAGTCCCGTTGATT | | |
| | Serial clone | UI-61 | | |
| | number: | | | |
| | ACCESS No.: | NM_007749 | | |
| | Definition: | Mus musculus cytochrome c oxidase subunit VIIc (Cox7c), mRNA | | |
| | LOCUS: | | | |
| | Effect: | −/++ (moderately fast) | | |
| Clone # | T7 sequence | TTCCGCCCCCCCNTGGTACCGAGCTCGGATCCANTAGTAACGGCCGCCAGTGTGCTGGAAAGATGAATTCAAAGAGT | | |
| 86 | | GCCCAGGGTCTGGCTGGTCTTCGAAACCTTGGGAACACGTGCTTCATGAACTCAATTCTTCAGTGCCTGAGCAACACC | | |
| | | CGAGAGCTGAGAGATTACTGCCTCCAGAGGCTGTACATGCGGGACCTCGGCCACACCAGCAGCGCTCACACGGCCCT | | |
| | | CATGGAAGAGTTTGCAAAACTAATCCAGACCATATGGACGTCGTCCCCCAATGATGTGGTGAGCCCATCTGAGTTCAA | | |
| | | GACCCAGATCCAGAGATATGCGCCACGCTTCATGGGCTATAATCAGCAGGATGCTCAGGAATTCCTTCGTTTCCTTCT | | |
| | | GGATGGTCTCCACAATGAGGTGAACCGGGTGGCAGCAAGGCCTAAGGCCAGCCCTGAGACCCTTGATCATCTCCCTG | | |
| | | ATGAAGAAAAGGGGCGACAGATGTGGAGGAAGTATCTGGAAAGGGAAGACAGTCGGATTGGGGATCTCTTCGTTGG | | |
| | | GCAGCTGAAGAGCTCCCTCACATGCACCGATTGTGGCTACTGCTCTACAGTCTTCGATCCCTTCTGGGATCTCTCGTT | | |
| | | GCCCATCGCAAAGAGAGGTTACCCTGAGGTGACGTTAATGGATTGTATGAGGCTCTTCACCAAAC | | |
| | BGH sequence: | TTTTTTTTTTTTTTCTGAATTTTTTAAAAAAAACTTAGGACGTGGGGCCACCACAGGGAAGAGGGAAGGGCCGCAGC | | |
| | | TCCTCAATGCTACATACGGGAGGGTGGACTGGCCAGTTCATAGAAGAGCAAATAGGCGTCGCTGGTGCGCACTTGGC | | |
| | | TGGAGGACATGGGTGTGACACTGGAATCATTGAAAGTGTGCCATTCGCCTGTAACCGGACTTCGGCAGTAGGCTGTA | | |
| | | TAGNGGCCTCCCATGGNGGTTCCGGAGTGATTGGACACAGCATACAGGTTGTAAACAGCATGGTTGGNGTTTTCTGA | | |
| | | AGCAAATTCTCTCAAGTCCAGGTCTCTTAGNGGGAAATTCACAAATGTTGNGAGCTTGCTGGTTCGTATCCTGGATTC | | |
| | | TGAGAATCGCTTCAGGTGGAGCACCAAGATCTTTGGGAACCTCTGGACAGAGAACTTTTTTATGCATCGCTTTCTGG | | |
| | | CTCGGCAGCGGCAGCAAGTTGGCTTCTCATCACCATCCAATATGTCCTCTTTGGTGAAGAGCCTCATACAATCCATTA | | |
| | | ACGTCACCTCAGGGTAACCTCTCTTTGCGATGGGCAACGAGAN | | |
| | Serial clone | UI-64 | | |
| | number: | | | |
| | ACCESS No.: | AF079565 | | |
| | Definition: | Mus musculus ubiquitin-specific protease UDP41 (Ubp41) mRNA | | |
| | LOCUS: | | | |
| | Effect: | + (Slow) in 293T, ++ (fast) in HeLas, laddering (293T) | | |
| | Clone | | | |
| Clone # | T7 sequence | AGGGCGCCTGAGTGAAAAGNGNGGCACCATGGCCTCTGNGCTGTCCTACGAAAGTCTGGTACACGCCGNGGCCGGA | | |
| 87 | | GCCGNGGGAAGNGNGACTGCCATGACAGNGTTCTTCCCCTTGGATACTGCTAGACTTCGGCTTCAGGTCGATGAGA | | |
| | | AAAGAAAGTCAAAAACGACGCATGCAGNGCTCCTGGAGATANTTAAGGAAGAAGGCCTCCTGGCACCATACCGAGG | | |
| | | ATGGTTTCCAGCTATTTCCAGTCTCTGCTGCTCCAATTTTGTCTATTTTTACACTTTTAATAGCCTCAAAGCAGNGNG | | |
| | | GGTCAAAGGTCAGCGTTCTTCTACAGGAAAAGATCTCGNGGNNGNGTTNGTAGCAGGAGNGGTGAATGNGCTGCTG | | |
| | | ACGACTCCGCTCTGGNNGGTAAACACCAGACTGAAGCTGCAGGGGGCAAAATTTCGGAATGAAGACNTTATACCAAC | | |
| | | TAACTANAAAGGCATTATCGNTGCATTCCACCAGATTATTCGNGNTGAAGGNATCTTGNCTCTGNGGAATGGCACCT | | |
| | | TCCCCTCCCTTGCTGATGNACTTCAACCCTGCCATCCAATTCATGNTCTATNNNGGCTTAAAACGGCAGCTTCTACCG | | |
| | | AAACGNCTGANNCTCTCTTTCTCTGCNTNNGTCCNTCATTTGNCGCACTANNCAAAGCTNATTGTCNCCACAGCNNCC | | |
| | | CTATCCCCTNCAGANCGGTNCCTGTCNATTCTGAGGCNTNCAC | | |
| | BGH | TTTTTTTTTTTTTTTCAGGGTTTCAATATTTTCATATCAGTCTAACCTCTACCCCCAAATACAACCAAACACCATAAAA | | |
| | sequence | ACACCAAAAAACCCCATAAAGGTGACAGGCTTAGCTGGCTATACTCCCTGCTTAGCCTTTGATGCCTTTACAGCCAA | | |
| | | TGACTGGAATGTCTGNGGNGNGGATGGGACCAACGGGAAAGGAGAACTGTTCCTCTTCTCCCTGTGCAGCACCTGC | | |
| | | GTCTCCACACTGTCCTGNGGGCGGGCCTCAGNGCTTATGTGTGCTCTTCAGGCCCATTACGGNGAAGGTAGCAGCT | | |
| | | GTCAGTTTCTCGTACACAAGGAACATGAGGGCGGCTGTGAGCACTGTCTGCAGCAGCTTAGCTTCCAGGCCTTTGT | | |
| | | AGAGTCCCATTATTCCAAAGCGCTTGACTCGCTGGTGAAGAAGAGAGAGAACATTCCGAAGACTTCCCAGGGTCCT | | |
| | | GTTTTCTGGGTTCAGTCTATGACGTCCAAACCTCAGAATTGACTGTACCGTCTGCATGGGATAGGTGACTGNGGNG | | |
| | | GCAATCGCTNTGGCTATTGCGCCAATGATGAACACATNCAGAGAAGAGAGCTTCATCCGNTTCTTTAGAAGCTGCCG | | |
| | | TTTTAAGCCTTCATAGAACATGAATTGNATGGCAGGGTTGAAGACCAACAGCAAGGAGGGGAAGGTGCCATTCCAC | | |
| | | AGAGCCAAGATCCCTTCATCTCGAATAATCTGGTGNAATGCATCGATAATGCCTTTGTAGTTAGCTGGNATAATGGC | | |
| | | TTCAATT | | |
| | Total | LOCUS MMU6341 1700 bp mRNA ROD Jan. 8, 1999 | | |
| | cDNA, | DEFINITION Mus musculus mRNA for peroxisomal integral membrane protein PMP34. | | |
| | EST cluster | ACCESSION AJ006341 | | |
| | Motifs | | | |
| Clone # | T7 sequence | AGTAGAGCCCCTACGGTCATGGCGGCTACCGCCCCCAAGGCCGGGGGTTCAGCTCCTGAGGCAGCGGGTTCTGCC | | |
| 88 | | GAAGCTCCGTTGCAGTACAGCCTTCTTCTTCAGTACCTGGAGGGCGACAAGCGTCAGCCCCGGCTTCTGGAGCCTG | | |
| | | GCAGCCTGGGCGGGATCCCGAGTCCTGCCAAGAGCGAGGAGCAGAAGATGATCGAGAGGGCAATGGAAAGNTGCG | | |
| | | CCTTCAAGGCTGAGCTGGCCTGNGTAGGAGGGTTNGNCTNGGGAGGCGCGNTNGNTATCTTTACTGCTGGCATTGA | | |
| | | TACCAACGTAGGCTTTGACCCAAAGGACCCTTACCGGACACCAACTGCAAAAGAAGTCCTGAAAGACATGGGACAGA | | |
| | | GAGGAATGTCCTATGCCAAAAACTTTGCCATTGAGGGCCGCCATGTTTTCATGTACTGAGNGNCTGGTAGAGTCTT | | |
| | | ACCGGGGAAAGTCGGACTGGAAGAACAGCGTCATCAGNGGCTGCATCACTGNCGGAGCCATCGGCTTCCGAGCTGN | | |
| | | AGTAAAGGCCGGGGCCATAGGTTGNGGAGGGNATGCTGCTTTCNCTGCTGCNATCGNTTNTTACCTACGNNGAAGG | | |
| | | AAANAGCCTNCTAAGGAAAGAGGACNCCAGCCNCTTNAGAGCTGCTCTG | | |
| | BGH | TTTTTTTTTTTTTTTTTTTTTTTTTTTTTTTTTTTGCCTCGATGGCATAAGTTTACTTTGCCCTGCCCTGGGACTCAT | | |
| | sequence | GATGGATACTTGCACACAGAAGGAAGAAGAGCTTCCCGGNTATGGGCAAACACTGCCACTTCCCTATTCAAAGCTGG | | |
| | | GGAGCTGAGCCAGGTCAGAACTGAGCCTGGTTCAAAGCTTAANGCGTAACAGTCCACACACTGCCTCCTGATCTGTT | | |
| | | TACTGCAAGGAGACAATNTGCCATACCACCTNCCCTGAAAAACCTGGTTGGAATTCAAGTGGAAAAGTATGGTTGTT | | |
| | | CCAANGGCTGCAGAGAGCGATCGAGCAGCGAGGNGNGTCCTTNACACCATNAACTGTAGAGGGATGCACTTGGCTT | | |
| | | CACCCCTGAAGCAGTCCAAGTGTGTAGAAACTGNTCCACAGAGCAGCTNTGAAGGNGGCTGGCGTCCTCTNTCCTTC | | |
| | | CCGGCTGNTTCCTTCACCGTAGGTAATAATCGATTGCAGCAGAGAAAGCAGNAAACCNTNCACAACCTATGGCCCCG | | |
| | | GCCTTTACTCCAGCTNGGAAGCCGATGGCTCCGCCAGTGATGNAGCCACTGATGACGCTGNTCTTCCAGNCCCGACT | | |
| | | TTCCCCGGTAAGACTNTACCAGACNCTTAGTACATGAAAACATGGCGCCCACAATGGCAAAGTTTTTGGCATAGGAC | | |
| | | ATTCCTCTCTGNCCCATGNCTTTCNGGACTTCTTTTGCAGATGNTGCCCGGAAAGGGCNCTT | | |
| | Total | see | | |
| | cDNA, | LOCUS AF223950 1062 bp mRNA ROD Jan. 27, 2000 | | |
| | EST cluster | DEFINITION Mus musculus TIM22 preprotein translocase (Tim22) mRNA, complete cds; nuclear gene for mitochondrial product. | | |
| | | ACCESSION AF223950 | | |
| | Motifs | | | |
| Clone # | T7 sequence | AAGGCCGGGAGGATGCGGCCGGAGCCCGGAGGNTGCTGCTGCCGCCGCCCGATGCGGNCGAACGGCTGCGTNAAG | | |
| 89 | | AACGGNGAAGNGAGGAACGGNTACTTGAGGAGCAGCACCGCCACCGNCGCGGCTGCCGGCCAGATTCATCATGTAA | | |
| | | CANAAAATGGAGGACTGTACAAAAGACCGTTTAATGAAGCTTTTGAAGAAACACCCATGCTGNCNGCTGNGCTCACA | | |
| | | TATGNNGGCTATGGCGTACTCACCCTCTTTGGATATCTTCGAGATTTCTTGAGGCATTGNAGAATTGAAAAGNGCCA | | |
| | | CCATGCAACAGAAAGANAANAACAAAAGGACTTTGCGNCCTTGTATCANGNTTTTGAAANCTTCTATNCAAGGAACCT | | |
| | | CTACATGAGAATCANAGACAACTGGANTCGACCTATCTGTANTGNGCCTGNAGCCAAGGTGGATATCATGGNGAGAA | | |
| | | AATCTNATGACTATAACTGGTCATTCAAGTACANANNNAATATNNTTANAGGTGTCATAAACATGGNTTGCTACACCT | | |
| | | ATCTTGTNTTTGCGAGGNACACTNNATCATNTCANGNNGCCCCTNCTGAANTCCTCACNGAGTATTNCAGCAGCCCT | | |
| | | GNGCAANCNCTCGCANNNNANTNCTAACCTNTACANGCATNCCTNAACT | | |
| | BGH | TTTTTTTTTTTTTTTTTTTTTTAGCAGTTTGGNTTTTTTACTATTTACAAAATGCCATTTGGAGTGAAGGTGGCCAC | | |
| | sequence | CTTCAGTAGCTTCAGAAATGTCTTTCACATGAAGNGGTCACTGAAGNGNGGTTCCTGGAATTGGCTCAGAAAGGCC | | |
| | | ACACACTGTCCTGGGAGGAATTATCCCCCTCTAGGGAGCACCAGAAAGGCTCAGTCTTCTGTCTCTTCATAGGTAGT | | |
| | | CTCATCAAAGGGCCTGTCCAGTAGAGGCACCAGCCGGNGGCGAGAGTACTTTAGCTGCAGCAGATCCCCAACTTCA | | |
| | | TCTATCTCCTTCAAAGCAGNGTCAAGTATTTCTTTGGTATGAGCTGCTGACAGGCAAAATCTGGCTCTGGACTCAAT | | |
| | | GATCGGGGTAGCAGGAAATCCCACCACAACTACACCAATGTTCCGCTTCAGCATCTCTCTTCCAAAGGCGCCAATTT | | |
| | | TGGCCGGCATGTAGAGCATCAAAGGCACCACCGGGGAGTCTTCATTGCCATAGATGATGAACCCCATTTCCTTCAG | | |
| | | GCGTCTCCTGAAATACCTGGNGTTCTCAGCCAACTGCTGTATACATTCTTTGCCAAGACTGGAGCCATCCTGCCCCA | | |
| | | TGATGNACTTCATNGAGGTGATAATCTGTTCCATCACAGGCGGTGACATCGGACCGTGGCATACACAGCACTGTGN | | |
| | | GAATGTNTGCGCAGGTAGNCTATCNGGTGCTTCTTCCTCCCNATGNATCCCTCTTGAANGCACCNNAGCTNTTTTNT | | |
| | | GAATGTTCCCAT | | |
| | Total | LOCUS | NM_011479 1986 bp mRNA ROD Jan. 25, 2000 | |
| | cDNA, | DEFINITION Mus musculus serine palmitoyltransferase, long chain base subunit 2 | | |
| | EST cluster | (Sptlc2), mRNA. | | |
| | | ACCESSION NM 011479 | | |
| | Motifs | | | |
| Clone # | T7 sequence | AGCGGATCTTCGGGCCGGGAGGACATTCGGCCTCTGTGAGCCGCAACCTTGCCCAGCGAGCGGTTGGGNGTTCGC | | |
| 90 | | CATCTTAGGAGGATGTTGTCGTCCGTAGCGCACCTGGCGCGGGCGAACCCCTTCAACGCGCCACACCTGCAGCTGG | | |
| | | TGCACGATGGCCTGTCGGGTCCCCGCAGTCCCCCAGCTCCGCCCCGGCG1TCCCGCCACCTGGCCGCCGCCGCCGN | | |
| | | GGAAGAGTACAGTTGNGAATTTGGCTCCATGAAGTATTATGCACTGAGNGGCTTTGGNGGGGTCTTAAGTTGAGG | | |
| | | GCTGACACACACTGCTGNNGTTCCCCTGGACTTAGTAAAGNGCCGCATGCAGGNGGACCCTCAGAAGTACAAAGGC | | |
| | | ATATTTAATGGATTCTCCATTACACTGAAAGAAGATGGCGTTCGNGGTTNGGCTAAAGGATGGGCCCCAACTTTGAT | | |
| | | NGGCTATTCCATGCAAGGGCTCTGCAAATTCGGCTTTTATGAAGTCTTCAAAGCCTTATATAGCAACATACTTGGTG | | |
| | | AGGAAAACACCTACCTGTGGCGCACATCACTGTATTTAGCTTCTTCTGCCAGAGCTGAATTCTTCGCTGACATTGCC | | |
| | | CTGGCTCCTATGGAAGCTGCTAAAGTTCGAATTNAAACCCAGCCTGGGTATGCCAACACCTTGAGGNAAGCTGTTCN | | |
| | | CAAAATGTATAAAGAGGAANGCTTAAATGCGTCTACAAGGGCGATGCTCCTCTGTGGATGAGACAGATCCCCTACAC | | |
| | | CATGATGAAGTTCGCT | | |
| | BGH | TTTTTTTTTTTTTTTCAGGAATAGATGGGTTTATTTAGCATTAAGTTTGAAACTCTTGCTTTAAGCAAGGGTACTAC | | |
| | sequence | AGTAATTATATCAGGAACAGACATTTCCTACACTGTCAAATATATAAAAGTTCCTTTGCACTTTCAACACTGATCAAC | | |
| | | AAGCAGATTCAGTCCATGTTACTTTGATGTATCTACTCAGTTAACCCAAGCTTCTTCTTCAGAGACTCTGGCATCTCA | | |
| | | GGGGGAGGAGGGCGAGGGAGCCTGAAGTAGACCTTCACAGAGTCGTAGATGAACCACTGTAGNGCAGTCAGAGTG | | |
| | | CCAATCATGATGATTCGGGCGAAGAGcCCCTTCCACACACCTCTGAAGCCCAGCCTCTGCAGGACCTGAGACGCGGA | | |
| | | GCTGCCTTTCTCTTTATTCAGCACAGAGACCACAGAGTCAGCAGGGNGGGAGACGATGGCACAGAAGACTCCAGCT | | |
| | | ATGTAACCTGCCACAAATGTCACAACCAGCTGCTCTGCCTTTGTACATTCACTTCGGGGCTTGGGAACCACAAATTT | | |
| | | GTACAAAGCTTCAACAGTACGTTCAAAGCAGGCGAACTTCATCATGGNGTACGGGATCTGTCTCATCCACAGAGGAG | | |
| | | CAACGCCCTTGTAGAACGCATTTAAGCCTTCCTCTTTATCATTTTGGGAACAGCTTCCCTCAAGGTGTTGGCATAAC | | |
| | | CAGGCTGGGTTTTGAATTCNNAACTTTAGCAGCTTNCATAGGAGCCAGGGCAATGTCAGCGN | | |
| | Total | Rat sequence: | | |
| | cDNA, | | | |
| | EST cluster | LOCUS RATMPT 1263 bp mRNA ROD Apr. 27, 1993 | | |
| | | DEFINITION Rat mitochondrial proton/phosphate symporter mRNA, complete cds. | | |
| | | ACCESSION M23984 | | |
| | | Human sequence: | | |
| | | LOCUS SSMPCP 1330 bp mRNA PRI Jun. 1, 1992 | | |
| | | DEFINITION H. sapiens mRNA for mitochondrial phosphate carrier protein. | | |
| | | ACCESSION X60036 | | |
| | Motifs | | | |
| Clone # | T7 sequence | AGGCACTGCTGGCGACATGGCCGACACGGACCCGGGCTATCCCCGCTCGTCCATCGAGGATGACTTCAACTACGGC | | |
| 91 | | AGCTGCGNGGCGTCGGCCAGCGNGCACATCCGCATGGCCTTTCTCAGAAAAGTCTACAGTATCCTCTCTCTGCAAGT | | |
| | | CCTCCTGACTACAGNGACCTCTGCCCTGTTCCTGTATTTCCAAGCTCTGCGGACATTTGTCCATGAAAGCCCTGCCT | | |
| | | TAATTGAGGAGNTAGCTCTGGGATCTCTGGGCTNGATCTTTGCACTGACTCTGCACAGACACACGCATCCTCTGAAC | | |
| | | CTCTATCTACTCTTTGCATTTACACTGTCAGAATCCCTGGCCGAGGCAGCTGNNGATACCTTCTATGATGTATATCT | | |
| | | GGTTCTGCAAGCGTTTATAATGACTACTGCAGTCTTTCTTGGCTTGACTGCCTATACTCTACAATCAAAGAGAGATT | | |
| | | TCACCAAATTCGGAGCAGGGTNGATAGCTGGNNATGAGGATNNTGAGCTTGGCAGGATTCTTGAAGCTGANTTTTT | | |
| | | ACAGAGAGACGATGGAGCTGGNCTTGGCCTCTCTAGGCGCCCTCCTCTTCTGTGGGNTCATCATCTATGATACACAC | | |
| | | TCGCTGATGCACAGACTCTCTCCCGAAGAGTACGTGAACGCTGNCATCAGTCTCTACATGGATATCATCAACCTCTT | | |
| | | NCTGCACCTGGTGAAGTTTCTGGAAGCAGNTAAATAAAAAGTAACCGAGCAGTNGTTCANAGACAGGTCTATTATGA | | |
| | | AAGGANGCTTTGGAATTNAACTTTAAATGGTTAATAATTAAANGCCAAATGTGAACTT | | |
| | BGH | TTTTTTTTTTTTTTTTTTTTAAAGATCACATATGCTTTTAATATTAACATTTAAGTTTAATTCAAGCGTCTTTCATAAT | | |
| | sequence | AGAACTGTCTCTGAACACTGCTCGGNTACTTTTTATTAACTGCTTCCAGAAACTTCAACAGGTGCAGGAAGAGGTTG | | |
| | | ATGATATCCATGTAGAGACTGATGGCAGCGATCACGTACTCTTCGGGAGAGAGTCTGTGCATCAGCGAGTGTGTAT | | |
| | | CATAGATGATGAACCCACAGAAGAGGAGGGCGCCTAGAGAGGCCAAGACCAGCTCCATCGTCTCACTGTAAAAAAAC | | |
| | | AGCTTNAAGAATCCTGCCAAGCACAAAATCCACAAACCAGNAAACAACCCTGCTCCGAATTTGGTGAAATCTCTCTTT | | |
| | | GATTGTAGAGTATAGGCAGTCAAGCCAAGAAAGACTGCAGTAGTCATTATAAACGCTTGCAGAACCAGATATACNTC | | |
| | | ATAGAAGGTAACAACAGCTGCCACGGCCAGGGATTCTGACAGTGTAAATGCAAAGAGTAGATAGAGGTTCAGAGGA | | |
| | | TGCGTGTGTCTGTGCAGAGTCAGNGCAAAGATCAAGCCCAGAGATCCCAGAGCAAACACCACANTTAAGGCAGGGC | | |
| | | TTTCATGGACANATGTCCGCAGAGCTTGGAAATACAGGAACAGGNCAGAGGTCACTGTAGTCAGGAGGACTTGCAG | | |
| | | AGAGAGGATACTGTAGACTTTTCTGAGAAAGGCCATGCGGATGTGCACGCTGGCCGACGCCACGCAGCTGCCGTAG | | |
| | | TTGAAGA | | |
| | Total | Human sequences: | | |
| | cDNA, | | | |
| | EST cluster | LOCUS AF113127 1325 bp mRNA PRI Dec. 4, 1999 | | |
| | | DEFINITION Homo sapiens S1R protein (S1R) mRNA, complete cds. | | |
| | | ACCESSION AF113127 | | |
| | | (human Cowpox virus homolog) | | |
| | | LOCUS AF151877 1325 bp mRNA PRI May 18, 2000 | | |
| | | DEFINITION Homo sapiens CGI-119 protein mRNA, complete cds> | | |
| | | ACCESSION AF151877 | | |
| | Motifs | | | |
| Clone # | T7 sequence | AGAAGAAAAGGCCGAAAAGGCCAAAATTAATAAGGCCATTCAGAAGGGCAACATGGAAGTTGCGAGGATTCACGCC | | |
| 92 | | GAAAATGCCATCCGCCAGAAGAATCAAGGGGTGAACTTCTTGAGAATGAGTGCACGAGTGGATGCGGTGGCGGCCC | | |
| | | GTGTCCAGACTGCAGTGACGATGGGCAAAGTGACCAAGTCCATGGCGGGTGTGGTTAAGTCGATGGACGGGACGT | | |
| | | TGAAAAGTATGAATCTGGAGAAGATCTCCGCTTTGATGGACAAATTCGAACACCAGTTCGAGACTCTGGACGTCCAG | | |
| | | ACGCAGCAAATGGAAGAGACAATGAGCAGCACGACGACGCTGACCACTCCCCAGAACCAGGTGGATATGCTGCTCC | | |
| | | AGGAAATGGCAGATGAGGCGGGCCTCGATCTCAACATGGAGCTGCCTCAGGGCCAGACCGGTTCCGTGGGAACGA | | |
| | | GCGTGGCTTCGGCTGAGCAAGATGAACTGTCCCAGAGACTGGCCCGCCTTCGGGATCAAGTCTGACGGNCGAACCA | | |
| | | GTCCGAGATTTCCCTTTGACGTGCTCTCTGGGTTTTAGAGAGATGTCCNAGAATTGTGCCAGAATGCCGAACGGTC | | |
| | | TTCTAAGAACCTACAGCCCTNACCAGATANGCTGAGAAATTCCAGTTTTCTCCACTTCTAACTGGATTTTAAAGCTCT | | |
| | | TTCTAGCTTATGATGATGTGTATTTTTTATAGCTGCCTTTTAACAGAACTGGTTCATTNCTCTACATAAATCTAGGAA | | |
| | | AAAATCGACNGACTNTAGCCCTCATGNNTTCCGTTTTTAC | | |
| | BGH | n.d. | | |
| | sequence | | | |
| | Total | UniGene: Mm.86545 | | |
| | cDNA, | | | |
| | EST cluster | EST-Assembly (Contig): | | |
| | | AAGAGTATTTCCCACCGAAGAAGTTGAACCAAGTGTTTACATACTTGTTAGTACCCATTC | | |
| | | CTTCTCCTTTCATTTTAATGCACCGCTTGCCAGTCTCTGTACCATCAACCACACTA | | |
| | | AAAATAACTTCAGTCTGATAATTCTAAGTAAAAACGGAATACATGAGGGCTAGAGTTCTG | | |
| | | TCGATTTTTTCCTAGATTTATGTAGAGCAAATGAACCAGTTCTGTTAAAAGGCAGCTATA | | |
| | | AAAATACACATCATCATAAGCTAGAAAGAGCTTTAAAATCCAGTTAGAAGTGGAGAAAA | | |
| | | ACTGGAATTTCTCAGCGTTATCTGGTGAGGGCTGTAGGTTCTTAGAAGACCGTTCGGCAT | | |
| | | TCTGGCACAATTCTGGGACATCTCTCTAAAACACAGAGAGCACGTCAAAGGGAAATCTCG | | |
| | | GACTGGTTCGGCCGTCAGACTTGATCCCGAAGGCGGGCCAGTCTCTGGGACAGTTCAT | | |
| | | CTTGCTCAGCCGAAGCCACGCTCGTTCCCACGGAACCGGTCTGGCCCTGAGGCAGCTCC | | |
| | | ATGTTGAGATCGAGGCCCGCCTCATCTGCCATTTCCTGGAGCAGCATATCCACCTGGTTC | | |
| | | TGGGGAGTGGTCAGCGTCGTCGTGCTGCTCATTGTGTCTTCCATTTGCTGCGTCTGGACG | | |
| | | TCCAGAGTCTCGAACTGGTGTTCGAATTTGTCCATCAAAGCGGAGATCTTCTCCAGATTC | | |
| | | ATACTTTTCAACGTCGCGTCCATCGACTTACACCACACCCGCCATGGACTTGGTCACT | | |
| | | TTGCCCATCGTCACTGCAGTCTGGACACGGGCCGCCACCGCATCC | | |
| | Motifs | | | |
| Clone # | T7 sequence | AGCGGACGCGCCCAGNAGCCGNGCTGCAGAGAGNTNCATCGNGCGACCGCTGCCGCAGGCGCTTGCTCCGAGTAGC | | |
| 93 | | CCTGCGGCTCCGCTTCTGCCATGANGATCCACGGCTTTCAGAGCAGCCACCAGGACTTCTCCTTCGGGCCTTGGAA | | |
| | | GCTGACGGCGTCCAAGACCNACATCATGAAGTCTGCGNATGTGGAAAAGTTAGCTGACGAGCTGNACATGCCATCC | | |
| | | CTCCCTGAAATGATGTTNGGAGACAACGTTCTAAGGATCCAGCATGGCTCTGGCTTTGNAATAGAGTTCAATGCTAC | | |
| | | GGACGCACTGAGATGNGTGAACAACTATCAGNGCANGCTCAAAGTAGCTTGNGCTGAAGAGAGGCAGNAAAGTAGG | | |
| | | ANGGAGGGCGAACACT | | |
| | BGH | TTTTTTTTTTTTTTTAAAATGGCATATGTCATATTTAGAAATGGCCAAGGACACACCTGNNTACTCNTCACAGGGATA | | |
| | sequence | CACTAATACAATCTTGAGATGCACACTCCTCCTCCCCACCGNCCACCTCTTTAATTTTTTTTAAAAAGNCNNTTATTTT | | |
| | | ATTTTAATTCTCCACAATTTTTAACAATGAATTATCAANATAAGNAATCTAACANTACTCAGAGACAAAGACATTTAAA | | |
| | | TGAAAAGATTTAAAAANTACTTGTGTGTACANACACACACACTNTGTTGCCCAGAAACTCCAACTAATGACTAATACA | | |
| | | AGATTAATTTTGTAGGTNACATGTNACANCTCTCCGTAAGTGCGAACAAACACCANAAAGCCCACAGCCAAGAGCCA | | |
| | | GAGTAGCACATNAGCATGCAGTCAGCATCCTATGGCGNCCAGCAGGTCCCAGGCATCGGNGCTCAGTGACAGCCTT | | |
| | | ACAGTCAGCCTCCTCACAGTGAAGACGATGCTTNAGGCCGGNTGCAGACCATGTCCCCTCATGAGACCCANACACAT | | |
| | | CACTGCTGACGTAAGCTCCAGAGCACATCTCTCTCTGNAGCAGGAACTTGAAAACAGACAGGATAAANAGGAGTTGT | | |
| | | GTGGAACCTGCCTGCAGCAATCTTTTATTNTCCNTTANGGCTAGAGNAANATCTGCGCATANGCN | | |
| | Total | from human genomic DNA (PAC 69E11 on chromosome 1q23-24): | | |
| | cDNA, | | | |
| | EST cluster | gene complement(16215 . . . 37678) | | |
| | | /gene = “dJ69E11.3 ” | | |
| | | CDS complement(join(16565 . . . 16708,17630 . . . 17692,19969 . . . 20064, | | |
| | | 25111 . . . 25242,31733 . . . 31832,32530 . . . 32709,37430 . . . 37533)) | | |
| | | /gene = “dJ69E11.3” | | |
| | | /note = “match: proteins 012199 P34274” | | |
| | | /codon_start = 1 | | |
| | | /evidence = experimental | | |
| | | /product = “dJ69E11.3 (Yeast YPR037W and worm C02C2.6 predicted proteins LIKE)” | | |
| | | /protein_id = “CAA16171.1” | | |
| | | /db_xref = “GI:4165247” | | |
| | | /db_xref = “SPTREMBL:O75663” | | |
| | | /translation = “MMIHGFQSSHRDFCFGPWKLTASKTHIMKSADVEKLADELHMPS | | |
| | | LPEMMFGDNVLRIQHGSGFGIEFNATDALRCVNNYQGMLKVACAEEWQESRTEGEHSK | | |
| | | EVIKPYDWTYTTDYKGTLLGESLKLKWPTTDHIDTEKLKAREQIKFFEEVLLFEDEL | | |
| | | HDHGVSSLSVKIRVMPSSFFLLLRFFLRIDGVLIRMNDTRLYHEADKTYMLREYTSRE | | |
| | | SKISSLMHVPPSLFTEPNEISQYLPIKEAVCEKLIFPERIDPNPADSQKSTQVE” | | |
| | Motifs | | | |
| Clone # | T7 sequence | GGGCCGCAGCAGGCGCGAGCGCCCAGGACCTGCCAGCTGAGCCTTCCGCCGTCGCCATGGGACAGAACGANCTGAN | | |
| 94 | | GGGCACGGCCGAGGACTTCGCCGACNANTTCCTTCGAGTCACNAAGCAGTACCTGCCTCATGTGGCGCGCCTCTGC | | |
| | | CTGATCAGCACCTTNCTGGAGGATGGCATCCGCATGTGGNTCCAGAGGAGNGAGCAGTGTGACTATATCGACACCA | | |
| | | CCTGGNGCTGTGNCTACCTGNTGGCCTCATCCTTCGCGTACCTCAACCTGCTGCNACAAT | | |
| | BGH | TTTTTTTTTTNTTTTTCCAAAGATTTTATCTCCAATTTTCAGGTGTCTGCAAATAAAGTTCTCAAAACATCTGNGCCT | | |
| | sequence | TTACCAAGGNAGGGGAAGGGAGAAGGACACACAAACGCCGGCAGNTTGNTGACTTTGCCCTGAACCAGGCCAGGGC | | |
| | | CCTGCGCCTCANCCAGGGAGGNNAGGNATCAGNGNGTTACCACTCTTTTTTCTTCTCATNCATGGACACACCCNCTG | | |
| | | NTCCAAGAGCCACCATCAGGAGCAGGCCTCCAATCACTGACATGGTCTGNAAGAAGTCGTATTTCAGGAAGTNNTGC | | |
| | | NTGNATTTATACACCGGGATNGACTCANANGGCGTNGAANNACACNCTGNTGNCAANNCAGCCNGNNTACTGNGAGN | | |
| | | CNCNAGNTGCCNGCNTNNCTCTACAANTNNANGGNNACTATCNNCNNCNCNNNNTNCCCCCCTNNCTNNNTCTNNNCNC | | |
| | | CNNNCTNTTCNTTCTANCNCACCATNNNNCCN | | |
| | Total | LOCUS NM_011512 2781 bp mRNA ROD Jan. 25, 2000 | | |
| | cDNA, | DEFINITION Mus musculus surfeit gene 4 (Surf4), mRNA. | | |
| | EST cluster | ACCESSION NM 011512 | | |
| | Motifs | | | |
| Clone # | T7 sequence | AGCTTCTTCAGNCGGNACGGGGTCAGCGAGCGGNTGCTTCGTGGAGCAGAGAGGTGCATNACNAGGTTCCCGATG | | |
| 95 | | AACCCAGAGAACNCTCCACCGTATCCGGGCCCCGGGCCAACAGCCCCATACCCACC1TATCCANAACAGCCAAAGGG | | |
| | | GCCAATGGGGCCTATGGGAGCCCCACCTCCTCAGGGGTACCCCTACCCACCANCTCAGGGGTACCCCTATCAAGGAT | | |
| | | ACCCANAGTACGGCTGGCAGGGNGGACCTCAGGAGCCTCCTAAGACCACAGNGTATGTGGNGGAAGACCAAAGAAG | | |
| | | AGACGACCTGGGCCCATCCACCTGCCTCACAGCCTGCTGNACTGCTCTGTGNNGCTGCTGCCTCTGGGACATGCTCA | | |
| | | CCTGATCANCTGATGAGCCCAGCTCTTCCGCTTGGCCGCTCTGTGCCACCTCCGATAAGNGTGCCNGGCCCCATCTC | | |
| | | TTCTGATNGCTATAAAGNGGCTAGCTCTGCGNAGACACCTCTACTTTCTGTCCTA | | |
| | BGH | TTTTTTTTTTTTTTTTTTGATTTTGGACAGATTTATTGAAACATAAAGGGTATGAGCAGAGAGATCTAGNAGNGTGT | | |
| | sequence | CACATATTGCCATTACCTTGAGTGTATAATTTAAACATTATAAATATATATTTCATAACTAAGCCTTTGGCCAAAAAA | | |
| | | GTAAATTATTTAGCACATTTTTTAAAGATCAGTAAGAAATGAGTTTTGAACATTAAAAAGATCAAGTCACTGAACTAA | | |
| | | ATAGCAGTAACCCTCACTAATCTAAAACTCCATAGGACAGAAAGTAGAGGTGTCTGCGCAGAGCTAGCCACTTTATA | | |
| | | GCAATCAGAAGAGATGGGGCCAGGCACACTTATCGGAGGTGGCACAGAGCGGCCAAGCGGAAGAGCTGGGCTCAT | | |
| | | CAGGNGATCAGGTGAGCATGTCCCAGAGGCAGNAGCAACACAGAGCAGTCCAGCAGGCTGTGAGGCAGGTGNATG | | |
| | | GGCCCAGGNCGCCTCTTCTTTGNTCTTCCACCACATACACTGCGGTCTTAGGAGGCTCCTGAGGNCCACCCTGCCAG | | |
| | | CCGNACTGAGGNTATCCTTGANAGGGGTNCCCCCTGAGGTGGTGGAA | | |
| | Total | Mouse UniGene: Mm.27841 | | |
| | cDNA, | Human UniGene: Hs.100132 (human homolog) | | |
| | EST cluster | | | |
| | | EST cluster: Mm27841 | | |
| | | TTTTATACTAAAAAATGTGAGTCCCAAATTCCGCATTTGTACAACAACGAGGGGGAGCGC | | |
| | | TCAGACCCCTACCCTAAACGGGACCGCACCCCCACCGAACGAGAAGAGGAGCTTGCGCTC | | |
| | | CATCCCTGAGGGTCAACTTATGGCCAGTTCACCGTAACCGCTGCAGATACCTCCAGAGTA | | |
| | | GTCGGATTTTGGACAGATTTATTGAAACATAAAGGGTAT | | |
| | | GAGCAGAGAGATCTAGTAGTGTGTCACATATTGCCATTACCTTGAGTGTATAATTTAA | | |
| | | ACATTATAAATATATATTTCATAACTAAGCCTTTGGCCAAAAAAGTAAATT | | |
| | | ATTTAGCACATTTTTTAAAGATCAGTAAGAAATGAGTTTTGAACA | | |
| | | TTAAAAAGATCAAGTCACTGAACTAAATAGCAGTAACCCTCACTAATCTA | | |
| | | AAACTCCATAGGACAGAAAGTAGAGGTGTCTGCGCAGAGCTAGCCACTTTA | | |
| | | TAGCAATCAGAAGAGATGGGGCCAGGCACACTTATCGGAGGTGGCACAGAGCG | | |
| | | GCCAAGCGGAAGAGCTGGGCTCATCAGGTGATCAGGTGAGCATGTCCCAGAGGCA | | |
| | | GCAGCAACACAGAGCAGTCCAGCAGGCTGTGAGGCAGGTGGATGGGCCCAGGTCGT | | |
| | | CTCTTCTTTGGTCTTCCACCACATACACTGTGGTCTTAGGAGGCTCCTGAGGTCCACC | | |
| | | CTGCCAGCCGTACTGTGGGTATCCTTGATAGGGGTACCCCTGAGGTGGTGGGTAGGGGTA | | |
| | | CCCCTGAGGAGGTGGGGCTCCCATAGGCCCCATTGGCCCCATTGGCTGTTGTGGATA | | |
| | | AGGTGGGTATGGGGCTGTTGGCCCGGGGCCCGGATACGGTGGAGGGTTCTCTG | | |
| | | GGTTCATCGGGAACCTGGTGATGCACCTCTCTGCTCCACGAAGCAGCCGCTCGCTGAC | | |
| | | CCCGTCCCGACTGAAGAAGACTGGACCGGACTACGCCAACAGCGCCGCACCT | | |
| | | GTCGCGAGCCACTAGAGCCGCAGCGGCGCGCCTGACGTCACCCGAGCCTCCTGCGCT | | |
| | | GGAAGCCCGGAGTCACGCGATCTCCCTGGCAACGCGCGAGGGGGCGGCACCAAGCGAGGG | | |
| | | CGGTGTCATAGCGCGAGCCCGCCCCCAAAACTCTGCATCACGTTGGGGACTTAAGCCTGT | | |
| | | CCCTTAGCTCCTTGTGGTCCCCGATCTGTATCTATAGTTTCACTCCGGGTCCTTGCGAGC | | |
| | | ACCCGGCCCCACGTCGCTAGGACCGGAGATTGGGAAAAAGGGTAGGGGGGCTGAGGACCA | | |
| | | GCTTGGTACACATCATGTAGGGCTGCTGCTGCTGCTGCTGCCTGACCTCTTCTCCGCTGC | | |
| | | TAGGTTCGGGCCGAGGCCTGTTCCGA | | |
| | | Probable ORF: | | |
| | | SEQUENCE 104 AA; 11402 MW; A53B91320E342D54 CRC64. | | |
| | | MNPENPPPYP GPGPTAPYPP YPQQPMGPMG PMGAPPPQGY PYPPPQGYPY QGYPQYGWQG | | |
| | | GPQEPPKTTV YWEDQRRDD LGPSTCLTAC WTALCCCCLW DMLT | | |
| | | EST cluster: Hs.100132 (human homolog) | | |
| | | GTCAGGCGCCCGGTTGCATTCCGAACAGGCAATCTGAGACAGGTGCGGCAAGTCTACT | | |
| | | GCGGGCTGGTCCGGGCTCGTCAGGTTCAGACCCGACCGTTATCCAGTCGGTTCGTGGAGA | | |
| | | GGAGAGGTGCACTTTACAGGTCCCCGATGAACCAAGAGAACCCTCCACCATATCCAGGC | | |
| | | CCTGGTCCAACGGCCCCATACCCACCTTATCCACCACAACCAATGGGTCCAGGACCTATG | | |
| | | GGGGGACCCTACCCACCTCCTCAAGGGTACCCCTACCAAGGATACCCACAGTAC | | |
| | | GGCTGGCAGGGTGGACCTCAGGAGCCTCCTAAAACCACAGTGTATGTGG | | |
| | | TAGAAGACCAAAGAAGAGATGAGCTAGGACCATCCACCT | | |
| | | GCCTCACAGCCTGCTGGACGGCTGTCTGTTGCTGCTGTCTC | | |
| | | TGGGACATGCTCACCTGACCAGACCAGCCCAGCCGTCCTGTCC | | |
| | | TGCCAGCTCTGCTGCCACCTCTGACAGGTGTGCCTGCCCCCATC | | |
| | | TCTTCTGATTGCTGTTAACAAATGACTAGCTTTGCAC | | |
| | | AGACACCTCTACCTTCAGCACTATGGGATTCTAGATTAATGGGGGTTGC | | |
| | | TACTGTTTAATTCAGTGACTTGATCTTTTTAATGTCCAAAATCCATTTCTT | | |
| | | ATTGATCTTTAAAGATGTGCTAAATGACTTTTTTGGCCAAAGGCTTA | | |
| | | GTTGTGAAAAATATAATTTTTAAATTATACATTCAAGGTAGTGGCCAAATGTAACA | | |
| | | CATCAATCATGGAATGATTTCTCTGCTAACAGCCGCCTGTATGTTTCAATAA | | |
| | | ATTTGTCCAAAGCTCAAAAAAAAAAAAAAAAAAAAAAAAAAAAAAAAAAAAAAAAA | | |
| | | Probable ORF: | | |
| | | SEQUENCE 97 AA; 10631 MW, 93A64A2482B41F58 CRC64. | | |
| | | MNQENPPPYP GPGPTAPYPP YPPQPMGPGP MGGPYPPPQG YPYQGYPQYG WQGGPQEPPK | | |
| | | TTVYVVEDQR RDELGPSTCL TACWTALCCC CLWDMLT | | |
| | Motifs | proline-rich | | |
| Clone # | T7 sequence | GNGTGGCCCTGCCTCGCTGGGGCCTGACCTGGTNTCCCAACCTGGAGNCCAGAAGGNGGCTTTCTGCGGAGCCGN | | |
| 96 | | GGAGGAAGGACGTGNTCTGCGACGGANTCTAGCAGGCAGGGGANTTGAGCTGGGCCTGGCCCTGGGCACAGAGTC | | |
| | | ACNGNAGCTATNGANGGACTCCGCAGATGNGGACTCAGCTGAGGAGGGNGNNGTGGANGCCGNCANCTNAGACNCN | | |
| | | ANCCTCN | | |
| | BGH | TTTTTTTTTTTTTAAAGTCACTCAGTTTATTANAAGCCATGGGAAATGTGAGAGAAACGTTCCAAGCACTTGNTGCT | | |
| | sequence | CCTGAGCATCAAAGATGGAGTGGGNGGCTTCGGCATCAGTCCCCCATTGGCAGCCACAGGGCCTTTGNTCGNGCTG | | |
| | | CGTGAAGACTCANNTCCTGTCCTGCCCCCTGGACCTCCACATCCCAGGCCCTTGGGAAGCCCCTGTTTACCNACACA | | |
| | | GACTTGAGGTTTCCTGCAGAGGCCCATTCCACAAACTGGGAGCCCTGGGCCGAGCCGAAGTACCACAGGGCTTGGA | | |
| | | CATCCTGATGTAAGGCCAGACAGCGGNTCAGGNGGNCGCGATCCCCTGTCACTACACTCACCAGGCCAGCAGGAAA | | |
| | | CAGAGGAGCTATATCCTGGCAGACCTCCAAGGCCAGCAGAGGACATGCCCCACTGGGTACTAAGACCACGGCATTG | | |
| | | CCATGGNCCAGTGCAGGGGCCAGTAGTGACACAAAAGCCAGCAGGGGCCACTCATCCGGGCACACNANGGNCAGCA | | |
| | | CTCCCAATGGTTCTCGAAGCCGNAGCACAGGGCCTGTNAATCCTGTTACCTGAAGTGTCTGGCCTTGGTCCTGAACC | | |
| | | CGNTGCCCCATGTNTGNAGTCGCCTTANACTCAGTTTACTTCAATCTTGGCNACTTTAGGCGCTGTCCCGTGCCTTC | | |
| | | TAGTTGTGAGGNNAGCACTGGCTTCCTGCNTTCAGAGCAGCCNNNGNGCCCAAGNNNNCT | | |
| | Total | UniGene: Hs.133494 | | |
| | cDNA, | Mm.31778 | | |
| | EST cluster | Rn.11778 | | |
| | Motifs | | | |
| Clone # | T7 sequence | AGATGGAGCAGNGTTTCCAACANGGACGACATNAAGACCTCACTCAAGAAAGTTGTGAAGGAGACATCGTATGAGA | | |
| 97 | | TGATGATGCAGTGTGTATCGCGAATGCTGGCCCATCCCTTACACGTGATCTCGATGCGATGCATGGNGCAGNTTGN | | |
| | | GGNACGGGAGGCCAAGTACAGAGGNGTGCTGAGTTCTATNGGGAAGATCTTCAAGGAAGAGGGGCTGCTGGGATT | | |
| | | CTTCGNTGGCTTAATCCCTCACCTCCTGGGCGANGAGGNTTTCTTGNGGNGCTGTAACCTGCTGGCCCACTTCATNA | | |
| | | ATGCCTACTTGGTGGACGACAGCGTGAGTGACACCCCAGGGGGNCTGGNAAACGACCAGAATCNAGGTTCCCAGTT | | |
| | | TAGCCAGGCCCTGGCCATCCGGAGCTACACCAAGTTTGCGATGGNGATNGCAGCGAGCATGCTGACCTACCCCCTT | | |
| | | TCTGCTCGCTGNNGATCTCATGGCAGT | | |
| | BGH | TTTTTTTTTTTTTTACCACCAATACATTTATTCNNGGAGATGGGTCTATCTTACCACGAGGGGAGGACTAGATGTCG | | |
| | sequence | NTCTATGNAACCTGTGCGTATTCGCACCCAGCACAGTGACTGAACCCTCACACCTGGCGTCACCAGCACAGACAAGC | | |
| | | AGATNAGGNNATGGTCTGAGGAGAACATGATTTCCTATTCAGGAGAAGGCACCACCCTTGTATAAGAAAATTAGTGT | | |
| | | TGNGAACATAGCGCCAGNCTCCCATGGCCCAGGTGTGATGGCGCCCAATTTACAAAGCAGGAAGTGGGNNGCGGCG | | |
| | | GTGCTTCTGGCTGACTGGCAGGATGAGCTGNGCTAGAGGNGCAGGGAAGCCTTGCCACTGAGTGACGTTTGCCTC | | |
| | | TGCAGCCTGCCTCTGCCTGAGTACAAGATGGACTCCAGTACCTCTAGGCAGNAAGGGNATGCCACCCCACCACTGNT | | |
| | | CCCCCAGGACTTCCCCAGGTCCCAGGTGACCTACNTCCACNAGCCCANATNTGNACAGACNCATNNCCAAATATGGACA | | |
| | | TGAAGCCAGCNTGGNCTTGNAGTCACCCTTGCNCATCTGANATGATGGGCCCTTCCCNGNGTTAGNGCTAN | | |
| | Total | ATTENTION: Clone incomplete, first 647bp 5′ are missing (about 180aa) | | |
| | cDNA, | | | |
| | EST cluster | | | |
| | | LOCUS | AF192558 1928 bp mRNA | ROD Nov. 26, 1999 |
| | | DEFINITION Mus musculus domesticus mitochondrial carrier homolog 1 isoform b (Mich1) mRNA, complete cds; nuclear gene for mitochondrial product. | | |
| | | ACCESSION AF192558 | | |
| | | Human sequence: | | |
| | | LOCUS AF192559 1941 bp mRNA PRI Feb. 17, 2000 | | |
| | | DEFINITION Homo sapiens mitochondrial carrier homolog 1 isoform b (MTCH1) mRNA, partial cds; nuclear gene for mitochondrial product. | | |
| | | ACCESSION AF192559 | | |
| | Motifs | | | |
| Clone # | T7 sequence | AGGGCGGAGGAAGCGGACTGTTCCGGAGCTCTGCCTAGCCGGGCCCAACCTT2GCTCCAGAGATCATGGCTGCCGA | | |
| 98 | | GGATGTGGTGGCGACTGGCGCCGACCCTGAGCGAGCTAGAGGGCGGCGGGCTGCTGCACGAGATTTTCACGTCCT | | |
| | | CCTCTCAACCCTGCTCCTCCTGGGCCTCTGCNTCTTCCTGCTCTACAAGATCGTTCCGCGGGGACCAGCCCGGTGCT | | |
| | | AGTGGCGACAACGACGATGANGAACCACCCCGCTGCCCCGCCTCAAGNGGCGCGACTTCACCCCTGCCGAGCTGAG | | |
| | | GCGTTTNGATGGNGTCAGGACCCGCGCNTTCTCATGGCCATCAACGGTAAGGTGTTCGACGTGACCAAAGGCCGCA | | |
| | | NGTTCTACGGGCCTGAGGGGCCNTATGGGGTCTTTGCCGGAAGAGATGCATCAGGGGCCTTGCNCATTTTGCCTG | | |
| | | GANANAGAAGCACTGAAGGATGANTNTGACGACCTTTCTGNCCTCACCCTGCACAGCAGGAGANCCTGANTGACTGG | | |
| | | NACTCTCANTTCACTTTCNAGTNTCNTCACNTGGGAAAACTGCTGAAGGAAGGGGAGGAGCCTACTGTGTACTCAGA | | |
| | | TGATGAAGANCANAAGATGAGACAGCTTGGANGANTGANTGNNNCATTCGGTGGAGCNTATCTATNTTTGTATTTTG | | |
| | | CANCNNTCNTTTTGTANCNTTCNNGTCTGCNTTNCNACATGGNGATTTNNNTATTANACAGTTTTGCNCTTGCTGA | | |
| | BGH | n.d. | | |
| | sequence | | | |
| | Total | LOCUS NM_016783 1786 bp mRNA | ROD Jun. 15, 2000 | |
| | cDNA, | DEFINITION Mus musculus progesterone receptor membrane component | | |
| | EST cluster | (Pgrmc-pending), mRNA. | | |
| | | ACCESSION NM_016783 | | |
| | Motifs | | | |
| Clone # | T7 sequence | AGCTCGAAAGCGACATGGCGGTTCTCTTAAAGCTGGGCGTTCTCTGCAGTGGCCAAGGAGCTCGAGCTCTCCTACT | | |
| 99 | | CCGAAGCCGGGTGGTCAGACCCGCTTATGTGTCAGCATTTCTCCAGGACCAGCCTACCCAAGGACGGTGTGGTACC | | |
| | | CAGCACATTCACCTGTCACCAAGCCACCACTCTGGTTCCAAGGCTGCATCTCTCCACTGGACCAGTGAGAGGGTTGT | | |
| | | CAGNGTTCTGCTCTTGGGGCTGATCCCTGCTGGGTACTTGAATCCCTGCTCTGNGGNGGACTACTCTCTGGCTGCA | | |
| | | GCCCTCACCCTGCACAGTCACTGGGGCCTTGGACAAGTGGTTACCGACTACGTTCATGGGGACACCCTGCCGAAGG | | |
| | | CTGCCAGGGCAGGCCTCTTGGCACTCTCAGCTTTGACCTTTGCTGGGCTTTGCTACTTCAATTACCACGATGTCGGC | | |
| | | ATCTGCAGAGCGGNTGCCATGCTGTGGAAGCTCTGACCTGGGTGCAGCACTTTGATTGTGTGCCTCCTTGCCTCTG | | |
| | | CTTTACCAATGCCGTTCACCTCGCAGTGAGGGGGGATGAAGGATAAGCCCATTGGTGGGCAGAATGTCTTCTAATT | | |
| | | ACATGGTTATTTTCAGAATTTATTTGTTGAGGAAGAGGTTTGAGGAGTTAGGTTCGACCATTCGTGAGTCTGTGTT | | |
| | | CCATACTCCACTGAGTGTGGGCACTAGCTCACAGCCTCGCGGTGAGACTGAACATTTCATGAGCTCATGGTGCCTTT | | |
| | | GACCACCATTTTCTTAAGGAGAGCCAGCTGANTGCTGTCAGGATAAGAGCATCTCTTCA | | |
| | BGH | n.d. | | |
| | sequence | | | |
| | Total | Human sequence: | | |
| | CDNA, | | | |
| | EST cluster | LOCUS NM_003002 1313 bp mRNA FRI Mar. 19, 1999, | | |
| | | DEFINITION Homo sapiens succinate dehydrogenase complex, subunit D, integral membrane protein (SDHD) mRNA. | | |
| | | ACCESSION NM_003002 | | |
| | | Motifs | | |
| Clone # | T7 sequence | AGCTAGGATCTTTAGCTTCAACTCCTACTGCTNCTTCTAACCCAGCAGCCCCGGATAATGCAGCCCAGGAGGAGCTC | | |
| 100 | | ATGATCACCCTGATCACAGGATTGGCGTCCCTCACGTCGAGAACCTCCATGGGCATCATCGGTGGNGGGGGCCGGT | | |
| | | AATTTNGAAAACAGGTGGGNTTGNAACCTAATCTTTGCNNCTTAAGGAATGACCGGGGCTTNGACCTTTAATNA | | |
| | BGH | nd. | | |
| | sequence | | | |
| | Total | ATTENTION: Clone incomplete, first 900bp 5′ are missing (at least about 300aa) | | |
| | cDNA, | | | |
| | EST cluster | Human sequence: | | |
| | | LOCUS HSU95822 2176 bp mRNA PRI Jul. 30, 1997 | | |
| | | DEFINITION Human putative transmembrane GTPase mRNA, partial cds. | | |
| | | ACCESSION U95822 | | |
| | Motifs | | | |
| Clone # | T7 sequence | AGCTGCGCCCNTGTACCCTAGGTCTAGAGTGNACCCGCCCCGGGAAAGAAGCTAGGCCGGGTCCGCAGCATCAGCC | | |
| 101 | | ATCACTTCGCACAAAGCAGCGCAGCTCCGGGACCGCGGAGGACGACAGCGGCGCGGCAGCGGCGCGGCGACACTCA | | |
| | | GTGCACCGTATGCCCCTGCGCCCCTGCCGAGGCAACCGGAGCGCCCCGAGAGACCGCGCCGCCGCGGGGTCCAGG | | |
| | | TGCAGTTAGCGAGCCTAGCCCGCAGCGCGCAGTCGCGGGAGAGCGGGGAGCGGCAAGCAACAGGGAGCGGGACG | | |
| | | GCGGCGAGGCGCTCGCGGGCCCCTCCTGCTGCCCGCGCCCGGCGAGCTCATGGCGGCCATCCGCAAGAAGCTGGN | | |
| | | GGNGGNGGGCGACGGCGCGTGCGGCAAGACGTGCCTGCTGATCGTGTTCAGTAAAGACGAATTCCCCGAGGTGTA | | |
| | | CGTGCCCACCGTGTTCGAGAACTATGTGGCGGACATCGAGGTGGACGGCAAGCAGGTGGAGCTGGCGCTGNGGGA | | |
| | | CACGGCAGGCCAGGAGGACTACGATCGTTTACGGCCGCTCTCCTATCCGGACACCGACGTCATCCTTATGTGCTTCT | | |
| | | CGGTGGACAGCCCGGACTCTCTCGAGAACATCCCCGAGAAGTGGGTGCCCGAGGTAAAGCACTTCTGCCCCAATGT | | |
| | | GCCCATCATCCTGGTGGCCAACAAAAAAGACCTGNTCAGCGACNAGCATGTCCGACGGAGCTGGCCCGCATGAAGC | | |
| | | AGGAGCCAGTNCNGCACGGATTGACGGNCGCTCC | | |
| | BGH | n.d. | | |
| | sequence | | | |
| | Total | LOCUS MMRHOBGEN 4612 bp DNA ROD Feb. 3, 1997 | | |
| | cDNA, | DEFINITION M. musculus rhoB gene. | | |
| | EST cluster | ACCESSION X99963 | | |
| | Motifs | | | |
| Clone # | T7 sequence | AGGTTTCCTCGCCGCCGGCCAAGATGAACCGNTTCTTCGGAAAAGCGAAACCCAAGGCTCCGCCACCTAGCTTGACG | | |
| 102 | | GACTGCATTGGGACGGNGGATAGCAGGGCAGAATCCATTGACAAAAAGATTTCCCGGCTGGATGCTGAACTAGTGA | | |
| | | AATATAAGGATCAAATCAAGAAGATGAGAGAGGGTCCTGCTAAGAACATGGTCAAACAGAAAGCCCTGAGAGTTTTA | | |
| | | AAGCAAAAGCGGATGTATGAGCAACAGCGAGACAACCTGGCCCAACAGTCCTTTAACATGGAGCAAGCTAATTACAC | | |
| | | CATCCAGTCACTAAAGGACACCAAGACCACGGTTGATGCCATGAAGTTGGGAGTAAAGGAAATGAAGAAGGCATAT | | |
| | | AAGGAAGTAAAAATTGACCAGATTGAGGACTTACAAGACCAGCTGGAGGATATGATGGAAGATGCAAATGAGATCC | | |
| | | AGGAAGCCCTGGGCCGCAGCTACGGCACCCCAGAGTTAGATGAGGACGACCTGGAAGCAGAGTTAGATGCGCTGG | | |
| | | GCGATGAGCTTCTGGCTGATGAAGATAGCTCCTACTTGGATGAGGCAGCTTCCGCTCCTGCAATTCCGGAAGGTGT | | |
| | | TCCCACTGACACAAAAAACAAGGATGGCCGTGCTGNNGGATGAATTTGNACTGCCGCAGATTCCCGCTTCGTAGACT | | |
| | | TACAACATTCAGCACGTGATGTGAAACAACANGAGAAGTATTCTGGNACTAGNAAATAGTTCCCCGATCTGCCAACC | | |
| | | AGATTAGGNTTCTTTCCTTTCTTTGAT | | |
| | BGH | TTTTTTTTTTTTTTTTTTTTTTTTTTTTTTTTTTCCTTTTGAAAAATATTNATTGAGTATAAANAAATGNCCTATNCAT | | |
| | sequence | TAAATATCAAGAAATATTCATTATTCATTACAAGTCAGGCTAGTATACTGAGGGAACCATGGAATCAAAATAGTANCT | | |
| | | CATGATACCGAAGAGACAGTTCATTCCAACAGTANATCGTGNAATGACTTGCTCACATGACACAACAAAATNTATCAA | | |
| | | AAGAGGCCATGACTTAAACCCATNTACAATACCACAACAGACAAGGGTTCTGAAGCATACTATCAGCAGTCCCAATCT | | |
| | | CATCAATCACTTTTATCTTATCAATGTATCAGCNCACTGAGACTGTACTTACNCTACCATAGAATTCAGAGTCTCATC | | |
| | | TCCTTGGTTTAATTTCAGATTATTGTTAATGAATGAAAACAAGTCATACAAAGTCAAGGGTACTTTTCAATTAGTCTT | | |
| | | TCCTAAGAGAAAAAAAAAAAAANGAATCAAGTTTTAGTAAAGACAGTTTCCCAAAAGCAATGAATTCCTTANCGGAAA | | |
| | | AAATAAAAATNCNGGAGCAGTGTANANAGCTTTTTCTTCAAAGAAAGGAAAGAAACCTAAATCTGGTTGGCAGATCG | | |
| | | GGAACTATTTCCTAGTCCCAGAATACTTCTCTTGNTGTTTCACATNACGGGCTGGAATGTTNTANGGTTTACGAAGC | | |
| | | GGGAAATTTGNGGNAGNCCAAATTCATCCCCCAGCACGCCATNCTTGTTTA | | |
| | Total | Human sequence: | | |
| | cDNA, | | | |
| | EST cluster | LOCUS AF161525 1197 bp mRNA PRI Feb. 1, 2000 | | |
| | | DEFINITION Homo sapiens HSPCI77 mRNA, complete cds. | | |
| | | ACCESSION AF161525 | | |
| | Comments: | The gene contains a BH3 domain which occurs in many Bcl-2-like genes. Genes | | |
| | | with such BH3 only domains are proapoptotic and have the evolutionary | | |
| | | conserved function to induce apoptosis. Our experiments show that CGI-135 can | | |
| | | interact with Bcl-2 gene family members. The BH3 domain is necessary for | | |
| | | efficient induction of apoptosis by CGI-135. | | |
| | Clone | | | |
| | Name, | | | |
| | Designation | | | |
| | (Gene, database) | | | |
| Clone # | T7 | AAANCTNNATCTCATCTACACNNGCNNTNGATCCACTANNAACGGGNCGCCAGAGACGCTGNAAAGGTCCGCTCCTNC | | |
| 103 | sequence | GCTGNAGAGNACGTGCCCTGANCTCNNNNGACAAGGCTTCCNTTNTANCCGNCANCNTTTANNANTCCNTCNTANTNCT | | |
| | | CNCCTNCNNNATTNTTATNTNNTACTCNCNNNCTNCTCCCTCTNTNCNTNTCTCTNCNTNNNCCTACTCTTCCTNTNNCC | | |
| | | CCTTCCCTNNTTATNNNTCTTTATCCACTCCNTCTCNNANTATNTNNTNNTNNCAAATCTATNTCTNTNNCCNNNNCNCC | | |
| | | CANNNNTCTNNNACCNTNCCTCNTTTCNNTTTNCCTACTCCCTANTTCCTCNTNCTTACCNCCNNCCATTNTCAATCNNN | | |
| | | CTNCTNTCCAACNCTCANCNNTATCTTTCCCCNNTCTNTNANCNATNCCTACTCNNTATTCNCCNCCTTCTNCCNCNACT | | |
| | | ANCTCNTCATTTNNCNNNAANCCNCANTNNTNNTNTNNCNNNACNNTNTNATTTCNTACANTNCCCNACNNTCCTNTTNN | | |
| | | NCCACTNNTTTCTCTANNNTACTCTTACNNAANNTCTNANNCTCCCNCNNNNCNTCTNTCTCCNTTCANATCTTNCNNNC | | |
| | | CNNATNNACCTNTCATCNCNCTNTCCTTNNCNANANNANNTCCNCNTTCNTCTTTCNTCACTANCTTANNCCNTNTTNTC | | |
| | | NCTCNTATTNNTTTANTTNTTTCCTCNTNTNTCNNNNTCTNTNNNCTATCACCTCTTCCTCNANTTCNACNCNTNTCTTN | | |
| | | CNNCATCTNNNTAANTNTTCTNANACTCTCCTATTANCCTNCCAACTCCCGTCCTCNTTCTCCCCCNCNTNCNNTCNCCN | | |
| | | TCNACTCCTTCAATNATATAATCCCTNCACCCNNNNCTNTANNCCTCNTTTATNTCTTCTTACCACNAATCAANTNAATT | | |
| | | CTTNTTCNATNCTNNCNCNCCTTTCAACNCNGCTATTTCACAACNNNCACNCNTTCNNNTNCTACNCTN | | |
| | | No homologous sequences in the databases | | |
| Clone # | T7 | TTATNAACCNCTNTCNCCACGCTTGGACCCACTANCAACGGNCGCCAGTGTGCNTGGAAAGNACGAANGGCTTATGCG | | |
| 104 | | CNAGGTNCCACNCNNNTTGGGNGNCNAANNNGNCNTNCNAGAGGNAGGGCCNNTTTTNNNNCGGNGNNCNNANNNNAA | | |
| | | GNCGAANGNGTCNNCNTANCNCNNTTNNNCNCCCACGCCCNNNNCNCNNTNNANNCCCNATNCNNCANNANNCTCNCTCT | | |
| | | NTTTTNCTCCCCNCNCCTNCTTCNNCTNCNTTATCNNCNNCTTTCNCCTCTCTNNCNTACNCTACCCTTCCTTCNTNNNC | | |
| | | NCCCCCNNNACTCNCCNCCANNCACTNNNCCANTCCTTCNTNNACCTATCCCCCCTCCCCNNCCTCACNCTACANTCTCC | | |
| | | TCCCTNTNCCNNCCCTTTCTTCTTCCANCCTCTTNCTCCNCCNTNNCCCCCTCCTCCTCTCNTNTATTCATNNTACNNTT | | |
| | | CATTCNNCNATTCNTTTNCNNACCTNNNTTCTNNCNTCCACANNNCTTATNCCCATCTCTTATNNNACTTCTNCNANTCT | | |
| | | CCTNNTCTCCTCTACNTNNANCTCTCNNNNTACNCTNTNTCTANACATCTCTCTCTCACTNTCNCTCACNCTATCTCTCC | | |
| | | TCNANTNCTCANTNATANTCTCCCACTCCTANNCNCTCTCCCCCCTCTCANTAGTTANCTCTNTCTCCTTCNCTCNTATA | | |
| | | GCNTATCNCAANNCTCTCCNTACNCTCNCCTCGTCATCTATCANCCTCTCACCTNTNTCANACTNTCATANATCNCTCCT | | |
| | | CTNCTCCCATNATNTTCCACCTCACTNACNTTNTNCNTNNTNCTTCNTATCCTCCNATCCNTTCCNTNCANNCCTCTCTC | | |
| | | CCTCTCAACCNNTCNCATCTCCCCCTCTTCNNTNNNNNTCANCNCNTCNCNCCATTNTNCNTCCACNTCACCNCTCCCTA | | |
| | | TNTNTTCTCTNNACTCANACCCCNACCATCCCCNANNTANCTNCCCCCTCANTNCAACNCNTCTNTCNNACTCCCTCTCN | | |
| | | CTCNACTCTNATCTNCTCNNNTCANTCACTNTCTCTNNTCTCTCCACCACCGTCTACTCATACTTNNTCTCNCCCTCTNT | | |
| | | CNTCCCNCNCTCNCATNCTCNTCTCATCNTCTCANTCCCTCTANNTNTNCATTTCTNNTCCNTCNTTTNCTCTACNCNCT | | |
| | | CCCGNACACACTNCTACCCTCCTCANTTNCTCCCACNNCTACCANCNCTNTCTCCTCNNTNCNNCNTNTACTNCTCGNNA | | |
| | | CNCCNNTNTNTNTCTCTGNTCTCCTNATCATCNNCTCNNACNCNTNTTCANATCNCCANNTATNTCTNCANCTACCC | | |
| | | >ug\|AW682500.30119\|Mm.30119 EST01349 Mus musculus cDNA | | |
| | | Length = 659 | | |
| | | Score = 34.2 bits (17), Expect = 1.2 | | |
| | | Identities = 21/23 (91%) | | |
| | | Strand = Plus/Minus | | |
| Clone # | T7 | TACNCACCNCTTGTACCGGCTCGGATCACTAGTAACGGCCGCCAGNGTGCTGGAAAGTCTTTCTGTACTTCCTGCCTG | | |
| 105 | | GCACCTGCTTTCCCTGCCTCTACTNGACCAAGAGTTACACAAGAANGCGTTCTCCCCCGCCAGCTCCCACTTGGAAGA | | |
| | | TTTNACGAGGNGNGNGGCTCCNNGGCGCCATGGCCACACCTACGCTNNNTGNTGATNNNCTANCGGAGGGCCCACCCC | | |
| | | CCTNNCTCCTTACCNCTCNTCTNNNNCCCCCCCCCCCNNTTTCNCCATNTTCNCNCCCCCCCTNCNTCNTCTTCTCCCCT | | |
| | | CNTTCTNCNTCNCCCATTCCNNCTNACNNNCNTCTNCATTCCNNCNNCCCTCTCTCTCNCNCCTCTCCCNTCTCCTNTAT | | |
| | | TCTTTCTTCACCCTCNCCTCCNANCCNTCCTCTCTCNTNCCTANTTCNNCCCCTNTNTCCNCATNCTCTAACTCNNCCTC | | |
| | | ACACTCTCCCCTCNATCCATCANNNCTCNCTNCCTCCTNTCTACAANTCNCNCNCCTCNNTNTCNCNCTANCTCNCTNCC | | |
| | | TCTNNNACNCNCTTCACCCNNTCTNTCNNNTTCCCTCNACTCCCCTCTCTCTCCTCCNTTNCTCTNNTCNNTTNCTNCCN | | |
| | | ATCCNCCNNCNCCCCTNANTCTCCNNCCNNTACCTTCCNCTTCCCATCCTNCTCCCTCNNNTCCCANTCTCCNATNCCNC | | |
| | | CTTCNNCTCNTCCCAACNNNCTATTNCNNNCNCCTCCNNTNNCTTCNCCTTCNCCTTNNATTTTTCNNTNTCTTTCCCTC | | |
| | | CTNCACNCNNCTCNTNCNNTTNNTNTCTTNNCNCNATNCCTTTNCCTCNNNNTCTCNCTCTCCNTCTNCNTCCTCCNCTC | | |
| | | TCNTCTCNNTTCNTCATCCNCNCNNCCTNCCTNCCCNCNTNTCTCTCNCTCNCNANCCNTCTNNCTCTTATNCCNCACAN | | |
| | | TANATCCCACACCTCTNNNNTTCCCNCCTTCNTCNCNCNTCTNCCNNCNNNCCTTNNCTCTTCCNCCTCCNANCTNACTA | | |
| | | CANNCTCNTTCTTTCCNNATCNAACTNCTCATCTCTNNCCCTCCCTCTTTTCNTCCACCTTACCTCNCCCTTCNCTTTCT | | |
| | | CCCTNCTACCACNNCTTNCTCCTNCCNTNCTNCCATNCCTNTCCTNNNANNNNTNNCTCTTCTCCTCTCTNCTCATACNT | | |
| | | CNNTTCACCATNTCNTCTCTTCNCTTACTCACTCACTCTNTTACNTNNTNCNTACTCCTCTCTCTCTNTACTNCCANCTC | | |
| | | TCCNCNCNCTTCCTCATCTCCATCNTCCNTCATCATNTCNCTNNCNACACTTCTNTCTCTNTCTCCTCTANCCTCNNTCT | | |
| | | NCTCTCNCATACTTCCNTTCTCTNCTCNCTAATCCCACNCTNTCTCNNACTCTCNCTCTNTCCNNTTCTCCTNTTCACCT | | |
| | | CTATCCNCNNTTNNNCCCTCTNACTCNNCTCNCTNCNTCCTCACTNCTNNCNTCTCTCNCNTCTNNNTNTCCCTCNNTNC | | |
| | | NTCATCTCCNTCATNACCNNCTTACNCCATNTACCCTCCNTCCTNCTCTCTCANTCTTCCCCCCTCNCACANACTCCNCT | | |
| | | TNNTCCTNCNCCCCCTCNTCTNTNTCTCNNTCANCNATCTCTAAACTACCNCTNCCNATCTCACANACNTCCTCTCTNTC | | |
| | | CNTCTCNGNC | | |
| | Database: | >emb\|X98475.1\|MMVASP M. musculus VASP gene | | |
| | | Length = 2261 | | |
| | | Score = 192 bits (97), Expect = 3e−46 | | |
| | | Identities = 131/145 (90%) | | |
| | | Strand Plus/Plus | | |
| Clone # | T7 | NNCCGNCCCCCTTCNACCGGCTCGGATCCACTAGTAACGGCCGCCAGNGTGCTGGAAAGGGCAAGAGCCTCCACCCCT | | |
| 106 | | TCTACAACCAGCACNATGCGCGAGATCGNGCACATCCAGGCGGGCCAGNGTGGCAACCAGATCGGCGCTAAGTTTTG | | |
| | | GGAGGNGATAAGCGATGAGCATGGNATCGACCCCACTGGCAGTTACCATGGCGACAGNGACTNGCAGCTGGAGAGG | | |
| | | ATCAATGTGTACTACAATGAAGCTGCTGGNAACAAATATGTACCTCGGGCCATCCTAGNGGACCTGGAGCCGGGCAC | | |
| | | CATGGACTCAGNGAGGTCGGGACCATTCGGCCAGATCTTNAGGCCAGACAACTTTGNGTTCGNCCAGAGNGGTGNAG | | |
| | | GAAATAACTGGGCANAAGGCCACTACACAGAGGGNGCCNAGCTGCNGGANTCTNTCCTAGATGNGNAGAGNCAGGAN | | |
| | | CNTTCCCCCNCCCCNNNCCTCCCNCNCCTNCNNCCTNCCCTCTCCCCCCNCNCCTCTNCTTTTCNNTTCTCCTTTTTCTN | | |
| | | TTCCNCNTTNCTNNNNCTTNCTCNCCCNCCTTTNNCCNCTNTNTTCTCTTNCTCCTCCCTTTCCTNTTCTCNCNTCTNNC | | |
| | | NCTTCTCNNNCCTNTCNCTTCCTNTNNCNTNNCNNNNNTCCNNTCCTCNTNNCTCTNNNCCNNNTTNCCTNNCNCTTTCT | | |
| | | CNCCNTCCTCNCCTCNCTCTTCNTNNTTCNNNCCCNNNCNCTNTCNCNTCNCTCTCNCTTTCTCNCNTTTANNCNNTCNT | | |
| | | CTCTCNTTNTNTTCTNCTCNCNCCCTCNNTNCTNCTNCNNCTNNTNCTNTNCNTCTTCTTCTCNCNNCCNTCTNCTNCCT | | |
| | | CTCNTCNNTCTNCTNCNNCTCNTNNNNCTCTCTCCCCTCCTCTCTCTNCNCCTNNNNTCNCNCNNNTCNCCCCTCCTNNN | | |
| | | NCCNCTCCNCTCNNCTCTCTCTCNTNCTCNCNCTNCTCTCTCTNTCTCCTNNTNCTCNTCNCNTNNCTCTCNNCCCTCTC | | |
| | | TNTNNTTNCCNNCNTCNCNNTTCTCTNTCCTCNNNCTCNNCTCNTCTCNCCNTCNNCTCNCNTNCNTCTCCNNNNTNCTC | | |
| | | TTCNCNTCNNCCCNNCTTNNCNCCTNCNTCNTCTNCNNTTCCNNTTCTCNTTCNTCTCNTNNCCCTCCNCNNNNTNNNNC | | |
| | | TCNNNTCCTCTNNNNCTNCNTCTCCTNCNCTCTCNTTTCNTCCNTCTNNCTCCNNNTNNCCTCNCNNCNCNTNCCCCTCC | | |
| | | TTNNNNTCNTCNCNTCTNCTNTTNTCNCCNCTCCNNNCTCCNTCNCTNCTNCTCNCNTCTNTCNTTCCNCNCTTCNTCTC | | |
| | | TTTCTNTCNCCNCCNNCTCCTCTCTCNNCTTCCTNTCCTCNANNCTNCTNTTTNTCNCTCNTCNNTTCTCTNTACNNCTN | | |
| | | TCTCTCCCNCTCCTNCACCNCNNTCTNTCNTNC | | |
| | Database | >emb\|X033691\|RNTUBB15 Rat mRNA for beta-tubulin T beta15 | | |
| | | Length = 1592 | | |
| | | Score = 492 bits (248), Expect = e−136 | | |
| | | Identities = 326/359 (90%) | | |
| | | Strand = Plus/Plus | | |
| Clone # | | ACCGNGNCAGCCCNCTNGGNANGCNTGGAACACTAGCAACGGACGCNAGACGACGCNGGAAAGNANGGACAAGNNAA | | |
| 107 | | GAAGCANCCNNNCCCCCTATCCCCCNCCAGNANNAATNGNAGGANNGGGGNCAGGAGNAGNAAAGGAANAGAAGNGCN | | |
| | | AGGTNCAGNNNAAGCCAGANGNCNGAAGNNACAGAGAGAACAAANCAGAGCNATGGAACAAACNGNGNNCCAAGTGAA | | |
| | | GACNAGNAGCAGNACANNNGNGAGAAGAGGATCNAAAGAANACNGAAGAAAGAAGGCCCAGACNNNTAAANTAAGAAG | | |
| | | ATNACAGGACAGNTGNAGANAANAGAAGGGNGCCNNAGNAGNCAATCGCACAAATCNCCACTAAAGCACGGAAATATG | | |
| | | NCANGAANGCANGAAAACAAGTNGAACAANNANCTNGGTANNANANTNCNNCANAANCTANNNCNNATANCCNAAACCN | | |
| | | NTNNCNNNCNANCNTNNNTACNAACCCNNNCNAANNAANACNTNNGANACNANNTTCAACCAACCATNNNCATCNNANNC | | |
| | | ACNTCCTAACCNAANCNCATNGCNATCGNTCNTACATNNTCTCTACAACNGACCTCNACACCAATCATCNCNNGATACAT | | |
| | | NATCANCNCNTCNATNANCACACTAGTACNNTATATNCATATCTNGNTCACCANNTCAAANATNCGCACACCNNAACNTN | | |
| | | NNTATACTACCACTCANTCTNANGCACNNTACCACATNACAAACCNATAACACNCCTNNCACACNCCATAACATTNNCAN | | |
| | | CNCTACACNNTNNANAGATATNACNTNNNANNNTCNANTCNGCCANNAATNCNCTATNNCACCNTNATCCATCCAANATN | | |
| | | NNNCTCTNNNCGTACANACCCCNNACATCCTNNTNACANNTACNCAATAACANGCNNTANCTCACANNANCNACACATNN | | |
| | | ACNTCNNACTNTNGNGTAACNNATACNGCNNTCANCCNCAAGNCACCCATCNANNNTANCTATCACGCCACANANATCT | | |
| | | GNCCNNTATACCCACNTATANNCTATCACATANNACNTNTAACANNNATATNANACCACACNCCAACNCNCNNATNTATN | | |
| | | NCAACATACANNGGNANNACNTATNTNTCATATATTNNANNANTNANNCANNNACNNTNACACNNCACNCANTACTNCNN | | |
| | | ACNNNNTNANATCGTANATANCCTCGACGNGANTCACTNTNGNTCGACTNCAGACNTATCTNCNTATAATCANCACTATA | | |
| | | CCATNCACACGNCTATNTCTNTNNATCNANTACACACTNCACNCTACNCGCATAACTCTCCCACATCCNAGACNTACATN | | |
| | | TCTCNCAANACAACCTCTANANAATNNTACACTCACACNTNCNTANTCNAGCTNCANTACTANNNCNTNTCACCTACNCN | | |
| | | NNNNTGTACNCTNGANGTATGNACNACANNGANNCANATACTATAATACNCNNCNNATCGNACAAATANNCACAAATCA | | |
| | | ANATNTANACGNCACNCCAAATNCANACNANNNNNNNAATGGACNCAANTCNCANACACTCTACNAANAANNATANACGN | | |
| | | ACTCNCACGANACACGNNNTNAGACATNTANCACNACCNACNNCNANTNCATCNNCTAAGNCATNNNTACAACCCNNCAC | | |
| | | ANNNCNNTCTCAGANNNCGNCATACANTNNATNACNCNNAAGNCCATTANCNNCANNACGACANNGNNA | | |
| | | =>>gb\|BE226644.1\|BE226644 ia23h11.y1 Mouse E10 5 12 5 Pancreas cDNA Library Mus musculus cDNA 5′ similar to SW:NUML_BOVIN Q01321 NADH-UBIQUINONE OXIDQREDUCTASE MLRQ SUBUNIT; | | |
| | | Length = 479 | | |
| | | Score = 38.2 bits (19). Expect = 9.9 | | |
| | | Identities = 29/33 (87%) | | |
| | | Strand = Plus/Plus | | |
| Clone # | T7 | TTTCCCCCCNTCNCCGGCTNGGATCCCTNGTAACGGCCGCCAGTGTGCTGGAAAGCCGNAAGCGCAGAGCAGAGAGG | | |
| 108 | | AGCAGCGGGCTTGTCGNNGGNGGCGGNGGCGGAGGCCGCCATGGATCGCGATGAGGAACCTCTGTCCGCGAGGCCG | | |
| | | GCGCTGGAGACCGAGAGCCTGCGATTCCTGCACGTGACAGNGGGCTCCCTGCTGGCCAGCTATGGCTGGTACATCCT | | |
| | | CTTCAGCTGCATCCTACTCTACATTGTCATCCAGAGGCTCTCCCTTCGACTGAGGGCTNTGAGGCAGAGACAGCTNGA | | |
| | | CCAAGCCGAGACTGNTCTGGAACCTGATGTTGNNGTTAAGCGGCAAGAGGCTTTAGCAGCTGCTCGTTTGAGAATGC | | |
| | | ANGAAGATCTAAATGCCCAAGTNGAAAAACATAAGGAAAAACTAAGACAGCTTNAAGAAGAGAAAAGAAGACAGAAGA | | |
| | | TTGAAATGTGGGACAGCATGCAAGAAGGCAGAAGTTACAAAAGAAATTCAGGAAGGCCTCAGGAAGAAGATGGTCCT | | |
| | | GGACCTTCTACTTCATCTGTCATCCCCAAAGGAAAATCTGACAAAAAGCCTTTGCNAGGAGGNGGNTATNACCCTCTG | | |
| | | ACGGNTNAAGGGNNNTNNACCCTNCTNCTGNNGACCTNNACNCANGGNCCCATCATNTGNCNNCTTNNAACTTNNNACT | | |
| | | CTTTGNNNGNNGNNCNCTCTTNNCNTTANCCNNN | | |
| | Database | >gb\|AF157317.1\|AF157317 Homo sapiens AD-015 protein mRNA, complete cds | | |
| | | Length = 1209 | | |
| | | Score = 327 bits (165), Expect = 4e−87 | | |
| | | Identities = 386/463 (83%) | | |
| | | Strand = Plus/Plus | | |
| Clone # | T7 | CGCCCCCCTTGGTACCGGCTTGGATCCCTNGTAACGGCCGCCAGTGTGCTGGAAAGGCATGACCAAGAAGATGATGG | | |
| 109 | | ACAAGGNGGAGTTTGTCCATATGCTTAATGCTACAATGTGTGCTACCACCCGGACCATCTGCGCCATCCTGGAGAACT | | |
| | | ACCAGGCAGAGAAGGGCATCGCTGNGCCAGAGAAGTTGAGGGAGTTCATGCCGCCAGGGCTCCAAGAGCTGATCCC | | |
| | | GNTTGNGAAGCCTGNACCCATTGACCAGGAGCCATCTAAGAAGCAGNAGAAGCANCATGAAGGCAGNAAAAAGAAAG | | |
| | | CGAAAGAGGTCCCCCTGGAGAACCAGCTGCAGAGCNNGNNGGTNACTGAGGCCTGAGCACTNCCAGACTTACTTACT | | |
| | | AAGNCTGCTGNGNTCCCAGGCCTTGCCCTGNCACAGNGAGGCCGCGNANNACTCCTCNNCCTGCCNCCTCTNCNTNCC | | |
| | | AGCTCCTGCCGANNCCNNCTGCGCCGACNGNGTACNNNCCCCTTCTCCTNCNACNCNNCNCCTNCNCTNCTTTCCCNTC | | |
| | | TCCNCCCNCNCCCNCNNCCCCCCTTCCCTTCCNCNCCCTTTCCCNNNCNTNCCTTCNTCCCNCNNCNCCNCTCCTCTCC | | |
| | | CTTTNNTNNNTCCTNCNCTCNCTCCNTNNCTCNCTCCNNCNNNCCCCTCCTCNNCCTCTCNCTTCCTNTNCNTCCTNNNN | | |
| | | NNCTNCNNCTCTCNTNCCNNCNNCCNCTNCTCTCCTTCCTCNTCTCCTCTCCNTCNCNNNNTCTTTCTTCNNCTCTNCTN | | |
| | | CTNNNNCNNNCNNCNNCNTCCNCNCNCTNCNCNCTCCNNNCNNNCTTNCNTCTNCNCCNNNCNTTCTCCTCCTCCTCCTC | | |
| | | CNNCNCCNCNTNTCCNCCNCCCNNNNCCNCTCCTCNNNCCCNCTNNNNNNTNTTNTCTCTCNCCTCCTCTCNNCTNTCCC | | |
| | | CCCNNCCTTNCCCCTCCNTCCTNNTNCTCCCCNTCCTNCCTCTCNNNTNCTTCNNCTCCTNTTCTCACNTTTNCTCNNCN | | |
| | | NNCNCTTCCTCCNCCCTTTCCCCCTTCNCTCCCCNCCNNTTTCTCCTCTCTNCCTCACTCCTCTTCTCTTNCTCCTNCTT | | |
| | | CTCCNCCCTCTCCTCCCTNCCTCCCNNNTTNCTTTCCNCCCTCTCNTCTCTCCCTCCCTCCCNCNTTCTCNNTNCTNNTC | | |
| | | TTTCNCTCTCTTCTTCNTCCCCTCCNNTCCC | | |
| | Database | >gb\|M88136.1\|CRUSTSTA Cricetulus griseus seryl tRNA synthetase mRNA, partial cds | | |
| | | Length = 598 | | |
| | | Score = 375 bits (189), Expect = e−101 | | |
| | | Identities = 257/281 (91%), Gaps = 1/281 (0%) | | |
| | | Strand = Plus/Plus | | |
| Clone # | T7 | CCNNACTNCACAACCTCTTTCNACCGCGCCTATNCNCANCCACCTANNNTANACTGGGACACGNACAGATGNCGCATGN | | |
| 110 | | GAAAAGCAACTGACATACNNCCGANNNACTAANACCTTCTAGTNCANCCAATTTTTNTNCCTNNTNAACAANAACACTCA | | |
| | | CTNTTTNCCNANTNCTACTNNNTGTANTCNANATNTNNNANCTTCTNNGNTCTANTANCTTTTNNCTCCANNCCTNCTCC | | |
| | | TATNNANCATATCCCNATCTNCTCCACTCNNTNTANNTACCCACTCCTCCTCNCCCANCNCTTTCCCNCTATCCANACTC | | |
| | | NNTCTACTCNTCNCTCCCTTCNNCNCANTTCNCCATNTTNTCTCCTNATCNNTTTNNNTCCCTCNTNCCNNNTNNACNCA | | |
| | | NCCTTCTCCCCTTNTTNNCNNNCTTNCNTCTTNNCNCCATCCCCTNTCCCNCTCNCTTCANTNTNTATCCCCCTNTNNCC | | |
| | | CNCCCNTANNATTCNTCNACCTAACTACTCNTCTCTTNTTTCNTCCANNNNCNCCNCCCCCTNCTCTNACTCNTCCNTCN | | |
| | | CCTTATNATNTCTCCATNCACTNNANNNTCCCACTNNACNCTCATTNNTATATCCNTCNNCTCTTCCACCNCNCTNCTNC | | |
| | | TACANNNNCTCNCCTACTCTCNCCTTTACNATNCTACCTNNNTANACTNNCNTCNNANNTNTNTATTCTNCAANACTNTC | | |
| | | TAACCCCCTANTCTGTCNCNTTNNCNCCTNCTNNNNNTANTCTCNTNATNTTTCTANNCCTTCCTNTCNATNANTTCTCA | | |
| | | TCTACNTCNNNNTNCNTCNTTTTNNTCATTCCTCNTNAATCTCCTNATNNTAANTTATCNTCCCAANCNNNTNATCAATN | | |
| | | TACTNNTCTNANCACTCTCTNCCTCCTNTCCNCCACNCNANTCTACTNAACNNTNCCTCNCACTNTNNTNTTCANTTNCA | | |
| | | NANATNACNTNNTNCCNTATCTTCANTCTCCNACTCTCTACNCACTCTTNTCANATCCACTTCATCCTCNNCNTCTTNAT | | |
| | | CTCNCNNNTTATTANCCNTACCTCTNTCNTCNTATNNTNCCTTCANTTCCCTACCCTTCACTCTTCTACNNCTCCNCNTC | | |
| | | TCCTCNCANCCTCACCTCTCTCCTCTCNCACTNNNNCTCTNNATACNGCNTANTCATCNCTCCACNTCTACNTANTNTNT | | |
| | | NTATACACTNNACNNCTTCANTCACTNNTTACATCATTANNNTCTCTCCNCTATNCCCACTTANTCCCTCACTATACTAT | | |
| | | CTATCTCTCANCCTNATTTNCTCCTCCNNCTATCTNCNTANNCTCTCACTTTTCTCATNATCTCCANCNTNTCTNNACNC | | |
| | | ANATCTCNTCNATCTNATNACTCTCTCCTNTCCATCNCTCNTCTNTCTNCATCTANCNTATTCACNCCGACTNCTNCCTC | | |
| | | ANTNCACANATTANNNN | | |
| | | No homologous sequences in the databases | | |
| Clone # | T7 | AGGCAATGGCGGACGTGTCTGAGAGGACGCTGCAGGTGTCCGTGCTAGTGGCTTTCGCCTCTGGAGTGGTCCTGGG | | |
| 111 | | CTGGCAAGCGAATCGGCTGCGGAGGCGTTACCTAGACTGGAGGAAGCGGAGGCTGCAGGACAAGCTGGCAACGACT | | |
| | | CAGAAAAAGCTGGACCTGGCCTGAGCACGCGCTGCAGCCCGAGTCCGCCGGGTTCTCACTCCCTAAGCCCAACGCAG | | |
| | | CCCGGATCGTGGGAGCCGCGCGACCCAGGAGTCGTCCTTGCACGGCTTGCAAGAACATGGCTTGCTTCAGAAAGAAA | | |
| | | ATAGTTTTGTCTTCTCTAACAACTTACTTTCAGCTTGTCGAAGATGAAAATAAAAAGCACTGGAGAGAAATAATTTCTT | | |
| | | GCACTTTATGAATCTATTTTTAAAATAAAAAAATTAAACATC | | |
| | | >ug\|AW106096.27359\|Mm.27359 um23a10.y1 Mus musculus cDNA, 5′ end | | |
| | | Length = 539 | | |
| | | Score = 803 bits (405), Expect = 0.0 | | |
| | | Identities = 408/409 (99%) | | |
| | | Strand = Plus/Plus | | |
| Clone # | T7 | TAGTAACGGCCGCCAGTGTGCTGGAAAGGGACTTTGTTCAGATCTTTTTCTGATCGTGCATCAGCTGCTGGTTCATT | | |
| 112 | | ATAGCCTTCTCTTAAGCACTGAATAAGACAAAAAATGAATCTTGGAAGAACAAATTCAGACATCATCAGTAAGTCTTTG | | |
| | | GGGACACAGGGAATATTTGAACTTGATTTAATCTGATGTCTTCTACAAAACCCGCTCTCCCGCATTACGTTGCTGTCC | | |
| | | CCGCAGTCGCAGGCGCCCCCGGCCTGGCTGCGGAACATGTTGAAGTCGTGTCCAGTGTGGTCGCCCTGGTGGAAGC | | |
| | | ACTCCGCGCACAGCGACATGCAGGGGGAGATGCCGCACGTCCGGCAGCGGTAGGCCACGAAGTTGGCCGTCCAGAC | | |
| | | CAGGCCGCAGAGCGCCGCGGGGTCGTAGGCCCGCACGGCCGCGCAGAACTCCTCGTAGCCGCCGCCGCCGGCCAGA | | |
| | | AGGCACTTACACCACTCCAGGGCATCCTCCTCCGCCTCGCCCGGGCCGCCGCCGCCGCCGCCCGCCGCCTCCTCGCCG | | |
| | | CCCGCAGCCCCGGCCAGC | | |
| | | =>>gb\|AI413025.1\|AI413025 EST241326 Normalized rat brain, Bento Soares Rattus sp. cDNA clone RBRDZ28 3′ end. | | |
| | | Length = 544 | | |
| | | Score = 619 bits (413), Expect = 0.0 | | |
| | | Identities = 451/463 (97%), Gaps = 3/463 (0%) | | |
| | | Strand = Plus/Minus | | |
| Clone # | T7 | AGGCGACGCGCGCATGGAGGCCGGCTGAGGAGCGCCGCCGCCTCTCTCGGTAAGGACTGNGTCTGTGTCCCCAGGC | | |
| 113 | | ATCCTACATCAATCAGGAAGCTGCTGTCCAGCCATGGAGGGAGAGGAGAAGCCAGCTCAAGAGGCTGACGTGGAACC | | |
| | | TGTGGTAACAGCAGGCACCTCAGAAGCAGTGCCAAGGGTGCTTGCTGGAGACCCTCAGAACATCTCTGATGTGGATG | | |
| | | CCTTCAACTTGCTCCTGGAGATGAAACTGAAACGACGGCGTGAACGCCCCAACCTTCCACGTACAGNGACCCAGCTAG | | |
| | | NGGCCGAGGATGGGAGCAGGGTGTATGTGGNGGGCACTGCTCACTTCAGTGATGATAGCAAGAGAGATGTAGTAAA | | |
| | | GACTATCCGGGAGGTGCAACCGGATGTGGTCGTGGNGGAGCTCTGTCAGTACCGGGGTGTCCATGCTCAAGATGGA | | |
| | | CGAGAGGACGCTGCTGCGAGAGGCCATGGAGGTCAGCCTGGAGAAGCTGCAGCAGGCTGTCAGGCAGAATGGNTTA | | |
| | | TGTCTGGACTCATGAGATGTTGCTGCNTGNAGGTGTCTGCTCACATCACTGAGCAGCTGGGCATGGCCCCTGGTGNC | | |
| | | GAGTTCAGGGAGGCCTTCAAAGAGGCCAGCAAGGTACCATTCTGCAAATTCCACCTGGGNGACCGACCAATCCCAGN | | |
| | | CNCCTTTAAGAGGNCCATTNCTGCACTCTCCTTCTGGCANAAAGTCAAGCTTGNNCTGGNNCCTGNGCTTNCTTGNCN | | |
| | | GACCCAATCANNNNNNGNCNACNNNNNNNNNCTTNAANT | | |
| | Database | >emb\|AL02232B.21\|HS402G11 Human DNA sequence from clone RP3-402G11 on chromosome 22q13.31-13.33. Contains the MAPK12 gene for mitogen activated protein kinase 12 (SAPK3), the MAPK11 gene for mitogen activated protein kinase 11 (PRKM11), gene KIAA0315, the gene for a novel protel> | | |
| | | Length = 177241 | | |
| | | Score = 121 bits (61), Expect = 4e−25 | | |
| | | Identities = 124/144 (86%), Gaps = 1/144 (0%) | | |
| | | Strand = Plus/Plus | | |
| Clone # | T7 | CTTGTAACGGCCGCCAGTGTGCTGGAAAGTGACCGGCTACCAGTGCAGAGCATCCCAGGCCCCAACCTTTCCTGGAC | | |
| 114 | | AGTTGGACCTCTTCCTGAGAGACCCCGGAACGTTTTCTACCTTACCCTTGGGAGAAGAGCCTCTGGCTGAATGGCAG | | |
| | | CTGTGGATGACTTACAGTTTGAAGAATTTGGCGATGGAGCTACACTGTTAGCAGCAAATCCAGATGCTACCACGATAA | | |
| | | ACATTGAGGACCCCAGTGTATCTTTTGGCCACCAACCCAGACCCCCCGGGAGCGTGGGGCGAGAAGAGGATGAGGAG | | |
| | | CTGCTGGGGAACAACGACTCTGACGAGACCGAGTTACTTGCCGGTCAGAAGCGAAGCTCTCCCTTCTGGACATTCGA | | |
| | | ATACTATCAGACATTCTTTGATGTGGACACTTACCAGGTCTTTGACAGAATAAAAGGGTCCCTGCTGCCGGTTCCTGG | | |
| | | AAAGAACTTTGTGAGGCTGTACATCCGCAGCAATCCAGATTTCTATGGTCCCTTTTGGATATGTGCCACGTTGGTCTT | | |
| | | TGCCATAGCAATTAGTGGGAACCTTTCTAACTTCCTAATCCATCTGGGAGAGAAGACATAGCATTATGTGCCCGAATT | | |
| | | CCAGAAAGTGTCTATCGCAGCGACTGTCATCTATGCCTATGCCTGGCTGGTC | | |
| | Database | >dbj\|AK000823.1\|AK000823 Homo sapiens cDNA FLJ20816 fis, clone ADSE00693 | | |
| | | Length = 1538 | | |
| | | Score = 363 bits (183), Expect = 6e−98 | | |
| | | Identities = 306/347 (88%) | | |
| | | Strand = Plus/Plus | | |
| Clone # | T7 | CCCANACCTCTNTCGNCACGAGCTCGGATCCCTAGTAACGGCCGCCAGTGTGCTGGAAAGCCCGTCCNNAGCGCTCCG | | |
| 115 | | GCGAGCTGGCCTTNGCTGCAGTNGAGCCGGGCTNGNACTATGNAGNGCCTGGCCATGGACCTGCGGGTGCTGNCCC | | |
| | | GGGAGCTGGCGCTCTACCTGGAGCACCAGGTTCGCGTCGGCTTCTTCGGCTCGGGCGNGGGCTTGTCACTGATCTT | | |
| | | GGGATTCAGCGTTGCCTATGCTTGCTACTACCTGAGTAGCATTGCCAAGAAACCCCAGTTAGNGATNGGAGGGGAGA | | |
| | | GTTTCAGCNCGCTTCCTACAGGACCACTGTCCCGNGGTGACAGAAACCTATTACCCGACGGNCTGGNGCTGGGAGAG | | |
| | | CCGAGGACAGACATTGCTGAGACCCTTNATNACTTNCAAACCCCCGGTGCAATACAGGAACGAACTCATTAAAACTGC | | |
| | | AGACGGAGGACAGATCTCACTGGACTGGTTTGATAACAATAACAGTGCGTACTATGNGGATGCCAGCACCAGACCNT | | |
| | | ACTATCTTGCTCTTGCCTGGCCTNACTGNAACAAGCAAGGAATCCTACATCCTTCATATGATCCATCTCAGTGAAGAAT | | |
| | | TNGGATACAGGTGNGNGGNTTTTAATAACANAGGAGTAGCANGAGAAAGTCTCTTGACACCACCGGACTTACTGCTG | | |
| | | NGCGAACACTGAAGAACTTGGAGGCCGNNGTCCACCACGTGCACAGCCTGTACCCTGGGGCNTCCTTTNCTGGCANC | | |
| | | NGGCGTATCAATGGGGAGGGAAATGCTGCTTTTNGNANTTACTTNGGGTTAAAAATTGGGTCCAAAAANCCCCNTCTT | | |
| | | GANGGCNTNCCTGCNACCNNTTTCCC | | |
| | Database | >gb\|AF007152.1\|AF007152 Homo sapiens clone 23649 and 23755 unknown mRNA partial cds | | |
| | | Length = 2044 | | |
| | | Score = 456 bits (230), Expect = e−126 | | |
| | | Identities = 5311634 (83%), Gaps = 4/634 (0%) | | |
| | | Sirand = Plus/Plus | | |
| Clone # | T7 | TTCGACCCCCTTGGTACCGAGCTCGGATCCCTAGTAACGGCCGCCAGTGTGCTGGAAAGGGCGCAGCCGGCCTAGCG | | |
| 116 | | AGGTCAACATGCCGGTCGCCAGAAGCTGGGTTTGTCGCAAAACCTATGTGACCCCACGGAGACGCTTCGAGAAGTCG | | |
| | | CGTCTCGACCAGGAGCTAAAGTTGATTGGAGAGTATGGACTCCGGAACAAACGTGAGGTTTGGAGGGTCAAGTTTAC | | |
| | | CCTGGCCAAGATCCGTAAGGCGGCCCGGGAGCTGTTGACGCTGGACGAGAAGGATCCCCGGCGTCTGTTTGAAGGC | | |
| | | AATGCTCTCCTGCGGCGGCTTGTTCGCATTGGGGTGCTGGACGAGGGCAAGATGAAGCTGGATTACATCCTGGGAC | | |
| | | TGAAGATTGAGGATTTCTTGGAGAGGCGGCTGCAGACCCAGGTCTTTAAGCTGGGCCTGGCCAAATCTATTCACCAT | | |
| | | GCCCGTGTGCTCATCCGCCAACGTCACATTAGGGTCCGCAAGCAGGTGGTGAACATCCCATCCTTTATTGTTCGCCTG | | |
| | | GACTCGCAGAAGCACATCGACTTCTCCCTCCGTTCTCCTTATGGNGGCGGCCGTCCAGGCCGAGTGAAGAGGAAGAA | | |
| | | TGCCAAGAAAGGCCAGGGCGGGGCTGGAGCTGGTGATGATGAGGAAGAGGATTAATTCTTGGCTGAACTGGAGGAT | | |
| | | TGTCTAGTTTTCCAGCTGAAAAATAAAAAAGAATTGATACTTGGAAAAAAAAAAAAAAAAAAAAGGGCNGCCNGCTCG | | |
| | | AGCATGCATCTAGAGGGCCCTATTCTATAGGGGCACCTAAATGCTAGAGCTTGCTGACAAGCCTTGACTGNGNCTTCT | | |
| | | AGTGNCNGNCN | | |
| | Database | >emb\|X66370.1\|RNRPS9 R.norvegicus mRNA for ribosomal protein S9 | | |
| | | Length = 688 | | |
| | | Score = 932 bits (470), Expect = 0.0 | | |
| | | Identities = 577/613 (94%) | | |
| | | Strand = Plus/Plus | | |
| Clone # | T7 | TNNGNCCCCCTTNTACCGGCTCGGATCCACTAGTAACGGCCGCCAGTGTGCTGGAAAGCGGCGGCTCTGAGCGCCGG | | |
| 117 | | AGCCGCCCCGGGACATCATGGCGGAGGTCTGCGAGCCCACCCGCCCCTCGGAGGACGAGGACGAGGAGCGGGAGCC | | |
| | | GCTGCTGCCTCGCGTTGCCTGGGCCCAGCCGCGGAGGGTCGCGCCCGGGAGCGCCGNGAGAATGCAGGCGGACGAG | | |
| | | GGCGCGGATGTCCTCCGCGAGCCCGCTACCGACGAGCCGCCGGCGGTGTCCGGNGAAGGGTCGANCTNNGCGAGCT | | |
| | | TGTCCNNNGAGCTGGATNTNGACCCNCNCTACNCTCTCANTTTACNNTNATATTNTTTNNCGATTCACNATTNTTCTCC | | |
| | | TTCNTGNCTNTNCNNCCNNTCNCCNCCNTNCNCTCTNTCTGNCNTCCTNNNTCCCTNTNNTCCTCCCTCTNTTNTNTNCC | | |
| | | CTCANCCCCCNNNNNCCNCNCTTCCCNCNCNTCTCCCTTCTCTCNCCTCCNCCCCNCNTNNTNNCTCCTCNNCNNCCNNT | | |
| | | CNCNNNNTCNNTNCTNCTNCCCTNCTCCCNNNCTCNNNNNNCCCNCCCTCTTCNTNTCNCTCCNNNCCNCNTCTCNNCCT | | |
| | | NCCCCCCTNCCNTCTCNCNNNTNNNCCNCNNTCNCCCTCTCTCCCNCCTTTCTNTGTTCTCNCNNTCCTNCTNCCNANTN | | |
| | | TCTNCCCCTNACCTCNNCCNTCTCNNTNNCNNCTTTTCNNCCTCTCCCNNNNNTCCTTTCTCANCCCTTGTNTCCTNNNC | | |
| | | CTCTNNNCNCTNNNTCTCCNCTCCCTCCCNNCCNCTCTTCNCCTNNCCTCTCTCCTNTCTNTTNTCTCCNTNTCNTCCTC | | |
| | | NNCNCCCTNTTCCCNCATCTCTTCCCTTTCTNTCCTCTCCNTTNCCTNCCCACGTCTTCCNTNNANCCCTNNNANNNNNT | | |
| | | CCTTCTCTTNCNTCNTCCCCCCTCNCNNNCNCTTCTNNTNCTCNNCCNNNNTACNTNTCTCCCCNCNNTCCCTTTCTTCT | | |
| | | NCNNCTNNNNCTCNNTTTCNCCTCNTCNNNCTCCTTTCATNCTCTCCTCCNNCATNTCCTCNNCTNCTCTCTCTCTATCN | | |
| | | CCTCNCTNTCCATCNCNCTCTNTCTTCNCNTNCTNCCTCCTCCNCNCCTCNNCTCTCNNNNNCCCTCTNTNNNNCTCNTC | | |
| | | CCTCATCTNCTCTCTCTCCTTCTNNNCNCNTCGTCNCTNTCCNTTCNCTNNNTNC | | |
| | | >gb\|BE161116.1\|BE161116 PM3-HT0424-170200-001-b11 HT0424 Homo sapiens cDNA. | | |
| | | Length = 132 | | |
| | | Score = 75.8 bits (38), Expect = 3e−11 | | |
| | | identities = 38/38 (100%) | | |
| | | Strand = Plus/Minus | | |
| Clone # | T7 | CCCCCTTCTTGGTACCGAGCTCGGATCCCTAGTAACGGCCGCCAGTGTGCTGGAAAGGGAGCAGCAAACGGCCGGCG | | |
| 118 | | GCAGGCGCCGCGCGGGGGGCGGGCGGCGCGGAGGCGGCGGTGGCCATGGCCGAGGCGTCGCCGCAGCCCGGACG | | |
| | | GTACTTCTGCCACTGCTGCTCGGTAGAGATCGTGCCGCGCCTGCCGGATTACATCTGCCCAAGGTGCGAGTCTGGCT | | |
| | | TCATTGAGGAGCTTCCAGAAGAGACCAGGAACACAGAGAATGGCTCAGCCCCCTCCACAGCCCCCACCGACCAGAACC | | |
| | | GGCAGCCATTTGAGAATGTGGACCAGCACCTGTTCACGCTGCCACAGGGATACAGCCAGTTTGCCTTTGGCATCTTC | | |
| | | GACGATAGCTTTGAGATTCCCACGTTCCCTCCTGGAGCCCAGGCCGATGATGGCAGGGACCCTGAGAGCCGACGGGA | | |
| | | GAGAGAGCACCAGTCTCGGCATCGGTACGGGGCCCGGCAGCCCCGTGCCCGCCTCACTGCCCGCCGGGCCACTGGCC | | |
| | | GGCATGAAGGTGTCCCTACGCTGGAAGGGATCATCCAGCAGCTCGTGAATGGCATCATCTCTCCGGCTGCTGTGCCC | | |
| | | AGCCTGGGCCTTGGTCCCTGGGGCGTCCTGCACTCGAACCCAATGGACTACGCCTGGGGGGCCAACGGCCTGGACAC | | |
| | | CATCATCACGCCAGCTCCTTAATCNGTTTGAGAACACCCGGNCCCCCCACTTGCAGACAANGAGAAGAATTCAGGCTN | | |
| | | TTCCCCNCGGTCCCAGTCACAGANGAACACNGTGGGCTTCANGGGNTTGAGTGCCCAATGGTGTNAAGNAAGACTATT | | |
| | | CCNCTGGGTNANAAATNTGCCGGCAAANTGCCCTTGNAACCNCTTGTTCCCNGANAAGCTGCATCGNNCCCNTGCTTN | | |
| | | GANCNANCATNACAGCTTGNCCGGGCTGNCNTAAGANCCCTCNNTGGACAGAAACNCAGCCCCCAATCCCCCAGCTTT | | |
| | | GACCGGGGAAGGTTNTTCCTCGCCNNGNCTTAANCCTTCTCCANTTCNCCACNATTGGAACCCCCCAAGNAAAATNTG | | |
| | | GNGCCNNCTTGCNAACCCCCCGGGGNTGNGANTCCCCNCCCCGGGGCCCAAAACGCCNCTTTCCCCCCCCNGCTTGGG | | |
| | | NAAAAAGGTTTCCCTGGGGCCGAAACNTGGGGGCCCANTTTGNNNNNTTTNATTCANGGCCNCTTTTGGGGNTCCNG | | |
| | | GNCNNTCCNNGGNNGNTTTTTTTTN | | |
| | Database | >dbj\|AK000559.1\|AK000559 Homo sapiens cDNA FLJ20552 fis, clone KAT11732 | | |
| | | Length = 1362 | | |
| | | Score = 547 bits (276), Expect = e−153 | | |
| | | Identities = 527/610 (86%), Gaps = 7/610 (1%) | | |
| | | Strand = Plus/Plus | | |
| Clone # | T7 | ATGGCGGCTCTGCCCTTCCTCATCATCGAGACAAGCACCATTAAGCCTTACCGTCGAGGGTTTTACTGCAATGACGAG | | |
| 119 | | AGCATCAAGTATCCCCTGAAAGTCAGTGAGACTATAAACGATGCTGNGCTCTGNGCGGNGGGGATCGTCATCGCCAT | | |
| | | CCTGGCGATCATTACAGGGGAATTCTACCGGATCTATTACCTCAAGGAGAAGTCCCGCTGCACCACTCAGAACCCGTA | | |
| | | TGTGGCAGCNCNCNATAAGCAAGTGGNATGCNTCCTTTNNGNTGTGCCAATTAGCAAGTCCTTCANNGNGATCGCCAA | | |
| | | AGTGTCCATCGGGCGCCTAAGGCCTCACTTCCTNAGCGTCTGTGAGCCTGATTTCAGTCAGATCAATTGCTCCGAGG | | |
| | | GCTACATTCAGANCTACAGGTGCAGAGGAGAAGNCAGCANAGTACAGGAGGCCAGGAAGTCCTTCTTCTCGGGCCAC | | |
| | | GCCTCCTTCTCCATGTTCACTATGCTGTATCTGGTGCTCTACCTTCAGGCCCGCTTCACCTGGCGCGGGGCCCGACTG | | |
| | | CTCCGCCCCCTCCTGCAGTTCACTTTGCTCATGATGGCCTTCTACACGGGATTGTCACGGGTATCTGACTACAAGCAT | | |
| | | CATCCTAGCGATGTCCTGGCAGGATTTGCCCAAGGAGCTCTGGTGGCCTGCTGCATAGTGTTCTTCGTGTCCGACCT | | |
| | | CTTCAAGACTAAGACGAGCCTCTCNCTGCCCGCCCCTGCGATCAGGAGGGAGATCGTGTCTCCCGTGGACATCATCGA | | |
| | | CAGGAACAATCACCATAACATGGTGTAG | | |
| | Database | >ref\|NM_003713.1\| Homo sapiens phosphatidic acid phosphatase type 2b (PPAP2B) mRNA, and translated products | | |
| | | Length = 1362 | | |
| | | Score = 535 bits (270), Expect = e−149 | | |
| | | Identities = 470/537 (87%). Gaps = 4/537 (0%) | | |
| | | Strand = Plus/Plus | | |
| Clone # | T7 sequence | GGGAAGCTGAAGCAGTTCGATGCCTACCCTAAGACTCTGGAGGACTTCCGGGTCAAGACCTGCGGGGGTGCCACG | | |
| 120 | | GTGACCATCGTCAGTGGCCTTCTCATGCTCCTGCTTTTCCTATCGGAGTTGCAGTATTATCTCACTACGGAGGTGCA | | |
| | | TCCTGAGCTCTACGTGGACAAGTCTCGGGGGGATAAACTGAAGATCAACATCGATGTTCTTTTCCCGCACATGCCTT | | |
| | | GNGCCTACTTGAGCATCGATGCCATGGACGTGGCCGGGGAGCAGCAGCTGGATGTGGAACACAACCTGTTCAAGAA | | |
| | | ACGACTAGACAAGGATGGCGTCCCCGTGAGCTCAGAGGCTGAACGGCACGAGCTTGGGAAAGTCGAGGTGACAGT | | |
| | | GTTTGACCCCAACTCCTTGGACCCCAATCGCTGTGAGAGCTGCTACGGCGCTGAGTCAGAAGACATCAAGTGCTGT | | |
| | | AACAGTTGTGAAGATGTGCGGGAGGCCTATCGCCGTCGAGGCTGGGCCTTCAAGAAGCCCGGACACCATTGAGCAGT | | |
| | | GTCGGCGAG | | |
| | BGH | CCTATCACAATTCATATTTATTGCCCTGGGCTGGGCTGGCTGAGGAGAGGATATGGGTAGTTGACAGGCTGGAGG | | |
| | sequence | GTAAACCCACAGGAGAAGAGAGGGCAGGACAAGCTGTGGGGAAGGGAGAGAGCTACGTTGTCTTCCCTAGGTCAA | | |
| | | TTTTCTTCTGGATGGCCCGAGCGGAGTGGTAGATGAGCGAGTCAATGAGGCCGGCCANTGTAAACATGCCCCCAAT | | |
| | | GATGGCGCACACACCTGTCAGGAAGTGGGTGAAAGACCTGTGTTTCTCCGTCAGCTTCACCATCATTGGTGAGAGC | | |
| | | TCATACAGCACAAAGACCCCCGGNAGGCCCTGGTCACCCAGGAGCCCATTGGCAACCTTCTCGNGCCTGGTCACGGA | | |
| | | NAACTGGTTAGTCCTCAGCACCTCCCCGTCCACCTTCATGTACACTGNGGGCACCACNTTCACAAAGTACTGGAACA | | |
| | | TCATGGAGGCTTGGGGTGCAGTCACGTTGGTGTGGNCTAGGGGGTTCACGATTCCTGGATAGTCCTCCCCGAATG | | |
| | | ACAGGTGCTTGATGTAGTGTGNCATGTTGATATTGTCAAGGCCAAAGCTNTGCAAGTCATGAACATGCACATGAGA | | |
| | | CTGTTGGAAGCTCTTNCCAGGGGCAAAGTGGAAGTTTCCAGCCACCTTGTTGACCTCCAAGAAGCCNNACACCTGGC | | |
| | | AGCCTTCATTCTTCTGCTCCTGCATCTTCTGGCTGAAGCCCTTTCGCGACACTGTTN | | |
| | Total | | | |
| | cDNA, | Human calcineurin B mRNA, complete cds | | |
| | EST cluster | Length = 2548 | | |
| | | Score = 363 bits (183), Expect = 6e−98 | | |
| | | Identities = 291/329 (88%) | | |
| | | gb\|M30773.1\|HUMCNR | | |
| | Motifs | | | |
| Clone # | T7 sequence | CTGGAAAGGGGCGTTGAGCAGCTGGGACCGGAGTTGTGCTCACCGGGGTCGGGCCAGGTCGCTGCTGCTCTGGCC | | |
| 121 | | ATGGCCGAGGCACGCGCATCTCGCTGGTACTTTGGGGGGCTGGCTTCCTGCGGAGCCGCTTGCTGCACGCACCCG | | |
| | | CTGGACCTGCTCAAGGTGCATCTACAGACCCAACAGGAGGTGAAGCTGCGCATGACTGGAATGGCACTGCAGGTGG | | |
| | | TGCGAACTGATGGCTTCCTGGCGCTCTACAACGGCCTGAGTGCCTCGCTGTGCAGGCAGATGACCTACTCTCTGAC | | |
| | | TCGGTTCGCAATCTACGAGACCATGCGGGACTACATGACCAAGGACTCCCAGGGGCCTCTCCCCTTCTACAACAAGG | | |
| | | TGTTGCTGGGCGGCATCAGTGGTTTAACTGGAGGCTTCGTGGGGACCCCAGCAGATTTGGTCAATGTCAGGATGC | | |
| | | AGAACGACATGAAGCTGCCCCCGAGCCAACGACGCAACTACTCTCATGCCCTGGATGGTCTGTACCGTGTAGCCCGT | | |
| | | GAAGAAGGCCTGAGGAAGCTCTTCTCTGGAGCAACTATGGCGTCCAGCCGTGGGGCCCTCGTCACTGTGGGCCAGC | | |
| | | TGTCCTGCTATGACCAGGCCAAGCAACTGGTCCTCAGCACTGGGTACCTGAGTGACAACATATTCACCCACTTTGTC | | |
| | | TCCAGTTTCATTGCCGGCGGATGTGCCACATTTCTGTGCCAGCCCCTCGATGTGCTGAAGACTCGCCTGATGAACTC | | |
| | | CAAGGGCGAGTACCAC | | |
| | BGH | TGCAGGGAAGACAGAAAGGCCTCCCAGGCCACTTGGTTTATTAGATCCTGAAGAGAGGTGTAGGCAGTGCCCCTGG | | |
| | sequence | GCCGCTGCCACCTCCTGGGGGAGGACCTGTGGGAGGCACAGGGCCGAACCTCGTTTTGATACACAGAACCCCATTT | | |
| | | GAGGGAAAACAGGCTGCTTCGAAGCCTGAGGGATGGTGAGGGGTGATGCCTGCCATACAGGAAGCCAGGTCCTGG | | |
| | | GAGGGCACAAACGATGAATCCATCACTGCCCCAGCTCTGCCAGCATGCCCACCTGGCCCTGGGGAAGCCAGGCAAG | | |
| | | GGAGGGCACAGGCGTGTGAGGGACACAGACAGTTCCTGGTGACGGCAGTAGCTGCTGAGCAGGAGGGTTGAGCAA | | |
| | | AACTGACCATTAGAGCAGCCAAGGCTGCATATAGGAGGTGCGCTCGGGAACCCCAGGCACTTTCTCTGGACTCCAC | | |
| | | GGTCATGGCTTCTGCTGGTGATCTGCACTGCCTGCCTGTCCCCTCTCCTGAAGGCACTACCTTCCAGAACACAGCAC | | |
| | | GGTGGTCCCTCTTGTGACAAAGTGCTTGTGTGTGTGCTCTAGTCATTTGGTAAGCAGAAGCTGCGAGGGGCCATAC | | |
| | | CCTGCCACACTACCCAAGTTCTGGGCNGGAAACTGCTCCCTGCACAGAGGGCCAGCGGGAGCAGGAAACGAACTCA | | |
| | | ACTTCGCTGGGCTTGCCAGGCANGGCACGGTAGCAGCCAGGGAGGTTGGGACAGTGACANCAGNCAGGCAGACTT | | |
| | | TTTTGNGGAATAATNGNAC | | |
| | Total | | | |
| | cDNA, | Mus musculus solute carrier family 25 (mitochondrial carrier;adenine nucleotide translocator), member 10 | | |
| | EST cluster | (Slc25a10),mRNA | | |
| | | Length = 2021 | | |
| | | Score = 1404 bits (708), Expect = 0.0 | | |
| | | Identities = 753/768 (98%) | | |
| | | ref\|NM_013770.1\|gb\|AF188712.1\|AF188712 Mus musculus mitochondrial dicarboxylate carrier mRNA, complete cds; nuclear gene for mitochondrial product | | |
| | | Length = 2021 | | |
| | | Score = 1404 bits (708), Expect = 0.0 | | |
| | | Identities = 753/768 (98%) | | |
| Clone # | T7 sequence | ATGCACCACCAGTCTGTTCTGCACAGCGGCTACTTTCACCCACTGCTTCGGAGCTGGCAGACTGCTGCCTCCACCGT | | |
| 122 | | CAGTGCCTCCAACCTCATCTATCCCATCTTTGTCACGGATGTTCCTGATGATGTCCAGCCTATCGCCAGCCTCCCAG | | |
| | | GAGTGGCCAGGTATGGCGTAAACCAGCTAGAAGAGATGCTGAGACCTCTGGTGGAAGCTGGCCTGCGCTGTGTCC | | |
| | | TGATCTTTGGCGTCCCCAGCAGAGTTCCCAAGGATGAACAGGGCT | | |
| | | ref\|NM_008525.1\|Mus musculus delta-aminolevulinate dehydratase (Lv), mRNA | | |
| | | Length = 1080 | | |
| | | Score = 543 bits (274). Expect = e−152 | | |
| | | Identities = 274/274 (100%) | | |
| | | Strand = Plus/Plus | | |
| Clone # | T7 sequence | TTCGCCCACGCCTTGGTACCGGCTCGGATCCCTTGTAACGGCCGCCAGTGTGCTGGAAAGCAACCGCAACCTGACG | | |
| 123 | | TTGACCAAGAAGGAACCTGTTGGGGTCTGTGGTATTGTCATCCCCTGGAACTATCCCTTAATGATGCTGTCCTGGA | | |
| | | AGACAGCAGCCTGCCTGGCTGCCGGGAACACCGTNTTGATCAAGCCTGCCCAGGTGACCCCACTCACAGCCTTGAA | | |
| | | GTTTGCAGAGCTGACACTGAAGGCTGGCATTCCCAAGGGTGTGGTCAACATCCTCCCAGGATCNGGCTCGCTGGTT | | |
| | | GGCCAGAGACTCTCAGACCACCCTGATGTGAGGAAAATAGGGTTNACAGGCTCCACGGAGGTGGNAAAACACATCA | | |
| | | TGAANAGCTGTGCCCTGNGTAATGTNAAGAAGGNCTNCCTGCAGCTGNNNNTAAAGNCACCCTTNTCATCNNTCCTG | | |
| | | CTGNNNCCTN | | |
| | | gb\|M59861.1\|RAT10HCO Rattus norvegicus 10-formyltetrahydrofolate dehydrogenase mRNA, complete cds | | |
| | | Length = 3109 | | |
| | | Score = 555 bits (280), Expect = e−156 | | |
| | | Identities = 343/367 (93%) | | |
| | | Strand = Plus/Plus | | |
| Clone # | T7 sequence | GGGGGTGCGNGGNGGNGGGCCGTCCCCNGCCTNCCCTNCGNGCGCCGGGTTTCGCCCCCGCGGNGTCNNNCCCCNN | | |
| 124 | | NGANTNTACNNCNCGACGAGTNTGAGGGCCGCTGCNGTGAGCCTTGAAGCCTATGGCGCGGGCCCNGNTGGAGCCG | | |
| | | CCGNAGGTGCATATCTTGNTGGTANTAGCAAATATTCANACNATAACTTTGAATGCCNAAGTGGANAANGGTTCCAT | | |
| | | GTGAANAGCAGTTGATCATGGGTNANNTNGGTCCTGANAGATGGGCGAGTGCCGTTCCGAANGGACAGTCGATGGC | | |
| | | CTCCGATGACCTCGGNCGATCANAAAGGGAGTCNGGTTTANATCCCCGAATCCNGAGTTGGCNGANATGGGCGCCN | | |
| | | TGAGGCGTACAAATGCGGTAACNNGACCGATTGCTGAGAANCCNNCNNGAGNNNCGGANAGAGNTCTNTTTTNTTTG | | |
| | | CGAAGGGCATTNGCGCCCTGNAATGGNTTCAGNNCCTANAGAGGGNTCCNTGCCTTGTAAAGCGTCGCNGTTCCNG | | |
| | | ANGNGTTTCTNGTGAGCTCTNCTNNGCCCTTGNAAATCCCGTGNNANAGGGTGTAAATTCTNTGTCNCGGGCNCNAA | | |
| | | CNNATATTNCTTATGCATGATCNTCNAANNTTTAACCANCCTNATGGCCATGTTNGCNANAAATTGNACCTCTAANGT | | |
| | | TAAAGTTCCNNCNATTNCCTNNANNCCTTNTAANCTTTNNNNGNAATAAATNGATTTGGNCCTTTTTAAATGGGCCTT | | |
| | | NGGGTCCNGNNNCCGGNANNTTTGNGNTGCNNTCCNAAAAACCCNCATGGNNTTNGNAACNTTCCNCNNNNCCCNNTN | | |
| | | NTNTTTTNTAACNTAANTGGTNGTGCTNCTCNCNCTTCTTTCTTTANNC | | |
| | BGH | TGCAGGGAAGACAGAAAGGCNTCCCAGGCCACTTGGTTTATTAGATCCTGAAGAGAGGTGTAGGCAGTGCCCCTGG | | |
| | sequence | GNCGCTGCCACCTCCTGGGGGAGGACCTGTGGGAGGCACAGGGCCGAACCTCGTTTTGATACACACAACCCCATTT | | |
| | | GAGGGAAAACAGGCTGCTTCGAAGCCTGAGGGATGGNGAGGGGTGATGCCTGCCATACAGGAAGCCAGGTCCTGG | | |
| | | GAGGGCACAAACGATGAATCCATCACTGCCCCAGCTCTGCCAGCATGCCCACCTGGCCCTGGGGAAGCCAGGCAAG | | |
| | | GGAGGGCACAGGCGTNTGAGGGACACAGACAGNTNCTGGNGACGGNAGNATCTGNTGAGCAGGAGGTTTCAGCAN | | |
| | | ANCTGANCATTANAGCANNCAAGGCTNNATATAGNANGTGCNCTNGNNAACCTNANNCNNTTTNTCTGNACTNNANGN | | |
| | | NCTGNCTTCTGNNGNNGATCNGNNCTNNNTNCTNTNCCNTNTNNNTNAGG | | |
| | Total | Mouse 285 ribosomal RNA | | |
| | cDNA, | Length = 4712 | | |
| | EST cluster | Score = 333 bits (168). Expect ' 7e−89 | | |
| | | Identities = 320/380 (84%), Gaps = 4/380 (1%) | | |
| | | emb\|X00525.1\|MMRNA02 Hydrolagus colliei internal transcribed spacer 1, partial sequence; 5.8S ribosomal RNA gene and internal transcribed spacer 2, complete sequence; and 28S ribosomal RNA gene, partial-sequence | | |
| | | Length = 4463 | | |
| | | Score = 218 bits (110), Expect = 3e−54 | | |
| | | Identities = 249/300 (83%), Gaps = 3/300 (1%) | | |
| | | Strand = Plus/Plus | | |
| | | gb\|AP061799.1\|AF061799 Squalus acanthias internal transcribed spacer 2; and 28S ribosomal RNA gene, partial sequence | | |
| | | Length = 3650 | | |
| | | Score = 216 bits (109), Expect = 1e−53 | | |
| | | Identities = 229/274 (83%), Gaps = 2/274 (0%) | | |
| | | gb\|AF061800.1\|AFC61800 | | |

TABLE 2

| | |
|---|---|
| Clone 0001-T7 Sequence | |
| AGCTCATGTTGGACAGTCGTGTGAGGAGCTATGGAGCACAGCAGTAATCGCCCAGAGGACTTCCCGCTTAACGTGTTCTC | |
| TGTCACTCCGTACACACCCAGTACCGCCGACATCCAGGTGTCCGACGACGACAAGGCAGGGGCCACTTTGCTTTTCTCAG | |
| GCATCTTTCTAGGACTGGTGGGGATCACTTTCACTGTCATGGGCTGGATCAAATACCAAGGTGTCTCCCACTTTGTANAT | |
| GGACCCAGCTCCTCGGACCCATCCTTCTGTCGGTCGGAGTGACATTCATCCTGATCGCTGTGTGCAAATTCAAAAYGCTA | |
| TCCTGCCAGTTGTGCTCAGATAACGAGGAGAGGGTCCCGGGACTCCGGACCAGGACTTCCSGAGGACAGTCGTTCGTTTT | |
| CACTGGCATCAATCAGCCCATCACCTTCCACGGGGCCACCGTGGTGCAGTATATCCCTCCTCCTTACGGTTCTCAGGAGC | |
| CCCTGGGAATGAACGCCACCTACCTGCAACCCATGATGAATCCTTGCGGTCTCATACCTCCTAGTGGAGCAGCGGCTGCC | |
| GCACCAAGTCCCCCTCAGTACTACACCATCTACCCTCAAGACAATGCTGCGTTCGTNGAGAGNTGAGGGCTTCTCTCCTT | |
| TCGTGGGCACTGGATATGACAGGCCCGACTCTGATGCTGACCAGCTAGAAGGGACGGAGTTGGAAGAGGAGGACTGCGTA | |
| TGTTTCTCTYCTCCACCGTATGAGGAGATATACGCTCTACCTCGCTAGAGACTGCAATGCTAAGGGGACGGACATTTAAG | |
| CCCCTGNGATGTGATACTTGGAGAGTTTATCGCTGAGTTCTTCAGAAGTTAGGTGTCAAAGCAGCTNAGGAGATCTTACA | |
| GATGTCATTNAAGGNGGGAAAGAAGTGCCCNGAGACTGCTAAATTAAGCTGCCCTGGTTAAATTCCCCTCTGCTCTGGTT | |
| TTGAATTCTCTCAGCTAAGAAACCCTCTGCAGCTGGAGAGTCGCTCTGAGATAGAGAGATTTNGGAGCCCACGCAGNGCC | |
| T | |
| Clone 0001-BGH Sequence | |
| ATAGACATGTCTAGTTTTTTATTACTAGTTATCATCCAAGTGAAATGTCCCTGAGGCATAATATGAATCACAATAATAAA | |
| CCAAGATTGTTTTGCTGATATCCTTGAAAAACCTGGACTCTTCTGACAGAGAGTAAAAGCAATCCCACATATAAGCACAG | |
| TACCAAAACCTTCAAGACCTGACAATTCCTGTATCTCTCTTGGGGGCAGCCACCTTTACCTTGAGTGGCCTTTGATCTTT | |
| GTAAAACTGTTTTCTTTCTAATTCTTTGGACACCTCCTAAAGTCTGCAGCTTTGGGCTGNGGTTGGTGACACCGTTCAGA | |
| CTTAGGAAGCTGAAGGCTACCAGCTTTTCTTTGTTCAAATGAAGGTGAGAGCTACGCCCGGGGGCGGAGGKAGRGGGAGA | |
| CGGGATGACTGATCATCTCCATGATTCATGACTACAAAGCGACACGGCAGNATGGCTCTGNGGAACAGATCAGAGGCATA | |
| CCTGTGTTCCATAAGCNACTCAGCTGTACCTGCGTTCTGTAAGCCACTCGGCTGAGCTAGTTCGNGCCTGGGAATCACAG | |
| CCTGGGGGNGGGCAGAGGGAGCAGGCGCTNACCNATGTACATTACGCCTTGAACTTCANCCTTGCAAAGGAAAACNATCA | |
| NGAGCAGGGGTCACTNTAACTCGGNGGACAGCACATGGNGACATTCATTATCCTCCTGNTACCTCGTCCACAGTAGGATC | |
| GGCCACCCAACACTATTCTAACATGGGGCACTGTTTCATCCNACTCTACTTATTCATTTATTTCTTAATGGATTTTATTT | |
| CTTTAACAAACTCTTNAAGATCCAAAGGCTTCCAGNGAGACCANATAANAGTCNAAGTTGTCTAANATAAGTNACTCTGC | |
| TGCGGAAGGTTCAGNGCCGTGCAGGGAAATTTCTACCTGAGCCTGCTCTCTTCCCTGCTTGCTTGCCAGCCTCCCTTTNA | |
| TCANTCTNAGAGCTGCCATGGCTGCCCGGATTTAAAACTAANACAATCTTTNAAAACTAAGATNTCCTTTAAATGAATAT | |
| TCCGCATGG | |
| Clone 0002-Contig | |
| CGCCGCCTGAGCGCCCGGCCCGACCCCGCCATGGGGTGCTGCTATAGCAGCGAAAACGAGGACTCGGACCAGGATCGGGA | |
| GGAGAGGAAGCTGCTGCTGGACCCCAGTAGCACCCCTACCAAAGCCCTCAATGGAGCCGAGCCCAACTACCATAGCCTAC | |
| CTTCAGCTCGCACAGATGAGCAGGCCCTGCTTTCCTCCATCCTTGCCAAGACAGCTAGCAACATCATTGATGTGTCTGCC | |
| GCAGACTCCCAGGGCATGGAACAGCATGAGTACATGGACCGGGCAAGGCAGTACAGTACCCGCTTGGCTGTGCTTAGCAG | |
| CAGTCTGACCCATTGGAAGAAGCTGCCACCGTTGCCATCTCTCACCAGCCAGCCCCACCAAGTGCTGGCCAGTGAGCCTA | |
| TCCCCTTCTCTGACTTGCAGCAGGTCTCCAGGATAGCTGCGTATGCCTATAGTGCACTTTCTCAGATCCGCGTGGATGCG | |
| AAAGAAGAGCTGGTTGTACAGTTTGGGATCCCATGAAGAGAGGGGCCCTAGGACAGCTCTTCCCTCGTCTTCACCCCGTC | |
| TCCACCCCACCTCTTCTGGCCCCCAGCCTCACTGTGGCTCTCTACAGTACCTAACCTGCTACTAATCACGGAGAAGAATG | |
| TGGAGGGAAAGAACAAGGCTGGAGGCCGGAGCAAGTGAGGACTAAGCAAGGGAAGGGAGGACCGATTGCCATCGGCCTTC | |
| ATGCTCTGGTTAGGGTGAGGTTGGGGCCAAGAGGACAGGGCCTGGCAGATCTTCAGTCATTGGGAAGATGGAGATACCNC | |
| TGTAGGGGTGACACCGGGAGACCTAGCAGATCCCTTCCTGCCCTCTTTCTCTTGGCCTCCGATTCACTCCTGTCCCCTTC | |
| CCTGACTTGGTGCTCACAGGCACCTCACTGGGGATTATGACCAGGGTCTAGACGAGCTTGAGTCTGAATTGAGTTTGTAT | |
| TTCTACACCCTGGGTTTTTACATGTTTGCTCTTTTTGTTTTTGTTTGTCACCCCTCGATAAAGAAAGTATATTCATTTG | |
| AAAAAAAAAAAAAAAAAAA | |
| Clone 0003-T7 Sequence | |
| AGGTTCCTGTCGTTTATGCTGCGCGCCGCGAGCATCTTCGTGAAGGACGCTGTGCTCTASTCTGGCTTCACGCTGGATGA | |
| GGCCGAGCGCCTCACGGAGGAAGAGTTGCATATCATCGCGCAGGTGCCGCCTCCACCCGCCGCTGCCGCCGTTGGCTACG | |
| CTGGCTGCCGTGTGGCTGTGACCTTCTTCCTCTACTTCCTGGCTACCAACTACTACTGGATTCTGGTGGAGGGACTGTAC | |
| TTACACAGCCTCATCTTCATGGCCTTTTTCTCAGAGAAGAAGTATCTGTGGGGCTTCACCATCTTTGGCTGGGGTCTGCC | |
| GGCTGTCTTCGTGGCTGTGTGGGTCGGTGTCAGAGCAACCTTGGCCAACACTGGGTGCTGGGACCTGAGCTCTGGGCACA | |
| AGAAGTGGATCATCCAGGTGCCCATCCTGGCATCTGTTGTGCTCAACTTCATCCTCTTTATCAACATCATCCGGGTGCTT | |
| GCCACTAAGCTTCGGGAGACMAATGCGGGCCGGTGTGACACCAGGCAGYAGTACCGGAAGCTGCTCACGTCCACGTTGGT | |
| CYNGCGCCACTCTTTKGTGTCCACTACACCCGTCTTCATGGCCTTGCCGTACACCCGAGGTCTTAGGGACACTGGGGCA | |
| ATCCAGATGCACTATGAGATGCTCTCTTTAACTCCTTTCAGG | |
| Clone 0004-Contig | |
| AGGCGCTGCCTACCAGAGCGCAGCATGACGGCCATCGGCGCGCAGGCCCACAAGCTGTTGGGCCTTAAGAGGCCCCACCG | |
| GTCTTTCTTTGAGTCCTTCATCCGGACACTCATCATCGTGTGCACTGCCCTGGCTGTGGTCCTTTCTTCAGTCTCCATCT | |
| GCGATGGCCACTGGCTCCTAGTGGAGGATCATCTCTTTGGGCTGTGGTACTTCTGCACCATCGGCAACCACAGTGAACCA | |
| CACTGTCTGAGAGACCTGAGCCAGGCCCATATGCCCGGGCTGGCTGTAGGCATGGGCCTAGCACGCAGTGTGGCCGCCAT | |
| GGCAGTGGTGGCTGCCATCTTCGGCTTGGAGATGCTCATTGTGTCCCAGGTGTGTGAAGATGTCCGCTCACGGCGCAAGT | |
| GGGCCATCGGTTCCTACCTCCTCCTGGTTGCCTTTATCCTCTCCTCTGGGGGCCTCCTCACCTTCATCATCCTGCTCAAG | |
| AATCAGATCAACCTCCTGGGCTTCACCCTGATGTTCTGGTGTGAATTCACTGCCTCCTTCCTCTTCTTCCTCAATGCCGC | |
| CAGCGGCCTTCACATCAACAGCCTCACTCAGCCCTGGGACCCTCCAGCAGGGACCCTGGCTTACAGAAAGCGAGGTTATG | |
| ATGGGACTTCTCTGATATAGGACCTCTGGCTGAGATTGAATAGGACAACCAATGACCTTGACACTGCCTCTTAGGCACTT | |
| AGCTCTAGCAATGCCCTGGAAGTCTCTTCAGCTGAGCTCCAGGGCAAAGGCAGAAGGGTGCCTCTGTACGACGGCACAGT | |
| GAGCTGGATAGGTTAGTCATGCTATTAAAATCTCAATTTGAAAGTAAAAAAAAAAAAAAA | |
| Clone 0005-Contig | |
| AGCAGACTCAGGAAGAAACCATGGTGCTCTCTGGGGAAGACAAAAGCAACATCAAGGCTGCCTGGGGGAAGATTGGTGGC | |
| CATGGTGCTGAATATGGAGCTGAAGCCCTGGAAAGGATGTTTGCTAGCTTCCCCACCACCAAGACCTACTTCCCTCACTT | |
| TGATGTAAGCCACGGCTCTGCCCAGGTCAAGGGTCACGGCAAGAAGGTCGCCGATGCTCTGGCCAATGCTGCAGGCCACC | |
| TCGATGACCTGCCCGTGCCCTGTCTGCTCTGAGCGACCTGCATTGCCCACAAGCTGCGTGTGGATCCCGTCAACTTCAAG | |
| CTCCTGAGCCACTGCCTGCTGGTGACCTTGGCTAGCCACCACCCTGCCGATTTCACCCCCGCGGTGCATGCCTCTCTGGA | |
| CAAATTCCTTGCCTCTGTGAGCACCGTGCTGACCTCCAAGTACCGTAAGCTGCCTTNTGCGGGGCTTGCCTTCTGGCCAT | |
| GCCCTTCTTCTCTCCTTGCACCTGTACCTCTTTGGTTTTGAATAAAGCCTGAGTAGGAAGAAAAAAAAAAAAAAA | |
| Clone 0006-T7 Sequence | |
| AGGGGCTTCGGACCCGGAAGTGGCGCCTTGGGCTCCCGGCGGCGCCGCGGGGATGGCGGGAGCCGGAGCTGGTGCAGGAG | |
| CTCGGGGCGGCGCGCCGGCCGGAGTCGAGGCCCGCGCTCGGGACCCGCCACCCGCGCACCGCGCGCACCCTCGCCATCCT | |
| CGGCCCGCGGCTCAGCCGTCGGCGCGCAGGATGGACGGCGGCCCGGGCGCCCCGGGCTCCGGGGACAACGCCCCGACCAC | |
| CGAGGCGCTGTTCGTGGCGCTGGGCGCGGGCGTGACGGCTCTCAGTCACCCGCTGCTCTACGTGAAGCTGCTGATCCAGG | |
| TGGGTCATGAGCCGATGCCCCCCACCCTTGGGACCAATGTGCTGGGGAGGAAGGTCCTCTACCTGCCGAGCTTCTWCAAC | |
| CTATGCCAAGTACATTGTGCAGGTGGATGGGAAGATAGGGCTCTTCCGGGGCCTGAGCCCCCGCCTTATGTCCAACGCCT | |
| TGTCCACTGTGACCCGCGGCAGCATGAAGAAGGTTTTCCCTCCAGATGAGATGGAGCAGGTTTCCAAACAAGGACGACAT | |
| GAAGACCTCACTCAAGAAAGTTGTGAAGGAGACATCGTATGAGATGATGATGCAGTGTGTATCGCGAATGCTGGCCCATC | |
| CCTTACACGTGATCTCGATGCGATGCATGGTGCAGTTTGTGGGACGGGAGGCCAAGTACAGTGGTGTGCTGAGTTCTATT | |
| GGGAAGATCTTCAAGGAAGAGGGGCTGCTGGGATTCTTCGTTGGCTTAATCCCTCACCTCCTGGGCGATGTGGTTTTCTT | |
| GTGGGGCTGTAACCTGCTGGCCCACTTCATCAATGCCTACTTGGTGGACGACAGCTTTAGCCAGGCCCTGGCCATCCGGA | |
| GCTACACCAAGTTTGTGATGGGGATTGCAGTGAGCATGCTGACCTACCCCTTCCTGCTCGTTGGAGATCTCATGGCAGTG | |
| AACAACTGTGGGCTGCGGGCTGGACTCCCTCCGAATTCCCCTCAGTTCAAGTC | |
| Clone 0006-EGH Sequence | |
| GAGCAGTGTTGGGGTGGCATCCCCCTTCCTGCCTAGAGGTACTGGAGTCCATCTTGTACTCAGGCAGAGGCAGGCTGCAG | |
| AGGCAAACGTCACTCAGTGCCAACGCTTCCCTGCACCTCTAGCCCAGCTCATCCTGCCAGTCAGCCAGAAGCACCCCCGC | |
| CCCCCACTTCCTGCTTTGTAAATTGGGCGCCATCACACCTGGGCCATGGGAGGCTGGCGCTATGTTCCCAACACTAATTT | |
| TCTTATACAAGGGTGGTGCCTTCTCCTGAATAGGAAATCATGTTCTCCTCAGACCATCCCCTCATCTGCTTGTCTGTGCT | |
| GGTGACGCCAGGTGTGAGGGTTCAGTCACTGTGCTGGGTGCGAATACGCACAGGTTACATAGGCCGACATCTAGTCCTCC | |
| CCTCGTGGTAAGATAGACCCATCTCCTCGAATAAATGTATTGGTGGTGATTTGGAAAAAAAAAAAAAAAAAA | |
| Clone 0007-Contig | |
| AGCTCCGCCCCTGCTACTGGACCATGGARACTGTGGCCCAGTAGAGACCTTAGTGTGAGGCTTTCAGGGGCGGCGGCCAT | |
| GGAGGCCGTGCTGAACGAGCTGGTGTCTGTGGAGGATCTGAAGAATTTTGAAAGGAAATTTCAGTCTGAGCAGGCAGCTG | |
| GTTCTGTGTCCAAGAGCACGCAATTTGAATATGCCTGGTGCCTGGTTCGAAGCAAATACAATGAGGACATCCGCAGAGGC | |
| ATCGTGCTGCTGGAGGAGCTGTTGCCCAAAGGGAGCAAAGAGGAACAGCGGGACTATGTCTTCTACCTGGCCGTGGGCAA | |
| CTACCGGCTCAAGGAATATGAAAAGGCTCTAAAGTATGTGCGAGGGCTGTTGCAGACTGAGCCCCAGAACAACCAGGCCA | |
| AGGAGCTGGAACGCCTGATTGATAAGGCCATGAAGAAAGATGGACTGGTAGGCATGGCCATCGTTGGTGGCATGGCCCTG | |
| GGCGTGGCAGGCCTGGCTGGACTCATTGGACTGGCTGTCTCCAAGTCCAAATCCTGAAGGCAGCCTCACCTGCTCTCTGC | |
| CCCGGGACGCCTAGGAGCCTGGGGGACACTGGAAGAGGGGCCTGTCCATCCTCACCATCGCCTTCCCTTTTCTCCTGCAC | |
| CCCTGTAGTCTACCTCTACAGTCTCCATGACCCCCAGCCTYTTAGCCCCTGCACCTGTCGTTTAACCCTGTCATGCTTTG | |
| CAATGAGTGTAAATAAAATTGGGCCGTGGCTCGGGAAAAAAAAAAAAAAAAAAAAAAAAAAAAAAA | |
| Clone 0008-Contig | |
| AGATTGACTTGGGCACTGACATGCTTCCTGCCATCTCCCTGGCCTACGAGCAAGCTGAGAGCGACATCATGAAGAGGCAG | |
| CCCAGAAACCCCAAAACGGACAAACTTGTGAACGAGCGTCTGATCAGCATGGCCTATGGACAGATCGGTATGATCCAGCC | |
| CCTGGGAGGCTTCTTCACTTACTTTGTGATTCTGGCTGAGAACGGTTTCCTGCCCTTTCACCTGTTGGGCATCCGAGAGA | |
| CCTGGGATGACCGCTCGGTCAACGATGTGGAGGACAGCTACGGGCAGCAGTGGACCTACGAGCAGAGGAAGATCGTGGAG | |
| TTCACCTGCCATACAGCGTTCTTTGTCAGTATTGTGGTAGTGCAGTGGGCCGACTTGGTCATCTGCAAGACCAGAAGGAA | |
| TTCTGTCTTCCAGCAGGGAATGAAGAACAAGATCTTGATATTTGGCCTCTTTGAAGAGACAGCCCTTGCTGCTTTCTTAT | |
| CCTACTGCCCCGGGATGGGGGCAGCCCTTAGGATGTATCCCCTCAAACCTACATGGTGGKTCTGTGCCTTYCCCTACTCC | |
| CTTCTYATCTTTGTGTATGACGAGGTGCGGAAGCTYATTATYACGCGGSGCCCTGGCGGCTGGGTGGAGAAGGAGACCTA | |
| CTACTAGCCCYACTGCCCTGSACGCCGKGGAACATTGTGCCACACACTGCACCCACCCCWACCCCCTCTWYSYSYMCTTY | |
| AAGTYTTGGAGCTCGGRACTCTWCCCTSRWAGGRWWKYWSSRARSCMTKTGGGRATKYWKACSYCYTGGRATGRAGSAWR | |
| TAGCTGYAMCSSRRRRCSGKKYGKKGGGGAGGRYTGCCTGAGAAACNCCGTGTMCKKGGACGACAKCGGRMAAGRATTKW | |
| TACGTGCCTTTTTGTTTTTGTAAAAAAGGAAAACCTGGAAAGACTGAAAGATTACGTTTTATATCTGGATTTTTACAAAT | |
| AAAGATGGCTATTATAACGG | |
| Clone 0009-T7 Sequence | |
| AGCCGGAGGCCGAGCCCAGTCGCCAGCTCCTGCTCTGCTCCTCTCCCGCCTGCCGCCGCGCTGCACGCCTCGAGCACTCC | |
| CTCGGCCCCGCCGGGGACCGGGGACCCCGCAGCTACCGCCATGCTGCCAGTGCTCTACACCGGCCTGGCGGGGCTGCTGC | |
| TGCTGCCTCTGCTGCTCACCTGCTGCTGCCCCTACCTCCTCCAAGATGTGCGGTACTTCCTGCGGCTGGCCAACATGGCC | |
| CGGCGGGTGCGCAGCTACCCGCACCGGCGACCCGTGCGTACCATCCTGCGGGCCTTCCTGGAACAAGCGCGCAAGACCCC | |
| ACACAAGCCCTTCCTGCTGTTCCGAGACGAGACGCTCACCTACGCCCAGGTGGACCGGCGCAGCAACCAAGTGGCGCGGG | |
| CGCTGCACGATCAACTGGGCCTACGACAGGGGGATTGCGTAGCCCTCTTCATGGGCAATGAGCCGGCCTACGTGTGGATC | |
| TGGCTGGGACTGCTCAAACTGGGCTGTCCCATGGCGTGCCTCAACTACAACATTCGTGCCAAGTCTCTGCTGCACTGCTT | |
| TCAATGCTGCGGGGCGAAGGTGCTGCTGGCCTCCCCAGATCTACAAGAAGCTGTGGAGGAGGTTCTTCCAACCCTGAAAA | |
| ANGATGCCGTGTNCCGTCTTTTACGTAAGCAGAACTTCTAACACAAATGGTGTGGACACAATACTGGACAAAAGTAGA | |
| Clone 0009-BGH Sequence | |
| TTTTTTTTTTTTTTTAGTGTTAATATAGTTTATTATGTCTTTAAAAAAATAAGGCCCTCTCTCCAAGAAGCTTAGTTTGC | |
| AAGGACAAATGGCAGGTGCACATTGAAAAATAATTGTTTCTAAATCTTTTTACTTGCAAAGGTTCAGGTGTAATTTAAAA | |
| AAAAAACAAACAAACTATCCTTTTAATGAATAATTTCCTAAAAATAAAATCGCACCTTATAGCCTTCAATCAAGTTAAAG | |
| TTGGATTCTACGTATGAAGTGGCTCTGCGAGGTCTATCGAGTTTCTTTCTGGAAATGTCATGAGCTAAACCACCAGGGAA | |
| TATTCAGAGCTTCAGAGTTTTATCAATTATGCCATTATAAATGTTCTCAGTCATGGGCACAAATGTTTTCTCTCCATCAT | |
| CCATGAAATACAAGGTATCTTTGATGACTGTGGGATTGAAGCCCTCTTCCATCAGGGTCACTTTGCGGTGTTTAAAAGTC | |
| CCAGTGATCTCAATGGTATCTTGTATCCTCAGGAACCGAGGCCTCGCGTAACTGGGCACCTACTCCGCGATGTGTTGAAA | |
| GAGTTTCTTTCCATTGAACTCGTAGTTTTCTTTGATCTTGA | |
| Clone 0010-Contig | |
| AGCGCGGGAGGCGCATGGCGGGCATGGCGCTGGCGCGAGCATGGAAGCAGATGTCCTGGTTCTACTACCACTACCTGCTC | |
| GTCACTGCGCTCTACATGCTGGAGCCCTGGGAGCGAACCGTGTTCAATTCGATGCTGGTTTCCGTGGTGGGGATGGCCCT | |
| GTACACTGGCTACGTCTTCATGCCCCAGCACATCATGGCTATTCTGCATTACTTTGAAATTGTACAGTGACGAAGATGTG | |
| ACCAGGATCCAGAGGTTCCTGGGGAAGATCTGCCTTGTGAAGTTGGAATGAGACCTCATCAGATGTAAGATGTGCTACGG | |
| ATGTCCACGTGACCAACCTTATAAATACAAAGACTTTAAAAAAAACTTCATGAGTAGAACAGGAAAAATCATCCTGGCTC | |
| ATGTGTTGWGTTCTTTCTTTTTGATTTTAACAGAGGCTCTTATATAGTAGCTTTTATCTATTTTAACATTGTAGTCATTT | |
| GTACTTTGATATCAGTATTTTCTTAACCTTTGTGACTGTTTCAATATTATCCAGTGAAAGCTTTTCTTAATGTAACTTTG | |
| AGTACATCTCAATTGCCTTCTATTTTTAAAACCTAAGGTCATTAGTTGGGCTTTACTGTTCTTGCTATCATATGGCATAT | |
| ACATCTGCCTGGATATATTTCTACTCTTGACCAAAGTTTTGTAAGAAACAATACAGAATTTGGGGACGGGAGGGAGGATA | |
| CTTTGAGAACTACTTACAAAAGATTTATCTGCTTGCTTGAACTCAGGAGTACGGTTTTAGCTCCCTAGACTCTTAACAGC | |
| TTTTGCTCTAAAACTATTAAAGTGTTTCTTAGTAATGAAAAAGTAAAGATCTTGCTAACATTAATACAAGGAACATTTCN | |
| CCTTTTAGATATTAAATTCTTATGTGGACTTATTTCCAACCAACTTTGATGATTCCTGTGATTCGTGATTGGTGGCCGGC | |
| TTGTATGACATATGCTGAGATACACTGTGGAGCTTTTCATACAAGGCTTATCAGATCCTCTGAGGCAGTATTGACCCTGT | |
| ATACGCTCAAGAGTTAATTTGATGTTGTAACAGGCGTTGTGAAATGCATTTGCACGTTGTGATAAAGACMCAATGAGATG | |
| AATTGTGCAGAACCATAACCAAAAGAAATGTAAACTTGCTTAAAAATCCTTTCACTCTTTGTATTTTTTTTAAAACGGTT | |
| TTTATTCCTTAGATGTAAAATGACTACTTTTTTTTGATGTAAACTCATTAAATTCAAAGAAAAATGTAAAAAAAAAAAAA | |
| AAAAAAAAAAAAAAAAAAAAAA | |
| Clone 0011-Contig | |
| AGCGAGTTTGCAGACTTCTTGTGCCCAGCTAGCCGCCTNAGGTGTTNGAACCATGAATCTTTTACTCCTTTNGGCTGTCC | |
| TCTGCTTGGGAACAGCCTTAGCTACTCCAAAATTTGATNAAACCTTTAGTGCANAGTGGCACCAGTGGAAGTCNACGCAC | |
| AGAAGACTGTATGGCACGAATGAGGAANAGTGGAGGANAGCGATATGGGAGAAGAACATGAGAATGATCCAGCTACACAA | |
| CGGGGAATACAGCAACGGGCAGNACGGCTTTTCCATGGAGATGAACGCCTTTGGTGACATGACCAATGAGGAATT | AGGC |
| AGGNGGNGAATGGCTATCGCCACCAGAAGCACAAGAAGGGGAGGCTTTTTCAGGAACCGCTGATGCTTAAGATCCCCAAG | |
| TCTGNGGACTGGAGAGAAAAGGGTTGNGTGACTCCTGTGAAGAACCAGGGCCAGNGCGGGTCTNGNNGGGCGTTTAGCGC | |
| ATCGGGTTGCCTAGAAGGACAGATGTTCCTTAAGACCGGCAAACTGATCTNACTGAGTNAACAGAACCTTGTGGACTGTT | |
| CTCACGCTCAAGGCAATCAGGGCTGTAACGGAGGCCTGATGGATTTTGCTTTCCAGTACATTAAGGAAAATGGAGGTCTG | |
| NACTCGGAGGAGTCTTACCCCTATGAAGCAAAGGACNGGWTCTTGTAAATACAGAGCCGAGTTCSCTGTGGCTAATGACA | |
| CAGGGTTCCGTGGATATCCCTYAGCMAGGAGAAAGCCCTCATGAAGGCTGTGCGACTGTGGGGCCTATTTCTGTTGCTAT | |
| GGACGCAAGCCATCCGTCTCTCCAGTTCTATAGTTCAGGCATCTACTATNAACCCNACTGTAGCAGCAAGATCCTCGACC | |
| ATGGGGTTCTGTTGNTNGGCTATGGCTATNAAGGANCAGATTCAAATAAGAATAAATATTGGCTTGTCAAGANCAGCTGG | |
| GGAAGTGAATGGGGTATGGAAGGCTACATCNAAATAGCCNAAGACCGGGNCNACCTCTGTGGACTTGCCNCCGCGNCCAN | |
| CTATCCTGTCNTNAATTNATNGGTAGCGNTAATGAGGACTTATGGNCTCTATGTCCNAAGGAATTCAGCTTAAAACTGAC | |
| CNAACCCTTATTNANTCNAACCNTGGTNCTTNAATCATTNAGGATCCAANTCATNATTTNAATTCTGTTGCCATTTTTAC | |
| ATGGGTTAAATGTTACCNCTACTTAAAACTCCTNTTATAAACANCTTTATAATATTGAAAACTTAGTGCTTAATTCTNAG | |
| TCTNNAATATTTNTTTTATATAAAGGTTGTATAAAACTTTCTTTACCTCTTAAAAATAAATTTTAGCTCAGTGTGTGTGT | |
| AA | |
| Clone 0012-Contig | |
| GAGCGCCGCCGGGTCGGCCAGCGAGGCCTCAGAGGTTCCAGACAACGTGGGAGACTGGCTCCGCGGCGWCTTCCGCTTCG | |
| CCACCGATCGAAACGACTTCCGGAGGAACTTGATCCTYAATTTGGGACTCTTTGCTGCGGGAGTCTGGCTGGCCAGGAAC | |
| TTGAGTGACATTGATTTGATGGCCCCTCAGCCAGGGGTGTAGCCAGAGAATGGAACTCCTGTGTATTCAGACTTTCCAAA | |
| GACAGCCTACTGTCTGTGACCACAAGATCCTACCTGAGTGGCAGCTGAAGTTGACTCCCTCTCCKTGCCTGAACCCCCCC | |
| CCWCTGCCCCCCCATCCCCCAGTGTCGGCTRAGATGTTGCCTCTGCACGGTTCTGTGTGCAGTTCCCNACTTTCTGCAGA | |
| AGATGGTCCTTGCCCTTGTCCTNAAGAGTAATAATGGTTCTTGAAAAAGATTTCAAATAAAGCCTGCNCAT | |
| Clone 0014-T7 Sequence | |
| AAAGGAAAAACACAGCTCAGCAGATCCAGGCACTAAAGAGAGCTAGCTGCAAGCAGGAGCAGTCAAGAATCTGTGGTCAG | |
| AAGTACTGGAGTGGGCCAGCAGGGCCAGCTTTTTCTACCATGGCAGCCCAAGGCTACGGCTACTATCGCACTGTCATATT | |
| TGCGGCCATGTTTGGAGGCTACAGCCTGTACTATTTCAACCGCAAAACCTTCTCCTTTGTCATGCCCTCCTTGGTGGATG | |
| AGATCGCTCTGGACAAGGACGATTTGGGGCTCATCACAAGCAGCCAGTCGGCAGCCTACGCCATCAGCAAGTTTGTGAGC | |
| GGGGTTCTGTCAGATCAGATGAGCGCCCGCTGGCTCTTCTCCTCTGGGCTGCTCCTGGTTGGTCTGGTCAACGTAGTCTT | |
| CTYATGGAGCTCCACAGTGTCAGCCTTWGCTGCTCTTTGGTTTCTTAATGGTCTGGCACAGGGGCTGGGCTGGCCCCCCT | |
| GTGGGAAGATCCTGAGGAAGTGGTTTGAGCCATCCCAGTTTGGCACTTGGTGGGCTGTGTTGTCAACCAGCATGAACCTG | |
| GCTGGAAGTTTGGGACCTATCTTGGCAACGATCCTCGCCCAGAGCTACAGCTGGCGCAGCACACTGGCCCTGTCTG | |
| Clone 0014-BGH Sequence | |
| TTTTTTTTTTTTTTTTTTTTTTTTTTTTTTTTTTTTTTTTTTTTTTTTAGDAACAAATCTAGCTTTATTGATCTATACAA | |
| GKCGGTTTGCCCTCTATATTCTTCCCTTTTGKCCCAGGGTATTCATGAGAGGGGGGGACYCCTYTYCTCCCTTAGGGACT | |
| CRGGTATTGGCAACAATTTTGCCCCCTGCCAAGAAGCCTTGGGGTACAGCAGAAACCACAGGCCATTAATACTCACTAGG | |
| AGATCAGGACCTAGGAGCAAGAAGAAGGGTATAGGAGACACTCTGAATTCAGGAGTTGCCTGTCTGCCAGAAGGKAGAAA | |
| CAGAAGCTGAWGGAGTCTAGAACCAAACATCATCATTTTAAATAGAGCATGGGAAGGGAGT | |
| Clone 0015-T7 Sequence | |
| AGCGGAACTCAAGATGGCTGCGCTCTTGCTGAGTGCTGCTCCTTTGGGAACCACAGCTAAGGAGGAGATGGAGCGGTTCT | |
| GGAAGAAGAACACGAGTTCAAACCGTCCTCTGTCTCCCCATTTGACTATCTACAAATGGTCTCTTCCTATGGCACTGTCC | |
| GTTTGCCACCGAGGCTCTGGAATAGCCTTGAGTGGAGGGGTCTCTCTTTTTGGCCTGTCGGCACTGGTGCTTCCTGGGAA | |
| CTTTGAGTCGTATTTGATGTTTGTGAAGTCCCTGTGTTTGGGGCCAACACTGATCTACTCGGCTAAGTTTGTGCTTGTCT | |
| TCCCGCTCATGTACCACTCACTGAATGGGATCCGACACTTGCTATGGGACCTAGGAAAAGGCCTGGCAATACCCCAGGTC | |
| TGGCTGTCTGGAGTGGCGGTCGTGGTTCTTGCTGTGTTGTCCT | |
| Clone 0015-BGH Sequence | |
| CTTYTTTTTTTTTTTTTTTTTTTTAAAAKGAGTAGTACTTTAATCGTTTCCAAAGAAGATGGTACATGAATTTAGATTAG | |
| AAAATATGACATTAAAGGTGTATTTTCCTAGAAATGAAAAACAGAACAGAAACTTAATACAATTTTTTTTGATCTTCCTC | |
| ATACATCAAAAGCTGTGTATCTGTTCCCAGGCTTGAGCTCCAAGCTCCCAGGCTGCTCTTTATCTCCCTCTAGCTGAGCA | |
| GGACAATCCCTCCTCCTCCACCTCTCTCCTCTCAGTCATGAGGTGTGGGTGCTAAAGGCCCTAAGCTGACCAATGGACAG | |
| AAGTGTCCTTTAAGCCAGCAGCCCACAGGCAGTGGCACTCGGGGCGGTCACAGAACTGGCATTGTTTMYACTGTCTCAGC | |
| AGAAAGCAGCGGAAAGCTGACCCTCAGCACAAATCAGTCAAATGTGGAACCGGAGCAAGGGCTCCCAGGCCCAGGGAGGA | |
| TGACCCTTTCGTACTACAACAGGTCTTTAAGCCGCTCTACTGCACTCCAAGGGGATTGAAAGCATGTGGCATCTAAACAA | |
| ATCAGGAGAACCTGGGTGGT | |
| Clone 0016-Contig | |
| GAGGGGAACAAGATGGCGGCGCCAAAGGGGAAGCTTTGGGTCCAGGCCCAACTGGGGCTCCCGCCGCTGCTGCTGTTGAC | |
| TATGGCGCTGGCCGAGGCTCGGGGACTGCAGCGGCCGAAGCCTTTGACTCGGTCCTGGGAGACACAGCCGTCCTGTCACC | |
| GGGCCTGTCAGCTGACCTACCCCTTGCACACCTACCCGAAGGAAGAGGAGTTATACGCATGCCAGAGAGGCTGCAGGCTG | |
| TTTTCAATTTGCCAGTTTGTGGATGATGGGCTTGATTTAAATCGGACCAAGCTGGAATGTGAATCTGCGTGCACAGAAGC | |
| ATATTCCCAACCTGATGACCAGTATGCTTGTCATCTTGGCTGCCAGGATCAGTTGCCATTTGCTGAACTGAGACAAGAAC | |
| AACTCATGTCCCTGATGCCAAGAATGCATCTCCTCTTCCCTCTGACTCTGGTGAGGTCGTTCTGGAGTGACATGATGGAC | |
| TCTGCACAGACCTTCATAACCTCTTCATGGACTTTTTATCTTCAAGCCGATGACGGAAAAATAGTTATATTCCAGTCTAA | |
| GCCAGAAATTCAGTATGCACCGCAGTTGGAGCAGGAGCCTACAAACTTGAGAGAATCATCTTTAAGCAAAATGTCCTATC | |
| TGCAGATGAGAAACTCACAAGCACACAGGAACTACCTTGAAGAGGAAGAAAGCGATGGCTTTTTAAGATGTCTATCTCTT | |
| AACTCTGGATGGATTTTAACCACAACCCTTGTCCTCTCGGTGATGGTGTTGCTCTGGATCTGTTGTGCAGCTGTTCCTAC | |
| AGCTGTAGAACAGTATGTTCCCCCTGAGAAGCTGAGTATCTATGGTGACTTGGAATTTATGAATGAACAAAAGCTGAGCA | |
| GATACCCAGCTCCTTCTCTTGTGATTGTTAGGTCTCAGACTGAAGAACATGAGGAGGCAGGGCCCCTGCCCACCAAGGTG | |
| AACCTTGCTCACTCAGAAATCTAAGCTTTTTAAAAGAGTCGTGGACACATAAACTTCCATTCCTCATAGAGCTTTTTAAG | |
| ATGGTTTCATTGGACATAGGCCTTAAGAAATCACTATAAAATGCAAATAAAGTTACCAAACTCTGTGAAGACTTTATTTG | |
| CTGTGACTTTACCTGTATTTTTCTAGTCATTTAAGATGGACATTGGGTTGTATTTTTATTTTACTAATATCTGTAGCTAC | |
| TTAGTTAGTTGCATTGGTTTTGGTTTTTTTCCTCTCTTCGCCAAATTCTATGAGCTGATCATTGTGGCCCCGCCCCTGCC | |
| ATGCCCCCCGTCAGTCATCTCACTTAATAACCGAAACCTTAGGGTGTGATGCTTCTGCCCGGAAATGGCCTCCAAACTGT | |
| CCTCTGGATTATAGCAGAAATGTTATTTAATGACWCTACATTTTCAGTTGTATTGAATTGAAATCATTAAAATCTATTTG | |
| AATAATTATGTTCTGAAAAAAAAAAAAAAAAAAAA | |
| Clone 0017-T7 Sequence | |
| AAAGGTACGAAGCTAGGGAAGATATTCGCGTGGCTAAATCTGCACGTGGAAGGAGCATTAACTTGGCCCTTTCTTATAGA | |
| GGACGGCAGGCCTTGAAAGCCATTGGTCTGGAAGATCAGATCGTTTCCAAAGGTGTGCCCATGAAAGCCAGAATGATCCA | |
| CTCTCTTTCTGGAAAGAAGTCTGCAATTCCCTATGGGAACAAGTCACAGTATATCCTTTCAATAAGCAGAGAAAACTTAA | |
| ACAAGGACCTGCTGACTGCCGWGGAGTCCTATGCCAATGCGAAGGTGCACTTTGGCCACAAGCTGTCGAAATGCATTCCG | |
| GAGGAAGGGGTACTCACAGWGCTCGGACCTGACAAGGTTCCCCGAGATGTCACATGTGACCTTGTTCTAGGGTGTGATGG | |
| AGCCTATTCAACTGTCAGAGCCCACCTCATGAAGAAGCCCCGCTTTGATTACACTCAGCAATATATCCCTCATGGATACA | |
| TGGAGTTGACAATTCCACCTAAGAATGGGGAGTACGCCATGGAACCTAACTGTCTTCACATTTGGCCTAGAAATGCCTAT | |
| ATGATGATCGCCCTTCCGAACATGGACAAATCTTTCACATGCACCTTGNTCATGCCCTTTGAGGAGTTTGAAAGACTTCC | |
| AACGCGCAGCGATGTGCTGGACTTCTTCCAGAAGAACTTTNCAGATCCTATCCCTCTGATGGGAGAGCAAGCCCTCATGA | |
| GAGA | |
| Clone 0018-Contig | |
| CCCTTTTNNCCCCAATTGGGCCGTTAGGCGTTTCCGGTGGAAAGGTCCTANNTTAAGCAGANGCCNCTTCGGGGCTAACC | |
| TANAAGNNCCCCNCTTGGNTTNCCNGGCCTTNTCGGAAATTTNATTTCGGACCNCCCCTTTAAGGGAGNCCCCAAGCTTG | |
| GGTCCCAAACCTCGGNTTCCCCTAGTTCCGNGCGGCCAATTGTGCTGAAAGCGRCTGCAMAGTGAAGCCCTCTGTTACCT | |
| GTGTCGATCAAGACCTCAAWCCCCAGAGGAACTTCGTCATCAACATGACTTGCAGGTTTTGCTGGCAGCTTCCTGAAACA | |
| GACTACGAGTGTTCAAATTCCACCACCTGCATGACCGTGGCTTGCCCTCGGCAGCGCTATTTCGCCAACTGCACCGTGCG | |
| TGACCACATTCACTGCCTGGGCAACCGGACTTTCCCTAAGCTGCTGTACTGCAACTGGACAGGTGGCTACAAGTGGTCGA | |
| CAGCCCTGGCTCTCAGCATCACCCTTGGGGGGTTTGGAGCCGATCGCTTCTACCTGGGCCAGTGGCGAGAAGGCCTCGGC | |
| AAGCTCTTCAGCTTTGGCGGCCTGGGAATATGGACCCTAATCGATGTCTTGCTGATTGGAGTTGGCTATGTGGGACCAGC | |
| GGATGGCTCTTTGTACATTTAGCCGAGGTTATGTGCTTCAGAGAGCAGTGTAGAGTCCTGTGTGTGGAGATGGATGCGGT | |
| GTGAGAGGGGGATTTATGTTTTTAATGTACAGCATCTGTACTTGGCTTGCCTTGATGAAGGTAAAATAAAGAAATCGAAC | |
| MCTGAAAAAAAAAAAAAAAAAAAAAAA | |
| Clone 0019-T7 Sequence | |
| CCTGGTAGGAGCCGCAGTGTGTGGCCATGCCTCAGATAGGTTTGGGCGCAGAAGGGTGCTGACCTGGAGCTATCTTCTGG | |
| TGTCCGTGTCCGGCACAGYAGCTGCCTTCATGCCCACCTTCCCCCTCTACTGCCTGTTWSGTTTCCTGCTGGCCTCTGCA | |
| GTGGCAGGTGTCATGATGAACACAGCCAGTCTCTTGATGGAGTGGACATCAGCCCAGGGTAGCCCTTTGGTGATGACCTT | |
| GAACGCCTTGGGCTTCAGCTTTGGGCAGGTCCTGACAGGTTCTGTGGCCTATGGTGTGCGCAGCTGGAGGATGCTACAGC | |
| TGGCTGTCTCTGCCCCCTTCTTCCTCTTCTTTGTTTATTCATGGTGGCTACCAGAATCGGCACGCTGGCTCATCACGGTA | |
| GGCAAGCTGGACCAGGGCCTGCAGGAACTACAGAGGGTAGCTGCTGTCAACAGGAGGAAGGCAGAGGGAGACACGTTGAC | |
| CATGGAGGTGCTGCGGTCAGCTATGGAGGAGGAACCAAGCAGGGACAAAGCTGGTGCCAGTCTGGGCACTCTACTCCACA | |
| CACCTGGGCTGCGCCACCGAACCATCATCTCCATGCTGTGCTGGTTTGCCTTTGCCTTCACCTTCTACCGCCTGGCCCTT | |
| GACCTGCAAGCCCTAGGAAGCAATATCTTCCTGCTCCAGGCACTCATCGGGATTGTGGACTTCCCGGTGAAGACAGGCAG | |
| CCTGCTGCTGATCAGCCGCTTGGGCCGGCGCCTCTGCCAGGTCAGCTTCCTGGTGCTCCCCGGACTCTGCATCCTGTCCA | |
| ACATACTGGTGCCCCATGGGATGGGGGTCCTTCGCTCGGCCCTGGCTGTGCTGGGGCTGGGCTGCCTGGGGGGCGCCTTC | |
| ACCTGCATCACCATCTTTTCCAGTGAGCTCTTTCCCCACTGNGATCAGGGATGACTGCAGNGGGCC | |
| Clone 0019-BGH Sequence | |
| TTTTTTTTTTTTTTTTTTTTYTCTTTAAAAATTATTTATTTGKGTGTATCACCGGGGGCGGGTATGTGTCCATGCGGAGC | |
| TCACAGGACAACTTTGTGAAGTCTGTTCTCACATGAGTTTCAGAAATTTAGGCCCGGAGGCAGGTGTCTTTACCCGCCGT | |
| CGCCAGCCCTGCCCTTTCCTCCTCCTCCTCCTCTTCCTCCTCCTCCTCCTCTWCCTCCTCC | |
| Clone 0020-Contig | |
| GAAAGGGGACGGAGACCTTCAGCGTGGAATCTATATCCAAGAATGGAATCTGTCTGGAGATGGGCCCACAGCCTCAGGGC | |
| GTGCTGCGGGCCGACCTGTTCTCCCGGATGCGAGCTCTGGTGGCATCCATTCTGGACTTCATCGAGCTCTTCAACCAAGG | |
| CATGGACTTACCCGCCTTTGAGATGGATATCTACAGGAACTTGGGCAGTGTGGACTTCCCACGCACTGCGGATGGTGACC | |
| TGGCTGGCACTGTGCACCCTCAACTGCAGGACCATTACTTTGAGCCACTGAGGCCTGGTGAACCCATCTTCAAGCTTTTC | |
| AGCGGAGAAGACGTACTGTATGAGGGGGACTCCATTGTGTACCCTGTGTTCATTAATGAGGCTGCCTATTATGAGAAGCA | |
| CGTGGCATTCCTGAAGTCTGAGAAGATCAGGGTCACAGTGCCTGCCCTGCTGAGGTTGACCCCCCGCTCCACCCAGACTC | |
| CCTAACCCAAATCATACCTGGCCTGATCTCAGTTTTCCTATCTGAACAATGGGTCCCAAGCCCAGGGTCCCCTATCTCAC | |
| TCTGGACACCATATCCCTTTCCAGACAACTGATCTCATTATATAAAATAGACTGTGCCCCAAAAAAAAAAAAAAA | |
| Clone 0022-T7 Sequence | |
| AGCGGACGTTTGTGCCGGGACATGGCCGCTGCGGATGCCGGGTCCTTGAGCTGAGCGCTTGCTGCCGGAGCCAACCTCTG | |
| CCGTCAACCGTCCGCGCGGGCTGGGCCCAGGCGCCGGGACGGCCAAGATCCGCCGAGGAAGCTGAGGCAGCTATAGAACG | |
| CCGCCGCGGCGGGCGCATGGCGTCCATCTTGCTCAGGAGCTGCCGGGGCCGGGGGCCTGCCCGCCTCGCGCCACCTCGGG | |
| CCGCCTCCCCGCGGGGTAGTCTGAGGGATCGAGCTTGTCTCAGCTGTACCAGGACCCTGGGGTTGACGAGCCGTGAGAGT | |
| GTTCTGTCTCGTTGCTGTACTCCAGCCCACCCTGTGTACCTCTGCTTCAAAGGTGAGCCCCTCAGCTGTTGGACTCAGAG | |
| GCCTGAGTGCCAGGGCACCGCAGCAAGAACAACATGGACACCTGCCTCTGCAAGGCTGGTGGTTACGGGACCTCAGTACC | |
| TTCCTGTGCGCGGCTGGCACTCATCATCTYCGCTAGGAGAGGACTCTGTGATAGAGAAGTCCCTTAAGTCCTTAAAAGAC | |
| AAGAATAAGAAGCTGGAGGAGGGCGGCCCCGTGTACAGCCCGGCCGCGCAGGTGGCTGGTGAGGAAATCCCTNGGGCAGA | |
| AGGTACTGGATGAGCTGAGGCACTACTACCATGGCTTTCCGCCTGCTCTGGGATNGACACCAAAGANCGCTTG | |
| Clone 0022-BGH Sequence | |
| TTTTTTTTTTTTTTTTTTTTTTTTTTTTTTTTTAATTATGGAAGTTTCTGATTTATTCCAGACAAAATATTAGATTTGCC | |
| ACTAAGAATCACCTCAAAGCAATCACTCTTGNGTGTCATGCCCCTGNGGCTGGCACGGCATGATGAAGGCAGACCTGNGG | |
| GCCAGAAGCTGGCAGCTCTAATTCTTCACTTCTGCGGCCTCTTTCTCAGCCTTCTCCTTGGCCTTCTCTTTCTCTTCTAT | |
| CTTTTCTTCTTTCTCTAGNGNGGCCACAATTTCAGCTACCTGGGNGGNAGAGATCTGAACATCTTCTTTGTTCACCAAGT | |
| CGATTACCTTGACAAGGTCATCGATGTTGATATTGCCGNCCTTATTATCATCCAGGGCTGAGGTCAAACTGATGAGCTTG | |
| AGCTCTGGAATGNGCTTGATTTGCTTCATGGCGCTGATGAGCTCAGTGATGCTGATGACACTCTCCCCTGTWGGAGNGCT | |
| CTGGCTGGGGCCCAGCTTGCCATCCTGWTGAGAGGWCTCCAGCTGTGTGATCAGGCCATCGATCTGCCCGATCATCTGCT | |
| GCACTCGCTTTGACAGCCTCTTGCTCGCTGCARAWTCTTCTATGNACTTTCTCCTCACCCGTCTNTTGAAAGKTCCTTCT | |
| TTGAWCTCCTGTAAGTCCTCACNTGTAGGCCTGGACGCCCTCTTTTGAGMRSRSNTCCAGGCTTCCTCCTTTCTCCCTTG | |
| GTCAGGGACTTCTTCTGCNCTGAGCTTGGAACAGGCGTACTGAGGATGTNAA | |
| Clone 0023-T7 Sequence | |
| ATGGACCGCTTCGTGTGGACCAGCGGCCTCCTGGAGATCAACCAGACCCTGGTTATCCAGCAGCGCGGGGTGCGCGTCTA | |
| CGACGGCGAGGAGAAGATAAAATTTGATGCCGGGACTCTTCTTCTTAGTACACACCGGCTGATTTGGAGAGACCAGAAGA | |
| ATAATGAGTGCTGCATGGCCATTCCCCTGTCTCAGATTGTGTTCATCGAGGAGCAGGCAGCTGGAATCGGGAAGAGCGCC | |
| AAAATCGTGGTTCACCTGCACCCAGCTCCTTCTAACAAAGAGCCCGGTCCGTTCCAGAGCAGTAAGAACTCCTACATCAG | |
| ACTCTCCTTCAAAGAACATGGCCAGATTGAGTTTTACAGGCGTTTATCAGAAGAAATGACACAGAGAAGATGGGAGACTG | |
| TGCCAGTTTCCCAGTCACTACAAACAAATAAAGGACCCCAGCCAGGAAGAGTAAGAGCTGTTGGAATTGTAGGTATTGAA | |
| AGGAAACTTGAAGAAAAAAGAAAAGAAACCGATAAAAACATTTCTGAGGCCTTTGAAGACCTGAGCAAGCTGATGATTAA | |
| GGCTAAGGAAATGGTGGAGTTATCCAAATCAATCGCTAATAAGATTAAAGAGAAGCAAGGCGATGTCACAGAGGATGAGA | |
| CCATCAGGTTTAAGTCGTACCTGCTGAGTATGGGAATAGCCAACCCAGTCACCAGAGAAACCTATGGCTCCGGCACACAG | |
| TACCACATGCAGCTGGCCAAACAGGCTGGCTGGAATATTGCAGGCACCTTTGGAGGAGCGTGGGGGAATAATGNCCCTCA | |
| CACAGGNG | |
| Clone 0024-T7 Sequence | |
| AAAGTGGGGATCCTGGWGACAGTTCTGGTGCAGAGTTCCAGTACCTCTACATCCATCATTGTCAGCATGGTCTCCTCCGG | |
| CTTGTTGGAGGTGAGCTCCGCCATTCCGATCATCATGGGCTCCAACATCGGAACCTCTGTCACCAACACCATTGTGGCCC | |
| TGATGCAGGCAGGGGACAGGACTGACTTCAGGCGGGCTTTTCCAGGGGCGACCGTGCATGACTGTTTTAACTGGCTGTCT | |
| GTTCTGGTCCTACTGCCCCTGGAGGCTGCCACGGGCTACCTACACCATGTCACCGGGCTTGAGGTTGCTTCCTTCAACAT | |
| CCGAGGTGGCCGTGATGCCCCCGACCTTCTCAAAGTCATCACAGAGCCCTTCACAAGACTCATCATCCAGCTGGACAAGT | |
| CTGTGATCACCAGCATTGCCGTGGGGGATGAGTCCCTGAGGAATCACAGTCTCATTCGGATTTGGTGTCACCCAGACACA | |
| ACAGAGGCTTCCACTTCTATGTCCAGGGTAGAGGCCATCGGCAGCCTTGCAAACACCACCATGGAGAAATGCAACCATAT | |
| CTTCGTGGATACGGGGCTGCCCGACCTGGCCCGTGGGGCTCATTCTGCTGGCGGGCTCCCTGGTGGTTCTCTGCACTTST | |
| CTTATCCTCCTTGNCAACATGCTCAACTCCCTGCTCAAGGGCCAAGTGGCCAATGTCATCCAGAAGGTCATCAACACAGA | |
| CT | |
| Clone 0025-T7 Sequence | |
| AGGGCGCCAGCTGAAGACGCGGGACTTAAAGCGCGTAGCCAGAACCCAGGCACCAGTGTGTCCATTGTCCAGAACTCATC | |
| TGAAAAACTGCCACAGGAATTGCTTCTCTGCTCCAGGCTGGTCACTGAACAGGTTGCTCCAGGACCTGCAGAATGGGGGC | |
| AGGCTGTGTCAAAGTCACCAAGTATTTCCTCTTCCTCTTCAACTTGCTGTTCTTTATCCTGGGTGCTGAGATCCTGGGCT | |
| TCGGGGWGTGGATTCTTCCACACAAGAACAGCTTCATTTCCGTCCTACAAACCTCATCCAGCTCGCTGCAGGTGGGGGCT | |
| TACGTCTTCATCGGWGTGGGCGCCATCACCATAGTGATGGGCTTCCTGGGCTGTATCGGWGCTGTCAATGAGGTCCGCTG | |
| CTTGCTGGGTCTGTACTTTGTCTTCCTTCTGCTGATCCTCATCGCACAGGTGACCGTAGGGGTCCTCTTCTACTTCAACG | |
| CTGACAAGCTGAAGAAGGAGATGGGGAACACAGTGATGGACATCATTCGCAACTACACTGCCAATGCCACCAGTAGCCGC | |
| GAGGAGGCCTGGGACTACGTGCAGGCGCACGTCAAGTGCTGTGGCTGNGTCAGCCACTACAACTGNACAGAGAACGAGGA | |
| CCTCATGGGCTTTACCAAGACCACTTACCCATGCTCCTGCGAGAAGGATCAAGGNAAGAGGACAACCAGCTCATTGTGAA | |
| GAAAGGATTCTTGCGAGGCTGATAACAGCACCTGTGAGCGAAAACAACCCCTGAGGATT | |
| Clone 0026-T7 Sequence | |
| AGGAGCCCTCGAAAGCGACATGGCGGTTCTCTTAAAGCTGGGCGTTCTCTGCAGTGGCCAAGGAGCTCGAGCTCTCCTAC | |
| TCCGAAGCCGGGTGGTCAGACCCGCTTATGTGTCAGCATTTCTCCAGGACCAGCCTACCCAAGGACGGWGTGGTACCCAG | |
| CACATTCACCTGTCACCAAGCCACCACTCTGGTTYCAAGGCTGCATCTCTCCACTGGACCAGTGAGAGGGWWGTCAGTGT | |
| TCTGCTCTTGGGGCTGATCCCTGCTGGGTACTTGAATCCCTGCTCTGTGGTGGACTACTCTCTGGCTTCAGCCCTCACCC | |
| TGCACAGTCACTGGGGCCTTGGACAAGTGGTTACCGACTACGTTCATGGGGACACCCTGCCGAAGGCTGCCAGGGCAGGC | |
| CTCTTGGCACTCTCAGCTTTGACCTTTGCTGGGCTTTGCTACTTCAATTACCACGATGTCGGCATCTGCAGAGCGGTTGC | |
| CATGCTGTGGAAGCTCTGACCTGGGTGCAGCACTTTGATTGTGTGCCTCCTTGCCTCTGCTTTACCAATGCCGTTCACCT | |
| CGCAGTGAGGGGGGATGAAGGATAAGCCCATTGGTGGGCAGAATGTCTTCTAATTACATGGGTATTTTCAGAATTATTTG | |
| NTGAGGAAGAGGNTT | |
| Clone 0028-BGH Sequence | |
| TTTTTTTTTTTTTTTTTTCTTTTTTAAGATAATGGTCTTTTAATTGAGTTATTGAGATGAAGAGACAGTGAAGCCCTGTT | |
| TGCTACTTACATGAAAAGAAGATTTTAAAAAACAATCACTGCACAAAATACAAAGGGGCAGGGTATGCTGWGGCATTGAA | |
| TTCCTCCCCACGTTTTTTCTTGACTTCTCAAGAACAAATTAAAGTCTCCACAGCAAATTTGTTCTCAAAATGCCGAATGG | |
| TGAAACAGTTACTGGCTTCCCGCTTCTGAATACCTCTAATTGTTTCCCGGCGCTGCAGTTTGTAGGTCTCCTTGTCGTGA | |
| CACAGTCGGTAGATGAAGAAGCCCAGGTTGTCCACGTTCTCGATGCGGTCGGTGATCACCATGTGCTCATGGATAAGGTA | |
| GGACTGAGGCAGGTAGGTCCCGGCCTTAATGTTAATAAGGAGCTCCAGCAGGTTTTTGGGCGGCATAACGATGGAAGTGT | |
| TCAGAGGATCACGTAGCACTTGTCCAGGTTAAGGT | |
| Clone 0029-T7 Sequence | |
| AAAGAGCGGCTGCTGTCGGAAGCACCGGGCGAGCTATCTGTTACAGTCCGGCCCGGGGATGGCTCGGGACGCGGAGCTGG | |
| CGCGCAGTAGCGGGTGGCCGTGGCGGWGGCTGCCGGCGCTGCTGCTGCTGCAGCTGCTGCGGTGGAGGTGCGCCCTGTGC | |
| GCGCTCCCCTTCACCAGCAGTCGGCACCCAGGCTTTGCGGACCTGCTGTCGGAGCAGCAGCTGTTGGAGGTGCAGGACTT | |
| GACCCTGTCTTTGCTGCAGGGCGGAGGTCTAGGGCCGCTGTCACTGCTACCTCCGGACCTGCCGGATCTGGAGCCTGAGT | |
| GCCGGGAGCTGCTGATGGACTTCGCCAATAGCAGCGCCGAGCTGACCGCCTGTATGGTGCGCAGCGCTCGGCCCGTGCGC | |
| CTCTGCCAGACCTGCTACCCGCTCTTCCAACAGGTCGCAATCAAGATGGACAACATCAGCCCGAAACATCGGGAATACCT | |
| CCGAGGGCCCGCGCTGAGGCGGAAGTCTCCTGACGGCAGACAGAATGCAGATAGTTCTCATGGTCTCTGAGTTTTTCAAC | |
| AGCACGTGGCAGGAGGCGAACTGCGCAAATTGCCTAACAAACAATGGTGAGGATTTGTCAAACAACACAGAGGACTTCCT | |
| CAGTCTGTTTAACAAGACTTTGGCCTGCTTTGAGCATAACCTGCAGGGGCACACATACAGNCTCCTTCCACCAAAAAATT | |
| ACTCCGAAGTGTGCAGAAACTTGTAAAGAGGCATATAAAAACCTGAGCCTNCTGTACAGTCAAATGNA | |
| Clone 0029-BGH Sequence | |
| TTTTTTTTTTTTTTTTTTTTTTTTTTTTTTTTTTCCTTGTTATTTCCTTTATTGGAAAGCATAAGGAAAAAACAGGTTTT | |
| CTTGDGCACACACATAACCCTATGKGCCTMAGGATTCAGAACTATGTACATTTTTTTAATATGACCACAAGATGAAATTC | |
| TTTGGCACATTTTCAAATATATTTCTAATGCAACCTCTAGAGAGCCAGACCCTGATCAGGAACAGAGATGGCTGGCTTGT | |
| TAAAGGGCTCTCCAGCTTCTTAGCCAAAAGCAGGGGTTTGTACACACAGTACTGAAAGGNACCCGAGGAAGTCGCTACTC | |
| ACAGGTTTAAATATGTCACTTCACTCACTTTGCACATGTAAATAAGNTTTACATGTACTGATGAAGATGGNTTCCAATGA | |
| CCCTNAACCATGNGCTTCAAATCAAGACAGGAACAATGACAGCNCAATGAACCCCGGCACATNTAGGGGATCACAGCGNC | |
| GNCTGATTGTCACATACCCGGGGTGACACACTCTGGGACTAAGACTAGCCTGCTNTCACACTCTGCANATGTGGNAAACA | |
| TACAAAAAATACCCAAACACTCCTGCCTTCCTGTAGGGCAAANACAGGTTTTNAAGG | |
| Clone 0030-Contig | |
| AAAGGCGCGGAGCCTGCTGCTGCCATGGTGGCTGGTGGCTGGGTACACTATGTCTAGACTGGGGGCCCTGGGCGGCTCCC | |
| GCGCCGGGTTCGGACTGTTACTTGGTACTGCCGCCGGCCTTGGATTCCTGTGCGTCCTTTACAGCCACCGATGGAAACGG | |
| ACCCAGCGCCATGGCCGGAGTCACAGTCTGCCCAACTCCCTGGACTATGCGCAGGCTTCAGAGCGTGGACGCCAGGTGAC | |
| ACAGTTTCGGGCTATCCCAGGTGAAGCTGGAGATGCTGCCATACTGCCCAGCCTCTCACAGGAAGGGCAGGAGAAGGTCC | |
| TGGACCGCCTGGACTTTGTGCTGACCAGTCTTATGGCGCTGCGGCGCGAGGTGGAGGAGCTTCAGAGAAGCCTGCAAGGA | |
| CTAGCTGGGGAGATTGTCGGGGAGGTCCGCTCTCATATAGAAGAGAACCAGAGAGTGGCCCGGCGGCGCAGGTTCCCTTT | |
| TGCCAGAGAGAGGAGTGACTCCACGGGCTCCAGCTCTGTCTACTTCACCGCCTCCTCAGGGGCCGCACTCACAGACGCCC | |
| AGAGCGAGGGAGGCTATACAACAGCCAACGCCGAGTCTGATTACGAGCGCGACTCCCACAAGGAGAGTGGAGATGCTGAG | |
| GACGAAGTGAGCTGCGAGACCGTGAGGATGGGGAGGAAGGACTCTCTGGACCTGGATGTGGAGGCGGCGTCCAGTCCGTC | |
| Clone 0031-T7 Sequence | |
| AGGCTTGCCGTCCAGGCCATGCGGCTCGWGGTCTGCATCGWGGCGTTTCCCATGTTTCTGCTGAACCTTCTAGGCATGTG | |
| GAGCTGGGTATGCAAAAAGTGCTTTCCCTACTTCCTGAAGCGGTTCGCCATGATATACAATTGGAAGATGGCGAGCCTAA | |
| AGCGGGAGCTCTTCAGCAATCTGCAGGAGTTCGCCGGCCCCTCGGGGAAGCTAACTCTGCTGGAGGTGGGCTGCGGCACC | |
| GGGGCCAACTTCAAGTTCTATCCCCCCGGGWGCAGGGTCACTTGTATCGACCCYAACCCCAACTTTGAGAAGTTCTTGTT | |
| CAAGAGCGTCGCAGAGAACCGGCAGCTGCAGTTCGAGCGCTTCGTGGTGGCAGCCGGGGAGGACATGCACCAGGTGACCG | |
| ATGGCTCTGTGGACGTCGTGGTCTGCACCCTGGTGCTGTGCTCGGTGAAGAACCAGGAGAAGATTCTGCGCGAGGTGTGC | |
| CGAGTGCTGAAGCCGGGAGGGGCTTTTTACTTCATAGACCATGTGGCAGATGAACGGTCTACCTCGAATTACTTCTGGCA | |
| GCAGGTCCTGGCTCGTGTGTGGTTCCTTGCTTTTGACGGATGCAATCTGACCAGAGAGAGCTGGAAGGCCATAGAGCAGG | |
| CCAACTTCTCCAAGCTGAACCTGCAGCACATNCAGCCCCCGCTCCCCTTGACCTTCGTGCGGNCCCACATCTATGGCTAT | |
| GCTGTGAAATAGAGT | |
| Clone 0031-BGH Sequence | |
| TTTTTTTTTTTTTTTTTTTTTTTTTTTTTTTTTTTTTTGGCAAATATAGAAACCATTTATCAAATGAATATAAATGTATT | |
| GATCAACATTTAAAATATAACTTCTGCAAAATCATCTTGAAAAATATACATTTGTTTAGATCCATACATACAAATGCAGC | |
| TCAAACCCTTGGGCCCACCCAGACTTGCTCTCTGTATGAACACAATGATATCCATGGTTTTGTTTCAGGACCAGKGGAAT | |
| TTTTCTTCTTCTTCAATACAGGGTTTATTTGTGTAGCCCTGGCGGKCCTGGAACTCCATCTGTAGATCAGGCTGTACTCA | |
| CAGAGATCCACCTACCTCTGCCACCTGAGAGCTGGGATTGAGGCTCTGCCACCAACCACTCAGGACCAGTGATATTTGAC | |
| CAGAAGAATCCCTCCCCCCCCGSACCCCGAGTCCTTGGTAACTACTCTCGGGCATTACTTTTAGGGKCCCCTACATACTG | |
| GACCCATTTCCCTAACTATAGNGGCCTTCTACTGCCTACGGTAATTAT | |
| Clone 0032-Contig | |
| AAAGAAGGAACTAAACATGGGCCAGCGATGCTCTGACACCAGAGGAATTGCTTTCGAAGACGTCAGAGTGCCTAAGGAAA | |
| ATGTGTTAATCGGTGAAGGAGCACKTTTCAAGATCGCAATGGGTGCTTTTGATAGAACCAGACCTACAGTCGCAGCTGGC | |
| GCTGTCGGGCTAGCCCAGAGAGCTCTGGACGAAGCCACGAAGTATGCCCTGGATAGGAAGACATTTGCAAAGCTGCTAGT | |
| GGAGCACCAAGGAGTTTCATTTCTGCTCGCAGAAATGGCGATGAAGGTTGAACTCGCTAGGCTCAGTTACCAGAGAGCAG | |
| CCTGGGAGGTTGACTCCGGTCGCCGGAACACTTACTATGCCTCGATTGCAAAGGCCTTTGCTGGAGACATTGCCAATCAG | |
| CTAGCCACTGACGCCGTGCAGATTTTCGGAGGCTATGGATTCAACACAGAGTACCCTGTGGAGAAGCTGATGAGGGACGC | |
| CAAGATCTATCAGATTTATGAAGGTACTGCACAAATTCAGAGGCTGATCATAGCTCGTGAGCACATTGAAAAGTATAAAA | |
| ATTAACAGGAATTACTATTGAACGATGCATCACCCTCGTGTAACTAACCTCCAAGCACTGTTCCTGCTTCAGGGGAAAAG | |
| GGCTTTACTGTCTTCCCAAGGAAATGAGATAAAAGACGAGTTTGGATCTATGCGGCGGATTCCCATGGCGGAGGAACCTG | |
| TCTTCAGCTCTATGGTGACCCTTTCTAGATAGGTTTGGCTTTTGGACAATGATTGGTCCTTAGCCCCGAATTGTGTTAGT | |
| TTGCTCTTTGATCACTTAAAATGGAAAAACACCCTGGACTTTTAATGTTCATTCAAGTGACAGGAAAGGCGGCTTGTCAA | |
| GGAAGAACTCATGATTCTAACATAAACACTGAAAATTTGTGGTAGATTGGACACGTCAGACTGTGACATAGCAGCATTTC | |
| TGTGCTGAACTGTTAATTTTATAATTTTGATTATATTTGCTTTGTTTTGCACAAAAGAGTAAAAAGTTTATATTCATATT | |
| CTCCCATTATAAAACTAAAACTTTTCTGGGAATCTTAGTACTGAACGAGAATTTTATTTGTCTTGTTTAAAATAATTCAA | |
| TAAAGCTAACCTTAACTTAAAAAAAAAAAAAAAAAAAAAAAAAA | |
| Clone 0033-Contig | |
| AGCGTCGCCATCCGCCACCATGGTGAACTTCACAGTAGATCAGATCCGTGCCATCATGGACAAGAAAGCCAACATCGATA | |
| AGGGTGAGGTGTCTGCCCGCCAGGAGCTCAAGGCACGTGCCCGCTACCTGGCCGAAAAGTATGAGTGGGACGTTGCTGAA | |
| GCCCGCAAGATCTGGTGCTTTGGCCCTGATGGCACTGGCCCCAACATTCTCACCGACATCACCAAGGGTCTGCAGTACCT | |
| GAATGAGATCAAGGACAGTGTGGTGGCTGGCTTCCAGTGGGCTACTAAGGAGGGCGCTCTCTGTGAGGAAAACATGCGTG | |
| GTGTGCGGTTTGATGTTCATGATGTGACCCTGCATGCTGATGCCATTCACCGGGGAGGTGGCCAGATCATCCCCACAGCA | |
| CGCCGCTGCCTGTATGCCAGTGTGCTGACCGCACAGCCCCGCCTCATGGAGCCTATCTATCTGGTGGAGATCCAGTGTCC | |
| TGAGCAAGTGGTGGGTGGCATCTACGGTCTCCTGAACAGGAAGCGTGGCCATGTGTTTGAGGAGTCCCAGGTGGCTGGTA | |
| CCCCCATGTTTGTGGTCAACGCATACCTGCCTGTCAATGAGTCCTTTGGCTTCACCGCTGATCTGCGATCCAACACCGGC | |
| GGCCAGGCCTTCCCCCAGTGCGTGTTTGACCACTGCCAGATCCTGCCTGGGGATCCTTTTGACAACAGCAGCCGCCCCAG | |
| CCAAGTGGTAGNTGAGACGCGCAAGCGCAAGGGCCTGAAAGAGGGCATCCCAGCGCTGGACAACTTCCTGGACAAACTGT | |
| AGGCAGCNTGATACTGCCACATGTTGCACAGTGCCCACCCATCAGAAGACCCCTTGAGACTGTCCCACAGTGCTCCTNTA | |
| GAGGCTGCTGGGGCCACCCTGACATCACTCAGCACTCACTTGCTACCAATTCTATTTATTTCGGAATTMCAAGATAGCGG | |
| GAATCTCTCTGCAGGCTGGACTGGCAGGCTGTGGGGTGGGCGGGACMCGGCTCTTAMCATTTTCARAGGGAAACGCGCAG | |
| ATGTCCAAAAGTYTAAATAAATGCATTCAGAGGTTTTTGGGGTCCATGGCCAAGTGGAGTTCCCCCARAGGGGGAGGTGG | |
| GGTAAGTGCCTCCAGGAAGGCAGGCAGCCTGCCTTAGMCTTGCAGCCCGCCTGTGGGAATGAAYCATTGGAGTAATAAAC | |
| TMCAGTGGTTGATCCAAAAAAAAAAAAAAAAAAAAAAAAAAAAAAAAAAAAAAAAAA | |
| Clone 0034-T7 Sequence | |
| AAAGGCCAGCTCCTGCTCTGCTCCTCTCCCGCCTGCCGCCGCGCTGCACGCCTCGAGCACTCCCTCGGCCCCGGCGGGGA | |
| CCGGGGACCCCGCAGCTACCGCCATGCTGCCAGTGCTCTACACCGGCCTGGCGGGGCTCCTGCTGCTGCCTCTGCTGCTC | |
| ACCTGCTGCTGCCCCTACCTCCTCCAAGATGTGCGGTACTTCCTGCGGCTGGCCAACATGGCCCGGCGGGTGCGCAGCTA | |
| CCGGCAGCGGCGACCCGTGCGTACCATCCTGCGGGCCTTCCTGGAACAAGCGCGCAAGACCCCACACAAGCCCTTCCTGC | |
| TGTTCCGAGACGAGACGCTCACCTACGCCCAGGWGGACCGGCGCAGCAACCAAGTGGCGCGGGCGCTGCACGATCAACTG | |
| GGCCTACGACAGGGGGATTGCGTAGCCCTCTTCATGGGCAATGAGCCGGCCTACGTGTGGATCTGGCTGGGACTGCTCAA | |
| ACTGGGCTGTCCCATGGCGTGCCTCAACTACAACATTCGTGCCAAGTCTCTGCTGCACTGCTTTCAATGCTGCGGGGCGA | |
| AGGTGCTGCTGGCCTCCCCAGATCTACAAGAAGCTGTGGAGGAGGTTCTTCCAACCCTGAAAAAGGATGCCGTGTCCGTC | |
| TTTTACGTAAGAAGAACTTCTAACACAAATGGTGTGGACACAATACTGGACAAAGTAGACGGAGTGTCGGCGGAACCCAC | |
| CCCGGAGTC | |
| Clone 0034-BGH Sequence | |
| CTTTTTTTTTTTTTTTTTTTTAGTGTTAATATAGTTTATTATGTCTTTAAAAAAATAAGGCCCTCTCTCCAAGAAGCTTA | |
| GTTTGCAAGGACAAATGGCAGGTGCACATTGAAAAATAATTGTTTCTAAATCTTTTTACTTGCAAAGGTTCAGGTGTAAT | |
| TTAAAAAAAAAACAAACAAACTATCCTTTTAATGAATAATTTCCTAAAAATAAAATCGCACCTTATAGCCTTCAATCAAG | |
| TTAAAGTTGGATTCTACGTATGAAGTGGCTCTGCGAGGTCTATCGAGTTTCTTTCTGGAAATGTCATGAGCTAAACCACC | |
| AGGGAATATTCAGAGCTTCAGAGTTTTATCAATTATGGCATTATAAATGTTCTCAGTCATGGGCACAAATGTTTTCTCTG | |
| CATCATCCATGAAATACAAGGTATCTTTGATGACTGTGGGATTGAAGCCCTCTTCCATCAGGGTCACTTTGCGGTGTTTA | |
| AAAGTCCCAGTGATCTCAATGGTATCTTGTATCCTCAGGAACCGAGGCCTCGCGTAACTGGGCAGGTACTCCGCGATGTG | |
| TTGAAAGAGTTTCTTTCCATTGAACTCGTAGTTTTCTTTGATCTTGAGGGAGGCCATCCCAATTCGACCCTCATGACCTG | |
| GCACAGGCACGCCATACACATTCACTTCTTCAACAAAATCTACCAGTCCCACGATGTCAGCGACCTTCTGNGGTAGCTAC | |
| GNTCTCTTCTTTTCACCGGAAAGTATCTNCAACCCTG | |
| Clone 0036-Contig | |
| AAAAGGTGGAATCTATATCCAAGAATGGAATCTGTCTGGAGATGGGCCCRCAGCCTCAGGGCGTGCTGCGGGCCGACCTG | |
| TTCTCCCGGATGCGAGCTCTGGTGGCATCCATTCTGGACTTCATCGAGCTCTTCAACCAAGGCATGGACTTACCCGCCTT | |
| TGAGATGGATATCTACAGGAACTTGGGCAGTGTGGACTTCCCACGCACTGCGGATGGTGACCTGGCTGGCWCTGTGCACC | |
| CTCAACTGCAGGACCATGACTTTGAGCCACTGAGGCCTGGTGAACCCATCTTCAAGCTTTTCAGCGGAGAAGACGTACTG | |
| TATGAGGGGGACTCCATTGTGTACCCTGTGTTCATTAATGAGGCTGCCTATTATGAGAAGCACGTGGCWTTCCTGAAGTC | |
| TGAGAAGATCAGGGTCACAGTGCCTGCCCTGCTGAGGTTGACCCCCCGCTCCACCCAGACTCCCTAACCCAAATCATACC | |
| TGGCCTGATCTCAGTTTTCCTATCTGAACAATGGGTCCCAAGCCCAGGGTCCCCTATCTCACTCTGGACACCATATCCCT | |
| TTCCAGACAACTGATCTCATTATATAAAATAGACTGTGCCCCAAAAAAAAAAAAAAAAAAAAAA | |
| Clone 0037-T7 Sequence | |
| AGCTGCATCGTGGCGTTTCCCATGTTTCTGCTGAACCTTCTAGGCATGTGGAGCTGGGTATGCAAAAAGTGCTTTCCCTA | |
| CTTCCTGAAGCGGTTCGCCATGATATACAATTGGAAGATGGCGAGCCTAAAGCGGGAGCTCTTCAGCAATCTGCAGGAGT | |
| TCGCCGGCCCCTCGGGGAAGCTAACTCTGCTGGAGGTGGGCTGCGGCACCGGGGCCAACTTCAAGTTCTATCCCCCCGGG | |
| TGCAGGGTCACTTGTATCGACCCCAACCCCAACTTTGAGAAGTTCTTGTTCAAGAGCGTCGCAGAGAACCGGCAGCTGCA | |
| GTTCGAGCGCTTCGTGGTGGCAGCCGGGGAGGACATGCACCAGGTGACCGATGGCTCTGTGGACGTGGTGGTCTGCACCC | |
| TGGTGCTGTGCTCGGTGAAGAACCAGGAGAAGATTCTGCGCGAGGTGTGCCGAGTGCTGAAGCCGGGAGGGGCTTTTTAC | |
| TTCATAGACCATGTGGCAGATGAACGGTCTACCTGGAATTACTTCTGGCAGCAGGTCCTGGCTCGTGTGTGGTTCCTTGC | |
| TTTTGACGGATGCAATCTGACCAGAGAGAGCTGGAAGGCCATAGACCAGGCCAACTTCTCCAAGCTGAACCTGCAGCACA | |
| TNCAGGCCCCGCTCCCCTTGACCTTGGTGCGGGCCCACATCTATGGCTATGCTGNGAA | |
| Clone 0037-BGH Sequence | |
| TTTTTTTTTTTTTTTTTTTTTTTGGCAAATATAGAAACCATTTATCAAATGAATATAAATGTATTGATCAACATTTAAAA | |
| TATAACTTCTGCAAAATCATCTTGAAAAATATACATTTGTTTAGATCCATACATACAAATGCAGCTGAAACCCTTGGGCC | |
| CACCCAGACTTGCTCTCTGTATGAACACAATGATATCCATGGTTTTGTTTCAGGACCAGTGGAATTTTTCTTCTTCTTCA | |
| ATACAGGGTTTATTTGTGTAGCCCTGGCGGTCCTGGAACTCCATCTGTAGATCAGGCTGTACTCACAGAGATCCACCTAC | |
| CTCTGCCACCTGAGAGCTGGGATTGAGGCTCTGCCACCAACCACTCAGGACCAGTGATATTTGACCAGAAGAAT | |
| Clone 0038-Contig | |
| AAAGGTGGAGCTGGGTGGTGTTTGGTGCGGTACGGCGGCCACTCACTTGCAGCAGAGCAGGTGCCATCCTGTGGAAGAAC | |
| CATGAAGCACTACGAGGTGGAGATTCGGGATGCAAAGACGAGGGAGAAGCTGTGCTTCCTGGACAAGGTAGAGCCTCAGG | |
| CCACCATTTCTGAAATCAAGACCCTTTTCACCAAGACACACCCGCAGTGGTATCCTGCCCGCCAGTCCCTCCGCCTGGAC | |
| CCCAAGGGGAAGTCCCTGAAAGATGAAGATGTCTTACAGAAGCTTCCTGTGGGCACCACAGCCACACTCTACTTCCGGGA | |
| CCTCGGGGCCCAGATCAGCTGGGTGACGGTCTTCCTGACGGAGTATGCCGGGCCCCTTTTCATCTACCTGCTCTTCTACT | |
| TCCGGGTACCCTTCATTTATGGCCGCAAATACGACTTTACGTCCAGTCGGCATACGGTGGTGCACCTCGCCTGCATGTGC | |
| CACTCGTTCCACTACATCAAGCGCCTGCTGGAGACTCTCTTCGTGCACCGATTCTCTCACGGAACCATGCCTTTGCGAAA | |
| CATCTTCAAAAACTGCACCTACTATTGGGGCTTTGCTGCATGGATGGCTTATTACATCAACCACCCTCTCTACACACCCC | |
| CTACCTATGGAGTTCAGCAGGTTAAGCTGGCACTGGCCGTTTTTGTGATCTGCCAGCTTGGGAACTTCTCCATCCACATG | |
| GCTCTTCGGGACCTTCGGCCTGCTGGGTCGAAAACCAGGAAGATCCCATACCCCACCAAGAACCCCTTCACCTGGCTGKT | |
| CCTGTTGCTGTCCTGTCCCAACTACACTTATGAGGTGGGCTCCTGGATTGGCTTTGCSATCTTGACTCAGTGTGTCCCAG | |
| TGGCCCTCTTCTCCCTGGTGGGCTTCACCCAGATGACTATCTGGGCCAAGGGCAAACACCGCAGCTACCTGAAGGAGTTC | |
| CGCGACTACCCGCCCCTGCGCATGCCCATTATTCCCTTCCTGCTCTGAGCAGCTCCTCACGGCTCTGCCCAGTAATACTC | |
| TCCACCCCTCACTGCCCCTGTCCTGATGTGTGGCTGGCCATGGCTCTCCAGCAGCAACAATAAAACCTGCTTACCCAAAA | |
| AAAAAAAAAAAAAAAA | |
| Clone 0039-T7 Sequence | |
| AAAGCGCTGGAGCTACAGCCGTTACTGCCGCCGCCGCCGCCGCCGCCGCCGAGGCGTTTGATCGTTGGCAATGTCAGGCT | |
| TTGATAACTTAAACAGCGGTTTCTACCAGACGAGTTACAGCATCGACGAGCAATCTCAGCAGTCCTATGACTATGGAGGA | |
| AGTGGAGGACCCTACAGCAAGCAGTATGCTGGCTGWGACTACTCGCAGCAAGGCCGATTTGTCCCTCCAGACATGATGCA | |
| GCCACAGCAGACATACACTCGGCAGATTTACCAGCCAACTCAGGCCTATCCTCCAACAACACCTCAGCCATTCTATGGAG | |
| ACAGCTTTGAGGAGGAGCCCCCTCTGTTAGAAGAGTTGGGTATCAATTTTGACCACATTTGGCAAAAAACACTAACGGWG | |
| CTACACCCCCCTGAGGGCAGCTGACGGCAGCATCATGAATGAKNACGGACTTGGCAGGGCCAGTGGTGTTCTGCCTTGCC | |
| TTTGGGGCCACACTGTTACTGGCTGGCAAAATCCAGTTTGGCTACGTATACGGGATCAGTGCGATTCGATGTTTAGGAAT | |
| GNTTTGNT | |
| Clone 0040-T7 Sequence | |
| CTCTCCTTTGCCCTGCGCACGGGTACACGGCACGGTGTGGACACTCACCTGTTCAGTGTGGAGTCGCCGCAAGAGCTGGC | |
| AGCCTGGACCCGACAGTTGGWGGATGGCTGTCATCGGSCTGCTGAAGCCGTACAAGAAGTGTCTACAGCCTGCACGTGGA | |
| ACGGCCGTCCCTGCAGCCTGTCTGTGCACATCGACAAGGGCTTCACCCTGTGGGCAGCTGAGCCTGGAGCAGCCCGAGCC | |
| ATGCTGCTCCGACAGCCCTTCGAGAAACTTCAGATGTCATCAGATGATGGCACGAGTCTCCTTTTCCTGGACTTTGGGGG | |
| TGCTGAAGGAGAGATCCAGCTGGACCTGCACTCGWGTCCCAAAACGATGGTCCTTTATCATCCACTCTTTCCTGTCCGCC | |
| AAGGTCACCCGCTTGGGGCTCTTGGCCTAGAAGCTGTCAGATGCACCAGCCCTGAAGAGGATCGTTCATCTTGTGGCCTG | |
| ACCTGTCCTTCTGCTGACTGCCTGCTCGTCTGGGGGCTGAGGGAAGGGAGAGGAGGGGAACTAGGGCCTCANAGACCAGC | |
| CTGAGGAAGCAGAACTCAGACCTGGGA | |
| Clone 0041-T7 Sequence | |
| AGGCTGTGCGAGCGGGCACGCGCGGACCCTKCACCGGGCCGCGGAGCCGCTATGGGCCCGCCAGGCCCCTCCCGTGGCCC | |
| GGCGCAAGGACCGGCTCCGAGCCAGGATTGAAATTCCAAGATGGATGATCAGGACCCTGGGGGCATTAGCCCCCTTCAGC | |
| AAATGGTGGCCTCAGGAGCCGGGGCTGTGGWCACCTCCCTCTTCATGACACCCCTGGATGTGGTGAAGGTCCGCCTTCAG | |
| TCTCAGAGACCCTCGGCAACCAGCGAATTGACAACTCCCTCCAGATTCTGGAGTCTCTCCTACACCAAATCATCCTCCGC | |
| TCTACAGTCCCCAGGGAAGTGCCTCCTATACTGCAATGGAGTCCTGGAGCCCCTGTACCTGTGCCCAAATGGTACCCGCT | |
| GTGCCACCTGGTTTCAGGACCCCACACGGTTCACTGGCACCTTGGATGCCTTTCWGAAGATTGTGCGGCATGAGGGCACT | |
| AGGACCCTGTGGAGCGGCCTCCCAGCCACCCDGGTGATGACCGYGCCAGCTACTGCTATCTACTTCACTCCTTACGACCA | |
| ACTCAAGGCCTTCCTGTGTGGTCAGTCCTTGACCTCTGACCTCTACGCACCCATGGYGGCTGNTGCCCCTCGCCCCGAAT | |
| GGGCACCGTGACAGTTGCAGCCCCTTGNAGCTCGTGCGGACCAAGCTGCAGGCTCAGCATGTGTCATACCGTGAGCTGGC | |
| TTNCTCTGTTAAGCTGCNG | |
| Clone 0041-BGH Sequence | |
| TTTTTTTTTTTTTTTTTTTTTTTTCACAGAAGACCATCATCTTTAGACAAGGAATGAATGGGGCCAAGATACTGCCTGGC | |
| CCAAGCTCCTCGGGCCAAGCTATAACAAACATGCGGATACAAAAAAGGCCGATCATCAGATTTGGTCTTGAATTCGGGAG | |
| GCGGGAGAGGGGCAATCCTGGGAAGGGAAACAGCAGGAATCCAGGTCTGAGGACAGCCTGGGWGACTGGACTGGGAGGGA | |
| AGGCACTTGGCTCAGTCTCCTGATCCCACCCGTGCAAGAGCCGGTTGCTCCCCGACTTTCAGCGGCCCANAGGCTGCTCC | |
| TGGTTGAGCCTCTGGAAGAAGCTTTTGCCAAACTCGTAAGTGCTGATCATGATGSCACAGGAGGGGTGCAGCCTTCATGA | |
| TCCTGGGGAGGAAAGCCTGCAAAGAGTCCCCTGGTGCCAGATTCAGCCCGG | |
| Clone 0042-T7 Sequence | |
| AAAGCTTCACTTCGCCCTCCAGCCGCGGWGGCTGCAGCGCAACTTCCAGATAGCGGAGTGGCCTCAGCTGCGAGCCGAGC | |
| GGWGGCGGCAGCCYYCCTCAGCACACCCCCAGATCACCTTTTCCCCGCGACTTCGCCATGGCTGACTGCTGWGTACCGGT | |
| ATGCCCACGGCCGATGTGTATCCCTCCACCCTATGCTGACCTCGGCAAAGCTGCCAGAGACATTTTCAACAAAGGATTTG | |
| GCTTWGGGCTGGTGAAGCTCGATCTGAAGACGAAGTCATGCACCCGTGTGGAATTTTCAACATCTGGCTCATCTAATACA | |
| GACACTGGTAAAGTTAGCGGGACCTTGGAGACCAACTACAAATGGTGWGAGTATGGTCTGACTTTCACAGAGAAGTGGAA | |
| CACCGATAACACTCTGGGGACAGAGATTGCAATTTGAAGACCAGATTTGTCAAGGWTWGAAACTTGACTTTTGACACCCA | |
| CCTTTTCACCGAACACAGGAAAGAAAAGTGGTAAAATCAAGTCTGCTTACAAGAGGGACTGTATAAACCTCGGCTGTCAT | |
| GTTGACTTTGATTTTGCTGGACCTGCCATCCATGGGTCAGCTGTCTTTGGTTACGAGGGCTGGCTTGCTGGGTACCAAAT | |
| GACCTTTGACAGTGCCAAGTCAAAGCTGACAAGGAGT | |
| Clone 0042-BGH Sequence | |
| TTTTTTTTTTTTTTTTCGGTTTTCATAAACGTCTATTTCATCATTGGTGGGTAGCACATTTAACAGTTAAATACATTTAA | |
| ATAATGTATAGGAGGCCGCACCACGGCAGCACTGATAACCATCCAACTAGGAACCAGCCAACAGTGACTGTCTAAATATT | |
| TAAAATACAGCTCTTGCTTCATCATCCTTTGATGTGATCACCTTCTGGGGGAAGGAAGGGGAGCCTGCTGGTCGCATGGA | |
| AATATATTAAGGCCAAATCTTCTGATATTCGTTCCCAGAGGTTTCTTTTAACTGGATTAAGCCTCCAATTCCAAGGCAAG | |
| CCCAAGTTTGTGGCCTCCAGCATTAAAGCTCTTCCCGTCTACCAGAGCAGACAGTGTAAGCTTCACACCAGGCCTCAGAG | |
| TCTGAGTATAGCCCACTCCAATTAAACTAGAGTTGTTGACCTTTGCACAGATAGAAGCAGTAGGATCCAACTGGTATTTA | |
| GCTGCAATGCCAAAACGAGTGCAGCTGGTACCCTGATGTCCAAGCGAGGNTTACTGAAGTGTCAAAATCTTCACATACTT | |
| TCTGATAAATTTGATCCTCCAAATTCTGTCCCATTATTTACATTTGTGTGNAGCTGGAAGTCCCCAGTCCTGTAGCCGAC | |
| NGCA | |
| Clone 0043-BGH Sequence | |
| TTTTTTTTTTTTTTTTTTTTTAAGTTAACAAGCTTGCATTTAATAAGTCTGAAACCATTCTCAGCACATGGCATTGTACA | |
| CGGGCATCTGTGCAAACAGATTCATTTAACAGGTCGTAGTTTAAAAAAGTCATAGATACTGTGAGTTCTGTATAAACCGG | |
| TGGACGGCAAGTTAGTTCCTTTTGATTTATAAGCCTCAATGTCACCGCAGAATAAAGAATGTAGCCAAAGAAAGCATTAT | |
| CGGTCACTCGTATAGGACAGWGTTGTTTCTATAATTTGAAGCTTTCTGAATGGACGGGTTCAGGCCTGATCCAACTGTAA | |
| AAAGATCACTCAGTGAATAGACTATATGGGAACTGTACAAAGTGTCATTAACTTCCATCATTAATAGCTTACTCAGCACT | |
| ATACCACTATTGCTAGTTAAAATAACCTGCTTCTGAGGCCCCACGGAGGGAGGCGGCCTGTGCACGCAGCCTCGATGCCC | |
| TGGCCACCCTCATCCCCAGGGCGTGCCATACAGTCCAACAGAAAACTTTGGCTTTAGGAAGGAATCACAGACATTGAAAA | |
| GAATGGCTTTAATCATTATTAAATGTGCAGTGGGAAGGAGTGTGCTTCAGATAGTCTGGGCAGGGCTGGCGGCAGGCAGG | |
| TCACTCCGCTGCACAGCTGCAGACACTAGTTTGTCATGACAAGACAATGAGGGAAAGCAGNC | |
| Clone 0044-Contig | |
| AAAGAAGAAGAGGGGGCTAAGCTGAGTATAGAGGTGCTCCAGACCAGCCTGCAGAAGGAACTCACTCTAAACAAAGGCCA | |
| GGCCTCCGCCATGGAGCTGCTGCGCTGCCCCACGCTTCGGCGCCTCTTCCTCTGCCTCTCTATGCTGTGGTTTGCCACTA | |
| GCTTTGCCTACTACGGGCTGCTCATGGACCTGCAGGGCTTTGGGGTCAGCATGTACCTTATCCAGGTGATTTTCGGCGCT | |
| GTGGACCTGCCTGCC1AGTTTGTGTGCTTCCTAGTCATCAATTCCATGGGCCGCCGGCCTGCACAGTTGGCCTCCCTGCT | |
| GCTGGCAGGCATCTGTATCCTAGTGAATGGCATAATACCGAGGGGCCATACAATCATTCGCACATCCCTGGCTGTACTAG | |
| GGAAAGGCTGTCTGGCTTCCTCTTTCAACTGCATCTTCCTGTACACCGGAGAGCTGTACCCCACAATGATTCGGCAGACG | |
| GGCCTGGGCATGGGCAGCACCATGGCCCGGGTGGGCAGCATAGTGAGCCCACTGATAAGCATGACTGCCGAGTTCTACCC | |
| CTCCATACCTCTCTTCATCTTCGGCGCTGTCCCCGTGGCCGCCAGCGCTGTCACTGCCCTGCTGCCAGAGACCTTGGGCC | |
| AGCCGCTGCCTGATACAGTGCAGGACCTGAAGAGCAGCAGCAGAGCAAAGCAGAAGCAACAGCAGCTGGAACAG [symbol] AG | |
| CAGATGATACCACTCCAGGTCTCAACACAAGAGAAGAACGGACTCTGAAAATGGAGAGGGCGTCACTCAGCAC [symbol] G | |
| AGTGGGGTTCTGCAGGTCCCACCATCTATGAGGAGGGGGAGTGAGTGGAGGGACTGGGCCATCTAAATGCGGAG [symbol] C | |
| ATTCAGAGAAATGCCTCCCCAAAGGTCACTTCAATAGACCTACTAGGAACAAAAGCTCTAACTGTGTGCAGCTTC [symbol] AG | |
| CAGAATGAGCTCTTCCTCAGCCATCTTCCAGCTCAGTGGTCACTCCTCCAGCTCCTGTCCCTTCCACCTCCAGGACATTG | |
| CAAAGAATCCCAGACAATTAAATAAATTTCTTCTACCTTGAAAAAAAAAAAAAAAAAA | |
| Clone 0045-Contig | |
| AGCAGGAACTGAAGTCTTTATGGCCTTCCCACTGGAGACCAGCCGCCTGGAAGGCAGATGGGAGCCTCTTTTTCCTGCAA | |
| TACAGTCGATACAGTTTGCTGTCGCTACTCTGAGTGTTATAGGCTCCAGTTCACTCCTCACCTACATCGCGTTCCAGAGC | |
| CCACAGAAGTCTGCAGAGATAAAGCCACTCTTGTATCTGAGCTTCTCTGACCTGCTCCTGGGAATTTGCTGGCTCGTAAA | |
| GGCACTTCTCTATGGAACTGCAGCAGCCCACAAGGACAGCATCTGCTATAACCTACAGACCGTGGGGGAGATTTTCTACC | |
| TTGCCTCACTCCTCTACACCGTCAGCTACATCTCGTGTCTCTACTCGGAGCTGAGGAGGAAGAGCCGCCAGACTGAGAGG | |
| AGCACGGTTGCCCAGGTGACAGATTATGTTTGCCAAGGTGGTCACATATTGTTCCTTTTGTCAAGCCTGATACCTCCGCT | |
| GCTGATGACGCCTGTGTTCCTTTTGGGCAATGTCAATGAATGTTTCCACAACTTCAGTGAGAGCCACAGGTGTATCCTAA | |
| TGCACTCACCGCCATCAGCCACGCCTGAGCCCCTGCCTTCCACCAATACATCTGTCTGCAGCACACTCTATTTTTATGGT | |
| GTCACCATTTTCCTGGCCAGCTTTTTCTTCAGCTTCCTCACCATCATGGTCTTACTCGTCCATGCCCAGATGTTATATAA | |
| GAAGTTTTGTRAAGTCGACGGGCCTTCTGGAGAGTGAGCAGTGGGCAGTGATTCACACTGTGGGCCAGCGAGTGCGTTTC | |
| TTNNCAGTGGCCTTTGTGTGCTGCTGGGGTNCAGCTGTCTCCCTGCTGATCATGAAGCTGACAGAGCCTCAGGAGACCTT | |
| CCTGCACATGGCCCTCTCTGTGCTCCAGGCTCTCTCAGCAGCATCCCAGGGGCTGCTCAACTGTGGCWTTTATGGCTGGA | |
| CNCAGTCCAAGTTCCAGCAGATAAAGCGTGAGCCTCACCGTGACGCAGACACGCAGACCCCWCTGCTGTCCTCTCAGAAG | |
| AGAATTTATAGTAGAAGCCCAAACCTACTGGAGTCTCCTCTTGCCCTTGCTTCCAGCTCCTCCACCGTTCTTTGAGACCC | |
| TAGAATGGGAATGTCAGGGAGTGAATGTTTCACATGAGTGGACTCTCCTCTGGCCAAACCCGAGACTGGAGAGCTGAAAG | |
| GAAATGTGGGCTCAGGGTCAGTAGCAATTCTGGGTGAATTCAGAAGCAGCTCTCTAACTGCTCCTTCAGAGATATACAGC | |
| CGCTTTCCATMTAAGAGACTCGTTACTTTGACAAGAAAAAATAAATCTCAGGTTCATAAAAAAAAAAAAAAAAAAA | |
| Clone 0046-Contig | |
| AAAAGTGCGCCCCGAGGTCGCGTCTTGCCCAAGTCTCCGCGGTCCCCAGCGCTCGCTCGCGCGGTCCTGCCACGGCCTTC | |
| CTGCTGCCCGCGCCATGGGCCTCAGCTCCAACTCCGCTGTGCRAGTTGAGTGGATCGCGGCCGTCACCTTTGCTGCTGGC | |
| ACAGCCGCTCTCGGTTACCTGGCTTACAAGAAGTTCTACGCTAAAGAGAATCGCWCCAAAGCTATGGTGAATCTTCAGAT | |
| CCAGAAAGWCAACCCGAAGGTGGTGCATGCCTTCGACATGGAGGATCTGGGGGATAAGGCCGTCTACTGCCGATGCTGGA | |
| GGTCTAAAAAGTTCCCCTTCTGCGATGGGGCTCACATAAAGCACAACGAAGAGACTGGCGACAACGTAGGACCTCTGATC | |
| ATCAAGAAAAAGGAAACCTAATGGACACTTGCGAGGCTGCACCCAGCGTGTTGTGATGTCACCTGCTGATTTACGTAGAA | |
| TGGCACCCAACCCACMBGTCTGATTGGCCTCCCCGGTTCTAGATGTGGTTGGTCCCTGCAAATCACAGCTCTCATATCCA | |
| TGGCATCGGCCTTGCTACTGAAACATGTGGTGCACGTTTGTTGAAAGAAGAAGAAAGGCTAAACCAACCTCGTGCTATAT | |
| GGGTTATTTTGGTCTTGTAAGGATCCGTTCCTTTAAAATAATGGTCTTAGAATATAGTTGTATCTTGAGGTTAAAGTATT | |
| AAATTATTCCAAAATCATGTACATGTAAAAAAAAAAAAAAAAAA | |
| Clone 0047-Contig | |
| AAAGGAGCAACGCGGGTCTTCCCGCTGTTGCTTGTCGCGGCCACGGCCGAGCATACGTCCCCGGCCTGAGGTGGTGGTGG | |
| CGAGCCCACCGCCGTTTGCTGCGACCTCATGGAAGGTGGCGGAGGAAGTAGCAACAAATCCACCAGCGGGTTAGCTGGCT | |
| TCTTCGGAGCCGGAGGAGCGGGTTACTCGAACGCTGATTTGGCCGGCGTCCCGCTCACTCGTATGAACCCCCTCTCTCCT | |
| TATTTAAATGTGGATCCACGCTATCTCGTTCAGGATACTGATGAATTTATTTTGCCAACTGGAGCTAATAAAACCCGAGG | |
| CAGATTTGAACTAGCTTTCTTTACCATTGGAGGATGTTGCATGACAGGGGCCGCATTCGGGGCAATGAACTGTCTTCGTT | |
| TAGGATTGAAGGAAACCCAGAGCATGGCCTGGTCCAAACCAAGAAATGTACAGATTTTGAATATGGTGACTAGGCAAGGA | |
| GCACTTTGGGCTAATACTCThGGCTCCCTGGCTTTGCTCTATAGTGCTTTTGGTGTTATCATTGAGAAAACACGGGGTGC | |
| AGAAGATGACCTCAACACAGTAGCAGCTGGAACCATGACCGGGATGYTGTATAAATGTACAGGTGGTCTTCGAGGAATAG | |
| CACGCGGTGGCCTGGCAGGACTGACACTCACCAGTCTCTATGCACTGTATAACAACTGGGAGCACATGAAAGGTTCCCTG | |
| CTCCAACAGTCACTCTGAAGATTTTTGGCACCTCAGGAATGGGGGACACTTCATAGTCATCCAGATCAATTAATAAGCCA | |
| GTCTGGAGTTGCTTTCTGTCTTGTACCTACAGTTACTTTGAACAATTCGAGACTCTGATTTGCTGTGACAAAGATCATGG | |
| ATGGTTAGCCTGGACTGCACTGCTTGCCCTCAGTGAGTGGAAGCCAACCCCTTGGTGGCTCACTGAGTATGTGTTCTCTT | |
| CTCCTTTGGAGATGATCTTGCACCCCAAACTGGAAAACCACTTACTCTCCAAATTCAGGCCATACTTTTTATTACCATGT | |
| TAATTCATTTAAAAAATCTAAAACTTAAATGTTGCCCTGTTTTCCAAATAAAGGGTGAAAACAGAAAAAAAAAAAAAAAA | |
| Clone 0048-Contig | |
| AGGGCCTCCGCACTTCCAAGTCATCTGCCGGCTACTTGGCTACCAGGGCATTGCAGTAGTCATGGAAGAGCTGCTGAAGG | |
| TTGTCAAGAGCCTGCTGCAAGGCACAATCCTACAGTACGTGAAAACTCTGATGGAAGTGATGCCCAAGATCTGCCGGCTT | |
| CCGCGACATGAGTACGGCTCWCCTGGCATCCTGGAGWTCTTCCACCACCAGCTGAGGACATTGWGGAAGWATGCASNAGC | |
| TGAAAACCGTATGCTTCCAGAACCTGCGGSAGGTGGGMAATGCTGYYCTCTTCTGCCTGCTTATWGAGCAAAGCCTGTCT | |
| TTAGAAGAACTCTGTGACCTGTTGCATGCAGCTCCTTTCCAGAATATCTTACCTCGAATCCATGTAAAAGAGGGGGAGAG | |
| AGTTGATGCCAAAATGAAAAGACTAGAATCCAAGTATGCCCCACTGCACCTTGTCCCACTGATTGAAAGGCTGGGGACCC | |
| CACAGCAAATTGCAATTGCAAGAGAGCGGGACTTGCTGACCAAGGAGCGTCTCTGTTGTGGTCTGTCCATGTTTGAAGTC | |
| ATCCTGACACGGATCCGGACCTTTCTGGATGATCCCATCTGGCGTGGGCCCCTACCCAGCAATGGTGTCATGCACGTGGA | |
| TGAGTGTGTCGAGTTTCACAGACTATGGAGTGCCATGCAGTTTGTTTACTCCATTCCTGTAGGGACACACGAGTTTACAG | |
| TGGGAGCAGTGTTTTGGAGATGGGCNTCCWCTGGGCTGGCTGCATGATCATTGTACTTCTTGGACAGCAGCGGCGCTTTG | |
| CTGTGTTGGATTTCTGCTATCATCNTCTCAAAGTTCAGAANCATGATGGCAAAGATGAGATCATCAAAAATGTGCCATTG | |
| AAGAAGATGGTGGAGAGGATCCGCAAGTTCCAGATTCTCAACGATGAAATCATCACTATCCTGGACAAGTACTTGAAATC | |
| TGGTGATGGCGAGAGCNCNCCTGTGGAGCATGTACGCTGCTTCCAGCCNCCCNTCCTCCAGTCCCTAGCCNGTAGCTGAG | |
| GGCTAAGCTCNCAGCATCCCAGTAACTGATGGCATGTTTGTCTTTATGTAAACTATATTGAAATTTTTAGGGGCTATTTT | |
| CCNTTATGTCTGAACCTACTTTTGATCTGAAAGCTTAACTTTATAAAATTTACTTATTTTTCTATACTAAAATTGTATAT | |
| GCCTTTGGAATTGGGAAGTCTGAGAATACGGTTACTAGTTGTTGCCNTTACGGTATTAACAAGTATTTTCATCCCGAATA | |
| TTACAGCAGNTA | |
| Clone 0051-T7 Sequence | |
| AAAGCCTGTGTACGCCACCATCGGCTYYNGGTATCGTCAACACGGCCTTCACTGTGGWGTCGCTGTTTGTTGTAGAGCGA | |
| GCTGGACGACGGACCCTGCACCTCATTGGCCTGGCTGGCATGGCAGGCTGWGCTGTGCTCATCACCATCGCCCTGGCCTT | |
| GCTGGAACCGCTGCCTTGGATGTCCTATCTGAGCATCGTCCCCATCTTTGGCTWWGTGGCCTTCTTTGAAGTAGGCCCTG | |
| GTCCTATTCCATGGYTCATTGTGGCCGAGCTGTTCAGCCAGGGGCCCCGTCCTGCTGCTATTGCTGAGGCTGGCTTCTCC | |
| AACTCGACCTCAAACTTCATTGTGGGCATGTGCTTCCAGTATGTGGAGCAACTGTGCGCCCCCTACCTCCTTCATCATCT | |
| TCACGGWGCTCCTCGTGCTCTTCTTCATCTTCACCTACTTCAAAGTCCCTGAGACCAAAGGCCTGAACCTTCGATGAGAT | |
| CGCTTCCGGCTTCCGGCAGGGGGGTGCCAGCCAAAGTGACAAGACACCCGAGGAGCTCTTCCACCCTCTGGGGGCGGACT | |
| CCCAAGTGTGAGGAGCCCCACACCCAGCCCCCGCCTGCTCCCTGCAGCCCAAGGATCTCTCTGGAGCACAGGCAGCTAGA | |
| TGAGACCTCTTNCGAACCGACAGATCTCGGGCAAGCCGGGCCTGGGCGCCTTTCCTCAG | |
| Clone 0052-T7 Sequence | |
| AAAGCTTACCCAGCAGSRNGAGTAGATTTGTATCAACAAGAGAAGGCACGGATGGGCTCAGCGGGACTCAGAGTTCTTGC | |
| CTTGGCGTCTGGTCCGGAACTGGGGCAGCTGACCTTCCTTGGCCTGGWCGGAATCATTGACCCTCCTCGAACTGGTGTGA | |
| AGGAAGCTGTCACAACACTCATTGCCTCAGGAGTCTCCATYAAAATGATCACTGGAGATTCTCAGGAGACTGCAATTGCC | |
| ATCGCTAGWCGTCTGGGATTGTACTCTAAGACTTCACAGTCCGWGTCTGGGGAAGAAGTCGATACAATGGAGGTGCAGCA | |
| CCTTTCACAGATAGWGCCAAAGGTTGYAGTATTTTACAGAGCAAGCCCAAGACACAAGATGAAAATTATTAAGTCTCTAC | |
| AGAAGAACGGGGCAGTTGTAGCCATGACAGGAGATGGGGTAAATGATGCAGTTGCTCTCAAGGCTGCAGACATTGGAGTT | |
| GCGATGGGCCAGACTGGCACCGATGTTTGCAAAGAGGCTGCAGACATGATCTTGGTGAATGATGATTTCCAAACCATCAT | |
| GTCTGCAATAGAAGAGGGTAAAGGCATTTATAATAACATTAAAAATTTTGCTAGATTTCAACTGAGCACGAGTATAGCAG | |
| CATTAACTTTAATCTCATTGGCTACGTTAATGAACTTTCCTAACCCTCTCAATGCAATGCAGATTTTGTGGATCAATATT | |
| ATAATGGATGGACCTCCAGCTCAGAGCCTTGGAGTAGAGCCAGNGGATAAAGATGTCATTCGAAAACCGNCTCGAAA | |
| Clone 0052-BGH Sequence | |
| TTTTTTTTTTTTTTTTTTTTTTTTTTTTTTTTTTTTTTTTGCCYTTAAATGATTGAGTTTATTTGTGKGTTAGTAAGAAA | |
| GCCCYAYAACCATAAATAGTACAATATTTAAAAGTAAAAAAAATATTTATATCCATCTAAGACAGACAGTGTATTTGTAC | |
| ATTAGAATTCTTTAAGTGCAGAAGGTGGTTCAGGTTTTGCCTTTTGKATTTTTTAATTCAGTACTTGCCCATATGTTGAA | |
| GTTCACTCAATTGCAAAAGCCAATTCAAATCAAGGAATGTGTTATTCAAAGKTGCATAATTTCATTTGGGACTACCAACA | |
| AAGTTCAAATCAAGTCTTTAACATTAATGGGTCATTTTTAGTCAATGGAAAGTTAAAAAGTCCTTAAGTCTTCATTATCT | |
| TCTTGAACTTGCCCTTCTAAATGATTCTNAGACTGCANTNCCTAGTTTGCNAATTAAAAAAAAATGCNNTATGCATCATA | |
| CTTNAAGGAAAGACGATGAANGCTNAACCAGCNTTCTTCCTGGACCTTCTCCCGGCTCCTTTCANCCTTCTTTATAACCC | |
| TCAGNCACA | |
| Clone 0055-Contig | |
| AGGATTGGCGGGGTTCTCTTTGACCACATAAATCAATTCTTTCTTTTGGGAGCCTCAATGGTGGCAACTGCAGCGGGACT | |
| TTATCTCATCCCGTTCTGCAAGACAGCAGTCTTATTGATCATCACCATGTCTGTCTTTGGAGCTTCCGTTGGTGTTGTGG | |
| ATACAGGTGCAAATGTTCTCATCTTGGATCTCTGGGGGGACAAAGGAGCCCCACAAATGCAGGCCTTGCACTTCAGTTTC | |
| GCCTTGGGTGCCTTTCTGGCTCCCCTGCTGGCTAAGTTGGCCTGGGGTACAGCACCTGCTCAGAACCACACCGAGTCCGA | |
| CCTTGACACTCTGATGCTGAACCGATCCTCCAACGGCACCTCAGACTCCGTGTTTGCGGTACCCGATGACATGAATCTGC | |
| TGTGGGCATATGCTTCCATTGGCACCTTTATTTTAGTAGTTTCTGTCTTTCTGTTTGGTCTGTTTTGTAAGAAACATTCA | |
| AGCCAGAAAAAACCCAGAGCATCTGCTGAGGGAGCTCGAAGGGCTAAATATCACAGGGCCCTGCTATGCCTGCTCTTCCT | |
| CTTCTTCTTCTTCTATGTGGGAGCCGAGATAACATACGGCTCTTACATATTCTCCTTCGCCACCACCCATGTTGGCATGG | |
| AAGAGAGCGAGGCAGCTGGCTTGAATTCCATCTTCTGGGGAACCTTTGCAGCCTGCAGGGGCCTGGCCATCTTCTTTGCG | |
| ACATTCTTACAGCCTGGAACCATGATCGTGCTGAGCAACATTGGCAGCCTGGTCTCATGTTTCTTTCTGCTACTTTTTGA | |
| CAAGAGCCCTCTTTGTCTCTGGATCGCGACTTCTGTGTATGGAGCCTCAATGGCAGCCACGTTTCCCAGCGGCATCTCCT | |
| GGATTGAGCAGTACACCACCTTAACTGGGAAATCTCCAGCATTCTTTGTAATTGGCTCTGCCCTGGGAGATATGGCCATT | |
| CCAGCGGTGATCGGAATTCTTCAGGGACACTACCCAGATCTGCCAGTAGTTCTGTACACATGTCTGGGCTCAGCCATATT | |
| CACAGCTATTTTATTTCCTGTGATGTATAAATTAGCTACCTTGCCCCTGAAGAGAGAGGACCAGAAAGCTTTGCCCACTA | |
| GTTCTAGACTGTGAGGAAGAGACTACATGAGAACTTAAAAAAAAAAAAAAA | |
| Clone 0056-Sequence | |
| ATGCAATGGCGGACGTGTCTGAGAGGACGCTGCAGGTGTCCGTGCTAGTGGCTTTCGCCTCTGGAGTGGTCCTGGGCTGG | |
| CAAGCGRATCGGCYGYGGAGGCGTTACCTAGACTGGAGGAAGCGGAGGCTGCAGGWCRRGCTGGCAACGACTCAGAAAAA | |
| GCTGGACCTGGCCTGAGCACGCGCTGCAGCCCGAGTCCGCCGGGTTCTCACTCCCTAAGCCCAACGCAGCCCGGATCGTG | |
| GGAGCCGCGCGACCCAGGAGTCGTCCTTGCACGGCTTGCAAGAACATGGCTTGCTTCAGAAAGAAAATAGTTTTGTCTTC | |
| TCTAACAACTTACTTTCAGCTTGTCGAAGATGAAAATAAAAAGCACTGGAGAGAAATAATTTCTTGCACTTTATGAATCT | |
| ATTTTTAAAATAAAAAAATTAAACATCTTTGAATCTTTTTCCTCCTCACAAAAGAAAGCAGTATTTTTGCCTACCATTCA | |
| GTTTGCTGCAGTAAGAGATTTGGAGCCTGAAAGCAGAGACTTTCTGATGGAATCTCACCTTGGTACAGCCTGGAGGCAGA | |
| TCTGATCAACGGACCATTATGAGTCATTTTTCTAGACATATTCAGAAAACCTAGGAGCTGTGTCAAATGCCTGAATTAAG | |
| CATTACAAATGCAAGATATTTGCACTTTGAAGAATGTAGAGAGTAAAAAAACTAAAATTAAAAAAAATAATGCATGTGAT | |
| ATAACGGAATATATATGTGAAAGAGAAAAAAAAAAAAAAAAAAAAAAA | |
| Clone 0057-Contig | |
| AGATAGTTTTGAACTTCCGATTCCCCTGCCTCCCCACCCCAGGGCTGTGATTGCAGGTGTGCGCTTGGGACTGACCCCAG | |
| GCTTTGTCGGTGCTAGGGCAGCACTCTGCGACTGAATTAGGTCCCAGCCACTTCTCTGTCTTTTAAAAGAACAAAACATT | |
| GCTAAATGTGCCATTGTTGCTTTGAGTTTTAATTCTTTTTTTTTTCTTTCTTTCATAAAACATTACAGTCTTAAGATATT | |
| AAAGACTTTTATTCTGGTTCTATTTCTGTCTTTTCACTCAAAACTGGTTTTACAAATGATGCCTTGTTTACAGAAAGCTC | |
| TCTACCACAGGGCCTAGTCATGTGTAAAGTCTCAGTTTCTCTCTGGAGTATCTTGGAGCCTAGCACACTGGCTTTAAAGG | |
| ACACAGCTAAGAAGCTGATATCTTGACAGTGTTTGTAGACCTTTGTTATAAAAATGAATGTCCTGGAAAGGGTTGGGAGG | |
| GAGTTCAACAACAAAGAAACAAGAATGTCATGTTTAAATTTAATAGTTGTCTAAAATGTCATCTCAAGTCAAGTCACTGG | |
| TCTGTTTGCATTTGATAGGTTTTTATACTAACTAGCATTATAAGATTATTTCATAATTAGAAAATACCTGTGGATATTTG | |
| TATAAAAGTGTGAAATAAATTTTTTACAAAAGTGCTCATCGCTTGTTAACACAGCATCATGTATGTGAAAGCAAACTCTA | |
| AGATTATAAATGACAACCTGAGTTGCCTTTCTTTGTATTTCATCAAGCCAAAGTAAAGCTTTCAATATTTAAAAAAAAAA | |
| AAAAAAAAAAAAAAAAAAAAAAAAAAAAAAAAAAAA | |
| Clone 0060-17 Sequence | |
| AGCATCCCTCCATGGCCTCTGCATTGGCTYCTGCTTTCTGACCTGCTTGAGTTCCAGTCCTGACTTCCTTGGTGATGAAC | |
| AGCAGTATGGAAGTGTAAGCCGAATAAACCCTTTCCTCTCCAACTTGTTTCTTGGTCATGATGTTTGTGCAGGAATAGAA | |
| ACCCTGACTAAGACAAATTGGTACCAGCAGAGTGGGGTATTCCTGTGACAACCTGACCATGTTTTGGGGAGGACTGTGGA | |
| TGGACTTTGGAACTTTGGGCTTAAAGATCCATCCGTTGTTAAGAGCTCTGTCAGATGTTGTGTAGGAGCTTGGAAGATAA | |
| TGTTGAGAACACTGCAGAAGATGGAGGTCTGGTTTGTGAAATTTCAGAGGGAAAATTAAAGACTCTTTTCAGGGCCATTG | |
| CTGTTTTGAATGTGAAGATTCTGTAGTTCTGGWTAGCTGGGGCTGAAGAATCAGCTGTGATTAACAAGATACCAGAACTA | |
| CCAAAGCAAAAACTTTGCATTACTGGGACTATTGATGCTGGTTAGCTGGAGCTAAGAAATTAGCGGTGATTAAGAAGAGA | |
| CCAGCATCATTGAGGTGACATCTTCTGGGAAGTGTTTTCTGAAAGCACAAAGATGCTGTGTTCCAGAGATGGCCAAGGTT | |
| GTACTCCTGCTGCAGCAGGACTTGGTAATATGTAAGGGTCACCCAGGTGGTACTGGTTTTGAAGGCATGAANGGGTCACG | |
| CAAAGCAGCTGANGCTCGGCACTGTGAGAGGCCATGGAAGGCCATTGCTGAAAGTACAGCCTCAGTT | |
| Clone 0063-Contig | |
| AAACGGCAACACTTCGGTCAGGATATCTTCTACCAGACACCAAGGCGTATGGAGATAGAATAGAAAGAATGCTTCGCCTC | |
| AGTTTAAACATTGACCCTGAAGCACAGGTGGAGGAAGAACCAGAAGAAGAGCCTGAAGACACCTCAGAAGACGCAGAAGA | |
| CTCAGAGCAGGATGAGGGAGAAGAGATGGATGCAGGGACAGAAGAAGAAGAGGAGGAAACAGAAAAGGAATCTACAGAGA | |
| AGGATGAATTGTAAATTATACTCTCGCTATGAATCCCGTGTGGAGAGGGAATGTGAAGTTTTGAAGTCATTTCTTTTGAG | |
| AGACTTGTTTTGGATGCTTCCCCAAGCCYCCTTCTCCCCTGCMCTGTAAAATGTTGGGATTATGGGTCACAGGAARAAGT | |
| GGTTTTTTTAGTTGAATTTTTTTTAACATTCCTCCTGAATGTAAATTTGTACTATTTAACTGACTATTGGTGTAAAATCT | |
| TGTCATGTGTATAAAAATAAAAAAAGRTCCCCAATWAAAAAAAAAAAAAAAAAAAAAAAAAAAAAAAAAAAAAAAAAAAA | |
| AAAAAAAAAAAAAAAA | |
| Clone 0065-Contig | |
| AGGGGAGATCCTGAGAACAGACCAGTGGCCTCGGAGGCTCTTCATGCAGCTCATTCCGCAGCAGTTGCTGACCACCCTCG | |
| TGCCACTGTTCCGGAATTCACGCCTGGTACAGTTCCACTTCACTAAGGACATGGAGACTCTGAAGAGCCTTTGCCGGATC | |
| ATGGACAATGGCTTCGCGGGCTGCGTGCACTTCTCCTACAAGGCATCGTGTGAGGTGCGTGTGCTCATGCTCCTGTACTC | |
| CTCGGAGAAGAAGATCTTCATCGGCCTCATCCCGCACGACCAGAGCAACTTTGTCAACGGCATCCGTCGCGTCATCGCCA | |
| ACCAGCAGCAGGTCCTGCAGCGAAGCCTGGAGCAGGAGCAGCAGCAGCGAGGGATGGGTGGCTAGAGGATGCCTGGGCTG | |
| GGCGGGCCACAGTCCCAGACGAGGCCCCAGTGGAGACTGGTCAGCTGCTTCTGAGCAGGGGCTCCTGGCGACTGTACCTG | |
| CCCAGCAACCTGGAGGACAGCGTCCTGAGGTCTCCCAAGGATGGTCCTTGCCCCTGTATGTTTCCCTAATAAAGCCTTTT | |
| ACCCCAAAAAAAAAAAAAAAAAAAAAAAA | |
| Clone 0066-Contig | |
| GGCTGCTTCGTGGAGCAGAGAGGTGCATCACCAGGTTCCCGATGAACCCAGAGAACCCTCCACCGTATCCGGGCCCCGGG | |
| CCAACAGCCCCATACCCACCTTATCCACAACAGCCAATGGGGCCAATGGGGCCTATGGGAGCCCCACCTTCCTCAGGGGT | |
| ACCCCTACCCACCACCTCAGGGGTACCCCTATCAAGGATACCCACAGTACGGCTGGCAGGGTGGACCTCAGGAGCCTCCT | |
| AAGACCACAGTGTATGTGGTGGAAGACCAAACGAAGAGACGACCTGSGCCCATCCACCTGCCTCACAGCCTGCTGGACTG | |
| CTCTGTGTTGCTGCTGCCTCTGGGACATGCTCACCTGATCACCTGATGAGCCCAGCTCTTCCGCTTGGCCGCTCTGTGCC | |
| ACCTCCGATAAGTGTGCCTGGCCCCATCTCTTCTGATTGCTATAAAGTGGCTAGCTCTGCGCAGACACCTCTACTTTCTG | |
| TCCTATGGAGTTTTAGATTAGTGAGGGTTACTGCTATTTAGTTCAGTGACTTGATCTTTTTAATGTTCAAAACTCATTTC | |
| TTACTGATCTTTAAAAAATGTGCTAAATAATTTACTTTTTTGGCCAAAGGCTTAGTTATGAAATATATATTTATAATGTT | |
| TAAATTATACACTCAAGGTAATGGCAATATGTGACACACTACTAGATCTCTCTGCTCATACCCTTTATGTTTCAATAAAT | |
| CTGTCCAAAATCTGGAAAAAAAAAAAAAAAAAAAA | |
| Clone 0067-Contig | |
| CTGGAAAGGCGCGGAGCCTGCTGCTGCCATGGWGGCTGGTGGCTGGGTACACTATGTCTAGACTGGGGGCCCTGGGCGGC | |
| TCCCGCGCCGGGTTGGGACTGTTACTTGGTACTGCCGCCGGCCTTGGATTCCTGTCCGTCCTTTACAGCCAGCGATGGAA | |
| ACGGACCCAGCGCCATGGCCGGAGTCACAGTCTGCCCAACTCCCTGGACTATGCGCAGGCTTCAGAGCGTGGACGCCAGG | |
| TGACACAGTTTCGGGCTATCCCAGGTGAAGCTGGAGATGCTGCCATACTGCCCAGCCTCTCACAGGAAGGGYAGGAGAAG | |
| GTGCTGGACCGCCTGGACTTTGYGCTGACCAGTCTTATGGCGCTGCGGCGCGAGGTGGAGGAGCTTCAGAGAAGCCTGCA | |
| AGGACTAGCTGGGGAGATTGTCGGKGAGGWCCGCTCTCATATAKAAGAGAACCAGAGAGWGGCCCGGCGGCGCASGTTCC | |
| CTTTTGCCAGAGAGAGGAGTGACTCCACGGGCTCCAGCTCTGTCTACTTCACCGCCTCCTCAGGGGCCGCACTCACAGAC | |
| GCCGAGAGCGAGGGAGGCTATACAACAGCCAACGCGGAGTCTGATTACGAGCGGGACTCCGACAAGGAGAGTGGAGATGC | |
| TGAGGACGAAGTGAGCTGCGAGACCGTGAGGATGGGGAGGAAGGACTCTCTGGACCTGGATGTGGAGGCGGCGTCCAGTC | |
| CGTC | |
| Clone 0068-Contig | |
| AGCGCTGGGTCTGAGTGACCAAAGGCAGTAGCGCTCGCGGAGATCACCCGCTGGCCCTCGATCACCATGTCGGCCTTCGA | |
| CACTAACCCCTTCGCGGACCCAGTGGACGTAAACCCCTTCCAGGATCCCTCTGTGACCCAGCTGACCAATGCTCCTCAGA | |
| GTGGCCTGGCTGAGTTCAATCCCTTCTCAGAGACAAATGCAGCGACAACAGTTCCTGCCACACAAGCTCCTGGGCCCTCC | |
| CAGCCAGCAGTTCTCCAGCCCTCAGTGGAACCAGCACAGCCAACGCCCCAGGCTGTTGCAGCTGCGGCCCAGGCAGGCTT | |
| GCTTCGACAGCAGGAAGAACTAGACAGGAAAGCTGCCGAGCTGGAACGCAAGGAGCGAGAGTTGCAGAACACTGCAGCGA | |
| ATTTGCATGTGCGAGACAACAACTGGCCGCCACTCCCCTCATGGTGCCCTGTGAAACCCTGCTTCTATCAGGACTTCTCC | |
| ACGGAGATCCCTGCTGACTACCAGCGGATTTGCAAGATGCTTTACTATCTCTGGATGTTGCATTCAGYGACTCTGTTTCT | |
| AAACCTGCTTGCGTGCCTGGCCTGGTTCACAAGTGACGCAGCCAATGGAACAGCCTTTGGCCTCTCCATCCTTTGGTTTC | |
| TGATCTTCACTCCCTGTGCCTTCCTTTGTTGGTACCGACCCATCTACAAGGCTTTCAGGTCTGACAACTCTTTCAGCTTC | |
| TTCGTGTTCTTTTTTGTATTTTTTTGTCAAATAGGGATCTACTTCATCCAGTTGATTGGCTTGCCTAACTTGGGGACCAG | |
| TGGTTGGCTTGCAGCCCTATCTACGATGAAAAATGGGCCCTTAGCTGTGACCATCATCATGATGGTAGTGGCTGGCTTCT | |
| TTACCCTCTGTGCTGGGCTTTCACTCTTCCTCCTGCAGCGGGTACACGCCTTCTACCGCCGAACAGGAGCCAGCTTCCAG | |
| CAGGCCCAGGAAGAGTTCTCCCAGGGTATCTTCAGCAGCAGAACCTTTCGCGGGGCTGCCTCATCTGCTGCCCGAGGAGC | |
| CTTTCAGGGGAATTAGGCTTCCCGACTCTCCTTCCCTGAGCCCTGGCCTCTCTGTCAACTGTCTTCTGAGCTGCACTTTC | |
| CTCGGGTGCCTTATGCAGTGACTACACCAGCACAGACCTGGCAGGGATTTGTCTTGTCCCTGCTCCTCCCTCAGTAGTCA | |
| GCTTTCTCCTCTACAAGAGGGAAGGAGAGGAAGGGACAAGGACTTTAAAAAAAAAAAAAAAAA | |
| Clone 0069-Contig | |
| TTCGTCACTGCCAGGCTGAACTGGAGAGGCTTTCAGAGTCCCTGAGGAAGAGCATCTCCTGTGGAGCTTTCTCTGTTCCT | |
| GGGGGTCACAGCCTCTACTTAGAAGCCAGGAAGAAGATTGAGCTGGGCTACCAGCAAGTGCCGAGGAAGGGAGTGAAGGC | |
| AAAAGAGGTTCTCAAGAGTTTCCTACAGTCACAGGCTATTATGGAGGACTCTATCTTGCAGTCAGACAAAGCTCTTACAG | |
| ATGGAGAGAGAGCCATAGCAGCTGAGCGGACAAAGAAGGAAGTGGCTGAGAAGGAACTAGAGCTGCTGAGGCAGAGACAG | |
| AAGGAGCAAGAGCAAGTGATGGAGGCTCAGGAGAGAAGCTTCCGAGAAAACATTGCTAAACTTCAGGAGAAGATGGAGAG | |
| CGAAAAGGAGATGCTGCTGAGGGAGCAGGAGAAGATGCTGGAGCACAAGCTGAAGGTCCAAGAAGAACTGCTTATTGAAG | |
| GATTCAGAGAGAAATCTGATATGTTAAAGAATGAAATAAGTCACCTGAGAGAAGAGATGGAAAGAACAAGAAGGAAACCC | |
| TCACTGTTTGGTCAAATCCTTGACACCCGTTGGCAATGCGTTCATTATGATTTTACCAGGAGCTGGTAAACTATTTGGTG | |
| TGGGGCTGAAATTCCTCGGCTCACTAAGTAGTTAGTCTAAAGAGTTTCTGGGAAGGAATTTATAAAATCCAGGCTTCTGG | |
| TATTTTGATACCAAACTTTTTGCTGKGATTTTTAAGTACACATTCTAGTTTAATAACCACGGTTACMAAGAAAGTAAGAA | |
| CTTYCATTAATAGCTAAATTGTCAGATACTGCTTCCTCGGAGTCTAATTCTGTGTCAGTTCATTACCTATTATATTTAAG | |
| GTTAAAGATNCAAACCAATGATTGAAGAATATAAGGGTCTGGCGTCCATAAATCCTGTCAAATTTCAGTGATGTTATCTA | |
| AATAATAAAGAGGGAGGCCAGTCAGAATATAGTGAAATTATTTCTAGAAAAGATGTAAGTAAATCAAACAGAAACATGGC | |
| CAAATGAAGACMCATTGGCATATGTATCCCAGTTGTACACTACAGATAAATTGTTAAAAAGAGCTCTTGTTAAAACGCMC | |
| AAGTGGGTTTTCATAAGAGAATGTATGAGGAAAATGTTTGATTGATAATAAAAGGCATGCAGATTAAAATAAAAAAAAAA | |
| AAAAAAAAAAAAAAAAAAAAAAAAAAAAA | |
| 0 | |
| Clone 0070-Contig | |
| GAACGCCTCTGGGGAGCATGGCATCCTGGTTTTCTCTTTGGGATCCATGGTCTCAGAGATTCCGGAGAAGAAAGCCATG | |
| AAATTGCTGAGGCTTTGGGCAGAATTCCTCAGACGGTCCTGTGGCGCTACACCGGAACTAGACCATCGAATCTTGCAAA | |
| AACACAATTCTTGTCAAATGGCTACCCCAAAATGATCTGCTTGGTCATCCAAAGACTCGGGCATTCATCACACACTCTG | |
| CTCCCATGGTATTTATGAAGGAATATGCAATGGAGTTCCGATGGTGATGATGCCCCTATTTGGCGATCAGATGGACAAT | |
| CCAAGCGCATGGAAACTCGGGGAGCTGGGGTGACCCTGAATGTCCTTGAAATGACTGCTGATGATTTGGAAAATGCCCT | |
| AAAACTGTCATCAACAACAAGAGCTACAAGGAGAACATCATGCGCCTCTCCAGCCTTCACAAGGACCGTCCTATAGAGC | |
| TCTGGACCTGGCTGTGTTCTGGGTGGAATACGTGATGAGGCACAAGGGGGCACCACACCTGCGCCCGGCCGCCCATGAC | |
| TCACCTGGTATCAGTACCACTCCTTGGATGTGATTGGCTTCCTCCTGGCCATTGTGTTGACAGTGGTCTTCATTCTCTT | |
| TAAATGTTGTGCCTATGGCTGCCGGAAATGCTTTGGGGGAAAGGGGCGAGTGAAGAAATCACACAAATCCAAGACCCATT | |
| GAGAAGTGGGGGGAAGTGAAGGAGAAGTATTAGTTCATTATCTGATCAGTTGAACYTTGGAAACAAGTGTTAGATCCATA | |
| TTGTTTTTGTTAGGGAAATAATTCACCATACATTATACATTCAGCACATTTAAAAATAATAATAATAACAATCTAATTGC | |
| TGGCCACACCCATCAGGGAAGGTTCTAGTATATGTGATGTGCTTTTCCAGTATCTTCAGTCTAGACAACTCTTGCCATCT | |
| GTTGGTAATTTACAGAAAGTCTGGCACTCTGCTTTCAGTGACAGCCCCACAGTTTCCCCTGCTCCCCCCAGCTGATGGTT | |
| TTCTCCCCTGGATTCTCAGACTGCCGTGGCCTTCTCCAGTGTTAGTCATTCTTCATTGTGTTCATGCATTATGGGTAGCA | |
| AGACCTTTGGAGCTTTGGGAGAAGAGATCAGGCTGTGACACTGATGGCCCTGTGTTTGAGATAATAATTGTTGCTTGTGC | |
| CGGAATTTGATGAAAACCAAAGTATGTTCTAAGGCAAGTACATCTTCTATTGTGTTCCCAAACCAAGAACTTATCAATAA | |
| ATTCATATAAATTGTATCACTAAAAAAAAAAAAAAAAA | |
| Clone 0071-Sequence | |
| AGAGAAGCAGACATGGCTCAATTTGCACAGGTCATGGCTGAAGTGGGCGACTTTGGTCGCTTTCAGGTGCGGTTGACCAT | |
| CCTGATGGGCATTCCCAATTTCCTGGCTGCATTCTTCATATTTGGCCAAGTCTTCATGGTCCTTGATGAGGCTCACCACT | |
| GTTCAGTGTCCTGGGTTAAGAACCACACTTTCAACCTAAGTGCCGCTGACCACCTGGCTATAAGCATCCCCAACGACACA | |
| GCGGGCAGACCCGAGTCCTGCCTCATGTTCCGGCCACCTCCTGACAGTGCCAGCCTGGAAGACATCCTGAGCCACCGCTT | |
| CAATGAGACACAGGCCTGTGACTCAGGCTGGGACTATCCTGAGAACCGGCCTCAGTCCCTAAAGAAAGAGTTTGACCTGG | |
| TGTGTGATCGAAAGAACCTGAAGAAGACCTCCCAGTCGGTGTTCATGGCTGGTCTCCTTGTTGGGGCCCTGGTCTTTGGG | |
| CCTGTCTGTGACTGGATTGGCCGCAGACCCTCCCTCCTGATGCAGGTGCTTCTGTCAGGCATCACAAGCATGGCCACAGC | |
| CTTCGTGTCCAGCTTTGAGCTCTACTTGGCCCTACGCTTTGTCTTGGCTACTGCCAATGCTGGATTTTTACTAAGTACCA | |
| ATGTCCTGATTTCAGAGTGGGTGGGGCCATCTTGGAGAACACAAGCCGTGGTCTTTGGCCCANAGCAACGTTGCCCTTGG | |
| GCAGATGGTGTTAGCAGGACTGGCCTATGGTGTCCGAAACTTGGAGACTTCTTCAGATACANGGACCCGACCCGCTTACT | |
| GNTCTTCTTCTATTTCT | |
| Clone 0073-T7 Sequence | |
| AGCGGGCCTGAGCCGAGCAGCTGCGCGACGTCATGGACAACTCCGGGAAGCAGGCTGAGGCTATGGCGCTGCTGGCTGAA | |
| GCGGAGCGCAAGGTGAAGAACTCGCACTCCTTCTTCTCCGGCCTCTTTGGAGGCTCATCCAAAATAGAGGAAGCATGCGA | |
| GATCTATGCCAGAGCGGCGAACATGTTCAAGATGGCCAAGAACTGGAGCGCTGCTGGGAACGCTTTCTGCCAGGCTGCCC | |
| AACTACACCTACAGCTCCAAAGCAAGCACGATGCAGCCACCTGCTTTGTGGACGCTGGCAATGCTTTCAAGAAAGCTGAC | |
| CCCCAAGAGGCCATTAACTGTCTGATGAGAGCAATTGAGATCTATACAGACATGGGCAGATTCACAATCGCAGCCAAGCA | |
| CCACATCTCCATCGCTGAGATCTATGAGACAGAACTGGTGGATGTAGAGAAGGCCATCGCCCACTATGAGCAATCCTGCA | |
| GACTACTACAAAAGGGAGAAGAGTCCAACAGCTNAGCCAACAAGYGTCTGCTGAAGGTGGCTGGCTACCCCCGAACAGCT | |
| GGAGCAGTACCAGAAGGCTATCGACATCTATTGAGCAGGGGGGGACCAGTGCC | |
| Clone 0074-T7 Sequence | |
| AAAGTTGTTCTTCTCGTGGTTCCCAGTGGCGAGAGGAGGAGGAAGCCCGGAGCGGAGCGGGGCGKCTGGGGGGGGTGGAC | |
| CCGCCGCGGCTGCTGCTACCACCGCCGCCGCCGCCACCACCGCTCGTGGGGCTCGTGGCGTGAGGAAGGAGGACGAGTGA | |
| GACCCCGGGGCGAGCGGGCGGCGGCGCCGCTGCTGCTGCTGCTGCTGCGGGAGGGTCGGCGGCGGGACGGCGATGGCGGA | |
| TATCGACAAACTCAACATCGACAGCATCATCCAACGGCTGCTGGAAGTGAGAGGGTCCAAGCCAGCCAAGAATGTCCAGC | |
| TCCAGGAGAACGAGATCCGAGGACTCTGCCTGAAGTCTCGGGAGATCTTCCTCAGTCAGCCTATCCTTTTAGAACTTGAA | |
| GCACCACTCAAGATATGTGGNGACATCCACGGGCAGTACTATGATTTGCTCCGTCTGTTTGAATACGGTGGCTTTCCTCC | |
| AGAGAGCAACTATTTGTTTCTCGGGGACTATGTGGACAGGGGCAAGCAGTCCCTGGACACAATCTGCCTCTTGCTGGCCT | |
| ACAAAATCAAGTATCCGGAGAACTTCTTTCTTCTCAGAGGGAACCACGACTGCGCCAGCATCAATAGGATCTACSGATTT | |
| TATGATGAGTGTAAAAGAAGATACAMCATTAAGCTTGTGGAAAACGTTCACAGACTGKTTTAACTGCTTGCCGATAGCAA | |
| GCCATCGTGGACNAAGAAGATAWTCTGCTGTCATGGAGGGTTATCACCAGAWCTTCAATCTATGGAGCAGAATTCSGCSG | |
| AAATATKAGACCAAYTGATGTACCAGAWCAAGGGCCTTCTTTKCGAACCNTTGGGGGCCTGANCCCCATAAAGAGGCNTT | |
| AAGCTTGGGTGGAAAATGCCCGAGGAGNGGCCTTCCAATTGGTGCCAAAT | |
| Clone 0076-Contig | |
| AGGATCATCCGTCTCGCCTCCAGTTCTTCACCGGCTGAGCTGGGTACGGACATCTCAGTGACCACGAGCTAACATAGCAC | |
| GTGAAACTGACCTCAGGAAGAACGAAGCCTGGAGACCCGGAGTGCGACAGCGACCTCCGGACTGAAGTGGAAGAAGCGTA | |
| GCGAGGCCTTCGTGTCCTCGCTGTGGGGGGCGGGGCTTGTTGTGCAGGCGGGGCTCACGTGGTACTTTGGTACTTTGGAT | |
| GACTGAAGGGAAGCTTGGTACCCGCCTGGTCACTACCCGGAAGTCCAGCCGGAGTAGCACTACCCTGCAGTCCACCCGGC | |
| TGTGAAGCGGTGGTGCTCTTAGGGGTGCTTGACGCCCTCATTGTTAGAAACTGATGGGTACTGTTGGCCGATTCCTAGAC | |
| CCGCTTTTGACCTACGTCATAAATGTGTTTCCGGAACTCTGCCAAAAAGTGTTCTGGAGGTTTTTGTTTGTTTCGGAGCC | |
| AAGGCCTCCCTCGTAGCTCTTAATGGCCTGAAACACTCTTTGTAGCCTAGACTGGCATTGAAGTCAAGTTAGCCCTGCCT | |
| TTGCCTCCCTGGGCTCTGGGATTACCCTGCCTTTCTTGATTATTTCTATTTGAACAAATATGAACAAACTATGTGGCCAC | |
| CACAAAAAAAAAAAAAAAAAAAAAAAAA | |
| Clone 0077-Contig | |
| AGATGGAGCCTGCGTTTCACAGAGGGGATCTCCTTTTCCTCACGAACCGAGTTGAAGATCCTATACGCGTGGGGGAGATC | |
| GTTGTTTTCAGGATAGAAGGAAGAGAGATTCCTATAGTGCACCGAGTCCTGAAGATCCATGAAAAGCAAGATGGGCATAT | |
| CAAGTTTTTAACCAAAGGAGATAATAATGCTGTTGATGACCGAGGTCTCTATAAACAAGGACAACACTGGCTGGAGAAGA | |
| AAGATGTTGTGGGGAGAGCAAGAGGGTTTGTTCCTTACATTGGAATTGTGACGATCCTCATGAATGACTATCCTAAATTT | |
| AAGTATGCAGTACTGTTTCTGCTCGGTTTATTTGTGCTGGTCCATCGTGAGTAAGAAGTCGGACTCCCTGTTCCTAGGAA | |
| GCTGCTGTGCTTGTTGTTACTGAATGTTGGAGTAGATCCTGATCTGTGATTGCGGATTTTCGGAGGACACACACGTTGGC | |
| ACTTCTTGGTAGCCCTGGTTTGCATTGCTTTGTGTTTCCACACCAGAGGCTGTGTGGGCGGGTGCATGTGCACCGTGGAG | |
| TGCACACAAGGGGACTGTCAATCACAGGGTTTCATATGTTGTCATTGTCACTCTTTCACATTTTTGTACATCAGTGAATT | |
| TTTTATATTAAAAGGTTGAGCCAAAGCCCCCAGTGTTTGTATTTTGAAGCCAAGCTTCACTTTAAAAGTGCCTACAGAGT | |
| TCTGTAAATGAAAACACAGCTCTGCATGAGTTCAAACCTGTCGKTCCTYCTTACAGTAGGAATGGCACATATTGAGGCGG | |
| KCATAAGTCTTAACTTTTCAAAATTTTAAATAAAAGACTTTGCACATTTAAAAAAAAAAAAAAAAAAAAAAAAAAA | |
| Clone 0079-Contig | |
| AAAGTTCTTCTCTCACGTGGGTTGGCTGCTTGTGCGCAAACACCCGGCTGTCAAAGAGAAGGGCGGAAAACTGGACATGT | |
| CTGACCTGAAAGCCGAGAAGCTGGTGATGTTCCAGAGGAGGTACTACAAGCCCGGCCTCCTGCTGATGTGCTTCATCCTG | |
| CCCACGCTGGTGCCCTGGKACTGCTGGGGCGAGACTTTTGTAAACAGCCTGTTCGTTAGCACCTTCTTGCGATACACTCT | |
| GGTGCTCAAACGCCACCTGGCTGGTGAACAGTGCCGCGCATCTCTATGGATATCGCCCCTACGACAAGAACATTCAATCC | |
| CGGGAGAAATATCCTGGKTTCCCTGGGTGCCGTGGGCGAGNGGCTTCCACAACTACCACCACACCTTYCCCTTCGACTAC | |
| TCTGCCAGTGAGTACCGCTGGCACATCAACTTCACCACGTTCTTCATCGACTGCATGGCTGCCCTGGGCCTGGCTTACGA | |
| CCGGAAGAAAGTTTCTAARGCTACTGTCTTAGCCAGGATTAAGAGAACTGGAGACGGGAGTCACAAGAGTAGCTGAGCTT | |
| TGGGCTTYTGAGWTCCTGTTTYAAACGKTTTCTGGCAGAGATTTAATATTCTGTTGATTAACTAACAACTGGATATTGCT | |
| ATCGGGGTGTTAATGATGCATTTAACCTATTCCGGKACAGTATTCTTATAAAATGAGAAAGCTTTGATCACGTTTTGAGG | |
| T | |
| Clone 0080-7 Sequence | |
| TCATACAAACACCCCAGTACCTCCAGCAGCTCCAGGAGTCAAAAACACCAGCAGTGGACCGAGGTCCTACAGCTGCCTGC | |
| AGCCATGTTGGCCTTCAGTCTGCTCGTGCTGGGTCTGCTCGCGGAAGTGGCACCTGCCAGCTGTCAACAAGGTCTAGGAA | |
| ACCTTCAGCCCTGGATGCAAGGCCTCATTGCTGTCGCYGTGTTCTTGGTCCTYGTTGCAATCGYCTTYGCMGTCAACCAC | |
| TTCTGGTGCCAGGAGGAGCCGGAGCCTGSGAGCACAGTSMTGAYCRTYGGAAACAAGGCAGATGGRGTCCTGGTGGGAAY | |
| RGATGGMAGRTACTCYTCRATGGCRKCYRGTTTYAAGTCCAGYGAGCAYRAGAATGCCTAYGAGAATGTKCYSGAGGARG | |
| ARGGCARGGTCCGCAGCACMCCSATGTGRCRAGCYKCTMYSYRKMCSWMGTCCCCAGRCTACGCWTAGAGTTGATCCCCA | |
| CCATCTCACCCAGCCACTGCTCTACAGGAATCTACTGAAACAAGCTGACTYCCTCACCTCTCTAGAATCACAGTCATCTY | |
| AGGCCAGGACTCAGACCAAGCCCAGCACACCAATGTGCTGTGACCCYKGGCTTTCC | |
| Clone 0081-Contig | |
| AAAGCGCTCCTCGAGGGTCGGTCCCGCCACGTCTCTTCCCGGTCTCTCTGGCTCTGGAGTGCTTTCCCCCCGCGGAGGGT | |
| GGGAGCGCGGGGACAGACGGGCGAGATGAGCACCATGTTTGCAGACACGCTGCTCATCGTCTTTATCTCCGTGTGCACCG | |
| CGCTGCTCGCCGAGGGCATAACCTGGGTCCTGGTTTACAGGACAGACAAATACAAGAGATTGAAGGCAGAAGTGGAAAAA | |
| CAAAGTAAAAAATTAGAGAAGAAGAAGGAAACTATAACAGAGTCAGCTGGTCGACAACAGAAGAAGAAGATAGAGAGACA | |
| AGAAGAGAAATTAAAGAACAACAATAGAGACCTGTCAATGGTACGGATGAAATCCATGTTCGCGATTGGCTTTTGTTTTA | |
| CTGCCTTAATGGGAATGTTTAATTCCATATTTGATGGTAGAGTGGTGGCAAAGCTCCCCTTCACTCCTCTGTCTTACATC | |
| CAAGGACTATCTCATCGGGAACCTGCTGGGAGATGACACCACGGACTGCTCTTCATCTTCCTGTATATCCTCTGTACCAT | |
| GTCAATTCGACAAAACATCCAGAAGATTCTTGGCCTCGCCCCTTCACGAGCTGCCACCAAGCAGGCTGGTGGATTTCTTG | |
| GCCCACCACCTYCGTCTGGGAAGTTTTCCTGAAGGAAAGCAGAATTCTGAATTTCCTGTCATACTTTTTAGACATTCACA | |
| TCAGACTTACMGAGCACCTGGCCACAWTCTAGGTNKGKGTWATTTCTCAGCTATGGATAGATWCMAATGAGAACCCTGMT | |
| TTAACTAAWGGGAWARTGCTATGGTCACCGGATGGCTTCTTTNAGTAATAAGTGSMCYCNNTCTGGKTACCATTTGGAWG | |
| GCTTAATGTAACCCCMAACCATCAATCTTTCTTGTCTYACTTGAGTATTTCAGTGTATGTGCCATATACACAGAAGCAC [symbol] | |
| CTGTCCATGAACATTAGGAGAAAAATAGTTTATGTAACAACTTCACTGTAATTAGTTGTTGTGTTTTTTAAAAAGCCA [symbol] | |
| TATGTCACTTTGTGTTCGACAATGAGCCAAATTTTTAGATGTTTCAGTTAGAGAAATGTTTGATTTTTTTGCATATTA [symbol] | |
| AAGAGTATTTTCTGTAATCTGAACATAAGGATAAGCATTGGCCCAAAAAGCTAAAATAAAGTTTTGTCATGTTCTTTTAC | |
| CAAAAAAAAAAAAAAAAAAAAAAAA | |
| Clone 0082-Contig | |
| AGGCGACATGGGGAGGCGCGGAGGCGACACCGGAAGTGGCTGTGGTCCGGGTCGGCCCGAGGGGGACTCAGCGCCCGCAG | |
| CAACCACCCGCGCGGCGGCGGTCACAGCTCATACCCGTGCGGGTGGGCGTGGCGGGCGCCGGGACACAGCTCCGACTGTT | |
| CGCTGTCGGCGCGGRGMGKCGCCGCGTCGCTCGCCTTNACCAGCTGCCATGAGCGAGCGCCTCCGCCCCAGAAAAAGGAG | |
| AAGAAATGGCAGTGATGATGACAACCACCCTCCTCCCCAGACCAAAAGGAGCAGTAGGAACCCCATCTTCCAGGACTCCT | |
| GGGACACAGAGTCTTCAAGCAGCGACAGTGGTGGGAGCAGCAGCAGCAGCAGCATCAACAGCCCAGACAGGGCCAGTGGG | |
| CCAGAGAGCAGCCTGAGCCACACCATCCCCGGATCCTGCCCCAGCACCCCCCAGCCGATGCCTGAGCAGTCTGCACTATG | |
| CCAAGGCCCTTACTTCCACATCAACCAGACCCTGAAGGAGGCTCACTTTCATAGCCTACAGCACCGAGGCCGGCCGCCGA | |
| CATGATGCTCCTCCGGCAGTTTCTTGCCTTCTGTGAAGGGACAGCACTGTGCAGATTGGATATTTCAACTTAATGATTTG | |
| TTTTTAAAAGTTGCACACAGAAAAAAAAAAAAAAAAA | |
| Clone 0083-Contig | |
| TGCTGAGGTGGGAGTGGGCCCAGCGCCCGGTGGCCGCAGCTCACGCGCAACCTGCGCCATGGCCGCCTCCGCCGCCTCCT | |
| CCGAGCATTTCGAGAAGCTGCACGAGATCTTCCGCGGCCTCCTTGAAGACTTACAAGGGGTGCCGGAGCGGCTGCTGGGG | |
| ACCGCGGGGACAGAAGAGAAGAAGAAGCTGGTCAGAGATTTTGATGAAAAGCAACAGGAAGCAAATGAAACGTTGGCAGA | |
| GATGGAGGAAGAACTACGATATGCACCCCTGACTTTCCGTAACCCCATGATGTCTAAGCTGCGAAACTACCGGAAGGACC | |
| TTGCTAAACTCCACCGTGAGGTGAGAAGTACACCTCTGACAGCCGCACCTGGAGGCCGAGGAGACCTGAAGTATGGCACC | |
| TATGCCTTGGAGAACGAGCATTTGAATCGACTACAGTCTCAAAGAGCATTACTCCTACAAGGCACTGAAAGCCTGAACCG | |
| GGCTACCCAAAGCATTGAGCGTTCTCATCGGATTGCCACAGAAACTGATCAAATTGGTACAGAAATCATAGAAGAGTTGK | |
| GGGAGCAACGAGACCAGTTGGAACGTACTAAGAGCAGACTGGTAAATACAAATGAAAATTTGAGCAAAAGCCGGAAGATT | |
| CTTCGCTCAATGTCCAGAAAAGTGATAACCAACAAGTTGCTGCTCTCCGTCATCATCTTGCTGGAGCTAGCCATCCTGGT | |
| CGGTCTGGTGTATTACAAATTCTTTCCACACCATTGAACTTCTGTAGGGAAAGCCTTGTGGACCAGCATCTTACTCTGAA | |
| TGAATGGTTTTACATAGGGATATGTGTTGCTGCTGTGGGCTAACAGTTCAAGAATGTATTGTCTTGTCAGACTATGGGAG | |
| GGAGGGCAATGGAAAACCTCGAAAATGTGAAGGAACAGCAACAAGACCAGTATGATATACTAAGGTAATAAATGTTGTTT | |
| ATGACTCCTTCAAACTTAAAAAAAAAAAAAAAAAAAAAAAAA | |
| Clone 0084-Contig | |
| CTTTTTTTTTTTTTTTTTTTTCCGTTATAATAGCCATCTTTATTTGTAAAAATCCAGATATAAAACGTAATCTTTCAGTC | |
| TTTCCAGGTTTTCCTTTTTTACAAAAACAAAAAGGCACGTATAAACCTTGCCCGCTGTCGTCCCCGTACACGGTGTTTCT | |
| CAGGCAGCCCTCCCCCCCGCCCCGCCCCCCGTTACAGCTACATGCTTCATTCCAGCACGTCTGCATCCCCACATGCTTTG | |
| GTGCTTTCCTACCAGGGTAGAGTTCCGAGCTCCAACACTTGAAGTACACAAAGAGGGGGTAGGGGTGGGTGCAGTGTGTG | |
| GCACAATGTTCCACGGCGTGCAGGGCAGTGGGCTAGTACTAGGTCTCCTTCTCCACCCAGCCGCCAGGGCGCCGCCTGAT | |
| AATGAGCTTCCGCACCTCGTCATACACAAAGATGAGAAGGGAGTAGGGGAAGGCACAGAACCACCATGTAGGTTTGAGGG | |
| GATACATCCTAARGGCTGCCCCCATCCCGGGGCAGTAGGATAAGAAAGCAGCAAGGGCTGTCTCTTCAAAGAGGCCAAAT | |
| ATCAAGATCTTGKTCTTCATTCCCTGCTGGAAGACAGAATTCCTTCTGGTCTTGCAGATGACCAAGTCGGCCCACTGCAC | |
| TACCACAATACTGACAAAGAACGCTGTATGGCAGGTGAACTCCACGATCTTCCTCTGCTCGTAGGTCCACTGCTGCCCGT | |
| AGCTGTCCTCCACATCGTTGACCCAGCGGTCATCCCAGGTCTCTCGGATGCCCAACAGGTGAAAGGGCAGGAAACCGTTC | |
| TCAGCCAGAATCACAAAGTAAGTGAAGAAGCCTCCCAGGGCCTGGATCATACCGATCTGTCCATAGGCCATGCTGATCAG | |
| ACGCTCGTTCACAAGTTTGTCCGTTTTGGGGTTTCTGGGCTGCCTCCTTGTGATGTCGCTCTCAGCTTGCTCGTAGGCCA | |
| GGGAGATGGCAGGAACCATGTCAGTGCCCAAGTCAATGCAGAGGATGGTCACAGTCCCCAGGGGCAGTGGAATGTTTGCA | |
| ATAATAAATATCAAGAAGGGGGTGATTTCCGGAATGTTACTGGTTAGGGTGTAAGCGATGGATTTCTTCAAGTTATCAAA | |
| AATCAGACGACCTTCCTCTACTCCAGTCACAATGGAGGCAAAGTTGTCATCCAGAAGGATCATGTCAGCAGCTTGCTTGG | |
| ACACATCAGAGCCAACAATCCCCA | |
| Clone 0085-Contig | |
| AGGTCTGCGGCCCTCGCAGAACTTCCAGCAGCGACATCTTGGGCCAGAGTATCCGGAGGTTCACGACCTCCGTGGTCCGT | |
| CGCAGCCACTATGAGGAGGGTCCGGGGAAGAATTTGCCATTTTCAGTGGAAAACAAGTGGCGGTTGCTGGCTATGATGAC | |
| CGTGTACTTTGGATCTGGGTTTGCCGCACCTTTCTTTATAGTAAGACACCAGCTACTTAAAAAATAAGGATATTTAATTC | |
| ATCCCTTTAACAGAATGAAGAAAGTTTAAGAGGTGATCTGAAAATTGGATTAAACTCTTGAACTCTTATACTAGAAAAAA | |
| TTGTAAATAAACTAATGACATAAAGATTCAAAAAAAAAAAAAAAAAAAAA | |
| Clone 0086-T7 Sequence | |
| AGATGAATTCAAAGAGTGCCCAGGGTCTGGCTGGTCTTCGAAACCTTGGGAACACGTGCTTCATGAACTCAATTCTTCAG | |
| TGCCTGAGCAACACCCGAGAGCTGAGAGATTACTGCCTCCAGAGGCTGTACATGCGGGACCTCGGCCACACCAGCAGCGC | |
| TCACACGGCCCTCATGGAAGAGTTTGCAAAACTAATCCAGACCATATGGACGTCGTCCCCCAATGATGTGGTGAGCCCAT | |
| CTGAGTTCAAGACCCAGATCCAGAGATATGCGCCACGCTTCATGGGCTATAATCAGCAGGATGCTCAGGAATTCCTTCGT | |
| TTCCTTCTGGATGGTCTCCACAATGAGGTGAACCGGGTGGCAGCAAGGCCTAAGGCCAGCCCTGAGACCCTTGATCATCT | |
| CCCTGATGAAGAAAAGGGGCGACAGATGTGGAGGAAGTATCTGGAAAGGGAAGACAGTCGGATTGGGGATCTCTTCGTTG | |
| GGCAGCTGAAGACCTCCCTCACATGCACCGATTGTGGCTACTGCTCTACAGTCTTCGATCCCTTCTGGGATCTCTCGTTG | |
| CCCATCGCAAAGAGAGGTTACCCTGAGGTGACGTTAATGGATTCTATGAGGCTCTTCACCAAASAGGACATATTGGATGG | |
| GTGATGAGAAGCCAACTTGCTGCCGCTGCCGAGCCAGAAAACGATGCATAAAAAAGNTCTCTGTCCAGAGG | |
| Clone 0087-Contig | |
| AGGGCGCCTGAGTGAAAAGTGTGGCACCATGGCCTCTGTGCTGTCCTACGAAAGTCTGGTACACGCCGTGGCCGGAGCCG | |
| TGGGAAGTGTGACTGCCATGACAGTGTTCTTCCCCTTGGATACTGCTAGACTTCGGCTTCAGGTCGATGAGAAAAGAAAG | |
| TCAAAAACGACGCATGCAGTGCTCCTGGAGATAATTAAGGAAGAAGGCCTCCTGGCACCATACCGAGGATCGTTTCCAGY | |
| TATTTCCAGTCTCTGCTGCTCCAATTTTGTCTATTTTTACACTTTTAATAGCCTCAAAGCAGTGTGGGTCAAAGGTCAGC | |
| GTTCTTCTACAGGAAAAGATCTCGTGGTTGGGTTTGTAGCAGGAGTGGTGAATGTGCTGCTGACGACTCCGCTCTGGGTG | |
| GTAAACACCAGACTGAAGCTGCAGGGGGCAAAATTTCGGAATGAAGMCATTATACCARCTAACTACAAAGGCATTATCGA | |
| TGCATTCCACCAGATTATTCGAGATGAAGGGATCTTGGCTCTGTGGAATGGCACCTTCCCCTCCTTGCTGWTGGWCTTCA | |
| ACCCTGCCATCCAATTCATGTTCTATGAAGGCTTAAAACGGCAGCTTCTAAAGAANCGGATGAAGCTCTCTTCTCTGNAT | |
| GTGTTCATCATTGGCGCAATAGCCANAGCGATTGCCNCCNCAGTCACCTATCCCATGCAGACGGTACAGTCAATTCTGAG | |
| GTTTGGACGTCATAGACTGAACCCAGAAAACAGGACCCTGGGAAGTCTTCGGAATGTTCTCTCTCTTCTTCACCAGCGAG | |
| TCAAGCGCTTTGGAATAATCGGACTCTACAAAGGCCTGGAAGCTAAGCTGCTGCAGACAGTGCTCACAGCCGCCCTCATG | |
| TTCCTTGTGTACGAGAAACTGACAGCTGCTACCTTCNCCGTAATGGGCCTGAAGAGCACACATAAGCNCTGAGGCCCGCC | |
| CNCAGGACAGTGTGGAGACGCAGGTGCTGCACAGGGAGAAGAGGAACAGTTCTCCTTTCCCGTTGGTCCCATCCNCNCCN | |
| CAGACATTCCAGTCATTGCCTGTAAAGGCATCAAAGGCTAAGCAGGGAGTATAGCCAGCTAAGCCTGTCACCTTTATGGG | |
| GTTTTTTGGTGTTTTTATGGTGTTTGGTTGTATTTGGGGGTAGAGGTTAGACTGATATGAAAATATTGAAACCCTGAAAA | |
| AAAAAAAAAAA | |
| Clone 0088-Contig | |
| AGTAGAGCCCCTACGGTCATGGCGGCTACCGCCCCCAAGGCCGGGGGTTCAGCTCCTGAGGCAGCGGGTTCTGCCGAAGC | |
| TCCGTTGCAGTACAGCCTTCTTCTTCAGTACCTGGWGGGCGACAAGCGTCAGCCCCGGCTTCTGGAGCCTGGCAGCCTGG | |
| GCGGGATCCCGAGTCCTGCCAAGAGCGAGGAGCAGAAGATGATCGAGAGGGCAATGGAAAGTTGCGCCTTCAAGGCTGWG | |
| CGTTCCTGTGTAGGAGGGTTTGTCTTGGGAGGCGCGTTTGGTATCTTTACTGCTGGCATTGATTCCAACGTAGGCTTTGA | |
| CCCAAAGGACCCTTACCGGACACCAACTGCAAAAGAAGTCCTGAAAGACATGGGACAGAGAGGAATGTCCTATGCCAAAA | |
| ACTTTGCCATTGWGGGCGCCATGTTTTCATGTACTRAGTGTCTGGTAGAGTCTTACCGGGGAAAGTCGGRCTGGAAGAAC | |
| AGCGTCATCAGTGGCTGCATCACTGGCGGAGCCATCGGCTTCCGAGCTGGAGTAAAGGCCGGGGCCATAGGTTGTGGAGG | |
| GTTTGCTGCTTTCTCTGCTGCAATCGATTATTACCTACGGTGAAGGAANCAGCCGGGAAGGANAGAGGACGCCAGCCNCC | |
| TTCANAGCTGCTCTGTGGANCAGTTTCTACACACTTGGACTGCTTCAGGGGTGAAGCCAAGTGCATCCCTCTACAGTTNA | |
| TGGTGTNAAGGACNCNCCTCGCTGCTCGATCGCTCTCTGCAGCCNTTGGAACAACCATACTTTTCCMCTTGAATTCCAAC | |
| CAGGTTTTTCAGGGRAGGTGGTATGGCAGATTGTCTCCTTGCAGTAAMCAGATCAGGAGSCAGTGTGTGGMCTGTTACGC | |
| WTTAAGCTTTGAACCAGGCTCAGTTCTGMCCTGGCTCAGCTCCCCAGCTTTGAATAGGGAAGTGSCAGTGTTTGCCCATA | |
| SCCGGGAAGCTCTTCTTCCTTCTGTGTGCAAGTATCCATCATGAGTCCCAGGGCAGGSCAAAGTAAACTTATGCCATCGA | |
| GGCAAAAAAAAAAAAAAAAAAAAAAAAAAAAAAAAAAA | |
| Clone 0089-T7 Sequence | |
| AAGGCCGGGAGGATGCGGCCGGAGCCCGGAGGCTGCTGCTGCCGCCGCCCGATGCGGGCGAACGGCTGCGTCAAGAACGG | |
| GGAAGTGAGGAACGGGTACTTGAGGAGCAGCACCGCCACCGTCGCGGCTGCCGGCCAGATTCATCATGTAACAGAAAATG | |
| GAGGACTGTACAAAAGACCGTTTAATGAAGCTTTTGAAGAAACACCCATGCTGGYTGCTGTGCTCACATATGTGGGCTAT | |
| GGCGTACTCACCCTCTTTGGATATCTTCGAGATTTCTTGAGGCATTGGAGAATTGAAAAGTGCCACCATGCAACAGAAAG | |
| AGAAGAACAAAAGGACTTTGYGTCCTTGTATCAGGATTTTGAAAACTTCTATACAAGGAACCTCTACATGAGAATCAGAG | |
| ACAACTGGAATCGACCTATCTGTAGTGTGCCTGGAGCCAAGGTGGATATCATGGAGAGAAAATCTCATGACTATAACTGG | |
| TCATTCAAGTACACAGGGAATATAATTAAAGGTGTAATAAACATGGGTTCCTACAACTATCTTGGATTTGCGAGGAACAC | |
| TGGATCATGTCAGGAAGCAGCTGCTGAAGTCCTCAAGGAGTATGGAGCANGGGTGTGCAGCACTCGTCAGGAAATTGGAA | |
| ACCTGGACAAGCATGAAG | |
| Clone 0090-Contig | |
| AGCGGATCTTCGGGCCGGGAGGACATTCGGCCTCTGTGAGCCGCAACCTTGCCCAGCGAGCGGTTGGGNGTTCGCCATCT | |
| TAGGAGGATGTTCTCGTCCGTAGCGCACCTGGCGCGGGCGAACCCCTTCAACGCGCCACACCTGCAGCTGGTGCACGATG | |
| GCCTGTCGGGTCCCCGCAGTCCCCCACCTCCGCCCCGGCGTTCCCGCCACCTGGCCGCCGCCGCCGNGTAAGAGTACAGT | |
| TGNGAATTTGGCTCCATGAAGTATTATGCACTGAGNGGCTTTGGNGGGGTCTTAAGTTGAGGGCTGACACACACTGCTGT | |
| TGTTCCCCTGGACTTAGTAAAGYGCCGCATGCAGGTGGACCCTCAGAAGTACAAAGGCATATTTAATGGATTCTCCATTA | |
| CACTGAAAGAAGATGGCGTTCGNGGKTYGGCTAAAGGATGGGCCCCAACTTTGATTGGCTATTCCATGCAAGGGCTCTGC | |
| AAATTCGGCTTTTATGAAGTCTTCAAAGCCTTATATAGCAACATACTTGGTGAGGAAAACACCTACCTGTGGCGCACATC | |
| ACTGTATTTAGCTTCTTCTGCCAGAGCTGAATTCTTCGCTGACATTGCCCTGGACTCCTATGGAAGCTGCTAAAGTTCGA | |
| ATTCAAAACCCAGCCTGGKTATGCCAACACCTTGAGGGAAGCTGTTCCCAAAATGTATAAAGAGGAAGGCTTAAATGCGT | |
| TCTACAAGGGCGWTGCTCCTCTGTGGATGAGACAGATCCCSTACACCATGATGAAGTTCGCCTGCTTTGAACGTACTGTT | |
| GAAGCTTTGTACAAATTTGTGGTTCCCAAGCCCCGAAGTGAATGTACAAAGGCAGAGCAGCTGGTTGTGACATTTGTGGC | |
| AGGTTACATAGCTGGAGTCTTCTGTGCGATCGTCTCCCNCCCTGCTGACTCTGTGGTCTCTGTGCTGAATAAAGAGAAAG | |
| GCAGCTCCGCGTCTCAGGTCCTGCAGAGGCTGGGCTTCAGAGGTGTGTGGAAGGGGCTCTTCGCCCGAATCATCATGATT | |
| GGCACTCTGACTGCNCTACAGTGGTTCATCTACGACTCTGTCAAGGTCTACTTCAGGCTCCCTCGCCCTCCTCCCCCTGA | |
| GATGCCAGAGTCTCTGAAGAAGAAGCTTGGGTTAACTGAGTAGATACATCAAAGTAACATGGACTGAATCTGCTTGTTGA | |
| TCAGTGTTGAAAGTGCAAAGGAACTTTTATATATTTGACAGTGTAGGAAATGTCTGTTCCTGATATAATTACTGTAGTAC | |
| CCTTGCTTAAAGCAAGAGTTTCAAACTTAATGCTAAATAAACCCATCTATTCCTGAAAAAAAAAAAAAAAA | |
| Clone 0091-Contig | |
| AGGCACTGCTGGCGACATGGCCGACACGGACCCGGGCTATCCCCGCTCGTCCATCGAGGATGACTTCAACTACGGCAGCT | |
| GCGTCGCGTCGGCCAGCGTGCACATCCGCATGGCCTTTCTCAGAAAAGTCTACAGTATCCTCTCTCTGCAAGTCCTCCTG | |
| ACTACAGTCACCTCTGCCCTGTTCCTGTATTTCCAAGCTCTGCGGACATTTGTCCATGAAAGCCCTGCCTTAATTGWGGW | |
| GTTWGCTCTGGGATCTCTGGGCTTGATCTTTGCACTGACTCTGCACAGACACACGCATCCTCTGAACCTCTATCTACTCT | |
| TTGCATTTACACTGTCAGAATCCCTGGCCGWGGCAGCTGTTGWTACCTTCTATGATGTATATCTGGTTCTGCAAGCGTTT | |
| ATAATGACTACTGCAGTCTTTCTTGGCTTGACTGCCTATACTCTACAATCAAAGAGAGATTTCACCAAATTCGGAGCAGG | |
| GTTGWTWGCTGGTWTGWGGATTTTGWGCTTGGCAGGATTCTTGAAGCTGWTTTTTTACAGWGAGACGATGGAGCTGGTCT | |
| TGGCCTCTCTAGGCGCCCTCCTCTTCTGTGGGTTCATCATCTATGATACACACTCGCTGATGCACAGACTCTCTCCCGAA | |
| GAGTACGTGAWCGCTGCCATCAGTCTCTACATGGATATCATCAACCTCTTCCTGCACCTGKTGAAGTTTCTGGAAGCAGT | |
| TAAATAAAAAGTAACCGAGCAGTNGTTCAGAGACAGKTCTATTATGAAAGGACGCTTTGGAATTAAACTTTAAATGGTTA | |
| ATAATTAAAAGCCAWATGTGAWCTTTAAAAAAAAAAAAAAAAAAAA | |
| Clone 0094-Contig | |
| AGGGCCGCAGCAGGCGCGAGCGCCCAGGACCTGCCAGCTGAGCCCTCCGCCGYCGCCATGGGACAGAACGACCTCATGGG | |
| CACGGCCGAGGACTTCGCCGACCAGTTCCTTCGAGTCACCAAGCAGTACCTGCCTCATGTGGCGCGCCTCTGCCTGATCA | |
| GCACCTTCCTGGAGGATGGCATCCGCATGTCGTTCCAGWGGAGTGAGCAGYGTGACTATATCGACACCACCTGGAGCTGT | |
| GGCTACCTGTTGGCCTCATCCTTCGTGTTCCTCAACCTGCTGGGACAATTGACTGGCTGTGTTTTGGTGCTGAGCAGGAA | |
| CTTCGTGCAGTATGCCTGCTTTGGGCTGTTTGGAATCATCGCACTGCAGACAATTGCCTATAGCATTTTGTGGGACTTGA | |
| AGTTTCTCATGAGGAACCTGGCTTTAGGAGGAGGTTTGCTGCTGCTCTTGGCAGAATCCCGCTCAGAAGGGAAGAGCATG | |
| TTTGCTGGTGTCCCAACCATGCGTGAGAGCTCCCCCAAGCAGTACATGCAGCTTGGAGGAAGGGTCCTGCTGGTCCTGAT | |
| GTTCATGACCCTCCTTCACTTTGATGCCAGCTTCTTTTCTATCATCCAGAACATTGTGGGCACAGCTCTGATGATCTTAG | |
| TAGCCATCGGTTTTAAAACCAAGCTGGCAGCTTTGACTCTCGTCGTCTGGCTGTTTGCCATCAACGTCTATTTCAACGCC | |
| TTCTGGACAATCCCGGTATATAAACCCATGCATGACTTCCTGAAATACGACTTCTTCCAGACCATGTCAGTGATTGGAGG | |
| CCTGCTCCTGRTGGTGCCTCTTGGMCCAGCGGGTGTGTCCATGCATGAGAAGAARAAAGAGTGGTAACACACWGATCCCT | |
| CCCCTCCCTGGCTGAGGCRCAGGGCCCTGGCCTGGTTCAGGGCARAGTCAACAAACTGCCCGCGTTTGTGTGTCCTTCTC | |
| CCTTCCCCTCCCTTGGTAAAGGCACAGATGTTTTGAGAACTTTATTTGCAGACACCTGAAAATTGGAGATAAAATCTTTG | |
| GAAAAAAAAAAAAAAAAA | |
| Clone 0095-Contig | |
| AGCTTCTTCAGTCGGGACGGGGTCAGCGAGCGGCTGCTTCGTGGAGCAGAGAGGTGCATCACCAGGTTCCCGATGAACCC | |
| AGAGAACCCTCCACCGTATCCGGGCCCCGGGCCAACAGCCCCATACCCACCTTATCCACAACAGCCAAWGGGGCCAATGG | |
| GGCCTATGGGAGCCCCACCTCCTCAGGGGTACCCCTACCCACCACCTCAGGGGTACCCCTATCAAGGATACCCACAGTAC | |
| GGCTGGCAGGGTGGACCTCAGGAGCCTCCTAAGACCACAGTGTATGTGGTGGAAGACCAAAGAAGAGACGACCTGGGCCC | |
| ATCCACCTGCCTCACAGCCTGCTGGACTGCTCTGTGTTGCTGCTGCCTCTGGGACATGCTCACCTGATCACCTGATGAGC | |
| CCAGCTCTTCCGCTTGGCCGCTCTGTGCCACCTCCGATAAGTGTGCCTGGCCCCATCTCTTCTGATTGCTATAAAGTGGC | |
| TAGCTCTGCGCAGACACCTCTACTTTCTGTCCTATGGAGTTTTAGATTAGTGAGGGTTACTGCTATTTAGTTCAGTGACT | |
| TGATCTTTTTAATGTTCAAAACTCATTTCTTACTGATCTTTAAAAAATGTGCTAAATAATTTACTTTTTTGGCCAAAGGC | |
| TTAGTTATGAAATATATATTTATAATGTTTAAATTATACACTCAAGGTAATGGCAATATGTGACACACTACTAGATCTCT | |
| CTGCTCATACCCTTTATGTTTCAATAAATCTGTCCAAAATCAAAAAAAAAAAAAAAAAAAA | |
| Clone 0097-T7 Sequence | |
| CTTTCCAACAAGGACGACATGAAGACCTCACTCAAGAAAGTTGTGAAGGAGACATCGTATGAGATGATGATGCAGTGTGT | |
| ATCGCGAATGCTGGCCCATCCCTTACACGTGATCTCGATGCGATGCATGGTGCAGTTTGTGCGACGGGAGGCCAAGTACA | |
| GWGGTGTGCTGAGTTCTATTGGGAAGATCTTCAAGGAAGAGGGGCTGCTGGGATTCTTCGTTGGCTTAATCCCTCACCTC | |
| CTGGGCGATGWGGTTTTCTTGTGGGGCTGTAACCTGCTGGCCCACTTCATCAATGCCTACTTGGTGGACGACAGCGTGAG | |
| TGACACCCCAGGGGGGCTGGGAAACGACCAGAATCCAGGTTCCCAGTTTAGCCAGGCCCTGGCCATCCGGAGCTACACCA | |
| AGTTTGYGATGGGGATTGCAGYGAGCATGCTGACCTACCCCCTTYCTGCTCGCTGGAGATCTCATGGCAGTGAACAACTG | |
| TGGGCTGCGGGCTGGACTCCCTCCGTATTCCCCTGTGTTCAAGTCCTGGATCCACTGCTGGAAGTACCTGAGTCTGCAGG | |
| GCCAGCTCTTCCGCGGCTCCAGCCTGCTTTTCCGCCGGGTGTCATCGGGGTCATGCTTTGCCCTGGAGTAACCTAAGCTG | |
| CCCGACCAAACATTTATGGGGTCTTAGCCTACCCCTGGTGAGGACCCATCATCTCAGATGCCCCAAGGGTGACTCCAGCC | |
| CAGCCCTGGCTTCATGTCCATATTTGCCATGTGTCTGTCCAGATGT | |
| Clone 0097-BGH Sequence | |
| CTTTTTTTTTTTTTTTTTTTCACCACCAATACATTTATTCGAGGAGATGGGTCTATCTTACCACGAGGGGAGGACTAGAT | |
| CTCGGYCTATGTAACCTGTGCGTATTCGCACCCAGCACAGTGACTGAACCCTCACACCTGGCGTCACCAGCACAGACAAG | |
| CAGATGAGGGGATGGTCTGAGGAGAACATGATTTCCTATTCAGGAGAAGGCACCACCCTTGTATAAGAAAATTAGTGTTG | |
| GGAACATAGCGCCAGCCTCCCATGGCCCAGGTGTGATGGCGCCCAATTTACAAGCAAGGAAGTGGGGGGCGGSGGTGCTT | |
| CTGGCTGACTGGCAGGATGAGCTGGGCTAGAGGTGCAGGGAAGCCTTGCCACTGAGTGACGTTTGCCTCTGCAGCCTGCC | |
| TCTGCCTGAGTACAAGATGGACTCCAGTACCTCTAGGCAGGAAGGGGATGCCACCCCACCACTGCTC | |
| Clone 0098-T7 Sequence | |
| AGGGCGGAGGAAGCGGACTGTTCCGGAGCTCTGCCTAGCCGGGCCCAACCTTTGCTCCAGAGATCATGGCTGCCGAGGAT | |
| GTGGTGGCGACTGGCGCCGACCCTGAGCGAGCTAGAGGGCGGCGGGCTGCTGCACGAGATTTTCACGTCCTCCTCTCAAC | |
| CCTGCTCCTCCTGGGCCTCTGCATCTTCCTGCTCTACAAGATCGTTCCGCCGGGACCAGCCCGGTGCYAGTGSCGACAAC | |
| GACGAYGACGAACCACCCCCGCTGCCCCGCCTCAAGCGGCGCGACTTCACCCCTGCCGAGCTGAGGCGTTTCGATGGCGT | |
| CCAGGACCCGCGCATTCTCATGGCCATCAACGGYAAGGTGTTCGACGTGACCAAAGGCCGCAAGTTCTACGGGCCTGAGG | |
| GGCCATATGGGGTCTTTGCCGGAAGAGATGCATCCAGGGGCCTTGCCACATTTTGCCTGGACAAAGAAGCACTGAAGGAT | |
| GAGTATGACGACCTTTCTGACCTCACCCCTGCACAGCAGGAGACCCTGAGTGACTGGGACTCTCAGTTCACTTTCAAGTA | |
| TCATCACGTGGGAAAACTGCTGAAGGAAGGGGAGGAGCCTACTGTGTACTCAGATGATGAAGAACCAAAAGAYGAGACAG | |
| CTCGGAAGAATGAATGAAGCATTCGGTGGAGCATATCTATTTTTGTATTTTGCAAAATCATTTGTAACATTCCAGTCTGT | |
| CTTTACAACATGGTGATTTCAATATTTAGAAAAGNTTTGAACTTGCTGTAAATTTTTTAAGTAACATCACTAGTGACACC | |
| GATAAAATCAACTCG | |
| Clone 0099-Contig | |
| AGCTCGAAAGCGACATGGCGGTTCTCTTAAAGCTGGGCGTTCTCTGCAGTGGCCAAGGAGCTCGAGCTCTCCTACTCCGA | |
| AGCCGGGTGGTCAGACCCGCTTATGTGTCAGCATTTCTCCAGGACCAGCCTACCCAAGGACGGTGTGGTACCCAGCACAT | |
| TCACCTGTCACCAAGCCACCACTCTGGTTCCAAGGCTGCATCTCTCCACTGGACCAGTGAGAGGGTTGTCAGTGTTCTGC | |
| TCTTGGGGCTGATCCCTGCTGGGTACTTGAATCCCTGCTCTGTGGTGGACTACTCTCTGGCTGCAGCCCTCACCCTGCAC | |
| AGTCACTGGGGCCTTGGACAAGTGGTTACCGACTACGTTCATGGGGACACCCTGCCGAAGGCTGCCAGGGCAGGCCTCTT | |
| GGCACTCTCAGCTTTGACCTTTGCTGGCCTTTGCTACTTCAATTACCACGATGTCGGCATCTGCAGAGCGGTTGCCATGC | |
| TGTGGAAGCTCTGACCTGGGTGCAGCACTTTGATTGTGTGCCTCCTTGCCTCTGCTTTACCAATGCCGTTCACCTCGCAG | |
| TGAGGGGGGATGAAGGATAAGCCCATTGGTGGGCAGAATGTCTTCTAATTACATGGTTATTTTCAGAATTTATTTGTTGA | |
| GGAAGAGGTTTGAGGAGTTAGGTTCGACCATTCGTGAGTCTGTGTTCCATACTCCACTGAGTGTGGGCACTAGCTCACAG | |
| CCTCGCGGTGAGACTGAACATTTCATGAGCTCATGKTGCCTTTGACCACCATTTTCTTAAGGAGAGCCAGCTGATTGCTG | |
| TCAGGATAAGAGCATCTCTTCAGCCAGGAGGGAGGCCTGTTCCCTCCTGAGTTAGACTTTGCATGAAGCTCGAAAGTATT | |
| CCCTTTGGAACCTCCCATTCTTGTTCAGGTGACACCAGCTCTGTTGATGGCTCTGCTTCTAGGGAACATTTAATCAGGAG | |
| ATGCTCTCAATGACTAATTTGTCTAAGTCTTAGGAAGGAGGTTGAGGAAAGCTGGATTTAGACAAGTTCAATTTAGGGAG | |
| TTCTCCTTGTTTGTGGATTAAAATATGACAGATTGCAAACAGACTACTCTTCAAATGTATCTCAATTGTGCAGAAGTGAG | |
| CTGTCCAAAAGTATAAGACTAAGTGATAAACTGTCTTCCCACCGTGGGAGTTGTTAATGAGAAAGAAAGTGTACTCTGAA | |
| AAAACAAAATTTTTAAATAAAATATTATATAATGAAAAAAAAAAAAAAAAAA | |
| Clone 0100-Contig | |
| AGCTAGGATCTTTAGTCTTCAACTCCTACTGCTCCTTCTAACCCAGCAGCCCCGGATAATGCAGCCCAGGAGGAGCTCAT | |
| GATCACCCTGATCACAGGATTGGCGTCCCTCACGTCGAGAACCTCCATGGGCATCATCGKTGKTGGGGGCCGGTRATTTG | |
| GAAAACAGGTGCGCTGGAAACTAATCTCTGTCACCTTAAGTATGTACGGAGCTCTGTACCTTTATGAGAGGCTGACGTGG | |
| ACGACCCGTGCGAAAGAGAGAGCGTTTAAGCAGCAGTTTGTAAACTATGCAACTGAGAAGCTGCAGATGATTGTGAGCTT | |
| CACCAGTGCAAACTGCAGCCACCAAGTACAGCAAGAAATGGCCACTACTTTTGCTCGACTGTGCCAACAAGTCTGATGTT | |
| ACTCAGAAACATCTGGAAGAGGAAATTGCAAGATTATCCAAAGAGATAGACCAACTGGAGAAAATACAGAACAACTCAAA | |
| GCTCTTAAGAAATAAAGCTATTCAACTCTGAAAGTGAGCTGGAGAATTTTTSGAAGCAGTTTCTACACCCGAGCAGTGGA | |
| GAATCCTAACGGCAGAGGCACTGTAGGAGGAAGCGGACTTGGAAGATGGGAAATGTTACTTTATGAAATGACCTCAGTAC | |
| AAATTACTAACTCTTAGTATCGATGCCTTGCGGAGATTGTGGTAATGACCTGTCTCAGGGGTTGCACCTTTGGAAGTGTT | |
| GTGATTCGCCTTGTCTTAGCATTAGTTTGGAGTAAAGACTGAATTGTTAAGGTTAAATGATGAATTCCTTTAGAAACAGT | |
| GGAACCGGCTGTKCGGCCCCTGAGGGTGGGTCCTGCAGCTCCTCACCAGGCTGGCTGTCTGCGGCTCTCAGAAGCTGCTT | |
| CCTGGCATCCAGGAGTTAGAGACCTTTTCATCCTTTCTCAGTGCTAGTTCTTGATGCTTCTTTAATGGGAATAGTGAACT | |
| TGTTTATAASCCGATTTGCTCAAACGAGGGGTGTGGGCTGCTCCTGGGGGGGTCCTGCAATCACTCTGTCCTCACAGCAA | |
| GGATGTAACCMCTACTAAACAGTTTTTACTTTCTTTTATTCCCATTAAAGCTGATGTGAAATAGTAAAAAAAAAAAAAAA | |
| AAAAAAAA | |
| Clone 0101-T7 Sequence | |
| AGTCTGCGCCCCTGTACCCTAGGTCTAGTAGTGAACCCGCACCCGRKCAAAGAAGTCTAGGCCGGRGTCCGCAGMATCAG | |
| CCATCACTTCGCACAAAGCAGCGCAGCTCCGGGACCGCCGAGGACCACAGCGGCGCGGCAGCGGYGCGGMGACACTCAGT | |
| GCACCGTATGCCCCTGCGMCCCTGCCGAGGCAACCGGAGCGCCCCGAGAGACCGCGCCGCCGCGGCGWCCAGGTGCAGYT | |
| AGCGAGCCTAGCCCGCAGCGCGCAGTCGCGGGAGAGCGGGGAGCGGCAAGCAACAGGGAGCGGGACGGCGGCGAGGCGCT | |
| CGCGGGCCCCTCCTGCTGCCCGCGCCCGGCGAGCTCATGGCGGCCATCCGCAAGAAGCTGGNGGNGGNGGGCGACGGCGC | |
| GTGCGGCAAGACGTGCCTGCTGATCGTGTTCAGTAAAGACGAATTCCCCGAGGTGTACGTGCCCACCGTGTTCGAGAACT | |
| ATGTGGCGGACATCGAGGTGGACGGCAAGCAGGTGGACCTGGCGCTGNGGGACACGGCAGGCCAGGAGGACTACGATCGT | |
| TTACGGCCGCTCTCCTATCCGGACACCGACGTCATCCTTATGTGCTTCTCGGTGGACAGCCCGGACTCTCTCGAGAACAT | |
| CCCCGAGAAGTGGGTGCCCGAGGTAAAGCACTTCTGCCCCAATGTGCCCATCATCCTGGTGGCCAACAAAAAAGACCTGN | |
| TCAGCGACNAGCATGTCCGACGGAGCTGGCCCGCATCAAGCACCAGCCAGTNCNGCACGGATTCACGG | |
| Clone 0102-Contig | |
| AGGTTTCCTCGCCGCCGGCCAAGATGAACCGATTCTTCGGAAAAGCGAAACCCAAGGCTCCGCCACCTAGCTTGACGGAC | |
| TGCATTGGGACGGTGGATAGCAGGGCAGAATCCATTGACAAAAAGATTTCCCGGCTGGATGCTGAACTAGTGAAATATAA | |
| GGATCAAATCAAGAAGATGAGAGAGGGTCCTGCTAAGAACATGGTCAAACAGAAAGCCCTGAGAGTTTTAAAGCAAAAGC | |
| GGATGTATGAGCAACAGCGAGACAACCTGGCCCAACAGTCCTTTAACATGGAGCAAGCTAATTACACCATCCAGTCACTA | |
| AAGGACACCAAGACCACGGTTGATGCCATGAAGTTGGGAGTAAAGGAAATGAAGAAGGCATATAAGGAAGTAAAAATTGA | |
| CCAGATTGAGGACTTACAAGACCAGCTGGAGGATATGATGGAAGATGCAAATGAGATCCAGGAAGCCCTGGGCCGCAGCT | |
| ACGGCACCCCAGAGTTAGATGAGGACGACCTGGAAGCAGAGTTAGATGCGCTGGGCGATGAGCTTCTGGCTGATGAAGAT | |
| AGCTCCTACTTGGATGAGGCAGCTTCCGCTCCTGCAATTCCGGAAGGTGTTCCCACTGACACAAAAAACAAGGATGGCCG | |
| TGCTGNNGGATGAATTTGNACTGCCGCAGATTCCCGCTTCGTAGACTTACAACATTCAGCACGTGATGTGAAACAACAAG | |
| AGAAGTATTCTGGGACTAGGAAATAGTTCCCCGATCTGCCAACCAGATTTAGGTTTCTTTCCTTTCTTTGAWGAAAAAGC | |
| TNTNTACACTGCTCCNGNATTTTTATTTTTTCCGNTAAGGAATTCATTGCTTTTGGGAAACTGTCTTTACTAAAACTTGA | |
| TTCNTTTTTTTTTTTTTCTCTTAGGAAAGACTAATTGAAAAGTACCCTTGACTTTGTATGACTTGTTTTCATTCATTAAC | |
| AATAATCTGAAATTAAACCAAGGAGATGAGACTCTGAATTCTATGGTAGNGTAAGTACAGTCTCAGTGNGCTGATACATT | |
| GATAAGATAAAAGTGATTGATGAGATTGGGACTGCTGATAGTATGCTTCAGAACCCTTGTCTGTTGTGGTATTGTANATG | |
| GGTTTAAGTCATGGCCTCTTTTGATANATTTTGTTGTGTCATGTGAGCAAGTCATTNCACGATNTACTGTTGGAATGAAC | |
| TGTCTCTTCGGTATCATGAGTTACTATTTTGATTCCATGGTTCCCTCCAGTATACTAGCCTGRCTTGTAATGAATAATGA | |
| ATATTTCTTGATATTTAATGKATAGGRCMTTTATTTATACTCAATWAATATTTTTCMAAAGGAAAAAAAAAAAAAAAAAA | |
| AAAAAAAAAAAAAAAA | |
| Clone 0101-Contig | |
| AGGGCGGCGGCCTTGGGCCGCGGGGAATGGGAGTCCCAGGGCCCTGACTGAGCACCGCCCCCGCCTCCCCGCCGCGCCAT | |
| GGCTCAGGAGAAGATGGAGCTGGACCTGGAGTTGCCCGCGGGCGCGAGCCCGGCGGAGGGCGGCGGCCCTGGCGGCGGGG | |
| GCCTGCGGAGGTCTAACAGCGCCCCCCTGATCCACGGCCTCAGCCACTCGTCCCCCCTGTTCCAGGCCGACGCGCCCAGC | |
| GCCAGACGGAACAGCACGACGTTCCCGAGCCGCCACGGCCTGCTGCTCCCGGCCTCGCCGGTCCGAATGCACAGCAGCCG | |
| CCTGCACCAGATCAAACAGCAGGAGGCCATCGACCTCATCAACCGAGAGACCGTTCACGAGCGCGAGGTGCAGACCGCAA | |
| TGCAGATAAGCCACTCCTGGGAGGAAAGTTTCAGCCTGAGTGACAACGACGTCGAGAAATCCGCCTCCCCGAAGCGCATC | |
| GATTTCATTCCGGTGTCTCCGGCACCGTCCCCGACCCGGGGAATTGGGAAGCAGTGTTTCTCACCGTCCTTGCAAAGTTT | |
| TGTGAGTAGCAACGGACTGCCTCCAAGTCCCATTCCCAGCCCAACCACCCGATTTACCACCCGGAGAACCCAGAGTCCCA | |
| TCAACTGCATTAGACCAAGTGTTCTTGGACCATTGAAAAGAAAATGTGAAATGGAAACTGATTATCAGCCAAAGAGATTT | |
| TTCCAGCGCATCACCAACATGCTGTCTTCTGACGTTGCACAGCTGTCAGACCCCGGTGTGTGCGTATCTTCCGATACCCT | |
| GGATGGAAACAGCAGCAGTGCCGGATCTTCCTGTAACTCACCAGCCAAAGTCAGCACTACCACCGACTCTCCTGTGTCGC | |
| CCGCCCAAGCAGCCTCCCCCTTTATTCCAGTAGATGAACTTTCCTCAAAGTGACTCACTCATCTTGAATCCCCCTCCCCC | |
| AATGGAGAGACAAAAAAACAAAAACAAACAAAAAAAAAAAAAAA | |
| Clone 0111-Contig | |
| AGGCAATGGCGGACGTGTCTGAGAGGACGCTCCAGGTGTCCGTGCTAGTGGCTTTCGCCTCTGGAGTGGTCCTGGGCTGG | |
| CAACCCAATCCCCTGCCGAGGCGTTACCTAGACTGGAGGAAGCGGAGGCTGCAGGACAAGCTGGCAACGACTCAGAAAAA | |
| GCTGGACCTGGCCTGAGCACGCGCTGCAGCCCGAGTCCGCCGGCTTCTCACTCCCTAAGCCCAACGCACCCCCGATCGTG | |
| GGAGCCGCGCGACCCAGGAGTCGTCCTTGCACGGCTTGCAAGAACATGGCTTGCTTCAGAAAGAAAATAGTTTTGTCTTC | |
| TCTAACAACTTACTTTCAGCTTGTCGAAGATGAAAATAAAAAGCACTGGAGAGAAATAATTTCTTGCACTTTATGAATCT | |
| ATTTTTAAAATAAAAAAATTAAMCATCAAAAAAAAAAAAAAAAAAAAAAAAAAA | |
| Clone 0112-Contig | |
| AGGGACTTTGTTCAGATCTTTTTCTGATCGTGCATCAGCTGCTGGTTCATTATAGCCTTCTCTTAAGCACTGAATAAGAC | |
| AAAAAATGAATCTTGGAAGAACAAATTCAGACATCATCAGTAAGTCTTTGGGGACACAGGGAATATTTGAACTTGATTTA | |
| ATCTGATGTCTTCTACAAAACCCGCTCTCCCGCATTACGTTGCTGTCCCCGCAGTCGCAGGCGCCCCCCGCCTGCCTGCG | |
| GAACATGTTCAAGTCGTGTCCAGTGTGGTCGCCCTGGTGGAAGCACTCCGCGCACAGCGACATGCAGGGGGAGATGCCGC | |
| ACGTCCGGCAGCGGTAGGCCACGAAGTTGGCCGTCCAGACCACGCCCCAGAGCGCCGCGGGGTCGTAGGCCCGCACGGCC | |
| GCGCAGAACTCCTCGTAGCCGCCGCCGCCGGCCAGAAGGCACTTACACCACTCCAGGGCATCCTCCTCCGCCTCGCCCGG | |
| GCCGCCGCCGCCGCCCCCCCCCGCCTCCTCGCCGCCCGCAGCCCCGCCCAGC | |
| Clone 0113-T7 Sequence | |
| AGGCGACGCGCGCATGGAGGCCGGCTGAGGAGCGCCGCCGCCTCTCTCGGTAAGGACTGTGTCTGTGTCCCCAGGCATCC | |
| TACATCAATCAGGAAGCTGCTGTCCAGCCATGGAGGGAGAGGAGAAGCCAGCTCAAGAGGCTGACGTGGAACCTGTGGTA | |
| ACAGCAGGCACCTCAGAAGCAGTGCCAAGGGTGCTTGCTGGAGACCCTCAGAACATCTCTGATGTGGATGCCTTCAACTT | |
| GCTCCTGGAGATGAAACTGAAACGACGGCGTGAACGCCCCAACCTTCCACGTACAGTGACCCAGCTAGTGGCCGAGGATG | |
| GGAGCAGGGTGTATGTGGTGGGCACTGCTCACTTCAGTGATGATAGCAAGAGAGATGTAGTAAAGACTATCCGGGAGGTG | |
| CAACCGGATGTGGTCGTGGNGGAGCTCTGTCAGTACCGGGGTGTCCATGCTCAAGATGGACGAGAGGACGCTGCTGCGAG | |
| AGGCCATGGAGGTCAGCCTGGAGAAGCTGCAGCAGGCTGTCAGGCAGAATGGNTTATGTCTGGACTCATCAGATGTTGCT | |
| GCNTGNAGGTGTCTGCTCACATCACTGAGCAGCTGGGCATGGCCCCTGGTGNCCAGTTCAGGGAGGCCTTCAAAGAGGCC | |
| AGCAAGGTACCATTCTGCAAATTCCACCTGGGNGACCGACCAATCCCAGNC | |
| Clone 0115-T7 Sequence | |
| AGCCCGTCCCCAGCGCTCCGGCGAGCTGGCCTCCGCTGCAGTTGAGCCGGGCTCGCACTATGCAGCGCCTGGCCATGGAC | |
| CTGCGGGTGCTGTCCCGGGAGCTGGCGCTCTACCTGGAGCACCAGGTTCGCGTCCGCTTCTTCGGCTCGGGCGTGGGCTT | |
| GTCACTGATCTTGGGATTCAGCGTTGCCTATGCTTGCTACTACCTGAGTAGCATTGCCAAGAAACCCCAGTTAGTGATTG | |
| GAGGGGAGAGTTTCAGCNCGCTTCCTACAGGACCACTGTCCCGTGGTGACAGAAACCTATTACCCGACGGTCTGGTGCTG | |
| GGAGAGCCGAGGACAGACATTGCTGAGACCCTTCATCACTTCCAAACCCCCGGTGCAATACAGGAACGAACTCATTAAAA | |
| CTGCAGACGGAGGACAGATCTCACTGGACTGGTTTGATAACAATAACAGTGCGTACTATGNGGATGCCAGCACCAGACCN | |
| TACTATCTTGCTCTTGCCTGGCCTNACTGNAACAAGCAAGGAATCCTACATCCTTCATATGATCCATCTCAGTGAAGAAT | |
| TNGGATACAGGTGNGNGGNTTTTAATAACANAGGAGTAGCANGAGAAAGTCTCTTGACACCACCGGACTTACTGCTGNGC | |
| GAACACTGAAGAACTTGGAGGCCGNNGTCCACCACGTGCACAGCCTGT | |
| Clone 0116-Contig | |
| AGGGCGCAGCCGGCCTAGCGAGGTCAACATGCCGGTCGCCAGAAGCTGGGTTTGTCGCAAAACCTATGTGACCCCACGGA | |
| GACCCTTCGAGAAGTCGCGTCTCGACCAGGAGCTAAAGTTGATTGGAGAGTATGGACTCCGGAACAAACGTGAGGTTTGG | |
| AGGGTCAAGTTTACCCTGGCCAAGATCCGTAAGGCGGCCCGGGAGCTGTTGACGCTGGACGAGAAGGATCCCCGGCGTCT | |
| GTTTGAAGGCAATGCTCTCCTGCGGCGGCTTGTTCGCATTGGGGTGCTGGACGAGGGCAAGATGAAGCTGGATTACATCC | |
| TGGGACTGAAGATTGAGGATTTCTTGGAGAGGCGGCTGCAGACCCAGGTCTTTAAGCTGGGCCTGGCCAAATCTATTCAC | |
| CATGCCCGTGTGCTCATCCGCCAACGTCACATTAGGGTCCGCAAGCAGGTGGTGAACATCCCATCCTTTATTGTTCGCCT | |
| GGACTCGCAGAAGCACATCGACTTCTCCCTCCGTTCTCCTTATGGTGGCGGCCGTCCAGGCCGAGTGAAGAGGAAGAATG | |
| CCAAGAAAGGCCAGGGCGGGGCTGGAGCTGGTGATGATGAGGAAGAGGATTAATTCTTGGCTGAACTGGAGGATTGTCTA | |
| GTTTTCCAGCTGAAAAATAAAAAAGAATTGATACTTGGAAAAAAAAAAAAAAAAAAAA | |
| Clone 0117-T7 Sequence | |
| AGCGGCGGCTCTGAGTCGCCGGAGCCGCCCCGGGACATCATGGCGGAGGTCTGCGAGCCCACCCGCCCCTCGGAGGACGA | |
| GGACGAGGAGCGGGAGCCGCTGCTGCCTCGCGTTGCCTGGGCCCAGCCGCGGAGGGTCGCGCCCGGGAGCGCCGTGAGAA | |
| TGCAGGCGGACGAGGGCGCGGATGTCCTCCGCGAGCCCGCTACCGACGAGCCGCCGGCGGTGTCCGGGGAAGGGTCGATC | |
| TCCGCGAGCTTGTCCACCGAGCTGGATCGGACCCGCACTACGAGCTCAGAAACAAATACATTCTTGGAAGATCCAGAATT | |
| TGCTGATATTGTATTGAAAGCAGAGCAAGCAATAGAAATTGGAGTTTTCCCAGAAAGAATTTCT | |
| Clone 0119-Contig | |
| CATCATCGAGACAAGCACCATTAAGCCTTACCGTCGAGGGTTTTACTGCAATGACGAGAGCATCAAGTATCCCCTGAAAG | |
| TCAGTGAGACTATAAACGATGCTGNGCTCTGNGCGGNGGGGATCGTCATCGCCATCCTGGCGATCATTACAGGGGAATTC | |
| TACCGGATCTATTACCTCAAGGAGAAGTCCCGCTCCACCACTCAGAACCCGTATGTGGCAGCNCNCNATAAGCAAGTGGN | |
| ATGCNTCCTTTNNGNTGTGCCAATTAGCAAGTCCTTCANNGNCATCGCCAAAGTGTCCATCGGGCGCCTAAGGCCTCACT | |
| TCCTNAGCGTCTGTGACCCTGATTTCAGTCAGATCAATTGCTCCGAGGGCTACATTCAGANCTACAGGTGCAGAGGAGAA | |
| GNCAGCANAGTACAGGAGGCCAGGAAGTCCTTCTTCTCGGGCCACGCCTCCTTCTCCATGTTCACTATGCTGTATCTGGT | |
| GCTCTACCTTCAGGCCCGCTTCACCTGGCGCGGGGCCCGACTGCTCCGCCCCCTCCTGCAGTTCACTTTGCTCATGATGG | |
| CCTTCTACACGGGATTGTCACGGGTATCTGACTACAAGCATCATCCTAGCGATGTCCTGGCAGGATTTGCCCAAGGAGCT | |
| CTGGTGGCCTGCTGCATAGTGTTCTTCGTGTCCGACCTCTTCAAGACTAAGACGAGCCTCTGACTGCCCGCCCCTGCGAT | |
| CAGGAGGGAGATCCTGTCTCCCGTGGACATCATCGACAGGAACAATCACCATAACATGGTGTAGATGCTGCGGCCTCCGG | |
| AGCGCTTTCTCTGAAGCGACTGCACGTTCCTGCTGCTCTCCGATCTCATCAGACAGTAGAATGTAGGGAAAAGCTTTTGC | |
| CCGATGGATTTTGAAAACATTTAAAAAAAAAAAAAAAAA | |
| Clone 0121-BGH Sequence | |
| TGCAGGGAAGACAGAAAGGCCTCCCACGCCACTTGGTTTATTAGATCCTGAAGAGAGGTGTAGGCAGTGCCCCTGGGCCG | |
| CTGCCACCTCCTGGGGGAGGACCTGTGGGAGGCACAGGGCCGAACCTCGTTTTGATACACACAACCCCATTTGAGGGAAA | |
| ACAGGCTGCTTCGAAGCCTGAGGGATGGTGAGGGGTGATGCCTGCCATACAGGAAGCCAGGTCCTGGGAGGGCACAAACG | |
| ATGAATCCATCACTGCCCCAGCTCTGCCAGCATGCCCACCTGGCCCTGGGGAAGCCAGGCAAGGGAGGGCACAGGCGTGT | |
| GAGGGACACAGACAGTTCCTGGTGACGGCAGTAGCTGCTGAGCAGGAGGGTTCAGCAAAACTGACCATTAGAGCAGCCAA | |
| GGCTGCATATAGGAGGTGCGCTCGGGAACCCCAGGCACTTTCTCTGGACTCCACGGTCATGGCTTCTGCTGGTGATCTGC | |
| ACTGCCTGCCTGTCCCCTCTCCTGAAGGCACTACCTTCCAGAACACAGCACGGTGGTCCCTCTTGTGACAAAGTGCTTGT | |
| GTGTGTGCTCTAGTCATTTGGTAAGCAGAAGCTGCCACGGGCCATACCCTGCCACACTACCCAAGTTCTGGGCNGGAAAC | |
| TGCTCCCTGCACAGAGGGCCAGCGGGAGCAGGAAACGAACTCAACTTCGCTGGGCTTGCCAGGCANGGCACGGTAGCAGC | |
| CAGGGAGGTTGGGACAGTGACANCAGNCAGGCAGACTTTTTTG | |
| Clone 0123-T7 Sequence | |
| AGCAACCGCAACCTGACCTTGACCAAGAAGGAACCTGTTGGGGTCTGTGGTATTGTCATCCCCTGGAACTATCCCTTAAT | |
| GATGCTGTCCTGGAAGACAGCAGCCTGCCTGGCTGCCGGGAACACCGTGKTGATCAAGCCTGCCCAGGTGACCCCACTCA | |
| CAGCCTTGAAGTTTGCAGAGCTGACACTGAAGGCTGGCATTCCCAAGGGTGTGGTCAACATCCTCCCAGGATCTGGCTCG | |
| CTGGTTGGCCAGAGACTCTCAGACCACCCTGATGTGAGGAAAATAGGGTTCACAGGCTCCACGGAGGTGGGAAAACACAT | |
| CATGAAAAGCTGTGCCCTGAGTAATGTGAAGAAGGTCTCCCTGGAGCTGGGTGGAAAGTCACCCCTTATCATCTTTGCTG | |
| ACTGTGACCTCAACAAAGCTG | |
| Clone 0124-Contig | |
| TGGAAAGGCGATGGGCATCTCTCGGGACAACTGGCACAAGCGCCGCAAGACCGGGGGTAAGCGAAAACCCTACCACAAGA | |
| AGCGAAAGTATGAGCTGGGACGGCCCGCTGCCAACACGAAGATTGGCCCTCGCCGCATACACACAGTCCGAGTTCGAGGA | |
| GGCAATAAGAAGTACCGTGCCCTGAGATTGGATGTGGGGAACTTTTCCTGGGGCTCTGAGTGTTGTACTCGCAAAACAAG | |
| GATCATTGATGTTGTCTACAATGCATCCAACAACGAGCTTGTCCGCACCAAGACCCTGGTGAAGAACTGCATCGTGCTTA | |
| TTGACAGCACGCCGTACCGACAGTGGTACGAGTCCCACTATGCACTGCCCCTGGGCCGCAAGAAGGGGGCCAAGCTGACT | |
| CCTGAGGAGGAAGAGATTTTWAACAAAAAACGATCAAAGAAAATTCAAAAGAAATWCGATGAAAGGAAAAAGAATGCCAA | |
| AATCAGCAGCCTCCTGGAGGAGCAGTTCCAGCAGGGCAAGCTTYTAGCCTGTATTGCCTCMCGACCAGGCCAGTGTGGCA | |
| GAGCAGACGGCTATGTGCTCGAAGGCAAGGAGCTGGAGTTCTATYTGCGGAAGATCAAAGCCCGGAAAGGCMAATGAGCC | |
| TCATGTGTAGTGTAATAAAGGTGTYTGCTGTTCTATYTTAAAAAAAAAAAAAAAAAAAAAAA | |
| Clone 0125-Contig | |
| CTGGAAAGCCHGGATGAGGCTCTTCATCGCTCTTCCTGTCCTGATTGTGGTCGTAGCCATGACCTTGGAAGGCCCAGCCC | |
| CCGCCCAGGCGGCCCCGGATTTGTCCGGAACATTGGAGAGCATACCGGATAAACTGAAGGAGTTTGGGAACACTTTGGAA | |
| GACAAGGCCCGGGCAGCCATTGAACATATCAAACAGAAGGAAATTTTGACCAAGACCCGGGCCTGGTTCTCAGAGGCATT | |
| TGGCAAAGTGAAGGAGAAGTTGAAGACCACGTTCTCCTGAGCACCTGGCGGGCCACCTCGAAGCATCAAGGACATCCACG | |
| TAATGTGCCAGGTCCCTCTTCATCACAGACCAATAAAAAACGTGTAGAAGGCAAAA | |
| Clone 0127-Contig | |
| AGGTTTGAACAGCTTTCTGAATCTGCAAAAGAAGAGCTGATAAACTTCAAACGGAAGAGAGTGGCAGCATTTCGAAAGAA | |
| CCTAATCGAAATGTCTGAACTGGAAATAAAGCATGCCAGAAACAACGTCTCCCTGTTGCAGAGCTGCATCGACTTATTCA | |
| AGAACAACTGACCTGTCTAYTTTAAAGGCCCCCAWTGTGAAAGCCAGCATCACTTGCACTTAAATCATTACTGCAAAAGA | |
| AATAGCTTTGACTAGTTTAAAATCATGTGAATAAATATTTTGATTTCTAAAAATCTTAACACTTAACCATGTTGGTTTAA | |
| AAATATTTTCATTGCATGTTACTTGGACATAACTAATCCTTTTCTTATGCATTTAATACCTCAAAGTAGGTAGAATCTAA | |
| CAGATTCTCCTATAGACTGTCAGGAATTACTAAGAAATGGTATGGANATGTGTGCCTTCTGGAAATGAGAAGCAATAGCC | |
| TGGCCCATGTCTGTCTGAACATTTCCTGTGTCTGCTCCTACAGACATGTCAGGTGTGAATTACAGCAAACAGTGACTGCT | |
| TGTGGGCAGGTGGCAGACACTCTTCTTTATTTAGGTANATTTTGCTCCCTCTTGTCAAGATCACTTCANATGCTTGTACG | |
| GTAACAAGCAAGAAGATCTCATATGTCTTTTTACATCCTTTACAGCTCCATTTTTGTAACTGGGATATAAATACTTGAGA | |
| GAAAGGAGAATCTANAGTTNTGACACTCCCTGATGGTGGGGCATGTGTGTTTGTGCTTTGGATGTTTTTATAAAGTATAT | |
| AATAAAGATAATCCATGTT | |
| Clone 0128-T7 Sequence | |
| GGCGTGCTGACGTCAGGCGCGTGCCCCTGTCCGGCAGCCGAGGAGGCCCCGCGCAGTGCTGCCAACGCCCCGGCGGAGAA | |
| GCTGAGGACAAGATCTAATTTGAAATATTAAAAGTGGATACAAAACTGTTTCCGAAATGCAGACAATTAAGTGTGTTGTT | |
| GTTGGTGATGGTGCTGTTGGTAAAACATGTCTCCTGATATCCTACACAACAAACAAATTCCCATCGGAATATGTACCAAC | |
| TGTTTTTGACAACTATGCAGTCACAGTTATGATTGGTGGAGAGCCATACACTCTTGGACTTTTTGATACTGCAGGGCAAG | |
| AGGATTATGACAGACTACGACCGCTAAGTTATCCACAGACAGATGTTTTTCTAGTATGTTTCTCAGTGGTCTCTCCATCC | |
| TCATTTGAAAATGTGAAAGAAAAGTGGGTGCCTGAGATAACTCACCACTGTCCAAAGACTCCTTTCTTGCTTGTTGGGAC | |
| CCAAATTGATCTCAGAGATGACCCCTCTACTATTGAGAAACTTGCCAAGACAAACAGAAGC | |
| Clone 0131-T7 Sequence | |
| AGAGGGAGGCTCTCAGCGATACGTGCCCGGCCCAGCTTTTTTTTTCTTCAAAATGTCTACTGTCCACGAAATCCTGTGCA | |
| AGCTCAGCCTGGAGGGTGATCATTCTACACCCCCAAGTGCCTACGGGTCAGTCAAACCCTACACCAACTTCGATGCTGAG | |
| AGGGATGCTCTGAACATTGAGACAGCAGTCAAGACCAAAGGAGTGGATGAGGTCACCATTGTCAACATCCTGACAAACCG | |
| CAGCAATGTGCAGAGCCAGGACATTGCCTTCGCCTATCAGAGAAGGACCAAAAAGGAGCTCCCGTCAGCGCTGAAGTCAG | |
| CCTTATCTGGCCACCTGGAGACGGTGATTTTGGGCCTATTGAAGACACCTGCCCAGTATGATGCTTCGGAACTAAAAGCT | |
| TCCATGAAGGGCCTGGGGACTGACGAGGACTCCCTCATTGAGATCATCTGCTCACGAACCAACCAGGAGCTGCAAGAGAT | |
| CAACAGAGTGTACAAGGAAATGTACAAGACTGATCTGCAGAAGCACATCATCTCTGACACATCTGGAGACTTCCGAAAGC | |
| TGATGGTCGCCCT | |
| Clone 0133-contig | |
| CTGGAAAGGCTCCTCGAGGGTCGGTCCCGNCACGTCTNTTCCCGGTCTCTCTGGCTCTGGAGTGCTTTCCCCCCGCGGAG | |
| GGTGGGAGCGCGGGGACAGACGGGCGAGATGAGCACCATGTTTGCAGACACGCTGCTCATCGNCTTTATCTCCGTGTGCA | |
| CCGCGCTGCTCGCCGAGGGCATAACCTGGGTCCTGGTTTACAGGACAGACAAATACAAGAGATTGAAGGCAGAAGTGGAA | |
| AAACARAGTRAAAAATTANAGAAGAAGAAGGAAACTATAACAGAGTCAGCTGGTCGACAACAGAAGAAGAAGATAGAGAG | |
| ACAAGAAGAGAAATTAAAGAACAACAATAGAGACCTGTCAATGGTACGGATGAAATCCATGTTCGCGATTGGCTTTTGWT | |
| TTACTGCCTTAATGGGAATGTTTAATTCCATATTTGATGGTAGAGTGGTGGCAAAGCTCCCCTTCACTCCTCTGTCTTAC | |
| ATCCAAGGACTATCTCATCGGAACCTGCTGGGAGATGACACCACGGACTGCTCCTTCATCTTCCTGTATATCCTCTGTAC | |
| CATGTCAATTCGACAAAACATCCAGAAGATTCTTGGCCTCGCCCCTTCACGAGCTGCCACCAAGCAGGCTGGTGGATTTC | |
| TTTGGCCCACCACCTCCGTCTGGGAAGTTTTCCTGAAGGAAAGCAGAATTCTGAATTTCCTGTCATACTTTTTAGACATT | |
| CACATCAGACTTACAGAGCACCTGGCCACATTCTAGGTTTGTGTTATTTCTCAGCTATGGATATGATCAAATGAGANCAC | |
| TGCTTAACTAATGGGATAGTGCTATGGTCACGGATGGCTTCTTTAGTAATAAGTGCACTCACTGGTTACATTTGGATGGC | |
| TTATGTAACCACAAACATCATCTTTCTTGTCTCACTTGAGTATTTCAGTGTATGTGCCATATACACAGAAGCAGACTGTC | |
| CATGAACATTAGGAGAAAAATAGTTTATGTAACAACTTCACTGTAATTAGTTGTTGTGTTTTTTAAAAAGCCAAATATGT | |
| CACTTTGTGTTCGACAATGAGCCAAATTTTTAGATGTTTCAGTTAGAGAAATGTTTGATTTTTTTGCATATTAAAAAGAG | |
| TATTTTCTGTAATCTGAACATAAGGATAAGCATTGGCCCAAAAAGCTAAAATAAAGTTTTGTCATGTTCTTTT | |
| Clone 0134-Contig | |
| ANCCTCTTCTTGGGNCTAACCTAAAAAAAACCCCCCCCTGGTTTACCTGGGCTTTTTCGGAAATTTNATTNCGGNCCCCC | |
| CCTNTTAAGGGAGNCCCCNAAGCTTTGGGTNCCCAAAGCTCGGGATTCCCCTTATTTACCGGCCCGCCCCATTGTGCTGG | |
| AAAAGGTGGAASCCSCCGCCACGATGCAGATTCTTTGTGAAGACCCCTTMCGGGGAAAACCCWTCACGCTCGAGGTTGAA | |
| CCCTCGCACACTATAGAAAATGTAAAGGCCAAGATCCAGGATAAGGAAGGAATTCCTCCTGATCAGCARAGGCTGATCTT | |
| TGCTGGTAAGCAGCTGGAAGATGGCCGGACTTTGTCTGACTACAACATTCAAAAGGAGTCCACCCTTCATCTGGTGTTGA | |
| GACTTCGGGGTGGTGCTAAGAAAAGGAAGAAGAAGTCTTACACCACTCCCAAGAAGAMCAAGCATAAGAGGAAGAAGGTT | |
| AAGTTGCCTGTGCTGAAATACTATAAGGTGGATGAAAATCGCAAAATTAGCCGACTTCGTCGAGAGTGTCCTTCTGATGA | |
| ATGTGGTGCTGGAGTTTTCATGGGAAGCCACTTTGACAGGCATTACTGTGGCAAGTGTTGTCTGACTTACTGCTTTCAAC | |
| AAACCAGAAGACAAGTAGTTGTGTATGCGTTAATAAAAAGAAGGAACTCGTAAAAAAAAAAAAAAAA | |
| Clone 0136-BGH Sequence | |
| TTTTTTTTTTTTTTTTTTTTTTTTTTTGAAAATTAAATCCAGATATTTAATAGACGGKGCAAACCATAATTTCATCTGAA | |
| TATAAATGTATGCACACGTTTCCAATGAGCTATGTATCAATTATCACAACAGCAATGAGGACCTTAGAGCTACGTTAGAA | |
| AACTGGATAGCTGGAATTAACTCTAACAATCTTATTCGCTTGCAAGAGAACACATCCATTTCAGATCCGTGTCACAAACT | |
| CCAAAAGACCAGGGGGGACAGGAGTGACAAGGGCCAGGATGGTAGACATGGGGCCAGTACACAGGTGACAGACAGAAACA | |
| CAGCTCTTGAGGGAACCAAAACTAGATGTGTTATTACAGCTAAGAACAAGGAAAAGCCTATCTATGGCTTTTAAAAAACG | |
| ACTTTTCTGCCAGTACCGTACACATCTTACAGAGTGAGACACGTCAGCTGCAGGGCTACATTTCCAGTTCGCTTCAACTG | |
| CAAACGGCCGACCCTCCACAGAGGACGCAGCAGTGGGCTTAGCGCAGCCCAGCAACTCAGTGTTGGATTTGCAAATACAG | |
| TTTATTTTACAGGAAATTCAACATTATAAAMNGGGGAACT | |
| Clone 0137-T7 Sequence | |
| AGCAAGGACCCTGAAAATAAACAGCCGCTGCTTTGCGAGTCGCCTTCTTCGTTCTTCGTCCGAGTCTCCTCCGCTGTGGG | |
| CAGCTCAGACGCCGAAGCTCTAACTGCAGCCTATGAGCAGCAACGAATGCTTCAAGTGTGGACGATCTGGCCACTGGGCC | |
| AGGGAGTGCCCTACTGGTGGAGGTCGGCGTCGTGGAATGAGAAGCCGCGGCAGACGGTTCCACTTTCTTTCCTCGTCTCT | |
| CCCTGACATCTGCTACCGCTGTGGTGAGTCTGGTCATCTTGCCAAGGATTGTGATCTGCAGGAGGATGCCTGCTATAACT | |
| GCGGTAGAGGTGGCCACATTGCCAAGGACTGCAAGGAGCCCAAGAGAGAGCGAGAGCAATGCTGCTACAATTGTGGCAAG | |
| CCAGGCCATCTGGCTCGTGACTGTGACCACGCGGATGAGCAGAAGTGCTATTCTTGTGGTGAATTTGGACATATTCAAAA | |
| AGACTGCACCAAGGTGAAGTGCTATACGTGTGGTGAAACTGGTCATGTMGCCATCAATTGCAGCAAGACAAGTGAAGTCA | |
| ACTGTTACCGCTG | |
| Clone 0137-BGH Sequence | |
| TTTTTTTTTTTTTTTTTTCAGTCCTCCACATTCCCCTCTTTAATATGGWATTTTGTTCTATATACATTGATGAAAATTTA | |
| AAACAAACAAAAGGAACTTTAGTCAAAACTCCCCTTCCTCCTCCAGCTTTATTGGAATAGTTTAAAATTACATTTTCTAT | |
| TTTCTAGGACTTCAGTTGCTTTTTCCATCTGACTTTTAAAAACTGATTACAGCAAATGAAACACAGTTGTCTAAGTGATA | |
| TAGTATACAAAAATAACATCTTGATTTCTGTGAAAATGCATTTCTCTGCAATTCCTGAATAGCTCCAAATTATGCTAACT | |
| CTGAGCATCGGATGTTTACTCTGGGTTTTAGATTTAGTCTTTGAAAAAATGTGTTCTACACCTTTGCCATCACCATCTGT | |
| ACGTCCAGCATCGTCACTGGGATGAAGCTGCTTCTGTGTTAGGCTTTGAGCCAGATTCCCTGGCATAGCCTTCAACACAT | |
| TTGTCTTTTCAGTTTACTCCCAACTCAACTGTATATTCTGAGGGCCTGATGCAGATGTTACTCGATGTTATTTAATAGCT | |
| ACTGAGGGTCAGACAATCCAAGGCCGNCATTATGCTAANAATTAAAGGNTATTTATGGAAGCTTAAATGCTTNCAGAATT | |
| GGNTTTACCTTCTACATGGGGAACACACT | |
| Clone 0136-BGH Sequence | |
| TTTTTTTTTTTTTTTTTCGCATATAAATGATTCTTTATGCCCTTCCCCCATGCAGDGGGGGGCGGAGCAGGGGGAAGTCC | |
| CCCGCCTCATGCAGGGAACGGGAGGTCAATGTTTAGTAGTCTTATTGCCAGATGCCCTGTGGCCATGGGGTGGGGAGTGG | |
| GGGGTGTCWACAGCCCCTTGTTTTCTGGCATTGGTCTCCCTTTACCAAGTCACCATGGTGGAGCCAGGGGACTGGAGGCT | |
| TAAAGCTGAGGTAAAGATGGTTCTCTCCAGGAAGAAAAGTGGTTCTCCCCGCTTGCCTGCTGGCCATCCTCAAGGTGAGT | |
| ATGAGGCGGNGTCTGTGGGAANTGGAACGTCACCCAGGGGTCTCTACCCTGTGGNCAGTANGCCGGACAATGTCTC | |
| Clone 0139-T7 Sequence | |
| AGGGACCCCGCATCTTATTAKCAACCAGGGAGATTTCTCCATTTTCCTCTTGTACTACAGTGCGGCCTACAAATCTGGGA | |
| TTTTTTTTTATTACTTCTTTTTTTAAAAAAACTACACTWGGGCTCCTTTTTTGTGCTCGACTTTTCCACCTTTTTCCCTC | |
| CTTCCTGCGCTGCTGCTTTTTTGATCTCTTCGACTAAAAATTTTTTATCCGGAGTATTTAATCGGGTCTCTTCTGTCCTC | |
| CTCGCCACCCCCACCCCCTCCCTCCGGTGTGTGTGCCGCCGCCGCTGNATGCTGCTGCTGCTGCTCGCCCCGTCGTTACA | |
| CCAACCGAAGGCTCTTTGTTTCCTCTCTTGGATCTGCCGAGTTTCTTTGTTGAAGAAGCCAGCATGGGTGCCCAGNTCTC | |
| NAAGACCGCAGCGAAGGGAGAAGCCACCGTCGAGAGGCCCCG | |
| Clone 0139-BGH Sequence | |
| TTTTTTTTTTTTTTTAAATCTGGGTATTGGTGTTTTTATTTTTTTACTCTTTCCTTCTTATTCAAAACGTGTAGTGTTGT | |
| AAACCTGCCTCACAAAATACATCGTAATAATTTTTCTTTAAAAAAAAAAAGAAAGAAAGAAAGAAAGAAAGAAAGAAAGA | |
| AAGAAAGAAAGGACAAGAACCCTAAGCCTTTACACCCACCACCATTTTAGTGGCATATTATCTTGGCAATGTTATGCACT | |
| GCTTCAATTTCCCATAGTGGCCCCTATCACTTCATTTGTTATTCCTTTTGACCCACCCATCTCCTTCATATATGGGTATG | |
| TCTATAGATCTGACAAAGAAAGTTTACACTTTTTTAATAAAGATGCAAAGTATGCAAAAAACATTAATACTGATGCCAAA | |
| AAAAAAAAAAAAAAAGTAAAAGTAAAGAAAGAAGGCAGAGGAAGCTGTCTAACACGTCCTCGGCCTGTCGGAATGGTGGA | |
| AACAACTATCTGAGATGGGATCTGTGGGGAAGGGGGCTTAAAAAAAACANAAAACAAAAAATTTGCTGNTTAANAAAAAT | |
| GTGAAATTTAAAANANAATNTTTAAAATNAATCCCTGGTTGTAGACANGTTNCCAAAAACCAGTACCTGGCACCACTCCA | |
| AC | |
| Clone 0140-Contig | |
| CTGGAAAGCTCGGTTCGGTGTCTGGGGTATGAAGGTCCTGCAAACCCGAGGCTTCGTCTCGGACTCGTCGGATAGCATGG | |
| ATACGGGCGCTGGCTCCATCCGAGAAGCTGGTGGAGCCTTCGGAAAACGAGAAAAGGCTGAAGAGGATCGGTACTTCCGA | |
| GAGAAGACTAAAGAACAGCTGGCTGCCCTGAGGAAACACCATGAAGATGAGATTGACCACCATTCGAAGGAGATAGAGCG | |
| TCTGCAGAAGCAAATTGAACGCCATAAGAAGAAGATCCAACAACTAAAGAATAATCATTGAATGCGCGCAGTCGGTCCCT | |
| CACAGAGTGGCCCGTATCACTCCCCACGTCTGTAGACACATGGCTTTGAATGATTACTATTTGGTCTGTGTGCTACTAAC | |
| AGATAATAACGATCACCAGGAAAAAAAAAAAAAAAAA | |
| Clone 0141-Contig | |
| AGGGAGGTAGAGGTGMCCGATCCTGGGGGTCGGTGTTAGGCCGTGGGAGGTTTTTCGCGAGACTGAGCCACGCGTCTATC | |
| TTCTGTCCCAGGGCCGCCGTCACCATGGGCCGCGAGTTTGGGAACCTGGCGCGGATACGGCACGTGATCTCCTACAGCTT | |
| GTCGCCCTTTGAGCAGCGCGCCTTCCCAAGCTATTTCAGCAAAGGCATCCCCAACGTGCTGCGCCGCACTCGCGAGCGCA | |
| TCCTGCGCGTGGCGCCGCCGTTTGTAGTGGTCTACCTGATCTACACATGGGGCAACCAGGAGTTTGAGCAGTCGAAAAGG | |
| AAGAATCCAGCCATGTATGAAAATGACAAGTAGACGGCCTGCACCTGGGTGACAGTCCCCTGCCTCTGAAAGACCCTTCT | |
| CTGGGAGAGGAATCCMCMCTGTAGTCTTGAAGMCAATAAACTACTTATGGMCTTCCAAAAAAAAAAAAAAAAAAAAAA | |
| Clone 0142-T7 Sequence | |
| CGTTGTCTTGCTCCGGCTGCGCGCATTGTCCTCAGGGTCCTCCGACAGGGCTGCTGCGGGGCCCGGGACCCGCGCCCTAG | |
| GGACGCGCCCCCGCTGCCGGTCGGCCTGGCGCGGGGCTCTGCTAGTCTGTTGGCGAGCCCGTGCTACCGGGCTAGTCTCG | |
| CCGGGGTTTTTCCTGCGAAGTTGAGGAAGGGGAGAAGTCCCACCCGTCCGCCCAGCCCAGCCTTNCCCGGCGCGCAGCCT | |
| CGACGGGGCCGTGGCAGGCGCGACGAGGGCCGCCGACGGAGCCCTGAGAGAAGTACAAAGTGGTTNGTAACTGGGCTCGN | |
| Clone 0142-BGH Sequence | |
| TTTTTTTTTTTTTTTCAGGTTTGAAACTTTTGCTTGCCCGTGTCAAGCAGCCAATACAGAGCCTTAATGGCCTATTTAAA | |
| CGGGGAAATATGCATGAACCAAAATCAACGAGGAACGTACACGCTGTATAGAGCCATTTTTCCACCGGGGTCTCAGATGC | |
| TCTGCAACGTACACCCTCTCAGACCCTGGCTGAAGGCTGGGGATGCTGGCTACACAAGTCAGCCAGGTTCCTCGGACATC | |
| CCAGCCAAGACAACAAAGTCGTCTCCAATCTCAAAGCAAGAAGACAATCACATTAAGAGAATCGGGTGCGTTTGGCTTTG | |
| TGCACAGAGCGTGGCCAGCGCCCGGCGGCAGACGGCGCCTCACAGGATCACGCAGGCCGAGCAGCAGCCTCGTCGCCGTT | |
| GGGCTGTGCCGCGTAGTTCATCTGCCTCACGATCTCTGCGAATAGCTCATCCACTGAGGCTTTGTTTTTGGCCCGATGTC | |
| TCCATGAAGGGGCAGCTCCACTCCTCGGCCAGGGCCTTACCCTCGCCATAGGAGACCTCACGTTCACCCTCCAAGTCCAC | |
| CTTGTTGCCTACCAGGATCATGGGGTACGCGCTCGTACCGCTTCACGCGGATGATCTGGGCCCGCATGGGCTTGATGN | |
| Clone 0143-T7 Sequence | |
| TGGNAAGGGGGGACTTTTGGTATAGCATTGGAAATGTAAATGAGCTAAATACCTAATAAAAAAATGGAAAAAAAAAAAAA | |
| AAAAAAAAAAAGAAMTAGAAAAAATGTTTACTGAGGTCTTTATGTCTGTTGTTTTGTTTTGTTTTAGAAGTTTCTGAACG | |
| TTGTTTTAGGACTAAACTGGGAAGCCATGTGTCTTCGTTTTGGATTTTATAGATAACCTGAAGCATTATTGAGATTCCTT | |
| CAAAGTCAGTAACAACTTATTGGGTGTTTCAAGCCAGTTGTGAGTCTCTGAGTATTTGCCCAGNTTCACATTTATTGGTA | |
| AAGGTCCCTTACAAG | |
| Clone 0143-BGH Sequence | |
| TTTTTTTTTTTTTTTGACTCTTGCTACTTGTGAGGCTCAGCAAAAAGAATTCTTTACAAGGTGTAACCCAGATTATGAGA | |
| ACTATAGATGGAATCAGTCTGTACCACTCTGTGCCATTGTCCTCTGAGTAGGTTCTCTGTGACTGTAGATTATTTTTCCA | |
| GTCTATAAAAGGTCATGGGTGCTATTGGAACTCTAGTCAGGCATCAACTTTAGGCCATCATTTCTCCATCAAAGGAAAGA | |
| GAGCTTCTACATTTCGCAGACATGAGCATCCTAGATTACCCATAGTGCTCAAGTGTCATTTGAGTAATTTATTGAGATAG | |
| CAAAGGCCTTTTGCATCTGATCATGCTGGGAAGAATGGAGTAAGTTTGGCATCCTTACACCTACCGAGTTGGCGACAGAC | |
| ATATTCCTCAGGAGGTTGGAATGAAATTTTTGAATGTTCAGAGGAAACCTATATACATTTAAGGTGAAATCTAAAGAATA | |
| AGAGAGAAAAACAAAACAAAACAACAAAAGAAACTCTTACCATTTGAGCACTAAGAATAGTCAAAGCAGAGGAAACAGGC | |
| TAGTGCAAAGGTCCATCTAGTACAAGAGAGNTTATGGCTTGCTCCAGGAACGGACAGAAGGCTAGCGNGGCTGGCG | |
| Clone 0144-Contig | |
| AGGGACAGCTCCCCTTTCTGGAAGGTGAAATTTTTGATTCTGTGAAGCCGGGACTTTCTGCTTTTGTGGATCAGCCCAAA | |
| CAGGGTGCTGAGACTGTCCAGGAGCTCTTGGAGGTGGCCAAAGACTCGATCCCCAGAAGCCACTGGGAAAGGACCCCGGT | |
| GGTTCTGAAAGCAACGGCCGGACTCCGTTTGCTGCCTGAGCAGAAAGCCCAGGCTCTGCTCTTGGAGGTAGAGGAGATCT | |
| TCAAGAATTCACCTTTCCTGGTCCCAGATGGCAGCGTTAGCATCATGGATGGGTCCTATGAAGGCATACTAGCCTGGGTT | |
| ACCGTGAACTTTCTAACAGGTCAGCTGCATGGTCGTGGCCAGGAGACTGTGGGGACCCTTGACCTGGGGGGTGCCTCCAC | |
| CCAAATCACGTTTCTACCCCAGTTTGAGAAAACCCTGGAACAAACACCTAGGGGCTACCCTCACTTCCTTTGAGATGTTT | |
| AACAGCACTTTTAAGCTCTATACACATAGTTACTTGGGATTTGGACTGAAAGCTGCAAGACTGGCAACTCTGGGAGCCCT | |
| GGAAGCAAAAGGGACTGATGGACATACGTTTCGAAGTGCCTGTTTACCAAGATGGTTGGAAGCAGAGTGGATCTTTGGGG | |
| GTGTGAAATACCAGTATGGTGGTAACCAAGAAGGGGAGATGGGCTTTGAACCCTGCTATGCGGAAGTGCTGAGGGTAGTA | |
| CAGGGGAAACTTCACCAGCCAGAAGAAGTCCGAGGAAGCGCCTTCTACGCTTTCTCTTACTACTACGATCGACCCGCTGA | |
| CACACACTTCATCGATTATAAAAA | |
| Clone 0145-Contig | |
| AAAAGGGCAGCCATGGCGCCCAGCCGGAATGGCATGATACTGAAGCCCCACTTCCACAAGGATTGGCAGCAGCGAGTGGA | |
| CACTTGGTTCAACCAGCCGGCGCGCAAGATCCGCAGGCGCAAGGCCCGGCAGGCGAAAGCGCGTCGCATCGCCCCTCGCC | |
| CCGCGTCCGGCCCCATCAGGCCCATCGTGAGGTGCCCTACAGTGAGATACCACACCAAGGTCCGGGCTGGCAGGGGCTTC | |
| AGCCTGGAGGAGCTCAGGGTGGCTGGCATCCACAAGAAAGTGGCTCGCACCATCGGCATCTCTGTGGACCCGAGGAGGCG | |
| AAACAAGTCCACCGAGTCACTGCAGGCCAACGTCCAGCGCCTGAAGGAGTACCGCTCCAAGCTCATCCTGTTCCCCAGGA | |
| AGCCTTCTGCTCCGAAGAAGGGAGACAGTTCTGCTGAAGAACTTAAGCTGGCCACCCAGCTGACAGGACCTGTGATGCCC | |
| ATCCGGAATGTGTACAAAAAGGAAAAGGCCAGAGTTATCACAGAAGAAGAGAAGAACTTTAAGGCTTTTGCCAGTCTCCG | |
| AATGGCCCGTGCCAATGCCCGACTCTTTGGCATCCGAGCAAAAAGGGCGAAGGAAGCTGCAGAGCAAGATGTTGAAAAGA | |
| AAAAATAATTCTGTGTTGGAGAGCTGCAATAAATTTTCCATAAAGCAAAAAAAAAAAAAAAAAAA | |
| Clone 0146-Contig | |
| AGGGCGAAGAGTGAGTGCCAAGGTTCATATGGGAAGGACTTTGGGGTGAGCATCTTCTCTATTTCCAGCTGGCTTTTCTG | |
| ATTTTCAGAAAGAAGACTCATCAAAGATGTCCAGCAAGACGGCAAGCACCAACAGCATAGCCCAAGCCAGGAGAACTGTG | |
| CAGCAGCTGAGATTGGAAGCCTCCATCGAAAGAATAAAGGTCTCAAAAGCATCAGCAGACCTGATGTCATACTGTGAGGA | |
| GCATGCCCGGAGCGACCCTCTGCTGATGGGCATACCGACCTCAGAAAACCCGTTCAAGGATAAGAAGACCTGCATCATCT | |
| TATAGTGGACCAGGAAGCGCCCCTTGCCTCTTAACGCAAACCACAGCAGCAACCTGAAGGGATTCCTTCAGCTTACCTGG | |
| TAACCACAGCTAGTAACTAAAACACCCTTCTCTCGGAATAATAGACCCTGAAGTCTCTCTTTTTCAAGTTGTCCTTTCTT | |
| CACCCTTTACTGATTTAATACAGAATAGCAATCTTATTTTCTATTTGATAACTATGGTATCATATTGGGTTACTGTATAA | |
| GGAAAATGGCAGGGGAGTTGTGGGAAGCTTGTCTTTACAAAATATAATTGATTAAGATATGTCAAGACCTACATTGTCTA | |
| AGCACCGGCAAATTAAAATGTCGAGAATCACTTCAGTCAAAAACCTTTATATTCTGTTCTTAATAATGTTTGTGCCAACC | |
| TATATCCCATGTAAGGGATCTGGGGAGGAGGCATGTGTCTACAACCATACCTTTTTGCACTATGGGCACTAACCACCCTG | |
| AAACTTCCTGCGGTAGCTCCCTCCCTTCAGAGTTACATCATTATCCTGACTCTGTGTAGGTAAATTTTCGTGAAATTTTT | |
| GTACAAAAAAAAAAAAAAAA | |

1. An apoptosis-associated nucleic acid comprising:
(a) the nucleic acids of clones 1-124, shown in table 1 or in SEQ ID NO: 1-225, or the nucleic acids of clones 1-124, 125, 127, 133, 134, 140, 141, 144, 145, 146, shown in table 2, nucleic acids complementary thereto or fragments thereof,
(b) nucleic acids corresponding to the sequences as claimed in (a) within the framework of the degeneracy of the genetic code and
(c) nucleic acids hybridizing with the sequences as claimed in (a) or/and (b) under stringent conditions.

2. The nucleic acid as claimed in claim 1, characterized in that said nucleic acid induces apoptosis in a cell after expression.

3. The nucleic acid as claimed in claim 1 or 2, characterized in that said nucleic acid codes for an apoptosis-associated polypeptide.

4. The nucleic acid as claimed in any of claims 1 to 3, characterized in that said nucleic acid originates from a eukaryotic organism.

5. The nucleic acid as claimed in claim 4, characterized in that said nucleic acid originates from a mammal.

6. The nucleic acid as claimed in claim 5, characterized in that said nucleic acid is of human origin.

7. The nucleic acid as claimed in claim 1, characterized in that the part fragments have a length of at least 15 nucleotides.

8. The nucleic acid as claimed in any of claims 1 to 7, characterized in that said nucleic acid is operatively linked to an expression control sequence.

9. The nucleic acid as claimed in claim 8, characterized in that the expression control sequence is a heterologous expression control sequence.

10. A recombinant vector, characterized in that said recombinant vector contains a nucleic acid as claimed in any of claims 1 to 9.

11. A recombinant cell, characterized in that said recombinant cell is transformed or transfected with a nucleic acid as claimed in any of claims 1 to 9 or a vector as claimed in claim 8.

12. A polypeptide, characterized in that said polypeptide is encoded by a nucleic acid as claimed in claim 1.

13. A pharmaceutical composition comprising a nucleic acid as claimed in any of claims 1 to 9, a vector as claimed in claim 10 or a polypeptide as claimed in claim 12, where appropriate together with pharmaceutically conventional carriers and excipients.

14. The use of a nucleic acid as claimed in any of claims 1 to 9, of a vector as claimed in claim 10, of a cell as claimed in claim 11 or of a polypeptide as claimed in claim 12 for producing a diagnostic or therapeutic agent.

15. The use as claimed in claim 14 for the diagnosis, therapy or prevention of apoptosis-associated disorders.

16. The use of a nucleic acid as claimed in any of claims 1 to 9, of a vector as claimed in claim 10, of a cell as claimed in claim 11 or of a polypeptide as claimed in claim 12 for identifying active substances for the therapy or prevention of apoptosis-associated disorders.

17. The use as claimed in claim 16, characterized in that identification is carried out in a high-throughput method.

18. The use as claimed in claim 16 or 17, characterized in that the active substances activate or inhibit signal pathways induced by expression of the nucleic acid.

19. A transgenic nonhuman animal,
(i) which constitutively or inducibly overexpresses the gene of a nucleic acid as claimed in any of claims 1 to 9 or the ANT-1 gene,
(ii) which contains the endogenous gene of a nucleic acid as claimed in any of claims 1 to 9 or the ANT-1 gene in an inactivated form,
(iii) in which the endogenous gene of a nucleic acid as claimed in any of claims 1 to 9 or the ANT-1 gene has been replaced completely or partially by a mutated gene of a nucleic acid as claimed in any of claims 1 to 9 or by a mutated ANT-1 gene,
(iv) which over- or underexpresses conditionally and tissue-specifically the gene of a nucleic acid as claimed in any of claims 1 to 9 or the ANT-1 gene, or
(v) which has a conditional and tissue-specific knockout of the gene of a nucleic acid as claimed in any of claims 1 to 9 or of the ANT-1 gene.

20. The transgenic animal as claimed in claim 19, characterized in that the endogenous promoter of the gene of a nucleic acid as claimed in any of claims 1 to 9 or of the ANT-1 gene has a genetic modification which results in a modified expression of said gene.

21. The transgenic animal as claimed in claim 19 or 20, characterized in that it is a rodent, in particular a mouse.

22. The use of a transgenic animal as claimed in any of claims 19 to 21 for the genetic and/or pharmacological investigation of apoptotic processes, in particular of disorders which are associated with increased or reduced apoptosis.

23. A cell culture
(i) which overexpresses constitutively or inducibly the gene of a nucleic acid as claimed in any of claims 1 to 9 or the ANT-1 gene,
(ii) which contains the endogenous gene of a nucleic acid as claimed in any of claims 1 to 9 or the ANT-1 gene in an inactivated form,
(iii) in which the endogenous gene of a nucleic acid as claimed in any of claims 1 to 9 or the ANT-1 gene has been replaced completely or partially by a mutated gene of a nucleic acid as claimed in any of claims 1 to 9 or by a mutated ANT-1 gene,
(iv) which over- or underexpresses conditionally and tissue-specifically the gene of a nucleic acid as claimed in any of claims 1 to 9 or the ANT-1 gene, or
(v) which has a conditional and tissue-specific knockout of the gene of a nucleic acid as claimed in any of claims 1 to 9 or of the ANT-1 gene.

24. The cell culture as claimed in claim 23, characterized in that it consists of human cells.

25. The use of a cell culture as claimed in claim 23 or 24 for the genetic and/or pharmacological investigation of apoptotic processes, in particular of disorders which are associated with increased or reduced apoptosis.


SEQUENCE LISTING (355 sequences)

SEQ ID NO: 1 - DNA, 845 bp, Mus musculus
  misc_feature (612)..(845): n = any nucleotide
agctcatgttggacagtcgtgtgaggagctatggagcacagcagtaatcgcccagaggacttcccgcttaacgtgttctctgtcactccgtacacacccagtaccgccgacatccaggtgtccgacgacgacaaggcaggggccactttgcttttctcaggcatctttctaggactggtggggatcactttcactgtcatgggctggatcaaataccaaggtgtctcccactttgaatggacccagctcctcggacccatccttctgtcggtcggagtgacattcatcctgatcgctgtgtgcaaattcaaaatgctatcctgccagttgtgctcagataacgaggagagggtcccggactcggaccagacttccggaggacagtcgttcgttttcactggcatcaatcagcccatcaccttccacggggccaccgtggtgcagtatatccctcctccttacggttctcaggagcccctgggaatgaacgccacctacctgcaacccatgatgaatccttgcggtctcatacctcctagtggagcagcggctgccgcaccaagtccccctcagtactacaccatctaccctcaagacaatgctgcgttcgtngagagtgagggcttctctcctttcgtgggcactggatatgacaggcccgactctgatgctgaccagctagaagggacggagttggaagaggaggactgcgtatgttctcttctccaccgnatgaggagaatacgctctacctcgctngagactgcaanctaagggacggcatttaagcccctgngatgngaacttgngngnnaaccntgggtctttanaagtaggngnaaacn

SEQ ID NO: 2 - DNA, 400 bp, Mus musculus
  misc_feature (300)..(395): n = any nucleotide
atagacatgtctagttttttattactagttatcatccaagtgaaatgtccctgaggcataatatgaatcacaataataaaccaagattgttttgctgatatccttgaaaaacctggactcttctgacagagagtaaaagcaatcccacatataagcacagtaccaaaaccttcaagacctgacaattcctgtatctctcttgggggcagccacctttaccttgagtggcctttgatctttgtaaaactgttttctttctaattctttggacacctcctaaagtctgcagctttgggctgnggttggtgacaccgttcagacttaggaagctgaaggctaccagcttttctttgttcaaatgaaggtgagagctacgcccggnggcgggntaganngagac

SEQ ID NO: 3 - DNA, 460 bp, Mus musculus
  misc_feature (10)..(436): n = any nucleotide
ttcccacgcngntgngggcttctctcctttcgtgggcactggatatgacaggcccgactctgatgctgaccagctagaagggacggagttggaagaggaggactgcgtatgtttctctcctccaccgtatgaggagatatacgctctacctcgctagagactgcaatgctaaggggacggacatttaagccctgngatgtgatacttggagagtttatcgctgagttcttcagaagttaggtgtcaaagcagctnaggagatcttacagatgtcattnaaggngggaaagaagtgcccngagactgctaaattaagctgccctggttaaattcccctctgctctggttttgaattctctcagctaagaaaccctctgcagctggagagtcgctctgagatagagagatttnggagcccacgcagngcctngggctngatctctagagccagaagaaaaca

SEQ ID NO: 4 - DNA, 713 bp, Mus musculus
  misc_feature (10)..(688): n = any nucleotide
tcccagcctnttctntgntcaaatgaaggtnagagctacgcccgggggcggagggngggggagacgggatgactgatcatctccatgattcatgactacaaagcgacacggcagnatggctctgnggaacagatcagaggcatacctgtgttccataagcnactcagctgtacctgcgttctgtaagccactcggctgagctagttcgngcctgggaatcacagcctgggggngggcagagggagcaggcgctnaccnatgtacattacgccttgaacttcanccttgcaaaggaaaacnatcangagcaggggtcactntaactcggnggacagcacatggngacattcattatcctcctgntacctcgtccacagtaggatcggccacccaacactattctaacatggggcactgtttcatccnactctacttattcatttatttcttaatggattttatttctttaacaaactcttnaagatccaaaggcttccagngagaccanataanagtcnaagttgtctaanataagtnactctgctgcggaaggttcagngccgtgcagggaaatttctacctgagcctgctctcttccctgcttgcttgccagcctccctttnatcantctnagagctgccatggctgcccggatttaaaactaanacaatctttnaaaactaagatntcctttaaatggatattccgcatgg

SEQ ID NO: 5 - DNA, 2269 bp, Mus musculus
aattcggatccatgcccaaaggtccttctctaactaaccggagcatggtgttccccactctcacactctgtttcctctcatgttggacagtcgtgtgaggagctatggagcacagcagtaatcgcccagaggacttcccgcttaacgtgttctctgtcactccgtacacacccagtaccgccgacatccaggtgtccgacgacgacaaggcaggggccactttgcttttctcaggcatctttctaggactggtggggatcactttcactgtcatgggctggatcaaataccaaggtgtctcccactttgaatggacccagctcctcggacccatccttctgtcggtcggagtgacattcatcctgatcgctgtgtgcaaattcaaaatgctatcctgccagttgtgctcagataacgaggagagggtcccggactcggaccagacttccggaggacagtcgttcgttttcactggcatcaatcagcccatcaccttccacggggccaccgtggtgcagtatatccctcctccttacggttctcaggagcccctgggaatgaacgccacctacctgcaacccatgatgaatccttgcggtctcatacctcctagtggagcagcggctgcgcaccaagtccccctcagtactacaccatctaccctcaagacaatgctgcgttcgtggagagtgagggcttctctcctttcgtgggcactggatatgacaggcccgactctgatgctgaccagctagaagggacggagttggaagaggaggactgcgtatgtttctctcctccaccgtatgaggagatatacgctctacctcgctagagactgcaatgctaaggggacggacatttaagccctgtgatgtgatacttggagagtttatcgctgtgttcttcagaagttaggtgtcaaagcagctcaggagatcttacagatgtcattcaaggtgggaaagaagtgccccgagactgctaaattaagctgccctggttaaattcccctctgctctggttttgaattctctcagctaagaaaccctctgcagctggagagtcgctctgtgatagagtgattttggagcccacgcagtgccttgggtttgatctctagagccagaagaaaacaaaaacaaaaacaaaaacaaaacaagacctctctacataaagtgcaggaggaaaattcacccatttccccatcccccacccgatatccatttgaaggatatcttagttttgaaagattgtcttagttttaaatccggcagccatggcagctctcagactgatgaaagggaggctggcaagcaagcagggaagagagcaggctcaggtagaaatttccctgcacggcgctgaaccttccgcagcagagtgacttatcttagacaacttgggctgttatctggtctccctggaagcctttggatcttgaagagtttgtaaaagaaataaaatccattaagaaataaatgaataagtagagtgggatgaaacagtgccccatgttagaatagtgttgggtggccgatcctactgtggacgaggtaacaggaggataatgaatgtcaccatgtgctgtccaccgagttacagtgacccctgctcctgatggttttcctttgcaaggctgaagttcaaggcgtaatgtacatgggtgagcgcctgctccctctgcccacccccaggctgtgattcccaggcacgaactagctcagccgagtggcttacagaacgcaggtacagctgagtggcttatggaacacaggtatgcctctaatctgttccacagagccatgctgccgtgtcgctttgtagtcatgaatcatggagatgatcagtcatcccgtctcccccaccccccgcccccgggcgtagctctcaccttcatttgaacaaagaaaagctggtagccttcagcttcctaagtctgaacggtgtcaccaaccacagcccaaagctgcagactttaggaggtgtccaaagaattagaaagaaaacagttttacaaagatcaaaggccactcaaggtaaaggtggctgcccccaagagagatacaggaattgtcaggtcttgaaggttttggtactgtgcttatatgtgggattgcttttactctctgtcagaagagtccaggtttttcaaggatatcagcaaaacaatcttggtttattattgtgattcatattatgcctcagggacatttcacttggatgataactagtaataaaaaactagacatgtctaaaaaaaaaaaaa

SEQ ID NO: 6 - DNA, 694 bp, Mus musculus
  misc_feature (602)..(602): n = any nucleotide
agctcggcgccgcctgagcgcccggcccgaccccgccatggggtgctgctatagcagcgaaaacgaggactcggaccaggatcgggaggagaggaagctgctgctggaccccagtagcacccctaccaaagccctcaatggagccgagcccaactaccatagcctaccttcagctcgcacagatgagcaggccctgctttcctccatccttgccaagacagctagcaacatcattgatgtgtctgccgcagactcccagggcatggaacagcatgagtacatggaccgggcaaggcagtacagtacccgcttggctgtgcttagcagcagtctgacccattggaagaagctgccaccgttgccatctctcaccagccagccccaccaagtgctggccagtgagcctatccccttctctgacttgcagcaggtctccaggatagctgcgtatgcctatagtgcactttctcagatccgcgtggatgcgaaagaagagctggttgtacagtttgggatcccatgaagagaggggccctaggacagctcttccctcgtcttcaccccgtctccaccccacctcttctggcccccagcctcactgnggctctctacagtacctaacctgctactaatcacggagaagaatgtggagggaaagaacaaggctggaggccggagcaagtgaggactaagc

SEQ ID NO: 7 - DNA, 625 bp, Mus musculus
  misc_feature (228)..(468): n = any nucleotide
caaatgaatatactttctttatcgaggggtgacaaacaaaaacaaaaagagcaaacatgtaaaaacccagggtgctagaaatacaaactcaattcagactcaagctcgtctagaccctggtcataatccccagtgaggtgcctgtgagcaccaagtcagggaaggggacaggagtgaatcggaggccaagagaaagagggcaggaagggatctcctaggtctcccggngtcacccctacagnggtatctccatcttcccaatgactgaagatctgccaggccctgtcctcttggccccaacctnaccctaaccagagcatgaaggccgatggcaatcggtcctcccttcccttgcttagtcctcacttgctccggcctccagccttgttctttccctccacattcttctccgtgattagtagcaggttaggtactgtagagagccacagtgaggctgggggccagaanaggtggggtggagacggggtgaagacgagggaagagctgtcctagggcccctctcttcatgggatcccaaactgtacaaccagctcttctttcgcatccacgcggatctgagaaagtgcactataggcatacgcagctatcctggagacctgctgca

SEQ ID NO: 8 - DNA, 1047 bp, Mus musculus
  misc_feature (744)..(820): n = any nucleotide
agctcggcgccgcctgagcgcccggcccgaccccgccatggggtgctgctatagcagcgaaaacgaggactcggaccaggatcgggaggagaggaagctgctgctggaccccagtagcacccctaccaaagccctcaatggagccgagcccaactaccatagcctaccttcagctcgcacagatgagcaggccctgctttcctccatccttgccaagacagctagcaacatcattgatgtgtctgccgcagactcccagggcatggaacagcatgagtacatggaccgggcaaggcagtacagtacccgcttggctgtgcttagcagcagtctgacccattggaagaagctgccaccgttgccatctctcaccagccagccccaccaagtgctggccagtgagcctatccccttctctgacttgcagcaggtctccaggatagctgcgtatgcctatagtgcactttctcagatccgcgtggatgcgaaagaagagctggttgtacagtttgggatcccatgaagagaggggccctaggacagctcttccctcgtcttcaccccgtctccaccccacctcttctggcccccagcctcactgtggctctctacagtacctaacctgctactaatcacggagaagaatgtggagggaaagaacaaggctggaggccggagcaagtgaggactaagcaagggaagggaggaccgattgccatcggccttcatgctctggttagggtnaggttggggccaagaggacagggcctggcagatcttcagtcattgggaagatggagataccnctgtaggggtgacnccgggagacctaggagatcccttcctgccctctttctcttggcctccgattcactcctgtccccttccctgacttggtgctcacaggcacctcactggggattatgaccagggtctagacgagcttgagtctgaattgagtttgtatttctagcaccctgggtttttacatgtttgctctttttgtttttgtttgtcacccctcgataaagaaagtatattcatttg

SEQ ID NO: 9 - DNA, 637 bp, Mus musculus
  misc_feature (17)..(625): n = any nucleotide
agtctggcttcacgctncannagtgncgagcgcctcacggaggaagagttgcatatcatcgcgcaggtgccgcctccacccgccgctgccgccgttggctacgctggctgccgtgtggctgtgaccttcttcctctacttcctggctaccaactactactggattctggtggagggactgtacttacacagcctcatcttcatggcctttttctcagagaagaagtatctgtggggcttcaccatctttggctggggtctgccggctgtcttcgtggctgtgtgggtcggtgtcagagcaaccttggccaacactgggtgctgggacctgagctctgggcacaagaagtggatcatccaggtgcccatcctggcatctgttgtgctcaacttcatcctctttatcaacatcatccgggtgcttgccactaagcttngggagacaaatgngggccggngtgacaccaggcantagtaccggaagctgctnacgtcacnttggtgccngcgccactctttttgtcactacacccgtcttnatggccttnccgtacacccgaggtcttnagggacactttgncanatccagatcactatnagantgcttctttaaacttccttttcangggatttttttt

SEQ ID NO: 10 - DNA, 385 bp, Mus musculus
  misc_feature (27)..(381): n = any nucleotide
cccttcttttttttttttttttttctnttttcctttngtnttccnggnccagtcctgaanagacagccagccaaantttggnttntnggnccatntgtccatntgcccatgccagcagtccagcccnttgangcccagtcanatgactgnttcccattnttcctgcaanaanggaggnggccgcgcaaacccanaggcctnntaatccanacccgagcaggagccattaaggaagccgtcgtccttgggaactgncanagnaactggnanggtttcgttntaaanggntggagcncccggnttggcgnggccaggcagntggnagnggccantggnggnggnaggaagnaggcggnagntaaggggaaggntgagtcctgnacgg

SEQ ID NO: 11 - DNA, 746 bp, Mus musculus
  misc_feature (423)..(737): n = any nucleotide
aggcgctgcctaccagagcgcagcatgacggccatcggcgcgcaggcccacaagctgttgggccttaagaggccccaccggtctttctttgagtccttcatccggacactcatcatcgtgtgcactgccctggctgtggtcctttcttcagtctccatctgcgatggccactggctcctagtggaggatcatctctttgggctgtggtacttctgcaccatcggcaaccacagtgaaccacactgtctgagagacctgagccaggcccatatgcccgggctggctgtaggcatgggcctagcacgcagtgtggccgccatggcagtggtggctgccatcttcggcttggagatgctcattgtgtcccaggtgtgtgaagatgtccgctcacggcgcaagtgggccatcggttcctacctcctnctggttgcctttatcctctcctctgggggcctcctcaccttcatcatcctgctcaagaatcagatcaaccttctgggcttcaccctgatgttctggtgtgaattcactgnctncttcctcttcttnctnaatgccgccagcgggctttacatnaaaagcttacttagcccttggaccctcagcagggaccctggcttncanaaagngaggttatgatgggactttttttattntagggaanctnttgnctggngatttgcncccccccccnccccaatgnncctttgacacnttccctctttatgggtaatttnaagtttttt

SEQ ID NO: 12 - DNA, 614 bp, Mus musculus
  misc_feature (174)..(387): n = any nucleotide
actttcaaattgagattttaatagcatgactaacctatccagctcactgtgccgtcgtacagaggcacccttctgcctttgccctggagctcagctgaagagacttccagggcattgctagagctaagtgcctaagaggcagtgtcaaggtcattggttgtcctattcaatctnagccagaggtcctatatnagagaagtcccatcataacctcgctttctgtaagccagggtccctgctggagggtcccagggctgagtgaggctgttgatgtgaaggccgctggcggcattgaggaagaagaggaaggaggcagtgaattnacaccagaacatnagggtgaagcccaggaggttgatctgattcttgagcaggatgatnaangtnaggaggcccccagaggagaggataaaggcaaccaggaggaggtaggaaccgatggcccacttgcgccgtgagcggacatctttacacacctgggacacaatgagcatcttcaagccgaagatggcagccaccactgccatggcggccacactgcgtgctaggcccatgcctacagccagcccgggcatatgggcctggctcaggtctctcagacagtgtggtcactg

SEQ ID NO: 13 - DNA, 640 bp, Mus musculus
  misc_feature (457)..(632): n = any nucleotide
agcagactcaggaagaaaccatggtgctctctggggaagacaaaagcaacatcaaggctgcctgggggaagattggtggccatggtgctgaatatggagctgaagccctggaaaggatgtttgctagcttccccaccaccaagacctacttccctcactttgatgtaagccacggctctgcccaggtcaagggtcacggcaagaaggtcgccgatgctctggccaatgctgcaggccacctcgatgacctgcccggtgccctgtctgctctgagcgacctgcatgcccacaagctgcgtgtggatcccgtcaacttcaagctcctgagccactgcctgctggtgaccttggctagccaccaccctgccgatttcacccccgcggtgcatgcctctctggacaaattccttgcctctgtgagcaccgtgctgacctccaagtaccgtaagctgccttntgcggggcttgccttctggccatgcccttcttctctccttgcacctgtacctctttggttttgaataaagcctgagtaggaagaaaaaaaaaaaaaaanagggcggncgttngagcatgcatctagaggggccctattntatagtgnnacctaaaaatgcttagagntttgntgntcagccct

SEQ ID NO: 14 - DNA, 817 bp, Mus musculus
ggtaccgagctcggatccactagtaacggccgccagtgtgctggaaaggggcttcggacccggaagtggcgccttgggctcccggcggcgccgcggggatggcgggagccggagctggtgcaggagctcggggcggcgcgccggccggagtcgaggcccgcgctcgggacccgccacccgcgcaccgcgcgcaccctcgccatcctcggcccgcggctcagccgtcggcgcgcaggatggacggcggcccgggcgccccgggctccggggacaacgccccgaccaccgaggcgctgttcgtggcgctgggcgcgggcgtgacggctctcagtcacccgctgctctacgtgaagctgctgatccaggtgggtcatgagccgatgccccccacccttgggaccaatgtgctggggaggaaggtcctctacctgccgagcttcttcacctatgccaagtacattgtgcaggtggatgggaagatagggctcttccggggcctgagcccccgccttatgtccaacgccttgtccactgtgacccgcggcagcatgaagaaggttttccctccagatgagatggagcaggtttccaacaaggacgacatgaagacctcactcaagaaagttgtgaaggagacatcgtatgagatgatgatgcagtgtgtatcgcgaatgctggcccatcccttacacgtgatctcgatgcgatgcatggtgcagtttgtgggacgggaggccaagtacagtgtgtgctgagttctattgggagatcttcaaggaagagggctgctgggattcttcgttggcttaatccctca

SEQ ID NO: 15 - DNA, 634 bp, Mus musculus
gacttgaactcaggggaattcggagggagtccagcccgcagcccacagttgttcactgccatgagatctccaacgagcaggaaggggtaggtcagcatgctcactgcaatccccatcacaaacttggtgtagctccggatggccagggcctggctaaagctgtcgtccaccaagtaggcattgatgaagtgggccagcaggttacagccccacaagaaaaccacatcgcccaggaggtgagggattaagccaacgaagaatcccagcagcccctcttccttgaagatcttcccaatagaactcagcacaccactgtacttggcctcccgtcccacaaactgcaccatgcatcgcatcgagatcacgtgtaagggatgggccagcattcgcgatacacactgcatcatcatctcatacgatgtctccttcacaactttcttgagtgaggtcttcatgtcgtccttgtttggaaacctgctccatctcatctggagggaaaaccttcttcatgctgccgcgggtcacagtggacaaggcgttggacataaggcgggggctcaggccccggaagagccctatcttcccatccacctgcacaatgtacttggcataggttgtagaagctcggcaggta

SEQ ID NO: 16 - DNA, 890 bp, Mus musculus
tagatgcatgctcgagcggccgcccttttttttttttttttttccaaatcaccaccaatacatttattcgaggagatgggtctatcttaccacgaggggaggactagatgtcggcctatgtaacctgtgcgtattcgcacccagcacagtgactgaaccctcacacctggcgtcaccagcacagacaagcagatgaggggatggtctgaggagaacatgatttcctattcaggagaaggcaccacccttgtataagaaaattagtgttgggaacatagcgccagcctcccatggcccaggtgtgatggcgcccaatttacaaagcaggaagtggggggcgggggtgcttctggctgactggcaggatgagctgggctagaggtgcagggaagccttgccactgagtgacgtttgcctctgcagcctgcctctgcctgagtacaagatggactccagtacctctaggcaggaaggggatgccaccccaacactgctcccccaggcttccccaggtcccaggtgacccacctccaccagcccacatctggacagacacatggcaaatatggacatgaagccaggctgggctggagtcacccttgggcatctgagatgatgggtcctcaccaggggtaggctaagaccccataaatgtttggtcgggcagcttaggttactccagggcaaagcatgaccccgatgacacccggcggaaaagcaggctggagccgcggaagagctggccctgcacactcaggtacttccagcagtggatccaggacttgaacacaggggaatacggaggagtccagcccgcagcccacagttgttcactgccatgagatctccaacgagcaggaaggggtaggtcagcatgctcactgcaatcc

SEQ ID NO: 17 - DNA, 1836 bp, Mus musculus
ggtaccgagctcggatccactagtaacggccgccagtgtgctggaaaggggcttcggacccggaagtggcgccttgggctcccggcggcgccgcggggatggcgggagccggagctggtgcaggagctcggggcggcgcgccggccggagtcgaggcccgcgctcgggacccgccacccgcgcaccgcgcgcaccctcgccatcctcggcccgcggctcagccgtcggcgcgcaggatggacggcggcccgggcgccccgggctccggggacaacgccccgaccaccgaggcgctgttcgtggcgctgggcgcgggcgtgacggctctcagtcacccgctgctctacgtgaagctgctgatccaggtgggtcatgagccgatgccccccacccttgggaccaatgtgctggggaggaaggtcctctacctgccgagcttcttcacctatgccaagtacattgtgcaggtggatgggaagatagggctcttccggggcctgagcccccgccttatgtccaacgccttgtccactgtgacccgcggcagcatgaagaaggttttccctccagatgagatggagcaggtttccaacaaggacgacatgaagacctcactcaagaaagttgtgaaggagacatcgtatgagatgatgatgcagtgtgtatcgcgaatgctggcccatcccttacacgtgatctcgatgcgatgcatggtgcagtttgtgggacgggaggccaagtacagtggtgtgctgagttctattgggaagatcttcaaggaagaggggctgctgggattcttcgttggcttaatccctcacctcctgggcgatgtggttttcttgtggggctgtaacctgctggcccacttcatcaatgcctacttggtggacgacagctttagccaggccctggccatccggagctacaccaagtttgtgatggggattgcagtgagcatgctgacctaccccttcctgctcgttggagatctcatggcagtgaacaactgtgggctgcgggctggactccctccgtattcccctgtgttcaagtcctggatccactgctggaagtacctgagtgtgcagggccagctcttccgcggctccagcctgcttttccgccgggtgtcatcggggtcatgctttgccctggagtaacctaagctgcccgaccaaacatttatggggtcttagcctacccctggtgaggacccatcatctcagatgcccaagggtgactccagcccagcctggcttcatgtccatatttgccatgtgtctgtccagatgtgggctggtggaggtgggtcacctgggacctggggaagcctgggggagcagtgttggggtggcatccccttcctgcctagaggtactggagtccatcttgtactcaggcagaggcaggctgcagaggcaaacgtcactcagtggcaaggcttccctgcacctctagcccagctcatcctgccagtcagccagaagcacccccgccccccacttcctgctttgtaaattgggcgccatcacacctgggccatgggaggctggcgctatgttcccaacactaattttcttatacaagggtggtgccttctcctgaataggaaatcatgttctcctcagaccatcccctcatctgcttgtctgtgctggtgacgccaggtgtgagggttcagtcactgtgctgggtgcgaatacgcacaggttacataggccgacatctagtcctcccctcgtggtaagatagacccatctcctcgaataaatgtattggtggtgatttggaaaaaaaaaaaaaaaaaagggcggccgctcgagcatgcatcta

SEQ ID NO: 18 - DNA, 747 bp, Mus musculus
  misc_feature (464)..(730): n = any nucleotide
agctccgcccctgctactggaccatggagactgtggcccagtagagaccttagtgtgaggctttcaggggcggcggccatggaggccgtgctgaacgagctggtgtctgtggaggatctgaagaattttgaaaggaaatttcagtctgagcaggcagctggttctgtgtccaagagcacgcaatttgaatatgcctggtgcctggttcgaagcaaatacaatgaggacatccgcagaggcatcgtgctgctggaggagctgttgcccaaagggagcaaagaggaacagcgggactatgtcttctacctggccgtgggcaactaccggctcaaggaatatgaaaaggctctaaagtatgtgcgagggctgttgcagactgagccccagaacaaccaggccaaggagctggaacgcctgattgataaggccatgaagaaagatggactggtaggcatggccatcgntggtggcatggccctgggcgtggcaggcctggctggactcattggactggctgtcttccaagtncaaatcctgaaggcagnctnacctgctctnttgcccgggacgcctaggagcctgggggacactggaagaggggcctgtccatactaccatcgccttccttttttctgcacccctgtagtctacctttacagcttcatgacccccagccttttaanncctncacctggtngtttaaccctntcattcctttgcaatgagtgnnaaataaaaattggcccc

SEQ ID NO: 19 - DNA, 761 bp, Mus musculus
  misc_feature (41)..(761): n = any nucleotide
cccgagccacggcccaattttatttacactcattgcaaagnatgacagggttaaacgacaggtgcaggggctaanaggctgggggtcatggagactgtagaggtagactacaggggtgcagganaaaagggaaggcgatggtgaggatggacaggcccctnttccagngtcccccaggctcctaggcgtcccggggcagagagcaggtgaggctgccttcaggatttggacttggagacagccagtccaatgagtccagccaggcctgccacgcccagggccatgccaccaacgatggccatgcctaccagtccatctttcttcatggccttatcaatcaggcgttccagctccttggcctggttgttctggggctnagtntgnaacagccctcgcacatactttagagccttttcatattccttgagccggnagttgcccacggccaggtagaagacatagtcccgctgttcctctttgctccctttgggcaacagntcctccagcagcacgatgcctctgcggatgtcctcattgaatttgcttcgaaccaggcaccaggcatattcaaattgcgtgctcttggacacagaaccagntgcctgctnagactgaaatttcctttcaaaattnttcagatccttcacagacaccagctcgttcagcacggnctccatggccgccgcccntgaaagccttacactaaggnctntactgggccacaagtttccatggtccagagcangggccggagcttnnan

SEQ ID NO: 20 - DNA, 901 bp, Mus musculus
  misc_feature (505)..(896): n = any nucleotide
agattgacttgggcactgacatggttcctgccatctccctggcctacgagcaagctgagagcgacatcatgaagaggcagcccagaaaccccaaaacggacaaacttgtgaacgagcgtctgatcagcatggcctatggacagatcggtatgatccaggccctgggaggcttcttcacttactttgtgattctggctgagaacggtttcctgccctttcacctgttgggcatccgagagacctgggatgaccgctgggtcaacgatgtggaggacagctacgggcagcagtggacctacgagcagaggaagatcgtggagttcacctgccatacagcgttctttgtcagtattgtggtagtgcagtgggccgacttggtcatctgcaagaccagaaggaattctgtcttccagcagggaatgaagaacaagatcttgatatttggcctctttgaagagacagcccttgctgctttcttatcctactgccccgggatgggggcagnccttaggatgtatccctcaaacctacatggtgggtctgtgcctttccctacttcccttcttaatcttttgtgtatgacgagggtgcgggaagcttaattattaaggcgggngccccttggcnggnttgggttgggaggaanggaggaccctacttacttaggccccacttgccccttggnacgcccgggggggaaacaattttgtgnccaacaacaccttgnaaccccaacccccttaccccccctttttttggggggacctttcaaagggtttttgggaggccttngggaaactttttaccccttgggnnggggaaaaggcaccccaaaaccntttgttggggggattgccaaaaccnttccttggaaatggaaaanaattgttancttntaacc

SEQ ID NO: 21 - DNA, 472 bp, Mus musculus
  misc_feature (133)..(465): n = any nucleotide
ccgttataatagccatctttatttgtaaaaatccagatataaaacgtaatctttcagtctttccaggttttccttttttacaaaaacaaaaaggcacgtataaaccttgcccgctgtcgtccccgtacacggngtttctcaggcagccctcccccccgccccgccccccgttacagctacatgcttcattccaggacgtctgcatccccacatgcttnggngctttcctaccagggtagagttccgagctccaagacttgaagtacacaaagagggggtaggggngggngcagngngtggcacaatgttccacggcgtgcagggcagagggctagtagtaggtctccttctccacccagccgcagggcgccgcctgataatgagcttccgcacctngnatacacaaagatgagaagggagtaggggaaggcacagaaccaccatgtaggtttgaggggatacatnctaaggg

SEQ ID NO: 22 - DNA, 621 bp, Mus musculus
  misc_feature (436)..(593): n = any nucleotide
agccggaggccgagcccagtcgccagctcctgctctgctcctctcccgcctgccgccgcgctgcacgcctcgagcactccctcggccccggcggggaccggggaccccgcagctaccgccatgctgccagtgctctacaccggcctggcggggctgctgctgctgcctctgctgctcacctgctgctgcccctacctcctccaagatgtgcggtacttcctgcggctggccaacatggcccggcgggtgcgcagctaccggcagcggcgacccgtgcgtaccatcctgcgggccttcctggaacaagcgcgcaagaccccacacaagcccttcctgctgttccgagacgagacgctcacctacgcccaggtggaccggcgcagcaaccaagtggcgcgggcgctgcacgatcaactgggcctacgacagggggatngcgtagccctcttcatgggcaatgagccggcctacgtgtggatctggctgggactgctcaaactgggctgtcccatggcgtgcctcaactacaacattcgtgccaagtctctgctgcactgctttcaatgctgcggggcgaaggtgctgctggcctncccagatctacaagaagctgtggaggag

SEQ ID NO: 23 - DNA, 571 bp, Mus musculus
  misc_feature (212)..(454): n = any nucleotide
agtgttaatatagtttattatgtctttaaaaaaataaggccctctctccaagaagcttagtttgcaaggacaaatggcaggtgcacattgaaaaataattgtttctaaatctttttacttgcaaaggttcaggtgtaatttaaaaaaaaaacaaacaaactatccttttaatgaataatttcctaaaaataaaatcgcaccttatagccttnaatcaagttaaagttggattctacgtatgaagtggctctgcgaggtctatcgagtttctttctggaaatgtcatgagctaaaccaccagggaatattcagagcttnagagttttatcaattatggcattataaatgttctcagtcatgggcacaaatgttttctctgcatcatccatgaaatacaaggtatctttgatgactgtgggattgaagccctcttccatcagggtcactttgcggngtttaaaagtcccagtgatctcaatggtatcttgtatcctcaggaaccgaggcctcgcgtaactgggcaggtactccgcgatgtgttgaaagagtttctttccattgaactcgtagt

SEQ ID NO: 24 - DNA, 673 bp, Mus musculus
  misc_feature (145)..(409): n = any nucleotide
agcgcgggaggcgcatggcgggcatggcgctggcgcgagcatggaagcagatgtcctggttctactaccagtacctgctggtcactgcgctctacatgctggagccctgggagcgaaccgtgttcaattcgatgctggtttccgnggtggggatggccctgtacactggctacgtcttcatgccccagcacatcatggctattctgcattactttgaaattgtacagtgacgaagatgtgaccaggatccagaggttcctggggaagatctgccttgtgaagttggaatgagacctcatcagatgtaagatgtgctacggatgtccacgtgaccaaccttataaatacaaagactttaaaaaaaacttnatgagtagaacaggaaaaatcatcctggctcatgtgttgngttctttctttttgattttaacagaggctcttatatagtagcttttatctattttaacattgtagtcatttgtactttgatatcagtattttcttaacctttgtgactgtttcaatattatccagtgaaagcttttcttaatgtaactttgagtacatctcaattgccttctatttttaaaacctaaggtcattagttgggctttactggtcttgctatcatatggcatatacatctgcctggatatatttctactcttgacca

SEQ ID NO: 25 - DNA, 654 bp, Mus musculus
  misc_feature (32)..(606)
  misc_feature (32)..(32): n is a, c, g, or t
acatttttctttgaatttaatgagtttacatnaaaaaaaagtagtcattttacatntaaggaataaaaaccgttttaaaaaaaatacaaagagtgaaaggatttttaagcaagtttacatttcttttggttatggttctgcacaattcatctcattgngtctttatnacaacgtgcaaatgcatttnacaacgcctgttacaacatnaaattaactnttgagcgtatacagggtcaatactgcctnagaggatctgataagccttctatgaaaagctncacagtgtatntnagcatatgtcatacaagccggccaccaatcaccaatnacaggaatcatnaaagttggttggaaataagtccacataagaatttaatatntaaaaggngaaatgttccttgtattaatgttagcaagatctttactttttcattactaagaaacactttaatagttttagagcaaaagctgttaagagtctagggagctaaaaccgtactcctgagttcaagcaagcagataaatcttttgtaagtagttctnaaagtatcctccctcccgtccccaaattctgtattgnttcttacaaaactttggtcaagagtngaaatatatccaggcagatgtatatgccatatgatagcaagaacagta

SEQ ID NO: 26 - DNA, 1282 bp, Mus musculus
  misc_feature (925)..(1272): n = any nucleotide
agcgcgggaggcgcatggcgggcatggcgctggcgcgagcatggaagcagatgtcctggttctactaccagtacctgctggtcactgcgctctacatgctggagccctgggagcgaaccgtgttcaattcgatgctggtttccgtggtggggatggccctgtacactggctacgtcttcatgccccagcacatcatggctattctgcattactttgaaattgtacagtgacgaagatgtgaccaggatccagaggttcctggggaagatctgccttgtgaagttggaatgagacctcatcagatgtaagatgtgctacggatgtccacgtgaccaaccttataaatacaaagactttaaaaaaaacttcatgagtagaacaggaaaaatcatcctggctcatgtgttgwgttctttctttttgattttaacagaggctcttatatagtagcttttatctattttaacattgtagtcatttgtactttgatatcagtattttcttaacctttgtgactgtttcaatattatccagtgaaagcttttcttaatgtaactttgagtacatctcaattgccttctatttttaaaacctaaggtcattagttgggctttactgktcttgctatcatatggcatatacatctgcctggatatatttctactcttgaccaaagttttgtaagaamcaatacagaatttggggacgggagggaggatactttgagaactacttacaaaagatttatctgcttgcttgaactcaggagtacggttttagctccctagactcttaacagcttttgctctaaaactattaaagtgtttcttagtaatgaaaaagtaaagatcttgctaacattaatacaaggaacatttcaccttttagatattaaattcttatgtggacttatttccaaccaactttnatgattcctgtgattggtgattggtggccggcttgtatgacatatgctragatacactgtggagcttttcatagaaggcttatcagatcctctraggcagtattgaccctgtatacgctcaaragttaatttratgttgtaacaggcgttgtraaatgcatttgcacgttgtgataaagacncaatgagatgaattgtgcagaaccataaccaaaagaaatgtaaacttgcttaaaaatcctttcactctttgtattttttttaaaacggtttttattccttaratgtaaaatgactacttttttttratgtaaactcattaaattcaaagaaaaatgtaaaaaaaaaaanccn

SEQ ID NO: 27 - DNA, 774 bp, Mus musculus
  misc_feature (39)..(774): n = any nucleotide
agcgagtttgcagacttcttgtgcgcagctagccgcctnaggtgttngaaccatgaatcttttactcctttnggctgtcctctgcttgggaacagccttagctactccaaaatttgatnaaacctttagtgcanagtggcaccagtggaagtcnacgcacagaagactgtatggcacgaatgaggaanagtggagganagcgatatgggagaagaacatgagaatgatccagctacacaacggggaatacagcaacgggcagnacggcttttccatggagatgaacgcctttggtgacatgaccaatgaggaattcaggcaggnggngaatggctatcgccaccagaagcacaagaaggggaggctttttcaggaaccgctgatgcttaagatccccaagtctgnggactggagagaaaagggttgngtgactcctgtgaagaaccagggccagngcgggtctngnngggcgtttagcgcatcgggttgcctagaaggacagatgttccttaagaccggcaaactgatctnactgagtnaacagaaccttgtggactgttctcacgctcaaggcaatcagggctgtaacggaggcctgatggattttgctttccagtacattaaggaaaatggaggtctgnactcggaggagtcttacccctatgaagcaaaggacnggatcttgtnaatacagagncgagttcgctgtggctaatgacacagggttccgtggatntnccttagccaggagaaagcctcatgaagctgtggctn

SEQ ID NO: 28 - DNA, 723 bp, Mus musculus
  misc_feature (70)..(723): n = any nucleotide
ttacacacacactgagctaaaatttatttttaagaggtaaagaaagttttatacaacctttatataaaanaaatattnnagactnagaattaagcactaagttttcaatattataaagntgtttataanaggagttttaagtagnggtaacatttaacccatgtaaaaatggcaacagaattnaaatnatganttggatcctnaatgattnaagnaccanggttngantnaataagggttnggtcagttttaagctgaattccttnggacatagagnccataagtcctcattancgctaccnatnaattnangacaggatagntggncgcggnggcaagtccacagaggtngncccggtcttnggctatttngatgtagccttccataccccattcacttccccagctgntcttgacaagccaatatttattcttatttgaatctgntccttnatagccatagccnancaacagaaccccatggtcgaggatcttgctgctacagtngggttnatagtagatgcctgaactatagaactggagagacggatggcttgcgtccatagcaacagaaataggccccacagtcgcacagccttcatgagggctttctcttgctgagggatatcnacgaaccctgtgtcattagccacagggaactnggctctgtatttacnagaancggcctttgcttcanagggngtaaggacttctccgngtccn

SEQ ID NO: 29 - DNA, 341 bp, Mus musculus
  misc_feature (25)..(282): n = any nucleotide
agatggcttccagcggagtgactgngagcgccgncgggtcggccagcgaggcctnagaggttccagacaacgtgggagactggctccgcggcgtcttccgcttcgccaccgatcgaaacgacttccggaggaacttgatccttaatttgggactctttgctgcgggagtctggctggccaggaacttgagtgacattgatttgatggcccctnagccaggggtgtagccagagaatggaactcctgtgtattcagactttccaaagacagcctactgtctgngaccacaagatcctacctgagtggcagctgaagttgactccctctccttgcctgaaccc

SEQ ID NO: 30 - DNA, 156 bp, Mus musculus
  misc_feature (4)..(138): n = any nucleotide
atgngcaggctttatttgaaatctttttcaagaaccattattactcttnaggacaagggcaaggaccatcttctgcagaaagtngggaactgcacacagaaccgtgcagaggcaacatnttagccgacactgggggangggggggcagaggggggg

SEQ ID NO: 31 - DNA, 513 bp, Mus musculus
tgrkatggcttccagcggagtgackgtgagcgccgccgggtcggccagcgaggcctcagaggttccagacaacgtgggagactggctccgcggygtcttccgcttcgccaccgatcgaaacgacttccggaggaacttgatcctyaatttgggactctttgctgcgggagtctggctggccaggaacttgagtgacattgatttgatggcccctcagccaggggtgtagccagagaatggaactcctgtgtattcagactttccaaagacagcctactgtctgtgaccacaagatcctacctgagtggcagctgaagttgactccctctccttgcctgaacccccccycactstctcccccatcccccagtgtcggctragatgttgcctctgcacggttctgtgtgcagttcccaactttctgcagaagatggtccttgcccttgtcctgaagagtartaatggttcttgaaaaagatttcaaataaagcctgcacataaaaaaaaaagaga

SEQ ID NO: 32 - DNA, 826 bp, Mus musculus
taccgagcccggatccctagtaacggccgccagtgtgctggaaagctcgtcatggcgacggagcagaggcctttccacctggtggtgttcggcgcctctggcttcaccggccagttcgtgacggaggaggtggcccgggagcagatagcctcggagcagagctcccgcctgccctgggccgtggcgggtcgctccaaggagaagctgcagcaagtgctggagaaggctgcccagaaactgggaagaccatcactatcatctgaagttggagtcataatctgtgatatcagtaatccagcctcacttgatgaaatggctaaacaggcaaagcttgtcctcaactgcgtaggaccgtatcgattttatggagaacctgtagtaaaagcatgtattgaaaatggaacaagttgtattgacatctgtggggaacctcagtttctggaactaatgcatgcgaagtatcatgagaaagctgcagagaagggggtttatatcattggaagcagtggctttgactccatcccagcagatctaggagtgctatacaccaggaaccagatgaacggtactttgactgctgtagaaagcttcctgacaataaatacaggacctgaggggttgtgtattcatgatggaacctggaagccggcaatttatggttttggcgataagggtagtttaagaaaactacggagtgtatcatgtctgaaacctgtcccaattgttggtacaaagttgaaaagaaggtggccagtcagctattgtagagagctgaactcgtattccattccttttttgggatctgatatatctggctggagcaaaa

SEQ ID NO: 33 - DNA, 374 bp, Mus musculus
  misc_feature (2)..(367): n = any nucleotide
angngnaataangngangggntaaaagagnaannaantanagtgagagagggaatgnaggnnaaatatacncctnanccntatattggaaannnacctnatattnnaaacnnnnctannntnatnnanatnanatacnnatnaatnanacnnaannannannntactancaaacaacnannnnaatnnnnnaannnaaanctttannacanaatcnanncnnanaaacnatttcttaancancanataannaaannnncnaananaantnacanacaananancnaattanntcatnnancacctaannncnaaanntnnntnantncnnaattnncnnnnatanttatcnntactanttatctnancactatc

SEQ ID NO: 34 - DNA, 1455 bp, Mus musculus
taccgagcccggatccctagtaacggccgccagtgtgctggaaagctcgtcatggcgacggagcagaggcctttccacctggtggtgttcggcgcctctggcttcaccggccagttcgtgacggaggaggtggcccgggagcagatagcctcggagcagagctcccgcctgccctgggccgtggcgggtcgctccaaggagaagctgcagcaagtgctggagaaggctgcccagaaactgggaagaccatcactatcatctgaagttggagtcataatctgtgatatcagtaatccagcctcacttgatgaaatggctaaacaggcaaagcttgtcctcaactgcgtaggaccgtatcgattttatggagaacctgtagtaaaagcatgtattgaaaatggaacaagttgtattgacatctgtggggaacctcagtttctggaactaatgcatgcgaagtatcatgagaaagctgcagagaagggggtttatatcattggaagcagtggctttgactccatcccagcagatctaggagtgctatacaccaggaaccagatgaacggtactttgactgctgtagaaagcttcctgacaataaatacaggacctgaggggttgtgtattcatgatggaacctggaagccggcaatttatggttttggcgataagggtagtttaagaaaactacggagtgtatcatgtctgaaacctgtcccaattgttggtacaaagttgaaaagaaggtggccagtcagctattgtagagagctgaactcgtattccattccttttttgggatctgatatatctgttgtgaaaaggactcagcgttacttacatgaaaatttagaggactcaccagttcagtatgctgcttatgtgacggtgggaggcatcacctctgtgattaagctgatgtttgcaggactgttctttttattctttgtgaagtttagcattggaagacaacttctcataaaattcccatggctcttttcctttggctatttttcaaaacaaggtccaacacaaaaacagatggatgagacatcatttacaatgacattctttggtcaaggatacagccatggcacttgtgttgaaaagaacaaaccaaatatccgaatctgcactcaagtgaagggaccagaggctggctacgtggctactcccatagccatggttcaggctgccatgacttttctgagtgacgcctctgaccttccaaaagggggcggtgtctttacacctggagcagctttctccagaacaaagttgattgacagactcaacaaacatggcattgaatttaagtgtcattagcagctccgaagtctaaacgtttgaagactaaccgaatcataaaatgcacaaaccgcgtctgtatttggatatgtgaaattcttctataagcctatctgactgtatgtggactgtcaagttataaaatat

SEQ ID NO: 35 - DNA, 464 bp, Mus musculus
  misc_feature (18)..(443): n = any nucleotide
aaaggaaaaacacagctnagcagatccaggcactaaagagagctagctgcaagcaggagcagtcaagaatctgnggtcagaagtactggagngggccagcagggccagctttttctaccatggcagcccaaggctacggctactatcgcactgtcatattngcggccatgttnggaggctacagcctgtactatttcaaccgcaaaaccttctcctttgtcatgccctccttggnggatgagatcgctctggacaaggacgattnggggctnatnacaagcagccagtcggcagcctacgccatcagcaagttngngagcggngntctgtcagatcagatgagcgcccgctggctcttctcctctgggctgctcctggtnggtctggtcaacgtagtcttcttatggngctccacagngtcagccttagctgctcttnggttntcttaatggtcctggcacagg

SEQ ID NO: 36 - DNA, 388 bp, Mus musculus
  misc_feature (1)..(370): n = any nucleotide
nagnatcaaanctagcttnanngatctanacaagncggntngccctctatattcttccctttngncccagggnattcanganagaggaggactcctctcctcccttagggacngaggnannggcaacaatttngcccccngccaagaagcctnggggnacagcagaaaccacaggccattaatactcactaggagatcaggacctaggagaagaagaagggtataggagacactctgaantnaggagnngccngncngccagaaggnagaaacagaagcngaaggagtctagaaccaaacatcatcattttaaatagagcangggaagggagnngggacctnantagccacnggaaacttgcagcaccanggaggttcagagagacag

SEQ ID NO: 37 - DNA, 453 bp, Mus musculus
atggctgcgctcttgctgagtgctgctcctttgggaaccacagctaaggaggagatggagcggttctggaagaagaacacgagttcaaaccgtcctctgtctccccatttgactatctacaaatggtctcttcctatggcactgtccgtttgccaccgaggctctggaatagccttgagtggaggggtctctctttttggcctgtcggcactggtgcttcctgggaactttgagtcgtatttgatgtttgtgaagtccctgtgtttggggccaacactgatctactcggctaagtttgtgcttgtcttcccgctcatgtaccactcactgaatgggatccgacacttgctatgggacctaggaaaaggcctggcaataccccaggtctggctgtctggagtggcggtcgtggttcttgctgtgttgtcctctggcgggctggccgccctgtga

SEQ ID NO: 38 - DNA, 888 bp, Mus musculus
cgagctcggatccactagtaacggccgccagtgtgctggaaaggtgacagaggggaacaagatggcggcgccaaaggggaagctttgggtccaggcccaactggggctcccgccgctgctgctgttgactatggcgctggccggaggctcggggactgcagcggccgaagcctttgactcggtcctgggagacacagcgtcctgtcaccgggcctgtcagctgacctaccccttgcacacctacccgaaggaagaggagttatacgcatgccagagaggctgcaggctgttttcaatttgccagtttgtggatgatgggcttgatttaaatcggaccaagctggaatgtgaatctgcgtgcacagaagcatattcccaacctgatgagcagtatgcttgtcatcttggctgccaggatcagttgccatttgctgaactgagacaagaacaactcatgtccctgatgccaagaatgcatctcctcttccctctgactctggtgaggtcgttctggagtgacatgatggactctgcacagagcttcataacctcttcatggactttttatcttcaagccgatgacggaaaaatagttatattccagtctaagccagaaattcagtatgcaccgcagttggagcaggagcctacaaacttgagagaatcatctttaagcaaaatgtcctatctgcagatgagaaactcacaagcacacaggaactaccttgaagaggaagaaagcgatggctttttaagatgtctatctcttaactctggatggattttaaccacaacccttgtcctctcggtgatggtgttgctctggatctgttgtgcagctgttgctacagctgtagaacagtatgttccccctgagaagctgagtat

SEQ ID NO: 39 - DNA, 440 bp, Mus musculus
  misc_feature (15)..(438): n = any nucleotide
cagaacataattatngaaatagattttaangatttcaattnaatacaactgaaaangtagagncattaaataacatttctgctataatccagaggacagtttggaggccatttncgggcagaagcatcacaccctaaggnttcggntattaagtnagangactgacggtgngcangncaggggnggagccacacntgatcagctcatagaanntcggtgaanagaggaaancanancacacccaantgcactanctaantantnacagatattagnntnaatctcanntacancccaatgnccatcttaaantgactagaaannnccaggtnaancttacancnaaatanngcccttcatnganntatggtaacctnctatntngcattttatagcngtnttccttaanggcctatnnttcnanatgncacncatntnta

SEQ ID NO: 40 - DNA, 875 bp, Mus musculus
tgcatgctcgagcggccgcccttttttttttttttttttttcagaacataattattcaaatagattttaatgatttcaattcaatacaactgaaaatgtagtgtcattaaataacatttctgctataatccagaggacagtttggaggccatttccgggcagaagcatcacaccctaaggtttcggttattaagtgagatgactgacggggggcatggcaggggcggggccacaatgatcagctcatagaatttggcgaagagaggaaaaaaaccaaaaccaatgcaactaactaagtagctacagatattagtaaaataaaaatacaacccaatgtccatcttaaatgactagaaaaatacaggtaaagtcacagcaaataaagtcttcacagagtttggtaactttatttgcattttatagtgatttcttaaggcctatgtccaatgaaaccatcttaaaaagctctatgaggaatggaagtttatgtgtccacgactcttttaaaaagcttagatttctgagtgagcaaggttcaccttggtgggcaggggccctgcctcctcatgttcttcagtctgagacctaacaatcacaagagaaggagctgggtatctgctcagcttttgttcattcataaattccaagtcaccatagatactcagcttctcagggggaacatactgttctacagctgtagcaacagctgcacaacagatccagagcaacaccatcaccgagaggacaagggttgtggttaaaatccatccagagttaagagatagacatcttaaaaagccatcgctttcttcctcttcaaggtagttcctgtgtgcttgtgagtttctcatctgcagataggacattttgcttaa

SEQ ID NO: 41 - DNA, 1545 bp, Mus musculus
cgagctcggatccactagtaacggccgccagtgtgctggaaaggtgacagaggggaacaagatggcggcgccaaaggggaagctttgggtccaggcccaactggggctcccgccgctgctgctgttgactatggcgctggccggaggctcggggactgcagcggccgaagcctttgactcggtcctgggagacacagcgtcctgtcaccgggcctgtcagctgacctaccccttgcacacctacccgaaggaagaggagttatacgcatgccagagaggctgcaggctgttttcaatttgccagtttgtggatgatgggcttgatttaaatcggaccaagctggaatgtgaatctgcgtgcacagaagcatattcccaacctgatgagcagtatgcttgtcatcttggctgccaggatcagttgccatttgctgaactgagacaagaacaactcatgtccctgatgccaagaatgcatctcctcttccctctgactctggtgaggtcgttctggagtgacatgatggactctgcacagagcttcataacctcttcatggactttttatcttcaagccgatgacggaaaaatagttatattccagtctaagccagaaattcagtatgcaccgcagttggagcaggagcctacaaacttgagagaatcatctttaagcaaaatgtcctatctgcagatgagaaactcacaagcacacaggaactaccttgaagaggaagaaagcgatggctttttaagatgtctatctcttaactctggatggattttaaccacaacccttgtcctctcggtgatggtgttgctctggatctgttgtgcagctgttgctacagctgtagaacagtatgttccccctgagaagctgagtatctatggtgacttggaatttatgaatgaacaaaagctgagcagatacccagctccttctcttgtgattgttaggtctcagactgaagaacatgaggaggcagggcccctgcccaccaaggtgaaccttgctcactcagaaatctaagctttttaaaagagtcgtggacacataaacttccattcctcatagagctttttaagatggtttcattggacataggccttaagaaatcactataaaatgcaaataaagttaccaaactctgtgaagactttatttgctgtgactttacctgtatttttctagtcatttaagatggacattgggttgtatttttattttactaatatctgtagctacttagttagttgcattggttttggtttttttcctctcttcgccaaattctatgagctgatcattgtggccccgcccctgccatgccccccgtcagtcatctcacttaataaccgaaaccttagggtgtgatgcttctgcccggaaatggcctccaaactgtcctctggattatagcagaaatgttatttaatgacactacattttcagttgtattgaattgaaatcattaaaatctatttgaataattatgttctgaaaaaaaaaaaaaaaaaaaagggcggccgctcgagcatgca

SEQ ID NO: 42 - DNA, 384 bp, Mus musculus
  misc_feature (293)..(312): n = any nucleotide
aaaggtacgaagctagggaagatattcgcgtggctaaatctgcacgtggaaggagcattaacttggccctttcttatagaggacggcaggccttgaaagccattggtctggaagatcagatcgtttccaaaggtgtgcccatgaaagccagaatgatccactctctttctggaaagaagtctgcaattccctatgggaacaagtcacagtatatcctttcaataagcagagaaaacttaaacaaggacctgctgactgccgaggagtcctatgccaatgcgaaggtgcacttnggccacaagctgtcgaaangcattccggaggaaggggtactcacagagctcggacctgacaaggttccccgagatgtcacatgtgaccttg

SEQ ID NO: 43 - DNA, 488 bp, Mus musculus
  misc_feature (2)..(482): n = any nucleotide
antttttttttttttttttttttttttttttttttttttttttttttttnaaaanggnanaangntttngnaaantttngnnggnnngggnaaanaaaccnagggncnnangncggnaaaaaaagnnttttnccnnagggnnnnannnntnaccngnaaannaangnntttnntttncntacccctnaaaaanaaannggnaaancccanggnngntttnganannanggnaaannccaantngnttaannttaanttccnngggttnggnttttnnaggnaannaanangggnnntntnaaaanngnannangaaaanaantttaangggtccnannnacccnnttnggaaaaaannaaatngnaangggnanttgggcnntggncttngnngacnaagtnaantttnggntnngnccggggggnannnanggnnccccttttgnaagnntnaangggnnaaaanggggncccannntttnggaaaa

SEQ ID NO: 44 - DNA, 520 bp, Mus musculus
  misc_feature (16)..(494): n = any nucleotide
aaagcgactgcacagngaagccctctgttacctgtgtcgatcaagacctnaaaccccagaggaacttcgtcatcaacatgacttgcaggtttngctggcagcttcctgaaacagactacgagtgttcaaattccaccacctgcatgaccgnggctngccctcggcagcgctatttcgccaactgcaccgngcgtgaccacattcactgcctgggcaaccggactttccctaagctgctgtactgcaactggacaggnggctacaagnggtcgacagccctggctctnagcatcaccctnggggggntaggagccgatcgcttctacctgggccagnggcgagaaggcctcggcaagctcttcagctttggcggcctgggaatatggaccctaatcgatgtctngctgatnggagtaggctatgngggaccagcggatggctctttgtacatttagccgaggttatgtgcttcagagagcagngtagagtcctgngtgtggagatggatgcggagtgagag

SEQ ID NO: 45 - DNA, 1033 bp, Mus musculus
  misc_feature (4)..(1025): n = any nucleotide
cagngttcgatttctttattttaccttcatcaaggcaagccaagtacagatgctgtacattaaaaacataaatccccctntcacaccgcatccatntccacacacaggactctacactgctctctgaagcacataacctcggctaaatgtacaaagagccatccgctggtcccacatagccaantccaatcagcaagacatcgattagggtccatattcccaggccgccaaagctgaagagcttgccgaggccttctcgccactggcccaggtagaagcgatcggntccaaaccccccangggtgatgctgagagccagggctgtcgaccacttgnagccacctgtccagttgcagnacagcagcttagggaaagtccggttgcccaggcagtgaatgtggncacgcacggtgcaagttggcgaaatagcgctgccgagggcaagccacggcatgcaggtggtggaatttgaacactcgnagtcngtttcaggaagctgcagcaaaactgcaggcatgttgatgacnaagtccttggngattgaggcttgacgacacagntaacagagggcttacttngcaagccgntttcagcacaattggccgcncggaactaggggaanccgaggtttgggacccaagcttggggnctcccttaaaggggnggtccgaaatnaaatttccganaaggccnggnaanccaagnggggnncttntaggttagccccgaagnggcntctgcttaanntaggacctttccaccggaaacgcctaacggcccaattggggnnaaaagggncccgngttgtacccacaattttgnaaagncccntcgattggggccaaacaanctcccatggcntcaaangggggaatgnaaacccggggtaaancgttcccccccatgggntgcnaacngnaacaggnaaggngnntaaacnngcnnccccncgagcccaaggntctggcaaagngngggcnaaaccattcccnaangggatnaaaaaactngtcccccaggggtcnaaacccct

SEQ ID NO: 46 - DNA, 586 bp, Mus musculus
gcaacaattcgagctgctgtgacagaggggaacaagatggcggcgccaaaggggaagctttgggtccaggcccaactggggctcccgccgctgctgctgttgactatggcgctggccggaggctcggggactgcagcggccgaagcctttgactcggtcctgggagacacagcgtcctgtcaccgggcctgtcagctgacctaccccttgcacacctacccgaaggaagaggagttatacgcatgccagagaggctgcaggctgttttcaatttgccagtttgtggatgatgggcttgatttaaatcggaccaagctggaatgtgaatctgcgtgcacagaagcatattcccaacctgatgagcagtatgcttgtcatcttggctgccaggatcagttgccatttgctgaactgagacaagaacaactcatgtccctgatgccaagaatgcatctcctcttccctctgactctggtgaggtcgttctggagtgacatgatggactctgcacagagcttcataacctcttcatggactttttatcttcaagccgatgacggaaaaatagttatattccagtctaagc

SEQ ID NO: 47 - protein, 183 aa, Mus musculus
MAAPKGKLWVQAQLGLPPLLLLTMALAGGSGTAAAEAFDSVLGDTASCHRACQLTYPLHTYPKEEELYACQRGCRLFSICQFVDDGLDLNRTKLECESACTEAYSQPDEQYACHLGCQDQLPFAELRQEQLMSLMPRMHLLFPLTLVRSFWSDMMDSAQSFITSSWTFYLQADDGKIVIFQSK

SEQ ID NO: 48 - DNA, 203 bp, Mus musculus
  misc_feature (35)..(189): n = any nucleotide
aaagctggctggaatcctggtaggagccgcagtgngnggccatgcctcagataggttngggcgcagaagggtgctgacctggagctatcttcnggngtccgngtccggcacagtagctgccttcatgcccaccttccccctctactgnctgntncgtttcctgctggcctctgcagnggcaggagttangatgaacacagcca

SEQ ID NO: 49 - DNA, 187 bp, Mus musculus
  n = any nucleotide (15)..(119): n = any nucleotide
aaaaattatttattngngtgtatcaccgggggcgggnatgtgtccatgcggagctcanaggacaactttgtgaagtctgttgtcacatgagtttcagaaatttaggcccggaggcaggngtctttacccgccgtcgccagccctgccctttcctcctcctcctcctcttcctcctcctcctcctcta

SEQ ID NO: 50 - DNA, 391 bp, Mus musculus
  misc_feature (120)..(376): n = any nucleotide
aaaggggacggagaccttcagcgtggaatctatatccaagaatggaatctgtctggagatgggcccacagcctcagggcgtgctgcgggccgacctgttctcccggatgcgagctctggnggcatccattctggacttcatcgagctcttnaaccaaggcatggacttacccgcctttgagatggatatctacaggaacttgggcagngnggacttcccacgcactgcggnnggngacctggctggcactgngcaccctcaactgcaggaccatgactttgagccactgaggcctggagaacccatcttcaagcttttcagcggagaagangtactgtatnagggggactccattgngtacccctgnnntcattnntgaggctgccctatt

SEQ ID NO: 51 - DNA, 726 bp, Mus musculus
  misc_feature (53)..(724): n = any nucleotide
ggggcacagtctattttatataatgagatcagttgtctggaaagggatatggngtccagagtgagataggggaccctgggcttgggacccattgttcagataggaaaactgagatcaggccaggtatgattngggttagggagtctgggnggagcggggggtcaacctnagcagggcaggcactgngaccctgatcttctcagacttcaggaatgccacgtgcttctcataataggcagcctnattaatgaacacagggtacacaatggagtccccctcatacagtacgtcttctccgctgaaaagcttgaagatgggttcaccaggcctnagnggctcaaagtcatggtcctgcagttgagggtgcacagtgccagccaggtcaccatccgcagngcgtgggaagtccacactgcccaagttcctgtagatatccatctcaaaggcgggtaagtccatgccttggttgaagagctcgatgaagtccagaatggatgccaccagagctcgcatccgggagaacaggtcggcccgcagcacgccctgaggctgtgggcccatctccagacagattccattcttgnatatagattccangctgaaggtctncgtcccctttccagcacactggcggccgatactagaggatccgagctnngtaccaagcttgggctccctaagggagtcnnatantttnnatagccngtaagcagtgggtntctgnta

SEQ ID NO: 52 - DNA, 663 bp, Mus musculus
  misc_feature (31)..(662): n = any nucleotide
aaagggcggcggcagcagctcccgcggctcntgctctgctccgcctcggccccggagcgaggggcggagagccgcgcgctngccttagtccgagccgtcaccctccccgcgntccccgctttnccggcccgcccgaggccgnccgccccgtccccgccgcnccgnagccnggccgtgccccgccgncgncatgngctgcctcggcaacagtnaganccgaggancagcgnanctgaggagaangcgcagcgcgaggncaacaaanagatctgagaagcagctgcagaaggacaagcaggtctaccggntcacgcaccgnctgctgctgcnggnngctggagagnctggcaaaagcacnattgtgaagcanatgaggatcctgcatgntaatgngtntaannganagggcggtaanaggaccccgcaggctgcaaggagcaacanttgatggtnanaaggccactanagngcnngacnttnnanncnatctgnangangccatnnaaaccantgtggccnacatnagcanccttgctgccccctgcnnagntgnccaaccctgcnannnagttcanagntgnnctacattctgngcntcatnaacngtgcncnnacttttcactttccacctnnnttntatnaagccatnnccaaggcttctnt

SEQ ID NO: 53 - DNA, 527 bp, Mus musculus
  misc_feature (88)..(524): n = any nucleotide
gctcatttttaatttttattgattttttaatgctgcacaacacaatatttatttcattttgaatttcatttatttctttatttctgtngctgcttttattttatttactgaaagtgagagggaactttngnggccttttttttctttttcttctgtaggccgccttaagcttactaaatttggaacatctaagcaagctgaagggaagaggggtttttcagaatcactgggggaaaaaggaaaggnngcggagttgatcatgccctatggngggngaccaactgcttgtacaattacgtttcactcttaattaatngngcttaaggctgaattaaatttgggngntcccttcttagagcagctctgnattggcggagatgcatgcgctggatgatgtcacggcagtcgttgaagacacggcggatgttctcagngtccacggcgcaggtaaagtgagggtagcagtngnggcgcccatntccactagcagtgctgnttttcagaaactnatccctnatgaagtnctt

SEQ ID NO: 54 - DNA, 855 bp, Mus musculus
  misc_feature (244)..(854): n = any nucleotide
agcggacgtttgtgccgggacatggccgctgcggatgccgggtccttgagctgagcgcttgctgccggagccaacctctgccgtcaaccgtccgcgcgggctgggcccaggcgccgggacggccaagatccgccgaggaagctgaggcagctatagaacgccgccgcggcgggcgcatggcgtccatcttgctcaggagctgccggggccgggggcctgcccgcctcgcgccacctcgggccgnctccccgcggggtagtctgagggatcgagcttgtctcagctgtaccaggaccctggggttgacgagccgtgagagtgttctgtctcgttgctgtactccagcccaccctgtgtacctctgcttcaaaggtgagcccctnagctgttggactcagaggcctgagtgccagggcaccgcagcaagaacaacatggacacctgcctctgcaaggctggnggttacgggacctcagtaccttcctgtgcgcggctggcactcatcatcttcgctaggagaggactctgtgatagagaagtcccttaagtccttaaaagacaagaataanaagctggangagggcggncccgtgtacagcccggncgcgcaggtggtggtgaggaaatccctngggcagaaggtactggatgagctgaggcactactaccatggctttccgcctgctctgggatngacaccaaagancgcttgnccggatgctctggcgcatncttnatgggncatacgcttaccccgnccgggagccncaggcagnttnttccngaattgtgccggacctnttnccgctaagnggccctttcttgggggnnccnggngggggcccnttaatggna

SEQ ID NO: 55 - DNA, 722 bp, Mus musculus
  misc_feature (21)..(718): n = any nucleotide
cattatggaagtttctgattnattccagacaaaatattanatttgccantaagaatcacctcaaagcaatcactcttgngngtcatgcccctgnggctggcacggcatgatgaaggcagacctgngggccagaagctggcagctctaattcttcacttctgcggcctctttctcagccttctccttggccttctctttctcttctatcttttcttctttctctagngnggccacaatttcagctacctgggnggnagagatctgaacatcttctttgttcaccaagtcgattaccttgacaaggtcatcgatgttgatatngccgnccttattatcatccagggctgaggtcaaactgatgagcttgagctctggaatgngctngatttgcttcatggcgctgatgagctcagngangctgatgacactctcccctgnaggagngctctggctggggcccagnttgccatcctgatgagaggactccagctgtgtgatcaggccatcgatctgcccgatcatctgctgnnctcnctntgacagcctcttgctcgctgcagaatcttctatgnnctttctcctcacccgnctnttnnanggtccttctttgaactcctgtannncctcacntgaggcctggncgccctcttttgagcaggcntccaggcttcctcctttctcccttggcncnggggacnntcccttncgcntcctgcnncttt

SEQ ID NO: 56 - DNA, 814 bp, Mus musculus
  misc_feature (21)..(814): n = any nucleotide
cattatggaagtttctgattnattccanacaaaatattanatttgccactaanaatcacctnaaagcaatcactnttgngngtnatgcccctgnggctggcacggcatgatgaaggcanacctgngggccaaaagctggcagntntaattcttcacttctgcggcctntttntnagccttctccttggccttctctttctcttcnatcttttcttctttctctagngnggccacaatttcagctacctgggnggnananatctgaacatnttctttgttcaccaagtcgattaccttgacaaggtcatcgatgttganattgccgnccttattatcatccagggctgaggtcaaactgatgagcttgngctntggaatgngcttgatttgcttnatggcgctgatgagctcagtgatgctgatgacactntcccctgttggagngctctggctggggcccagcttgccatcctgttgagnggtctccanctgtgtgatcaggccatcgatctgcccgancatctgctgcactcgctttgacagcctcttgctcgctgcaaattcttctatgnacttctcctcacccgtctttgaaagttccttcttgatctcctgtaagtcctcactgtagncctggacgncctctttgagcagntccagctcctccttntncttggtcagggacttcttctgctnctgcagcttggaacaggcgtactgaggatgtnaanctccncttggnanctntttaccctttagacctttcaaacangggccgaatccgnnagganctttgngggcaggaananaaaan

SEQ ID NO: 57 - DNA, 290 bp, Mus musculus
  misc_feature (25)..(285): n = any nucleotide
aaagggcggccatggaccgcttcgngnggaccagnggcctcctggagatcaacgagaccctggttatccagcagcgcggngngcgcgtctacgacggcgaggagaagataaaattngatgccgggactcttcttcttagtacacaccggctgattnggagagaccagaagaataatgagngctgnatggnnattnccctgtcttagatngngntcatcgaggagcaggcagctnnaatcgggaagannnncaaaatngnggntcacctgcacccagttccttttnacaaa

SEQ ID NO: 58 - DNA, 317 bp, Mus musculus
  misc_feature (2)..(314): n = any nucleotide
cnancncattctnggnnaattngggntaatttttnanctnagngtcnngagaccttgnnaaaangcaagntnatngccataaagcatttcaggnncaaaattntnagtnctgggncanaaanaaatttgganaaaaccgaangcnttccanggngcngtntcggaaaaggggnccnattttntttgnanggngcnccntttnttnacccananggncagacnttcccnnangctnggnaannttttnggangtnaaggncccnnttttngaanccgtttcnagggccgnggccncnatttccttttcctngggngtt

SEQ ID NO: 59 - DNA, 457 bp, Mus musculus
  misc_feature (5)..(455): n = any nucleotide
aaagnggggatcctggagacagttctggngcagagttccagtacctctacatccatcattgtcagcatggtctcctccggctngtnggaggngagctccgccattccgatcatcatgggctccaacatcggaacctctgtcaccaacaccattgnggccctgatgcaggcaggggacaggactgacttnaggcgggctttngcaggggcgaccgngcatgactgttttaactggctgtcngttctggncctactgccccnggaggctgccacgggctacctacaccatgtcaccgggctngaggnngcttccttnaacatccnaggnggccgngatgcccccgaccttctcaaagtcatcacagagcccttnanaagactcatcatccagctggacaagtctgngatnaccagcatngccgagggggatnagtncctgaggaatcacagtctcantc

SEQ ID NO: 60 - DNA, 756 bp, Mus musculus
  misc_feature (32)..(754): n = any nucleotide
agggcgccagctgaagacgcgggacttaaagngcgtagccagaacccaggcaccagngtgtccattgtccagaactcatctgaaaaactgccacaggaattgcttctctgctccaggctggtcactgaacaggttgctccaggacctgcagaatgggggcaggctgngtcaaagtcaccaagtatttcctcttcctcttcaactngctgntctttatcctgggngctgagatcctgggcttcggngagnggattcttgcagacaagaacagcttcatttccgtcctacaaacctcatccagctcgctgcaggngggggcttacgtcttcatcggagngggcgccatcaccatagngangggcttcctgggctgtatcggagctgtcaatgaggnccgctgcttgctgggtctgtacttngtcttccttcngctgatcctnatcgcacaggtgaccgtaggggtcctcttctacttcaacgctgacaagcngaagaaggagatggggaacacagngatggacatcattcgcaactacactgccaatgccaccagtagccgcgaggaggcctgggactacgtgcaggcgcaggtcaagngctgtggctgngtcagccactacaactgnacagagaacnaggagctcatgggctttaccnagaccacttacccatgctcctgcgagaaggatcaaggnagaggacnaccagctcattgtgaagaaaggattcttgcgaggctgataacagcactgtngc

SEQ ID NO: 61 - DNA, 292 bp, Mus musculus
  misc_feature (41)..(280): n = any nucleotide
cattataaaccctcctttaataattgattccagagatgagngnatggaacccctcccccaccctgcaaggnacagcctcacccacccttagcgcagaggacaggggacagctgccaagaaacaccagtccagatcctcctctcatccagggtctngngccagacctgagggacccacacccctaagtngtcaggtccctcaccaagaggagcgcaccagaggctacctgggccagtcccccaggaggcccctcagttcagttccctgctgaactgagctnggggggggggag

SEQ ID NO: 62 - DNA, 244 bp, Mus musculus
  misc_feature (12)..(238): n = any nucleotide
aaaggagccctngaaagcgacatggcggntctcttaaagctgggcgntctctgcagnggncaaggagctcgagctctcctactccgaagccgggtggtcagacccgcttatgtgtcagcatttctccaggaccagcctacccaaggacggagtggtacccagcacattcacctgtcaccaagccaccactctggtttcaaggctgcatctctccactggancagtgagagggaagnnngtgccc

SEQ ID NO: 63 - DNA, 202 bp, Mus musculus
  misc_feature (24)..(199): n = any nucleotide
aaaggcagatcgagagggccatgngggccaacgaacaggcgctggcgtctggccngagngagngagttctcatnactgggggcatngaggctacngctggacgtttcacacagaggtatttcggcgcctactctatcgctgcaggngngctcatcngtctgctggagtatccccggngaaagaggaaaaaggggaccancat

SEQ ID NO: 64 - DNA, 103 bp, Mus musculus
  misc_feature (35)..(89): n = any nucleotide
aaagcgcgcagaccgctcctccgctgcagagtcgnttnccngagctnggncgacaaggcngccttcgcagncgggancctgccagccgngaccccagccttcg

SEQ ID NO: 65 - DNA, 371 bp, Mus musculus
  misc_feature (8)..(364): n = any nucleotide
aagataanggtttttaattgagttatngagatgaagagacagngaagccctgttngctacttacatgaaaagaagattttaaaaaacaatcactgcacaaaatacaaaggggcagggnangcngaggcatngaattcctccccacgntttttctngacttctcaagaacaaattaaagtctccacagcaaattngntctcaaaangccgaanggngaaacagttacnggcttcccgcttcngaatacctctaatngttncccggcgctgcagccngtaggnctccttgncgtgacacagtcgnnagatgaagaagcccaggtngtccacgttctcgangcngacgccgatcaccatgtgctcanggatacg

SEQ ID NO: 66 - DNA, 790 bp, Mus musculus
  misc_feature (101)..(788): n = any nucleotide
aaagagcggctgctgtcggaagcaccgggcgagctatctgttacagtccggcccggggatggctcgggacgcggagctggcgcgcagtagcgggtggccgnggcggnggctgccggcgctgctgctgctgcagctgctgcggtggaggtgcgccctgtgcgcgctccccttcaccagcagtcggcacccaggctttgcggacctgctgtcggagcagcagctgttggaggtgcaggacttgaccctgtctttgctgcagggcggaggtctagggccgctgtcactgctacctccggacctgccggatctggagcctgagtgccgggagctgctgatggacttcgccaatagcagcgccgagctgaccgcctgtatggngcgcagcgctcggcccgtgcgcctctgccagacctgctacccgctcttccaacaggtcgcaatcaagatggacaacatcagccgaaacatcgggaatacctccgagggcccgcgctgaggcggaagtctcctgacggcagacagaatgcagatagttctcatggnctctgagtttttcaacagcacgtggcaggaggcgaactgcgcaaattgcctaacaaacaatggtgaggatttgtcaaacaacacagaggacttcctcagtctgtttaacaagactttggcctgctttgagcataacctgcaggggcacacatacagnctccttccaccaaaaaattactccgaagtgtgcagaaacttgtaaagaggcatataaaaacctgagcctnctgtacagtcaaatgnanac

SEQ ID NO: 67 - DNA, 581 bp, Mus musculus
  misc_feature (21)..(577): n = any nucleotide
ccttgttatttcctttattgnaaagcataaggaaaaaacaggttttcttgngcacacacataaccctatgngcctaaggattcagaantatgtncatttttttaatatgaccacaagatgaaattntttggcacattttcaaatatatttctaatgcaacctntagagagccagaccctgatcaggaacagaganggctggcttgttaaagggctctccagcttnttagccaaaagcagnggtttgtncacacagtactgaaaggnacccgaggagtcgctactcacagtttaaatatgtcacttcactcantttgcncatgtaaataagntttacatgtactgatgaagatggnttccaatgaccctnaaccatgngcttcaaatcaagacaggaacaatgacagcncaatgaaccccggcacatntaggggatcacagcgncgnctgattgtcacatacccggggtgacacactctgggactaagactagcctgctntcacactctgcanatgtggnaaacatacaaaaaatacccaaacactcctgccttcctgtagggcaaanacaggttttnaagg

SEQ ID NO: 68 - DNA, 414 bp, Mus musculus
  misc_feature (29)..(408): n = any nucleotide
aaaggcgcggagcctgctgctgccatggnggctggnggctgggtacactatgtctagactgggggccctgggcggctcccgcgccgggttgggactgttacttggtactgccgccggccttggattcctgngcgtcctttacagccagcgatggaaacggacccagcgccatggccggagtcacagtctgcccaactccctggactatgcgcaggcttcagagcgnggacgccaggtgacacagtttcgggctatcccaggagaagctggagatgctgccatactgcccagcctctcacaggaagggcaggagaaggngctggaccgcctggactttgagctgaccagtcttatggcgctgcggcgcgaggnggaggagcttcagagaagcctgcaaggactagctgncgagat

SEQ ID NO: 69 - DNA, 772 bp, Mus musculus
  misc_feature (134)..(762)
  misc_feature (134)..(134): n is a, c, g, or t
gacggactggacgccgcctccacatccaggtccagagagtccttcctccccatcctcacggtctcgcagctcacttcgtcctcagcatctccactctccttgtcggagtcccgctcgtaatcagactccgcgtnggctgnngtatagcctccctcgctctcggcgtctgngagngcggcccctgaggaggcggngaagtagacagagctggagcccgnggagtcactcctctctctggcaaaagggaacctgcgccgccgggccactctctggttctcttctatatgagagcggacctccccgacaatctccccagctagtccttgcaggcttctctgaagctcctccacctcgcgccgnagcgccataagactggtcagcacaaagtccaggcggtccagcaccttctcctgcccttcctgngagaggctgggcagtatggcagcatctccagcttcacctgggatagcccgaaactgtgtcacctggcgnccacgctctgaagcctgcgcatagtccagggagntgggcagactgtgactccggccatggcgctgggnccgattccatcgctggctgtaaaggacgcacaggaatccaaggccggnggcagtaccaagtaacagtccnaacccggcgcgggagccgcccagggcccccagtctagacatagtgtacccagccaccagccaccatggcagcagcaggcttcgcgcctttccagcacactggcggccggtactagtgganccgagcttnggnaccaagcttg

SEQ ID NO: 70 - DNA, 421 bp, Mus musculus
  misc_feature (12)..(404): n = any nucleotide
aaaggcttgccntncaggccatgcggctggaggtntgcatcgaggcgtttcccatgtttctgctgaaccttctaggcatgnggagctgggtatgcaaaaagngctttccctacttcctgaagcggttcgccatgatatacaatnggaagatggcgagcctaaagcgggagctcttcagcaatctgcaggagttcgccggcccctnggggaagctaactctgctggaggngggctgcggcaccggggccaacttcaagttctatccccccgggagcagggtcactngtatcgaccctaancccaactttgagaagntcttgttcaagagcgtcgcanagaaccggcagctgcagtncgagcgcttngaggnggcagccgnngaggacatgcaccaggtgaccganggcttctgaggaccgtg

SEQ ID NO: 71 - DNA, 571 bp, Mus musculus
  misc_feature (27)..(562): n = any nucleotide
ggcaaatatagaaaccatttatcaaangaatataaangtattgatcaacatttaaaatataacttctgcaaaatcatnttgaaaaatatacatttgtttagatccatacatacaaatgcagctgaaacccttgggcccacccagacttgctctctgtatgaacacaangatatccanggttttgtttcaggaccagnggaatttttcttcttcttcaatacagggttnattngngnagccctggcggncctggaactccatctgtagatcaggctgtactnacagagatccacctacctctgccacctgagagctgggattgaggctntgccaccaaccactcaggaccagngatatttgaccagaagaatccctccccccccgnaccccgagtccttggnaactactctcgggcattacttttagggncccctacatactgnacccatttccctaactatagnggccttctactgcctacggnaatnatattcagcaaagtntgtgctagataagatggnaaattaaacagagaattcccatctgnnctgngcngnnctgngatgacgct

SEQ ID NO: 72 - DNA, 506 bp, Mus musculus
  misc_feature (49)..(502): n = any nucleotide
aaagaaggaactaaacatgggccagcgatgctctgacaccagaggaatngctttcgaagacgtcagagngcctaaggaaaatgtgttaatcggngaaggagcanttttcaagatcgcaangggtgctttngatagaaccagacctacagtcgcagctggcgctgncgggctagcccagagagctctggacgaagccacgaagtatgccctggataggaagacatttggaaagctgctagnggagcaccaaggagtttcatttctgctcgcagaaatggcnatgaaggtngaactcgctaggctcagttaccagagagcagcctgggaggnngactccggtcgccggaacacttactatgcctcgattgcaaaggcctttgctggagacattgccaatcagctagccactnacgccgngcagattttcggnggctanggattnaacacagagtaccctgtggagaagctnangnngnccgccangatctatcanatctatnanggtc

SEQ ID NO: 73 - DNA, 333 bp, Mus musculus
  misc_feature (26)..(330): n = any nucleotide
aaagcgtcgccatccgccaccatggngaacttnanagtagatcagatccgngccatcatggacaagaaagccaacatcgataagggngaggngtctgcccgccaggagctcaaggcacgtgcccgctacctggccgaaaagtntgagngggacgttgctgaagcccgnaagatcnggngcttaggccctgntggcactggccccaacattctnaccgacatnaccaagggngtgcagtaccngaatgngatcaaggacagcgnggaggcnggcttccagnggnctnctnaggngggcgctntctnngagnanancatgcnncattntgcnccc

SEQ ID NO: 74 - DNA, 596 bp, Mus musculus
  misc_feature (78)..(595): n = any nucleotide
aaaggccagctcctgctctgctcctctcccgcctgccgccgcgctgcacgcctcgagcactccctcggccccggcggngaccggggaccccgcagctaccgccatgctgccagngctctacaccggcctggcggggctgctgctgctgcctctgctgctcacctgctgctgcccctacctcctccaagatgngcggnacttcctgnggctggccaacatggcccggcgggngcgcagctaccggcagcggcgacccgngcgtaccatcctgcgggccttcctggaacaagcgcgcaagaccccacacaagcccttcctgctgttccgagacgagacgctcacctacgcccaggaggaccggcgnagcaancaagnggcgcgggcgctgcacgatcaactgggcctacnanagggggattgcgtagccctcttnatgggcaatgagccggcctacgngnggatctggctgcnactgctnaaactgggcngcccatgncgagccttanctacaacattcgtncnaagtctctgctgtactgctttcaatgctncggngccnaangngcagnttncctcccagnnntacatgaagctnc

SEQ ID NO: 75 - DNA, 728 bp, Mus musculus
  misc_feature (203)..(710): n = any nucleotide
aaagcctccacttgcctacttggggcgcgaggaggttggagagtttttttctgggacccaagcaaaggcatccacgctgctgctaagctgaaattgaagctcacacatcctggaaaatgctagcacccataccagaacccaagcctggagacctgattgagattttccgccctatgtacagacactgggccatctatgttggngatggatacgtgatccacctggctcctccaagtgaaatcgcaggagctggggcagccagcatcatgtctgctttgactgacaaggccatagtgaagaaagaactgctgtgccatgtggccgggaaggacaagtaccaggtcaataacaaacatgacgaggagtacaccccactgcctctgagcaagatcatccagcgggctgagagactggnggggcaggaggtgctctacaggctgaccagcgagaactgtgagcactttgtgaatgaactacgctatggagttcctcggagtgatcaggtcagagatgcggncaaggcggtaggcatcgctggagtgggcttggcggccttgggcctcgttggagtcatgctctccagaaacaagaaacagaagcaatgagctgaatgactgcccagtttttgggctcttcttttgctagagggtttggagtttgatttatagattctattgctttataattagggttattttcacaacatacantaaaccacaagaaaggaa

SEQ ID NO: 76 - DNA, 214 bp, Mus musculus
  misc_feature (3)..(192): n = any nucleotide
ctngaggntaatagtatgnngtgaaaatananntaattataaagcantagaanttataaatcaaactccaaancctntagcaaaagaagagcccaaaaacngngnagncattcagctnattgnttctgtctnanngnntcnggagagcangacttcantgngnnccaaggangncaagcncacacnagngnngactactcgactagcccgcatt

SEQ ID NO: 77 - DNA, 629 bp, Mus musculus
  misc_feature (24)..(623): n = any nucleotide
ggggcacagtctattttatataangagatcagttgtctggaaagggatatggtgtccagagtgagataggggaccctgggctngggacccattgttcagataggaaaactgagatcaggccaggtatgattngggttagggagncngngnggagcggggggtnaacctcagcagggcaggcactgngaccctgatcttctcaganttcaggaaagccacgtgcttctcataataggcagcctcattaatgaacacagggtacacaatggagtccccctcatacagtacgtcttctccgctgaaaagcttgaagatgggttcancaggnctnagnggctcaaagtcatggncctgcagntgagggngcacagagncagccaggtcaccatccncagtgcgtgggaagtccacactgcccaagttcctgtanatatccatctcaaaggcgggtaagtccatgccttggttgaagagctcgatgaagtccnnaatggatgccaccagagctngcatccgnnagaacaggncggcccgcagcacgcccctgaggctgcgggcccatctccngacagattccattcttggnnatagattccccctttccagcacactgncggccngnnactagt

SEQ ID NO: 78 - DNA, 200 bp, Mus musculus
  misc_feature (11)..(196): n = any nucleotide
aaagctgcatngnggcgttacccatgtttcngctnaaccttctaggcatgnggagctgggtatgcaaaaagngctttccctacttccngaagcggntcgccangatatacaatnggaagatggcgagcctaaagcggnagctcttcagcaatctgcaggagnncgccggnccctcggggaagctaantcagctgnnggag

SEQ ID NO: 79 - DNA, 278 bp, Mus musculus
  misc_feature (1)..(274): n = any nucleotide
nggcaaatatagaaacnatttatcaaatgaatataaangtattgatcaacatttaaaatataanttctgcaaaatcatcttgaaaaatatacattngtttagatccatacatacaaatgcagctgaaacccttgggcccacccagacttgctctctgtatgaanacaatgatatccatggnttngtttcaggaccagnggaattttncttcttcttcantacagggtttatttgtgtagccctggnggccctgnaacncnatttgtagatcagnctgt

SEQ ID NO: 80 - DNA, 805 bp, Mus musculus
  misc_feature (710)..(750): n = any nucleotide
aaaggtggagctgggtggtgtttggtgcggtacggcggccactcagttgcagcagagcaggtgccatcctgtggaagaaccatgaagcactacgaggtggagattcgggatgcaaagacgagggagaagctgtgcttcctggacaaggtagagcctcaggccaccatttctgaaatcaagacccttttcaccaagacacacccgcagtggtatcctgcccgccagtccctccgcctggaccccaaggggaagtccctgaaagatgaagatgtcttacagaagcttcctgtgggcaccacagccacactctacttccgggacctcggggcccagatcagctgggtgacggtcttcctgacggagtatgccgggccccttttcatctacctgctcttctacttccgggtacccttcatttatggccgcaaatacgactttacgtccagtcggcatacggtggtgcacctcgcctgcatgtgccactcgttccactacatcaagcgcctgctggagactctcttcgtgcaccgattctctcacggaaccatgcctttgcgaaacatcttcaaaaactgcacctactattggggctttgctgcatggatggcttattacatcaaccaccctctctacacaccccctacctatggagttcagcaggttaagctggcactggccgtttttgtgatctgccagcttgggaacttctncatccacatggctcttcgggaccttcggcctgctgggtcnaaaaccaggaagatccataccccaccaagaaccccttcactggctggtcctgtgg

SEQ ID NO: 81 - DNA, 489 bp, Mus musculus
  misc_feature (52)..(481): n = any nucleotide
gggtaagcaggttttattgttgctgctggagagccatggccagccacacatnaggacaggggcagngaggggtggagagtattactgggcagagccgtgaggagctgctcagagcaggaagggaataatgggcatgcgcaggggcgggtagtcgcggaactccttcaggtagctgcggngtttgcccttggcccagatagtcatctgggtgaagcccaccagggagaagagggccactgggacacactgagtcaagatggcaaagccaatccaggagcccacctnataagtgtagttgggacaggacaccaacaggaacagccaggtgaaggggttcttggnggggtatgggatcttcctggttttcgacccagcaggccgaaggtcccgaagagccatgtggatggagaagttcccaagctggcagatcacaaaaacggccagngccagcttaacctgctgaactccataggtagggggngtgtagag

SEQ ID NO: 82 - DNA, 465 bp, Mus musculus
  misc_feature (59)..(460): n = any nucleotide
aaagcgctggagctacagccgttactgccgccgccgccgccgccgccgccgaggcgttngatcgttggcaatgtcaggctttgataacttaaacagcggtttctaccagacgagttacagcatcgacgagcaatctnagcagtcctatgactatggaggaagnggaggaccctacagcaagnagtatgctggctgagactactcgcagcaaggccgattngtccctccagacatgatgcagccacagnagacatacactgggcagatttaccagccaactcaggcctatcctccaacaacanctnagccattctatggagacagcttngaggaggagccccctctgttagaagagttgggtatcaattttgaccacattnggcaaaaaacactaacggagctacacccccctgagggcagntgacggcagcatcatgaatgatnacggacttgngcaggncccag

SEQ ID NO: 83 - DNA, 505 bp, Mus musculus
  misc_feature (31)..(497): n = any nucleotide
aaatggccctatgatgcagaactctcctttntcctgcgcacgggtacacggtacggagnggacactnacctgttcagngnggagtcgccgcaagagctggcagcctggacccgacagtnggaggatggctgncatcgggctgctgaaggcgtacaagaagngtctacagcctgcacgnggaacggccgnccctgcagcctgtctgngcacatcgacaagggcttcaccctgngggcagctgagcctggagcagcccgagccatgctgctccgacagcccttcgagaaacttcagatgtcatcagatgatggcacgagtctccttttcctggacttngggggngctgaaggagagatccagctggacctgcactcgagtcccaaaacgatggtcctnnatnatccactctttcctgtccncnaaggncacccnctngncgcncanggcctagangcctgncccgntgnaccagnnccttgaaagcaggcaatctgatncatcctcg

SEQ ID NO: 84 - DNA, 375 bp, Mus musculus
  misc_feature (2)..(364): n = any nucleotide
gngctnanangntgntnattattnaanangaangangggananaggacnagganaaaacccaaatgccccacggngnttaaggggaagngaacnnaaaggntnctcttcttnctcttgccactgacccanaanantccanntgnananatctnanggnggnnaangggcctcnnnnaggcctggcanntntgctgnnggntggggacgggatccncanagggnnangncccangctngnaaaggtttctgngtacncnaaaaatntnttttaaaaaggcncnggnggangnnancnntangaangctnnccaacncaanccnnacttgctnncccaggnctgagntntgnttcctnaggctgnnctctgaggccc

SEQ ID NO: 85 - DNA, 758 bp, Mus musculus
  misc_feature (33)..(737): n = any nucleotide
aggctgtgcgaagcgggcacgcgcggacccttnaccggccgcggagccgctatgggcccgccaggcccctggcgtggcgcggcgcaaggaccggctccnagccaggattgaaattccaagatggatgatcaggaccctgggggcattagcccccttcagcaaatggtggcctcaggagccggggctgnggacacctccctcttcatgacacccctgnatgnggtgaaggtccgccttcagtctcagagaccctcggcaaccagcgaattgacaactccctccagattctggagtctctcctacaccaaatcatcctccgctctacagtccccagggaagtgcctcctatactgnaatggagtcctggagcccctgtacctgtgcccaaatggtacccgntgtgccacctgntttcaggaccccacacggttcactggcaccttggatgccttngngaagattgngcggcatgagggcactaggaccctgtnnannggcctccnagccaccctggngatgaccgngccagctactgctatctacttcactgcttacgaccaactcaaggccttcctgtgtggcagtccttgacctctgacctctacgcacccatggtggctgntgccctcgcccgaatgggcaccgtgacagttgcagccccttgnagctcgtgcggaccaagctgcaggctcagcatgtgtcataccgtgagctggcttnctctgttaagctgcngngactcagggtgctggcgctct

SEQ ID NO: 86 - DNA, 367 bp, Mus musculus
  misc_feature (1)..(355): n = any nucleotide
nnncntttnncncacagaagaccatcatntttagacaaggaatgaatggngccaagatactgcctggcccaagntcctcggnccaagctataacaaacatgnggatncaaaaaagggcgatcatcagatttgntcttgaattcggnaggcggnagaggggcaatcctggnaagggaaacagcaggaatccaggtctgaggacagcctgggagacnggactggnaggnaaggcacttgnctcagtctcctgatcccacccgtgcaagagccgnnngctccccgactttcagcggcccagaggctgctnctggtngagcctctgnaagaagcttttgccaaactcnnaagtgctgancatgatggccag

SEQ ID NO: 87 - DNA, 557 bp, Mus musculus
  misc_feature (31)..(553): n = any nucleotide
aaagcttcacttcgccctccagccgcggagnctgcagcgcaacttccagatagcggagnggcctcagctgcgagccgagcggaggcggcagcgttnctcaggacacccgcagatcaccttttccccgcgacttcgccatggctgagngctgagtaccggtatgcccacggccgatgngtatccctccaccctatgctgacctcggcaaagctgccagagacattttcaacaaaggattnggcttagggctgnngaagctggatgngaagacgaagtcatgcagcggngtggaattttcaacatctggctcatctaatacagacactggtaaagttagcgggacctnggagaccaagtacaaatggngagagtatggtctgactttcacagagaagtggaacaccganaacactctggggacagagattgcantttgaagaccagatttgtcaaggatagaaacttgactttngacacccaccttttcaccgaacacaggnnnaaggananngtggtnaataatcaagtcctgctttaccaagnagggngnagntgtc

SEQ ID NO: 88 - DNA, 636 bp, Mus musculus
  misc_feature (83)..(631): n = any nucleotide
cggttttcataaacgtctatttcatcattggtgggtagcacatttaacagttaaatacatttaaataatgtataggaggccgnaccacggcagcactgataaccatccaactaggaaccagccaacagtgactgtctaaatatttaaaatacagctctngcttcatcatcctttgatgtgatcaccttctgggggaaggaaggggagcctgctggtcgcatggaaatatattaaggccaaatcttctgatattcgttcccagaggtttcttttaactggattaagcctccaattccaaggcaagcccaagtttgnggcctccagcattaaagctcttcccgtctaccagagcagacagtgtaagcttcacaccaggcctcagagtctgagtatagcccactccaattaaactagagttgttgacctttgcagagatagaagcagtaggatccaactggtatttagctgcaatgccaaaacgagtgcagcnggnacctgatgtccaagcgaggnttactgangtgtcaaaatcttnacatactttctgatnaatttgatcctccaaattctgtcccattatttacatttgtgtgnagctggaagtccccagtcctgtagccgacngcanagntactc

SEQ ID NO: 89 - DNA, 808 bp, Mus musculus
  misc_feature (34)..(799): n = any nucleotide
aaagggaggaggaagcccggagcggagcggggcntctggggggggtggacccgccgcggctgctgctgccaccgccgccgccgccaccaccgctcgtggggctcgtggcgtgaggaaggaggacgagtgagaccccggggcgagcgggcggcggcgccgctgctgctgctgctgctgcgggagggtcggcggcgggacggcgatggcggatatcgacaaactcaacatcgacagcatcatccaacggctgctggaagtgagagggtccaagccaggcaagaatgtccagctccaggagaacgagatccgaggactctgcctgaagtctcgggagatcttcctcagtcagcctatccttttagaacttgaagcaccactcaagatatgtggngacatccacgggcagtactatgatttgctccgtctgtttgaatacggnggctttcctccagagagcaactatttgtttctcggggactatgtggacaggggcaagcagtccctggagacaatctgcctcttgctggcctacaaaatcaagtatccggagaacttctttcttctcagagggaaccacgagtgcgccagcatcaataggatctacggattttatgatgagtgtaaaagaagatacaacattaagctgtggaaaacgttcacagactgttttaactgcttgccgatagcagccatcgtgnacnagaagatattctgctgcatggagggttatcaccagatcttcaatctatggagcagaatcggccgaaatatgagaccaactgatntaccagatcaaggncttcttttg

SEQ ID NO: 90 - DNA, 680 bp, Mus musculus
  misc_feature (96)..(679): n = any nucleotide
aagttaacaagcttgcatttaataagtctgaaaccattctcagcacatggcattgtacacgggcatctgtgcaaacagattcatttaacaggtcgnagtttaaaaaagtcatagatactgngagttctgtataaaccggnggacggnaagttagttcctttngatttataagcctcaatgtcaccgnagaataaagaatgtagccaaagaaagcattatcggtcactcgtataggacagagttgtttctataatttgaagctttctgaatggacggnttcaggcctgatccaactgtaaaaagatcactcagtgaatagactatatgggaactgtacaaagtgtcattaacttncatcattaatagcttactcagcactataccactattgctagttaaaataacctgcttctgaggccccacggagggaggcggcctgtgcacgcagcctcgatgccctggccacctcatccccagggcgtgccatacagtccaacagaaactttggctttaggaaggaatcacagacnttgaaaagaatggctttaatcattattaaatgtgcagngggaaggagtgtgcttcagatagtctgggcagggctggcggcaggcaggtcactcctgctgcacagctgcagacactagtttgtcatgacaagacaatgagggaaagcagnc

SEQ ID NO: 91 - DNA, 785 bp, Mus musculus
  misc_feature (148)..(760): n = any nucleotide
aaagaagaagagggggctaagctgagtatagaggtgctccagaccagcctgcagaaggaactgactctaaacaaaggccaggcctccgccatggagctgctgcgctgccccacgcttcggcgcctcttcctctgcctctctatgctgnggtttgccactagctttgcctactacgggctggtcatggacctgcagggctttggggtcagcatgtaccttatccaggtgattttcggcgctgnggacctgcctgccaagtttgngtgcttcctagtcatcaattccatgggccgccggcctgcacagttggcctccctgctgctggcaggcatctgtatcctagtgaatggcataataccgaggggccatacaatcattcgcacatccctggctgtactagggaaaggctgtctggcttcctctttcaactgcatcttcctgtacaccggagagctgtaccccacaatgattcggcagacgggcctgggcatgggcagcaccatggcccgggtgggcagcatagtgagcccactgataagcatgactgccgagttctacccctccatacctctcttcatcttcggcgctgnccccgtggccgccagcgctgtcactgccctgctgccagagaccttgggccagccgctgcctgatacagtgcaggacctgaagagcaggagcagaggaaagcagaagcaacagcagctggaacagcanaagcagatgataccactccaggtctcaacacaagagaagaacngactctgaaaatggagaggcgtcac

SEQ ID NO: 92 - DNA, 620 bp, Mus musculus
  misc_feature (55)..(619): n = any nucleotide
caaggtagaagaaatttatttaattgtctgggattctttgcaatgtcctggaggnggaagggacaggagctggaggagtgaccactgagctggaagatggctgaggaagagctcattctgcttaagaagctgcacacagttagagctttngttcctagtaggtctattgaagtgacctttggggaggcatttctctgaatggcaggctccgcatttagatggcccagtccctccactcactccccctcctcatagatggngggacctgcagaaccccactccctttagngctgagngacgccctctccattttcagagtccgttcttctcttgagttgagacctggagnggnatcatctgcttctgctgntccagctgctgangcttctgctttcctctgctcctgctcttcaggtcctgcactgtatcaggcagcggctggcccaaggtctctggcagcagggcagtgacagcgctggcggccacggggacagcgccgaagatgaagagaggtatggaggggtangaactcggcagtcatgcttatcagtgggctcactatgctgcccacccgggccatggngctgcccatgcccaggcccgnctgccgaatcattgng

SEQ ID NO: 93 - DNA, 491 bp, Mus musculus
  misc_feature (8)..(487): n = any nucleotide
aaagacgntaccctggagttcaccagcatcgacgctcacaaaggcgtggccccatcaagacgnggngattnggaaatactnggttatngcatgatccagnggctcagcggctgtcttcctngggaagataacttgaaagatcctaactacgttagggattccaaaattagatacagagacaactgtcgcagctttganggagaaatgctttcctgagaaaaataagccaggtgagatcgctaagtacatggagtctgngaaactactggaatacaccgaaaaacctctctatcaaaacctacgtgatatccttttacaaggactaaaagctntaggaagtnaagactnacggcaaactggnttttagngctgagganaacggaagtgngaancnaagaccagcctcaaagnngctttaanaaanaancanaanaaagcncngncgagcgcntgtncnagtgacatntgagngctctantccacncancgtt

SEQ ID NO: 94 - DNA, 628 bp, Mus musculus
  misc_feature (40)..(623): n = any nucleotide
atgaacctgagatttattttttcttgtcaaagtaacgagnctcttatatggaaagcggctgtatatctctgaaggagcagttagagagctgcttctgaattcacccagaattgctactgaccctgagcccacatttcctttcagctctccagtctcgggttnggccagaggagagtccactcatgtgaaacattcactccctgacattcccattctagggtctnaaagaacggnggaggagctggaagcaagggcaagaggagactccagtaggttngggcttctactataaattctcttctgagagcacagnagaggggnctgcgtgtctgcgncacggngaggctcacgctttatctgctggaacttgcactgngtccagccataaaagccacagtngagcagcccctgggatgctgctgagagagcctggagcacagagagggccatgtgcaggaaggnctcctgaggctctgtcagcttcatgatcagcagggagacagctgnaccccagcagcacacaaaggccactgnnaagaaacgcactcgctggcccacagtgtgaatcactgcccactgctnactctccagaaggcccgncgactttacanacttcttatatnacatc

SEQ ID NO: 95 - DNA, 622 bp, Mus musculus
  misc_feature (30)..(616): n = any nucleotide
aaagctgcggcaggcattctcggaggaaannagccaaggactaactacgancatgagatnggcagtgattngctttngcctatttggcattgcctcctccctcccggngaaagtgactgattctggcagctcagaggagaagaagctttacagcctgcacccagatcctatagccacatggctggngcctgacccatctcagaagcagaatctccttgcgccacagaatgctgngtcctctgaagaaaaggatgactttaagcaagaaactcttccaagcaattcnaatgaaagccatgaccacatggacgacgatgatgacgatgatgatgacgatggagaccatgcagagaacgaggattctgnggactcggatgaatctgacgaatctcaccattccgatgagtctgntgagaccttcactgctagtacacaagcagacactttcactccaatcgtcccctncagtncgatgtccccccgacggcctgaggntgatagcttnggcttatgggnctganngtcccaagntctagnnagttttcccaggntttcctgnatganaccangcnatcccntggaatncccaccntattgnaggnnaacccttaacccnttcttt

SEQ ID NO: 96 - DNA, 653 bp, Mus musculus
  misc_feature (158)..(640): n = any nucleotide
acatgtacatgattttggaataatttaatactttaacctcaagatacaactatattctaagaccattattttaaaggaacggatccttacaagaccaaaataacccatatagcacgaggttggtttagcctttcttcttctttcaacaaacgtgcacnacatgtttcagtagcaaggccgatgccatggatatgagagctgngatttgcagggaccaaccacatntagaaccggggaggccaatcagacggngggtngggngccattctacgtaaatcagcaggtgacatnacaacacgctgggtgcagcctcgcaactgtccattaggtttcctttttcttgatgatcagaggncctacgttgncgccagtctcttcgttgngctttatgtgagccccatcgcagaaggggaactttttagacctccagcatnggcagtacacggccttatcccccagatcctccatgtcgaaggcatgcaccaccttcggnttgactttctggatctgaagattcaccatagctttggagcgattctctttagcgtagaacttcttgnaagccaggnaaccgagagcggctgtgccagcagcaaaggtgacgggccgcgaccactcaacttncacagcggagnttgnngctgaggcccatg

SEQ ID NO: 97 - DNA, 529 bp, Mus musculus
  misc_feature (34)..(525): n = any nucleotide
aaaggagcaacgcgggtcttcccgctgttgcttntcgcggccacggccgagcatacgtccccggcctgaggtggnggtggcgagcccaccgccgtttgctgcgacctcatggaaggtggcggaggaagtagcaacaaatccaccagcgggttagctggcttcttcggagccggaggagcgggttactcgaacgctgatttggccggcgtcccgctgactggtatgaaccccctgtctccttatttaaatgtggatccacgctatctcgttcaggatactgatgaatttattttgccaactggagctaataaaacccgaggcagatttgaactagctttctttaccattggaggatgttgcatgacaggggccgcattcggggcaatgaacggtcttcgtttaggattgaaggaaacccagagcatggcctggtccaaaccaagaaatgtacagattttgaatatggtgactaggcaaggagcactttgggctantactctaggctccctggctttgctctatagngctt

SEQ ID NO: 98 - DNA, 116 bp, Mus musculus
  misc_feature (94)..(105): n = any nucleotide
ctgttttcaccctttatttggaaaacagggcaacatttaagttttagattttttaaatgaattaacatggtaataaaaagtatggcctgaattnggagagtaagnggttttccagt

SEQ ID NO: 99 - DNA, 717 bp, Mus musculus
  misc_feature (17)..(716): n = any nucleotide
aaagggcctccgcactnccaagtcatttgncggctacnngnctatnagngcanagcagggtgtcanggaccnagttgctnnaggcngnnaagagccngctgcaaggtnaatcctacagtacgtgnaaactctgatggaagngatncccaagatctgncgncttccgcnacatgagtacggctcacctggcatcctggagatcttccaccnccagctgaaggacattgaggagaatgcacnagctgaaaaccgtatgcttccagaacctgcggcaggtgggaaatgctgccctcttcngccngcttatangagcanagnctgnctttagaanaantctgtgaccngttgcatgcagctcntttccagaatntcttacctnngaatccntgnanaagagggnnagagagtntgaatnccnaaatnaacagactncaatccaantattgccccantgtaccctngccccaatgnttgaaanggctgnggaccccnagcaaancgcaactgcncganacggngnctngctgnccaangnancgcctcatgnngcggcatgnccatgtttgaagttcncctnacncncnccncancttctggcntgatccccnatcnggcgcngnccccctcaccnagcnnttggtgnnatncnngcncnanagangccttgngcttcncancctctagctaacccccngcccctnngtatnncttcactnc

SEQ ID NO: 100 - DNA, 662 bp, Mus musculus
  misc_feature (37)..(658): n = any nucleotide
aatacaattagttggtattatgctcgtacaggatgancgaccccantntcccntcntanctgctgtaatattcggcatgaaaatacttgttaataccgtaanggcaacaactagtaaccgtattctcagacttcccaattccaaaggcatatacaattttagtatagaaaaataagtaaattttataaagttaagctttcagatcaaaagtaggttcagacataanggaaaatagcccctaaaaatttcaatatagtttacataaagacaaacatgccatcagttactgggatgctgngagcttagccctcagctacnggctagggactggaggangggnggctggaagcagcgtacatgctccacaggngngctctcgccatcaccagatttcaagtacttgtccaggatagtgatgatttcatcgttgagaatctggaacttgcggatcctctccaccatcttcttcaatggcacatttttgatgatctcatctttgccatcatgnttctgaactttgagangatgatagcagaaatccaacacagcaaagcgccgctgntgtccaagangtacaatgatcatgcagccagcccagaggangcccatctccaaaancacngctccacttgtaaaactcgnggggccccctaccgnnaaat

SEQ ID NO: 101 - DNA, 385 bp, Mus musculus
  misc_feature (200)..(200): n = any nucleotide
aatctggaacacaaaagtttattagagtaaaaatacatatataatatccaacattaaaattatctcatgtcacatgttttacctgcattagtttttccaaaaaaatgctttaaaactctatgccttaaataattttgcatatatgtaaacaaatcttagattaccgaagatgccattatacctgttagatattgaacatnaacctttaggaatgggaactacaagtttcactctattacactaagcgctactctgaaggaaggagggaaggacggaaggaaggacagaaggaaggataacccacttgagatgaggtaagagtaagagtttagtaccaaatgttgacacaagaatgttaaaggcattcatggaaagtcactccgct

SEQ ID NO: 102 - DNA, 425 bp, Mus musculus
  misc_feature (17)..(413): n = any nucleotide
aaaggctgctgtcaggnggtcccttttatggatgggctcctgnggncgctgcgcagnggntgntcgacttccgnagngncnctcgggccancgagcgccgtctttancnagctgccanggctgtggccgctgtcggccgccngagagccctgcgctgtccgctgnagctcctgctgtnactcctgntgnnagccgccctgcgctgngcngtaacgaccctganagaatactcttgcggtatgtgaaagctcttaccctctactccgaccgctacaccacctccnnggnggctgnaccctatcccacagttgaagcgtgnaggaggcaccgangnntganacgncctataccccagggnnnatacagtgcntnactacaggntgnnnancctacctatgacagtgcgaactntnagactcgcctct

SEQ ID NO: 103 - DNA, 186 bp, Mus musculus
  misc_feature (3)..(183): n = any nucleotide
ctnnatngncttccagnacngnanntnccnagcctcaaaccannaangnaanacnacgntgnanagngangnncagnagacngaagganctnatcannncgannnatgganganntagntancacaananggncngntnacgnnggagctgnaggangacggnancgaggcggacacatnagnagt

SEQ ID NO: 104 - DNA, 501 bp, Mus musculus
  misc_feature (29)..(489): n = any nucleotide
aaagcctgtgtacgccaccatcggctttnggtatcgncaacacggccttcantgnggagtcgctgnttgtngtagagcgagctggacgacggaccctgcacctcattggcctggctggcatggcaggctgagctgngctcatgaccatcgccctggccttgctggaacggctgccttggatgtcctatctgagcatcgnggccatctttggctaagnggccttctttgaagtaggccctggtcctattccatggctcattgtggccgagctgntcagccaggggccccgtcctgctgctatngcngaggctgncttctccaactggacctcaaacttcattgtgggcatgngcttccagtntgnggagcaactgngcggnccctacgtccttcatcatcttcacggagctcctcgngctcttcttcatcttcanctacttcaaagtccctgagaccaaaggcctgaaccttcnatgagancgcttccgncttccggcaggc

SEQ ID NO: 105 - DNA, 410 bp, Mus musculus
  misc_feature (19)..(366): n = any nucleotide
aaagcttacccagcagggngagagatttntatcaacaagagaaggcacggangggctcagcgggactcagagntntngcctnggngncnggtccggaacnggggcagctgaccttccttggccnggacggaatcatngaccctcctcgaacnggngngaaggaagctgncacaanantcatngcctcaggagtctccattaaaatgatcactggagattctcaggagacngcaantgccatcgctagacgncngggattgtactctaagacttnacagtccgagnctggggaagaagtngatacaanggaggngcagcacctttcacagatagagccaaaggnngtagtattttacagagcaagcncaagacacaagatgaaaattattaagtctctacagaagaacggg

SEQ ID NO: 106 - DNA, 605 bp, Mus musculus
  misc_feature (10)..(596): n = any nucleotide
gcctttaaangattgagttnatttgtgngttagtaagaaagccctataaccataaatagtncaatatttaaaagtaaaaaaaatatttatatccatctaagacagacagtgtattngtncattagaattctttaagtgcagaaggtggntcaggnttngcctttngnattttttaattcagtacttgcccatatgttgaagttcactnaattgnaaaagccaattnaaatcaaggaatgtgttattnaaagntgcntaatttcattngggactaccaacaaagttnaaatcaagnctttaacattaanggtcatttttagtnaanggaaagttaaaaagtccttaagtcttcattatcttcttgaacttgcccttctaaatgattctnagactgcantncctagtttgcnaattaaaaaaaaatgcnntatgcatcatacttnaaggaaagacgatgaangctnaaccagcnttcttcctggaccttctcccggctcctttcanccttctttataaccctcagncacantgcacactgacnaggtaaggcccnaaagaancancagatcccngnatnctcaggctctccgnttttnaaaaacctt

SEQ ID NO: 107 - DNA, 573 bp, Mus musculus
  misc_feature (493)..(560): n = any nucleotide
gcttgaccttggaggcctcagcctgagacctcaaagcagcctccagaactccggcagagttcctctgtctcgtcttgccgattgaaggtccccgtttctccaatttctctccatcttctgggaggtagcaggaaatcagaatcatggttggtttcaaggccacagatgtgcccccaacagccactgtgaagttcctgggggctgggacagctgcctgcattgcagatctcatcactttccctctggataccgccaaggtccggctgcagatccaaggggagagtcaagggctagtgcgcaccgcagccagcgcccagtaccgtggcgttctgggtaccatcctaaccatggtgcgcactgagggtccacgcagcctctacaatgggctggacgccggcctgcagcgccagatgagctttgcctccgtccgcattggcctctacgactctgtcaaacagttctacaccaagggctcagagcatgcaggcatcgngagccgcttctggcaggtagcaccacaggtgccttgncgtggctgtngcccnagcctacagatgtnnaaaggcccgtttc

SEQ ID NO: 108 - DNA, 635 bp, Mus musculus
  misc_feature (30)..(511): n = any nucleotide
gccagcctaacacttctatgacaaaccgangaaaattataaaaacttttcaaatatattgctcctcagaccccctatccacaccattaattattttaacagcctgattactgccactaatattaatagctagccaaaaccacctaaaaaaagataataacngtactacaaaaactctacatctcaatactaatcagcttacaaattctcctaatcataaccttttcagcaactgaactaattatattttatattttatttgaagcaaccttaatcccaacacttattattattacccgatgagggaaccaaactgaacgcctaaacgcagggatttatttcctattttataccctaatcggttctattccactgctaattgccctcatcttaatccaaaaccatgtaggaaccctaaacctcataattttatcattcacaacacacaccttagacgcttcatgatctaacaacttactatggttggcatgcataatagcatttcttattanaataccattatatggagttcacctatgactaccaaaagcccatgttgaagctccaattgctgggtcaataattctagcagctattcttctaaaattaggtagttacggaataattcgcatctcc

SEQ ID NO: 109 - DNA, 793 bp, Mus musculus
  misc_feature (402)..(772): n = any nucleotide
ggattggcggggttctctttgaccacataaatcaattctttcttttgggagcctcaatggtggcaactgcagcgggactttatctcatcccgttctgcaagacagcagtcttattgatcatcaccatgtctgtctttggagcttccgttggtgttgtggatacaggtgcaaatgttctcatcttggatctctggggggacaaaggagccccacaaatgcaggccttgcacttcagtttcgccttgggtgcctttctggctcccctgctggctaagttggcctggggtacagcacctgctcagaaccacaccgagtccgaccttgacactctgatgctgaaccgatcctccaacggcacctcagactccgtgtttgcggtacccgatgacatgaatctgctgngggcatatgcttccattggcacctttattttagtagtttctgtctttctgnnnggtctgttttgtaagaaacattcaaggcagaaaaaacccagagcatctgctgagggagctcgaagggctaaatatcacagggccctgctatgcctgctcttcctcttcttcttcttctatgtgggagccgagataacatacggctcttacatattctccttcgccaccacccatgttggcatggaagagagcgaggcagctggcttgaattccatcttctggggaacctttgcagcctgcaggggcctggccatcttctttgcgacattcttacagcctggaaccatgatcgtgctgagcaacattggcagcctggnctcatgtttctttctggtact

SEQ ID NO: 110 - DNA, 724 bp, Mus musculus
  misc_feature (16)..(718): n = any nucleotide
aaatatcacagggccntgntagcctgctnttccctcttcttcttcttctantggaanccgaagntaccatnncgnttcttancagtttctcctttcggccnccnacccagtggcatggaagagagcgaggcagctggcttgaattcccatcttctggggaacctttgcagcctgcaggggcctggccatcttctttgcgacatttcttacagcctgganccatgatcgtgctgagcaacattggcagcctggtctcatgtttctttctggtactttttgacaagagccctctttgtctctggatcgcgacttctgtgtatggagcctcaatggcagccacgtttcccagcggcatctcctggattgagcagtacnccnccttaactgggaaatctgcagcattctttgtaattggctctgccctgggagatatggccattccagcggtgatcggaattcttcagggacnctacccagatctgccagtagttctgtacacatgtctgggctcagccatattcacagctattttatttcctgtgatgtataaattagctaccttgcccctgaagagagaggaccagaaagctttgcccactagttctagactgtgaggaagagactacatgagaacttaaaaaaaaaaaaaaagggcggccgctcgagcatgcatctagagggccctattctatagtgtcacctaaatgctagagcncnnctgtca

SEQ ID NO: 111 - DNA, 1243 bp, Mus musculus
  misc_feature (402)..(1237): n = any nucleotide
ggattggcggggttctctttgaccacataaatcaattctttcttttgggagcctcaatggtggcaactgcagcgggactttatctcatcccgttctgcaagacagcagtcttattgatcatcaccatgtctgtctttggagcttccgttggtgttgtggatacaggtgcaaatgttctcatcttggatctctggggggacaaaggagccccacaaatgcaggccttgcacttcagtttcgccttgggtgcctttctggctcccctgctggctaagttggcctggggtacagcacctgctcagaaccacaccgagtccgaccttgacactctgatgctgaaccgatcctccaacggcacctcagactccgtgtttgcggtacccgatgacatgaatctgctgngggcatatgcttccattggcacctttattttagtagtttctgtctttctgnnnggtctgttttgtaagaaacattcaaggcagaaaaaacccagagcatctgctgagggagctcgaagggctaaatatcacagggccctgctatgcctgctcttcctcttcttcttcttctatgtgggagccgagataacatacggctcttacatattctccttcgccaccacccatgttggcatggaagagagcgaggcagctggcttgaattccatcttctggggaacctttgcagcctgcaggggcctggccatcttctttgcgacattcttacagcctggaaccatgatcgtgctgagcaacattggcagcctggtctcatgtttctttctggtactttttgacaagagccctctttgtctctggatcgcgacttctgtgtatggagcctcaatggcagccacgtttcccagcggcatctcctggattgagcagtacnccnccttaactgggaaatctgcagcattctttgtaattggctctgccctgggagatatggccattccagcggtgatcggaattcttcagggacnctacccagatctgccagtagttctgtacacatgtctgggctcagccatattcacagctattttatttcctgtgatgtataaattagctaccttgcccctgaagagagaggaccagaaagctttgcccactagttctagactgtgaggaagagactacatgagaacttaaaaaaaaaaaaaaagggcggccgctcgagcatgcatctagagggccctattctatagtgtcacctaaatgctagagcncnnctgtca

SEQ ID NO: 112 - DNA, 528 bp, Mus musculus
tgcaatggcggacgtgtctgagaggacgctgcaggtgtccgtgctagtggctttcgcctctggagtggtcctgggctggcaagcggatcggccgtggaggcgttacctagactggaggaagcggaggctgcaggtcgggctggcaacgactcagaaaaagctggacctggcctgagcacgcgctgcagcccgagtccgccgggttctcactccctaagcccaacgcagcccggatcgtgggagccgcgcgacccaggagtcgtccttgcacggcttgcaagaacatggcttgcttcagaaagaaaatagttttgtcttctctaacaacttactttcagcttgtcgaagatgaaaataaaaagcactggagagaaataatttcttgcactttatgaatctatttttaaaataaaaaaattaaacatctttgaatctttttcctcctcacaaaagaaagcagtatttttgcctaccattcagtttgctgcagtaagagatttggagcctgaaagcagagactttctgatg

SEQ ID NO: 113 - DNA, 646 bp, Mus musculus
  misc_feature (27)..(542): n = any nucleotide
tgaggctgcaggccaagctggcaacgnctcagaaaaagctggacctggcctgagcacgcggctgcagcccgagtccgccgggttctcactccctaagcccaacgcagcccggatcgtgggagccgcgcgacccaggagtcgtccttgcacggcttgcaagaacatggcttgcttcagaaagaaaatagttttgtcttctctaacaacttactttcagcttgtcgaagatgaaaataaaaagcnctggagagaaataatttcttgcnctttatgaatctatttttaaaataaaaaaattaaacatctttgaatctttttcctcctcacaaaagaaagcagtatttttgcctaccattcagtttgctgcagtaagagatttggagcctgaaagcagagactttntgatggaatctcnccttggtacagcctggaggcagatntgatcaacggaccattatgagtcatttttctagacatattcagaaaacctaggagctgtgtcaaatgcctgaattaagcattacaaatgcaagatatttgcnctttgaagaatgtagagagtaaaaaaactaaaattaaaaaaaataatgcatgtgatataacggaatatatatgtgaaagagaaaaaaaaaaaaaaaaaaaaaaa

SEQ ID NO: 114 - DNA, 792 bp, Mus musculus
  misc_feature (769)..(784): n = any nucleotide
gatagttttgaacttccgattcccctgcctccccaccccagggctgtgattgcaggtgtgcgcttgggactgaccccaggctttgtcggtgctagggcagcactctgcgactgaattaggtcccagccacttctctgtcttttaaaagaacaaaacattgctaaatgtgccattgttgctttgagttttaattcttttttttttctttctttcataaaacattacagtcttaagatattaaagacttttattctggttctatttctgtcttttcactcaaaactggttttacaaatgatgccttgtttacagaaagctctctaccacagggcctagtcatgtgtaaagtctcagtttctctctggagtatcttggagcctagcacactggctttaaaggacacagctaagaagctgatatcttgacagtgtttgtagacctttgttataaaaatgaatgtcctggaaagggttgggagggagttcaacaacaaagaaacaagaatgtcatgtttaaatttaatagttgtctaaaatgtcatctcaagtcaagtcactggtctgtttgcatttgataggtttttatactaactagcattataagattatttcataattagaaaatacctgtggatatttgtataaaagtgtgaaataaattttttacaaaagtgctcatcgcttgttaacacagcatcatgtatgtgaaagcaaactctaagattataaatgacaacctgagttgcctttctttgtatttcatcaagccnaagtaaagctttcantatttaaa

SEQ ID NO: 115 - DNA, 837 bp, Mus musculus
  misc_feature (5)..(825): n = any nucleotide
gcaanggcggacgtgtctgagaggacgctgcaggagtccgtgctagnggctttcgcctctggagtggtcctgggctggcaagcgaatcggctgcggaggcgttacctagactggaggaagcggaggctgcaggacaagctggtaacnactcagaaaaagctggacctggccngagcacgcgctgcagcccgagtccgncggnntctcactccctaagcccaacgcagcccggatcgtgggagccgcgcgacccangagtcgnccttgcacggcttgcaagaacatggctngcttcagaaagaaantagttttgtcttctctaacanctaactttcnncttgtccaanatgaanatacgnagcnctgganaganntaatttcttgcnctttannaatctatnnttaaantacananattnancatcttngantctttntnctnctcacaaaaganagcagcatttttgcctaccattcagttngctgcantaagagatntggagccngaaagcagagactttctgatggaatctcaccttggacagcctggaggcagatctgatcaacggaccattntgagtcattattctagacntattcagaaaacctaggagctgngtnaaatgcctganttaagccttacnaatgcnagatntttgcactntgaanaatgtanngngtaaanaaactactattagtccaaataatgcatgngatntaacggaatatatatgtgaaagagaaaaaaannnannnaaaaaaaaagggcggcccgctcgagcatgcatctagagggcctattctatagtggcncctcaatgctanagcttcgctgac

SEQ ID NO: 116 - DNA, 252 bp, Mus musculus
  misc_feature (45)..(227): n = any nucleotide
agggcggccgctcgagcatgcatctagagggccctattctatagnggcacctaaatgcttagagctcgcttgatcaagcctcgactgngccttttaagtttgccagccatctggtgnttncccctcccccgggcctttccttgacccttggaaggngccacttcccaattgnncctttcctaaaaaaaatgngggaaaatggcaatcgcaattgncttgggtaaggnggcaattctaattcttgggggggat

SEQ ID NO: 117 - DNA, 869 bp, Mus musculus
  misc_feature (72)..(863): n = any nucleotide
gcatccctccatggcctctgcattggcttctgctttctgacctgcttgagttccagtcctgacttccttggngatgaacagcagtatggaagtgtaagccgaataaaccctttcctctccaacttgtttcttggtcatgatgnntgtgcaggaatagaaaccctgactaagacaaattggtaccagcagagtggggtattcctgtgacaacctgaccatgttttggggaggactgtggatggactttggaactttgggcttaaagatccatccgntgntaagagctctgtcagatgttgtgtaggagcttggaagataatgttgagaacactgnagaanatggaggnctggtntgngaaatttnagagggaaaantaaagactctttcagggccattgctgttttgaatgtgaagattctgtagnnctggatagctggggctnaanaatcanctgtgnttaacangatccagaactaccaangcanaaactttgcattactgggactattgatgcntnnntagctgnagctaacaaattancggtgattaanaaagagaccagcntcattnagggtgacatctttctgggnangtgtttcntgaaagcacanngatncntgtgnttncagagatggccaaangctgtaccncctgcttgcaannanggacttgggnataantgtaanngggtcnacccncntngntactggggttttttaanggccntttaacngggntcnacggccacagncanccttcagggcctnnggcancctggttaaaaangccccattnngaaanggnccaaattgnctttnaaagggtacnaggcctncaaacttgcccaaattgtanagggccncccggg

SEQ ID NO: 118 - DNA, 584 bp, Mus musculus
  misc_feature (12)..(582): n = any nucleotide
gacctggctttngcncgcnntncanccctagtcccccgggttctcactccctaagcccatcgcagnccggntngtgganccgcgcgtcccaggnttcgtccttncncggcctncaagaacatggcttgcttcagaaagaaaatagttttgtcttctctaanaacttacnttcagcttgtcgaagatgaaaataaaaagccctggagaggaataatttcttgcnctttatgaatctatttttaaaataaaaaaatttaccnnctttnaatctttttcctcctcncaaaagnaaccagtatttttgcctnccattcantttgcnncantaaganntttggagcctgaaaccnnagnctttntnanggantntcnccttggttcagcctgnaggcaaatctgatcaacggacctttatgagtcatttttcctagacatattcagaaaacctaggagctgtgtcaaatgcctgaattaagcattacaaatgcaagatntttgcnctcttgaagaatgtagagagtaaaagaactanaattaaaaanaataangcntgtgatataacggaatatatatntnaaaaanaa

SEQ ID NO: 119 - DNA, 698 bp, Mus musculus
  misc_feature (8)..(686): n = any nucleotide
gccgctgnaggangggacccaatnctctattancatctgnnctgattcttngggcacccngaantttatatncntatcctccnaggatntggaattattacacatgtcgtacttactactncggannaananaaccttncngctatanannantntgtatgagcantantgtcnatngnctntctaggcctttatngnntgagcccaccacatatttacagtangnttagntgtanacacacgancntactttacatcancccgntntnatnatcngcanttnctaccggnntcaaantntttanctgactngtaaccctacacggaggtnnnnttaactcgatctnccagctatacnantgagcctttangctttatnttcttatttacngtnggnggtctaanngnaanttgcntnanccnanntnatccccttgancatnncncattcacngaaccannctanncagtatccnccatttnccnctnttgnncctatcanntggggagtcagntgcnctgctattcntatccagnnacnntgtnttcatctngaanntcccnnttnntntnttcanngccttnccaccccttnttataggaccnccntcnnacncncncaagncntcnacttttcncttnnctcantatctnccntnanggcntctctcancnccnttnntccntttctctttccc

SEQ ID NO: 120 - DNA, 753 bp, Mus musculus
  misc_feature (11)..(750): n = any nucleotide
gcaaacatggncaggagcatcttggcagcnttaagccttcanagaattatcaaccanggncatnagagncgactctgnctcnttcaggcttgcaccaccaccacaggcntaggggccagcacaggcagggtcacttanagagctgagacaccacagcaagngaaggctngcacctttcacttgcccagaggangctctgacnaagggggtgcatcaaccanctccngtgtaagcngnctaaggagtccgaggcagcccccancagctgctgctgtcactgccgccacctcatatttganaagtcanggtctgcantatgcttgacagtntgcgnaaaacctcccatccttatgtanctgacaggngcttttncgcgnantnacaaaagccaccttgaaccctgtcantnctaggtcaccttcnagcttgcctngacnnaaantcnngtccnttgaaacccccnntggcangcccaaaccccannntgaagcttnagatnctcaggacnncnnaanttanannntgnccatttcnnaccttannnttaatttnaannctaaggncattactcttnttccccccaaacntntaacnccttananagnaccnccnnctctttccaattnnttntnnccaaatngtntntnnaccaggtcccccaantctcaanntntaaatttctnnctgncaaacncttananantangntttccctnnccnaacaanttntgnncanactntntncggngnccc

SEQ ID NO: 121 - DNA, 690 bp, Mus musculus
  misc_feature (31)..(690): n = any nucleotide
gttttgtttgaaacggcaacacttcggtcangatatcttctaccagacaccaaggcgtatggagatagaatanaaagaatgcttcgcctcagnttaaacattgaccctgaagcacaggnggaggaagaaccngaagaagagcctgaagacacctcagaagacgcanaagactcagagcaggatgaggnagaagagatggatgcaggnacagaanaagaagagngaggaaacanataaggaatctacnganaaggatgaattgtaanttatactctcgctatgaatcccgngtggagaggnaatgngaagttntgaagtcatttcttttgagagacttgntttgnatgcttccccnngcctccttctcccctgcnctgtnaaatgnttgggattntgggtcacaggaagaagtgnnttttttanctgnantntttttnncattcctcctgaatgtanattnngtnctatttaactgactattggcgtcnnaatcttgtcntgtgtntnaaccctccccanncatccccanctccccnacntnccctccncccctccnccctctctctcnnctcccctccnngnncncnccccncncatcttcntnnacnngngnctncccnccnnntntnctncccnntgcnctctctanannnggggnccccctnaattccntattancntgnccccccn

SEQ ID NO: 122 - DNA, 558 bp, Mus musculus
  misc_feature (14)..(131): n = any nucleotide
ggaaaggacgcggncccccgggctgcgccgggtcgcggcggnccccgcaggcagccatggcggcgggcgccggggccaggccggcgccgcgctgggtaaaggcgctaggtgagccgctcagtgccgcgcnnctgcggcggctggaggagcaccgctacaccgcggtgggagagtcgctgttcgagccgccgctgcagctttactggacctggctgctccaatggatcccgctctggatggcccccaacaccatcaccctcatcggcctcgccatcaacctggtcaccacactagtgctcatcttctactgccctacagtcacggaggaggcaccatactggacataccttttatgtgccctgggactctttatctaccagtcactggatgccattgatgggaaacaagccagaaggacaaactcttgctctcccttaggggaactatttgatcatggttgtgactctctttccacagtatttatggccatcggcgcttccattgctgttcgcctaggaacacatcctgactggttgtttttctgctctttcgttggga

SEQ ID NO: 123 - DNA, 568 bp, Mus musculus
  misc_feature (11)..(562): n = any nucleotide
ctttttgtttnggtgccactatagaatagggccctctagatgcatgctcgagcggccgccctttttttttttttttttttttttttttttttttttttttttttttttttnggttaattggctgaaaattttataacatttaactaaaaattaaacatgnttcatatctcctttcatgaaacagcagcagcaagagatggcaaatgttcgaaagnctnttcaatccatgttattntgatgagtntttgaanaaagaacttgaacctgttccggngcttgttgacatgaagtnttgaanatgtttaaatgaaggngtnttgaaatttgcaggcccaaagaagtaaagtntntcatcatatcaaatgaanaaatgaccattgntntccncagaacaacatattcatntataaaattattaaagnattggtntaaaaanaaaanacctggcccaanaaanacagngtnttgaananatagttcacttttcgtcatgggagctatcaccaatttttgngcaactttagcaaagacncntccaaacnttaaagtntaaaggcanggntgtttt

SEQ ID NO: 124 - DNA, 451 bp, Mus musculus
tgctggaaaggggagatcctgagaacagaccagtggcctcggaggctcttcatgcagctcattccgcagcagttgctgaccaccctcgtgccactgttccggaattcacgcctggtacagttccacttcactaaggacatggagactctgaagagcctttgccggatcatggacaatggcttcgcgggctgcgtgcacttctcctacaaggcatcgtgtgaggtgcgtgtgctcatgctcctgtactcctcggagaagaagatcttcatcggcctcatcccgcacgaccagagcaactttgtcaacggcatccgtcgcgtcatcgccaaccagcagcaggtcctgcagcgaagcctggagcaggagcagcagcagcgagggatgggtggctagaggatgcctgggctgggcgggccacagtcccagacgaggccccagtggagactggtca

SEQ ID NO: 125 - DNA, 718 bp, Mus musculus
  misc_feature (4)..(691): n = any nucleotide
gccngcttctcngcatttaggtgacactatagaatagggccctctagatgcatgctcgagcggccgcccttttttttttttttttttttttttgggttaaaaggctttattagggaaacatacaggggcaaggaccatccttgggagacctnaggacgctgtcctccaggttgctgggcaggtacagtcgccaggagcccctgctnagaagcagctgaccagtctccactggggcctcgtctgggactgtggcccgcccagcccaggcatcctctagccacccatccctcgctgctgctgctcctgctccaggcttcgctgcaggacctgctgctggttggcgatgacgcgacggatgccgttgacaaagttgctctggtcgngcgggatgaggccgatgaagatcttcttctccgaggagtacaggagcatgagcacacgcaccttacacgatgccttgtagganaagtgcacgcagcccgcgaagccnttgtccatgatccggcaaaggctnttnagagtctccatgtccttagtgaagtggaactgtaccaggcgtgaattncggaacagnggcacgagggtggtcagcaactgctgcggaatgagctgcatgaagagccttcgaggccactggtctgttctcaggatctcccctttccagcacactggcggccggtactagtggatncgagctcggtaccaagcttgggtcttc

SEQ ID NO: 126 - DNA, 544 bp, Mus musculus
  misc_feature (5)..(540): n = any nucleotide
atcgncccccttggtnnccggctcggaccctntcaacgcccgccagtgtgctggaaagcagcgagcggctgcttcgtggagcagagaggtgcatcaccaggttcccgatgaacccagagaaccctccaccgtatncgngccccgggccaacagccccatacccaccttatccacaacagccaatggggccaatggggcctatgggagccccacctcctcaggggtacccctacccaccacctcaggggtacccctatcaaggatacccacagtacggctggcagggtggacctcaggagcctcctaagaccacagtgtatgtggtggaagaccaaacgnagagacgacctgcgcccatccacctgcctnanagcctgctngactgctctgngtngctgctgcctctgggacatgctnacctgntcanctgatgagcccagctcttccgnttgnccgctctgngccanctccnatncntncncctgnncccatctctcctgcttnctctncagntgcctancctcctcctctccacnctcnttta

SEQ ID NO: 127 - DNA, 598 bp, Mus musculus
  misc_feature (573)..(597): n = any nucleotide
ccttttttttttttttttttttccagattttggacagatttattgaaacataaagggtatgagcagagagatctagtagtgtgtcacatattgccattaccttgagtgtataatttaaacattataaatatatatttcataactaagcctttggccaaaaaagtaaattatttagcacattttttaaagatcagtaagaaatgagttttgaacattaaaaagatcaagtcactgaactaaatagcagtaaccctcactaatctaaaactccataggacagaaagtagaggtgtctgcgcagagctagccactttatagcaatcagaagagatggggccaggcacacttatcggaggtggcacagagcggccaagcggaagagctgggctcatcaggtgatcaggtgagcatgtcccagaggcagcagcaacacagagcagtccagcaggctgtgaggcaggtggatgggcccaggtcgtctcttctttggtcttccaccacatacactgtggtcttaggaggctcctgaggtccaccctgccagccgtactgtgggtatccttgataggggtncccctgaggtngnggnagggggtnc

SEQ ID NO: 128 - DNA, 668 bp, Mus musculus
  misc_feature (10)..(668): n = any nucleotide
ctggaaaggngcggagcctgctgctgccatggaggctggnggctgggtacactntgtctagactgggggccctgggcggctnnngngccgggtngggacngttacttggtactgccgccggccttggattcctgtgcgtcctttacagccagcgatgganacggacccagcgccntggccggagtcacagtctgccnaactccctggactatgcgcaggcttcagagcgtggacgncagnngacacagnttcgggctatcccaggtgaagctgnagatgctgccatactgcccagcctctcacaggaagggtanganaaggtgctggaccgcctggactttgcgctgaccagtcttatggcgctgcngcgcgaggnggaggagcttnagagaagcctgcaaggactagctggngagattgtcgntgaggaccgctctcatatataagagaaccananagaggnccnggcggcgcacgntccctttatgccagagagaggagngactccacgggctncagctctgtctacttcaccgnctcctcaggggccngcactcacagacgccnanagnnagggaggctatncnncagcccaaccgcggagtntgattacnagcngnactncnacaaggaanagtgganatgcntgangaccaaanngaagctnn

SEQ ID NO: 129 - DNA, 695 bp, Mus musculus
  misc_feature (28)..(92): n = any nucleotide
tttgaaagcccgttctagcatttaggtncactatagaattntgacctctatatgcatgctcgagcggccgccgacggactggacgccgcctncacatccaggtccagagagtccttcctccccatcctcacggtctcgcagctcacttcgtcctcagcatctccactctccttgtcggagtcccgctcgtaatcagactccgcgttggctgttgtatagcctccctcgctctcggcgtctgtgagtgcggcccctgaggaggcggtgaagtagacagagctggagcccgtggagtcactcctctctctggcaaaagggaacctgcgccgccgggccactctctggttctcttctatatgagagcggacctccccgacaatctccccagctagtccttgcaggcttctctgaagctcctccacctcgcgccgcagcgccataagactggtcagcacaaagtccaggcggtccagcaccttctcctgcccttcctgtgagaggctgggcagtatggcagcatctccagcttcacctgggatagcccgaaactgtgtcacctggcgtccacgctctgaagcctgcgcatagtccagggagttgggcagactgtgactccggccatggcgctgggtccgtttccatcgctggctgtaaaggacgcacaggaatccaaggccggcggcagtaccaagtaa

SEQ ID NO: 130 - DNA, 597 bp, Mus musculus
  misc_feature (44)..(596): n = any nucleotide
gtgtgctggaaagcgctgggtctgagtgaccaaaggcagtagcnctcgcggagatcacccgctgnccctngatcaccatgtcggccttcgacactaaccccttcgcggacccagtggacgtaaaccccttccaggatccctctgtgacccagctgaccaatgctcctcagagtggcctggctgagttcaatcccttctcagagacaaatgcagcgacaacagttcctgccacacaagctcctgggccctcccagccagcagttctccagccctcagtggaaccagcacagccaacgccccaggctgttgcagctgcggcccaggcaggcttgcttcgacagcaggaagaactagacaggaaagctgccgagctggaacgcaaggagcgagagttgcagaacactgcagcgaatttgcatgtgcgagacaacaactggccgccactcccctcatggngccctgtgaaaccctgcttctatcaggacttctccacggagatccctgctgactaccagcggatttgcaagatgctttactatctctggatgntgcnttcagcgctctgtttctaaacctncttgcgtncctggcctntnt

SEQ ID NO: 131 - DNA, 595 bp, Mus musculus
  misc_feature (18)..(18): n = any nucleotide
tttttttttttttttttnaagtccttgtcccttcctctccttccctcttgtagaggagaaagctgactactgagggaggagcagggacaagacaaatccctgccaggtctgtgctggtgtagtcactgcataaggcacccgaggaaagtgcagctcagaagacagttgacagagaggccagggctcagggaaggagagtcgggaagcctaattcccctgaaaggctcctcgggcagcagatgaggcagccccgcgaaaggttctgctgctgaagataccctgggagaactcttcctgggcctgctggaagctggctcctgttcggcggtagaaggcgtgtacccgctgcaggaggaagagtgaaagcccagcacagagggtaaagaagccagccactaccatcatgatgatggtcacagctaagggcccatttttcatcgtagatagggctgcaagccaaccactggtccccaagttaggcaagccaatcaactggatgaagtagatccctatttgacaaaaaaatacaaaaaagaacacgaagaagctgaaagagttgtcagacctgaaagccttgtagatgggtcggtccaac

SEQ ID NO: 132 - DNA, 829 bp, Mus musculus
  misc_feature (780)..(803): n = any nucleotide
gtgtgctggaaaggcagcatctattcgtcactgccaggctgaactggagaggctttcagagtccctgaggaagagcatctcctgtggagctttctctgttcctgggggtcacagcctctacttagaagccaggaagaagattgagctgggctaccagcaagtgccgaggaagggagtgaaggcaaaagaggttctcaagagtttcctacagtcacaggctattatggaggactctatcttgcagtcagacaaagctcttacagatggagagagagccatagcagctgagcggacaaagaaggaagtggctgagaaggaactagagctgctgaggcagagacagaaggagcaagagcaagtgatggaggctcaggagagaagcttccgagaaaacattgctaaacttcaggagaagatggagagcgaaaaggagatgctgctgagggagcaggagaagatgctggagcacaagctgaaggtccaagaagaactgcttattgaaggattcagagagaaatctgatatgttaaagaatgaaataagtcacctgagagaagagatggaaagaacaagaaggaaaccctcactgtttggtcaaatccttgacacccgttggcaatgcgttcattatgattttaccaggagctggtaaactatttggtgtggggctgaaattcctcggctcactaagtagttagtctaaagagtttctgggaaggaatttataaaatccagcttctggtattttgataccaaactttttgctgggatttttaagtncacattctagtttaataaccacngttaccaagaaagtaagactttcatt

SEQ ID NO: 133 - DNA, 592 bp, Mus musculus
  misc_feature (92)..(469): n = any nucleotide
ttttttttttttttttttttttttttttttttttttttattttaatctgcatgccttttattatcaatcaaacattttcctcatacattctnttatgaaaacccacttgngcgttttaacaagagctctttttaacaatttatctgtagtgtacaactgggatacatatgccaatgngtcttcatttggccatgtttctgnttgatttacttacatcttttctagaaataatttcactatattctgactggcctccctctttattatttagataacatcactgaaatttgacaggatttatggacgccagacccttatattcttcaatcattggtttgnatctttaaccttaaatataataggnaatgaactgacacagaattagactccgaggaagcagtatctgacaatttagctattaatggaagttcttactttctttgtaaccgnggttattaaactagaatgngtacttaaaaatcacagcaaaaagtttggtatcaaaataacagaagcctggattttataaattccttcccagaaactctttagactaactacttagtgagccgaggaatttcagccccacacc

SEQ ID NO: 134 - DNA, 874 bp, Mus musculus
  misc_feature (9)..(852): n = any nucleotide
tcgcaccanccttggaccgagctcggattccctagtaacggccgccagtgtgctggaaagaacgcctctggggagcatggcatcgtggttttctctttgggatccatggtctcagagattccggagaagaaagccatggaaattgctgaggctttgggcagaattcctcagacggtcctgtggcgctacaccggaactagaccatcgaatcttgcaaagaacacaattcttgtcaaatggctaccccaaaatgatctgcttggtcatccaaagactcgggcattcatcacacactctggctcccatggtatttatgaaggaatatgcaatggagttccgatggtgatgatgcccctatttggcgatcagatggacaatgccaagcgcatggaaactcggggagctggggtgaccctgaatgtccttgaaatgactgctgatgatttggaaaatgcccttaaaactgtcatcaacaacaagagctacaaggagaacatcatgcgcctctccagccttcacaaggaccgtcctatagagcctctggacctggctgtgttctgggtggaatacgtgatgaggcacaagggggcaccacacctgcgcccggccgcccatgacctcacctggtatcagtaccactccttggatgtgattggcttcctcctggccattgtgttgacagtggtcttcattgnctttaaatgttgtgcctatggctgccggaaatgctttgggggaaaggggcnagtgaagaaatcacacaaatccaagacccattgagaagtggggggaagtgaangagaagtattagttcattatctgacagttgaactttggnaacaagtgttnganccataatggtttgttaggggaa

SEQ ID NO: 135 - DNA, 588 bp, Mus musculus
ttttttttttagtgatacaatttatatgaatttattgataagttcttggtttgggaacacaatagaagatgtacttgccttagaacatactttggttttcatcaaattccggcacaagcaacaattattatctcaaacacagggccatcagtgtcacagcctcatctcttctcccaaagctccaaaggtcttgctacccataatgcatgaacacaatgaagaatgactaacactggagaaggccacggcagtctgagaatccaggggagaaaaccatcagctggcgggagcaggggaaactgtggggctgtcactgaaagcagagtgccagactttctgtaaattaccaacagatggcaagagttgtctagactgaagatactggaaaagcacatcacatatactagaaccttccctgatgggtgtggccagcaattagattgttattattattatttttaaatgtgctgaatgtataatgtatggtgaattatttccctaacaaaaacaatatggatctaacacttgtttccaagtttcaactgatcagataatgaactaatacttctccttcacttccccccactt

SEQ ID NO: 136 - DNA, 832 bp, Mus musculus
  misc_feature (670)..(817): n = any nucleotide
gccagtgtgctggaaagagaagcagacatggctcaatttgcacaggtcatggctgaagtgggcgactttggtcgctttcaggtgcggttgaccatcctgatgggcattcccaatttcctggctgcattcttcatatttggccaagtcttcatggtccttgatgaggctcaccactgttcagtgtcctgggttaagaaccacactttcaacctaagtgccgctgagcagctggctataagcatccccaacgacacagcgggcagacccgagtcctgcctcatgttccggccacctcctgacagtgccagcctggaagacatcctgagccaccgcttcaatgagacacaggcctgtgactcaggctgggactatcctgagaaccggcctcagtccctaaagaaagagtttgacctggtgtgtgatcgaaagaacctgaagaagacctcccagtcggtgttcatggctggtctccttgttggggccctggtctttgggcctgtctgtgactggattggccgcagaccctccctcctgatgcaggtgcttctgtcaggcatcacaagcatggccacagccttcgtgtccagctttgagctctacttggccctacgctttgtcttggctactgccaatgctggatttttactaagtaccaatgtcctgatttcanagtgggtggggccatcttggagaacacaagccgtggnctttggcccanagcaacgttgcccttgggcagatggtgttagcaggactggcctatggtgtccgaaacttggagacttcttcagatacanggacccgacccgcttactgntcttcttctatttct

SEQ ID NO: 137 - DNA, 813 bp, Mus musculus
  misc_feature (11)..(812): n = any nucleotide
ttttttttttntngatattagntangttttattatttnttatctntatgaggaaggggtatcccagacagggagactgntgaggnnacaatcctagagagantgtcngagaagactganagcgaccctgactcanccgtggtactggggtgcagttngtgtatganaagtgtggaanagggaagggtactgtccctgatactccctctgggctctgcaaaagggagtagggagcatgaacacggctgatctgatcctgtgngctanagatgccatgtgcgctgcctctgctcttgataggctggncctcancagccacagtcanaantncganctcttcacaacancnacnctcanactnnaagatcttcccaaagcctncangtcttgnctnngnnntgcngatnngagggaccctgcaaggagcccctgntcnagganctggaggntgtnctncagggtcnggccctgggtctcaggaagtagagcgnacagtangcctgctncaatggngaggctgcnaaagatgacnatgggaatcgnctggtggactgttctagtagcatcacaangggtgtgatgatgccaccgaccctgtagaagatgctnaccaaccccatgcctgtttgcctgatnatggatggtngangagctnagctagtgnacacatangagatggtgaangccgcaaccgagncaanatttcncnatcataggcagnananaggcncaccgngggangaactcctgggatgnaaaatataatgccgnccatgancccgnctgggacngcantcnacagnttgttttcnna

SEQ ID NO: 138 - DNA, 739 bp, Mus musculus
  misc_feature (665)..(725): n = any nucleotide
ggaaagggagcagcaaacggccggcggcaggcgccgcgcggggggcgggcggcgcggaggcggcggtggccatggccgaggcgtcgccgcagcccggacggtacttctgccactgctgctcggtagagatcgtgccgcgcctgccggattacatctgcccaaggtgcgagtctggcttcattgaggagcttccagaagagaccaggaacacagagaatggctcagccccctccacagcccccaccgaccagaaccggcagccatttgagaatgtggaccagcacctgttcacgctgccacagggatacagccagtttgcctttggcatcttcgacgatagctttgagattcccacgttccctcctggagcccaggccgatgatggcagggaccctgagagccgacgggagagagagcaccagtctcggcatcggtacggggcccggcagccccgtgcccgcctcactgcccgccgggccactggccggcatgaaggtgtccctacgctggaagggatcatccagcagctcgtgaatggcatcatctctccggctgctgtgcccagcctgggccttggtccctggggcgtcctgcactcgaacccaatggactacgcctggggggccaacggcctggacaccatcatcacgccagctccttaatcngtttgagaacacccggnccccccacttgcagacaangagaagaattcaggctnttccccncggtcccagtcaca

SEQ ID NO: 139 - DNA, 1260 bp, Mus musculus
  misc_feature (29)..(1259): n = any nucleotide
tttttttttttctgcttttttgagtaggngtattttaaatagctttccagataacacacatttcccttaaaaaaaaaaaattgtctgctggagcagtttgttcctgggtctgctggtcacccttgnggtcccgaggccggcgctggttgggngctgggcccggcctcccagggcgtgtggcgggctgggcctggcgacagttctgctgantctccacagaccatgcatggccgnacgtggctcaaacgtccatttatttcaaagcagtaataatttaaaatcataaaaacccttctgccgttgaacatttggagggtgaggttaanacggactgaaggtgcctgcctgctggggacatgggacccacgtgcacacctccaaacaggagcggccatggccaagtccaagcctgcccgnagcctggggcccgccaggnactgccggccgccacgggcagcacctctagtccacagccatggcgggcgggcaggcggncggtgctggggcacctggggtgggtgggatgaccaaaccccgggggggatggncaggtgggactcaggagtttgcttgngggcgttctcattgctgggcnaaactggaggaggatgaangaccaangactaangaggaaacctacaccgggcaaagccttggggggaatggncggcttgggtnctgtccnagtgaggctntttacggcaaanccgggcaagcntggcatgctgnnttcagccangggcacgaatccncntgtcgtggnaaaaggtgggntgcaagggcnaanttngccggnanactttttacccagggggaataaanncttnttttaacnnactgggncaacttaaagcccttggagccccncgntggttcctnctggggncttngggacccntnnnggaanaaaccttgaaanttctcccttgnnntgcancttggggggccccngggttttcnaancggaatgngggactttcnatnaaanaaggggnnccagccnttgccccccaggcggaaacccantnggnttcaagtccggaacccccnngggccaaggcccanntgnccaaaaaccgggaaaaaagcnttncaaaatntnggagaaccttcccgcnggcccctttttncccaaggncccnggcaangngcaccgntnttnggccctcccaaccaannggtttttctcgnctnaggcttnntaaanncggctnccggnaangggaaanaaannntaaaccncgcnangtttttnttnnc

SEQ ID NO: 140 - DNA, 591 bp, Mus musculus
  misc_feature (301)..(590): n = any nucleotide
agtgtgctggaaagcgggcctgagccgagcagctgcgcgacgtcatggacaactccgggaagcaggctgaggctatggcgctgctggctgaagcggagcgcaaggtgaagaactcgcagtccttcttctccggcctctttggaggctcatccaaaatagaggaagcatgcgagatctatgccagagcggcgaacatgttcaagatggccaagaactggagcgctgctgggaacgctttctgccaggctgcccaactacacctacagctccaaagcaagcacgatgcagccacctgctttgnggacgctggcaatgctttnaagaaagctgacccccaagaggccattaactgtctgatgagagcaattgagatctatacagacatgggcagattnacaatcgcagccaagcaccanatctccatcgctgagatctatgagacagaactggtggatgtagagaaggccatcgnccactatgagcaatctgcagactactacaaagganaagagtccaacagctnanccaacaagtgtctgctnaaggnggctnctacnccnnacagctggagcagncccanaaggctatcna

SEQ ID NO: 141 - DNA, 351 bp, Mus musculus
  misc_feature (38)..(350): n = any nucleotide
tttttttttttttttttcatgcaaaagtggcaggtttnattgtcctttttgggccagctgnagcttnaggtcgatagacctggatgcatggagagaagcaggtgcgcagggcagggccaaggcaccccatgagctccagggctgggtctaacctgagaggttggcattgnaggaacaagggaaggctccaggggcagaggttacccccagccagggagagnaagtntcagagctaccngngtncttatctctgctaggggacaaggtaggagatatggaaacagncttagtacnctgctgntnaggagatgntnccannccttganctncggtacntnaagcacacagant

SEQ ID NO: 142 - DNA, 928 bp, Mus musculus
  misc_feature (49)..(906): n = any nucleotide
aaagttgttcttctcgtggttcccagtggcgagaggaggaggaagcccngagcggagcggggcggctggggggggtggacccgccgcggctgctgctgccaccgccgccgccgccaccaccgctcgtggggctcgtggcgtgaggaaggaggacgagtgagaccccggggcgagcgggcggcggcgccgctgctgctgctgctgctgcgggagggncggcggcgggacggcgatggcggatatcgacaaactcaacatcgacagcatcatccaacggctgctggaagtgagagggtccaagccaggcaagaatgtccagctccaggagaacgagatccgaggactctgcctgaagtctcgggagatcttcctcagtcagcctatccttttagaacttgaagcaccactcaagatatgtggngacatccacgggcagtactatgatttgctccgtctgtttgaatacggtggctttcctccagagagcaactatttgnttctcggggactatgtggacaggggcaagcagtccctggagacaatctgnctcttgctggctacaaaatcaagtatccggagaacttctttcttctcagagggaaccacgagtgcgccagcatcaataggatctaccgattttatgatgagtgtaaaagaagatncaccattaagcttgtggnaaacgttcacagactggtttaactgcttgccgatagcaagccatcntggacnaagaaganaatctgctgtcatggaggttatcaccngaacttcaatctatggagcngaattccgcggaaatattagaccanttgangtaccngaacaagggccttctttgggaaccnttgggggcctganccccataaagaggcnttaagcttgggtggaaaatgcccgaggagnggccttccaattggtgccaaat

SEQ ID NO: 143 - DNA, 1017 bp, Mus musculus
  misc_feature (309)..(1015): n = any nucleotide
ttttttttttaacaagcttgcatttaataagtctgaaaccattctcagcacatggcattgtacacgggcatctgtgcaaacagattcatttaacaggtcgtagtttaaaaaagtcatagatactgtgagttctgtataaaccggtggacggcaagttagttccttttgatttataagcctcaatgtcaccgcagaataaagaatgtagccaaagaaagcattatcggtcactcgtataggacagtgttgtttctataatttgaagctttctgaatggacgggttcaggcctgatccaacccgtggtggngtgacaggtctcgtggcgntgggcttctttttctctgcaggctttaaaatctggaaggaacacatgagggtctcatccacactcatcatggcgcctgcattgtcaaactcgccacagtagtngggtgcagaaaacagagtgactaactgcctctttgcaaaaaactcatagccatcttcaaccacctgatgggctctacatataagatccaaatcatgcttatggagaaattttgcaaccacttctgcaccaaatgtgaaaggacacttcttctgncaattttcaccccagnctaagacatcttttaatcggggtcaggaccnccaaaggatcccaaaggaagaactttgatctggtacatcaagttgggcctcanaaattcgnccggaanctggnttccatagaattgaagaactgggggaanaaaccttccatgacaggcngaaaaatcttnttngtccccccaanggcttgntaattgggcaaggcnggttnaaaacagtcnttgtgaaccgtttttccncagcttnaaagggtgggaaacttctttttccccttcatcattaaaaaaacccnaggaaacctaatggaagcttgggcgccaaatcgggggtnccctttttgggaaaaaaaggaagntttttccgggaaacctttggattttttagggnccccccaagnngggcaaaattggnttnccngggnnct

SEQ ID NO: 144 - DNA, 831 bp, Mus musculus
  misc_feature (21)..(818): n = any nucleotide
gccgccagtgtgctggaaagngagactcggcgatactgcacttcctcagagcttgctgcccactactgcaagaagtacaacatccagtaccaggagagcttctatgcctggagcaccccaggcgtgggcaagttngtgacttccatggctgcctcagggggcatctatctcaccctgctgttcctcattgagaccaacctgctgnggcgactgagaaccttcatctgtgccttccggaggaggnggactctggcagaactgcagaaccggacatcagagctgcccgaggaccangatgtagctgangagaggagccgaatcctggtccctagcttggactccatgctcgacacaccactgattatcaacgagctctccaaggngtatgaccagcgagcaccgctccttgccgnggacaggatctcccttgcggtccagaaaggggagtgcttcggcctgttgggtttcaatggagctggaaaaaccacaacattcaaaatgctgactggggaggagaccatcacctcaggggacgcctttgttggtggttacagcatcagttctgacattgggaaggtgcggcagcggatgggctactgcccccagtttgatgcactgcttgatcacatgactggcagggagatgctggttatgtatgcacggctccgaggcatcccanagcggctcatcaatgcctgtgtggagaatactctgcggggtctgctgctggaacccgcacgccaacaaactagtcaagacttacagtggtggtaacaaacgcagctaagcactggcattgcctcattggaaagcctgcngttatcttctgga

SEQ ID NO: 145 - DNA, 811 bp, Mus musculus
  misc_feature (33)..(807): n = any nucleotide
ttttttttcatgtccccttatcctttatttttnatanaatcacaatatgtttaagngtatcctcaananatgacactactcagccctgnannggtccctgacaatcctgnggccagngacttnttgaaggcacanagctaggaacagaggatgngcttcaagtctggccacagctctctcttgngctgccgatgctctgngggatgacatctggctcaggatcagatcagccgggtccactgtccatactggaaggaacattgacatccaaggatgcctntgcagttgcattgggagccaacggtctcagcacctagtcatagccagtatcttcccttcatgaaggnatttgcaactttgctgctcattcctggcagcctggaagtcctcattgctgctnaaccctgtnggctttcttcccccccccccnaggggagcttagngtcatatgtaaacatagacaggtccctggggnctctctggataancactggaaagaagctcaatccgccaggctagctgcagaaccagctgggacagttccccatccaaagactaaggcctgtctgtgtgcacagcaggatggggnctgggactgnggtccaganacctgacttcaggagtggaaacctgcatgctgactcctgcctgcaggcactgacagacacacatggtccactttgngcatatcaccttgantggttttcatgnccatcatagaaaagagtgattaaaaatganngaaaggagaaaactgggagggaaaggacccacgggntaagcncgaaatngccccttncacgcccngggg

SEQ ID NO: 146 - DNA, 703 bp, Mus musculus
  misc_feature (3)..(388)
  misc_feature (3)..(3): n is a, c, g, or t
cgncacccccttggtaccgagctcggatccctagtaacggccgccagtgtgctggaaaggatcatccgtctcgcctccagttcttcaccggctgagctgggtacggacatctcagtgaccacgagctaacatagcacgtgaaactgacctcaggaagaacgaagcctggagacccggagtgcgacagcgacctccggactgaagtggaagaagcgtagcgaggccttcgtgtcctcgctgtggggggcggggcttgttgtgcaggcggggctcacgtggtactttggtactttggatgactgaagggaagcttggtacccgcctggtcactacccggaagtccagccggagtagcactaccctggagtccacccggctgtgaagcggnggtgctcttaggggtgcttgacgccctcattgttagaaactgatgggtactgttggccgattcctagacccgcttttgacctacgtgataaatgtgtttccggaactctgccaaaaagtgttctggaggtttttgtttgtttcggagccaaggcctccctcgtagctcttaatggcctgaaacactctttgtagcctagactggcattgaagtcaagttagccctgcctttgcctccctgggctctgggattaccctgcctttcttgattatttctatttgaacaaatatgaacaaactatgtggccaccacaaa

SEQ ID NO: 147 - DNA, 604 bp, Mus musculus
  misc_feature (17)..(595): n = any nucleotide
ttttttttttttgtggnggccacatagtttgttcatatttgttcaaatagaaataatcaagaaaggcagggtaatcccagagcccanggaggcaaaggcagggctaacttgacttcaatgccagtctaggctacaaagagtgtttnaggccattaagagctacgagggaggccttggctccgaaacaaacaaaaaccttcagaacactttttggcagagttccggaaacacatttatcacgtaggtcaaaagcgggtctaggaatcggccaacagtacccatcagtttctaacaatgagggcgtnaagcacccctaagagcaccaccgcttcacagccgggtggactccagggtagtgctactccggctggacttccgggtagtgaccaggcgggtaccaagcttcccttcagtcatccaaagtaccaaagtaccacgtgagccccgcctgcacaacaagccccgccccccacagcgaggacacgaaggcctcgctacgcttcttccacttcagtccggaggtcgctgtcgcactccgggtctccaggcttcgttcttcctgaggtcagtttcacgtgctatgttagctcgnggncactgagat

SEQ ID NO: 148 - DNA, 872 bp, Mus musculus
  misc_feature (748)..(848): n = any nucleotide
ttcaccccgcttggtaccgagctcggatccctagtaacggccgccagtgtgctggaaagatggagcctgcgtttcacagaggggatctccttttcctcacgaaccgagttgaagatcctatacgcgtgggggagatcgttgttttcaggatagaaggaagagagattcctatagtgcaccgagtcctgaagatccatgaaaagcaagatgggcatatcaagtttttaaccaaaggagataataatgctgttgatgaccgaggtctctataaacaaggacaacactggctggagaagaaagatgttgtggggagagcaagagggtttgttccttacattggaattgtgacgatcctcatgaatgactatcctaaatttaagtatgcagtactgtttctgctcggtttatttgtgctggtccatcgtgagtaagaagtcggactccctgttcctaggaagctgctgtgcttgttgttactgaatgttggagtagatcctgatctgtgattgcggattttcggaggacacacacgttggcacttcttggtagccctggtttgcattgctttgtgtttccacaccagaggctgtgtgggcgggtgcatgtgcaccgtggagtgcacacaaggggactgtcaatcacagggtttcatatgttgtcattgtcactctttcacatttttgtcatcagtgaattttttatattaaaaggttgagccaaagcccccagtgtttgtattttgaagccnagcttcactttaaaagtgcctacagagttctgtaaatgaaaacacagctctgcatgagttcaaacctgncggtccttcttacagtaggaatggcncatantgaggcgggcataagtcttacttt

SEQ ID NO: 149 - DNA, 813 bp, Mus musculus
  misc_feature (17)..(785): n = any nucleotide
tttttttttttcaatgngcaaagtcttttatttaaaattttgaaaagttaanacttatgaccgcctcaatatgngccattcctactgtaaggaggaacgacaggtttgaactcatgcanagctgngttttcatttacagaactntgtaggcncttttaaagngaagcttggnttcaaaatacaaacactgggggctttggctcaaccttttaatataaaaaattcactgatgtncaaaaatgtgaaanagtgacaatgacaacatatgaaaccctgngattgacagtccccttgngngcactccacggngcacatgcacccgcccacacagcctntggngnggaaacacaaagcaatgcaaaccagggntaccaanaagtgccaacgtgtgngtcctccgaaaatccgcaatcacagatcaggatctactccaacattcagtaacaacaagcacagcagcttcctaggaacagggagtccgacttnttactcacgatggaccagcncaaataaaccgagcagaaacagtactgcatacttaaatttaggatagtcattcatgaggatcgtcncaattccaatgtaaggaacaaaccctcttgctntccccacaacatctttcttctccagccagtgttgtccttgtttatagagacctcggtcatcaacagcattattatctcctttggttaaaaacttgatatgcccatcttgcttttcatgggatctttanggactcggngcactataggaanctctcttccttntatctgaaaacaacgatnttccccaccccgtatagggatctttcac

SEQ ID NO: 150 - DNA, 707 bp, Mus musculus
  misc_feature (6)..(705): n = any nucleotide
cacccncttggtaccgagctcggatccctagtaacggccgccagtgtgctggaaagntccttgcttcttttttggggtngcggccttgctcctgtcctgcnagagaancntanagtgntttnacgcnnaagtgnncgcgtnggttcngangtnntcntaggnccatgctccagactctnnngtgacngaagcncnanccntgggcnntacnngtnncnccnnnnggntnatcttncaggcnnncnataangagaacatnaccgtgganaangnnatccgnctttnagaaaatgtnatcnaccggatgaaggaggtcctctccatctggctctaaggntnngcnatattcnagcgtntatgntgcttnagnttctgatgaagattntaaaagaagagtagctgncgagctggcnttggagcaagcnaaaaaggagncactgcaccaganacnccttangcaagcaaggggacctataaaagagaaagggctgcatccaatgagcagctgactagagctgtcctttnggaaagaatatctagcnaanaggagcncatgaangcnaancntctggctcnncnnctggaagaganagatngagngatgccggaancagnntgntggagannngaggctnaagttaccctganaacatgcaagctccttcaacgntnattccacaggtggaacatttttcttttcctagnac

SEQ ID NO: 151 - DNA, 607 bp, Mus musculus
  misc_feature (19)..(605): n = any nucleotide
ttttttttttttttttttnttntattntttttctagnctaaatggtacctttattaggtgccaggnanggaantnnctataatnanntanntgnatnancaanccnnngtngnctttnnattgncaaaaaagccnnanantacnngtaatanaaaccnttantcnannanannangcgcctntttnctncnntggggcttncttgaaanaacattatggnnngccccgaatnatntnntgtcnaangaaaatancatanccnggnttnncaggnnnnntaggaanacactgnnggccnaacccatagctntnttgntggntgtncatcnnaaanacgnttaangatnctnctnccagctttgtcaaagnggaatgtccctnaangctggaaggagcctnaagggannggannggnnaanatgccaggangctnnaaannnngtanatgaaaggngnnggggncngncttncnntntnatncanttgggggantgttaacaggttaacagtgncgtttgactctgncannngntttaaagnangggttttaancngntgntnatggcacgttctgaattantggngtccatnttccagngcctgtntg

SEQ ID NO: 152 - DNA, 754 bp, Mus musculus
  misc_feature (242)..(752): n = any nucleotide
aaagttcttctctcacgtgggttggctgcttgtgcgcaaacacccggctgtcaaagagaagggcggaaaactggacatgtctgacctgaaagccgagaagctggtgatgttccagaggaggtactacaagcccggcctcctgctgatgtgcttcatcctgccacgctggtgccctgggactgctggggcgagacttttgtaaacagcctgttcgttagcaccttcttgcgatacactctggngctcaaacgccacctggctggtgaacagtgccgcgcatctctatggatatcgcccctacgacaagaacattcantcccgggagaaatatcctgggttccctgggtgccgngggcgannggcttncacaactaccaccacacctttccttcgactactctgccanngagtaccgctggcacatcaacttnaccacgttcttcatcgactgcatggctgcctggcctgcttacgaccgnaagaaagtttctaaagctactgtnttaccnngattangagaactgnagacnggagtcacangagtagctgagctttgggcttttgagatcctgttttaacggtttctgncagaganttaanattctgtgantaactaacactggatattgntnaatangggggtaangatgctttaaccccaatcnnggnccgnattctttataaaangagaaannctttttnataccccnttgagggggnaaaanaatttnntttnncctngggntaanccntg

SEQ ID NO: 153 - DNA, 797 bp, Mus musculus
  misc_feature (32)..(793): n = any nucleotide
ttttttttttttttttttttttttttttcctntntgnanaaaaggttcaaattnatngtnatnattaaaagcngtngggcaaaatngnanangcattnannangggggctnaaaaanananaaggnntnggggcanggtaaatcctagntaaanaaaanattnattacctcaaaangggatcaaagntttntcatttnanaanaanacngnnccggaanaggtaaaaggcatnattaacnccccganagcaanatccagnngttagtnaatcancanaanattaaatnttngccaaaaaacgttngaaanaggaantnaaaancccaangntaagntantntngggantcccgtctccagttntntaaanccnggntaaaanagnanccttaaaaanttttttcnggncgaaagccaggcccagggcagccatgcagtcgatgaaaaacgnggggaagtngangngccannggnactnantggcagagnagtcnaaggggaaggggngggggaagttggggaagccntcgcccacggnacccagggaaaccaggatattntcccggnattgaangttcttgtcgaaggggcganatccatanagatgcncggcactgttcnccacccagggggcgttgagcnccagagggtatcgcaagaaggggctancgaacaggctgtttacaaaagtttcgccccannagtaccagggcccancgtgggcaggatgaagcacattancaggaggccggcttggaaaacctcctttggaaaataccagttttcgggttnnaggc

SEQ ID NO: 154 - DNA, 686 bp, Mus musculus
  misc_feature (214)..(677): n = any nucleotide
aaagcagctgatctcatacaaacaccccagacctccagcagctccaggagtcaaaaacaccagcagtggaccgaggtcctacagctgcctgcagccatgttggccttcagtctgctcgtgctgggtctgctcgcggaagtggcacctgccagctgtcaacaaggtctaggaaaccttcagccctggatgcaaggcctcattgctgtcgctgtgntcttggtccttgttgcaatcgtcttcgccgtcaaccacttctggtgccaggaggagccggagcctgggagcacagtgatgatcattggaaacaaggcagatggggtcctggtgggaatggatggcagatactcctcaatggcatctggttttaggtccagcgagcacaagaatgcctacgagaatgttctggaggaagagggcagggtccgcagcacacccatgtgacaagcttctctgtggccctagtccccaggctacagggaacatagagttgatccccaccatctnacccagccactgctctacaggaatctactgaaacaagctgacttcctcactctctagaatcacagncatcttaggccaggactnagaccaagcccagcacaccaatgtntgtgacccctggctnntctnggcttntacccagtctgaggagcctttnnnaaggnggttaactncttcccttc

SEQ ID NO: 155 - DNA, 297 bp, Mus musculus
  misc_feature (22)..(294): n = any nucleotide
ttttttttttttttttttccanaagtataaaaagtcttnatttcacagaaataagagccanttccatagttgcaggagcaagggnaggggaggagctgagccacccttcacaaggctcctcagaactgggtagaagacccaggaaagcccagggtcacagcanatggngngctgggcttggctgagtcctggcctgagatgactgtgattctagagaggtgagggagtcagcttgtttcagtagattcctgtagagcagnggctggntgananggtggggannaacnntangnnccc

SEQ ID NO: 156 - DNA, 919 bp, Mus musculus
  misc_feature (668)..(915): n = any nucleotide
aaagcgctcctcgagggtcggtcccgccacgtctcttcccggtctctctggctctggagtgctttccccccgcggagggtgggagcgcggggacagacgggcgagatgagcaccatgtttgcagacacgctgctcatcgtctttatctccgtgtgcaccgcgctgctcgccgagggcataacctgggtcctggtttacaggacagacaaatacaagagattgaaggcagaagtggaaaaacaaagtaaaaaattagagaagaagaaggaaactataacagagtcagctggtcgacaacagaagaagaagatagagagacaagaagagaaattaaagaacaacaatagagacctgtcaatggtacggatgaaatccatgttcgcgattggcttttgttttactgccttaatgggaatgtttaattccatatttgatggtagagtggtggcaaagctccccttcactcctctgtcttacatccaaggactatctcatcggaacctgctgggagatgacaccacggactgctccttcatcttcctgtatatcctctgtaccatgtcaattcgacaaaacatccagaagattcttggcctcgccccttcacgagctgccaccaagcaggctggtggatttcttggcccaccaccttcgtctgggaagttttncttgaaggaaagcagaattctgaatttcctgncatactttttagacattcacatcagacttaccgagcacctggccacaatctaggtnggggtaatctcactatggatatgaaccaatgagaaccctgnttactaaagggaaaatgctatggncaccggatggcttcnttnagtaataagtggccccnntntgggtaccatttggaaggnttaatgtaacccccaaccatcaanctttcttgcttnncttg

SEQ ID NO: 157 - DNA, 972 bp, Mus musculus
  misc_feature (328)..(966): n = any nucleotide
ttttttttttttttttttttttggtaaaagaacatgacaaaactttattttagctttttgggccaatgcttatccttatgttcagattacagaaaatactctttttaatatgcaaaaaaatcaaacatttctctaactgaaacatctaaaaatttggctcattgtcgaacacaaagtgacatatttggctttttaaaaaacacaacaactaattacagtgaagttgttacataaactatttttctcctaatgttcatggacagtctgcttctgtgtatatggcacatacactgaaatactcaagtgagacaagaaagatgatgtttgnggttacataagccatccaaatgtaaccagtgagtgcacttattactaaagaagccatccgtgaccatagcactatcccattagttaagcagtgttctcatttgatcatatccatagctgagaaataacacaaacctagaatgtggccaggtgctctgtaagtctgatgtgaatgtctaaaaagtatgacaggaaattcagaattctgctttccttcaggaaaacttcccagacggaggtggtgggccaagaaatccaccagcctgcttggtggcagctcgtgaaggggcgaggccaagaatcttctggatgttntgtcgaattgacatggtacagaggatatacaggaagatgaaggagcagtccgtggngcatctccccagcanggttcccgatgagaaagtccntggatgtaaagaacagangantgaaagggggagcntttgcccccacttttacccttcaaataatgggaattaaaccanttcccaattanggcggtaaaaaccaaaaggcccaattggggaaacatgggaatttcatcccgnanccatttggncagggcctnctaaatggggggncctttnaaantcccctttcttgggcccttntaaaacttncnttctnttgggggggccnacccaa

SEQ ID NO: 158 - DNA, 685 bp, Mus musculus
  misc_feature (12)..(680): n = any nucleotide
tttcgccccccntttggtaccgagctnggatcctntagtaacggccgccagtgtgctggaaaggcgacatggggaggcgcggaggcgacaccggaagtggctgtggtccgggtcggcccgagggggactcagcgcccgcagcaaccacccgcgcggcggcggtcagagctnanacccgtgcgggtgggcgtggcgggcgccgggacacagctccgactgttcgctgtcggcgcggggcggcgccgcgtcgctcgccttnaccagctgccatgagcgagcgcctccgccccagaaaaaggagaagaaatggcagtgatgatgacaaccaccctcctccccagaccaaaaggagcagtaggaaccccatcttccaggactcctgggacacagagtcttcaagcagcgacagtggtgggagcagcagcagcagcagcatnaacagcccagacagggccagngggccagagagcagcctgagccacaccatccccggatcctgccccagcaccccccagccgatgcctgagcagtctgcactatgccaaggcccttacttccacatnaaccagaccctgaaggaggctcactttcatagcctacagcaccgaggccggccgccgacatgatgctcctccggcagnttcttgccttctgtgaagggacagcactgtgcagattgnatatt

SEQ ID NO: 159 - DNA, 610 bp, Mus musculus
  misc_feature (8)..(610): n = any nucleotide
cgaacgcnagttctagcatttaggtgacactatagaatagggccctctagatgcatgctcgagcggccgccctttttttttttttttttctgtgtgcaacttttaaaaacaaatcattaagttgaaatatccaatctgcacagngctgtcccttcacagaaggcaagaaactgccggaggagcatcatgtcggcggccggcctcggngctgtaggctatgaaagtgagcctccttcagggtctggttgatgtggaagtaagggccttggcatagngcagactgctcaggcatcggctggggggngctggggcaggatccggggatggngnggctcaggctgctctctggcccactggccctgtctgggctgnngatgctgctgctgctgctgctcccaccactgtcgctgcttgaagactctgtgtcccaggagtcctggaagatggggttcctactgctccttttggtctggggagagggngggntgncatcatcactgccattncttctcctttntctggggcggaggcgctcgctcatggnagntgnngaaggcnancganncgnnnccgcccnnnccnncagnnaacagncnnngctgngnccgnn

SEQ ID NO: 160 - DNA, 684 bp, Mus musculus
  misc_feature (2)..(649): n = any nucleotide
ancgacccccccnttngaccgagctcggatcctctagtaacggccgccagtgtgctggaaaggctgattgctgaggtgggagtgggcccancgcccggnggccgcagctcacgcgcaacctgcgccatggccgcctccgccgcctcctccgagcatttcgagaagctgcacgagatcttccgcggcctccttgaagacttacaaggggtgccggagcggctgctggggaccgcggggacagaagagaagaagaagctggtcagagattttgatgaaaagcaacaggaagcaaatgaaacgttggcagagatggaggaagaactacgatatgcacccctgactttccgtaaccccatgatgtctaagctgcgaaactaccggaaggaccttgctaaactccaccgtgaggtgagaagtacacctctgacagccgcacctggaggccgaggagacctgaagtatggcacgtatgccttggagaacgagcatttgaatcgactacagtctcaaagagcattactcctacaaggcactgaaagcctgaaccgggctacccaaagcattgagcgttctcatcggattgccacagaaactgatcaaattggtacagaaatcatagaagagttgtnggagcaacgagaccagttgnaacgtactaagagcagactggtaaatacaaatgaa

SEQ ID NO: 161 - DNA, 585 bp, Mus musculus
tttttttttttttttttttttaagtttgaaggagtcataaacaacatttattaccttagtatatcatactggtcttgttgctgttccttcacattttcgaggttttccattgccctccctcccatagtctgacaagacaatacattcttgaactgttagcccacagcagcaacacatatccctatgtaaaaccattcattcagagtaagatgctggtccacaaggctttccctacagaagttcaatggtgtcgaaagaatttgtaatacaccagaccgaccaggatggctagctccagcaagatgatgacggagagcagcaacttgttggttatcacttttctggacattgagcgaagaatcttccggcttttgctcaaattttcatttgtatttaccagtctgctcttagtacgttccaactggtctcgttgctcccccaactcttctatgatttctgtaccaatttgatcagtttctgtggcaatccgatgagaacgctcaatgctttgggtagcccggttcaggctttcagtgccttgtaggagtaatgctctttgagactgtagtcgattcaaatgctcgt

SEQ ID NO: 162 - DNA, 662 bp, Mus musculus
  misc_feature (2)..(420): n = any nucleotide
tntgacgccgttctagcattnaggtgacactatagaatagggccctctagatgcatgctcgagcggccgcccttttttttttttttttttttccgttataatagccatctttatttgtaaaaatccagatataaaacgtaatctttcagtctttccaggttttccttttttacaaaaacaaaaaggcacgtataaaccttgcccgctgtcgtccccgtacacggngtttctcaggcagccctcccccccgccccgccccccgttacagctacatgcttcattccaggacgtctgcatccccacatgctttggngctttcctaccagggtagagttccgagctccaagacttgaagtacacaaagagggggtaggggtgggtgcagngtgtggcacaatgttccacggcgtgcagggcagngggctagtagtaggtctccttctccacccagccgccagggcgccgcctgataatgagcttccgcacctcgtcatacacaaagatgagaagggagtaggggaaggcacagaaccaccatgtaggtttgaggggatacatcctaaaggctgcccccatcccggggcagtaggataagaaagcagcaagggctgtctcttcaaagaggccaaatatcaagatcttggtcttcattccctgctgga

SEQ ID NO: 163 - DNA, 681 bp, Mus musculus
  misc_feature (6)..(656): n = any nucleotide
ttccanccccccttggaccgagctcggatcctctagtaacgtccgccagtgtgctggaaaggggttgccatggggattgttggctctgatgtgtccaagcaagctgctgacatgatccttctggatgacaactttgcctccattgtgactggagtagaggaaggtcgtctgatttttgataacttgaagaaatccatcgcttacaccctaaccagtaacattccggaaatcacccccttcttgatatttattattgcaaacattccactgcccctggggactgtgaccatcctctgcattgacttgggcactgacatggttcctgccatctccctggcctacgagcaagctgagagcgacatcatgaagaggcagcccagaaaccccaaaacggacaaacttgtgaacgagcgtctgatcagcatggcctatggacagatcggtatgatccaggccctgggaggcttcttcacttactttgngattctggctgagaacggtttcctgccctttcacctgttgggcatccgagagacctgggatgaccgctgggtcaacgatgtggaggacagctacgggcagcagtggacctacgagcagaggaagatcgtggagttcacctgccatacagcgttctttgtcagtattgtggtagngcagtgggccgacttggtcatctgc

SEQ ID NO: 164 - DNA, 683 bp, Mus musculus
  misc_feature (5)..(680): n = any nucleotide
ttcgncccncttggtaccgagctcggatccttagtaacggccgccagtgtgctggaaaggtctgcggccctcgcagaacttccagcagcgacatgttgggccagagtatccggaggttcacgacctccgtggtccgtcgcagccactatgaggagggtccggggaagaatttgccattttcagtggaaaacaagtggcggttgctggctatgatgaccgtgtactttggatctgggtttgccgcacctttctttatagtaagacaccagctacttaaaaaataaggatatttaattcatccctttaacagaatgaagaaagtttaagaggtgatctgaaaattggattaaactcttgaactcttatactagaaaaaattgtaaataaactaatgacataaagattcaaaaaaaaaaaaaaaaaaaaagggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggagagnagangcaggacagcaagggngac

SEQ ID NO: 165 - DNA, 587 bp, Mus musculus
  misc_feature (282)..(470): n = any nucleotide
ttttttttttttttttttttgaatctttatgtcattagtttatttacaattttttctagtataagagttcaagagtttaatccaattttcagatcacctcttaaactttcttcattctgttaaagggatgaattaaatatccttattttttaagtagctggtgtcttactataaagaaaggtgcggcaaacccagatccaaagtacacggtcatcatagccagcaaccgccacttgttttccactgaaaatggcaaattcttccccggaccctcctcatagnggctgcgacggaccacggaggtcgtgaacctccggatactctggcccaacatgtcgctgctggaagttctgcgagggccgcagacctttccagcacactggcggccgttactagtggatccgagctcggtaccaagcttgggtctccctatagngagtcgtattaatttcgataagccagtaagcagngggttctctagttagccagagagctctgcttatatagacctcccaccgtacacgcctaccgcccatttgcgtcaatggggcggagttgttacgacattttggaaagtcccgttgatt

SEQ ID NO: 166 - DNA, 684 bp, Mus musculus
  misc_feature (13)..(34): n = any nucleotide
ttccgcccccccntggtaccgagctcggatccantagtaacggccgccagtgtgctggaaagatgaattcaaagagtgcccagggtctggctggtcttcgaaaccttgggaacacgtgcttcatgaactcaattcttcagtgcctgagcaacacccgagagctgagagattactgcctccagaggctgtacatgcgggacctcggccacaccagcagcgctcacacggccctcatggaagagtttgcaaaactaatccagaccatatggacgtcgtcccccaatgatgtggtgagcccatctgagttcaagacccagatccagagatatgcgccacgcttcatgggctataatcagcaggatgctcaggaattccttcgtttccttctggatggtctccacaatgaggtgaaccgggtggcagcaaggcctaaggccagccctgagacccttgatcatctccctgatgaagaaaaggggcgacagatgtggaggaagtatctggaaagggaagacagtcggattggggatctcttcgttgggcagctgaagagctccctcacatgcaccgattgtggctactgctctacagtcttcgatcccttctgggatctctcgttgcccatcgcaaagagaggttaccctgaggtgacgttaatggattgtatgaggctcttcaccaaac

SEQ ID NO: 167 - DNA, 584 bp, Mus musculus
  misc_feature (235)..(584): n = any nucleotide
ttttttttttttttctgaattttttaaaaaaaacttaggacgtggggccaccacagggaagagggaagggccgcagctcctcaatgctacatacgggagggtggactggccagttcatagaagagcaaataggcgtcgctggtgcgcacttggctggaggacatgggtgtgacactggaatcattgaaagtgtgccattcgcctgtaaccggacttcggcagtaggctgtatagnggcctcccatggnggttccggagtgattggacacagcatacaggttgtaaacagcatggttggngttttctgaagcaaattctctcaagtccaggtctcttagngggaaattcacaaatgttgngagcttgctggttcgtatcctggattctgagaatcgcttcaggtggagcaccaagatctttgggaacctctggacagagaacttttttatgcatcgctttctggctcggcagcggcagcaagttggcttctcatcaccatccaatatgtcctctttggtgaagagcctcatacaatccattaacgtcacctcagggtaacctctctttgcgatgggcaacgagan

SEQ ID NO: 168 - DNA, 735 bp, Mus musculus
  misc_feature (20)..(732): n = any nucleotide
agggcgcctgagtgaaaagngnggcaccatggcctctgngctgtcctacgaaagtctggtacacgccgnggccggagccgngggaagngngactgccatgacagngttcttccccttggatactgctagacttcggcttcaggtcgatgagaaaagaaagtcaaaaacgacgcatgcagngctcctggagatanttaaggaagaaggcctcctggcaccataccgaggatggtttccagctatttccagtctctgctgctccaattttgtctatttttacacttttaatagcctcaaagcagngngggtcaaaggtcagcgttcttctacaggaaaagatctcgnggnngngttngtagcaggagnggtgaatgngctgctgacgactccgctctggnnggtaaacaccagactgaagctgcagggggcaaaatttcggaatgaagacnttataccaactaactanaaaggcattatcgntgcattccaccagattattcgngntgaaggnatcttgnctctgnggaatggcaccttcccctcccttgctgatgnacttcaaccctgccatccaattcatgntctatnnnggcttaaaacggcagcttctaccgaaacgnctgannctctctttctctgcntnngtccntcatttgncgcactanncaaagctnattgtcnccacagcnnccctatcccctncagancggtncctgtcnattctgaggcntncac

SEQ ID NO: 169 - DNA, 773 bp, Mus musculus
  misc_feature (172)..(758): n = any nucleotide
tttttttttttttttcagggtttcaatattttcatatcagtctaacctctacccccaaatacaaccaaacaccataaaaacaccaaaaaaccccataaaggtgacaggcttagctggctatactccctgcttagcctttgatgcctttacagccaatgactggaatgtctgnggngnggatgggaccaacgggaaaggagaactgttcctcttctccctgtgcagcacctgcgtctccacactgtcctgngggcgggcctcagngcttatgtgtgctcttcaggcccattacggngaaggtagcagctgtcagtttctcgtacacaaggaacatgagggcggctgtgagcactgtctgcagcagcttagcttccaggcctttgtagagtcccattattccaaagcgcttgactcgctggtgaagaagagagagaacattccgaagacttcccagggtcctgttttctgggttcagtctatgacgtccaaacctcagaattgactgtaccgtctgcatgggataggtgactgnggnggcaatcgctntggctattgcgccaatgatgaacacatncagagaagagagcttcatccgnttctttagaagctgccgttttaagccttcatagaacatgaattgnatggcagggttgaagaccaacagcaaggaggggaaggtgccattccacagagccaagatcccttcatctcgaataatctggtgnaatgcatcgataatgcctttgtagttagctggnataatggcttcaatt

SEQ ID NO: 170 - DNA, 656 bp, Mus musculus
  misc_feature (222)..(644): n = any nucleotide
agtagagcccctacggtcatggcggctaccgcccccaaggccgggggttcagctcctgaggcagcgggttctgccgaagctccgttgcagtacagccttcttcttcagtacctggagggcgacaagcgtcagccccggcttctggagcctggcagcctgggcgggatcccgagtcctgccaagagcgaggagcagaagatgatcgagagggcaatggaaagntgcgccttcaaggctgagctggcctgngtaggagggttngnctngggaggcgcgntngntatctttactgctggcattgataccaacgtaggctttgacccaaaggacccttaccggacaccaactgcaaaagaagtcctgaaagacatgggacagagaggaatgtcctatgccaaaaactttgccattgagggccgccatgttttcatgtactgagngnctggtagagtcttaccggggaaagtcggactggaagaacagcgtcatcagnggctgcatcactgncggagccatcggcttccgagctgnagtaaaggccggggccataggttgnggagggnatgctgctttcnctgctgcnatcgnttnttacctacgnngaaggaaanagcctnctaaggaaagaggacnccagccncttnagagctgctctg

SEQ ID NO: 171 - DNA, 755 bp, Mus musculus
  misc_feature (118)..(752): n = any nucleotide
tttttttttttttttttttttttttttttttttttgcctcgatggcataagtttactttgccctgccctgggactcatgatggatacttgcacacagaaggaagaagagcttcccggntatgggcaaacactgccacttccctattcaaagctggggagctgagccaggtcagaactgagcctggttcaaagcttaangcgtaacagtccacacactgcctcctgatctgtttactgcaaggagacaatntgccataccacctnccctgaaaaacctggttggaattcaagtggaaaagtatggttgttccaanggctgcagagagcgatcgagcagcgaggngngtccttnacaccatnaactgtagagggatgcacttggcttcacccctgaagcagtccaagtgtgtagaaactgntccacagagcagctntgaaggnggctggcgtcctctntccttcccggctgnttccttcaccgtaggtaataatcgattgcagcagagaaagcagnaaaccntncacaacctatggccccggcctttactccagctnggaagccgatggctccgccagtgatgnagccactgatgacgctgntcttccagncccgactttccccggtaagactntaccagacncttagtacatgaaaacatggcgcccacaatggcaaagtttttggcataggacattcctctctgncccatgnctttcnggacttcttttgcagatgntgcccggaaagggcnctt

SEQ ID NO: 172 - DNA, 664 bp, Mus musculus
  misc_feature (33)..(660): n = any nucleotide
aaggccgggaggatgcggccggagcccggaggntgctgctgccgccgcccgatgcggncgaacggctgcgtnaagaacggngaagngaggaacggntacttgaggagcagcaccgccaccgncgcggctgccggccagattcatcatgtaacanaaaatggaggactgtacaaaagaccgtttaatgaagcttttgaagaaacacccatgctgncngctgngctcacatatgnnggctatggcgtactcaccctctttggatatcttcgagatttcttgaggcattgnagaattgaaaagngccaccatgcaacagaaaganaanaacaaaaggactttgcgnccttgtatcangnttttgaaancttctatncaaggaacctctacatgagaatcanagacaactggantcgacctatctgtantgngcctgnagccaaggtggatatcatggngagaaaatctnatgactataactggtcattcaagtacanannnaatatnnttanaggtgtcataaacatggnttcctacacctatcttgtntttgcgaggnacactnnatcatntcangnngcccctnctgaantcctcacngagtattncagcagccctgngcaancnctcgcannnnantnctaacctntacangcatncctnaact

SEQ ID NO: 173 - DNA, 778 bp, Mus musculus
  misc_feature (33)..(765): n = any nucleotide
ttttttttttttttttttttttagcagtttggnttttttactatttacaaaatgccatttggagtgaaggtggccaccttcagtagcttcagaaatgtctttcacatgaagnggtcactgaagngnggttcctggaattggctcagaaaggccacacactgtcctgggaggaattatccccctctagggagcaccagaaaggctcagtcttctgtctcttcataggtagtctcatcaaagggcctgtccagtagaggcaccagccggnggcgagagtactttagctgcagcagatccccaacttcatctatctccttcaaagcagngtcaagtatttctttggtatgagctgctgacaggcaaaatctggctctggactcaatgatcggggtagcaggaaatcccaccacaactacaccaatgttccgcttcagcatctctcttccaaaggcgccaattttggccggcatgtagagcatcaaaggcaccaccggggagtcttcattgccatagatgatgaaccccatttccttcaggcgtctcctgaaatacctggngttctcagccaactgctgtatacattctttgccaagactggagccatcctgccccatgatgnacttcatngaggtgataatctgttccatcacaggcggtgacatcggaccgtggcatacacagcactgtgngaatgtntgcgcaggtagnctatcngctccttcttcctcccnatgnatccctcttgaangcaccnnagctnttttntgaatgttcccat

SEQ ID NO: 174 - DNA, 779 bp, Mus musculus
  misc_feature (69)..(706): n = any nucleotide
agcggatcttcgggccgggaggacattcggcctctgtgagccgcaaccttgcccagcgagcggttgggngttcgccatcttaggaggatgttctcgtccgtagcgcacctggcgcgggcgaaccccttcaacgcgccacacctgcagctggtgcacgatggcctgtcgggtccccgcagtcccccagctccgccccggcgttcccgccacctggccgccgccgccgnggaagagtacagttgngaatttggctccatgaagtattatgcactgagnggctttggnggggtcttaagttgagggctgacacacactgctgnngttcccctggacttagtaaagngccgcatgcaggnggaccctcagaagtacaaaggcatatttaatggattctccattacactgaaagaagatggcgttcgnggttnggctaaaggatgggccccaactttgatnggctattccatgcaagggctctgcaaattcggcttttatgaagtcttcaaagccttatatagcaacatacttggtgaggaaaacacctacctgtggcgcacatcactgtatttagcttcttctgccagagctgaattcttcgctgacattgccctggctcctatggaagctgctaaagttcgaattnaaacccagcctgggtatgccaacaccttgaggnaagctgttcncaaaatgtataaagaggaangcttaaatgcgtctacaagggcgatgctcctctgtggatgagacagatcccctacaccatgatgaagttcgct

SEQ ID NO: 175 - DNA, 754 bp, Mus musculus
  misc_feature (296)..(754): n = any nucleotide
tttttttttttttttcaggaatagatgggtttatttagcattaagtttgaaactcttgctttaagcaagggtactacagtaattatatcaggaacagacatttcctacactgtcaaatatataaaagttcctttgcactttcaacactgatcaacaagcagattcagtccatgttactttgatgtatctactcagttaacccaagcttcttcttcagagactctggcatctcagggggaggagggcgagggagcctgaagtagaccttcacagagtcgtagatgaaccactgtagngcagtcagagtgccaatcatgatgattcgggcgaagagccccttccacacacctctgaagcccagcctctgcaggacctgagacgcggagctgcctttctctttattcagcacagagaccacagagtcagcagggngggagacgatcgcacagaagactccagctatgtaacctgccacaaatgtcacaaccagctgctctgcctttgtacattcacttcggggcttgggaaccacaaatttgtacaaagcttcaacagtacgttcaaagcaggcgaacttcatcatggngtacgggatctgtctcatccacagaggagcaacgcccttgtagaacgcatttaagccttcctctttatcattttgggaacagcttccctcaaggtgttggcataaccaggctgggttttgaattcnnaactttagcagcttncataggagccagggcaatgtcagcgn

SEQ ID NO: 176 - DNA, 826 bp, Mus musculus
  misc_feature (84)..(811): n = any nucleotide
aggcactgctggcgacatggccgacacggacccgggctatccccgctcgtccatcgaggatgacttcaactacggcagctgcgnggcgtcggccagcgngcacatccgcatggcctttctcagaaaagtctacagtatcctctctctgcaagtcctcctgactacagngacctctgccctgttcctgtatttccaagctctgcggacatttgtccatgaaagccctgccttaattgaggagntagctctgggatctctgggctngatctttgcactgactctgcacagacacacgcatcctctgaacctctatctactctttgcatttacactgtcagaatccctggccgaggcagctgnngataccttctatgatgtatatctggttctgcaagcgtttataatgactactgcagtctttcttggcttgactgcctatactctacaatcaaagagagatttcaccaaattcggagcagggtngatagctggnnatgaggatnntgagcttggcaggattcttgaagctgantttttacagagagacgatggagctggncttggcctctctaggcgccctcctcttctgtgggntcatcatctatgatacacactcgctgatgcacagactctctcccgaagagtacgtgaacgctgncatcagtctctacatggatatcatcaacctcttnctgcacctggtgaagtttctggaagcagntaaataaaaagtaaccgagcagtngttcanagacaggtctattatgaaaggangctttggaattnaactttaaatggttaataattaaangccaaatgtgaactt

SEQ ID NO: 177 - DNA, 775 bp, Mus musculus
  misc_feature (105)..(661): n = any nucleotide
ttttttttttttttttttttaaagatcacatatgcttttaatattaacatttaagtttaattcaagcgtctttcataatagaactgtctctgaacactgctcggntactttttattaactgcttccagaaacttcaacaggtgcaggaagaggttgatgatatccatgtagagactgatggcagcgatcacgtactcttcgggagagagtctgtgcatcagcgagtgtgtatcatagatgatgaacccacagaagaggagggcgcctagagaggccaagaccagctccatcgtctcactgtaaaaaaacagcttnaagaatcctgccaagcacaaaatccacaaaccagnaaacaaccctgctccgaatttggtgaaatctctctttgattgtagagtataggcagtcaagccaagaaagactgcagtagtcattataaacgcttgcagaaccagatatacntcatagaaggtaacaacagctgccacggccagggattctgacagtgtaaatgcaaagagtagatagaggttcagaggatgcgtgtgtctgtgcagagtcagngcaaagatcaagcccagagatcccagagcaaacaccacanttaaggcagggctttcatggacanatgtccgcagagcttggaaatacaggaacaggncagaggtcactgtagtcaggaggacttgcagagagaggatactgtagacttttctgagaaaggccatgcggatgtgcacgctggccgacgccacgcagctgccgtagttgaaga

SEQ ID NO: 178 - DNA, 803 bp, Mus musculus
  misc_feature (524)..(791): n = any nucleotide
agaagaaaaggccgaaaaggccaaaattaataaggccattcagaagggcaacatggaagttgcgaggattcacgccgaaaatgccatccgccagaagaatcaaggggtgaacttcttgagaatgagtgcacgagtggatgcggtggcggcccgtgtccagactgcagtgacgatgggcaaagtgaccaagtccatggcgggtgtggttaagtcgatggacgcgacgttgaaaagtatgaatctggagaagatctccgctttgatggacaaattcgaacaccagttcgagactctggacgtccagacgcagcaaatggaagagacaatgagcagcacgacgacgctgaccactccccagaaccaggtggatatgctgctccaggaaatggcagatgaggcgggcctcgatctcaacatggagctgcctcagggccagaccggttccgtgggaacgagcgtggcttcggctgagcaagatgaactgtcccagagactggcccgccttcgggatcaagtctgacggncgaaccagtccgagatttccctttgacgtgctctctgggttttagagagatgtccnagaattgtgccagaatgccgaacggtcttctaagaacctacagccctnaccagatangctgagaaattccagttttctccacttctaactggattttaaagctctttctagcttatgatgatgtgtattttttatagctgccttttaacagaactggttcattnctctacataaatctaggaaaaaatcgacngactntagccctcatgnnttccgtttttac

SEQ ID NO: 179 - DNA, 815 bp, Mus musculus
aagagtatttcccaccgaagaagttgaaccaagtgtttacatacttgttagtacccattccttctcctttcattttaatgcaccgcttgccagtctctgtaccatcaaccacactaaaaataacttcagtctgataattctaagtaaaaacggaatacatgagggctagagttctgtcgattttttcctagatttatgtagagcaaatgaaccagttctgttaaaaggcagctataaaaatacacatcatcataagctagaaagagctttaaaatccagttagaagtggagaaaaactggaatttctcagcgttatctggtgagggctgtaggttcttagaagaccgttcggcattctggcacaattctgggacatctctctaaaacacagagagcacgtcaaagggaaatctcggactggttcggccgtcagacttgatcccgaaggcgggccagtctctgggacagttcatcttgctcagccgaagccacgctcgttcccacggaaccggtctggccctgaggcagctccatgttgagatcgaggcccgcctcatctgccatttcctggagcagcatatccacctggttctggggagtggtcagcgtcgtcgtgctgctcattgtgtcttccatttgctgcgtctggacgtccagagtctcgaactggtgttcgaatttgtccatcaaagcggagatcttctccagattcatacttttcaacgtcgcgtccatcgacttacaccacacccgccatggacttggtcactttgcccatcgtcactgcagtctggacacgggccgccaccgcatcc

SEQ ID NO: 180 - DNA, 397 bp, Mus musculus
  misc_feature (16)..(383): n = any nucleotide
agcggacgcgcccagnagccgngctgcagagagntncatcgngcgaccgctgccgcaggcgcttgctccgagtagccctgcggctccgcttctgccatgangatccacggctttcagagcagccaccaggacttctccttcgggccttggaagctgacggcgtccaagaccnacatcatgaagtctgcgnatgtggaaaagttagctgacgagctgnacatgccatccctccctgaaatgatgttnggagacaacgttctaaggatccagcatggctctggctttgnaatagagttcaatgctacggacgcactgagatgngtgaacaactatcagngcangctcaaagtagcttgngctgaagagaggcagnaaagtagganggagggcgaacact

SEQ ID NO: 181 - DNA, 686 bp, Mus musculus
  misc_feature (60)..(686): n = any nucleotide
tttttttttttttttaaaatggcatatgtcatatttagaaatggccaaggacacacctgnntactcntcacagggatacactaatacaatcttcagatgcacactcctcctccccaccgnccacctctttaattttttttaaaaagncnnttattttattttaattctccacaatttttaacaatgaattatcaanataagnaatctaacantactcagagacaaagacatttaaatgaaaagatttaaaaantacttgtgtgtacanacacacacactntgttgcccagaaactccaactaatgactaatacaagattaattttgtaggtnacatgtnacanctctccgtaagtgcgaacaaacaccanaaagcccacagccaagagccagagtagcacatnagcatgcagtcagcatcctatggcgnccagcaggtcccaggcatcggngctcagtgacagccttacagtcagcctcctcacagtgaagacgatgcttnaggccggntgcagaccatgtcccctcatgagacccanacacatcactgctgacgtaagctccagagcacatctctctctgnagcaggaacttgaaaacagacaggataaanaggagttgtgtggaacctgcctgcagcaatcttttattntccnttanggctacagnaanatctgcgcatangcn

SEQ ID NO: 182 - protein, 272 aa, Mus musculus
MMIHGFQSSHRDFCFGPWKLTASKTHIMKSADVEKLADELHMPSLPEMMFGDNVLRIQHGSGFGIEFNATDALRCVNNYQGMLKVACAEEWQESTREGEHSKEVIKPYDWTYTTDYKGTLLGESLKLKVVPTTDHIDTEKLKAREQIKFFEEVLLFEDELHDHGVSSLSVKIRVMPSSFFLLLRFFLRIDGVLIRMNDTRLYHEADKTYMLREYTSRESKISSLMHVPPSLFTEPNEISEYLPIKEAVCEKLIFPERIDPNPADSQKSTQVE

SEQ ID NO: 183 - DNA, 288 bp, Mus musculus
  misc_feature (71)..(283): n = any nucleotide
gggccgcagcaggcgcgagcgcccaggacctgccagctgagccttccgccgtcgccatgggacagaacganctgangggcacggccgaggacttcgccgacnanttccttcgagtcacnaagcagtacctgcctcatgtggcgcgcctctgcctgatcagcaccttnctggaggatggcatccgcatgtggntccagaggagngagcagtgtgactatatcgacaccacctggngctgtgnctacctgntggcctcatccttcgcgtacctcaacctgctgcnacaat

SEQ ID NO: 184 - DNA, 496 bp, Mus musculus
  misc_feature (11)..(496): n = any nucleotide
ttttttttttntttttccaaagattttatctccaattttcaggtgtctgcaaataaagttctcaaaacatctgngcctttaccaaggnaggggaagggagaaggacacacaaacgccggcagnttgntgactttgccctgaaccaggccagggccctgcgcctcanccagggaggnnaggnatcagngngttaccactcttttttcttctcatncatggacacacccnctgntccaagagccaccatcaggagcaggcctccaatcactgacatggtctgnaagaagtcgtatttcaggaagtnntgcntgnatttatacaccgggatngactcananggcgtngaannacacnctgntgncaanncagccngnntactgngagncncnagntgccngcntnnctctacaantnnanggnnactatcnncnncncnnnntncccccctnnctnnntctnnncnccnnnctnttcnttctancncaccatnnnnccn

SEQ ID NO: 185 - DNA, 514 bp, Mus musculus
  misc_feature (12)..(491): n = any nucleotide
agcttcttcagncggnacggggtcagcgagcggntgcttcgtggagcagagaggtgcatnacnaggttcccgatgaacccagagaacnctccaccgtatccgggccccgggccaacagccccatacccaccttatccanaacagccaaaggggccaatggggcctatgggagccccacctcctcaggggtacccctacccaccanctcaggggtacccctatcaaggatacccanagtacggctggcagggnggacctcaggagcctcctaagaccacagngtatgtggnggaagaccaaagaagagacgacctgggcccatccacctgcctcacagcctgctgnactgctctgtgnngctgctgcctctgggacatgctcacctgatcanctgatgagcccagctcttccgcttggccgctctgtgccacctccgataagngtgccnggccccatctcttctgatngctataaagnggctagctctgcgnagacacctctactttctgtccta

SEQ ID NO: 186 - DNA, 584 bp, Mus musculus
  misc_feature (70)..(566): n = any nucleotide
ttttttttttttttttttgattttggacagatttattgaaacataaagggtatgagcagagagatctagnagngtgtcacatattgccattaccttgagtgtataatttaaacattataaatatatatttcataactaagcctttggccaaaaaagtaaattatttagcacattttttaaagatcagtaagaaatgagttttgaacattaaaaagatcaagtcactgaactaaatagcagtaaccctcactaatctaaaactccataggacagaaagtagaggtgtctgcgcagagctagccactttatagcaatcagaagagatggggccaggcacacttatcggaggtggcacagagcggccaagcggaagagctgggctcatcaggngatcaggtgagcatgtcccagaggcagnagcaacacagagcagtccagcaggctgtgaggcaggtgnatgggcccaggncgcctcttctttgntcttccaccacatacactgcggtcttaggaggctcctgaggnccaccctgccagccgnactgaggntatccttganaggggtnccccctgaggtggtggaa

SEQ ID NO: 187 - DNA, 1359 bp, Mus musculus
ttttatactaaaaaatgtgagtcccaaattccgcatttgtacaacaacgagggggagcgctcagacccctaccctaaacgggaccgcacccccaccgaacgagaagaggagcttgcgctccatccctgagggtcaacttatggccagttcaccgtaaccgctgcagatacctccagagtagtcggattttggacagatttattgaaacataaagggtatgagcagagagatctagtagtgtgtcacatattgccattaccttgagtgtataatttaaacattataaatatatatttcataactaagcctttggccaaaaaagtaaattatttagcacattttttaaagatcagtaagaaatgagttttgaacattaaaaagatcaagtcactgaactaaatagcagtaaccctcactaatctaaaactccataggacagaaagtagaggtgtctgcgcagagctagccactttatagcaatcagaagagatggggccaggcacacttatcggaggtggcacagagcggccaagcggaagagctgggctcatcaggtgatcaggtgagcatgtcccagaggcagcagcaacacagagcagtccagcaggctgtgaggcaggtggatgggcccaggtcgtctcttctttggtcttccaccacatacactgtggtcttaggaggctcctgaggtccaccctgccagccgtactgtgggtatccttgataggggtacccctgaggtggtgggtaggggtacccctgaggaggtggggctcccataggccccattggccccattggctgttgtggataaggtgggtatggggctgttggcccggggcccggatacggtggagggttctctgggttcatcgggaacctggtgatgcacctctctgctccacgaagcagccgctcgctgaccccgtcccgactgaagaagactggaccggactacgccaacagcgccgcacctgtcgcgagccactagagccgcagcggcgcgcctgacgtcacccgagcctcctgcgctggaagcccggagtcacccgatctccctggcaacgcgcgagggggcggcaccaagcgagggcggtgtcatagcgcgagcccgcccccaaaactctgcatcacgttggggacttaagcctgtcccttagctccttgtggtccccgatctgtatctatagtttcactccgggtccttgcgagcacccggccccacgtcgctaggaccggagattgggaaaaagggtaggggggctgaggaccagcttggtacacatcatgtagggctgctgctgctgctgctgcctgacctcttctccgctgctaggttcgggccgaggcctgttccga

SEQ ID NO: 188 - protein, 104 aa, Mus musculus
MNPENPPPYPGPGPTAPYPPYPQQPMGPMGPMGAPPPQGYPYPPPQGYPYQGYPQYGWQGGPQEPPKTTVYVVEDQRRDDLGPSTCLTACWTATCCCCLWDMLT

SEQ ID NO: 189 - DNA, 855 bp, Mus musculus
gtcaggcgcccggttgcattccgaacaggcaatctgagacaggtgcggcaagtctactgcgggctggtccgggctcctcaggttcagacccgaccgttatccagtcggttcgtggagaggagaggtgcactttacaggtccccgatgaaccaagagaaccctccaccatatccaggccctggtccaacggccccatacccaccttatccaccacaaccaatgggtccaggacctatggggggaccctacccacctcctcaagggtacccctaccaaggatacccacagtacggctggcagggtggacctcaggagcctcctaaaaccacagtgtatgtggtagaagaccaaagaagagatgagctaggaccatccacctgcctcacagcctgctggacggctctctgttgctgctgtctctgggacatgctcacctgaccagaccagcccagccgtcctgtcctgccagctctgctgccacctctgacaggtgtgcctgcccccatctcttctgattgctgttaacaaatgactagctttgcacagacacctctaccttcagcactatgggattctagattaatgggggttgctactgtttaattcagtgacttgatctttttaatgtccaaaatccatttcttattgatctttaaagatgtgctaaatgacttttttggccaaaggcttagttgtgaaaaatataatttttaaattatacattcaaggtagtggccaaatgtaacacatcaatcatggaatgatttctctgctaacagccgcctgtatgtttcaataaatttgtccaaagctcaaaaaaaaaaaaaaaaaaaaaaaaaaaaaaaaaaaaaaaaa

SEQ ID NO: 190 - protein, 97 aa, Mus musculus
MNQENPPPYPGPGPTAPYPPYPPQPMGPGPMGGPYPPPQGYPYQGYPQYGWQGGPQEPPKTTVYVVEDQRRDELGPSTCLTACWTALCCCCLWDMLT

SEQ ID NO: 191 - DNA, 233 bp, Mus musculus
  misc_feature (2)..(233): n = any nucleotide
gngtggccctgcctcgctggggcctgacctggtntcccaacctggagnccagaaggnggctttctgcggagccgnggaggaaggacgtgntctgcgacggantctagcaggcagggganttgagctgggcctggccctgggcacagagtcacngnagctatnganggactccgcagatgnggactcagctgaggagggngnngtggangccgncanctnagacncnancctcn

SEQ ID NO: 192 - DNA, 748 bp, Mus musculus
  misc_feature (33)..(746): n = any nucleotide
tttttttttttttaaagtcactcagtttattanaagccatgggaaatctgagagaaacgttccaagcacttgntgctcctgagcatcaaagatggagtgggnggcttcggcatcagtcccccattggcagccacagggcctttgntcgngctgcgtgaagactcanntcctgtcctgccccctggacctccacatcccaggcccttgggaagcccctgtttaccnacacagacttgaggtttcctgcagaggcccattccacaaactgggagccctgggccgagccgaagtaccacagggcttggacatcctgatgtaaggccagacagcggntcaggnggncgcgatcccctgtcactacactcaccaggccagcaggaaacagaggagctatatcctggcagacctccaaggccagcagaggacatgccccactgggtactaagaccacggcattgccatggnccagtgcaggggccagtagtgacacaaaagccagcaggggccactcatccgggcacacnanggncagcactcccaatggttctcgaagccgnagcacagggcctctnaatcctgttacctgaagtgtctggccttggtcctgaacccgntgccccatgtntgnagtcgccttanactcagtttacttcaatcttggcnactttaggcgctgtcccgtgccttctagttgtgaggnnagcactggcttcctgcnttcagagcagccnnngngcccaagnnnnct

SEQ ID NO: 193 - DNA, 483 bp, Mus musculus
  misc_feature (12)..(469): n = any nucleotide
agatggagcagngtttccaacanggacgacatnaagacctcactcaagaaagttgtgaaggagacatcgtatgagatgatgatgcagtgtgtatcgcgaatgctggcccatcccttacacgtgatctcgatgcgatgcatggngcagnttgnggnacgggaggccaagtacagaggngtgctgagttctatngggaagatcttcaaggaagaggggctgctgggattcttcgntggcttaatccctcacctcctgggcgangaggntttcttgnggngctgtaacctgctggcccacttcatnaatgcctacttggtggacgacagcgtgagtgacaccccagggggnctggnaaacgaccagaatcnaggttcccagtttagccaggccctggccatccggagctacaccaagtttgcgatggngatngcagcgagcatgctgacctaccccctttctgctcgctgnngatctcatggcagt

SEQ ID NO: 194 - DNA, 608 bp, Mus musculus
  misc_feature (34)..(608): n = any nucleotide
ttttttttttttttaccaccaatacatttattcnnggagatgggtctatcttaccacgaggggaggactagatgtcgntctatgnaacctgtgcgtattcgcacccagcacagtgactgaaccctcacacctggcgtcaccagcacagacaagcagatnaggnnatggtctgaggagaacatgatttcctattcaggagaaggcaccacccttgtataagaaaattagtgttgngaacatagcgccagnctcccatggcccaggtgtgatggcgcccaatttacaaagcaggaagtgggnngcggcggtgcttctggctgactggcaggatgagctgngctagaggngcagggaagccttgccactgagtgacgtttgcctctgcagcctgcctctgcctgagtacaagatggactccagtacctctaggcagnaagggnatgccaccccaccactgntcccccaggcttccccaggtcccaggtgacctacntccacnagcccanatntgnacagacncatnnccaaatatggacatgaagccagcntggncttgnagtcacccttgcncatctganatgatgggcccttcccngngttagngctan

SEQ ID NO: 195 - DNA, 762 bp, Mus musculus
  misc_feature (183)..(754): n = any nucleotide
agggcggaggaagcggactgttccggagctctgcctagccgggcccaacctttgctccagagatcatggctgccgaggatgtggtggcgactggcgccgaccctgagcgagctagagggcggcgggctgctgcacgagattttcacgtcctcctctcaaccctgctcctcctgggcctctgcntcttcctgctctacaagatcgttccgcggggaccagcccggtgctagtggcgacaacgacgatgangaaccaccccgctgccccgcctcaagnggcgcgacttcacccctgccgagctgaggcgtttngatggngtcaggacccgcgcnttctcatggccatcaacggtaaggtgttcgacgtgaccaaaggccgcangttctacgggcctgaggggccntatggggtctttgccggaagagatgcatcaggggccttgcncattttgcctggananagaagcactgaaggatgantntgacgacctttctgncctcaccctgcacagcaggagancctgantgactggnactctcanttcactttcnagtntcntcacntgggaaaactgctgaaggaaggggaggagcctactgtgtactcagatgatgaagancanaagatgagacagcttggangantgantgnnncattcggtggagcntatctatntttgtattttgcancnntcnttttgtancnttcnngtctgcnttncnacatggngatttnnntattanacagttttgcncttgctga

SEQ ID NO: 196 - DNA, 822 bp, Mus musculus
  misc_feature (233)..(795): n = any nucleotide
agctcgaaagcgacatggcggttctcttaaagctgggcgttctctgcagtggccaaggagctcgagctctcctactccgaagccgggtggtcagacccgcttatgtgtcagcatttctccaggaccagcctacccaaggacggtgtggtacccagcacattcacctgtcaccaagccaccactctggttccaaggctgcatctctccactggaccagtgagagggttgtcagngttctgctcttggggctgatccctgctgggtacttgaatccctgctctgnggnggactactctctggctgcagccctcaccctgcacagtcactggggccttggacaagtggttaccgactacgttcatggggacaccctgccgaaggctgccagggcaggcctcttggcactctcagctttgacctttgctgggctttgctacttcaattaccacgatgtcggcatctgcagagcggntgccatgctgtggaagctctgacctgggtgcagcactttgattgtgtgcctccttgcctctgctttaccaatgccgttcacctcgcagtgaggggggatgaaggataagcccattggtgggcagaatgtcttctaattacatggttattttcagaatttatttgttgaggaagaggtttgaggagttaggttcgaccattcgtgagtctgtgttccatactccactgagtgtgggcactagctcacagcctcgcggtgagactgaacatttcatgagctcatggtgcctttgaccaccattttcttaaggagagccagctgantgctgtcaggataagagcatctcttca

SEQ ID NO: 197 - DNA, 227 bp, Mus musculus
  misc_feature (33)..(226): n = any nucleotide
agctaggatctttagcttcaactcctactgctncttctaacccagcagccccggataatgcagcccaggaggagctcatgatcaccctgatcacaggattggcgtccctcacgtcgagaacctccatgggcatcatcggtggngggggccggtaatttngaaaacaggtgggnttgnaacctaatctttgcnncttaaggaatgaccggggcttngacctttaatna

SEQ ID NO: 198 - DNA, 789 bp, Mus musculus
  misc_feature (11)..(783): n = any nucleotide
agctgcgcccntgtaccctaggtctagagtgnacccgccccgggaaagaagctaggccgggtccgcagcatcagccatcacttcgcacaaagcagcgcagctccgggaccgccgaggaccacagcggcgcggcagcggcgcggcgacactcagtgcaccgtatgcccctgcgcccctgccgaggcaaccggagcgccccgagagaccgcgccgccgcggggtccaggtgcagttagcgagcctagcccgcagcgcgcagtcgcgggagagcggggagcggcaagcaacagggagcgggacggcggcgaggcgctcgcgggcccctcctgctgcccgcgcccggcgagctcatggcggccatccgcaagaagctggnggnggngggcgacggcgcgtgcggcaagacgtgcctgctgatcgtgttcagtaaagacgaattccccgaggtgtacgtgcccaccgtgttcgagaactatgtggcggacatcgaggtggacggcaagcaggtggagctggcgctgngggacacggcaggccaggaggactacgatcgtttacggccgctctcctatccggacaccgacgtcatccttatgtgcttctcggtggacagcccggactctctcgagaacatccccgagaagtgggtgcccgaggtaaagcacttctgccccaatgtgcccatcatcctggtggccaacaaaaaagacctgntcagcgacnagcatgtccgacggagctggcccgcatgaagcaggagccagtncngcacggattgacggncgctcc

SEQ ID NO: 199 - DNA, 791 bp, Mus musculus
  misc_feature (32)..(773): n = any nucleotide
aggtttcctcgccgccggccaagatgaaccgnttcttcggaaaagcgaaacccaaggctccgccacctagcttgacggactgcattgggacggnggatagcagggcagaatccattgacaaaaagatttcccggctggatgctgaactagtgaaatataaggatcaaatcaagaagatgagagagggtcctgctaagaacatggtcaaacagaaagccctgagagttttaaagcaaaagcggatgtatgagcaacagcgagacaacctggcccaacagtcctttaacatggagcaagctaattacaccatccagtcactaaaggacaccaagaccacggttgatgccatgaagttgggagtaaaggaaatgaagaaggcatataaggaagtaaaaattgaccagattgaggacttacaagaccagctggaggatatgatggaagatgcaaatgagatccaggaagccctgggccgcagctacggcaccccagagttagatgaggacgacctggaagcagagttagatgcgctgggcgatgagcttctggctgatgaagatagctcctacttggatgaggcagcttccgctcctgcaattccggaaggtgttcccactgacacaaaaaacaaggatggccgtgctgnnggatgaatttgnactgccgcagattcccgcttcgtagacttacaacattcagcacgtgatgtgaaacaacangagaagtattctggnactagnaaatagttccccgatctgccaaccagattaggnttctttcctttctttgat

SEQ ID NO: 200 - DNA, 752 bp, Mus musculus
  misc_feature (51)..(744): n = any nucleotide
ttttttttttttttttttttttttttttttttttccttttgaaaaatattnattgagtataaanaaatgncctatncattaaatatcaagaaatattcattattcattacaagtcaggctagtatactgagggaaccatggaatcaaaatagtanctcatgataccgaagagacagttcattccaacagtanatcgtgnaatgacttgctcacatgacacaacaaaatntatcaaaagaggccatgacttaaacccatntacaataccacaacagacaagggttctgaagcatactatcagcagtcccaatctcatcaatcacttttatcttatcaatgtatcagcncactgagactgtacttacnctaccatagaattcagagtctcatctccttggtttaatttcagattattgttaatgaatgaaaacaagtcatacaaagtcaagggtacttttcaattagtctttcctaagagaaaaaaaaaaaaangaatcaagttttagtaaagacagtttcccaaaagcaatgaattccttancggaaaaaataaaaatncnggagcagtgtananagctttttcttcaaagaaaggaaagaaacctaaatctggttggcagatcgggaactatttcctagtcccagaatacttctcttgntgtttcacatnacgggctggaatgttntanggtttacgaagcgggaaatttgnggnagnccaaattcatcccccagcacgccatncttgttta

SEQ ID NO: 201 - DNA, 1026 bp, Mus musculus
  misc_feature (4)..(1026): n = any nucleotide
aaanctnnatctcatctacacnngcnntngatccactannaacgggncgccagagacgctgnaaaggtccgctcctncgctgnagagnacgtgccctganctcnnnngacaaggcttccnttntanccgncancntttannantccntcntantnctcncctncnnnattnttatntnntactcncnnnctnctccctctntncntntctctncntnnncctactcttcctntnnccccttccctnnttatnnntctttatccactccntctcnnantatntnntnntnncaaatctatntctntnnccnnnncncccannnntctnnnaccntncctcntttcnntttncctactccctanttcctcntncttaccnccnnccattntcaatcnnnctnctntccaacnctcancnntatctttccccnntctntnancnatncctactcnntattcnccnccttctnccncnactanctcntcatttnncnnnaanccncantnntnntntnncnnnacnntntnatttcntacantncccnacnntcctnttnnnccactnntttctctannntactcttacnnaanntctnannctcccncnnnncntctntctccnttcanatcttncnnnccnnatnnacctntcatcncnctntccttnncnanannanntccncnttcntctttcntcactancttannccntnttntcnctcntattnntttanttntttcctcntntntcnnnntctntnnnctatcacctcttcctcnanttcnacncntntcttncnncatctnnntaantnttctnanactctcctattancctnccaactcccgtcctcnttctcccccncntncnntcnccntcnactccttcaatnatataatccctncacccnnnnctntanncctcntttatntcttcttaccacnaatcaantnaattcttnttcnatnctnncncncctttcaacncngctatttcacaacnnncacncnttcnnntnctacnctn

SEQ ID NO: 202 - DNA, 1353 bp, Mus musculus
  misc_feature (5)..(1347): n = any nucleotide
ttatnaaccnctntcnccacgcttggacccactancaacggncgccagtgtgcntggaaagnacgaanggcttatgcgcnaggtnccacncnnnttgggngncnaannngncntncnagaggnagggccnnttttnnnncggngnncnnannnnaagncgaangngtcnncntancncnnttnnncncccacgcccnnnncncnntnnanncccnatncnncannannctcnctctnttttnctccccncncctncttcnnctncnttatcnncnnctttcncctctctnncntacnctacccttccttcntnnncncccccnnnactcnccnccanncactnnnccantccttcntnnacctatcccccctccccnncctcacnctacantctcctccctntnccnnccctttcttcttccancctcttnctccnccntnnccccctcctcctctcntntattcatnntacnnttcattcnncnattcntttncnnacctnnnttctnncntccacannncttatncccatctcttatnnnacttctncnantctcctnntctcctctacntnnanctctcnnnntacnctntntctanacatctctctctcactntcnctcacnctatctctcctcnantnctcantnatantctcccactcctanncnctctcccccctctcantagttanctctntctccttcnctcntatagcntatcncaannctctccntacnctcncctcgtcatctatcancctctcacctntntcanactntcatanatcnctcctctnctcccatnatnttccacctcactnacnttntncntnntncttcntatcctccnatccnttccntncanncctctctccctctcaaccnntcncatctccccctcttcnntnnnnntcancncntcncnccattntncntccacntcaccnctccctatntnttctctnnactcanaccccnaccatccccnanntanctnccccctcantncaacncntctntcnnactccctctcnctcnactctnatctnctcnnntcantcactntctctnntctctccaccaccgtctactcatacttnntctcnccctctntcntcccncnctcncatnctcntctcatcntctcantccctctanntntncatttctnntccntcntttnctctacncnctcccgnacacactnctaccctcctcanttnctcccacnnctaccancnctntctcctcnntncnncntntactnctcgnnacnccnntntntntctctgntctcctnatcatcnnctcnnacncntnttcanatcnccanntatntctncanctaccc

SEQ ID NO: 203 - DNA, 1684 bp, Mus musculus
  misc_feature (4)..(1683): n = any nucleotide
tacncaccncttgtaccggctcggatcactagtaacggccgccagngtgctggaaagtctttctgtacttcctgcctggcacctgctttccctgcctctactngaccaagagttacacaagaangcgttctcccccgccagctcccacttggaagatttnacgaggngngnggctccnnggcgccatggccacacctacgctnnntgntgatnnnctancggaggccccacccccctnnctccttaccnctcntctnnnncccccccccccnntttcnccatnttcncnccccccctncntcntcttctcccctcnttctncntcncccattccnnctnacnnncntctncattccnncnnccctctctctcncncctctcccntctcctntattctttcttcaccctcncctccnanccntcctctctcntncctanttcnncccctntntccncatnctctaactcnncctcacactctcccctcnatccatcannnctcnctncctcctntctacaantcncncncctcnntntcncnctanctcnctncctctnnnacncncttcacccnntctntcnnnttccctcnactcccctctctctcctccnttnctctnntcnnttnctnccnatccnccnncncccctnantctccnnccnntaccttccncttcccatcctnctccctcnnntcccantctccnatnccnccttcnnctcntcccaacnnnctattncnnncncctccnntnncttcnccttcnccttnnatttttcnntntctttccctcctncacncnnctcntncnnttnntntcttnncncnatncctttncctcnnnntctcnctctccntctncntcctccnctctcntctcnnttcntcatccncncnncctncctncccncntntctctcnctcncnanccntctnnctcttatnccncacantanatcccacacctctnnnnttcccnccttcntcncncntctnccnncnnnccttnnctcttccncctccnanctnactacannctcnttctttccnnatcnaactnctcatctctnnccctccctcttttcntccaccttacctcncccttcnctttctccctnctaccacnncttnctcctnccntnctnccatncctntcctnnnannnntnnctcttctcctctctnctcatacntcnnttcaccatntcntctcttcncttactcactcactctnttacntnntncntactcctctctctctntactnccanctctccncncncttcctcatctccatcntccntcatcatntcnctnncnacacttctntctctntctcctctancctcnntctnctctcncatacttccnttctctnctcnctaatcccacnctntctcnnactctcnctctntccnnttctcctnttcacctctatccncnnttnnnccctctnactcnnctcnctncntcctcactnctnncntctctcncntctnnntntccctcnntncntcatctccntcatnaccnncttacnccatntaccctccntcctnctctctcantcttcccccctcncacanactccncttnntcctncnccccctcntctntntctcnntcancnatctctaaactaccnctnccnatctcacanacntcctctctntccntctcngnc

SEQ ID NO: 204 - DNA, 1455 bp, Mus musculus
  misc_feature (1)..(1454): n = any nucleotide
nnccgncccccttcnaccggctcggatccactagtaacggccgccagngtgctggaaagggcaagagcctccaccccttctacaaccagcacnatgcgcgagatcgngcacatccaggcgggccagngtggcaaccagatcggcgctaagttttgggaggngataagcgatgagcatggnatcgaccccactggcagttaccatggcgacagngactngcagctggagaggatcaatgtgtactacaatgaagctgctggnaacaaatatgtacctcgggccatcctagnggacctggagccgggcaccatggactcagngaggtcgggaccattcggccagatcttnaggccagacaactttgngttcgnccagagnggtgnaggaaataactgggcanaaggccactacacagagggngccnagctgcnggantctntcctagatgngnagagncaggancnttcccccnccccnnncctcccncncctncnncctnccctctccccccncncctctncttttcnnttctcctttttctnttccncnttnctnnnncttnctcncccncctttnnccnctntnttctcttnctcctccctttcctnttctcncntctnncncttctcnnncctntcncttcctntnncntnncnnnnntccnntcctcntnnctctnnnccnnnttncctnncnctttctcnccntcctcncctcnctcttcntnnttcnnncccnnncnctntcncntcnctctcnctttctcncntttanncnntcntctctcnttntnttctnctcncnccctcnntnctnctncnnctcctnctctncntcttcttctcncnnccntctnctncctctcntcnntctnctncnnctcntnnnnctctctcccctcctctctctncncctnnnntcncncnnntcncccctcctnnnnccnctccnctcnnctctctctcntnctcncnctnctctctctntctcctnntnctcntcncntnnctctcnnccctctctntnnttnccnncntcncnnttctctntcctcnnnctcnnctcntctcnccntcnnctcncntncntctccnnnntnctcttcncntcnncccnncttnncncctncntcntctncnnttccnnttctcnttcntctcntnnccctccncnnnntnnnnctcnnntcctctnnnnctncntctcctncnctctcntttcntccntctnnctccnnntnncctcncnncncntncccctccttnnnntcntcncntctnctnttntcnccnctccnnnctccntcnctnctnctcncntctntcnttccncncttcntctctttctntcnccnccnnctcctctctcnncttcctntcctcnannctnctntttntcnctcntcnnttctctntacnnctntctctcccnctcctncaccncnntctntcntnc

SEQ ID NO: 205 - DNA, 1735 bp, Mus musculus
  misc_feature (5)..(1734): n = any nucleotide
accgngncagcccnctnggnangcntggaacactagcaacggacgcnagacgacgcnggaaagnanggacaagnnaagaagcanccnnnccccctatcccccnccagnannaatngnaggannggggncaggagnagnaaaggaanagaagngcnaggtncagnnnaagccagangncngaagnnacagagagaacaaancagagcnatggaacaaacngngnnccaagtgaagacnagnagcagnacannngngagaagaggatcnaaagaanacngaagaaagaaggcccagacnnntaaantaagaagatnacaggacagntgnaganaanagaagggngccnnagnagncaatcgcacaaatcnccactaaagcacggaaatatgncangaangcangaaaacaagtngaacaannanctnggtannanantncnncanaanctannncnnatanccnaaaccnntnncnnncnancntnnntacnaacccnnncnaannaanacntnnganacnannttcaaccaaccatnnncatcnnanncacntcctaaccnaancncatngcnatcgntcntacatnntctctacaacngacctcnacaccaatcatcncnngatacatnatcancncntcnatnancacactagtacnntatatncatatctngntcaccanntcaaanatncgcacaccnnaacntnnntatactaccactcantctnangcacnntaccacatnacaaaccnataacacncctnncacacnccataacattnncancnctacacnntnnanagatatnacntnnnannntcnantcngccannaatncnctatnncaccntnatccatccaanatnnnnctctnnncgtacanaccccnnacatcctnntnacanntacncaataacangcnntanctcacannancnacacatnnacntcnnactntngngtaacnnatacngcnntcanccncaagncacccatcnannntanctatcacgccacananatctgnccnntatacccacntatannctatcacatannacntntaacannnatatnanaccacacnccaacncncnnatntatnncaacatacannggnannacntatntntcatatattnnannantnanncannnacnntnacacnncacncantactncnnacnnnntnanatcgtanatancctcgacgncantcactntngntcgactncacacntatctncntataatcancactataccatncacacgnctatntctntnnatcnantacacactncacnctacncgcataactctcccacatccnagacntacatntctcncaanacaacctctananaatnntacactcacacntncntantcnagctncantactannncntntcacctacncnnnnntgtacnctngangtatgnacnacannganncanatactataatacncnncnnatcgnacaaatanncacaaatcaanatntanacgncacnccaaatncanacnannnnnnnaatggacncaantcncanacactctacnaanaannatanacgnactcncacganacacgnnntnagacatntancacnaccnacnncnantncatcnnctaagncatnnntacaacccnncacannncnntctcagannncgncatacantnnatnacncnnaagnccattancnncannacgacanngnna

SEQ ID NO: 206 - DNA, 731 bp, Mus musculus
  misc_feature (10)..(731): n = any nucleotide
tttccccccntcnccggctnggatccctngtaacggccgccagtgtgctggaaagccgnaagcgcagagcagagaggagcagcgggcttgtcgnnggnggcggnggcggaggccgccatggatcgcgatgaggaacctctgtccgcgaggccggcgctggagaccgagagcctgcgattcctgcacgtgacagngggctccctgctggccagctatggctggtacatcctcttcagctgcatcctactctacattgtcatccagaggctctcccttcgactgagggctntgaggcagagacagctngaccaagccgagactgntctggaacctgatgttgnngttaagcggcaagaggctttagcagctgctcgtttgagaatgcangaagatctaaatgcccaagtngaaaaacataaggaaaaactaagacagcttnaagaagagaaaagaagacagaagattgaaatgtgggacagcatgcaagaaggcagaagttacaaaagaaattcaggaaggcctcaggaagaagatggtcctggaccttctacttcatctgtcatccccaaaggaaaatctgacaaaaagcctttgcnaggaggnggntatnaccctctgacggntnaagggnnntnnaccctnctnctgnngacctnnacncanggncccatcatntgncnncttnnaacttnnnactctttgnnngnngnncnctcttnncnttanccnnn

SEQ ID NO: 207 - DNA, 1213 bp, Mus musculus
  misc_feature (30)..(1209): n = any nucleotide
cgccccccttggtaccggcttggatccctngtaacggccgccagtgtgctggaaaggcatgaccaagaagatgatggacaaggnggagtttgtccatatgcttaatgctacaatgtgtgctaccacccggaccatctgcgccatcctggagaactaccaggcagagaagggcatcgctgngccagagaagttgagggagttcatgccgccagggctccaagagctgatcccgnttgngaagcctgnacccattgaccaggagccatctaagaagcagnagaagcancatgaaggcagnaaaaagaaagcgaaagaggtccccctggagaaccagctgcagagcnngnnggtnactgaggcctgagcactnccagacttacttactaagnctgctgngntcccaggccttgccctgncacagngaggccgcgnannactcctcnncctgccncctctncntnccagctcctgccgannccnnctgcgccgacngngtacnnnccccttctcctncnacncnncncctncnctnctttcccntctccncccncncccncnnccccccttcccttccncnccctttcccnnncntnccttcntcccncncncnccnctcctctccctttnntnnntcctncnctcnctccntnnctcnctccnncnnncccctcctcnncctctcncttcctntncntcctnnnnnnctncnnctctcntnccnncnnccnctnctctccttcctcntctcctctccntcncnnnntctttcttcnnctctnctnctnnnncnnncnncnncntccncncnctncncnctccnnncnnncttncntctncnccnnncnttctcctcctcctcctccnncnccncntntccnccncccnnnnccnctcctcnnncccnctnnnnnntnttntctctcncctcctctcnnctntccccccnnccttncccctccntcctnntnctccccntcctncctctcnnntncttcnnctcctnttctcacntttnctcnncnnncncttcctccnccctttcccccttcnctccccnccnntttctcctctctncctcactcctcttctcttnctcctncttctccnccctctcctccctncctcccnnnttnctttccnccctctcntctctccctccctcccncnttctcnntnctnntctttcnctctcttcttcntcccctccnntccc

SEQ ID NO: 208 - DNA, 1456 bp, Mus musculus
  misc_feature (3)..(1456): n = any nucleotide
ccnnactncacaacctctttcnaccgcgcctatncncanccacctannntanactgggacacgnacagatgncgcatgngaaaagcaactgacatacnnccgannnactaanaccttctagtncanccaatttttntncctnntnaacaanaacactcactntttnccnantnctactnnntgtantcnanatntnnnancttctnngntctantancttttnnctccanncctnctcctatnnancatatcccnatctnctccactcnntntanntacccactcctcctcncccancnctttcccnctatccanactcnntctactcntcnctcccttcnncncanttcnccatnttntctcctnatcnntttnnntccctcntnccnnntnnacncanccttctccccttnttnncnnncttncntcttnncnccatcccctntcccnctcncttcantntntatccccctntnncccncccntannattcntcnacctaactactcntctcttntttcntccannnncnccnccccctnctctnactcntccntcnccttatnatntctccatncactnnannntcccactnnacnctcattnntatatccntcnnctcttccaccncnctnctnctacannnnctcncctactctcncctttacnatnctacctnnntanactnncntcnnanntntntattctncaanactntctaaccccctantctctcncnttnncncctnctnnnnntantctcntnatntttctannccttcctntcnatnanttctcatctacntcnnnntncntcnttttnntcattcctcntnaatctcctnatnntaanttatcntcccaancnnntnatcaatntactnntctnancactctctncctcctntccnccacncnantctactnaacnntncctcncactntnntnttcanttncananatnacntnntnccntatcttcantctccnactctctacncactcttntcanatccacttcatcctcnncntcttnatctcncnnnttattanccntacctctntcntcntatnntnccttcanttccctacccttcactcttctacnnctccncntctcctcncancctcacctctctcctctcncactnnnnctctnnatacnccntantcatcnctccacntctacntantntntntatacactnnacnncttcantcactnnttacatcattannntctctccnctatncccacttantccctcactatactatctatctctcancctnatttnctcctccnnctatctncntannctctcacttttctcatnatctccancntntctnnacncanatctcntcnatctnatnactctctcctntccatcnctcntctntctncatctancntattcacnccgactnctncctcantncacanattannnn

SEQ ID NO: 209 - DNA, 427 bp, Mus musculus
aggcaatggcggacgtgtctgagaggacgctgcaggtgtccgtgctagtggctttcgcctctggagtggtcctgggctggcaagcgaatcggctgcggaggcgttacctagactggaggaagcggaggctgcaggacaagctggcaacgactcagaaaaagctggacctggcctgagcacgcgctgcagcccgagtccgccgggttctcactccctaagcccaacgcagcccggatcgtgggagccgcgcgacccaggagtcgtccttgcacggcttgcaagaacatggcttgcttcagaaagaaaatagttttgtcttctctaacaacttactttcagcttgtcgaagatgaaaataaaaagcactggagagaaataatttcttgcactttatgaatctatttttaaaataaaaaaattaaacatc

SEQ ID NO: 210 - DNA, 558 bp, Mus musculus
tagtaacggccgccagtgtgctggaaagggactttgttcagatctttttctgatcgtgcatcagctgctggttcattatagccttctcttaagcactgaataagacaaaaaatgaatcttggaagaacaaattcagacatcatcagtaagtctttggggacacagggaatatttgaacttgatttaatctgatgtcttctacaaaacccgctctcccgcattacgttgctgtccccgcagtcgcaggcgcccccggcctggctgcggaacatgttgaagtcgtgtccagtgtggtcgccctggtggaagcactccgcgcacagcgacatgcagggggagatgccgcacgtccggcagcggtaggccacgaagttggccgtccagaccaggccgcagagcgccgcggggtcgtaggcccgcacggccgcgcagaactcctcgtagccgccgccgccggccagaaggcacttacaccactccagggcatcctcctccgcctcgcccgggccgccgccgccgccgcccgccgcctcctcgccgcccgcagccccggccagc

SEQ ID NO: 211 - DNA, 807 bp, Mus musculus
  misc_feature (60)..(806): n = any nucleotide
aggcgacgcgcgcatggaggccggctgaggagcgccgccgcctctctcggtaaggactgngtctgtgtccccaggcatcctacatcaatcaggaagctgctgtccagccatggagggagaggagaagccagctcaagaggctgacgtggaacctgtggtaacagcaggcacctcagaagcagtgccaagggtgcttgctggagaccctcagaacatctctgatgtggatgccttcaacttgctcctggagatgaaactgaaacgacggcgtgaacgccccaaccttccacgtacagngacccagctagnggccgaggatgggagcagggtgtatgtggngggcactgctcacttcagtgatgatagcaagagagatgtagtaaagactatccgggaggtgcaaccggatgtggtcgtggnggagctctgtcagtaccggggtgtccatgctcaagatggacgagaggacgctgctgcgagaggccatggaggtcagcctggagaagctgcagcaggctgtcaggcagaatggnttatgtctggactcatgagatgttgctgcntgnaggtgtctgctcacatcactgagcagctgggcatggcccctggtgncgagttcagggaggccttcaaagaggccagcaaggtaccattctgcaaattccacctgggngaccgaccaatcccagncncctttaagaggnccattnctgcactctccttctggcanaaagtcaagcttgnnctggnncctgngcttncttgncngacccaatcannnnnngncnacnnnnnnnnncttnaant

SEQ ID NO: 212 - DNA, 672 bp, Mus musculus
cttgtaacggccgccagtgtgctggaaagtgaccggctaccagtgcagagcatcccaggccccaacctttcctggacagttggacctcttcctgagagaccccggaacgttttctaccttacccttgggagaagagcctctggctgaatggcagctgtggatgacttacagtttgaagaatttggcgatggagctacactgttagcagcaaatccagatgctaccacgataaacattgaggaccccagtgtatcttttggccaccaacccagaccccccgggagcgtggggcgagaagaggatgaggagctgctggggaacaacgactctgacgagaccgagttacttgccggtcagaagcgaagctctcccttctggacattcgaatactatcagacattctttgatgtggacacttaccaggtctttgacagaataaaagggtccctgctgccggttcctggaaagaactttgtgaggctgtacatccgcagcaatccagatttctatggtcccttttggatatgtgccacgttggtctttgccatagcaattagtgggaacctttctaacttcctaatccatctgggagagaagacataccattatgtgcccgaattccagaaagtgtctatcgcagcgactgtcatctatgcctatgcctggctggtc

SEQ ID NO: 213 - DNA, 876 bp, Mus musculus
  misc_feature (5)..(870): n = any nucleotide
cccanacctctntcgncacgagctcggatccctagtaacggccgccagtgtgctggaaagcccgtccnnagcgctccggcgagctggccttngctgcagtngagccgggctngnactatgnagngcctggccatggacctgcgggtgctgncccgggagctggcgctctacctggagcaccaggttcgcgtcggcttcttcggctcgggcgngggcttgtcactgatcttgggattcagcgttgcctatgcttgctactacctgagtagcattgccaagaaaccccagttagngatnggaggggagagtttcagcncgcttcctacaggaccactgtcccgnggtgacagaaacctattacccgacggnctggngctgggagagccgaggacagacattgctgagacccttnatnacttncaaacccccggtgcaatacaggaacgaactcattaaaactgcagacggaggacagatctcactggactggtttgataacaataacagtgcgtactatgnggatgccagcaccagaccntactatcttgctcttgcctggcctnactgnaacaagcaaggaatcctacatccttcatatgatccatctcagtgaagaattnggatacaggtgngnggnttttaataacanaggagtagcangagaaagtctcttgacaccaccggacttactgctgngcgaacactgaagaacttggaggccgnngtccaccacgtgcacagcctgtaccctggggcntcctttnctggcancnggcgtatcaatggggagggaaatgctgcttttngnanttacttngggttaaaaattgggtccaaaaanccccntcttganggcntncctgcnaccnntttccc

SEQ ID NO: 214 - DNA, 858 bp, Mus musculus
  misc_feature (583)..(858): n = any nucleotide
ttcgacccccttggtaccgagctcggatccctagtaacggccgccagtgtgctggaaagggcgcagccggcctagcgaggtcaacatgccggtcgccagaagctgggtttgtcgcaaaacctatgtgaccccacggagacccttcgagaagtcgcgtctcgaccaggagctaaagttgattggagagtatggactccggaacaaacgtgaggtttggagggtcaagtttaccctggccaagatccgtaaggcggcccgggagctgttgacgctggacgagaaggatccccggcgtctgtttgaaggcaatgctctcctgcggcggcttgttcgcattggggtgctggacgagggcaagatgaagctggattacatcctgggactgaagattgaggatttcttggagaggcggctgcagacccaggtctttaagctgggcctggccaaatctattcaccatgcccgtgtgctcatccgccaacgtcacattagggtccgcaagcaggtggtgaacatcccatcctttattgttcgcctggactcgcagaagcacatcgacttctccctccgttctccttatggnggcggccgtccaggccgagtgaagaggaagaatgccaagaaaggccagggcggggctggagctggtgatgatgaggaagaggattaattcttggctgaactggaggattgtctagttttccagctgaaaaataaaaaagaattgatacttggaaaaaaaaaaaaaaaaaaaagggcngccngctcgagcatgcatctagagggccctattctataggggcacctaaatgctagagcttgctgacaagccttgactgngncttctagtgncngncn

SEQ ID NO: 215 - DNA, 1239 bp, Mus musculus
  misc_feature (2)..(1238): n = any nucleotide
tnngncccccttntaccggctcggatccactagtaacggccgccagtgtgctggaaagcggcggctctgagcgccggagccgccccgggacatcatggcggaggtctgcgagcccacccgcccctcggaggacgaggacgaggagcgggagccgctgctgcctcgcgttgcctgggcccagccgcggagggtcgcgcccgggagcgccgngagaatgcaggcggacgagggcgcggatgtcctccgcgagcccgctaccgacgagccgccggcggtgtccggngaagggtcganctnngcgagcttgtccnnngagctggatntngacccncnctacnctctcantttacnntnatattntttnncgattcacnattnttctccttcntgnctntncnnccnntcnccnccntncnctctntctgncntcctnnntccctntnntcctccctctnttntntnccctcancccccnnnnnccncncttcccncncntctcccttctctcncctccnccccncntnntnnctcctcnncnnccnntcncnnnntcnntnctnctnccctnctcccnnnctcnnnnnncccnccctcttcntntcnctccnnnccncntctcnncctncccccctnccntctcncnnntnnnccncnntcnccctctctcccncctttctntcttctcncnntcctnctnccnantntctncccctnacctcnnccntctcnntnncnncttttcnncctctcccnnnnntcctttctcancccttctntcctnnncctctnnncnctnnntctccnctccctcccnnccnctcttcncctnncctctctcctntctnttntctccntntcntcctcnncnccctnttcccncatctcttccctttctntcctctccnttncctncccacgtcttccntnnanccctnnnannnnntccttctcttncntcntcccccctcncnnncncttctnntnctcnnccnnnntacntntctccccncnntccctttcttctncnnctnnnnctcnntttcncctcntcnnnctcctttcatnctctcctccnncatntcctcnnctnctctctctctatcncctcnctntccatcncnctctntcttcncntnctncctcctccncncctcnnctctcnnnnnccctctntnnnnctcntccctcatctnctctctctccttctnnncncntcgtcnctntccnttcnctnnntnc

SEQ ID NO: 216 - DNA, 1261 bp, Mus musculus
  misc_feature (716)..(1261): n = any nucleotide
cccccttcttggtaccgagctcggatccctagtaacggccgccagtgtgctggaaagggagcagcaaacggccggcggcaggcgccgcgcggggggcgggcggcgcggaggcggcggtggccatggccgaggcgtcgccgcagcccggacggtacttctgccactgctgctcggtagagatcgtgccgcgcctgccggattacatctgcccaaggtgcgagtctggcttcattgaggagcttccagaagagaccaggaacacagagaatggctcagccccctccacagcccccaccgaccagaaccggcagccatttgagaatgtggaccagcacctgttcacgctgccacagggatacagccagtttgcctttggcatcttcgacgatagctttgagattcccacgttccctcctggagcccaggccgatgatggcagggaccctgagagccgacgggagagagagcaccagtctcggcatcggtacggggcccggcagccccgtgcccgcctcactgcccgccgggccactggccggcatgaaggtgtccctacgctggaagggatcatccagcagctcgtgaatggcatcatctctccggctgctgtgcccagcctgggccttggtccctggggcgtcctgcactcgaacccaatggactacgcctggggggccaacggcctggacaccatcatcacgccagctccttaatcngtttgagaacacccggnccccccacttgcagacaangagaagaattcaggctnttccccncggtcccagtcacagangaacacngtgggcttcanggcnttgagtgcccaatggtgtnaagnaagactattccnctgggtnanaaatntgccggcaaantgcccttgnaaccncttgttcccnganaagctgcatcgnncccntgcttngancnancatnacagcttgnccgggctgncntaaganccctcnntggacagaaacncagcccccaatcccccagctttgaccggggaaggttnttcctcgccnngncttaanccttctccanttcnccacnattggaaccccccaagnaaaatntggngccnncttgcnaaccccccggggntgngantccccnccccggggcccaaaacgccnctttccccccccngcttgggnaaaaaggtttccctggggccgaaacntgggggcccantttgnnnnntttnattcanggccncttttggggntccnggncnntccnnggnngnttttttttn

SEQ ID NO: 217 - DNA, 804 bp, Mus musculus
  misc_feature (125)..(723): n = any nucleotide
atggcggctctgcccttcctcatcatcgagacaagcaccattaagccttaccgtcgagggttttactgcaatgacgagagcatcaagtatcccctgaaagtcagtgagactataaacgatgctgngctctgngcggnggggatcgtcatcgccatcctggcgatcattacaggggaattctaccggatctattacctcaaggagaagtcccgctccaccactcagaacccgtatgtggcagcncncnataagcaagtggnatgcntcctttnngntgtgccaattagcaagtccttcanngncatcgccaaagtgtccatcgggcgcctaaggcctcacttcctnagcgtctgtgaccctgatttcagtcagatcaattgctccgagggctacattcaganctacaggtgcagaggagaagncagcanagtacaggaggccaggaagtccttcttctcgggccacgcctccttctccatgttcactatgctgtatctggtgctctaccttcaggcccgcttcacctggcgcggggcccgactgctccgccccctcctgcagttcactttgctcatgatggccttctacacgggattgtcacgggtatctgactacaagcatcatcctagcgatgtcctggcaggatttgcccaaggagctctggtggcctgctgcatagtgttcttcgtgtccgacctcttcaagactaagacgagcctctcnctgcccgcccctgcgatcaggagggagatcctgtctcccgtggacatcatcgacaggaacaatcaccataacatggtgtag

SEQ ID NO: 218 - DNA, 541 bp, Mus musculus
  misc_feature (231)..(231): n = any nucleotide
gggaagctgaagcagttcgatgcctaccctaagactctggaggacttccgggtcaagacctgcgggggtgccacggtgaccatcgtcagtggccttctcatgctcctgcttttcctatcggagttgcagtattatctcactacggaggtgcatcctgagctctacgtggacaagtctcggggggataaactgaagatcaacatcgatgttcttttcccgcacatgccttgngcctacttgagcatcgatgccatggacgtggccggggagcagcagctggatgtggaacacaacctgttcaagaaacgactagacaaggatggcgtccccgtgagctcagaggctgaacggcacgagcttgggaaagtcgaggtgacagtgtttgaccccaactccttggaccccaatcgctgtgagagctgctacggcgctgagtcagaagacatcaagtgctgtaacagttgtgaagatgtgcgggaggcctatcgccgtcgaggctgggccttcaagaacccggacaccattgagcagtgtcggcgag

SEQ ID NO: 219 - DNA, 742 bp, Mus musculus
  misc_feature (208)..(742): n = any nucleotide
cctatcacaattcatatttattgccctgggctgggctggctgaggagaggatatgggtagttgacaggctggagggtaaacccacaggagaagagagggcaggacaagctgtggggaagggagagagctacgttgtcttccctaggtcaattttcttctggatggcccgagcggagtggtagatgagcgagtcaatgaggccggccantgtaaacatgcccccaatgatggcgcacacacctgtcaggaagtgggtgaaagacctgtgtttctccgtcagcttcaccatcattggtgagagctcatacagcacaaagacccccggnaggccctggtcacccaggagcccattggcaaccttctcgngcctggtcacgganaactggttagtcctcagcacctccccgtccaccttcatgtacactgngggcaccacnttcacaaagtactggaacatcatggaggcttggggtgcagtcacgttggtgtggnctagggggttcacgattcctggatagtcctccccgaatgacaggtgcttgatgtagtgtgncatgttgatattgtcaaggccaaagctntgcaagtcatgaacatgcacatgagactgttggaagctcttnccaggggcaaagtggaagtttccagccaccttgttgacctccaagaagccnnacacctggcagccttcattcttctgctcctgcatcttctggctgaagccctttcgccgacactgttn

SEQ ID NO: 220 - DNA, 777 bp, Mus musculus
ctggaaaggggcgttgagcagctgggaccggagttgtgctcaccggggtcgggccaggtcgctgctgctctggccatggccgaggcacgcgcatctcgctggtactttggggggctggcttcctgcggagccgcttgctgcacgcacccgctggacctgctcaaggtgcatctacagacccaacaggaggtgaagctgcgcatgactggaatggcactgcaggtggtgcgaactgatggcttcctggcgctctacaacggcctgagtgcctcgctgtgcaggcagatgacctactctctgactcggttcgcaatctacgagaccatgcgggactacatgaccaaggactcccaggggcctctccccttctacaacaaggtgttgctgggcggcatcagtggtttaactggaggcttcgtggggaccccagcagatttggtcaatgtcaggatgcagaacgacatgaagctgcccccgagccaacgacgcaactactctcatgccctggatggtctgtaccgtgtagcccgtgaagaaggcctgaggaagctcttctctggagcaactatggcgtccagccgtggggccctcgtcactgtgggccagctgtcctgctatgaccaggccaagcaactggtcctcagcactgggtacctgagtgacaacatattcacccactttgtctccagtttcattgccggcggatgtgccacatttctgtgccagcccctcgatgtgctgaagactcgcctgatgaactccaagggcgagtaccac

SEQ ID NO: 221 - DNA, 777 bp, Mus musculus
  misc_feature (634)..(775): n = any nucleotide
tgcagggaagacagaaaggcctcccaggccacttggtttattagatcctgaagagaggtgtaggcagtgcccctgggccgctgccacctcctgggggaggacctgtgggaggcacagggccgaacctcgttttgatacacacaaccccatttgagggaaaacaggctgcttcgaagcctgagggatggtgaggggtgatgcctgccatacaggaagccaggtcctgggagggcacaaacgatgaatccatcactgccccagctctgccagcatgcccacctggccctggggaagccaggcaagggagggcacaggcgtgtgagggacacagacagttcctggtgacggcagtagctgctgagcaggagggttcagcaaaactgaccattagagcagccaaggctgcatataggaggtgcgctcgggaaccccaggcactttctctggactccacggtcatggcttctgctggtgatctgcactgcctgcctgtcccctctcctgaaggcactaccttccagaacacagcacggtggtccctcttgtgacaaagtgcttgtgtgtgtgctctagtcatttggtaagcagaagctgccacgggccataccctgccacactacccaagttctgggcnggaaactgctccctgcacagagggccagcgggagcaggaaacgaactcaacttcgctgggcttgccaggcanggcacggtagcagccagggaggttgggacagtgacancagncaggcagacttttttgnggaataatngnac

SEQ ID NO: 222 - DNA, 274 bp, Mus musculus
atgcaccaccagtctgttctgcacagcggctactttcacccactgcttcggagctggcagactgctgcctccaccgtcagtgcctccaacctcatctatcccatctttgtcacggatgttcctgatgatgtccagcctatcgccagcctcccaggagtggccaggtatggcgtaaaccagctagaagagatgctgagacctctggtggaagctggcctgcgctgtgtcctgatctttggcgtccccagcagagttcccaaggatgaacagggct

SEQ ID NO: 223 - DNA, 467 bp, Mus musculus
  misc_feature (187)..(467): n = any nucleotide
ttcgcccacgccttggtaccggctcggatcccttgtaacggccgccagtgtgctggaaagcaaccgcaacctgaccttgaccaagaaggaacctgttggggtctgtggtattgtcatcccctggaactatcccttaatgatgctgtcctggaagacagcagcctgcctggctgccgggaacaccgtnttgatcaagcctgcccaggtgaccccactcacagccttgaagtttgcagagctgacactgaaggctggcattcccaagggtgtggtcaacatcctcccaggatcnggctcgctggttggccagagactctcagaccaccctgatgtgaggaaaatagggttnacaggctccacggaggtggnaaaacacatcatgaanagctgtgccctgngtaatgtnaagaaggnctncctgcagctgnnnntaaagncacccttntcatcnntcctgctgnnncctn

SEQ ID NO: 224 - DNA, 894 bp, Mus musculus
  misc_feature (10)..(893): n = any nucleotide
gggggtgcgnggnggnggcccgtccccngcctnccctncgngcgccgggtttcgcccccgcggngtcnnnccccnnngantntacnncncgacgagtntgagggccgctgcngtgagccttgaagcctatggcgcgggcccngntggagccgccgnaggtgcatatcttgntggtantagcaaatattcanacnataactttgaatgccnaagtgganaanggttccatgtgaanagcagttgatcatgggtnanntnggtcctganagatgggcgagtgccgttccgaanggacagtcgatggcctccgatgacctcggncgatcanaaagggagtcnggtttanatccccgaatccngagttggcnganatgggcgccntgaggcgtacaaatgcggtaacnngaccgattcctgagaanccnncnngagnnncgganagagntctnttttntttgcgaagggcattngcgccctgnaatggnttcagnncctanagagggntccntgccttgtaaagcgtcgcngttccngangngtttctngtgagctctnctnngcccttgnaaatcccgtgnnanagggtgtaaattctntgtcncgggcncnaacnnatattncttatgcatgatcntcnaanntttaaccancctnatggccatgttngcnanaaattgnacctctaangttaaagttccnncnattncctnnannccttntaanctttnnnngnaataaatngatttggncctttttaaatgggccttngggtccngnnnccggnanntttgngntgcnntccnaaaaacccncatggnnttngnaacnttccncnnnncccnntnntnttttntaacntaantggtnctcctnctcncncttctttctttannc

SEQ ID NO: 225 - DNA, 506 bp, Mus musculus
  misc_feature (21)..(503): n = any nucleotide
tgcagggaagacagaaaggcntcccaggccacttggtttattagatcctgaagagaggtgtaggcagtgcccctgggncgctgccacctcctgggggaggacctgtgggaggcacagggccgaacctcgttttgatacacacaaccccatttgagggaaaacaggctgcttcgaagcctgagggatggngaggggtgatgcctgccatacaggaagccaggtcctgggagggcacaaacgatgaatccatcactgccccagctctgccagcatgcccacctggccctggggaagccaggcaagggagggcacaggcgtntgagggacacagacagntnctggngacggnagnatctgntgagcaggaggtttcagcananctgancattanagcanncaaggctnnatatagnangtgcnctngnnaacctnanncnntttntctgnactnnangnnctgncttctgnngnngatcngnnctnnntnctntnccntntnnntnagg

SEQ ID NO: 226 - DNA, 1041 bp, Mus musculus
  misc_feature (238)..(1037): n = any nucleotide
agctcatgttggacagtcgtgtgaggagctatggagcacagcagtaatcgcccagaggacttcccgcttaacgtgttctctgtcactccgtacacacccagtaccgccgacatccaggtgtccgacgacgacaaggcaggggccactttgcttttctcaggcatctttctaggactggtggggatcactttcactgtcatgggctggatcaaataccaaggtgtctcccactttgtanatggacccagctcctcggacccatccttctgtcggtcggagtgacattcatcctgatcgctgtgtgcaaattcaaaaygctatcctgccagttgtgctcagataacgaggagagggtcccgggactccggaccaggacttccsgaggacagtcgttcgttttcactggcatcaatcagcccatcaccttccacggggccaccgtggtgcagtatatccctcctccttacggttctcaggagcccctgggaatgaacgccacctacctgcaacccatgatgaatccttgcggtctcatacctcctagtggagcagcggctgccgcaccaagtccccctcagtactacaccatctaccctcaagacaatgctgcgttcgtngagagntgagggcttctctcctttcgtgggcactggatatgacaggcccgactctgatgctgaccagctagaagggacggagttggaagaggaggactgcgtatgtttctctyctccaccgtatgaggagatatacgctctacctcgctagagactgcaatgctaaggggacggacatttaagcccctgngatgtgatacttggagagtttatcgctgagttcttcagaagttaggtgtcaaagcagctnaggagatcttacagatgtcattnaaggngggaaagaagtgcccngagactgctaaattaagctgccctggttaaattcccctctgctctggttttgaattctctcagctaagaaaccctctgcagctggagagtcgctctgagatagagagatttnggagcccacgcagngcct

SEQ ID NO: 227 - DNA, 1049 bp, Mus musculus
  misc_feature (300)..(1024): n = any nucleotide
atagacatgtctagttttttattactagttatcatccaagtgaaatgtccctgaggcataatatgaatcacaataataaaccaagattgttttgctgatatccttgaaaaacctggactcttctgacagagagtaaaagcaatcccacatataagcacagtaccaaaaccttcaagacctgacaattcctgtatctctcttgggggcagccacctttaccttgagtggcctttgatctttgtaaaactgttttctttctaattctttggacacctcctaaagtctgcagctttgggctgnggttggtgacaccgttcagacttaggaagctgaaggctaccagcttttctttgttcaaatgaaggtgagagctacgcccgggggcggaggkagrgggagacgggatgactgatcatctccatgattcatgactacaaagcgacacggcagnatggctctgnggaacagatcagaggcatacctgtgttccataagcnactcagctgtacctgcgttctgtaagccactcggctgagctagttcgngcctgggaatcacagcctgggggngggcagagggagcaggcgctnaccnatgtacattacgccttgaacttcanccttgcaaaggaaaacnatcangagcaggggtcactntaactcggnggacagcacatggngacattcattatcctcctgntacctcgtccacagtaggatcggccacccaacactattctaacatggggcactgtttcatccnactctacttattcatttatttcttaatggattttatttctttaacaaactcttnaagatccaaaggcttccagngagaccanataanagtcnaagttgtctaanataagtnactctgctgcggaaggttcagngccgtgcagggaaatttctacctgagcctgctctcttccctgcttgcttgccagcctccctttnatcantctnagagctgccatggctgcccggatttaaaactaanacaatctttnaaaactaagatntcctttaaatggatattccgcatgg

SEQ ID NO: 228 - DNA, 1059 bp, Mus musculus
  misc_feature (799)..(799): n = any nucleotide
cgccgcctgagcgcccggcccgaccccgccatggggtgctgctatagcagcgaaaacgaggactcggaccaggatcgggaggagaggaagctgctgctggaccccagtagcacccctaccaaagccctcaatggagccgagcccaactaccatagcctaccttcagctcgcacagatgagcaggccctgctttcctccatccttgccaagacagctagcaacatcattgatgtgtctgccgcagactcccagggcatggaacagcatgagtacatggaccgggcaaggcagtacagtacccgcttggctgtgcttagcagcagtctgacccattggaagaagctgccaccgttgccatctctcaccagccagccccaccaagtgctggccagtgagcctatccccttctctgacttgcagcaggtctccaggatagctgcgtatgcctatagtgcactttctcagatccgcgtggatgcgaaagaagagctggttgtacagtttgggatcccatgaagagaggggccctaggacagctcttccctcgtcttcaccccgtctccaccccacctcttctggcccccagcctcactgtggctctctacagtacctaacctgctactaatcacggagaagaatgtggagggaaagaacaaggctggaggccggagcaagtgaggactaagcaagggaagggaggaccgattgccatcggccttcatgctctggttagggtgaggttggggccaagaggacagggcctggcagatcttcagtcattgggaagatggagataccnctgtaggggtgacaccgggagacctaggagatcccttcctgccctctttctcttggcctccgattcactcctgtccccttccctgacttggtgctcacaggcacctcactggggattatgaccagggtctagacgagcttgagtctgaattgagtttgtatttctagcaccctgggtttttacatgtttgctctttttgtttttgtttgtcacccctcgataaagaaagtatattcatttgaaaaaaaaaaaaaaaaaaa

SEQ ID NO: 229 - DNA, 681 bp, Mus musculus
  misc_feature (564)..(564): n = any nucleotide
aggttcctgtcgtttatgctgcgcgccgcgagcatcttcgtgaaggacgctgtgctctastctggcttcacgctggatgaggccgagcgcctcacggaggaagagttgcatatcatcgcgcaggtgccgcctccacccgccgctgccgccgttggctacgctggctgccgtgtggctgtgaccttcttcctctacttcctggctaccaactactactggattctggtggagggactgtacttacacagcctcatcttcatggcctttttctcagagaagaagtatctgtggggcttcaccatctttggctggggtctgccggctgtcttcgtggctgtgtgggtcggtgtcagagcaaccttggccaacactgggtgctgggacctgagctctgggcacaagaagtggatcatccaggtgcccatcctggcatctgttgtgctcaacttcatcctctttatcaacatcatccgggtgcttgccactaagcttcgggagacmaatgcgggccggtgtgacaccaggcagyagtaccggaagctgctcacgtccacgttggtgcyngcgccactctttkgtgtccactacacccgtcttcatggccttgccgtacacccgaggtcttagggacactggggcagatccagatgcactatgagatgctctttaactcctttcagg

SEQ ID NO: 230 - DNA, 860 bp, Mus musculus
aggcgctgcctaccagagcgcagcatgacggccatcggcgcgcaggcccacaagctgttgggccttaagaggccccaccggtctttctttgagtccttcatccggacactcatcatcgtgtgcactgccctggctgtggtcctttcttcagtctccatctgcgatggccactggctcctagtggaggatcatctctttgggctgtggtacttctgcaccatcggcaaccacagtgaaccacactgtctgagagacctgagccaggcccatatgcccgggctggctgtaggcatgggcctagcacgcagtgtggccgccatggcagtggtggctgccatcttcggcttggagatgctcattgtgtcccaggtgtgtgaagatgtccgctcacggcgcaagtgggccatcggttcctacctcctcctggttgcctttatcctctcctctgggggcctcctcaccttcatcatcctgctcaagaatcagatcaacctcctgggcttcaccctgatgttctggtgtgaattcactgcctccttcctcttcttcctcaatgccgccagcggccttcacatcaacagcctcactcagccctgggaccctccagcagggaccctggcttacagaaagcgaggttatgatgggacttctctgatataggacctctggctgagattgaataggacaaccaatgaccttgacactgcctcttaggcacttagctctagcaatgccctggaagtctcttcagctgagctccagggcaaaggcagaagggtgcctctgtacgacggcacagtgagctggataggttagtcatgctattaaaatctcaatttgaaagtaaaaaaaaaaaaaaa

SEQ ID NO: 231 - DNA, 555 bp, Mus musculus
  misc_feature (457)..(457): n = any nucleotide
agcagactcaggaagaaaccatggtgctctctggggaagacaaaagcaacatcaaggctgcctgggggaagattggtggccatggtgctgaatatggagctgaagccctggaaaggatgtttgctagcttccccaccaccaagacctacttccctcactttgatgtaagccacggctctgcccaggtcaagggtcacggcaagaaggtcgccgatgctctggccaatgctgcaggccacctcgatgacctgcccggtgccctgtctgctctgagcgacctgcatgcccacaagctgcgtgtggatcccgtcaacttcaagctcctgagccactgcctgctggtgaccttggctagccaccaccctgccgatttcacccccgcggtgcatgcctctctggacaaattccttgcctctgtgagcaccgtgctgacctccaagtaccgtaagctgccttntgcggggcttgccttctggccatgcccttcttctctccttgcacctgtacctctttggttttgaataaagcctgagtaggaagaaaaaaaaaaaaaaa

SEQ ID NO: 232 - DNA, 1013 bp, Mus musculus
aggggcttcggacccggaagtggcgccttgggctcccggcggcgccgcggggatggcgggagccggagctggtgcaggagctcggggcggcgcgccggccggagtcgaggcccgcgctcgggacccgccacccgcgcaccgcgcgcaccctcgccatcctcggcccgcggctcagccgtcggcgcgcaggatggacggcggcccgggcgccccgggctccggggacaacgccccgaccaccgaggcgctgttcgtggcgctgggcgcgggcgtgacggctctcagtcacccgctgctctacgtgaagctgctgatccaggtgggtcatgagccgatgccccccacccttgggaccaatgtgctggggaggaaggtcctctacctgccgagcttctwcaacctatgccaagtacattgtgcaggtggatgggaagatagggctcttccggggcctgagcccccgccttatgtccaacgccttgtccactgtgacccgcggcagcatgaagaaggttttccctccagatgagatggagcaggtttccaaacaaggacgacatgaagacctcactcaagaaagttgtgaaggagacatcgtatgagatgatgatgcagtgtgtatcgcgaatgctggcccatcccttacacgtgatctcgatgcgatgcatggtgcagtttgtgggacgggaggccaagtacagtggtgtgctgagttctattgggaagatcttcaaggaagaggggctgctgggattcttcgttggcttaatccctcacctcctgggcgatgtggttttcttgtggggctgtaacctgctggcccacttcatcaatgcctacttggtggacgacagctttagccaggccctggccatccggagctacaccaagtttgtgatggggattgcagtgagcatgctgacctaccccttcctgctcgttggagatctcatggcagtgaacaactgtgggctgcgggctggactccctccgaattcccctgagttcaagtc

SEQ ID NO: 233 - DNA, 472 bp, Mus musculus
gagcagtgttggggtggcatcccccttcctgcctagaggtactggagtccatcttgtactcaggcagaggcaggctgcagaggcaaacgtcactcagtggcaaggcttccctgcacctctagcccagctcatcctgccagtcagccagaagcacccccgccccccacttcctgctttgtaaattgggcgccatcacacctgggccatgggaggctggcgctatgttcccaacactaattttcttatacaagggtggtgccttctcctgaataggaaatcatgttctcctcagaccatcccctcatctgcttgtctgtgctggtgacgccaggtgtgagggttcagtcactgtgctgggtgcgaatacgcacaggttacataggccgacatctagtcctcccctcgtggtaagatagacccatctcctcgaataaatgtattggtggtgatttggaaaaaaaaaaaaaaaaaa

SEQ ID NO: 234 - DNA, 786 bp, Mus musculus
agctccgcccctgctactggaccatggaractgtggcccagtagagaccttagtgtgaggctttcaggggcggcggccatggaggccgtgctgaacgagctggtgtctgtggaggatctgaagaattttgaaaggaaatttcagtctgagcaggcagctggttctgtgtccaagagcacgcaatttgaatatgcctggtgcctggttcgaagcaaatacaatgaggacatccgcagaggcatcgtgctgctggaggagctgttgcccaaagggagcaaagaggaacagcgggactatgtcttctacctggccgtgggcaactaccggctcaaggaatatgaaaaggctctaaagtatgtgcgagggctgttgcagactgagccccagaacaaccaggccaaggagctggaacgcctgattgataaggccatgaagaaagatggactggtaggcatggccatcgttggtggcatggccctgggcgtggcaggcctggctggactcattggactggctgtctccaagtccaaatcctgaaggcagcctcacctgctctctgccccgggacgcctaggagcctgggggacactggaagaggggcctgtccatcctcaccatcgccttcccttttctcctgcacccctgtagtctacctctacagtctccatgacccccagcctyttagcccctgcacctgtcgtttaaccctgtcatgctttgcaatgagtgtaaataaaattgggccgtggctcgggaaaaaaaaaaaaaaaaaaaaaaaaaaaaaaa

SEQ ID NO: 235 - DNA, 980 bp, Mus musculus
  misc_feature (847)..(847): n = any nucleotide
agattgacttgggcactgacatggttcctgccatctccctggcctacgagcaagctgagagcgacatcatgaagaggcagcccagaaaccccaaaacggacaaacttgtgaacgagcgtctgatcagcatggcctatggacagatcggtatgatccaggccctgggaggcttcttcacttactttgtgattctggctgagaacggtttcctgccctttcacctgttgggcatccgagagacctgggatgaccgctgggtcaacgatgtggaggacagctacgggcagcagtggacctacgagcagaggaagatcgtggagttcacctgccatacagcgttctttgtcagtattgtggtagtgcagtgggccgacttggtcatctgcaagaccagaaggaattctgtcttccagcagggaatgaagaacaagatcttgatatttggcctctttgaagagacagcccttgctgctttcttatcctactgccccgggatgggggcagcccttaggatgtatcccctcaaacctacatggtggktctgtgccttyccctactcccttctyatctttgtgtatgacgaggtgcggaagctyattatyaggcggsgccctggcggctgggtggagaaggagacctactactagcccyactgccctgsacgccgkggaacattgtgccacacactgcacccaccccwaccccctctwysysymcttyaagtyttggagctcggractctwccctsrwaggrwwkywssrargcmtktgggratkywkacsycytggratgragsawrtagctgyamcssrrrrcsgkkygkkggggaggrytgcctgagaaacnccgtgtmckkggacgacakcggrmaagrattkwtacgtgcctttttgtttttgtaaaaaaggaaaacctggaaagactgaaagattacgttttatatctggatttttacaaataaagatggctattataacgg

SEQ ID NO: 236 - DNA, 718 bp, Mus musculus
  misc_feature (642)..(653): n = any nucleotide
agccggaggccgagcccagtcgccagctcctgctctgctcctctcccgcctgccgccgcgctgcacgcctcgagcactccctcggccccggcggggaccggggaccccgcagctaccgccatgctgccagtgctctacaccggcctggcggggctgctgctgctgcctctgctgctcacctgctgctgcccctacctcctccaagatgtgcggtacttcctgcggctggccaacatggcccggcgggtgcgcagctaccggcagcggcgacccgtgcgtaccatcctgcgggccttcctggaacaagcgcgcaagaccccacacaagcccttcctgctgttccgagacgagacgctcacctacgcccaggtggaccggcgcagcaaccaagtggcgcgggcgctgcacgatcaactgggcctacgacagggggattgcgtagccctcttcatgggcaatgagccggcctacgtgtggatctggctgggactgctcaaactgggctgtcccatggcgtgcctcaactacaacattcgtgccaagtctctgctgcactgctttcaatgctgcggggcgaaggtgctgctggcctccccagatctacaagaagctgtggaggaggttcttccaaccctgaaaaangatgccgtgtnccgtcttttacgtaagcagaacttctaacacaaatggtgtggacacaatactggacaaaagtaga

SEQ ID NO: 237 - DNA, 601 bp, Mus musculus
tttttttttttttttagtgttaatatagtttattatgtctttaaaaaaataaggccctctctccaagaagcttagtttgcaaggacaaatggcaggtgcacattgaaaaataattgtttctaaatctttttacttgcaaaggttcaggtgtaatttaaaaaaaaaacaaacaaactatccttttaatgaataatttcctaaaaataaaatcgcaccttatagccttcaatcaagttaaagttggattctacgtatgaagtggctctgcgaggtctatcgagtttctttctggaaatgtcatgagctaaaccaccagggaatattcagagcttcagagttttatcaattatggcattataaatgttctcagtcatgggcacaaatgttttctctgcatcatccatgaaatacaaggtatctttgatgactgtgggattgaagccctcttccatcagggtcactttgcggtgtttaaaagtcccagtgatctcaatggtatcttgtatcctcaggaaccgaggcctcgcgtaactgggcaggtactccgcgatgtgttgaaagagtttctttccattgaactcgtagttttctttgatcttga

SEQ ID NO: 238 - DNA, 1302 bp, Mus musculus
  misc_feature (880)..(880): n = any nucleotide
agcgcgggaggcgcatggcgggcatggcgctggcgcgagcatggaagcagatgtcctggttctactaccagtacctgctggtcactgcgctctacatgctggagccctgggagcgaaccgtgttcaattcgatgctggtttccgtggtggggatggccctgtacactggctacgtcttcatgccccagcacatcatggctattctgcattactttgaaattgtacagtgacgaagatgtgaccaggatccagaggttcctggggaagatctgccttgtgaagttggaatgagacctcatcagatgtaagatgtgctacggatgtccacgtgaccaaccttataaatacaaagactttaaaaaaaacttcatgagtagaacaggaaaaatcatcctggctcatgtgttgwgttctttctttttgattttaacagaggctcttatatagtagcttttatctattttaacattgtagtcatttgtactttgatatcagtattttcttaacctttgtgactgtttcaatattatccagtgaaagcttttcttaatgtaactttgagtacatctcaattgccttctatttttaaaacctaaggtcattagttgggctttactgttcttgctatcatatggcatatacatctgcctggatatatttctactcttgaccaaagttttgtaagaaacaatacagaatttggggacgggagggaggatactttgagaactacttacaaaagatttatctgcttgcttgaactcaggagtacggttttagctccctagactcttaacagcttttgctctaaaactattaaagtgtttcttagtaatgaaaaagtaaagatcttgctaacattaatacaaggaacatttcnccttttagatattaaattcttatgtggacttatttccaaccaactttgatgattcctgtgattggtgattggtggccggcttgtatgacatatgctgagatacactgtggagcttttcatagaaggcttatcagatcctctgaggcagtattgaccctgtatacgctcaagagttaatttgatgttgtaacaggcgttgtgaaatgcatttgcacgttgtgataaagacmcaatgagatgaattgtgcagaaccataaccaaaagaaatgtaaacttgcttaaaaatcctttcactctttgtattttttttaaaacggtttttattccttagatgtaaaatgactacttttttttgatgtaaactcattaaattcaaagaaaaatgtaaaaaaaaaaaaaaaaaaaaaaaaaaaaaaaaaaa

SEQ ID NO: 239 - DNA, 1362 bp, Mus musculus
  misc_feature (39)..(1293): n = any nucleotide
agcgagtttgcagacttcttgtgcgcagctagccgcctnaggtgttngaaccatgaatcttttactcctttnggctgtcctctgcttgggaacagccttagctactccaaaatttgatnaaacctttagtgcanagtggcaccagtggaagtcnacgcacagaagactgtatggcacgaatgaggaanagtggagganagcgatatgggagaagaacatgagaatgatccagctacacaacggggaatacagcaacgggcagnacggcttttccatggagatgaacgcctttggtgacatgaccaatgaggaattcaggcaggnggngaatggctatcgccaccagaagcacaagaaggggaggctttttcaggaaccgctgatgcttaagatccccaagtctgnggactggagagaaaagggttgngtgactcctgtgaagaaccagggccagngcgggtctngnngggcgtttagcgcatcgggttgcctagaaggacagatgttccttaagaccggcaaactgatctnactgagtnaacagaaccttgtggactgttctcacgctcaaggcaatcagggctgtaacggaggcctgatggattttgctttccagtacattaaggaaaatggaggtctgnactcggaggagtcttacccctatgaagcaaaggacnggwtcttgtaaatacagagccgagttcsctgtggctaatgacacagggttccgtggatatccctyagcmaggagaaagccctcatgaaggctgtgcgactgtggggcctatttctgttgctatggacgcaagccatccgtctctccagttctatagttcaggcatctactatnaacccnactgtagcagcaagatcctcgaccatggggttctgttgntnggctatggctatnaaggancagattcaaataagaataaatattggcttgtcaagancagctggggaagtgaatggggtatggaaggctacatcnaaatagccnaagaccgggncnacctctgtggacttgccnccgcgnccanctatcctgtcntnaattnatnggtagcgntaatgaggacttatggnctctatgtccnaaggaattcagcttaaaactgaccnaacccttattnantcnaaccntggtncttnaatcattnaggatccaantcatnatttnaattctgttgccatttttacatgggttaaatgttaccnctacttaaaactcctnttataaacanctttataatattgaaaacttagtgcttaattctnagtctnnaatatttnttttatataaaggttgtataaaactttctttacctcttaaaaataaattttagctcagtgtgtgtgtaa

SEQ ID NO: 240 - DNA, 471 bp, Mus musculus
  misc_feature (388)..(468): n = any nucleotide
gagcgccgccgggtcggccagcgaggcctcagaggttccagacaacgtgggagactggctccgcggcgwcttccgcttcgccaccgatcgaaacgacttccggaggaacttgatcctyaatttgggactctttgctgcgggagtctggctggccaggaacttgagtgacattgatttgatggcccctcagccaggggtgtagccagagaatggaactcctgtgtattcagactttccaaagacagcctactgtctgtgaccacaagatcctacctgagtggcagctgaagttgactccctctccktgcctgaacccccccccwctgcccccccatcccccagtgtcggctragatgttgcctctgcacggttctgtgtgcagttcccnactttctgcagaagatggtccttgcccttgtcctnaagagtaataatggttcttgaaaaagatttcaaataaagcctgcncat

SEQ ID NO: 241 - DNA, 636 bp, Mus musculus
aaaggaaaaacacagctcagcagatccaggcactaaagagagctagctgcaagcaggagcagtcaagaatctgtggtcagaagtactggagtgggccagcagggccagctttttctaccatggcagcccaaggctacggctactatcgcactgtcatatttgcggccatgtttggaggctacagcctgtactatttcaaccgcaaaaccttctcctttgtcatgccctccttggtggatgagatcgctctggacaaggacgatttggggctcatcacaagcagccagtcggcagcctacgccatcagcaagtttgtgagcggggttctgtcagatcagatgagcgcccgctggctcttctcctctgggctgctcctggttggtctggtcaacgtagtcttctyatggagctccacagtgtcagccttwgctgctctttggtttcttaatggtctggcacaggggctgggctggcccccctgtgggaagatcctgaggaagtggtttgagccatcccagtttggcacttggtgggctgtgttgtcaaccagcatgaacctggctggaagtttgggacctatcttggcaacgatcctcgcccagagctacagctggcgcagcacactggccctgtctg

SEQ ID NO: 242 - DNA, 381 bp, Mus musculus
ttttttttttttttttttttttttttttttttttttttttttttttttagdaacaaatctagctttattgatctatacaagkcggtttgccctctatattcttcccttttgkcccagggtattcatgagagggggggacycctytyctcccttagggactgrggtattggcaacaattttgccccctgccaagaagccttggggtacagcagaaaccacaggccattaatactcactaggagatcaggacctaggagcaagaagaagggtataggagacactctgaattcaggagttgcctgtctgccagaaggkagaaacagaagctgawggagtctagaaccaaacatcatcattttaaatagagcatgggaagggagt

SEQ ID NO: 243 - DNA, 443 bp, Mus musculus
agcggaactcaagatggctgcgctcttgctgagtgctgctcctttgggaaccacagctaaggaggagatggagcggttctggaagaagaacacgagttcaaaccgtcctctgtctccccatttgactatctacaaatggtctcttcctatggcactgtccgtttgccaccgaggctctggaatagccttgagtggaggggtctctctttttggcctgtcggcactggtgcttcctgggaactttgagtcgtatttgatgtttgtgaagtccctgtgtttggggccaacactgatctactcggctaagtttgtgcttgtcttcccgctcatgtaccactcactgaatgggatccgacacttgctatgggacctaggaaaaggcctggcaataccccaggtctggctgtctggagtggcggtcgtggttcttgctgtgttgtcct

SEQ ID NO: 244 - DNA, 580 bp, Mus musculus
cttyttttttttttttttttttttaaaakgagtagtactttaatcgtttccaaagaagatggtacatgaatttagattagaaaatatgacattaaaggtgtattttcctagaaatgaaaaacagaacagaaacttaatacaattttttttgatcttcctcatacatcaaaagctgtgtatctgttcccaggcttgagctccaagctcccaggctgctctttatctccctctagctgagcaggacaatccctcctcctccacctctctcctctcagtcatgaggtgtgggtgctaaaggccctaagctgaccaatggacagaagtgtcctttaagccagcagcccacaggcagtggcactcggggcggtcacagaactggcattgtttmyactgtctcagcagaaagcagcggaaagctgaccctcagcacaaatcagtcaaatgtggaaccggagcaagggctcccaggcccagggaggatgaccctttcgtactacaacaggtctttaagccgctctactgcactccaaggggattgaaagcatgtggcatctaaacaaatcaggagaacctgggtggt

SEQ ID NO: 245 - DNA, 1475 bp, Mus musculus
gaggggaacaagatggcggcgccaaaggggaagctttgggtccaggcccaactggggctcccgccgctgctgctgttgactatggcgctggccggaggctcggggactgcagcggccgaagcctttgactcggtcctgggagacacagcgtcctgtcaccgggcctgtcagctgacctaccccttgcacacctacccgaaggaagaggagttatacgcatgccagagaggctgcaggctgttttcaatttgccagtttgtggatgatgggcttgatttaaatcggaccaagctggaatgtgaatctgcgtgcacagaagcatattcccaacctgatgagcagtatgcttgtcatcttggctgccaggatcagttgccatttgctgaactgagacaagaacaactcatgtccctgatgccaagaatgcatctcctcttccctctgactctggtgaggtcgttctggagtgacatgatggactctgcacagagcttcataacctcttcatggactttttatcttcaagccgatgacggaaaaatagttatattccagtctaagccagaaattcagtatgcaccgcagttggagcaggagcctacaaacttgagagaatcatctttaagcaaaatgtcctatctgcagatgagaaactcacaagcacacaggaactaccttgaagaggaagaaagcgatggctttttaagatgtctatctcttaactctggatggattttaaccacaacccttgtcctctcggtgatggtgttgctctggatctgttgtgcagctgttgctacagctgtagaacagtatgttccccctgagaagctgagtatctatggtgacttggaatttatgaatgaacaaaagctgagcagatacccagctccttctcttgtgattgttaggtctcagactgaagaacatgaggaggcagggcccctgcccaccaaggtgaaccttgctcactcagaaatctaagctttttaaaagagtcgtggacacataaacttccattcctcatagagctttttaagatggtttcattggacataggccttaagaaatcactataaaatgcaaataaagttaccaaactctgtgaagactttatttgctgtgactttacctgtatttttctagtcatttaagatggacattgggttgtatttttattttactaatatctgtagctacttagttagttgcattggttttggtttttttcctctcttcgccaaattctatgagctgatcattgtggccccgcccctgccatgccccccgtcagtcatctcacttaataaccgaaaccttagggtgtgatgcttctgcccggaaatggcctccaaactgtcctctggattatagcagaaatgttatttaatgacwctacattttcagttgtattgaattgaaatcattaaaatctatttgaataattatgttctgaaaaaaaaaaaaaaaaaaaa

SEQ ID NO: 246 - DNA, 724 bp, Mus musculus
  misc_feature (609)..(681): n = any nucleotide
aaaggtacgaagctagggaagatattcgcgtggctaaatctgcacgtggaaggagcattaacttggccctttcttatagaggacggcaggccttgaaagccattggtctggaagatcagatcgtttccaaaggtgtgcccatgaaagccagaatgatccactctctttctggaaagaagtctgcaattccctatgggaacaagtcacagtatatcctttcaataagcagagaaaacttaaacaaggacctgctgactgccgwggagtcctatgccaatgcgaaggtgcactttggccacaagctgtcgaaatgcattccggaggaaggggtactcacagwgctcggacctgacaaggttccccgagatgtcacatgtgaccttgttgtagggtgtgatggagcctattcaactgtcagagcccacctcatgaagaagccccgctttgattacactcagcaatatatccctcatggatacatggagttgacaattccacctaagaatggggagtacgccatggaacctaactgtcttcacatttggcctagaaatgcctatatgatgatcgcccttccgaacatggacaaatctttcacatgcaccttgntcatgccctttgaggagtttgaaagacttccaacgcgcagcgatgtgctggacttcttccagaagaactttncagatgctatccctctgatgggagagcaagccctcatgagaga

SEQ ID NO: 247 - DNA, 827 bp, Mus musculus
  misc_feature (8)..(191): n = any nucleotide
cccttttnnccccaattgggccgttaggcgtttccggtggaaaggtcctannttaagcagangccncttcggggctaacctanaagnnccccncttggnttnccnggccttntcggaaatttnatttcggaccncccctttaagggagnccccaagcttgggtcccaaacctcggnttcccctagttccgngcggccaattgtgctgaaagcgrctgcamagtgaagccctctgttacctgtgtcgatcaagacctcaawccccagaggaacttcgtcatcaacatgacttgcaggttttgctggcagcttcctgaaacagactacgagtgttcaaattccaccacctgcatgaccgtggcttgccctcggcagcgctatttcgccaactgcaccgtgcgtgaccacattcactgcctgggcaaccggactttccctaagctgctgtactgcaactggacaggtggctacaagtggtcgacagccctggctctcagcatcacccttggggggtttggagccgatcgcttctacctgggccagtggcgagaaggcctcggcaagctcttcagctttggcggcctgggaatatggaccctaatcgatgtcttgctgattggagttggctatgtgggaccagcggatggctctttgtacatttagccgaggttatgtgcttcagagagcagtgtagagtcctgtgtgtggagatggatgcggtgtgagagggggatttatgtttttaatgtacagcatctgtacttggcttgccttgatgaaggtaaaataaagaaatcgaacmctgaaaaaaaaaaaaaaaaaaaaaaa

SEQ ID NO: 248 - DNA, 946 bp, Mus musculus
  misc_feature (922)..(941): n = any nucleotide
cctggtaggagccgcagtgtgtggccatgcctcagataggtttgggcgcagaagggtgctgacctggagctatcttctggtgtccgtgtccggcacagyagctgccttcatgcccaccttccccctctactgcctgttwsgtttcctgctggcctctgcagtggcaggtgtcatgatgaacacagccagtctcttgatggagtggacatcagcccagggtagccctttggtgatgaccttgaacgccttgggcttcagctttgggcaggtcctgacaggttctgtggcctatggtgtgcgcagctggaggatgctacagctggctgtctctgcccccttcttcctcttctttgtttattcatggtggctaccagaatcggcacgctggctcatcacggtaggcaagctggaccagggcctgcaggaactacagagggtagctgctgtcaacaggaggaaggcagagggagacacgttgaccatggaggtgctgcggtcagctatggaggaggaaccaagcagggacaaagctggtgccagtctgggcactctactccacacacctgggctgcgccaccgaaccatcatctccatgctgtgctggtttgcctttggcttcaccttctacggcctggcccttgacctgcaagccctaggaagcaatatcttcctgctccaggcactcatcgggattgtggacttcccggtgaagacaggcagcctgctgctgatcagccgcttgggccggcgcctctgccaggtcagcttcctggtgctccccggactctgcatcctgtccaacatactggtgccccatgggatgggggtccttcgctcggccctggctgtgctggggctgggctgcctggggggcgccttcacctgcatcaccatcttttccagtgagctctttccccactgngatcagggatgactgcagngggcc

SEQ ID NO: 249 - DNA, 221 bp, Mus musculus
ttttttttttttttttttttytctttaaaaattatttatttgkgtgtatcaccgggggcgggtatgtgtccatgcggagctcagaggacaactttgtgaagtctgttgtcacatgagtttcagaaatttaggcccggaggcaggtgtctttacccgccgtcgccagccctgccctttcctcctcctcctcctcttcctcctcctcctcctctwcctcctcc

SEQ ID NO: 250 - DNA, 635 bp, Mus musculus
gaaaggggacggagaccttcagcgtggaatctatatccaagaatggaatctgtctggagatgggcccacagcctcagggcgtgctgcgggccgacctgttctcccggatgcgagctctggtggcatccattctggacttcatcgagctcttcaaccaaggcatggacttacccgcctttgagatggatatctacaggaacttgggcagtgtggacttcccacgcactgcggatggtgacctggctggcactgtgcaccctcaactgcaggaccatgactttgagccactgaggcctggtgaacccatcttcaagcttttcagcggagaagacgtactgtatgagggggactccattgtgtaccctgtgttcattaatgaggctgcctattatgagaagcacgtggcattcctgaagtctgagaagatcagggtcacagtgcctgccctgctgaggttgaccccccgctccacccagactccctaacccaaatcatacctggcctgatctcagttttcctatctgaacaatgggtcccaagcccagggtcccctatctcactctggacaccatatccctttccagacaactgatctcattatataaaatagactgtgccccaaaaaaaaaaaaaaa

SEQ ID NO: 251 - DNA, 713 bp, Mus musculus
  misc_feature (633)..(707): n = any nucleotide
agcggacgtttgtgccgggacatggccgctgcggatgccgggtccttgagctgagcgcttgctgccggagccaacctctgccgtcaaccgtccgcgcgggctgggcccaggcgccgggacggccaagatccgccgaggaagctgaggcagctatagaacgccgccgcggcgggcgcatggcgtccatcttgctcaggagctgccggggccgggggcctgcccgcctcgcgccacctcgggccgcctccccgcggggtagtctgagggatcgagcttgtctcagctgtaccaggaccctggggttgacgagccgtgagagtgttctgtctcgttgctgtactccagcccaccctgtgtacctctgcttcaaaggtgagcccctcagctgttggactcagaggcctgagtgccagggcaccgcagcaagaacaacatggacacctgcctctgcaaggctggtggttacgggacctcagtaccttcctgtgcgcggctggcactcatcatctycgctaggagaggactctgtgatagagaagtcccttaagtccttaaaagacaagaataagaagctggaggagggcggccccgtgtacagcccggccgcgcaggtggctggtgaggaaatccctngggcagaaggtactggatgagctgaggcactactaccatggctttccgcctgctctgggatngacaccaaagancgcttg

SEQ ID NO: 252 - DNA, 774 bp, Mus musculus
  misc_feature (112)..(772): n = any nucleotide
tttttttttttttttttttttttttttttttttaattatggaagtttctgatttattccagacaaaatattagatttgccactaagaatcacctcaaagcaatcactcttgngtgtcatgcccctgnggctggcacggcatgatgaaggcagacctgngggccagaagctggcagctctaattcttcacttctgcggcctctttctcagccttctccttggccttctctttctcttctatcttttcttctttctctagngnggccacaatttcagctacctgggnggnagagatctgaacatcttctttgttcaccaagtcgattaccttgacaaggtcatcgatgttgatattgccgnccttattatcatccagggctgaggtcaaactgatgagcttgagctctggaatgngcttgatttgcttcatggcgctgatgagctcagtgatgctgatgacactctcccctgtwggagngctctggctggggcccagcttgccatcctgwtgagaggwctccagctgtgtgatcaggccatcgatctgcccgatcatctgctgcactcgctttgacagcctcttgctcgctgcarawtcttctatgnactttctcctcacccgtctnttgaaagktccttctttgawctcctgtaagtcctcacntgtaggcctggacgccctcttttgagmrsrsntccaggcttcctcctttctcccttggtcagggacttcttctgctnctgcagcttggaacaggcgtactgaggatgtnaa

SEQ ID NO: 253 - DNA, 808 bp, Mus musculus
  misc_feature (794)..(807): n = any nucleotide
atggaccgcttcgtgtggaccagcggcctcctggagatcaacgagaccctggttatccagcagcgcggggtgcgcgtctacgacggcgaggagaagataaaatttgatgccgggactcttcttcttagtacacaccggctgatttggagagaccagaagaataatgagtgctgcatggccattcccctgtctcagattgtgttcatcgaggagcaggcagctggaatcgggaagagcgccaaaatcgtggttcacctgcacccagctccttctaacaaagagcccggtccgttccagagcagtaagaactcctacatcagactctccttcaaagaacatggccagattgagttttacaggcgtttatcagaagaaatgacacagagaagatgggagactgtgccagtttcccagtcactacaaacaaataaaggaccccagccaggaagagtaagagctgttggaattgtaggtattgaaaggaaacttgaagaaaaaagaaaagaaaccgataaaaacatttctgaggcctttgaagacctgagcaagctgatgattaaggctaaggaaatggtggagttatccaaatcaatcgctaataagattaaagagaagcaaggcgatgtcacagaggatgagaccatcaggtttaagtcgtacctgctgagtatgggaatagccaacccagtcaccagagaaacctatggctccggcacacagtaccacatgcagctggccaaacaggctggctggaatattgcaggcacctttggaggagcgtgggggaataatgnccctcacagaggng

SEQ ID NO: 254 - DNA, 722 bp, Mus musculus
  misc_feature (654)..(654): n = any nucleotide
aaagtggggatcctggwgacagttctggtgcagagttccagtacctctacatccatcattgtcagcatggtctcctccggcttgttggaggtgagctccgccattccgatcatcatgggctccaacatcggaacctctgtcaccaacaccattgtggccctgatgcaggcaggggacaggactgacttcaggcgggcttttgcaggggcgaccgtgcatgactgttttaactggctgtctgttctggtcctactgcccctggaggctgccacgggctacctacaccatgtcaccgggcttgaggttgcttccttcaacatccgaggtggccgtgatgcccccgaccttctcaaagtcatcacagagcccttcacaagactcatcatccagctggacaagtctgtgatcaccagcattgccgtgggggatgagtccctgaggaatcacagtctcattcggatttggtgtcacccagacacaacagaggcttccacttctatgtccagggtagaggccatcggcagccttgcaaacaccaccatggagaaatgcaaccatatcttcgtggatacggggctgcccgacctggcccgtggggctcattctgctggcgggctccctggtggttctctgcacttgtcttatcctccttgngaagatgctcaactccctgctcaagggccaagtggccaatgtcatccagaaggtcatcaacacagact

SEQ ID NO: 255 - DNA, 779 bp, Mus musculus
  misc_feature (608)..(695): n = any nucleotide
agggcgccagctgaagacgcgggacttaaagcgcgtagccagaacccaggcaccagtgtgtccattgtccagaactcatctgaaaaactgccacaggaattgcttctctgctccaggctggtcactgaacaggttgctccaggacctgcagaatgggggcaggctgtgtcaaagtcaccaagtatttcctcttcctcttcaacttgctgttctttatcctgggtgctgagatcctgggcttcggggwgtggattcttgcagacaagaacagcttcatttccgtcctacaaacctcatccagctcgctgcaggtgggggcttacgtcttcatcggwgtgggcgccatcaccatagtgatgggcttcctgggctgtatcggwgctgtcaatgaggtccgctgcttgctgggtctgtactttgtcttccttctgctgatcctcatcgcacaggtgaccgtaggggtcctcttctacttcaacgctgacaagctgaagaaggagatggggaacacagtgatggacatcattcgcaactacactgccaatgccaccagtagccgcgaggaggcctgggactacgtgcaggcgcaggtcaagtgctgtggctgngtcagccactacaactgnacagagaacgaggagctcatgggctttaccaagaccacttacccatgctcctgcgagaaggatcaaggnaagaggacaaccagctcattgtgaagaaaggattcttgcgaggctgataacagcacctgtgagcgaaaacaacccctgaggatt

SEQ ID NO: 256 - DNA, 655 bp, Mus musculus
  misc_feature (641)..(653): n = any nucleotide
aggagccctcgaaagcgacatggcggttctcttaaagctgggcgttctctgcagtggccaaggagctcgagctctcctactccgaagccgggtggtcagacccgcttatgtgtcagcatttctccaggaccagcctacccaaggacggwgtggtacccagcacattcacctgtcaccaagccaccactctggttycaaggctgcatctctccactggaccagtgagagggwwgtcagtgttctgctcttggggctgatccctgctgggtacttgaatccctgctctgtggtggactactctctggcttcagccctcaccctgcacagtcactggggccttggacaagtggttaccgactacgttcatggggacaccctgccgaaggctgccagggcaggcctcttggcactctcagctttgacctttgctgggctttgctacttcaattaccacgatgtcggcatctgcagagcggttgccatgctgtggaagctctgacctgggtgcagcactttgattgtgtgcctccttgcctctgctttaccaatgccgttcacctcgcagtgaggggggatgaaggataagcccattggtgggcagaatgtcttctaattacatgggtattttcagaattatttgntgaggaagaggntt

SEQ ID NO: 257 - DNA, 516 bp, Mus musculus
tttttttttttttttttctttttttaagataatggtcttttaattgagttattgagatgaagagacagtgaagccctgtttgctacttacatgaaaagaagattttaaaaaacaatcactgcacaaaatacaaaggggcagggtatgctgwggcattgaattcctccccacgttttttcttgacttctcaagaacaaattaaagtctccacagcaaatttgttctcaaaatgccgaatggtgaaacagttactggcttcccgcttctgaatacctctaattgtttcccggcgctgcagtttgtaggtctccttgtcgtgacacagtcggtagatgaagaagcccaggttgtccacgttctcgatgcggtcggtgatcaccatgtgctcatggataaggtaggactgaggcaggtaggtcccggccttaatgttaataaggagctccagcaggtttttgggcggcataacgatggaagtgttcagaggaatcacgtagcacttgtccaggttaaggt

SEQ ID NO: 258 - DNA, 788 bp, Mus musculus
  misc_feature (701)..(787): n = any nucleotide
aaagagcggctgctgtcggaagcaccgggcgagctatctgttacagtccggcccggggatggctcgggacgcggagctggcgcgcagtagcgggtggccgtggcggwggctgccggcgctgctgctgctgcagctgctgcggtggaggtgcgccctgtgcgcgctccccttcaccagcagtcggcacccaggctttgcggacctgctgtcggagcagcagctgttggaggtgcaggacttgaccctgtctttgctgcagggcggaggtctagggccgctgtcactgctacctccggacctgccggatctggagcctgagtgccgggagctgctgatggacttcgccaatagcagcgccgagctgaccgcctgtatggtgcgcagcgctcggcccgtgcgcctctgccagacctgctacccgctcttccaacaggtcgcaatcaagatggacaacatcagcccgaaacatcgggaatacctccgagggcccgcgctgaggcggaagtctcctgacggcagacagaatgcagatagttctcatggtctctgagtttttcaacagcacgtggcaggaggcgaactgcgcaaattgcctaacaaacaatggtgaggatttgtcaaacaacacagaggacttcctcagtctgtttaacaagactttggcctgctttgagcataacctgcaggggcacacatacagnctccttccaccaaaaaattactccgaagtgtgcagaaacttgtaaagaggcatataaaaacctgagcctnctgtacagtcaaatgna

SEQ ID NO: 259 - DNA, 617 bp, Mus musculus
  misc_feature (300)..(613): n = any nucleotide
ttttttttttttttttttttttttttttttttttccttgttatttcctttattggaaagcataaggaaaaaacaggttttcttgdgcacacacataaccctatgkgcctmaggattcagaactatgtacatttttttaatatgaccacaagatgaaattctttggcacattttcaaatatatttctaatgcaacctctagagagccagaccctgatcaggaacagagatggctggcttgttaaagggctctccagcttcttagccaaaagcaggggtttgtacacacagtactgaaaggnacccgaggaagtcgctactcacaggtttaaatatgtcacttcactcactttgcacatgtaaataagntttacatgtactgatgaagatggnttccaatgaccctnaaccatgngcttcaaatcaagacaggaacaatgacagcncaatgaaccccggcacatntaggggatcacagcgncgnctgattgtcacatacccggggtgacacactctgggactaagactagcctgctntcacactctgcanatgtggnaaacatacaaaaaatacccaaacactcctgccttcctgtagggcaaanacaggttttnaagg

SEQ ID NO: 260 - DNA, 720 bp, Mus musculus
aaaggcgcggagcctgctgctgccatggtggctggtggctgggtacactatgtctagactgggggccctgggcggctcccgcgccgggttgggactgttacttggtactgccgccggccttggattcctgtgcgtcctttacagccagcgatggaaacggacccagcgccatggccggagtcacagtctgcccaactccctggactatgcgcaggcttcagagcgtggacgccaggtgacacagtttcgggctatcccaggtgaagctggagatgctgccatactgcccagcctctcacaggaagggcaggagaaggtgctggaccgcctggactttgtgctgaccagtcttatggcgctgcggcgcgaggtggaggagcttcagagaagcctgcaaggactagctggggagattgtcggggaggtccgctctcatatagaagagaaccagagagtggcccggcggcgcaggttcccttttgccagagagaggagtgactccacgggctccagctctgtctacttcaccgcctcctcaggggccgcactcacagacgccgagagcgagggaggctatacaacagccaacgcggagtctgattacgagcgggactccgacaaggagagtggagatgctgaggacgaagtgagctgcgagaccgtgaggatggggaggaaggactctctggacctggatgtggaggcggcgtccagtccgtc

SEQ ID NO: 261 - DNA, 735 bp, Mus musculus
  misc_feature (672)..(703): n = any nucleotide
aggcttgccgtccaggccatgcggctggwggtctgcatcgwggcgtttcccatgtttctgctgaaccttctaggcatgtggagctgggtatgcaaaaagtgctttccctacttcctgaagcggttcgccatgatatacaattggaagatggcgagcctaaagcgggagctcttcagcaatctgcaggagttcgccggcccctcggggaagctaactctgctggaggtgggctgcggcaccggggccaacttcaagttctatccccccgggwgcagggtcacttgtatcgacccyaaccccaactttgagaagttcttgttcaagagcgtcgcagagaaccggcagctgcagttcgagcgcttcgtggtggcagccggggaggacatgcaccaggtgaccgatggctctgtggacgtggtggtctgcaccctggtgctgtgctcggtgaagaaccaggagaagattctgcgcgaggtgtgccgagtgctgaagccgggaggggctttttacttcatagaccatgtggcagatgaacggtctacctggaattacttctggcagcaggtcctggctcgtgtgtggttccttgcttttgacggatgcaatctgaccagagagagctggaaggccatagagcaggccaacttctccaagctgaacctgcagcacatncaggccccgctccccttgaccttggtgcggncccacatctatggctatgctgtgaaatagagt

SEQ ID NO: 262 - DNA, 528 bp, Mus musculus
  misc_feature (502)..(502): n = any nucleotide
ttttttttttttttttttttttttttttttttttttttggcaaatatagaaaccatttatcaaatgaatataaatgtattgatcaacatttaaaatataacttctgcaaaatcatcttgaaaaatatacatttgtttagatccatacatacaaatgcagctgaaacccttgggcccacccagacttgctctctgtatgaacacaatgatatccatggttttgtttcaggaccagkggaatttttcttcttcttcaatacagggtttatttgtgtagccctggcggkcctggaactccatctgtagatcaggctgtactcacagagatccacctacctctgccacctgagagctgggattgaggctctgccaccaaccactcaggaccagtgatatttgaccagaagaatccctccccccccgsaccccgagtccttggtaactactctcgggcattacttttagggkcccctacatactggacccatttccctaactatagnggccttctactgcctacggtaattat

SEQ ID NO: 263 - DNA, 1163 bp, Mus musculus
aaagaaggaactaaacatgggccagcgatgctctgacaccagaggaattgctttcgaagacgtcagagtgcctaaggaaaatgtgttaatcggtgaaggagcagktttcaagatcgcaatgggtgcttttgatagaaccagacctacagtcgcagctggcgctgtcgggctagcccagagagctctggacgaagccacgaagtatgccctggataggaagacatttggaaagctgctagtggagcaccaaggagtttcatttctgctcgcagaaatggcgatgaaggttgaactcgctaggctcagttaccagagagcagcctgggaggttgactccggtcgccggaacacttactatgcctcgattgcaaaggcctttgctggagacattgccaatcagctagccactgacgccgtgcagattttcggaggctatggattcaacacagagtaccctgtggagaagctgatgagggacgccaagatctatcagatttatgaaggtactgcacaaattcagaggctgatcatagctcgtgagcacattgaaaagtataaaaattaacaggaattactattgaacgatgcatcaccctcgtgtaactaagctccaagcactgttgctgcttcaggggaaaagggctttactgtcttcccaaggaaatgagataaaagacgagtttggatctatgcggcggattcccatggcggaggaacctgtcttcagctctatggtgaccctttctagataggtttggcttttggacaatgattggtccttagccccgaattgtgttagtttgctctttgatcacttaaaatggaaaaacaccctggacttttaatgttcattcaagtgacaggaaaggcggcttgtcaaggaagaactcatgattctaacataaacactgaaaatttgtggtagattggacacgtcagactgtgacatagcagcatttctgtgctgaactgttaattttataattttgattatatttgctttgttttgcacaaaagagtaaaaagtttatattcatattctcccattataaaactaaaacttttctgggaatcttagtactgaacgagaattttatttgtcttgtttaaaataattcaataaagctaaccttaacttaaaaaaaaaaaaaaaaaaaaaaaaa

SEQ ID NO: 264 - DNA, 720 bp, Mus musculus
agcgtcgccatccgccaccatggtgaacttcacagtagatcagatccgtgccatcatggacaagaaagccaacatcgataagggtgaggtgtctgcccgccaggagctcaaggcacgtgcccgctacctggccgaaaagtatgagtgggacgttgctgaagcccgcaagatctggtgctttggccctgatggcactggccccaacattctcaccgacatcaccaagggtgtgcagtacctgaatgagatcaaggacagtgtggtggctggcttccagtgggctactaaggagggcgctctctgtgaggaaaacatgcgtggtgtgcggtttgatgttcatgatgtgaccctgcatgctgatgccattcaccggggaggtggccagatcatccccacagcacgccgctgcctgtatgccagtgtgctgaccgcacagccccgcctcatggagcctatctatctggtggagatccagtgtcctgagcaagtggtgggtggcatctacggtgtcctgaacaggaagcgtggccatgtgtttgaggagtcccaggtggctggtacccccatgtttgtggtcaaggcatacctgcctgtcaatgagtcctttggcttcaccgctgatctgcgatccaacaccggcggccaggccttcccccagtgcgtgtttgaccactggcagatcctgcctggggatccttttgacaacagcagccgccccag

SEQ ID NO: 265 - DNA, 729 bp, Mus musculus
aaaggccagctcctgctctgctcctctcccgcctgccgccgcgctgcacgcctcgagcactccctcggccccggcggggaccggggaccccgcagctaccgccatgctgccagtgctctacaccggcctggcggggctgctgctgctgcctctgctgctcacctgctgctgcccctacctcctccaagatgtgcggtacttcctgcggctggccaacatggcccggcgggtgcgcagctaccggcagcggcgacccgtgcgtaccatcctgcgggccttcctggaacaagcgcgcaagaccccacacaagcccttcctgctgttccgagacgagacgctcacctacgcccaggwggaccggcgcagcaaccaagtggcgcgggcgctgcacgatcaactgggcctacgacagggggattgcgtagccctcttcatgggcaatgagccggcctacgtgtggatctggctgggactgctcaaactgggctgtcccatggcgtgcctcaactacaacattcgtgccaagtctctgctgcactgctttcaatgctgcggggcgaaggtgctgctggcctccccagatctacaagaagctgtggaggaggttcttccaaccctgaaaaaggatgccgtgtccgtcttttacgtaagcagaacttctaacacaaatggtgtggacacaatactggacaaagtagacggagtgtcggcggaacccaccccggagtc

SEQ ID NO: 266 - DNA, 757 bp, Mus musculus
  misc_feature (711)..(749): n = any nucleotide
cttttttttttttttttttttagtgttaatatagtttattatgtctttaaaaaaataaggccctctctccaagaagcttagtttgcaaggacaaatggcaggtgcacattgaaaaataattgtttctaaatctttttacttgcaaaggttcaggtgtaatttaaaaaaaaaacaaacaaactatccttttaatgaataatttcctaaaaataaaatcgcaccttatagccttcaatcaagttaaagttggattctacgtatgaagtggctctgcgaggtctatcgagtttctttctggaaatgtcatgagctaaaccaccagggaatattcagagcttcagagttttatcaattatggcattataaatgttctcagtcatgggcacaaatgttttctctgcatcatccatgaaatacaaggtatctttgatgactgtgggattgaagccctcttccatcagggtcactttgcggtgtttaaaagtcccagtgatctcaatggtatcttgtatcctcaggaaccgaggcctcgcgtaactgggcaggtactccgcgatgtgttgaaagagtttctttccattgaactcgtagttttctttgatcttgagggaggccatcccaattcgaccctcatgacctggcacaggcacgccatacacattcacttcttcaacaaaatctaccagtcccacgatgtcagcgaccttctgnggtagctacgntctcttcttttcaccggaaagtatctncaaccctg

SEQ ID NO: 267 - DNA, 624 bp, Mus musculus
aaaaggtggaatctatatccaagaatggaatctgtctggagatgggcccrcagcctcagggcgtgctgcgggccgacctgttctcccggatgcgagctctggtggcatccattctggacttcatcgagctcttcaaccaaggcatggacttacccgcctttgagatggatatctacaggaacttgggcagtgtggacttcccacgcactgcggatggtgacctggctggcwctgtgcaccctcaactgcaggaccatgactttgagccactgaggcctggtgaacccatcttcaagcttttcagcggagaagacgtactgtatgagggggactccattgtgtaccctgtgttcattaatgaggctgcctattatgagaagcacgtggcwttcctgaagtctgagaagatcagggtcacagtgcctgccctgctgaggttgaccccccgctccacccagactccctaacccaaatcatacctggcctgatctcagttttcctatctgaacaatgggtcccaagcccagggtcccctatctcactctggacaccatatccctttccagacaactgatctcattatataaaatagactgtgccccaaaaaaaaaaaaaaaaaaaaaa

SEQ ID NO: 268 - DNA, 698 bp, Mus musculus
  misc_feature (642)..(695): n = any nucleotide
agctgcatcgtggcgtttcccatgtttctgctgaaccttctaggcatgtggagctgggtatgcaaaaagtgctttccctacttcctgaagcggttcgccatgatatacaattggaagatggcgagcctaaagcgggagctcttcagcaatctgcaggagttcgccggcccctcggggaagctaactctgctggaggtgggctgcggcaccggggccaacttcaagttctatccccccgggtgcagggtcacttgtatcgaccccaaccccaactttgagaagttcttgttcaagagcgtcgcagagaaccggcagctgcagttcgagcgcttcgtggtggcagccggggaggacatgcaccaggtgaccgatggctctgtggacgtggtggtctgcaccctggtgctgtgctcggtgaagaaccaggagaagattctgcgcgaggtgtgccgagtgctgaagccgggaggggctttttacttcatagaccatgtggcagatgaacggtctacctggaattacttctggcagcaggtcctggctcgtgtgtggttccttgcttttgacggatgcaatctgaccagagagagctggaaggccatagagcaggccaacttctccaagctgaacctgcagcacatncaggccccgctccccttgaccttggtgcgggcccacatctatggctatgctgngaa

SEQ ID NO: 269 - DNA, 394 bp, Mus musculus
tttttttttttttttttttttttggcaaatatagaaaccatttatcaaatgaatataaatgtattgatcaacatttaaaatataacttctgcaaaatcatcttgaaaaatatacatttgtttagatccatacatacaaatgcagctgaaacccttgggcccacccagacttgctctctgtatgaacacaatgatatccatggttttgtttcaggaccagtggaatttttcttcttcttcaatacagggtttatttgtgtagccctggcggtcctggaactccatctgtagatcaggctgtactcacagagatccacctacctctgccacctgagagctgggattgaggctctgccaccaaccactcaggaccagtgatatttgaccagaagaat

SEQ ID NO: 270 - DNA, 1136 bp, Mus musculus
aaaggtggagctgggtggtgtttggtgcggtacggcggccactcagttgcagcagagcaggtgccatcctgtggaagaaccatgaagcactacgaggtggagattcgggatgcaaagacgagggagaagctgtgcttcctggacaaggtagagcctcaggccaccatttctgaaatcaagacccttttcaccaagacacacccgcagtggtatcctgcccgccagtccctccgcctggaccccaaggggaagtccctgaaagatgaagatgtcttacagaagcttcctgtgggcaccacagccacactctacttccgggacctcggggcccagatcagctgggtgacggtcttcctgacggagtatgccgggccccttttcatctacctgctcttctacttccgggtacccttcatttatggccgcaaatacgactttacgtccagtcggcatacggtggtgcacctcgcctgcatgtgccactcgttccactacatcaagcgcctgctggagactctcttcgtgcaccgattctctcacggaaccatgcctttgcgaaacatcttcaaaaactgcacctactattggggctttgctgcatggatggcttattacatcaaccaccctctctacacaccccctacctatggagttcagcaggttaagctggcactggccgtttttgtgatctgccagcttgggaacttctccatccacatggctcttcgggaccttcggcctgctgggtcgaaaaccaggaagatcccataccccaccaagaaccccttcacctggctgktcctgttggtgtcctgtcccaactacacttatgaggtgggctcctggattggctttgcsatcttgactcagtgtgtcccagtggccctcttctccctggtgggcttcacccagatgactatctgggccaagggcaaacaccgcagctacctgaaggagttccgcgactacccgcccctgcgcatgcccattattcccttcctgctctgagcagctcctcacggctctgcccagtaatactctccacccctcactgcccctgtcctgatgtgtggctggccatggctctccagcagcaacaataaaacctgcttacccaaaaaaaaaaaaaaaaaaaa

SEQ ID NO: 271 - DNA, 568 bp, Mus musculus
  misc_feature (444)..(567): n = any nucleotide
aaagcgctggagctacagccgttactgccgccgccgccgccgccgccgccgaggcgtttgatcgttggcaatgtcaggctttgataacttaaacagcggtttctaccagacgagttacagcatcgacgagcaatctcagcagtcctatgactatggaggaagtggaggaccctacagcaagcagtatgctggctgwgactactcgcagcaaggccgatttgtccctccagacatgatgcagccacagcagacatacactgggcagatttaccagccaactcaggcctatcctccaacaacacctcagccattctatggagacagctttgaggaggagccccctctgttagaagagttgggtatcaattttgaccacatttggcaaaaaacactaacggwgctacacccccctgagggcagctgacggcagcatcatgaatgaknacggacttggcagggccagtggtgttctgccttgcctttggggccacactgttactggctggcaaaatccagtttggctacgtatacgggatcagtgcgattggatgtttaggaatgntttgnt

SEQ ID NO: 272 - DNA, 587 bp, Mus musculus
  misc_feature (552)..(552): n = any nucleotide
ctctcctttgccctgcgcacgggtacacggcacggtgtggacactcacctgttcagtgtggagtcgccgcaagagctggcagcctggacccgacagttggwggatggctgtcatcggsctgctgaaggcgtacaagaagtgtctacagcctgcacgtggaacggccgtccctgcagcctgtctgtgcacatcgacaagggcttcaccctgtgggcagctgagcctggagcagcccgagccatgctgctccgacagcccttcgagaaacttcagatgtcatcagatgatggcacgagtctccttttcctggactttgggggtgctgaaggagagatccagctggacctgcactcgwgtcccaaaacgatggtcctttatcatccactctttcctgtccgccaaggtcacccgcttggggctcttggcctagaagctgtcagatgcaccagccctgaagaggatcgttcatcttgtggcctgacctgtccttctgctgactgcctgctcgtctgggggctgagggaagggagaggaggggaactagggcctcanagaccagcctgaggaagcagaactcagacctggga

SEQ ID NO: 273 - DNA, 739 bp, Mus musculus
  misc_feature (623)..(738): n = any nucleotide
aggctgtgcgaagcgggcacgcgcggaccctkcaccggccgcggagccgctatgggcccgccaggcccctggcgtggcgcggcgcaaggaccggctccgagccaggattgaaattccaagatggatgatcaggaccctgggggcattagcccccttcagcaaatggtggcctcaggagccggggctgtggwcacctccctcttcatgacacccctggatgtggtgaaggtccgccttcagtctcagagaccctcggcaaccagcgaattgacaactccctccagattctggagtctctcctacaccaaatcatcctccgctctacagtccccagggaagtgcctcctatactgcaatggagtcctggagcccctgtacctgtgcccaaatggtacccgctgtgccacctggtttcaggaccccacacggttcactggcaccttggatgcctttgwgaagattgtgcggcatgagggcactaggaccctgtggagcggcctcccagccaccctggtgatgaccgygccagctactgctatctacttcactgcttacgaccaactcaaggccttcctgtgtggtcagtccttgacctctgacctctacgcacccatggyggctgntgcccctcgccccgaatgggcaccgtgacagttgcagccccttgnagctcgtgcggaccaagctgcaggctcagcatgtgtcataccgtgagctggcttnctctgttaagctgcng

SEQ ID NO: 274 - DNA, 451 bp, Mus musculus
  misc_feature (310)..(310): n = any nucleotide
ttttttttttttttttttttttttcacagaagaccatcatctttagacaaggaatgaatggggccaagatactgcctggcccaagctcctcgggccaagctataacaaacatggggatacaaaaaagggcgatcatcagatttggtcttgaattcgggaggcgggagaggggcaatcctgggaagggaaacagcaggaatccaggtctgaggacagcctgggwgactggactgggagggaaggcacttggctcagtctcctgatcccacccgtgcaagagccggttgctccccgactttcagcggcccanaggctgctcctggttgagcctctggaagaagcttttgccaaactcgtaagtgctgatcatgatggcacaggaggggtgcagccttgatgatcctggggaggaaagcctgcaaagagtcccctggtgccagattcagcccgg

SEQ ID NO: 275 - DNA, 677 bp, Mus musculus
aaagcttcacttcgccctccagccgcggwggctgcagcgcaacttccagatagcggagtggcctcagctgcgagccgagcggwggcggcagcgyycctcaggacacccgcagatcaccttttccccgcgacttcgccatggctgagtgctgwgtaccggtatgcccacggccgatgtgtatccctccaccctatgctgacctcggcaaagctgccagagacattttcaacaaaggatttggcttwgggctggtgaagctggatgtgaagacgaagtcatgcagcggtgtggaattttcaacatctggctcatctaatacagacactggtaaagttagcgggaccttggagaccaagtacaaatggtgwgagtatggtctgactttcacagagaagtggaacaccgataacactctggggacagagattgcaatttgaagaccagatttgtcaaggwtwgaaacttgacttttgacacccaccttttcaccgaacacaggaaagaaaagtggtaaaatcaagtctgcttacaagagggagtgtataaacctcggctgtgatgttgactttgattttgctggacctgccatccatgggtcagctgtctttggttacgagggctggcttgctgggtaccaaatgacctttgacagtgccaagtcaaagctgacaaggagt

SEQ ID NO: 276 - DNA, 644 bp, Mus musculus
  misc_feature (530)..(641): n = any nucleotide
ttttttttttttttttcggttttcataaacgtctatttcatcattggtgggtagcacatttaacagttaaatacatttaaataatgtataggaggccgcaccacggcagcactgataaccatccaactaggaaccagccaacagtgactgtctaaatatttaaaatacagctcttgcttcatcatcctttgatgtgatcaccttctgggggaaggaaggggagcctgctggtcgcatggaaatatattaaggccaaatcttctgatattcgttcccagaggtttcttttaactggattaagcctccaattccaaggcaagcccaagtttgtggcctccagcattaaagctcttcccgtctaccagagcagacagtgtaagcttcacaccaggcctcagagtctgagtatagcccactccaattaaactagagttgttgacctttgcagagatagaagcagtaggatccaactggtatttagctgcaatgccaaaacgagtgcagctggtaccctgatgtccaagcgaggnttactgaagtgtcaaaatcttcacatactttctgataaatttgatcctccaaattctgtcccattatttacatttgtgtgnagctggaagtccccagtcctgtagccgacngca

SEQ ID NO: 277 - DNA, 703 bp, Mus musculus
  misc_feature (702)..(702): n = any nucleotide
tttttttttttttttttttttaagttaacaagcttgcatttaataagtctgaaaccattctcagcacatggcattgtacacgggcatctgtgcaaacagattcatttaacaggtcgtagtttaaaaaagtcatagatactgtgagttctgtataaaccggtggacggcaagttagttccttttgatttataagcctcaatgtcaccgcagaataaagaatgtagccaaagaaagcattatcggtcactcgtataggacagwgttgtttctataatttgaagctttctgaatggacgggttcaggcctgatccaactgtaaaaagatcactcagtgaatagactatatgggaactgtacaaagtgtcattaacttccatcattaatagcttactcagcactataccactattgctagttaaaataacctgcttctgaggccccacggagggaggcggcctgtgcacgcagcctcgatgccctggccaccctcatccccagggcgtgccatacagtccaacagaaaactttggctttaggaaggaatcacagacattgaaaagaatggctttaatcattattaaatgtgcagtgggaaggagtgtgcttcagatagtctgggcagggctggcggcaggcaggtcactcctgctgcacagctgcagacactagtttgtcatgacaagacaatgagggaaagcagnc

SEQ ID NO: 278 - DNA, 1098 bp, Mus musculus
aaagaagaagagggggctaagctgagtatagaggtgctccagaccagcctgcagaaggaactgactctaaacaaaggccaggcctccgccatggagctgctgcgctgccccacgcttcggcgcctcttcctctgcctctctatgctgtggtttgccactagctttgcctactacgggctggtcatggacctgcagggctttggggtcagcatgtaccttatccaggtgattttcggcgctgtggacctgcctgccaagtttgtgtgcttcctagtcatcaattccatgggccgccggcctgcacagttggcctccctgctgctggcaggcatctgtatcctagtgaatggcataataccgaggggccatacaatcattcgcacatccctggctgtactagggaaaggctgtctggcttcctctttcaactgcatcttcctgtacaccggagagctgtaccccacaatgattcggcagacgggcctgggcatgggcagcaccatggcccgggtgggcagcatagtgagcccactgataagcatgactgccgagttctacccctccatacctctcttcatcttcggcgctgtccccgtggccgccagcgctgtcactgccctgctgccagagaccttgggccagccgctgcctgatacagtgcaggacctgaagagcaggagcagaggaaagcagaagcaacagcagctggaacagcagaagcagatgataccactccaggtctcaacacaagagaagaacggactctgaaaatggagagggcgtcactcagcactaaagggagtggggttctgcaggtcccaccatctatgaggagggggagtgagtggagggactgggccatctaaatgcggagcctgccattcagagaaatgcctccccaaaggtcacttcaatagacctactaggaacaaaagctctaactgtgtgcagcttcttaagcagaatgagctcttcctcagccatcttccagctcagtggtcactcctccagctcctgtcccttccacctccaggacattgcaaagaatcccagacaattaaataaatttcttctaccttgaaaaaaaaaaaaaaaaaa

SEQ ID NO: 279 - DNA, 1356 bp, Mus musculus
  misc_feature (803)..(962): n = any nucleotide
agcaggaactgaagtctttatggccttcccactggagaccagccgcctggaaggcagatgggagcctctttttcctgcaatacagtggatacagtttgctgtggctactctgagtgttataggctccagttcactcctcacctacatcgcgttccagagcccacagaagtctgcagagataaagccactcttgtatctgagcttctctgacctgctcctgggaatttgctggctcgtaaaggcacttctctatggaactgcagcagcccacaaggacagcatctgctataacctacagaccgtgggggagattttctaccttgcctcactcctctacaccgtcagctacatctggtgtctgtactcggagctgaggaggaagagcggccagagtgagaggagcacggttgcccaggtgacagattatgtttgccaaggtggtcacatattgttccttttgtcaagcctgatacctccgctgctgatgacgcctgtgttccttttgggcaatgtcaatgaatgtttccacaacttcagtgagagccacaggtgtatcctaatgcactcaccgccatcagccacgcctgagcccctgccttccaccaatacatctgtctgcagcacactctatttttatggtgtcaccattttcctggccagctttttcttcagcttcctcaccatcatggtcttactcgtccatgcccagatgttatataagaagttttgtraagtcgacgggccttctggagagtgagcagtgggcagtgattcacactgtgggccagcgagtgcgtttcttnncagtggcctttgtgtgctgctggggtncagctgtctccctgctgatcatgaagctgacagagcctcaggagaccttcctgcacatggccctctctgtgctccaggctctctcagcagcatcccaggggctgctcaactgtggcwtttatggctggacncagtgcaagttccagcagataaagcgtgagcctcaccgtgacgcagacacgcagaccccwctgctgtgctctcagaagagaatttatagtagaagcccaaacctactggagtctcctcttgcccttgcttccagctcctccaccgttctttgagaccctagaatgggaatgtcagggagtgaatgtttcacatgagtggactctcctctggccaaacccgagactggagagctgaaaggaaatgtgggctcagggtcagtagcaattctgggtgaattcagaagcagctctctaactgctccttcagagatatacagccgctttccatmtaagagactcgttactttgacaagaaaaaataaatctcaggttcataaaaaaaaaaaaaaaaaaa

SEQ ID NO: 280 - DNA, 764 bp, Mus musculus
aaaagtgggccccgaggtcgcgtcttgcccaagtctccgcggtccccagcgctcgctcgcgcggtcctgccacggccttcctgctgcccgcgccatgggcctcagctccaactccgctgtgcragttgagtggatcgcggccgtcacctttgctgctggcacagccgctctcggttacctggcttacaagaagttctacgctaaagagaatcgcwccaaagctatggtgaatcttcagatccagaaagwcaacccgaaggtggtgcatgccttcgacatggaggatctgggggataaggccgtgtactgccgatgctggaggtctaaaaagttccccttctgcgatggggctcacataaagcacaacgaagagactggcgacaacgtaggacctctgatcatcaagaaaaaggaaacctaatggacagttgcgaggctgcacccagcgtgttgtgatgtcacctgctgatttacgtagaatggcacccaacccacmbgtctgattggcctccccggttctagatgtggttggtccctgcaaatcacagctctcatatccatggcatcggccttgctactgaaacatgtggtgcacgtttgttgaaagaagaagaaaggctaaaccaacctcgtgctatatgggttattttggtcttgtaaggatccgttcctttaaaataatggtcttagaatatagttgtatcttgaggttaaagtattaaattattccaaaatcatgtacatgtaaaaaaaaaaaaaaaaaa

SEQ ID NO: 281 - DNA, 1127 bp, Mus musculus
aaaggagcaacgcgggtcttcccgctgttgcttgtcgcggccacggccgagcatacgtccccggcctgaggtggtggtggcgagcccaccgccgtttgctgcgacctcatggaaggtggcggaggaagtagcaacaaatccaccagcgggttagctggcttcttcggagccggaggagcgggttactcgaacgctgatttggccggcgtcccgctgactggtatgaaccccctgtctccttatttaaatgtggatccacgctatctcgttcaggatactgatgaatttattttgccaactggagctaataaaacccgaggcagatttgaactagctttctttaccattggaggatgttgcatgacaggggccgcattcggggcaatgaacggtcttcgtttaggattgaaggaaacccagagcatggcctggtccaaaccaagaaatgtacagattttgaatatggtgactaggcaaggagcactttgggctaatactctaggctccctggctttgctctatagtgcttttggtgttatcattgagaaaacacggggtgcagaagatgacctcaacacagtagcagctggaaccatgaccgggatgytgtataaatgtacaggtggtcttcgaggaatagcacgcggtggcctggcaggactgacactcaccagtctctatgcactgtataacaactgggagcacatgaaaggttccctgctccaacagtcactctgaagatttttggcacctcaggaatgggggacacttcatagtcatccagatcaattaataagccagtctggagttgctttctgtcttgtacctacagttactttgaacaattggagactctgatttgctgtgacaaagatcatggatggttagcctggactgcactgcttgccctcagtgagtggaagccaaccccttggtggctcactgagtatgtgttctcttctcctttggagatgatcttgcaccccaaactggaaaaccacttactctccaaattcaggccatactttttattaccatgttaattcatttaaaaaatctaaaacttaaatgttgccctgttttccaaataaagggtgaaaacagaaaaaaaaaaaaaaaaaaaaaaa

SEQ ID NO: 282 - DNA, 1292 bp, Mus musculus
  misc_feature (237)..(1132): n = any nucleotide
agggcctccgcacttccaagtcatctgccggctacttggctaccagggcattgcagtagtcatggaagagctgctgaaggttgtcaagagcctgctgcaaggcacaatcctacagtacgtgaaaactctgatggaagtgatgcccaagatctgccggcttccgcgacatgagtacggctcwcctggcatcctggagwtcttccaccaccagctgaaggacattgwggagwatgcasnagctgaaaaccgtatgcttccagaacctgcggsaggtgggmaatgctgyyctcttctgcctgcttatwgagcaaagcctgtctttagaagaagtctgtgacctgttgcatgcagctcctttccagaatatcttacctcgaatccatgtaaaagagggggagagagttgatgccaaaatgaaaagactagaatccaagtatgccccactgcaccttgtcccactgattgaaaggctggggaccccacagcaaattgcaattgcaagagagggggacttgctgaccaaggagcgtctctgttgtggtctgtccatgtttgaagtcatcctgacacggatccggacctttctggatgatcccatctggcgtgggcccctacccagcaatggtgtcatgcacgtggatgagtgtgtggagtttcacagactatggagtgccatgcagtttgtttactgcattcctgtagggacacacgagtttacagtgggagcagtgttttggagatgggcntccwctgggctggctgcatgatcattgtacttcttggacagcagcggcgctttgctgtgttggatttctgctatcatcntctcaaagttcagaancatgatggcaaagatgagatcatcaaaaatgtgccattgaagaagatggtggagaggatccgcaagttccagattctcaacgatgaaatcatcactatcctggacaagtacttgaaatctggtgatggcgagagcncncctgtggagcatgtacgctgcttccagccncccntcctccagtccctagccngtagctgagggctaagctcncagcatcccagtaactgatggcatgtttgtctttatgtaaactatattgaaatttttaggggctattttccnttatgtctgaacctacttttgatctgaaagcttaactttataaaatttacttatttttctatactaaaattgtatatgcctttggaattgggaagtctgagaatacggttactagttgttgccnttacggtattaacaagtattttcatgccgaatattacagcagnta

SEQ ID NO: 283 - DNA, 699 bp, Mus musculus
  misc_feature (29)..(29): n is a, c, g, or t
aaagcctgtgtacgccaccatcggctyynggtatcgtcaacacggccttcactgtggwgtcgctgtttgttgtagagcgagctggacgacggaccctgcacctcattggcctggctggcatggcaggctgwgctgtgctcatgaccatcgccctggccttgctggaacggctgccttggatgtcctatctgagcatcgtggccatctttggctwwgtggccttctttgaagtaggccctggtcctattccatggytcattgtggccgagctgttcagccaggggccccgtcctgctgctattgctgaggctggcttctccaactggacctcaaacttcattgtgggcatgtgcttccagtatgtggagcaactgtgcggcccctacgtccttcatcatcttcacggwgctcctcgtgctcttcttcatcttcacctacttcaaagtccctgagaccaaaggcctgaaccttcgatgagatcgcttccggcttccggcaggggggtgccagccaaagtgacaagacacccgaggagctcttccaccctctgggggcggactcccaagtgtgaggagccccacacccagccccggcctgctccctgcagcccaaggatctctctggagcacaggcagctagatgagacctcttncgaaccgacagatctcgggcaagccgggcctgggcgcctttcctcag

SEQ ID NO: 284 - DNA, 797 bp, Mus musculus
  misc_feature (19)..(790): n = any nucleotide
aaagcttacccagcagsrngagtagatttgtatcaacaagagaaggcacggatgggctcagcgggactcagagttcttgccttggcgtctggtccggaactggggcagctgaccttccttggcctggwcggaatcattgaccctcctcgaactggtgtgaaggaagctgtcacaacactcattgcctcaggagtctccatyaaaatgatcactggagattctcaggagactgcaattgccatcgctagwcgtctgggattgtactctaagacttcacagtccgwgtctggggaagaagtcgatacaatggaggtgcagcacctttcacagatagwgccaaaggttgyagtattttacagagcaagcccaagacacaagatgaaaattattaagtctctacagaagaacggggcagttgtagccatgacaggagatggggtaaatgatgcagttgctctcaaggctgcagacattggagttgcgatgggccagactggcaccgatgtttgcaaagaggctgcagacatgatcttggtgaatgatgatttccaaaccatcatgtctgcaatagaagagggtaaaggcatttataataacattaaaaattttgctagatttcaactgagcacgagtatagcagcattaactttaatctcattggctacgttaatgaactttcctaaccctctcaatgcaatgcagattttgtggatcaatattataatggatggacctccagctcagagccttggagtagagccagnggataaagatgtcattcgaaaaccgnctcgaaa

SEQ ID NO: 285 - DNA, 569 bp, Mus musculus
  misc_feature (429)..(565): n = any nucleotide
ttttttttttttttttttttttttttttttttttttttttgccyttaaatgattgagtttatttgtgkgttagtaagaaagcccyayaaccataaatagtacaatatttaaaagtaaaaaaaatatttatatccatctaagacagacagtgtatttgtacattagaattctttaagtgcagaaggtggttcaggttttgccttttgkattttttaattcagtacttgcccatatgttgaagttcactcaattgcaaaagccaattcaaatcaaggaatgtgttattcaaagktgcataatttcatttgggactaccaacaaagttcaaatcaagtctttaacattaatgggtcatttttagtcaatggaaagttaaaaagtccttaagtcttcattatcttcttgaacttgcccttctaaatgattctnagactgcantncctagtttgcnaattaaaaaaaaatgcnntatgcatcatacttnaaggaaagacgatgaangctnaaccagcnttcttcctggaccttctcccggctcctttcanccttctttataaccctcagncaca

SEQ ID NO: 286 - DNA, 1171 bp, Mus musculus
aggattggcggggttctctttgaccacataaatcaattctttcttttgggagcctcaatggtggcaactgcagcgggactttatctcatcccgttctgcaagacagcagtcttattgatcatcaccatgtctgtctttggagcttccgttggtgttgtggatacaggtgcaaatgttctcatcttggatctctggggggacaaaggagccccacaaatgcaggccttgcacttcagtttcgccttgggtgcctttctggctcccctgctggctaagttggcctggggtacagcacctgctcagaaccacaccgagtccgaccttgacactctgatgctgaaccgatcctccaacggcacctcagactccgtgtttgcggtacccgatgacatgaatctgctgtgggcatatgcttccattggcacctttattttagtagtttctgtctttctgtttggtctgttttgtaagaaacattcaaggcagaaaaaacccagagcatctgctgagggagctcgaagggctaaatatcacagggccctgctatgcctgctcttcctcttcttcttcttctatgtgggagccgagataacatacggctcttacatattctccttcgccaccacccatgttggcatggaagagagcgaggcagctggcttgaattccatcttctggggaacctttgcagcctgcaggggcctggccatcttctttgcgacattcttacagcctggaaccatgatcgtgctgagcaacattggcagcctggtctcatgtttctttctggtactttttgacaagagccctctttgtctctggatcgcgacttctgtgtatggagcctcaatggcagccacgtttcccagcggcatctcctggattgagcagtacaccaccttaactgggaaatctgcagcattctttgtaattggctctgccctgggagatatggccattccagcggtgatcggaattcttcagggacactacccagatctgccagtagttctgtacacatgtctgggctcagccatattcacagctattttatttcctgtgatgtataaattagctaccttgcccctgaagagagaggaccagaaagctttgcccactagttctagactgtgaggaagagactacatgagaacttaaaaaaaaaaaaaaa

SEQ ID NO: 287 - DNA, 768 bp, Mus musculus
atgcaatggcggacgtgtctgagaggacgctgcaggtgtccgtgctagtggctttcgcctctggagtggtcctgggctggcaagcgratcggcygyggaggcgttacctagactggaggaagcggaggctgcaggwcrrgctggcaacgactcagaaaaagctggacctggcctgagcacgcgctgcagcccgagtccgccgggttctcactccctaagcccaacgcagcccggatcgtgggagccgcgcgacccaggagtcgtccttgcacggcttgcaagaacatggcttgcttcagaaagaaaatagttttgtcttctctaacaacttactttcagcttgtcgaagatgaaaataaaaagcactggagagaaataatttcttgcactttatgaatctatttttaaaataaaaaaattaaacatctttgaatctttttcctcctcacaaaagaaagcagtatttttgcctaccattcagtttgctgcagtaagagatttggagcctgaaagcagagactttctgatggaatctcaccttggtacagcctggaggcagatctgatcaacggaccattatgagtcatttttctagacatattcagaaaacctaggagctgtgtcaaatgcctgaattaagcattacaaatgcaagatatttgcactttgaagaatgtagagagtaaaaaaactaaaattaaaaaaaataatgcatgtgatataacggaatatatatgtgaaagagaaaaaaaaaaaaaaaaaaaaaaa

SEQ ID NO: 288 - DNA, 836 bp, Mus musculus
agatagttttgaacttccgattcccctgcctccccaccccagggctgtgattgcaggtgtgcgcttgggactgaccccaggctttgtcggtgctagggcagcactctgcgactgaattaggtcccagccacttctctgtcttttaaaagaacaaaacattgctaaatgtgccattgttgctttgagttttaattcttttttttttctttctttcataaaacattacagtcttaagatattaaagacttttattctggttctatttctgtcttttcactcaaaactggttttacaaatgatgccttgtttacagaaagctctctaccacagggcctagtcatgtgtaaagtctcagtttctctctggagtatcttggagcctagcacactggctttaaaggacacagctaagaagctgatatcttgacagtgtttgtagacctttgttataaaaatgaatgtcctggaaagggttgggagggagttcaacaacaaagaaacaagaatgtcatgtttaaatttaatagttgtctaaaatgtcatctcaagtcaagtcactggtctgtttgcatttgataggtttttatactaactagcattataagattatttcataattagaaaatacctgtggatatttgtataaaagtgtgaaataaattttttacaaaagtgctcatcgcttgttaacacagcatcatgtatgtgaaagcaaactctaagattataaatgacaacctgagttgcctttctttgtatttcatcaagccaaagtaaagctttcaatatttaaaaaaaaaaaaaaaaaaaaaaaaaaaaaaaaaaaaaaaaaaaaaa

SEQ ID NO: 289 - DNA, 787 bp, Mus musculus
  misc_feature (712)..(733): n = any nucleotide
agcatccctccatggcctctgcattggctyctgctttctgacctgcttgagttccagtcctgacttccttggtgatgaacagcagtatggaagtgtaagccgaataaaccctttcctctccaacttgtttcttggtcatgatgtttgtgcaggaatagaaaccctgactaagacaaattggtaccagcagagtggggtattcctgtgacaacctgaccatgttttggggaggactgtggatggactttggaactttgggcttaaagatccatccgttgttaagagctctgtcagatgttgtgtaggagcttggaagataatgttgagaacactgcagaagatggaggtctggtttgtgaaatttcagagggaaaattaaagactcttttcagggccattgctgttttgaatgtgaagattctgtagttctggwtagctggggctgaagaatcagctgtgattaacaagataccagaactaccaaagcaaaaactttgcattactgggactattgatgctggttagctggagctaagaaattagcggtgattaagaagagaccagcatcattgaggtgacatcttctgggaagtgttttctgaaagcacaaagatgctgtgttccagagatggccaaggttgtactcctgctgcagcaggacttggtaatatgtaagggtcacccaggtggtactggttttgaaggcatgaangggtcacgcaaagcagctgangctcggcactgtgagaggccatggaaggccattgctgaaagtacagcctcagtt

SEQ ID NO: 290 - DNA, 576 bp, Mus musculus
aaacggcaacacttcggtcaggatatcttctaccagacaccaaggcgtatggagatagaatagaaagaatgcttcgcctcagtttaaacattgaccctgaagcacaggtggaggaagaaccagaagaagagcctgaagacacctcagaagacgcagaagactcagagcaggatgagggagaagagatggatgcagggacagaagaagaagaggaggaaacagaaaaggaatctacagagaaggatgaattgtaaattatactctcgctatgaatcccgtgtggagagggaatgtgaagttttgaagtcatttcttttgagagacttgttttggatgcttccccaagccyccttctcccctgcmctgtaaaatgttgggattatgggtcacaggaaraagtggtttttttagttgaattttttttaacattcctcctgaatgtaaatttgtactatttaactgactattggtgtaaaatcttgtcatgtgtataaaaataaaaaaagrtccccaatwaaaaaaaaaaaaaaaaaaaaaaaaaaaaaaaaaaaaaaaaaaaaaaaaaaaaaaaaaaaa

SEQ ID NO: 291 - DNA, 589 bp, Mus musculus
aggggagatcctgagaacagaccagtggcctcggaggctcttcatgcagctcattccgcagcagttgctgaccaccctcgtgccactgttccggaattcacgcctggtacagttccacttcactaaggacatggagactctgaagagcctttgccggatcatggacaatggcttcgcgggctgcgtgcacttctcctacaaggcatcgtgtgaggtgcgtgtgctcatgctcctgtactcctcggagaagaagatcttcatcggcctcatcccgcacgaccagagcaactttgtcaacggcatccgtcgcgtcatcgccaaccagcagcaggtcctgcagcgaagcctggagcaggagcagcagcagcgagggatgggtggctagaggatgcctgggctgggcgggccacagtcccagacgaggccccagtggagactggtcagctgcttctgagcaggggctcctggcgactgtacctgcccagcaacctggaggacagcgtcctgaggtctcccaaggatggtccttgcccctgtatgtttccctaataaagccttttaccccaaaaaaaaaaaaaaaaaaaaaaaa

SEQ ID NO: 292 - DNA, 755 bp, Mus musculus
ggctgcttcgtggagcagagaggtgcatcaccaggttcccgatgaacccagagaaccctccaccgtatccgggccccgggccaacagccccatacccaccttatccacaacagccaatggggccaatggggcctatgggagccccaccttcctcaggggtacccctacccaccacctcaggggtacccctatcaaggatacccacagtacggctggcagggtggacctcaggagcctcctaagaccacagtgtatgtggtggaagaccaaacgaagagacgacctgsgcccatccacctgcctcacagcctgctggactgctctgtgttgctgctgcctctgggacatgctcacctgatcacctgatgagcccagctcttccgcttggccgctctgtgccacctccgataagtgtgcctggccccatctcttctgattgctataaagtggctagctctgcgcagacacctctactttctgtcctatggagttttagattagtgagggttactgctatttagttcagtgacttgatctttttaatgttcaaaactcatttcttactgatctttaaaaaatgtgctaaataatttacttttttggccaaaggcttagttatgaaatatatatttataatgtttaaattatacactcaaggtaatggcaatatgtgacacactactagatctctctgctcataccctttatgtttcaataaatctgtccaaaatctggaaaaaaaaaaaaaaaaaaaa

SEQ ID NO: 293 - DNA, 724 bp, Mus musculus
ctggaaaggcgcggagcctgctgctgccatggwggctggtggctgggtacactatgtctagactgggggccctgggcggctcccgcgccgggttgggactgttacttggtactgccgccggccttggattcctgtgcgtcctttacagccagcgatggaaacggacccagcgccatggccggagtcacagtctgcccaactccctggactatgcgcaggcttcagagcgtggacgccaggtgacacagtttcgggctatcccaggtgaagctggagatgctgccatactgcccagcctctcacaggaagggyaggagaaggtgctggaccgcctggactttgygctgaccagtcttatggcgctgcggcgcgaggtggaggagcttcagagaagcctgcaaggactagctggggagattgtcggkgaggwccgctctcatatakaagagaaccagagagwggcccggcggcgcasgttcccttttgccagagagaggagtgactccacgggctccagctctgtctacttcaccgcctcctcaggggccgcactcacagacgccgagagcgagggaggctatacaacagccaacgcggagtctgattacgagcgggactccgacaaggagagtggagatgctgaggacgaagtgagctgcgagaccgtgaggatggggaggaaggactctctggacctggatgtggaggcggcgtccagtccgtc

SEQ ID NO: 294 - DNA, 1263 bp, Mus musculus
agcgctgggtctgagtgaccaaaggcagtagcgctcgcggagatcacccgctggccctcgatcaccatgtcggccttcgacactaaccccttcgcggacccagtggacgtaaaccccttccaggatccctctgtgacccagctgaccaatgctcctcagagtggcctggctgagttcaatcccttctcagagacaaatgcagcgacaacagttcctgccacacaagctcctgggccctcccagccagcagttctccagccctcagtggaaccagcacagccaacgccccaggctgttgcagctgcggcccaggcaggcttgcttcgacagcaggaagaactagacaggaaagctgccgagctggaacgcaaggagcgagagttgcagaacactgcagcgaatttgcatgtgcgagacaacaactggccgccactcccctcatggtgccctgtgaaaccctgcttctatcaggacttctccacggagatccctgctgactaccagcggatttgcaagatgctttactatctctggatgttgcattcagygactctgtttctaaacctgcttgcgtgcctggcctggttcacaagtgacgcagccaatggaacagcctttggcctctccatcctttggtttctgatcttcactccctgtgccttcctttgttggtaccgacccatctacaaggctttcaggtctgacaactctttcagcttcttcgtgttcttttttgtatttttttgtcaaatagggatctacttcatccagttgattggcttgcctaacttggggaccagtggttggcttgcagccctatctacgatgaaaaatgggcccttagctgtgaccatcatcatgatggtagtggctggcttctttaccctctgtgctgggctttcactcttcctcctgcagcgggtacacgccttctaccgccgaacaggagccagcttccagcaggcccaggaagagttctcccagggtatcttcagcagcagaacctttcgcggggctgcctcatctgctgcccgaggagcctttcaggggaattaggcttcccgactctccttccctgagccctggcctctctgtcaactgtcttctgagctgcactttcctcgggtgccttatgcagtgactacaccagcacagacctggcagggatttgtcttgtccctgctcctccctcagtagtcagctttctcctctacaagagggaaggagaggaagggacaaggactttaaaaaaaaaaaaaaaaa

SEQ ID NO: 295 - DNA, 1229 bp, Mus musculus
  misc_feature (890)..(890): n = any nucleotide
ttcgtcactgccaggctgaactggagaggctttcagagtccctgaggaagagcatctcctgtggagctttctctgttcctgggggtcacagcctctacttagaagccaggaagaagattgagctgggctaccagcaagtgccgaggaagggagtgaaggcaaaagaggttctcaagagtttcctacagtcacaggctattatggaggactctatcttgcagtcagacaaagctcttacagatggagagagagccatagcagctgagcggacaaagaaggaagtggctgagaaggaactagagctgctgaggcagagacagaaggagcaagagcaagtgatggaggctcaggagagaagcttccgagaaaacattgctaaacttcaggagaagatggagagcgaaaaggagatgctgctgagggagcaggagaagatgctggagcacaagctgaaggtccaagaagaactgcttattgaaggattcagagagaaatctgatatgttaaagaatgaaataagtcacctgagagaagagatggaaagaacaagaaggaaaccctcactgtttggtcaaatccttgacacccgttggcaatgcgttcattatgattttaccaggagctggtaaactatttggtgtggggctgaaattcctcggctcactaagtagttagtctaaagagtttctgggaaggaatttataaaatccaggcttctggtattttgataccaaactttttgctgkgatttttaagtacacattctagtttaataaccacggttacmaagaaagtaagaacttycattaatagctaaattgtcagatactgcttcctcggagtctaattctgtgtcagttcattacctattatatttaaggttaaagatncaaaccaatgattgaagaatataagggtctggcgtccataaatcctgtcaaatttcagtgatgttatctaaataataaagagggaggccagtcagaatatagtgaaattatttctagaaaagatgtaagtaaatcaaacagaaacatggccaaatgaagacmcattggcatatgtatcccagttgtacactacagataaattgttaaaaagagctcttgttaaaacgcmcaagtgggttttcataagagaatgtatgaggaaaatgtttgattgataataaaaggcatgcagattaaaataaaaaaaaaaaaaaaaaaaaaaaaaaaaaaaaaaaaaaa

SEQ ID NO: 296 - DNA, 1318 bp, Mus musculus
agaacgcctctggggagcatggcatcgtggttttctctttgggatccatggtctcagagattccggagaagaaagccatggaaattgctgaggctttgggcagaattcctcagacggtcctgtggcgctacaccggaactagaccatcgaatcttgcaaagaacacaattcttgtcaaatggctaccccaaaatgatctgcttggtcatccaaagactcgggcattcatcacacactctggctcccatggtatttatgaaggaatatgcaatggagttccgatggtgatgatgcccctatttggcgatcagatggacaatgccaagcgcatggaaactcggggagctggggtgaccctgaatgtccttgaaatgactgctgatgatttggaaaatgcccttaaaactgtcatcaacaacaagagctacaaggagaacatcatgcgcctctccagccttcacaaggaccgtcctatagagcctctggacctggctgtgttctgggtggaatacgtgatgaggcacaagggggcaccacacctgcgcccggccgcccatgacctcacctggtatcagtaccactccttggatgtgattggcttcctcctggccattgtgttgacagtggtcttcattgtctttaaatgttgtgcctatggctgccggaaatgctttgggggaaaggggcgagtgaagaaatcacacaaatccaagacccattgagaagtggggggaagtgaaggagaagtattagttcattatctgatcagttgaacyttggaaacaagtgttagatccatattgtttttgttagggaaataattcaccatacattatacattcagcacatttaaaaataataataataacaatctaattgctggccacacccatcagggaaggttctagtatatgtgatgtgcttttccagtatcttcagtctagacaactcttgccatctgttggtaatttacagaaagtctggcactctgctttcagtgacagccccacagtttcccctgctcccgccagctgatggttttctcccctggattctcagactgccgtggccttctccagtgttagtcattcttcattgtgttcatgcattatgggtagcaagacctttggagctttgggagaagagatgaggctgtgacactgatggccctgtgtttgagataataattgttgcttgtgccggaatttgatgaaaaccaaagtatgttctaaggcaagtacatcttctattgtgttcccaaaccaagaacttatcaataaattcatataaattgtatcactaaaaaaaaaaaaaaaaa

SEQ ID NO: 297 - DNA, 817 bp, Mus musculus
  misc_feature (703)..(802): n = any nucleotide
agagaagcagacatggctcaatttgcacaggtcatggctgaagtgggcgactttggtcgctttcaggtgcggttgaccatcctgatgggcattcccaatttcctggctgcattcttcatatttggccaagtcttcatggtccttgatgaggctcaccactgttcagtgtcctgggttaagaaccacactttcaacctaagtgccgctgagcagctggctataagcatccccaacgacacagcgggcagacccgagtcctgcctcatgttccggccacctcctgacagtgccagcctggaagacatcctgagccaccgcttcaatgagacacaggcctgtgactcaggctgggactatcctgagaaccggcctcagtccctaaagaaagagtttgacctggtgtgtgatcgaaagaacctgaagaagacctcccagtcggtgttcatggctggtctccttgttggggccctggtctttgggcctgtctgtgactggattggccgcagaccctccctcctgatgcaggtgcttctgtcaggcatcacaagcatggccacagccttcgtgtccagctttgagctctacttggccctacgctttgtcttggctactgccaatgctggatttttactaagtaccaatgtcctgatttcagagtgggtggggccatcttggagaacacaagccgtggtctttggcccanagcaacgttgcccttgggcagatggtgttagcaggactggcctatggtgtccgaaacttggagacttcttcagatacanggacccgacccgcttactgntcttcttctatttct

SEQ ID NO: 298 - DNA, 613 bp, Mus musculus
  misc_feature (514)..(514): n = any nucleotide
agcgggcctgagccgagcagctgcgcgacgtcatggacaactccgggaagcaggctgaggctatggcgctgctggctgaagcggagcgcaaggtgaagaactcgcagtccttcttctccggcctctttggaggctcatccaaaatagaggaagcatgcgagatctatgccagagcggcgaacatgttcaagatggccaagaactggagcgctgctgggaacgctttctgccaggctgcccaactacacctacagctccaaagcaagcacgatgcagccacctgctttgtggacgctggcaatgctttcaagaaagctgacccccaagaggccattaactgtctgatgagagcaattgagatctatacagacatgggcagattcacaatcgcagccaagcaccacatctccatcgctgagatctatgagacagaactggtggatgtagagaaggccatcgcccactatgagcaatcctgcagactactacaaaagggagaagagtccaacagctnagccaacaagygtctgctgaaggtggctggctacgcccgaacagctggagcagtaccagaaggctatcgacatctattgagcagggggggaccagtgcc

SEQ ID NO: 299 - DNA, 930 bp, Mus musculus
  misc_feature (421)..(908): n = any nucleotide
aaagttgttcttctcgtggttcccagtggcgagaggaggaggaagcccggagcggagcggggcgkctggggggggtggacccgccgcggctgctgctgccaccgccgccgccgccaccaccgctcgtggggctcgtggcgtgaggaaggaggacgagtgagaccccggggcgagcgggcggcggcgccgctgctgctgctgctgctgcgggagggtcggcggcgggacggcgatggcggatatcgacaaactcaacatcgacagcatcatccaacggctgctggaagtgagagggtccaagccaggcaagaatgtccagctccaggagaacgagatccgaggactctgcctgaagtctcgggagatcttcctcagtcagcctatccttttagaacttgaagcaccactcaagatatgtggngacatccacgggcagtactatgatttgctccgtctgtttgaatacggtggctttcctccagagagcaactatttgtttctcggggactatgtggacaggggcaagcagtccctggagacaatctgcctcttgctggcctacaaaatcaagtatccggagaacttctttcttctcagagggaaccacgagtgcgccagcatcaataggatctacsgattttatgatgagtgtaaaagaagatacamcattaagcttgtggaaaacgttcacagactgktttaactgcttgccgatagcaagccatcgtggacnaagaagatawtctgctgtcatggagggttatcaccagawcttcaatctatggagcagaattcsgcsgaaatatkagaccaaytgatgtaccagawcaagggccttctttkggaaccnttgggggcctganccccataaagaggcnttaagcttgggtggaaaatgcccgaggagnggccttccaattggtgccaaat

SEQ ID NO: 300 - DNA, 668 bp, Mus musculus
aggatcatccgtctcgcctccagttcttcaccggctgagctgggtacggacatctcagtgaccacgagctaacatagcacgtgaaactgacctcaggaagaacgaagcctggagacccggagtgcgacagcgacctccggactgaagtggaagaagcgtagcgaggccttcgtgtcctcgctgtggggggcggggcttgttgtgcaggcggggctcacgtggtactttggtactttggatgactgaagggaagcttggtacccgcctggtcactacccggaagtccagccggagtagcactaccctggagtccacccggctgtgaagcggtggtgctcttaggggtgcttgacgccctcattgttagaaactgatgggtactgttggccgattcctagacccgcttttgacctacgtgataaatgtgtttccggaactctgccaaaaagtgttctggaggtttttgtttgtttcggagccaaggcctccctcgtagctcttaatggcctgaaacactctttgtagcctagactggcattgaagtcaagttagccctgcctttgcctccctgggctctgggattaccctgcctttcttgattatttctatttgaacaaatatgaacaaactatgtggccaccacaaaaaaaaaaaaaaaaaaaaaaaaa

SEQ ID NO: 301 - DNA, 876 bp, Mus musculus
agatggagcctgcgtttcacagaggggatctccttttcctcacgaaccgagttgaagatcctatacgcgtgggggagatcgttgttttcaggatagaaggaagagagattcctatagtgcaccgagtcctgaagatccatgaaaagcaagatgggcatatcaagtttttaaccaaaggagataataatgctgttgatgaccgaggtctctataaacaaggacaacactggctggagaagaaagatgttgtggggagagcaagagggtttgttccttacattggaattgtgacgatcctcatgaatgactatcctaaatttaagtatgcagtactgtttctgctcggtttatttgtgctggtccatcgtgagtaagaagtcggactccctgttcctaggaagctgctgtgcttgttgttactgaatgttggagtagatcctgatctgtgattgcggattttcggaggacacacacgttggcacttcttggtagccctggtttgcattgctttgtgtttccacaccagaggctgtgtgggcgggtgcatgtgcaccgtggagtgcacacaaggggactgtcaatcacagggtttcatatgttgtcattgtcactctttcacatttttgtacatcagtgaattttttatattaaaaggttgagccaaagcccccagtgtttgtattttgaagccaagcttcactttaaaagtgcctacagagttctgtaaatgaaaacacagctctgcatgagttcaaacctgtcgktcctycttacagtaggaatggcacatattgaggcggkcataagtcttaacttttcaaaattttaaataaaagactttgcacatttaaaaaaaaaaaaaaaaaaaaaaaaaaa

SEQ ID NO: 302 - DNA, 721 bp, Mus musculus
  misc_feature (361)..(361): n = any nucleotide
aaagttcttctctcacgtgggttggctgcttgtgcgcaaacacccggctgtcaaagagaagggcggaaaactggacatgtctgacctgaaagccgagaagctggtgatgttccagaggaggtactacaagcccggcctcctgctgatgtgcttcatcctgcccacgctggtgccctggkactgctggggcgagacttttgtaaacagcctgttcgttagcaccttcttgcgatacactctggtgctcaaacgccacctggctggtgaacagtgccgcgcatctctatggatatcgcccctacgacaagaacattcaatcccgggagaaatatcctggkttccctgggtgccgtgggcgagnggcttccacaactaccaccacaccttycccttcgactactctgccagtgagtaccgctggcacatcaacttcaccacgttcttcatcgactgcatggctgccctgggcctggcttacgaccggaagaaagtttctaargctactgtcttagccaggattaagagaactggagacgggagtcacaagagtagctgagctttgggcttytgagwtcctgtttyaaacgktttctggcagagatttaatattctgttgattaactaacaactggatattgctatcggggtgttaatgatgcatttaacctattccggkacagtattcttataaaatgagaaagctttgatcacgttttgaggt

SEQ ID NO: 303 - DNA, 616 bp, Mus musculus
tcatacaaacaccccagtacctccagcagctccaggagtcaaaaacaccagcagtggaccgaggtcctacagctgcctgcagccatgttggccttcagtctgctcgtgctgggtctgctcgcggaagtggcacctgccagctgtcaacaaggtctaggaaaccttcagccctggatgcaaggcctcattgctgtcgcygtgttcttggtcctygttgcaatcgycttygcmgtcaaccacttctggtgccaggaggagccggagcctgsgagcacagtsmtgaycrtyggaaacaaggcagatggrgtcctggtgggaayrgatggmagrtactcytcratggcrkcyrgtttyaggtccagygagcayragaatgcctaygagaatgtkcysgaggargarggcarggtccgcagcacmccsatgtgrcragcykctmysyrkmcswmgtccccagrctacgcwtagagttgatccccaccatctcacccagccactgctctacaggaatctactgaaacaagctgactycctcacctctctagaatcacagtcatctyaggccaggactcagaccaagcccagcacaccaatgtgctgtgacccykggctttcc

SEQ ID NO: 304 - DNA, 1225 bp, Mus musculus
  misc_feature (755)..(861): n = any nucleotide
aaagcgctcctcgagggtcggtcccgccacgtctcttcccggtctctctggctctggagtgctttccccccgcggagggtgggagcgcggggacagacgggcgagatgagcaccatgtttgcagacacgctgctcatcgtctttatctccgtgtgcaccgcgctgctcgccgagggcataacctgggtcctggtttacaggacagacaaatacaagagattgaaggcagaagtggaaaaacaaagtaaaaaattagagaagaagaaggaaactataacagagtcagctggtcgacaacagaagaagaagatagagagacaagaagagaaattaaagaacaacaatagagacctgtcaatggtacggatgaaatccatgttcgcgattggcttttgttttactgccttaatgggaatgtttaattccatatttgatggtagagtggtggcaaagctccccttcactcctctgtcttacatccaaggactatctcatcggaacctgctgggagatgacaccacggactgctccttcatcttcctgtatatcctctgtaccatgtcaattcgacaaaacatccagaagattcttggcctcgccccttcacgagctgccaccaagcaggctggtggatttcttggcccaccacctycgtctgggaagttttcctgaaggaaagcagaattctgaatttcctgtcatactttttagacattcacatcagacttacmgagcacctggccacawtctaggtnkgkgtwatttctcagctatggatatgawcmaatgagaaccctgmtttaactaawgggawartgctatggtcaccggatggcttctttnagtaataagtgsmcycnntctggktaccatttggawggcttaatgtaaccccmaaccatcaatctttcttgtctyacttgagtatttcagtgtatgtgccatatacacagaagcagactgtccatgaacattaggagaaaaatagtttatgtaacaacttcactgtaattagttgttgtgttttttaaaaagccaaatatgtcactttgtgttcgacaatgagccaaatttttagatgtttcagttagagaaatgtttgatttttttgcatattaaaaagagtattttctgtaatctgaacataaggataagcattggcccaaaaagctaaaataaagttttgtcatgttcttttaccaaaaaaaaaaaaaaaaaaaaaaaa

SEQ ID NO: 305 - DNA, 677 bp, Mus musculus
  misc_feature (198)..(198): n = any nucleotide
aggcgacatggggaggcgcggaggcgacaccggaagtggctgtggtccgggtcggcccgagggggactcagcgcccgcagcaaccacccgcgcggcggcggtcagagctcatacccgtgcgggtgggcgtggcgggcgccgggacacagctccgactgttcgctgtcggcgcggrgmgkcgccgcgtcgctcgccttnaccagctgccatgagcgagcgcctccgccccagaaaaaggagaagaaatggcagtgatgatgacaaccaccctcctccccagaccaaaaggagcagtaggaaccccatcttccaggactcctgggacacagagtcttcaagcagcgacagtggtgggagcagcagcagcagcagcatcaacagcccagacagggccagtgggccagagagcagcctgagccacaccatccccggatcctgccccagcaccccccagccgatgcctgagcagtctgcactatgccaaggcccttacttccacatcaaccagaccctgaaggaggctcactttcatagcctacagcaccgaggccggccgccgacatgatgctcctccggcagtttcttgccttctgtgaagggacagcactgtgcagattggatatttcaacttaatgatttgtttttaaaagttgcacacagaaaaaaaaaaaaaaaaa

SEQ ID NO: 306 - DNA, 1002 bp, Mus musculus
tgctgaggtgggagtgggcccagcgcccggtggccgcagctcacgcgcaacctgcgccatggccgcctccgccgcctcctccgagcatttcgagaagctgcacgagatcttccgcggcctccttgaagacttacaaggggtgccggagcggctgctggggaccgcggggacagaagagaagaagaagctggtcagagattttgatgaaaagcaacaggaagcaaatgaaacgttggcagagatggaggaagaactacgatatgcacccctgactttccgtaaccccatgatgtctaagctgcgaaactaccggaaggaccttgctaaactccaccgtgaggtgagaagtacacctctgacagccgcacctggaggccgaggagacctgaagtatggcacgtatgccttggagaacgagcatttgaatcgactacagtctcaaagagcattactcctacaaggcactgaaagcctgaaccgggctacccaaagcattgagcgttctcatcggattgccacagaaactgatcaaattggtacagaaatcatagaagagttgkgggagcaacgagaccagttggaacgtactaagagcagactggtaaatacaaatgaaaatttgagcaaaagccggaagattcttcgctcaatgtccagaaaagtgataaccaacaagttgctgctctccgtcatcatcttgctggagctagccatcctggtcggtctggtgtattacaaattctttcgacaccattgaacttctgtagggaaagccttgtggaccagcatcttactctgaatgaatggttttacatagggatatgtgttgctgctgtgggctaacagttcaagaatgtattgtcttgtcagactatgggagggagggcaatggaaaacctcgaaaatgtgaaggaacagcaacaagaccagtatgatatactaaggtaataaatgttgtttatgactccttcaaacttaaaaaaaaaaaaaaaaaaaaaaaaa

SEQ ID NO: 307 - DNA, 1224 bp, Mus musculus
cttttttttttttttttttttccgttataatagccatctttatttgtaaaaatccagatataaaacgtaatctttcagtctttccaggttttccttttttacaaaaacaaaaaggcacgtataaaccttgcccgctgtcgtccccgtacacggtgtttctcaggcagccctcccccccgccccgccccccgttacagctacatgcttcattccaggacgtctgcatccccacatgctttggtgctttcctaccagggtagagttccgagctccaagacttgaagtacacaaagagggggtaggggtgggtgcagtgtgtggcacaatgttccacggcgtgcagggcagtgggctagtagtaggtctccttctccacccagccgccagggcgccgcctgataatgagcttccgcacctcgtcatacacaaagatgagaagggagtaggggaaggcacagaaccaccatgtaggtttgaggggatacatcctaarggctgcccccatcccggggcagtaggataagaaagcagcaagggctgtctcttcaaagaggccaaatatcaagatcttgktcttcattccctgctggaagacagaattccttctggtcttgcagatgaccaagtcggcccactgcactaccacaatactgacaaagaacgctgtatggcaggtgaactccacgatcttcctctgctcgtaggtccactgctgcccgtagctgtcctccacatcgttgacccagcggtcatcccaggtctctcggatgcccaacaggtgaaagggcaggaaaccgttctcagccagaatcacaaagtaagtgaagaagcctcccagggcctggatcataccgatctgtccataggccatgctgatcagacgctcgttcacaagtttgtccgttttggggtttctgggctgcctcttcatgatgtcgctctcagcttgctcgtaggccagggagatggcaggaaccatgtcagtgcccaagtcaatgcagaggatggtcacagtccccaggggcagtggaatgtttgcaataataaatatcaagaagggggtgatttccggaatgttactggttagggtgtaagcgatggatttcttcaagttatcaaaaatcagacgaccttcctctactccagtcacaatggaggcaaagttgtcatccagaaggatcatgtcagcagcttgcttggacacatcagagccaacaatcccca

SEQ ID NO: 308 - DNA, 370 bp, Mus musculus
aggtctgcggccctcgcagaacttccagcagcgacatgttgggccagagtatccggaggttcacgacctccgtggtccgtcgcagccactatgaggagggtccggggaagaatttgccattttcagtggaaaacaagtggcggttgctggctatgatgaccgtgtactttggatctgggtttgccgcacctttctttatagtaagacaccagctacttaaaaaataaggatatttaattcatccctttaacagaatgaagaaagtttaagaggtgatctgaaaattggattaaactcttgaactcttatactagaaaaaattgtaaataaactaatgacataaagattcaaaaaaaaaaaaaaaaaaaaa

SEQ ID NO: 309 - DNA, 711 bp, Mus musculus
  misc_feature (697)..(697): n = any nucleotide
agatgaattcaaagagtgcccagggtctggctggtcttcgaaaccttgggaacacgtgcttcatgaactcaattcttcagtgcctgagcaacacccgagagctgagagattactgcctccagaggctgtacatgcgggacctcggccacaccagcagcgctcacacggccctcatggaagagtttgcaaaactaatccagaccatatggacgtcgtcccccaatgatgtggtgagcccatctgagttcaagacccagatccagagatatgcgccacgcttcatgggctataatcagcaggatgctcaggaattccttcgtttccttctggatggtctccacaatgaggtgaaccgggtggcagcaaggcctaaggccagccctgagacccttgatcatctccctgatgaagaaaaggggcgacagatgtggaggaagtatctggaaagggaagacagtcggattggggatctcttcgttgggcagctgaagagctccctcacatgcaccgattgtggctactgctctacagtcttcgatcccttctgggatctctcgttgcccatcgcaaagagaggttaccctgaggtgacgttaatggattgtatgaggctcttcaccaaasaggacatattggatgggtgatgagaagccaacttgctgccgctgccgagccagaaaacgatgcataaaaaagntctctgtccagagg

SEQ ID NO: 310 - DNA, 1211 bp, Mus musculus
  misc_feature (616)..(1040): n = any nucleotide
agggcgcctgagtgaaaagtgtggcaccatggcctctgtgctgtcctacgaaagtctggtacacgccgtggccggagccgtgggaagtgtgactgccatgacagtgttcttccccttggatactgctagacttcggcttcaggtcgatgagaaaagaaagtcaaaaacgacgcatgcagtgctcctggagataattaaggaagaaggcctcctggcaccataccgaggatggtttccagytatttccagtctctgctgctccaattttgtctatttttacacttttaatagcctcaaagcagtgtgggtcaaaggtcagcgttcttctacaggaaaagatctcgtggttgggtttgtagcaggagtggtgaatgtgctgctgacgactccgctctgggtggtaaacaccagactgaagctgcagggggcaaaatttcggaatgaagmcattataccarctaactacaaaggcattatcgatgcattccaccagattattcgagatgaagggatcttggctctgtggaatggcaccttcccctccttgctgwtggwcttcaaccctgccatccaattcatgttctatgaaggcttaaaacggcagcttctaaagaancggatgaagctctcttctctgnatgtgttcatcattggcgcaatagccanagcgattgccnccncagtcacctatcccatgcagacggtacagtcaattctgaggtttggacgtcatagactgaacccagaaaacaggaccctgggaagtcttcggaatgttctctctcttcttcaccagcgagtcaagcgctttggaataatgggactctacaaaggcctggaagctaagctgctgcagacagtgctcacagccgccctcatgttccttgtgtacgagaaactgacagctgctaccttcnccgtaatgggcctgaagagcacacataagcnctgaggcccgcccncaggacagtgtggagacgcaggtgctgcacagggagaagaggaacagttctcctttcccgttggtcccatccncnccncagacattccagtcattggctgtaaaggcatcaaaggctaagcagggagtatagccagctaagcctgtcacctttatggggttttttggtgtttttatggtgtttggttgtatttgggggtagaggttagactgatatgaaaatattgaaaccctgaaaaaaaaaaaaaaa

SEQ ID NO: 311 - DNA, 1078 bp, Mus musculus
  misc_feature (609)..(765): n = any nucleotide
agtagagcccctacggtcatggcggctaccgcccccaaggccgggggttcagctcctgaggcagcgggttctgccgaagctccgttgcagtacagccttcttcttcagtacctggwgggcgacaagcgtcagccccggcttctggagcctggcagcctgggcgggatcccgagtcctgccaagagcgaggagcagaagatgatcgagagggcaatggaaagttgcgccttcaaggctgwgctggcctgtgtaggagggtttgtcttgggaggcgcgtttggtatctttactgctggcattgataccaacgtaggctttgacccaaaggacccttaccggacaccaactgcaaaagaagtcctgaaagacatgggacagagaggaatgtcctatgccaaaaactttgccattgwgggcgccatgttttcatgtactragtgtctggtagagtcttaccggggaaagtcggrctggaagaacagcgtcatcagtggctgcatcactggcggagccatcggcttccgagctggagtaaaggccggggccataggttgtggagggtttgctgctttctctgctgcaatcgattattacctacggtgaaggaancagccgggaagganagaggacgccagccnccttcanagctgctctgtggancagtttctacacacttggactgcttcaggggtgaagccaagtgcatccctctacagttnatggtgtnaaggacncncctcgctgctcgatcgctctctgcagccnttggaacaaccatacttttccmcttgaattccaaccaggtttttcagggraggtggtatggcagattgtctccttgcagtaamcagatcaggagscagtgtgtggmctgttacgcwttaagctttgaaccaggctcagttctgmcctggctcagctccccagctttgaatagggaagtgscagtgtttgcccatasccgggaagctcttcttccttctgtgtgcaagtatccatcatgagtcccagggcaggscaaagtaaacttatgccatcgaggcaaaaaaaaaaaaaaaaaaaaaaaaaaaaaaaaaaa

SEQ ID NO: 312 - DNA, 658 bp, Mus musculus
  misc_feature (610)..(610): n = any nucleotide
aaggccgggaggatgcggccggagcccggaggctgctgctgccgccgcccgatgcgggcgaacggctgcgtcaagaacggggaagtgaggaacgggtacttgaggagcagcaccgccaccgtcgcggctgccggccagattcatcatgtaacagaaaatggaggactgtacaaaagaccgtttaatgaagcttttgaagaaacacccatgctggytgctgtgctcacatatgtgggctatggcgtactcaccctctttggatatcttcgagatttcttgaggcattggagaattgaaaagtgccaccatgcaacagaaagagaagaacaaaaggactttgygtccttgtatcaggattttgaaaacttctatacaaggaacctctacatgagaatcagagacaactggaatcgacctatctgtagtgtgcctggagccaaggtggatatcatggagagaaaatctcatgactataactggtcattcaagtacacagggaatataattaaaggtgtaataaacatgggttcctacaactatcttggatttgcgaggaacactggatcatgtcaggaagcagctgctgaagtcctcaaggagtatggagcangggtgtgcagcactcgtcaggaaattggaaacctggacaagcatgaag

SEQ ID NO: 313 - DNA, 1350 bp, Mus musculus
  misc_feature (69)..(1055): n = any nucleotide
agcggatcttcgggccgggaggacattcggcctctgtgagccgcaaccttgcccagcgagcggttgggngttcgccatcttaggaggatgttctcgtccgtagcgcacctggcgcgggcgaaccccttcaacgcgccacacctgcagctggtgcacgatggcctgtcgggtccccgcagtcccccagctccgccccggcgttcccgccacctggccgccgccgccgnggaagagtacagttgngaatttggctccatgaagtattatgcactgagnggctttggnggggtcttaagttgagggctgacacacactgctgttgttcccctggacttagtaaagygccgcatgcaggtggaccctcagaagtacaaaggcatatttaatggattctccattacactgaaagaagatggcgttcgnggktyggctaaaggatgggccccaactttgattggctattccatgcaagggctctgcaaattcggcttttatgaagtcttcaaagccttatatagcaacatacttggtgaggaaaacacctacctgtggcgcacatcactgtatttagcttcttctgccagagctgaattcttcgctgacattgccctggactcctatggaagctgctaaagttcgaattcaaaacccagcctggktatgccaacaccttgagggaagctgttcccaaaatgtataaagaggaaggcttaaatgcgttctacaagggcgwtgctcctctgtggatgagacagatcccstacaccatgatgaagttcgcctgctttgaacgtactgttgaagctttgtacaaatttgtggttcccaagccccgaagtgaatgtacaaaggcagagcagctggttgtgacatttgtggcaggttacatagctggagtcttctgtgcgatcgtctcccnccctgctgactctgtggtctctgtgctgaataaagagaaaggcagctccgcgtctcaggtcctgcagaggctgggcttcagaggtgtgtggaaggggctcttcgcccgaatcatcatgattggcactctgactgcnctacagtggttcatctacgactctgtgaaggtctacttcaggctccctcgccctcctccccctgagatgccagagtctctgaagaagaagcttgggttaactgagtagatacatcaaagtaacatggactgaatctgcttgttgatcagtgttgaaagtgcaaaggaacttttatatatttgacagtgtaggaaatgtctgttcctgatataattactgtagtacccttgcttaaagcaagagtttcaaacttaatgctaaataaacccatctattcctgaaaaaaaaaaaaaaa

SEQ ID NO: 314 - DNA, 846 bp, Mus musculus
  misc_feature (744)..(744): n = any nucleotide
aggcactgctggcgacatggccgacacggacccgggctatccccgctcgtccatcgaggatgacttcaactacggcagctgcgtggcgtcggccagcgtgcacatccgcatggcctttctcagaaaagtctacagtatcctctctctgcaagtcctcctgactacagtgacctctgccctgttcctgtatttccaagctctgcggacatttgtccatgaaagccctgccttaattgwggwgttwgctctgggatctctgggcttgatctttgcactgactctgcacagacacacgcatcctctgaacctctatctactctttgcatttacactgtcagaatccctggccgwggcagctgttgwtaccttctatgatgtatatctggttctgcaagcgtttataatgactactgcagtctttcttggcttgactgcctatactctacaatcaaagagagatttcaccaaattcggagcagggttgwtwgctggtwtgwggattttgwgcttggcaggattcttgaagctgwttttttacagwgagacgatggagctggtcttggcctctctaggcgccctcctcttctgtgggttcatcatctatgatacacactcgctgatgcacagactctctcccgaagagtacgtgawcgctgccatcagtctctacatggatatcatcaacctcttcctgcacctgktgaagtttctggaagcagttaaataaaaagtaaccgagcagtngttcagagacagktctattatgaaaggacgctttggaattaaactttaaatggttaataattaaaagccawatgtgawctttaaaaaaaaaaaaaaaaaaaa

SEQ ID NO: 315 - DNA, 1058 bp, Mus musculus
agggccgcagcaggcgcgagcgcccaggacctgccagctgagccttccgccgycgccatgggacagaacgacctgatgggcacggccgaggacttcgccgaccagttccttcgagtcaccaagcagtacctgcctcatgtggcgcgcctctgcctgatcagcaccttcctggaggatggcatccgcatgtggttccagwggagtgagcagygtgactatatcgacaccacctggagctgtggctacctgttggcctcatccttcgtgttcctcaacctgctgggacaattgactggctgtgttttggtgctgagcaggaacttcgtgcagtatgcctgctttgggctgtttggaatcatcgcactgcagacaattgcctatagcattttgtgggacttgaagtttctcatgaggaacctggctttaggaggaggtttgctgctgctcttggcagaatcccgctcagaagggaagagcatgtttgctggtgtcccaaccatgcgtgagagctcccccaagcagtacatgcagcttggaggaagggtcctgctggtcctgatgttcatgaccctccttcactttgatgccagcttcttttctatcatccagaacattgtgggcacagctctgatgatcttagtagccatcggttttaaaaccaagctggcagctttgactctcgtcgtctggctgtttgccatcaacgtgtatttcaacgccttctggacaatcccggtatataaacccatgcatgacttcctgaaatacgacttcttccagaccatgtcagtgattggaggcctgctcctgrtggtggctcttggmccagggggtgtgtccatggatgagaagaaraaagagtggtaacacacwgatccctcccctccctggctgaggcrcagggccctggcctggttcagggcaragtcaacaaactgccggcgtttgtgtgtccttctcccttcccctcccttggtaaaggcacagatgttttgagaactttatttgcagacacctgaaaattggagataaaatctttggaaaaaaaaaaaaaaaaa

SEQ ID NO: 316 - DNA, 781 bp, Mus musculus
agcttcttcagtcgggacggggtcagcgagcggctgcttcgtggagcagagaggtgcatcaccaggttcccgatgaacccagagaaccctccaccgtatccgggccccgggccaacagccccatacccaccttatccacaacagccaawggggccaatggggcctatgggagccccacctcctcaggggtacccctacccaccacctcaggggtacccctatcaaggatacccacagtacggctggcagggtggacctcaggagcctcctaagaccacagtgtatgtggtggaagaccaaagaagagacgacctgggcccatccacctgcctcacagcctgctggactgctctgtgttgctgctgcctctgggacatgctcacctgatcacctgatgagcccagctcttccgcttggccgctctgtgccacctccgataagtgtgcctggccccatctcttctgattgctataaagtggctagctctgcgcagacacctctactttctgtcctatggagttttagattagtgagggttactgctatttagttcagtgacttgatctttttaatgttcaaaactcatttcttactgatctttaaaaaatgtgctaaataatttacttttttggccaaaggcttagttatgaaatatatatttataatgtttaaattatacactcaaggtaatggcaatatgtgacacactactagatctctctgctcataccctttatgtttcaataaatctgtccaaaatcaaaaaaaaaaaaaaaaaaaa

SEQ ID NO: 317 - DNA, 766 bp, Mus musculus
gtttccaacaaggacgacatgaagacctcactcaagaaagttgtgaaggagacatcgtatgagatgatgatgcagtgtgtatcgcgaatgctggcccatcccttacacgtgatctcgatgcgatgcatggtgcagtttgtgggacgggaggccaagtacagwggtgtgctgagttctattgggaagatcttcaaggaagaggggctgctgggattcttcgttggcttaatccctcacctcctgggcgatgwggttttcttgtggggctgtaacctgctggcccacttcatcaatgcctacttggtggacgacagcgtgagtgacaccccaggggggctgggaaacgaccagaatccaggttcccagtttagccaggccctggccatccggagctacaccaagtttgygatggggattgcagygagcatgctgacctacccccttyctgctcgctggagatctcatggcagtgaacaactgtgggctgcgggctggactccctccgtattcccctgtgttcaagtcctggatccactgctggaagtacctgagtgtgcagggccagctcttccgcggctccagcctgcttttccgccgggtgtcatcggggtcatgctttgccctggagtaacctaagctgcccgaccaaacatttatggggtcttagcctacccctggtgaggacccatcatctcagatgccccaagggtgactccagcccagccctggcttcatgtccatatttgccatgtgtctgtccagatgt

SEQ ID NO: 318 - DNA, 467 bp, Mus musculus
ctttttttttttttttttttcaccaccaatacatttattcgaggagatgggtctatcttaccacgaggggaggactagatgtcggyctatgtaacctgtgcgtattcgcacccagcacagtgactgaaccctcacacctggcgtcaccagcacagacaagcagatgaggggatggtctgaggagaacatgatttcctattcaggagaaggcaccacccttgtataagaaaattagtgttgggaacatagcgccagcctcccatggcccaggtgtgatggcgcccaatttacaaagcaggaagtggggggcggsggtgcttctggctgactggcaggatgagctgggctagaggtgcagggaagccttgccactgagtgacgtttgcctctgcagcctgcctctgcctgagtacaagatggactccagtacctctaggcaggaaggggatgccaccccaccactgctc

SEQ ID NO: 319 - DNA, 815 bp, Mus musculus
  misc_feature (755)..(755): n = any nucleotide
agggcggaggaagcggactgttccggagctctgcctagccgggcccaacctttgctccagagatcatggctgccgaggatgtggtggcgactggcgccgaccctgagcgagctagagggcggcgggctgctgcacgagattttcacgtcctcctctcaaccctgctcctcctgggcctctgcatcttcctgctctacaagatcgttccgcggggaccagcccggtgcyagtggcgacaacgacgaygacgaaccacccccgctgccccgcctcaagcggcgcgacttcacccctgccgagctgaggcgtttcgatggcgtccaggacccgcgcattctcatggccatcaacggyaaggtgttcgacgtgaccaaaggccgcaagttctacgggcctgaggggccatatggggtctttgccggaagagatgcatccaggggccttgccacattttgcctggacaaagaagcactgaaggatgagtatgacgacctttctgacctcacccctgcacagcaggagaccctgagtgactgggactctcagttcactttcaagtatcatcacgtgggaaaactgctgaaggaaggggaggagcctactgtgtactcagatgatgaagaaccaaaagaygagacagctcggaagaatgaatgaagcattcggtggagcatatctatttttgtattttgcaaaatcatttgtaacattccagtctgtctttacaacatggtgatttcaatatttagaaaagntttgaacttgctgtaaattttttaagtaacatcactagtgacaccgataaaatcaactcg

SEQ ID NO: 320 - DNA, 1252 bp, Mus musculus
agctcgaaagcgacatggcggttctcttaaagctgggcgttctctgcagtggccaaggagctcgagctctcctactccgaagccgggtggtcagacccgcttatgtgtcagcatttctccaggaccagcctacccaaggacggtgtggtacccagcacattcacctgtcaccaagccaccactctggttccaaggctgcatctctccactggaccagtgagagggttgtcagtgttctgctcttggggctgatccctgctgggtacttgaatccctgctctgtggtggactactctctggctgcagccctcaccctgcacagtcactggggccttggacaagtggttaccgactacgttcatggggacaccctgccgaaggctgccagggcaggcctcttggcactctcagctttgacctttgctgggctttgctacttcaattaccacgatgtcggcatctgcagagcggttgccatgctgtggaagctctgacctgggtgcagcactttgattgtgtgcctccttgcctctgctttaccaatgccgttcacctcgcagtgaggggggatgaaggataagcccattggtgggcagaatgtcttctaattacatggttattttcagaatttatttgttgaggaagaggtttgaggagttaggttcgaccattcgtgagtctgtgttccatactccactgagtgtgggcactagctcacagcctcgcggtgagactgaacatttcatgagctcatgktgcctttgaccaccattttcttaaggagagccagctgattgctgtcaggataagagcatctcttcagccaggagggaggcctgttccctcctgagttagactttgcatgaagctcgaaagtattccctttggaacctcccattcttgttcaggtgacaccagctctgttgatggctctgcttctagggaacatttaatcaggagatgctctcaatgactaatttgtctaagtcttaggaaggaggttgaggaaagctggatttagacaagttcaatttagggagttctccttgtttgtggattaaaatatgacagattgcaaacagactactcttcaaatgtatctcaattgtgcagaagtgagctgtccaaaagtataagactaagtgataaactgtcttcccaccgtgggagttgttaatgagaaagaaagtgtactctgaaaaaacaaaatttttaaataaaatattatataatgaaaaaaaaaaaaaaaaaa

SEQ ID NO: 321 - DNA, 1128 bp, Mus musculus
agctaggatctttagtcttcaactcctactgctccttctaacccagcagccccggataatgcagcccaggaggagctcatgatcaccctgatcacaggattggcgtccctcacgtcgagaacctccatgggcatcatcgktgktgggggccggtratttggaaaacaggtgggctggaaactaatctctgtcaccttaagtatgtacggagctctgtacctttatgagaggctgacgtggacgacccgtgcgaaagagagagcgtttaagcagcagtttgtaaactatgcaactgagaagctgcagatgattgtgagcttcaccagtgcaaactgcagccaccaagtacagcaagaaatggccactacttttgctcgactgtgccaacaagtctgatgttactcagaaacatctggaagaggaaattgcaagattatccaaagagatagaccaactggagaaaatacagaacaactcaaagctcttaagaaataaagctattcaactctgaaagtgagctggagaatttttsgaagcagtttctacacccgagcagtggagaatcctaacggcagaggcactgtaggaggaagcggacttggaagatgggaaatgttactttatgaaatgacctcagtacaaattactaactcttagtatcgatgccttgcggagattgtggtaatgacctgtctcaggggttgcacctttggaagtgttgtgattcgccttgtcttagcattagtttggagtaaagactgaattgttaaggttaaatgatgaattcctttagaaacagtggaaccggctgtkcggcccctgagggtgggtcctgcagctcctcaccaggctggctgtctgcggctctcagaagctgcttcctggcatccaggagttagagaccttttcatcctttctcagtgctagttcttgatgcttctttaatgggaatagtgaacttgtttataasccgatttgctcaaacgaggggtgtgggctgctcctgggggggtcctgcaatcactctgtcctcacagcaaggatgtaaccmctactaaacagtttttactttcttttattcccattaaagctgatgtgaaatagtaaaaaaaaaaaaaaaaaaaaaaa

SEQ ID NO: 322 - DNA, 788 bp, Mus musculus
  misc_feature (382)..(774): n = any nucleotide
agtctgcgcccctgtaccctaggtctagtagtgaacccgcacccgrkcaaagaagtctaggccggrgtccgcagmatcagccatcacttcgcacaaagcagcgcagctccgggaccgccgaggaccacagcggcgcggcagcggygcggmgacactcagtgcaccgtatgcccctgcgmccctgccgaggcaaccggagcgccccgagagaccgcgccgccgcggggwccaggtgcagytagcgagcctagcccgcagcgcgcagtcgcgggagagcggggagcggcaagcaacagggagcgggacggcggcgaggcgctcgcgggcccctcctgctgcccgcgcccggcgagctcatggcggccatccgcaagaagctggnggnggngggcgacggcgcgtgcggcaagacgtgcctgctgatcgtgttcagtaaagacgaattccccgaggtgtacgtgcccaccgtgttcgagaactatgtggcggacatcgaggtggacggcaagcaggtggagctggcgctgngggacacggcaggccaggaggactacgatcgtttacggccgctctcctatccggacaccgacgtcatccttatgtgcttctcggtggacagcccggactctctcgagaacatccccgagaagtgggtgcccgaggtaaagcacttctgccccaatgtgcccatcatcctggtggccaacaaaaaagacctgntcagcgacnagcatgtccgacggagctggcccgcatgaagcaggagccagtncngcacggattgacgg

SEQ ID NO: 323 - DNA, 1376 bp, Mus musculus
  misc_feature (646)..(1184): n = any nucleotide
aggtttcctcgccgccggccaagatgaaccgattcttcggaaaagcgaaacccaaggctccgccacctagcttgacggactgcattgggacggtggatagcagggcagaatccattgacaaaaagatttcccggctggatgctgaactagtgaaatataaggatcaaatcaagaagatgagagagggtcctgctaagaacatggtcaaacagaaagccctgagagttttaaagcaaaagcggatgtatgagcaacagcgagacaacctggcccaacagtcctttaacatggagcaagctaattacaccatccagtcactaaaggacaccaagaccacggttgatgccatgaagttgggagtaaaggaaatgaagaaggcatataaggaagtaaaaattgaccagattgaggacttacaagaccagctggaggatatgatggaagatgcaaatgagatccaggaagccctgggccgcagctacggcaccccagagttagatgaggacgacctggaagcagagttagatgcgctgggcgatgagcttctggctgatgaagatagctcctacttggatgaggcagcttccgctcctgcaattccggaaggtgttcccactgacacaaaaaacaaggatggccgtgctgnnggatgaatttgnactgccgcagattcccgcttcgtagacttacaacattcagcacgtgatgtgaaacaacaagagaagtattctgggactaggaaatagttccccgatctgccaaccagatttaggtttctttcctttctttgawgaaaaagctntntacactgctccngnatttttattttttccgntaaggaattcattgcttttgggaaactgtctttactaaaacttgattcntttttttttttttctcttaggaaagactaattgaaaagtacccttgactttgtatgacttgttttcattcattaacaataatctgaaattaaaccaaggagatgagactctgaattctatggtagngtaagtacagtctcagtgngctgatacattgataagataaaagtgattgatgagattgggactgctgatagtatgcttcagaacccttgtctgttgtggtattgtanatgggtttaagtcatggcctcttttgatanattttgttgtgtcatgtgagcaagtcattncacgatntactgttggaatgaactgtctcttcggtatcatgagttactattttgattccatggttccctccagtatactagcctgrcttgtaatgaataatgaatatttcttgatatttaatgkataggrcmtttatttatactcaatwaatatttttcmaaaggaaaaaaaaaaaaaaaaaaaaaaaaaaaaaaaaaa

SEQ ID NO: 324 - DNA, 1004 bp, Mus musculus
agggcggcggccttgggccgcggggaatgggagtcccagggccctgactgagcaccgcccccgcctcccggccgcgccatggctcaggagaagatggagctggacctggagttgcccgcgggcgcgagcccggcggagggcggcggccctggcggcgggggcctgcggaggtctaacagcgcccccctgatccacggcctcagcgactcgtcgccggtgttccaggccgaggcgcccagcgccagacggaacagcacgacgttcccgagccgccacggcctgctgctcccggcctcgccggtccgaatgcacagcagccgcctgcaccagatcaaacaggaggagggcatggacctgatcaaccgagagaccgttcacgagcgcgaggtgcagaccgcaatgcagataagccactcctgggaggaaagtttcagcctgagtgacaacgacgtggagaaatccgcctccccgaagcgcatcgatttcattccggtgtctccggcaccgtccccgacccggggaattgggaagcagtgtttctcaccgtccttgcaaagttttgtgagtagcaacggactgcctccaagtcccattcccagcccaaccacccgatttaccacccggagaagccagagtcccatcaactgcattagaccaagtgttcttggaccattgaaaagaaaatgtgaaatggaaactgattatcagccaaagagatttttccagggcatcaccaacatgctgtcttctgacgttgcacagctgtcagaccccggtgtgtgcgtatcttccgataccctggatggaaacagcagcagtgccggatcttcctgtaactcaccagcgaaagtcagcactaccaccgactctcctgtgtcgcccgcccaagcagcctccccctttattccagtagatgaactttcctcaaagtgactcactcatcttgaatccccctcccccaatggagagacaaaaaaacaaaaacaaacaaaaaaaaaaaaaaa

SEQ ID NO: 325 - DNA, 454 bp, Mus musculus
aggcaatggcggacgtgtctgagaggacgctgcaggtgtccgtgctagtggctttcgcctctggagtggtcctgggctggcaagcgaatcggctgcggaggcgttacctagactggaggaagcggaggctgcaggacaagctggcaacgactcagaaaaagctggacctggcctgagcacgcgctgcagcccgagtccgccgggttctcactccctaagcccaacgcagcccggatcgtgggagccgcgcgacccaggagtcgtccttgcacggcttgcaagaacatggcttgcttcagaaagaaaatagttttgtcttctctaacaacttactttcagcttgtcgaagatgaaaataaaaagcactggagagaaataatttcttgcactttatgaatctatttttaaaataaaaaaattaamcatcaaaaaaaaaaaaaaaaaaaaaaaaaaa

SEQ ID NO: 326 - DNA, 532 bp, Mus musculus
agggactttgttcagatctttttctgatcgtgcatcagctgctggttcattatagccttctcttaagcactgaataagacaaaaaatgaatcttggaagaacaaattcagacatcatcagtaagtctttggggacacagggaatatttgaacttgatttaatctgatgtcttctacaaaacccgctctcccgcattacgttgctgtccccgcagtcgcaggcgcccccggcctggctgcggaacatgttgaagtcgtgtccagtgtggtcgccctggtggaagcactccgcgcacagcgacatgcagggggagatgccgcacgtccggcagcggtaggccacgaagttggccgtccagaccaggccgcagagcgccgcggggtcgtaggcccgcacggccgcgcagaactcctcgtagccgccgccgccggccagaaggcacttacaccactccagggcatcctcctccgcctcgcccgggccgccgccgccgccgcccgccgcctcctcgccgcccgcagccccggccagc

SEQ ID NO: 327 - DNA, 691 bp, Mus musculus
  misc_feature (420)..(690): n = any nucleotide
aggcgacgcgcgcatggaggccggctgaggagcgccgccgcctctctcggtaaggactgtgtctgtgtccccaggcatcctacatcaatcaggaagctgctgtccagccatggagggagaggagaagccagctcaagaggctgacgtggaacctgtggtaacagcaggcacctcagaagcagtgccaagggtgcttgctggagaccctcagaacatctctgatgtggatgccttcaacttgctcctggagatgaaactgaaacgacggcgtgaacgccccaaccttccacgtacagtgacccagctagtggccgaggatgggagcagggtgtatgtggtgggcactgctcacttcagtgatgatagcaagagagatgtagtaaagactatccgggaggtgcaaccggatgtggtcgtggnggagctctgtcagtaccggggtgtccatgctcaagatggacgagaggacgctgctgcgagaggccatggaggtcagcctggagaagctgcagcaggctgtcaggcagaatggnttatgtctggactcatgagatgttgctgcntgnaggtgtctgctcacatcactgagcagctgggcatggcccctggtgncgagttcagggaggccttcaaagaggccagcaaggtaccattctgcaaattccacctgggngaccgaccaatcccagnc

SEQ ID NO: 328 - DNA, 688 bp, Mus musculus
  misc_feature (258)..(666): n = any nucleotide
agcccgtccccagcgctccggcgagctggcctccgctgcagttgagccgggctcgcactatgcagcgcctggccatggacctgcgggtgctgtcccgggagctggcgctctacctggagcaccaggttcgcgtcggcttcttcggctcgggcgtgggcttgtcactgatcttgggattcagcgttgcctatgcttgctactacctgagtagcattgccaagaaaccccagttagtgattggaggggagagtttcagcncgcttcctacaggaccactgtcccgtggtgacagaaacctattacccgacggtctggtgctgggagagccgaggacagacattgctgagacccttcatcacttccaaacccccggtgcaatacaggaacgaactcattaaaactgcagacggaggacagatctcactggactggtttgataacaataacagtgcgtactatgnggatgccagcaccagaccntactatcttgctcttgcctggcctnactgnaacaagcaaggaatcctacatccttcatatgatccatctcagtgaagaattnggatacaggtgngnggnttttaataacanaggagtagcangagaaagtctcttgacaccaccggacttactgctgngcgaacactgaagaacttggaggccgnngtccaccacgtgcacagcctgt

SEQ ID NO: 329 - DNA, 698 bp, Mus musculus
agggcgcagccggcctagcgaggtcaacatgccggtcgccagaagctgggtttgtcgcaaaacctatgtgaccccacggagacccttcgagaagtcgcgtctcgaccaggagctaaagttgattggagagtatggactccggaacaaacgtgaggtttggagggtcaagtttaccctggccaagatccgtaaggcggcccgggagctgttgacgctggacgagaaggatccccggcgtctgtttgaaggcaatgctctcctgcggcggcttgttcgcattggggtgctggacgagggcaagatgaagctggattacatcctgggactgaagattgaggatttcttggagaggcggctgcagacccaggtctttaagctgggcctggccaaatctattcaccatgcccgtgtgctcatccgccaacgtcacattagggtccgcaagcaggtggtgaacatcccatcctttattgttcgcctggactcgcagaagcacatcgacttctccctccgttctccttatggtggcggccgtccaggccgagtgaagaggaagaatgccaagaaaggccagggcggggctggagctggtgatgatgaggaagaggattaattcttggctgaactggaggattgtctagttttccagctgaaaaataaaaaagaattgatacttggaaaaaaaaaaaaaaaaaaaa

SEQ ID NO: 330 - DNA, 384 bp, Mus musculus
agcggcggctctgagtcgccggagccgccccgggacatcatggcggaggtctgcgagcccacccgcccctcggaggacgaggacgaggagcgggagccgctgctgcctcgcgttgcctgggcccagccgcggagggtcgcgcccgggagcgccgtgagaatgcaggcggacgagggcgcggatgtcctccgcgagcccgctaccgacgagccgccggcggtgtccggggaagggtcgatctccgcgagcttgtccaccgagctggatcggacccgcactacgagctcagaaacaaatacattcttggaagatccagaatttgctgatattgtattgaaagcagagcaagcaatagaaattggagttttcccagaaagaatttct

SEQ ID NO: 331 - DNA, 919 bp, Mus musculus
  misc_feature (105)..(408): n = any nucleotide
catcatcgagacaagcaccattaagccttaccgtcgagggttttactgcaatgacgagagcatcaagtatcccctgaaagtcagtgagactataaacgatgctgngctctgngcggnggggatcgtcatcgccatcctggcgatcattacaggggaattctaccggatctattacctcaaggagaagtcccgctccaccactcagaacccgtatgtggcagcncncnataagcaagtggnatgcntcctttnngntgtgccaattagcaagtccttcanngncatcgccaaagtgtccatcgggcgcctaaggcctcacttcctnagcgtctgtgaccctgatttcagtcagatcaattgctccgagggctacattcaganctacaggtgcagaggagaagncagcanagtacaggaggccaggaagtccttcttctcgggccacgcctccttctccatgttcactatgctgtatctggtgctctaccttcaggcccgcttcacctggcgcggggcccgactgctccgccccctcctgcagttcactttgctcatgatggccttctacacgggattgtcacgggtatctgactacaagcatcatcctagcgatgtcctggcaggatttgcccaaggagctctggtggcctgctgcatagtgttcttcgtgtccgacctcttcaagactaagacgagcctctcactgcccgcccctgcgatcaggagggagatcctgtctcccgtggacatcatcgacaggaacaatcaccataacatggtgtagatgctgcggcctccggagcgctttctctgaagcgactgcacgttcctgctgctctccgatctcatcagacagtagaatgtagggaaaagcttttgcccgatggattttgaaaacatttaaaaaaaaaaaaaaaaa

SEQ ID NO: 332 - DNA, 763 bp, Mus musculus
  misc_feature (634)..(747): n = any nucleotide
tgcagggaagacagaaaggcctcccaggccacttggtttattagatcctgaagagaggtgtaggcagtgcccctgggccgctgccacctcctgggggaggacctgtgggaggcacagggccgaacctcgttttgatacacacaaccccatttgagggaaaacaggctgcttcgaagcctgagggatggtgaggggtgatgcctgccatacaggaagccaggtcctgggagggcacaaacgatgaatccatcactgccccagctctgccagcatgcccacctggccctggggaagccaggcaagggagggcacaggcgtgtgagggacacagacagttcctggtgacggcagtagctgctgagcaggagggttcagcaaaactgaccattagagcagccaaggctgcatataggaggtgcgctcgggaaccccaggcactttctctggactccacggtcatggcttctgctggtgatctgcactgcctgcctgtcccctctcctgaaggcactaccttccagaacacagcacggtggtccctcttgtgacaaagtgcttgtgtgtgtgctctagtcatttggtaagcagaagctgccacgggccataccctgccacactacccaagttctgggcnggaaactgctccctgcacagagggccagcgggagcaggaaacgaactcaacttcgctgggcttgccaggcanggcacggtagcagccagggaggttgggacagtgacancagncaggcagacttttttg

SEQ ID NO: 333 - DNA, 421 bp, Mus musculus
agcaaccgcaacctgaccttgaccaagaaggaacctgttggggtctgtggtattgtcatcccctggaactatcccttaatgatgctgtcctggaagacagcagcctgcctggctgccgggaacaccgtgktgatcaagcctgcccaggtgaccccactcacagccttgaagtttgcagagctgacactgaaggctggcattcccaagggtgtggtcaacatcctcccaggatctggctcgctggttggccagagactctcagaccaccctgatgtgaggaaaatagggttcacaggctccacggaggtgggaaaacacatcatgaaaagctgtgccctgagtaatgtgaagaaggtctccctggagctgggtggaaagtcaccccttatcatctttgctgactgtgacctcaacaaagctg

SEQ ID NO: 334 - DNA, 702 bp, Mus musculus
tggaaaggcgatgggcatctctcgggacaactggcacaagcgccgcaagaccgggggtaagcgaaaaccctaccacaagaagcgaaagtatgagctgggacggcccgctgccaacacgaagattggccctcgccgcatacacacagtccgagttcgaggaggcaataagaagtaccgtgccctgagattggatgtggggaacttttcctggggctctgagtgttgtactcgcaaaacaaggatcattgatgttgtctacaatgcatccaacaacgagcttgtccgcaccaagaccctggtgaagaactgcatcgtgcttattgacagcacgccgtaccgacagtggtacgagtcccactatgcactgcccctgggccgcaagaagggggccaagctgactcctgaggaggaagagattttwaacaaaaaacgatcaaagaaaattcaaaagaaatwcgatgaaaggaaaaagaatgccaaaatcagcagcctcctggaggagcagttccagcagggcaagcttytagcctgtattgcctcmcgaccaggccagtgtggcagagcagacggctatgtgctcgaaggcaaggagctggagttctatytgcggaagatcaaagcccggaaaggcmaatgagcctcatgtgtagtgtaataaaggtgtytgctgttctatyttaaaaaaaaaaaaaaaaaaaaaaa

SEQ ID NO: 335 - DNA, 376 bp, Mus musculus
ctggaaagcchggatgaggctcttcatcgctcttcctgtcctgattgtggtcgtagccatgaccttggaaggcccagcccccgcccaggcggccccggatttgtccggaacattggagagcataccggataaactgaaggagtttgggaacactttggaagacaaggcccgggcagccattgaacatatcaaacagaaggaaattttgaccaagacccgggcctggttctcagaggcatttggcaaagtgaaggagaagttgaagaccacgttctcctgagcacctggcgggccacctcgaagcatcaaggacatccacgtaatgtgccaggtccctcttcatcacagaccaataaaaaacgtgtagaaggcaaaa

SEQ ID NO: 336 - DNA, 819 bp, Mus musculus
  misc_feature (447)..(740): n = any nucleotide
aggtttgaacagctttctgaatctgcaaaagaagagctgataaacttcaaacggaagagagtggcagcatttcgaaagaacctaatcgaaatgtctgaactggaaataaagcatgccagaaacaacgtctccctgttgcagagctgcatcgacttattcaagaacaactgacctgtctaytttaaaggcccccawtgtgaaagccagcatcacttgcacttaaatcattactgcaaaagaaatagctttgactagtttaaaatcatgtgaataaatattttgatttctaaaaatcttaacacttaaccatgttggtttaaaaatattttcattgcatgttacttggacataactaatccttttcttatgcatttaatacctcaaagtaggtagaatctaacagattctcctatagactgtcaggaattactaagaaatggtatgganatgtgtgccttctggaaatgagaagcaatagcctggcccatgtctgtctgaacatttcctgtgtctgctcctacagacatgtcaggtgtgaattacagcaaacagtgactgcttgtgggcaggtggcagacactcttctttatttaggtanattttgctccctcttgtcaagatcacttcanatgcttgtacggtaacaagcaagaagatctcatatgtctttttacatcctttacagctccatttttgtaactgggatataaatacttgagagaaaggagaatctanagttntgacactccctgatggtggggcatgtgtgtttgtgctttggatgtttttataaagtatataataaagataatccatgtt

SEQ ID NO: 337 - DNA, 541 bp, Mus musculus
ggcgtgctgacgtcaggcgcgtgcccctgtccggcagccgaggaggccccgcgcagtgctgccaacgccccggcggagaagctgaggacaagatctaatttgaaatattaaaagtggatacaaaactgtttccgaaatgcagacaattaagtgtgttgttgttggtgatggtgctgttggtaaaacatgtctcctgatatcctacacaacaaacaaattcccatcggaatatgtaccaactgtttttgacaactatgcagtcacagttatgattggtggagagccatacactcttggactttttgatactgcagggcaagaggattatgacagactacgaccgctaagttatccacagacagatgtttttctagtatgtttctcagtggtctctccatcctcatttgaaaatgtgaaagaaaagtgggtgcctgagataactcaccactgtccaaagactcctttcttgcttgttgggacccaaattgatctcagagatgacccctctactattgagaaacttgccaagacaaacagaagc

SEQ ID NO: 338 - DNA, 573 bp, Mus musculus
agagggaggctctcagcgatacgtgcccggcccagctttttttttcttcaaaatgtctactgtccacgaaatcctgtgcaagctcagcctggagggtgatcattctacacccccaagtgcctacgggtcagtcaaaccctacaccaacttcgatgctgagagggatgctctgaacattgagacagcagtcaagaccaaaggagtggatgaggtcaccattgtcaacatcctgacaaaccgcagcaatgtgcagaggcaggacattgccttcgcctatcagagaaggaccaaaaaggagctcccgtcagcgctgaagtcagccttatctggccacctggagacggtgattttgggcctattgaagacacctgcccagtatgatgcttcggaactaaaagcttccatgaagggcctggggactgacgaggactccctcattgagatcatctgctcacgaaccaaccaggagctgcaagagatcaacagagtgtacaaggaaatgtacaagactgatctggagaaggacatcatctctgacacatctggagacttccgaaagctgatggtcgccct

SEQ ID NO: 339 - DNA, 1193 bp, Mus musculus
  misc_feature (30)..(797): n = any nucleotide
ctggaaaggctcctcgagggtcggtcccgncacgtctnttcccggtctctctggctctggagtgctttccccccgcggagggtgggagcgcggggacagacgggcgagatgagcaccatgtttgcagacacgctgctcatcgnctttatctccgtgtgcaccgcgctgctcgccgagggcataacctgggtcctggtttacaggacagacaaatacaagagattgaaggcagaagtggaaaaacaragtraaaaattanagaagaagaaggaaactataacagagtcagctggtcgacaacagaagaagaagatagagagacaagaagagaaattaaagaacaacaatagagacctgtcaatggtacggatgaaatccatgttcgcgattggcttttgwtttactgccttaatgggaatgtttaattccatatttgatggtagagtggtggcaaagctccccttcactcctctgtcttacatccaaggactatctcatcggaacctgctgggagatgacaccacggactgctccttcatcttcctgtatatcctctgtaccatgtcaattcgacaaaacatccagaagattcttggcctcgccccttcacgagctgccaccaagcaggctggtggatttctttggcccaccacctccgtctgggaagttttcctgaaggaaagcagaattctgaatttcctgtcatactttttagacattcacatcagacttacagagcacctggccacattctaggtttgtgttatttctcagctatggatatgatcaaatgagancactgcttaactaatgggatagtgctatggtcacggatggcttctttagtaataagtgcactcactggttacatttggatggcttatgtaaccacaaacatcatctttcttgtctcacttgagtatttcagtgtatgtgccatatacacagaagcagactgtccatgaacattaggagaaaaatagtttatgtaacaacttcactgtaattagttgttgtgttttttaaaaagccaaatatgtcactttgtgttcgacaatgagccaaatttttagatgtttcagttagagaaatgtttgatttttttgcatattaaaaagagtattttctgtaatctgaacataaggataagcattggcccaaaaagctaaaataaagttttgtcatgttctttt

SEQ ID NO: 340 - DNA, 707 bp, Mus musculus
  misc_feature (2)..(111): n = any nucleotide
ancctcttcttgggnctaacctaaaaaaaacccccccctggtttacctgggctttttcggaaatttnattncggnccccccctnttaagggagnccccnaagctttgggtncccaaagctcgggattccccttatttaccggcccgccccattgtgctggaaaaggtggaasccsccgccacgatgcagattctttgtgaagaccccttmcggggaaaacccwtcacgctcgaggttgaaccctcggacactatagaaaatgtaaaggccaagatccaggataaggaaggaattcctcctgatcagcaraggctgatctttgctggtaagcagctggaagatggccggactttgtctgactacaacattcaaaaggagtccacccttcatctggtgttgagacttcggggtggtgctaagaaaaggaagaagaagtcttacaccactcccaagaagamcaagcataagaggaagaaggttaagttggctgtgctgaaatactataaggtggatgaaaatggcaaaattagccgacttcgtcgagagtgtccttctgatgaatgtggtgctggagttttcatgggaagccactttgacaggcattactgtggcaagtgttgtctgacttactgctttcaacaaaccagaagacaagtagttgtgtatgcgttaataaaaagaaggaactcgtaaaaaaaaaaaaaaaa

SEQ ID NO: 341 - DNA, 600 bp, Mus musculus
  misc_feature (592)..(592): n = any nucleotide
tttttttttttttttttttttttttttgaaaattaaatcgagatatttaatagacggkgcaaaccataatttcatctgaatataaatgtatgcacacgtttccaatgagctatgtatcaattatcacaacagcaatgaggaccttagagctacgttagaaaactggatagctggaattaactctaacaatcttattcgcttgcaagagaacacatccatttcagatccgtgtcacaaactccaaaagaccaggggggacaggagtgacaagggccaggatggtagacatggggccagtacacaggtgacagacagaaacacagctcttgagggaaccaaaactagatgtgttattacagctaagaacaaggaaaagcctatctatggcttttaaaaaacgacttttctgccagtaccgtacacatcttacagagtgagacacgtcagctgcagggctacatttccagttcgcttcaactgcaaacggccgaccctccacagaggacgcagcagtgggcttagcgcagcccagcaactcagtgttggatttgcaaatacagtttattttacaggaaattcaacattataaamnggggaact

SEQ ID NO: 342 - DNA, 573 bp, Mus musculus
agcaaggaccctgaaaataaacagccgctgctttgcgagtcgccttcttggttcttcgtccgagtctcctccgctgtgggcagctcagacgccgaagctctaactgcagcctatgagcagcaacgaatgcttcaagtgtggacgatctggccactgggccagggagtgccctactggtggaggtcggggtcgtggaatgagaagccgcggcagagggttccagtttgtttcctcgtctctccctgacatctgctaccgctgtggtgagtctggtcatcttgccaaggattgtgatctgcaggaggatgcctgctataactgcggtagaggtggccacattgccaaggactgcaaggagcccaagagagagcgagagcaatgctgctacaattgtggcaagccaggccatctggctcgtgactgtgaccacgcggatgagcagaagtgctattcttgtggtgaatttggacatattcaaaaagactgcaccaaggtgaagtgctataggtgtggtgaaactggtcatgtmgccatcaattgcagcaagacaagtgaagtcaactgttaccgctg

SEQ ID NO: 343 - DNA, 669 bp, Mus musculus
  misc_feature (587)..(643): n = any nucleotide
ttttttttttttttttttcagtcctccacattcccctctttaatatggwattttgttctatatacattgatgaaaatttaaaacaaacaaaaggaactttagtcaaaactccccttcctcctccagctttattggaatagtttaaaattacattttctattttctaggacttcagttgctttttccatctgacttttaaaaactgattacagcaaatgaaacacagttgtctaagtgatatagtatacaaaaataacatcttgatttctgtgaaaatgcatttctctgcaattcctgaatagctccaaattatgctaactctgagcatcggatgtttactctgggttttagatttagtctttgaaaaaatgtgttctacacctttgccatcaccatctgtacgtccagcatcgtcactgggatgaagctgcttctgtgttaggctttgagccagattccctggcatagccttcaacacatttgtcttttcagtttactcccaactcaactgtatattctgagggcctgatgcagatgttactcgatgttatttaatagctactgagggtcagacaatccaaggccgncattatgctaanaattaaaggntatttatggaagcttaaatgcttncagaattggntttaccttctacatggggaacacact

SEQ ID NO: 344 - DNA, 396 bp, Mus musculus
  misc_feature (330)..(381): n = any nucleotide
tttttttttttttttttcgcatataaatgattctttatgcccttcccccatgcagdggggggcggagcagggggaagtcccccgcctcatgcagggaacgggaggtcaatgtttagtagtcttattgccagatgccctgtggccatggggtggggagtggggggtgtcwacagccccttgttttctggcattggtctccctttaccaagtcaccatggtggagccaggggactggaggcttaaagctgaggtaaagatggttctctccaggaagaaaagtggttctccccgcttgcctgctggccatcctcaaggtgagtatgaggcggngtctgtgggaantggaacgtcacccaggggtctctaccctgtggncagtangccggacaatgtctc

SEQ ID NO: 345 - DNA, 442 bp, Mus musculus
  misc_feature (288)..(401): n = any nucleotide
agggaccccgcatcttattakcaaccagggagatttctccattttcctcttgtactacagtgcggcctacaaatctgggatttttttttattacttctttttttaaaaaaactacactwgggctccttttttgtgctcgacttttccacctttttccctccttcctgcgctgctgcttttttgatctcttcgactaaaaattttttatccggagtatttaatcgggtctcttctgtcctcctcgccacccccaccccctccctccggtgtgtgtgccgccgccgctgnatgctgctgctgctgctcgccccgtcgttacaccaaccgaaggctctttgtttcctctcttggatctgccgagtttctttgttgaagaagccagcatgggtgcccagntctcnaagaccgcagcgaagggagaagccaccgtcgagaggccccg

SEQ ID NO: 346 - DNA, 642 bp, Mus musculus
  misc_feature (530)..(612): n = any nucleotide
tttttttttttttttaaatctgggtattggtgtttttatttttttactctttccttcttattcaaaacgtgtagtgttgtaaacctgcctcacaaaatacatcgtaataatttttctttaaaaaaaaaaagaaagaaagaaagaaagaaagaaagaaagaaagaaagaaaggacaagaaccctaagcctttacacccaccaccattttagtggcatattatcttggcaatgttatgcactgcttcaatttcccatagtggcccctatcacttcatttgttattccttttgacccacccatctccttcatatatgggtatgtctatagatctgacaaagaaagtttacacttttttaataaagatgcaaagtatgcaaaaaacattaatactgatgccaaaaaaaaaaaaaaaaaagtaaaagtaaagaaagaaggcagaggaagctgtctaacacgtcctcggcctgtcggaatggtggaaacaactatctgagatgggatctgtggggaagggggcttaaaaaaaacanaaaacaaaaaatttgctgnttaanaaaaatgtgaaatttaaaananaatntttaaaatnaatccctggttgtagacangttnccaaaaaccagtacctggcaccactccaac

SEQ ID NO: 347 - DNA, 438 bp, Mus musculus
ctggaaagctcggttcggtgtctggggtatgaaggtcctgcaaacccgaggcttcgtctcggactcgtcggatagcatggatacgggcgctggctccatccgagaagctggtggagccttcggaaaacgagaaaaggctgaagaggatcggtacttccgagagaagactaaagaacagctggctgccctgaggaaacaccatgaagatgagattgaccaccattcgaaggagatagagcgtctgcagaagcaaattgaacgccataagaagaagatccaacaactaaagaataatcattgaatgcgcgcagtcggtccctcacagagtggcccgtatcactccccacgtctgtagacacatggctttgaatgattactatttggtctgtgtgctactaacagataataaacgatcaccaggaaaaaaaaaaaaaaaaa

SEQ ID NO: 348 - DNA, 478 bp, Mus musculus
agggaggtagaggtgmccgatcctgggggtcggtgttaggccgtgggaggtttttcgcgagactgagccacgcgtctatcttctgtcccagggccgccgtcaccatgggccgcgagtttgggaacctggcgcggatacggcacgtgatctcctacagcttgtcgccctttgagcagcgcgccttcccaagctatttcagcaaaggcatccccaacgtgctgcgccgcactcgcgagcgcatcctgcgcgtggcgccgccgtttgtagtggtctacctgatctacacatggggcaaccaggagtttgagcagtcgaaaaggaagaatccagccatgtatgaaaatgacaagtagacggcctgcacctgggtgacagtcccctgcctctgaaagacccttctctgggagaggaatccmcmctgtagtcttgaagmcaataaactacttatggmcttccaaaaaaaaaaaaaaaaaaaaaa

SEQ ID NO: 349 - DNA, 320 bp, Mus musculus
  misc_feature (225)..(320): n = any nucleotide
cgttgtcttgctccggctgcgcgcattgtcctcagggtcctccgacagggctgctgcggggcccgggacccgcgccctagggacgcgcccccgctgccggtcggcctggcgcggggctctgctagtctgttggcgagcccgtgctaccgggctagtctcgccggggtttttcctgcgaagttgaggaaggggagaagtcccacccgtccgcccagcccagccttncccggcgcgcagcctcgacggggccgtggcaggcgcgacgagggccgccgacggagccctgagagaagtacaaagtggttngtaactgggctcgn

SEQ ID NO: 350 - DNA, 638 bp, Mus musculus
  misc_feature (638)..(638): n = any nucleotide
tttttttttttttttcaggtttgaaacttttgcttgccggtgtcaagcagccaatacagagccttaatggcctatttaaacggggaaatatgcatgaaccaaaatcaacgaggaacgtacacgctgtatagagccatttttccaccggggtctcagatgctctgcaacgtacaccctctcagaccctggctgaaggctggggatgctggctacacaagtcagccaggttcctcggacatcccagccaagacaacaaagtcgtctccaatctcaaagcaagaagacaatcacattaagagaatcgggtgcgtttggctttgtgcacagagcgtggccagcgcccggcggcagacggcgcctcacaggatcacgcaggccgagcagcagcctcgtcgccgttgggctgtgccgcgtagttcatctgcctcacgatctctgcgaatagctcatccactgaggctttgtttttggcccgatgtctccatgaaggggcagctccactcctcggccagggccttaccctcgccataggagacctcacgttcaccctccaagtccaccttgttgcctaccaggatcatggggtacgcgctcgtaccgcttcacgcggatgatctgggcccgcatgggcttgatgn

SEQ ID NO: 351 - DNA, 335 bp, Mus musculus
  misc_feature (4)..(304): n = any nucleotide
tggnaaggggggacttttggtatagcattggaaatgtaaatgagctaaatacctaataaaaaatggaaaaaaaaaaaaaaaaaaaaaaaaagaamtagaaaaaatgtttactgaggtctttatgtctgttgttttgttttgttttagaagtttctgaacgttgttttaggactaaactgggaagccatgtgtcttcgttttggattttatagataacctgaagcattattgagattccttcaaagtcagtaacaacttattgggtgtttcaagccagttgtgagtctctgagtatttgcccagnttcacatttattggtaaaggtcccttacaag

SEQ ID NO: 352 - DNA, 636 bp, Mus musculus
  misc_feature (591)..(628): n = any nucleotide
tttttttttttttttgactcttgctacttgtgaggctcagcaaaaagaattctttacaaggtgtaacccagattatgagaactatagatggaatcagtctgtaccactctgtgccattgtcctctgagtaggttctctgtgactgtagattatttttccagtctataaaaggtcatgggtgctattggaactctagtcaggcatcaactttaggccatcatttctccatcaaaggaaagagagcttctacatttcgcagacatgagcatcctagattacccatagtgctcaagtgtcatttgagtaatttattgagatagcaaaggccttttgcatctgatcatgctgggaagaatggagtaagtttggcatccttacacctaccgagttggcgacagacatattcctcaggaggttggaatgaaatttttgaatgttcagaggaaacctatatacatttaaggtgaaatctaaagaataagagagaaaaacaaaacaaaacaacaaaagaaactcttaccatttgagcactaagaatagtcaaagcagaggaaacaggctagtgcaaaggtccatctagtacaagagagnttatggcttgctccaggaacggacagaaggctagcgnggctggcg

SEQ ID NO: 353 - DNA, 824 bp, Mus musculus
agggacagctcccctttctggaaggtgaaatttttgattctgtgaagccgggactttctgcttttgtggatcagcccaaacagggtgctgagactgtccaggagctcttggaggtggccaaagactcgatccccagaagccactgggaaaggaccccggtggttctgaaagcaacggccggactccgtttgctgcctgagcagaaagcccaggctctgctcttggaggtagaggagatcttcaagaattcacctttcctggtcccagatggcagcgttagcatcatggatgggtcctatgaaggcatactagcctgggttaccgtgaactttctaacaggtcagctgcatggtcgtggccaggagactgtggggacccttgacctggggggtgcctccacccaaatcacgtttctaccccagtttgagaaaaccctggaacaaacacctaggggctaccctcacttcctttgagatgtttaacagcacttttaagctctatacacatagttacttgggatttggactgaaagctgcaagactggcaactctgggagccctggaagcaaaagggactgatggacatacgtttcgaagtgcctgtttaccaagatggttggaagcagagtggatctttgggggtgtgaaataccagtatggtggtaaccaagaaggggagatgggctttgaaccctgctatgcggaagtgctgagggtagtacaggggaaacttcaccagccagaagaagtccgaggaagcgccttctacgctttctcttactactacgatcgagccgctgacacacacttgatcgattataaaaa

SEQ ID NO: 354 - DNA, 705 bp, Mus musculus
aaaagggcagccatggcgcccagccggaatggcatgatactgaagccccacttccacaaggattggcagcagcgagtggacacttggttcaaccagccggcgcgcaagatccgcaggcgcaaggcccggcaggcgaaagcgcgtcgcatcgcccctcgccccgcgtccggccccatcaggcccatcgtgaggtgccctacagtgagataccacaccaaggtccgggctggcaggggcttcagcctggaggagctcagggtggctggcatccacaagaaagtggctcgcaccatcggcatctctgtggacccgaggaggcgaaacaagtccacggagtcactgcaggccaacgtgcagcgcctgaaggagtaccgctccaagctcatcctgttccccaggaagccttctgctccgaagaagggagacagttctgctgaagaacttaagctggccacccagctgacaggacctgtgatgcccatccggaatgtgtacaaaaaggaaaaggccagagttatcacagaagaagagaagaactttaaggcttttgccagtctccgaatggcccgtgccaatgcccgactctttggcatccgagcaaaaagggcgaaggaagctgcagagcaagatgttgaaaagaaaaaataattctgtgttggagagctgcaataaattttccataaagcaaaaaaaaaaaaaaaaaaa

SEQ ID NO: 355 - DNA, 900 bp, Mus musculus
agggcgaagagtgagtgccaaggttcatatgggaaggactttggggtgagcatcttctctatttccagctggcttttctgattttcagaaagaagactcatcaaagatgtccagcaagacggcaagcaccaacagcatagcccaagccaggagaactgtgcagcagctgagattggaagcctccatcgaaagaataaaggtctcaaaagcatcagcagacctgatgtcatactgtgaggagcatgcccggagcgaccctctgctgatgggcataccgacctcagaaaacccgttcaaggataagaagacctgcatcatcttatagtggaccaggaagcgccccttgcctcttaacgcaaaccacagcagcaacctgaagggattccttcagcttacctggtaaccacagctagtaactaaaacacccttctctcggaataatagaccctgaagtctctctttttcaagttgtcctttcttcaccctttactgatttaatacagaatagcaatcttattttctatttgataactatggtatcatattgggttactgtataaggaaaatggcaggggagttgtgggaagcttgtctttacaaaatataattgattaagatatgtcaagacctacattgtctaagcaccggcaaattaaaatgtcgagaatcacttcagtcaaaaacctttatattctgttcttaataatgtttgtgccaacctatatcccatgtaagggatctggggaggaggcatgtgtctacaaccatacctttttgcactatgggcactaaccaccctgaaacttcctgcggtagctccctcccttcagagttacatcattatcctgactctgtgtaggtaaattttcgtgaaatttttgtacaaaaaaaaaaaaaaaa